SEQUENCE LISTING (79 sequences)

SEQ ID NO: 1 - DNA, 445 bp, Homo sapiens
  CDS (1)..(444): Fab7816HC. (SEQIDNO 285)
ctcgagtcgggcccaggacaggtgaagccttcacagaccctgtccctcacctgcactgtctctggtggctccatcggcagtggttcttactcctggaactggatccggcagcccgccgggaggggactggagtggattgggcgaatctctgacagtgggaacaccaatttcaacccctccctcaagagtcgagtcaccatgtcagtggacacgtccaagaaccagttcgccctgaaactgacctctgtgaccgccgcagacacggccacatatttctgtgcgagagggagaggtattttgactggtctctttgactattggggccagggatccctggtctccgtctcctcagcctccaccaagggcccatcggtcttccccctggcaccctcctccaagagcacctctgggggcacagcggccctgggctgcctggtcaaggact

SEQ ID NO: 2 - protein, 148 aa, Homo sapiens
LESGPGQVKPSQTLSLTCTVSGGSIGSGSYSWNWIRQPAGRGLEWIGRISDSGNTNFNPSLKSRVTMSVDTSKNQFALKLTSVTAADTATYFCARGRGILTGLFDYWGQGSLVSVSSASTKGPSVFPLAPSSKSTSGGTAALGCLVKD

SEQ ID NO: 3 - DNA, 316 bp, Homo sapiens
  CDS (1)..(315): Fab7816LC (SEQIDNO337)
tctccagactccctggctgtgtctccgggcgggagggccaccatcaagtgcgcgtccagccagagtgttttggacaactccaaccataagaactccttggcgtggtaccagcagaaaccaggactgcctcctaaactgctcatttactgggcatctacccggtattccggggtccctgaccgattcagtggcagtgggtctgggacagatttcactctcaccatcaacaacctgcaggctgccgatgtggcagtttatttctgtcagcaatattatagtactccgtggaccttcggccaggggaccaaggtggagc

SEQ ID NO: 4 - protein, 105 aa, Homo sapiens
SPDSLAVSPGGRATIKCASSQSVLDNSNHKNSLAWYQQKPGLPPKLLIYWASTRYSGVPDRFSGSGSGTDFTLTINNLQAADVAVYFCQQYYSTPWTFGQGTKVE

SEQ ID NO: 5 - DNA, 455 bp, Homo sapiens
  CDS (1)..(453): Fab5LcxHC (SEQIDNO407)
ggcccaggactggtgaagccttcggggaccctgtccctcacctgcgctgtctctggtggctccatcagcagtagtaactggtggatttgggtccgccagcccccagggaagaggctggagtggattggagaaatcgatcatagtgggactaccaactacaacccgtccctcaagagtcgagtcaccatgtcagtggtcaagtccaagaaccagttctccctgaagctgagctctgtgaccgccgcggacacggccgtctattactgtgcgagaggagcaaaggataactggggattcgactactggggccagggaatcttggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 6 - protein, 151 aa, Homo sapiens
GPGLVKPSGTLSLTCAVSGGSISSSNWWIWVRQPPGKRLEWIGEIDHSGTTNYNPSLKSRVTMSVVKSKNQFSLKLSSVTAADTAVYYCARGAKDNWGFDYWGQGILVTVSSASTKGPSVFPLAPSSKSTSGGTAALGCLVKDYFPEPVTV

SEQ ID NO: 7 - DNA, 376 bp, Homo sapiens
  CDS (1)..(375): Fab5LcxLC
gccatggccgagctcacccagtctccatcctccctgtctgcatctgtaggagacagagtcgccatcacttgccgggcaagtcagaccatcctcgactatttaaattggtatcagcagcgacccgggaaagcccctaagctcctcatctattctgcaacccgtttacaaactggggtcccatcaaggttcactggcagtagatctgggacagatttcactctcaccatcagcagcctacaacctgaagaatttgcaacttattactgtcaacacgattacaatgaccctcgaactttcggccaggggaccaaggtggaagtcaaacgaactgtggctgcaccatctgtcttcatcttcccgccatctgatgagcagt

SEQ ID NO: 8 - protein, 125 aa, Homo sapiens
AMAELTQSPSSLSASVGDRVAITCRASQTILDYLNWYQQRPGKAPKLLIYSATRLQTGVPSRFTGSRSGTDFTLTISSLQPEEFATYYCQHDYNDPRTFGQGTKVEVKRTVAAPSVFIFPPSDEQ

SEQ ID NO: 9 - DNA, 476 bp, Homo sapiens
  CDS (1)..(474): Fab1630HC (SEQIDNO427)
ggaggaggcttggtcaagcctggcgggtccctgagactctcctgcacagcctctggattcactttcagtaacggctggatgagctgggtccgccaggctcctgggaaggggctggagtgggtcggccggattagaagcaaccccgacggtggcacaacagactacgctgcacccttcaaaggcagattcaccatctcaagagatgattcaaaaaatacattgtttctgcaagtgaccagcctgaaaaccgaggacacaggcgtctattactgcatcacagatcggggtgactggaagtggggggtccctagggacctcacctactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 10 - protein, 158 aa, Homo sapiens
GGGLVKPGGSLRLSCTASGFTFSNGWMSWVRQAPGKGLEWVGRIRSNPDGGTTDYAAPFKGRFTISRDDSKNTLFLQVTSLKTEDTGVYYCITDRGDWKWGVPRDLTYWGQGTLVTVSSASTKGPSVFPLAPSSKSTSGGTAALGCLVKDYFPEPVTV

SEQ ID NO: 11 - DNA, 374 bp, Homo sapiens
  CDS (1)..(372): Fab1630LC (SEQIDNO437)
gccatggccgagctcacgcagtctccagacaccctgtctttgtctccaggggaaagagccaccctctcctgtagggccagtcagagtgtcgacagcaactacttagcctggttccagcagaagcctggccaggctcccaggctcctcatttatggtgcgtatagcagggccactggcatcccagacaggttcagtggcagtgggtctgggacagacttcactctcaccatcagcagactggagcctgaggattttgtcgtgtattactgtcagcagtatcttagcccgccgatcaccttcggccaagggacacgactggagactaaacgaactgtggctgcaccatctgtcttcatcttcccgccatctgatga

SEQ ID NO: 12 - protein, 124 aa, Homo sapiens
AMAELTQSPDTLSLSPGERATLSCRASQSVDSNYLAWFQQKPGQAPRLLIYGAYSRATGIPDRFSGSGSGTDFTLTISRLEPEDFVVYYCQQYLSPPITFGQGTRLETKRTVAAPSVFIFPPSD

SEQ ID NO: 13 - DNA, 306 bp, Homo sapiens
  CDS (1)..(306): Fab248HC (SEQIDNO43)
ctcgagtctgggggaggcttggtcaagcctggagggtccctgaggctctcctgtgcagcctctggattcaccttcagtgactactacatgagttggatccgccaggctccagggaaggggctggaatttatatcatacattagtagtggtggtgacaccatacaccacgcagactctgtgaagggccgattcaccatctccagggacaacgccaagaagtcactgtatctccaaatgaacagcctgagagtcgaggacacggccgtatattactgtgcgtgccgtggggtctggggccagggaacc

SEQ ID NO: 14 - protein, 102 aa, Homo sapiens
LESGGGLVKPGGSLRLSCAASGFTFSDYYMSWIRQAPGKGLEFISYISSGGDTIHHADSVKGRFTISRDNAKKSLYLQMNSLRVEDTAVYYCACRGVWGQGT

SEQ ID NO: 15 - DNA, 291 bp, Homo sapiens
  CDS (1)..(291): Fab248LC (SEQIDNO53)
tctccagccaccgtgtctgtgtctccaggggaaagagccaccctctcctgcagggccagtcagagtattagtttccacttagcctggtaccagcagaaacctggccaggctcccagtctcctcatctacggaacatccaccagggccactggtatcccagccaggttcagtggcagtgggtctgggacagagttcactctcaccatcagcagcctgcagtctgaagattctgcggtttattactgtcagcagtatcataactggcctcccctcactttcggcggagggacc

SEQ ID NO: 16 - protein, 97 aa, Homo sapiens
SPATVSVSPGERATLSCRASQSISFHLAWYQQKPGQAPSLLIYGTSTRATGIPARFSGSGSGTEFTLTISSLQSEDSAVYYCQQYHNWPPLTFGGGT

SEQ ID NO: 17 - DNA, 440 bp, Homo sapiens
  CDS (1)..(438): PL12HC22    (SEQIDNO415)
ggggctgaggtgaagaagactgggtcctcagtgaaggtgtcctgcatggtctccggaaacagcttcacccagcgtttcctgcactgggtgcgacaggcccccggacaagcgcttgagtggatggggtggatcacacctttcagtggaaatacctactacgcacagaaattccaggacagactcaccattacgggggacaggtctgtgagtacagcctacatggagttgagcagcctgagatctgacgacacagccatctattactgtgtgatttttggtcttgactactggggcaagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 18 - protein, 146 aa, Homo sapiens
GAEVKKTGSSVKVSCMVSGNSFTQRFLHWVRQAPGQALEWMGWITPFSGNTYYAQKFQDRLTITGDRSVSTAYMELSSLRSDDTAIYYCVIFGLDYWGKGTLVTVSSASTKGPSVFPLAPSSKSTSGGTAALGCLVKDYFPEPVTV

SEQ ID NO: 19 - DNA, 455 bp, Homo sapiens
  CDS (1)..(453): new1
gggggaggcttgctcaagccaggagggtccctgagactctcctgtgtagcctctggattcagcataagcgacttctacatgagttggatccgccaggctccagggaaaggactggagtggatctcatacctcagtggtggcagtacttacaggagccacgcagactctgggaagggccgattcaccatctccagagacaacgccaagaattcactgtttttgcaaatgagtagcctgggagtcgaggacacggccgtgtatttttgtgcgaggcatgtgggagtggcgactgcctttgatatctggggccaagggacagtggtcactgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 20 - protein, 151 aa, Homo sapiens
GGGLLKPGGSLRLSCVASGFSISDFYMSWIRQAPGKGLEWISYLSGGSTYRSHADSGKGRFTISRDNAKNSLFLQMSSLGVEDTAVYFCARHVGVATAFDIWGQGTVVTVSSASTKGPSVFPLAPSSKSTSGGTAALGCLVKDYFPEPVTV

SEQ ID NO: 21 - DNA, 336 bp, Homo sapiens
  CDS (1)..(336): new2
gggggaggcttggtcgagcctggagggtccctgagactctcctgtgaagccactggattcaccttcagtgactactacatgagttgggtccgccaggctcctgggaaggggctggaatggattgcatacattagtactggtagtagttacataaattatgcagactctaagaagggccgattcaccatctccagaaacaacgccaagaactcactgtatctgcaactgaacagcctgagagtcgacgacacggccgtgtattactgtgcgagatcgacacagagtttcgggttgttattacccctcgtcctctttgaccactggggccagggaacc

SEQ ID NO: 22 - protein, 112 aa, Homo sapiens
GGGLVEPGGSLRLSCEATGFTFSDYYMSWVRQAPGKGLEWIAYISTGSSYINYADSKKGRFTISRNNAKNSLYLQLNSLRVDDTAVYYCARSTQSFGLLLPLVLFDHWGQGT

SEQ ID NO: 23 - DNA, 369 bp, Homo sapiens
  CDS (1)..(369): new3
gggggaggcttggtacagcctggggggtccctgagactctcctgtgcagcctctggatccaccttaatcaactatgccatgagctgggtccgccaggctccagggaaggggctggagtgggtctcagttattagtggaactggtgttggcacatactacgcagactccgtgaggggccggttcaccatctccagagacgattccaacaacacggtggatctgcaaatgaatagcctgagagccgaggacacggccgtatattactgtgcgaaagatttccaagtcttcggtgactacatttctctaggctattggggccagggaatcctggtcaccgtcgcctcagcctccaccaagggcccatcggtc

SEQ ID NO: 24 - protein, 123 aa, Homo sapiens
GGGLVQPGGSLRLSCAASGSTLINYAMSWVRQAPGKGLEWVSVISGTGVGTYYADSVRGRFTISRDDSNNTVDLQMNSLRAEDTAVYYCAKDFQVFGDYISLGYWGQGILVTVASASTKGPSV

SEQ ID NO: 25 - DNA, 458 bp, Homo sapiens
  CDS (1)..(456): PL12HC11A  (SEQIDNO 397)
gggggaggcttggcacagcctggggggtccctgagactctcctgtgcagcctctggattcacctttagcagccatggcatgagctgggtccgccaggctccagggaaggggctggagtgggtctcagctattagtgggagtggtggtaacacttactacgcagactccgtgaagggccggttcaccatctccagagacatttccaagaacacgctgtatctgcaaatgaacagcctgagagccgaagacacggccctatattactgtgcgagagggggcgcctatggttcggggagttataagtactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 26 - protein, 152 aa, Homo sapiens
GGGLAQPGGSLRLSCAASGFTFSSHGMSWVRQAPGKGLEWVSAISGSGGNTYYADSVKGRFTISRDISKNTLYLQMNSLRAEDTALYYCARGGAYGSGSYKYWGQGTLVTVSSASTKGPSVFPLAPSSKSTSGGTAALGCLVKDYFPEPVTV

SEQ ID NO: 27 - DNA, 473 bp, Homo sapiens
  CDS (1)..(471): PL12HC28  (SEQIDNO401)
gggggaggcgtggtccagcctgggaggtccctgagactctcctgtgcagcctctggattcaccttcagtagctatgacatgcactgggtccgccaggctccaggcaaggggctggagtgggtggcaattatattggatgatggaagtaataaatactatgcagcctccgtgaagggccgattcaccatctccagagacaattccaagaacacgctgtatctgcaaatgaacagcctgagagctgaggacacggctgtgtattactgtgcgaaagtgcgaatagggaaggtcaataaggtcaataagtcctactttgactcctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtc

SEQ ID NO: 28 - protein, 157 aa, Homo sapiens
GGGVVQPGRSLRLSCAASGFTFSSYDMHWVRQAPGKGLEWVAIILDDGSNKYYAASVKGRFTISRDNSKNTLYLQMNSLRAEDTAVYYCAKVRIGKVNKVNKSYFDSWGQGTLVTVSSASTKGPSVFPLAPSSKSTSGGTAALGCLVKDYFPEPVTV

SEQ ID NO: 29 - DNA, 377 bp, Homo sapiens
  CDS (1)..(375): PL12LC18  (SEQIDNO443)
gccatggccgagctcacgcagtctccaggcaccctgtctttgtctccaggggaaagagtcaccctctcctgcagggccagccagagcgttagtagcaactacttaacctggtaccagcagaaacctggccaggctcccaggctcctcatctatggtgcatccagaagggccactggcatcccagacaggttcagtggcagtgggtctgggaccgacttcactctcaccataagcagactggagcctgaagattttgcagtttattactgtcaacattatggtagctcacctccattccctttcggccctgggaccaaagtggatgtcaaacgaactgtggctgcaccatctgtcttcatcttcccgccatctgatga

SEQ ID NO: 30 - protein, 125 aa, Homo sapiens
AMAELTQSPGTLSLSPGERVTLSCRASQSVSSNYLTWYQQKPGQAPRLLIYGASRRATGIPDRFSGSGSGTDFTLTISRLEPEDFAVYYCQHYGSSPPFPFGPGTKVDVKRTVAAPSVFIFPPSD

SEQ ID NO: 31 - DNA, 374 bp, Homo sapiens
  CDS (1)..(372): PL12LC5   (SEQIDNO441)
gccatggccgagctcacgcagtctccaggcaccctgtctttgtctccaggggaaagagccaccctctcctgcagggccagtcagagcattcgcagcaacttcttagcctggtaccagcagaaacctggccaggctcccaggctcctcatctttggtgcatcgaacagggccactggcatcccagacaggttcagtggcagtgggtctgggacagacttcactctcaccatcagtagactggagcctgaagattttgcggtttattactgtcagcagtatagtagctcaccggacacttttggccaggggaccaagctggagatcaaacgaactgtggctgcaccatctgtcttcatcttcccgccatctgatga

SEQ ID NO: 32 - protein, 124 aa, Homo sapiens
AMAELTQSPGTLSLSPGERATLSCRASQSIRSNFLAWYQQKPGQAPRLLIFGASNRATGIPDRFSGSGSGTDFTLTISRLEPEDFAVYYCQQYSSSPDTFGQGTKLEIKRTVAAPSVFIFPPSD

SEQ ID NO: 33 - DNA, 371 bp, Homo sapiens
  CDS (1)..(369): PL12LC26 (SEQIDNO449)
gccatggccgagctcacccagtctccatcctccctgtctgcatctgtaggagacagagtcaccgtcacttgccgggcaagtcagaccattgccaactatttaaattggtatcagcaaaaaccagggaaagcccctaacctcctgatccaagctgcttccactttgcaaggtggggtcccatcaaggttcagtggcagtcgatctgggacagatttcactctcaccatcaccagtctgcagcctgaggattttgcaacttacttctgtcaacagagtttcagcgccccctggacgttcggccaagggaccaaagtggaaatcaaacgaactgtggctgcaccatctgtcttcatcttcccgccatctgatga

SEQ ID NO: 34 - protein, 123 aa, Homo sapiens
AMAELTQSPSSLSASVGDRVTVTCRASQTIANYLNWYQQKPGKAPNLLIQAASTLQGGVPSRFSGSRSGTDFTLTITSLQPEDFATYFCQQSFSAPWTFGQGTKVEIKRTVAAPSVFIFPPSD

SEQ ID NO: 35 - DNA, 30 bp, Homo sapiens
  CDS (1)..(30): Fab7816-HC2-CDR1
ggtggctccatcggcagtggttcttactcc

SEQ ID NO: 36 - protein, 10 aa, Homo sapiens
GGSIGSGSYS

SEQ ID NO: 37 - DNA, 21 bp, Homo sapiens
  CDS (1)..(21): Fab7816-HC2-CDR2
atctctgacagtgggaacacc

SEQ ID NO: 38 - protein, 7 aa, Homo sapiens
ISDSGNT

SEQ ID NO: 39 - DNA, 45 bp, Homo sapiens
  CDS (1)..(45): Fab7816-HC2-CDR3
tgtgcgagagggagaggtattttgactggtctctttgactattgg

SEQ ID NO: 40 - protein, 15 aa, Homo sapiens
CARGRGILTGLFDYW

SEQ ID NO: 41 - DNA, 36 bp, Homo sapiens
  CDS (1)..(36): Fab7816-LC4-CDR1
cagagtgttttggacaactccaaccataagaactcc

SEQ ID NO: 42 - protein, 12 aa, Homo sapiens
QSVLDNSNHKNS

SEQ ID NO: 43 - DNA, 33 bp, Homo sapiens
  CDS (1)..(33): Fab7816-LC4-CDR3
accttctgtcagcaatattatagtactccgtgg

SEQ ID NO: 44 - protein, 11 aa, Homo sapiens
TFCQQYYSTPW

SEQ ID NO: 45 - DNA, 27 bp, Homo sapiens
  CDS (1)..(27): Fab5Lcx-HC2-CDR1
ggtggctccatcagcagtagtaactgg

SEQ ID NO: 46 - protein, 9 aa, Homo sapiens
GGSISSSNW

SEQ ID NO: 47 - DNA, 21 bp, Homo sapiens
  CDS (1)..(21): Fab5Lcx-HC2-CDR2
atcgatcatagtgggactacc

SEQ ID NO: 48 - protein, 7 aa, Homo sapiens
IDHSGTT

SEQ ID NO: 49 - DNA, 42 bp, Homo sapiens
  CDS (1)..(42): Fab5Lcx-HC2-CDR3
tgtgcgagaggagcaaaggataactggggattcgactactgg

SEQ ID NO: 50 - protein, 14 aa, Homo sapiens
CARGAKDNWGFDYW

SEQ ID NO: 51 - DNA, 33 bp, Homo sapiens
  CDS (1)..(33): Fab5Lcx-LC8-CDR3
tgtcaacacgattacaatgaccctcgaactttc

SEQ ID NO: 52 - protein, 11 aa, Homo sapiens
CQHDYNDPRTF

SEQ ID NO: 53 - DNA, 24 bp, Homo sapiens
  CDS (1)..(24): Fab1630-HC10-CDR1
ggattcactttcagtaacggctgg

SEQ ID NO: 54 - protein, 8 aa, Homo sapiens
GFTFSNGW

SEQ ID NO: 55 - DNA, 30 bp, Homo sapiens
  CDS (1)..(30): Fab1630-HC10-CDR2
attagaagcaaccccgacggtggcacaaca

SEQ ID NO: 56 - protein, 10 aa, Homo sapiens
IRSNPDGGTT

SEQ ID NO: 57 - DNA, 57 bp, Homo sapiens
  CDS (1)..(57): Fab1630-HC10-CDR3
tgcatcacagatcggggtgactggaagtggggggtccctagggacctcacctactgg

SEQ ID NO: 58 - protein, 19 aa, Homo sapiens
CITDRGDWKWGVPRDLTYW

SEQ ID NO: 59 - DNA, 21 bp, Homo sapiens
  CDS (1)..(21): Fab1630-LC12-CDR1
aactaccagagtgtcgacagc

SEQ ID NO: 60 - protein, 7 aa, Homo sapiens
NYQSVDS

SEQ ID NO: 61 - DNA, 33 bp, Homo sapiens
  CDS (1)..(33): Fab1630-LC12-CDR3
tgtcagcagtatcttagcccgccgatcaccttc

SEQ ID NO: 62 - protein, 11 aa, Homo sapiens
CQQYLSPPITF

SEQ ID NO: 63 - DNA, 24 bp, Homo sapiens
  CDS (1)..(24): Fab248-HC14-CDR1
ggattcaccttcagtgactactac

SEQ ID NO: 64 - protein, 8 aa, Homo sapiens
GFTFSDYY

SEQ ID NO: 65 - DNA, 24 bp, Homo sapiens
  CDS (1)..(24): Fab248-HC14-CDR2
attagtagtggtggtgacaccata

SEQ ID NO: 66 - protein, 8 aa, Homo sapiens
ISSGGDTI

SEQ ID NO: 67 - DNA, 21 bp, Homo sapiens
  CDS (1)..(21): Fab248-HC14-CDR3
tgtgcgtgccgtggggtctgg

SEQ ID NO: 68 - protein, 7 aa, Homo sapiens
CACRGVW

SEQ ID NO: 69 - DNA, 18 bp, Homo sapiens
  CDS (1)..(18): Fab248-LC16-CDR1
cagagtattagtttccac

SEQ ID NO: 70 - protein, 6 aa, Homo sapiens
QSISFH

SEQ ID NO: 71 - DNA, 47 bp, Artificial Sequence
  primer
gcgctagtatgccatgggcatgaaagttaaagtactgtccctcctgg

SEQ ID NO: 72 - DNA, 45 bp, Artificial Sequence
  primer
gcgcttcctcgatacctcgagctagaactggtaaaccaggcccag

SEQ ID NO: 73 - DNA, 46 bp, Artificial Sequence
  primer
gcgctagtctgggatccgatgaaagttaaagtactgtccctcctgg

SEQ ID NO: 74 - DNA, 20 bp, Artificial Sequence
  primer
taatacgactcactataggg

SEQ ID NO: 75 - DNA, 30 bp, Artificial Sequence; identical to SEQ ID NO: 35
  primer

SEQ ID NO: 76 - DNA, 1098 bp, Klebsiella pneumoniae
  CDS (1)..(1098): OmpK36
atgaaagttaaagtactgtccctcctggtaccggctctgctggtagcaggcgcagcaaatgcggctgaaatttataacaaagacggcaacaaattagacctgtacggtaaaattgacggtctgcactacttctctgacgacaagagcgtcgacggcgaccagacctacatgcgtgtaggcgtgaaaggcgaaacccagatcaacgaccagctgaccggttacggccagtgggaatacaacgttcaggcgaacaacactgaaagctccagcgatcaggcatggactcgtctggcattcgcaggcctgaaatttggcgacgcgggctctttcgactacggtcgtaactacggcgtagtatacgtcgtaacgtcctggaccgacgttctgccggaattcggcggcgacacctacggttctgacaacttcctgcagtcccgtgctaacggcgttgcaacctaccgtaactctgatttcttcggtctggttgacggcctgaactttgctctgcagtatcagggtaaaaacggcagcgtcagcggcgaaggcgcgaccaacaacggtcgtggttggagcaaacagaacggcgacggcttcggcacctctctgacctacgatatttgggatggcatcagcgctggtttcgcgtactctcactccaaacgtaccgacgagcagaatagtgttccggcactgggtcgtggcgacaacgctgaaacctacaccggtggtctgaaatacgacgccaacaacatctacctggcctctcagtacacccagacctacaacgcaactcgcgccggttccctgggctttgcaaacaaagcgcagaacttcgaagtggttgctcagtaccagttcgacttcggtctgcgtccgtctgtggcttacctgcagtctaaaggtaaggatctggagcgcggctacggcgaccaggacatcctgaaatatgttgacgttggcgcgacctactacttcaacaaaaacatgtccacctatgttgactacaaaatcaacctgctggacgacaacagcttcacccgcaacgccggtatctctaccgacgacgtggttgcactgggcctggtttaccagttctaa

SEQ ID NO: 77 - protein, 365 aa, Klebsiella pneumoniae
MKVKVLSLLVPALLVAGAANAAEIYNKDGNKLDLYGKIDGLHYFSDDKSVDGDQTYMRVGVKGETQINDQLTGYGQWEYNVQANNTESSSDQAWTRLAFAGLKFGDAGSFDYGRNYGVVYVVTSWTDVLPEFGGDTYGSDNFLQSRANGVATYRNSDFFGLVDGLNFALQYQGKNGSVSGEGATNNGRGWSKQNGDGFGTSLTYDIWDGISAGFAYSHSKRTDEQNSVPALGRGDNAETYTGGLKYDANNIYLASQYTQTYNATRAGSLGFANKAQNFEVVAQYQFDFGLRPSVAYLQSKGKDLERGYGDQDILKYVDVGATYYFNKNMSTYVDYKINLLDDNSFTRNAGISTDDVVALGLVYQF

SEQ ID NO: 78 - DNA, 36 bp, Homo sapiens
  CDS (1)..(36): Fab248LC-CDR3
tgtcagcagtatcataactggcctcccctcactttc

SEQ ID NO: 79 - protein, 12 aa, Homo sapiens [sequence omitted]

(19) United States
(12) Patent Application Publication
(10) Pub. No.: US 2012/0165211 A1
(43) Pub. Date: Jun. 28, 2012

(54) Human Antibodies Cross-Reacting With A Bacterial And A Self Antigen From Atherosclerotic Plaques

(75) Inventors: Roberto BURIONI, Milano (IT); Filippo CANDUCCI, Milano (IT); Massimo CLEMENTI, Milano (IT); Federico MAISANO, Lodi (IT)

(73) Assignee: BRACCO IMAGING S.P.A., Milano (IT)

(21) Appl. No.: 13/350,527

(22) Filed: Jan. 13, 2012

Related U.S. Application Data
(63) Continuation-in-part of application No. 12/679,109, filed on Aug. 27, 2010.

(30) Foreign Application Priority Data
Sep. 20, 2007 (EP) 07116856.1
Jul. 18, 2008 (EP) 08160692.3
Jan. 14, 2011 (EP) 11150941.0
Jul. 27, 2011 (EP) 11175564.1

(51) Int. Cl. G01N 33/53; G01N 33/554; C40B 30/04; C07K 16/12

(52) U.S. Cl. 506/9; 530/389.5; 435/7.32; 435/7.92

(57) ABSTRACT

The present invention refers to human antibodies derived from human antibody libraries prepared from atherosclerotic plaques. It further refers to human antibodies able to immunologically recognize both human transgelin or fragments thereof and a protein with at least 50% similarity to OmpK36 (Outer membrane protein, Klebsiella, K36; GI: 295881594) or fragments thereof. Human transgelin is preferably transgelin 1 (Accession N° Q01995, GI:48255907). The antibodies further recognize an antigen in the atherosclerotic plaque and are useful for the preparation of immunodiagnostic reagents or assays to detect atherogenic diseases. The invention also relates to the use of anti-TAGLN monoclonal antibodies, showing cross-reactivity with a bacterial antigen having at least 50% similarity with OmpK36, for detecting antigens in the atherosclerotic plaque or as atherosclerotic related reagents in an immuno-competition assay.

### CROSS-REFERENCE TO RELATED APPLICATIONS

This application is a continuation-in-part of U.S. Ser. No. 12/679,109, filed Aug. 27, 2010, which is a national stage of PCT/EP2008/062408, filed Sep. 18, 2008, which claims benefit of EP 08160692.3, filed Jul. 18, 2008 and EP 07116856.1, filed Sep. 20, 2007 and claims benefit to and priority of EP 11150941.0, filed Jan. 14, 2011 and EP 11175564.1, filed Jul. 27, 2011. All of the above applications are hereby incoroporated by reference in their entirety.

### FIELD OF THE INVENTION

The present invention relates to a method for preparing new oligoclonal antibodies, the antibodies themselves as well as fragments thereof and their uses as well as the antigen and ligands thereof. In particular, the present invention encompasses antibodies or fragments thereof that are directed against antigens possibly found in the coronary plaque. The present invention further relates to the nucleotide sequences coding for these antibodies and amino acid sequences of the antibodies or fragments thereof for use in, for example, immunoassays, as well as to the ligands of these antibodies or fragments thereof. Further, the invention encompasses diagnostic and therapeutic applications related to the use of said antibodies or fragments thereof or of their ligands.

### BACKGROUND OF THE INVENTION

### Antibody Structure

There exist five types of antibodies (also called immunoglobulins): IgG, IgA, IgD, IgM and IgE. The structure of IgG, depicted in FIG. 1, comprises two light chains of a molecular weight of approximately 23 KDa and two heavy chains of about 53-70 KDa. The four chains being linked to each other by disulfide bonds in a “Y” configuration.

Heavy chains are classified as γ, η, α, δ and ε with some subclasses among them, while light chains are classified as either κ or λ.

Each heavy chain comprises a constant region and a variable region, the latter being located at the N-terminal end of the immunoglobulin molecule of approximately 100 amino acids in length.

In particular, the most variable part of the immunoglobulin (Ig) heavy and light chains is the third complementarity-determining region (CDR3), a short amino acid sequence which is formed by the junctions between the V-D-J gene segments. CDR3 is found in the variable domains of antigen receptor (e.g. immunoglobulin and T cell receptor) protein that complements an antigen.

The variability of the CDR3 portion is responsible for the elevated number of antibodies produced and which are specific for any antigens; said variability is determined by the rearrangement of the V, D and J minigenes that occurs in the bone marrow during the generation of mature B cells.

After this first rearrangement has occurred, when the mature B cell encounters an antigen, further hypermutational events are responsible for the increased affinity of the antibody for that specific antigen.

“Lineage trees” or “dendrograms” have frequently been drawn to illustrate diversification, via somatic hypermutation of immunoglubulin variable region genes. More in particularly, the generation of lineage trees to visualize the lineage relationships of B cells mutant in the germinal centers has been used in the past to confirm the role of the germinal center as the location of somatic hypermutation and affinity maturation.

### Acute Coronary Syndrome

The acute coronary syndrome (also referred to as ACS) is the manifestation of a plaque rupture in a coronary artery.

The rupture or the erosion of an atherosclerotic plaque, with the subsequent formation of thrombus and occlusion of the artery may cause myocardial infarction and unstable angina (see, for a general reference, “New insights into atherosclerotic plaque rupture” D. M. Braganza and M. R. Bennett, Postgrad. Med. J. 2001; 77; 94-98).

An atherosclerotic event begins as a subendothelial accumulation of lipid laden monocyte derived foam cells and associated T cells which form a non-stenotic fatty streak. With progression, the lesion takes the form of an acellular core of cholesterol esters, bounded by an endothelialised fibrous cap containing smooth muscle cells (VMSC) and inflammatory cells (both macrophages and T lymphocytes). Also present in the advanced lesions are new blood vessels and deposits of calcium hydroxyapatite may also appear in advanced lesions (see as a general reference, “Coronary disease: Atherogenesis: current understanding of the causes of atheroma” Peter L. Weissberg, Heart 2000; 83; 247-252).

The extracellular lipid core of the plaque is composed of free cholesterol, cholesterol crystals and cholesterol esters derived from lipids infiltrated the arterial wall or derived from the dead foam cells. The lipid core may affect the plaque by causing stress to the shoulder regions of the plaque; in addition, the lipid core contains prothrombotic oxidized lipids and it is further impregnated with tissue factors derived from macrophages in which the lipid core materials are highly thrombogenic when exposed to circulating blood (see, for instance, “Mechanism of Plaque Vulnerability and Rupture” Prediman K. Shah, Journal of the American College of Cardiology 2003).

The stability of the plaque depends also upon the vascular smooth muscle cells (SMCs) content of the plaque, as they are capable of synthesising the structurally important collagens types I and III. In contrast, macrophages and other inflammatory cells may release matrix metalloproteinases (MMPs) which degrade collagen and extracellular matrix, thus potentially weakening the plaque (see, “New insights into atherosclerotic plaque rupture” D. M. Braganza and M. R. Bennett, Postgrad. Med. J. 2001; 77;94-98).

The structural components of the fibrous cap include matrix component such as collagen, elastin and proteoglycans, derived from SMCs. Said fibrous cap protects the deeper components of the plaque from contact with circulating blood and has been observed to thin out in the vicinity of the rupture (see, for example, “Mechanism of Plaque Vulnerability and Rupture” Prediman K. Shah, Journal of the American College of Cardiology 2003).

Ruptured plaques have been shown to have several histomorphologic features with respect to intact plaques. Therefore, when they are present, they are thought to indicate vulnerability to plaque rupture (see, for instance, “Mechanism of Plaque Vulnerability and Rupture” Prediman K. Shah, Journal of the American College of Cardiology 2003).

One possible cause of plaque formation is thought to be repeated injury to the endothelium caused by the four “major” risk factors: smoking, hypertension, diabetes and hyperlipidaemia (high level of LDL and low level of HDL). Endothelial dysfunction following injury, moreover, plays a crucial role in plaque initiation, as lipids may pass more easily from the bloodstream into the tunica intima.

The rupture of a vulnerable plaque may occur either spontaneously, i.e. without occurrence of any of the above mentioned triggers or following a particular event, such as an extreme physical activity, a severe emotional trauma and stresses of different nature or acute infection.

Plaque rupture often leads to thrombosis with clinical manifestations of an ACS.

The thrombotic response to a plaque rupture is probably regulated by the thrombogenicity of the constituents exposed on the plaque; generally, the plaque rupture develops in a lesion with a necrotic core and an overlying thin fibrous cap heavily infiltrated by inflammatory cells. A luminal thrombus further develops due to the physical contact between platelets and the necrotic core (see, for example, “Pathologic assessment of the vulnerable human coronary plaque” F. D. Kolodgie et al. Heart 2004; 90; 1385-1391).

Rupture or erosion of the fibrous cap exposes the highly thrombogenic collagenous matrix and lipid core to circulation leading inevitably to platelet accumulation and activation. This in turn leads to fibrin deposition and thrombus formation which may result into vessel occlusion, the latter being not inevitable, such as in the case of silent episodes (see, for instance, “Coronary disease: Atherogenesis: current understanding of the causes of atheroma” Peter L. Weissberg, Heart 2000; 83; 247-252).

Until recently, atherosclerosis was thought of as a degenerative and slowly progressive disease causing symptoms through its mechanical effects on blood flow, while it is now understood to be a dynamic inflammatory process that is eminently modifiable. Recent researches on cellular and molecular events underlying development and progression of atherosclerosis, focus the attention on the dynamic interaction between the plaque components that dictates the outcome of the disease (see, as a general reference “Coronary disease: Atherogenesis: current understanding of the causes of atheroma” Peter L. Weissberg, Heart 2000; 83; 247-252).

There are contrasting data for a relation between coronary syndrome and several pathogens to be assessed.

In a prospective study (see, for example, “Impact of viral and bacterial infectious burden on long term prognosis in patients with coronary artery disease” Rupprecht H. J. et al., Circulation 2001, Jul. 3; 104(1): 25-31) it was described the relation between stroke and 8 different pathogens (Herpes simplex virus 1-2, Epstein-Barr, Cytomegalovirus, Haemophilus influenzae, Mycoplasma pneumoniae, Helicobacter pylori and Chlamydia pneumoniae) in a group of 1018 patients; there was found an increase in mortality, related to the serum positivity for six to eight pathogens of 7% and 12.6% respectively.

De Palma and his group (“Patients with Acute Coronary Syndrome Show Oligoclonal T-Cell Recruitment Within Unstable Plaque” De Palma et al. Circulation 2006, 113: 640-646) conducted a study on the T cells repertoire recovered from blood sample and also directly from the coronary plaque of patients with acute coronary syndrome.

Inflammation is now understood to play a key role in the development of atherosclerotic disease. In particular, as the inflammatory process becomes chronic, remodelling and formation of new intima is triggered. Furthermore, the interaction between lymphocytes and dendritic cells (DCs) within the neointima might be responsible for the development of a local immune response against exogenous and endogenous atherogenic antigens. Thus, atherosclerosis is considered to belong to the group of chronic inflammatory diseases for which whose development and complications are mainly due to the cellular components of the immune system . The chronic accumulation of monocytes/macrophages, smooth muscle cells, and T-lymphocytes in response to the accumulation and release of pro-inflammatory molecules within the arterial wall constitutes the hallmark of a developing atherosclerotic plaque.

The finding rported in the parent application, U.S. Ser. No. 12/679,109, that antibodies are produced within the plaqueseems to confirm the involvement of a local immune response (see also Burioni R. et al. J Immunol., 2009, 183:2537-2544).

Although most of the antigenic stimuli that occur within atherosclerotic plaques come from modified self-molecules, the immune response triggered is remarkably similar to inflammatory reactions mounted against microbial organisms. Among the list of atherosclerosis-related antigens, ranging from oxidised low-density lipoproteins (oxLDL), heat shock proteins (HSP) to protein components of the extracellular matrix such as collagen and fibrinogen, transgelin 1 (also called SM22) has never been mentioned (see for a review: Milioti N. et al. Clin. Dev. Immunol., 2008, 2008:723539) even though transgelin 1 is known to be physiologically expressed in smooth muscle cells within the arterial wall, in particular as one of the earliest markers of smooth muscle cell differentiation due to its role in cytoskeleton organization (Assinder S. et al. Int. J. Biochemistry & Cell Biology, 2009, 41:482-486).

Patents on transgelin uses include the following, for example: EP0914426 describes DNA sequences including a fragment of the sequence ahead of the coding portion of the murine SM22 protein gene. Said sequence and vectors have been proposed for treatment of coronary diseases, in particular restenosis.

WO2007035451 provides methods of modulating angiogenesis in an individual, involving modulation of nicotinic acetylcholine receptor (nAChR), bFGF receptor, and VEGF receptor acting on the expression of, among others, TAGLN the gene encoding transgelin 1.

WO2007140972 proposes Transgelin-3 as a biomarker for Alzheimer's disease. U.S. Pat. No. 5,837,534 and U.S. Pat. No. 6,015,711 provide for sequences encoding the murine SM22 alpha promoter (arterial SMC-specific promoter), and gene transfer vectors containing the same, to target gene expression in arterial smooth muscle cells (gene therapy of the arterial vessel wall).

Thus, although the expression of SM22 has been related to disorders involving smooth muscle cell activation, in particular in vascular remodelling, this protein does not appear to have been proposed as a relevant marker for atherosclerosis or predisposition to the development of this disease.

The present invention is based, in particular, on the new finding that transgelin and a category of bacterial antigens are immunologically related and that the specificity of antibodies for these two antigens might be diagnostically relevant for patients suffering from Acute Coronary Syndrome or during the development of this disease, in the atherogenic process.

As a matter of fact, the role of foreign antigens, such as viruses and bacteria, in atherogenesis as causative or bystander participants in its development, has already been addressed and still is a controversial issue. It does not appear that Klebsiella nor the outer membrane proteins have ever been associated with the development of atherosclerotic diseases, introducing another level of complexity to the analysis. Thus, in the present scenario, the disclosure of reagents with this new cross-reactivity provides extremely valuable technical advancements, either in the diagnostic or research field related to atherosclerosis.

### SUMMARY OF THE INVENTION

The instant application refers to SEQ ID NOS: from the sequence listing submitted in the parent case, U.S. Ser. No. 12/679,109, which is included herein as FIG. 25, as well as SEQ ID NOS: from the sequence listing submitted with the instant application. The SEQ ID NOS: from the parent sequence listing (FIG. 25) are reported herein in italics. A correspondence chart of pertinent SEQ ID NOS: from the parent application sequence listing (FIG. 25) and the instant sequence listing appears herein after the List of Embodiments and before the DETAILED DESCRIPTION OF THE INVENTION.

Additionally, where pertinent, the SEQ ID NOS: from the parent case are identified in italics and parentheses after the SEQ ID NOS: from the instant sequence listing.

The following are illustrative examples of the objects of the invention, that will be more apparent from the teaching of the whole disclosure.

A first object of the invention includes the isolated polynucleotide sequences coding for the heavy chains of the antibodies and corresponding to the odd-numbered Sequence ID from 1 to 51, 65 to 105, 191 to 209, 253 to 295, 345 to 349, 371 to 383 and 395 to 427.

A second object of the invention is thus represented by the amino acidic sequences coding for the heavy chains of the antibodies and corresponding to the even-numbered Sequence ID from 2 to 52, 66 to 106, 192 to 210, 254 to 296, 346 to 350, 372 to 384 and 396 to 428.

A third object of the invention are the isolated polynucleotide molecules coding for the light chains of antibodies and corresponding to the odd-numbered Sequence ID from 53 to 63, 107 to 189, 211 to 251, 297 to 343, 351 to 369, 385 to 389 and from 429 to 453.

A fourth object of the invention is thus represented by the amino acid sequences coding for the light chains of antibodies and corresponding to the even-numbered Sequence ID from 54 to 64, 108 to 190, 212 to 252, 298 to 344, 352 to 370, 386 to 390 and from 430 to 454.

A fifth object of the present invention includes an expression vector, comprising one or more of the isolated polynucleotide molecules, as well as the complementary sequences thereof, encoding for the amino acid sequences corresponding to the even-numbered Sequence ID from 2 to 390 and from 396 to 454 and the homologous sequences thereof.

An additional object of the present invention includes an expression system comprising one or more of the isolated expression vector of the invention and a suitable host cell.

As further object of the present invention, there is provided a host cell comprising one or more of the expression vectors of the present invention.

An additional object of the present invention includes a process for the production of recombinant antibodies or fragments thereof including the use of the expression system of the invention comprising one or more of the isolated polynucleotide molecules comprising the odd-numbered Sequence ID from 1 to 389 and from 395 to 453 as well as the complementary and homologous sequences thereof.

A further object of the invention encompasses the isolated recombinant antibodies or fragments thereof produced by the host cell comprising the expression vector of the present invention.

It is another object of the present invention to provide an immunoassay including the use of one or more of the amino acid sequences corresponding to the even-numbered Sequence ID from 2 to 390 and from 396 to 454 and the homologous sequences thereof.

In an additional embodiment of the invention, there is provided a therapeutic composition comprising the antibodies of the present invention or any fragments thereof and a therapeutic moiety linked thereto.

In a further embodiment of the invention, there is provided a diagnostic composition comprising the antibodies of the invention or fragments thereof linked to a diagnostic moiety.

A still further embodiment of the present invention is a ligand that specifically binds at least one of the antibodies of the invention or to any fragments thereof.

A further object of the invention is a method for the screening of molecules for identifying those having the most binding affinity for the antibodies of the present invention or for any fragments thereof.

As an additional embodiment of the present invention there is an immunoassay, which includes the use of the ligand identified according to the present invention.

A still additional embodiment of the invention, is a therapeutic or diagnostic composition comprising the ligand of the present invention, covalently linked or otherwise functionally associated with a therapeutic or to a diagnostic moiety or entity.

An additional embodiment of the invention is represented by the use of immunosuppressant, immunomodulant or antinfective agents for the preparation of pharmaceutical compositions for the treatment of coronary diseases, such as the acute coronary syndrome or of immuno-related pathologies.

In a further embodiment of the invention, there is provided a method for the identification of the etiologic agent responsible for the development of immuno-related pathologies.

An additional embodiment of this invention is an amino acid consensus sequence of a putative ligand possibly found in the coronary plaque.

In a further embodiment of the invention, there are provided four peptides showing the consensus sequence.

According to further embodiments the invention comprises human recombinant antibodies with heavy and light variable chains derived from the atherosclerotic plaque and selected among those with immunological specificity for both human transgelin (or fragments thereof) and a protein with at least 50% similarity to OmpK36 (Outer membrane protein, Klebsiella, K36; GI: 295881594 or SEQ ID NO:76) or fragments thereof.

By similarity the Applicant means protein-protein primary structure comparison based on both amino acid identity and similarity as defined for example in: A Structural Basis of Sequence Comparisons An evaluation of scoring methodologies Johnson, M. S., Overington, J. P. 1993 Journal of Molecular Biology 233: 716-738 or: Improved tools for biological sequence comparison. Pearson, W. R., Lipman, D. J. 1988 Proceedings of the National Academy of Sciences USA 85:2444-2448; or: Searching Protein Sequence Libraries: Comparison of the Sensitivity and Selectivity of the Smith Waterman and FASTA algorithms. Pearson, W. R. 1991 Genomics 11: 635-650.

Human transgelin is preferably transgelin 1 (Accession N° Q01995, GI:48255907).

Preferred antibodies comprise Heavy variable chains selected from the group consisting of: SEQ ID NO:2 (SEQ ID NO:286), SEQ ID NO:6 (SEQ ID NO:408) and SEQ ID NO:10 (SEQ ID NO:428) and at least one of the light chains selected from the group consisting of SEQ ID NO:4 (SEQ ID NO:338), SEQ ID NO:8 and SEQ ID NO:12 (SEQ ID NO:438).

Even more preferably, the Heavy chain variable region consists of SEQ ID NO:2 (SEQ ID NO:286) or SEQ ID NO:10 (SEQ ID NO:428) and the Light chain variable region consists of: SEQ ID NO:4 (SEQ ID NO:338), or SEQ ID NO:12 (SEQ ID NO:438).

Selected Fab's further comprise the following heavy and light chain pairing wherein the heavy and light chains comprise or consist respectively of: Heavy Chain SEQ ID NO:18 (SEQ ID NO:416) and light chain SEQ ID NO:30 (SEQ ID NO:444) or SEQ ID NO:32 (SEQ ID NO:442); Heavy Chain SEQ ID NO:20 and Light Chain SEQ ID NO:32 (SEQ ID NO:442) or SEQ ID NO:34 (SEQ ID NO:450); Heavy Chain SEQ ID NO:22 and Light Chain SEQ ID NO:32 (SEQ ID NO:442); Heavy Chain selected from: SEQ ID NO:24 and SEQ ID NO:26 (SEQ ID NO:398) and Light Chain SEQ ID NO:30 (SEQ ID NO:444); Heavy Chain SEQ ID NO:28 (SEQ ID NO:402) and Light Chain SEQ ID NO:12 (SEQ ID NO:438), SEQ ID NO :30 (SEQ ID NO:444) or SEQ ID NO :32 (SEQ ID NO:442).

The antibodies further recognize an antigen having immunological similarity to transgelin or fragments thereof and a protein with at least 50% similarity to OmpK36 (GI: 295881594 or SEQ ID NO:76) in the atherosclerotic plaque and are useful for the preparation of immunodiagnostic reagents or assays for atherogenic disorders, able to detect atherogenic ischemic or occlusive evolution in an arterial vessel, Acute Coronary syndrome (and related cardiovascular diseases selected from the group consisting of: unstable angina, ST Elevation Myocardial Infarction (STEMI), nonSTEMI myocardial infarction)), or intra-cerebral occlusive disease or peripheral artery occlusive diseases or a predisposition to any of those diseases. According to a preferred embodiment the method for detecting antibodies against an antigen in an atherosclerotic plaque comprises allowing an unknown biological sample to react with human transgelin or fragments thereof, optionally in competition with any of the antibodies according to the invention. More preferably the immunoassay is a Western-blot or an ELISA.

As a related embodiment the invention further provides a method for screening a polypeptide, such as an antibody or fragments thereof, and peptide libraries where panning is carried out independently and sequentially on each of the antigen: transgelin, preferably transgelin 1 (Accession N° Q01995, GI:48255907) or fragments thereof and a protein with at least 50% similarity to OmpK36 (Outer membrane protein, Klebsiella, K36; GI: 295881594) or fragments thereof.

According to a further embodiment the present invention also relates to transgelin-1 as a marker of the presence of an atherosclerotic plaque.

The invention also relates to the use of anti-TAGLN monoclonal antibodies, showing cross-reactivity with a bacterial antigen having at least 50% homology with OmpK36, for the detection of antigens in the atherosclerotic plaque or as reagents in immuno-diagnostic assays for atherosclerotic related disorders.

### BRIEF DESCRIPTION OF THE FIGURES

FIG. 1 is a schematic representation of the structure of an IgG antibody molecule and of a Fab fragment thereof.

FIG. 2 represents the recombinant pattern for the production of antibodies.

FIG. 3 represents the number of functional gene segments in human immunoglobulin loci.

FIG. 4 is a schematic representation of the preparation of the antibodies or fragments thereof according to the present invention.

FIG. 5 shows the analysis of the VDJ and VJ gene for the heavy chains of the coronary plaque sample.

FIG. 6a graphically shows the homology percentage of light chains of peripheral blood samples compared to coronary plaque samples.

FIG. 6b graphically shows the homology percentage of heavy chains of peripheral blood samples compared to coronary plaque samples.

FIG. 7 shows the nucleotide sequence alignment of two clonal variants of heavy chain from a plaque (#8 e #24).

FIG. 8 shows the amino acid sequence alignment of two clonal variants of light chain from a plaque (#8 e #15).

FIG. 9 shows the alignment of the aminoacidic sequence of β-globin (as internal control) and standard β-globin L48931.

FIG. 10 shows the sequences of the primers used according to the present invention. A: the primers annealing to the 5′ of variable regions of K light chains; B: primers annealing to the 3′ of constant region of K light chains; C: primers annealing to the 5′ of variable regions of heavy chains; D: primers annealing to the 3′ of constant regions.

FIG. 11 is a schematic representation of a lineage tree.

FIG. 12 is a mutational lineage tree of clonally related groups of light chains

FIG. 13 is a mutational lineage tree of clonally related groups of heavy chains.

FIG. 14 shows the ELISA results for Fab 24 on Hep-2 cell lysate.

FIG. 15 shows the ELISA results for Fab 24 on syntetic ligands.

FIG. 16. shows a histological section of an atheromatous area of the carotid plaque (large image in the middle), with indication of the regions of enlargment of three areas (*; # and ?) stained by immunofluorescence with the 7816 FLAG in the relevant panels (left and right small panels: *; # and ?) .

FIG. 17. Western blotting sections showing Fab7816 binding with an antigen in the carotid lysate. Each number corresponds to a distinct patient; each decimal to a distinct section of the same lesion. cubV: umbilical vein.

FIG. 18. Western blot assay on pathogen lysates with Fab7816. Arrows show that Fab7816 recognizes with high specificity and affinity an antigen present in Klebsiella pneumoniae and in Proteus mirabilis.

FIG. 19 . ELISA results for 7816 IgG against the bacterial antigen ompK36.

FIG. 20. 2D Electrophoresis (Panel A) and 2D E-WB (Panel B) assays for Fab7816 on carotid plaque lysate. Sample form spots C1 and C2 were used for the identification of the self-antigen protein.

FIG. 21. Panel A) Western-blotting of human transgelin with purified human recombinant Fabs. Panel B) Western-blotting of purified OmpK36 with purified human recombinant Fabs.

FIG. 22. Western blotting of OmpK36 with commercial anti-TAGLN antibodies. Upper panel: M06A (5 μg/mL and 1 μg/mL; M03 (5 μg/mL and 1 μg/mL); MO2 (5 μg/mL and 1 μg/mL); MO1 (5 μg/mL and 1 μg/mL: Lower panel M05 (5 μg/mL and 1 μg/mL); M04 (5 μg/mL and 1 μg/mL); negative control α-Apo B.

FIG. 23. OD measured by ELISA assay on purified OmpK36 bound by recombinant antibodies of the invention (panel A) or commercial anti-TAGLN monoclonal antibodies (panel B).

FIG. 24. panel A) Anti-OmpK36 antibody detection in patient sera, by ELISA on coated antigen. All the 5 tested sera show a specific positive response to OmpK36. panel B) Anti-TAGLN antibody detection in patient sera, by ELISA on coated antigen.

FIG. 25 The sequence listing submitted in parent application U.S. Ser. No. 12/679,109. SEQ ID NOS: indicated in italics in the specification refer to this sequence listing.

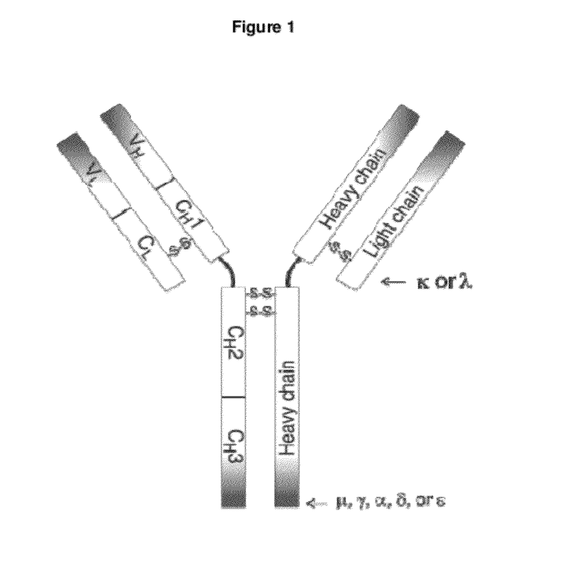

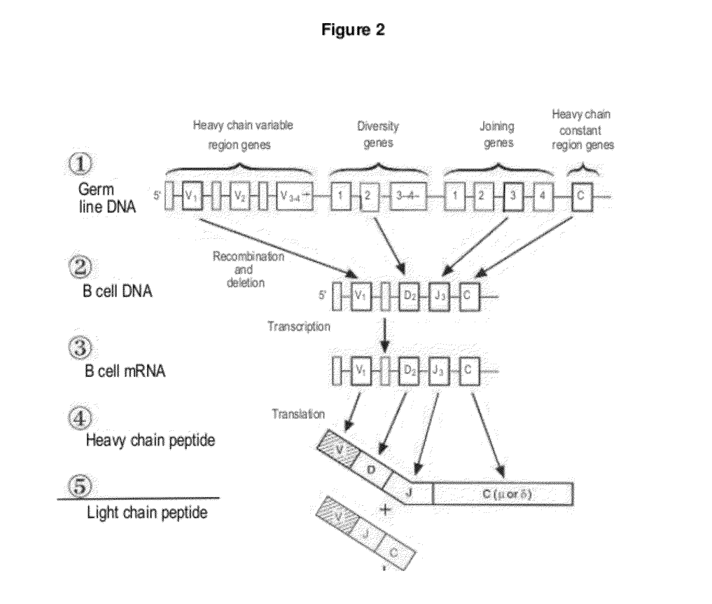

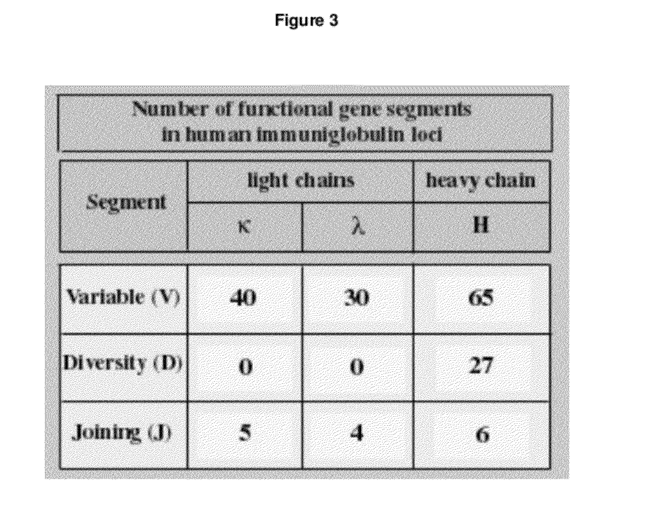

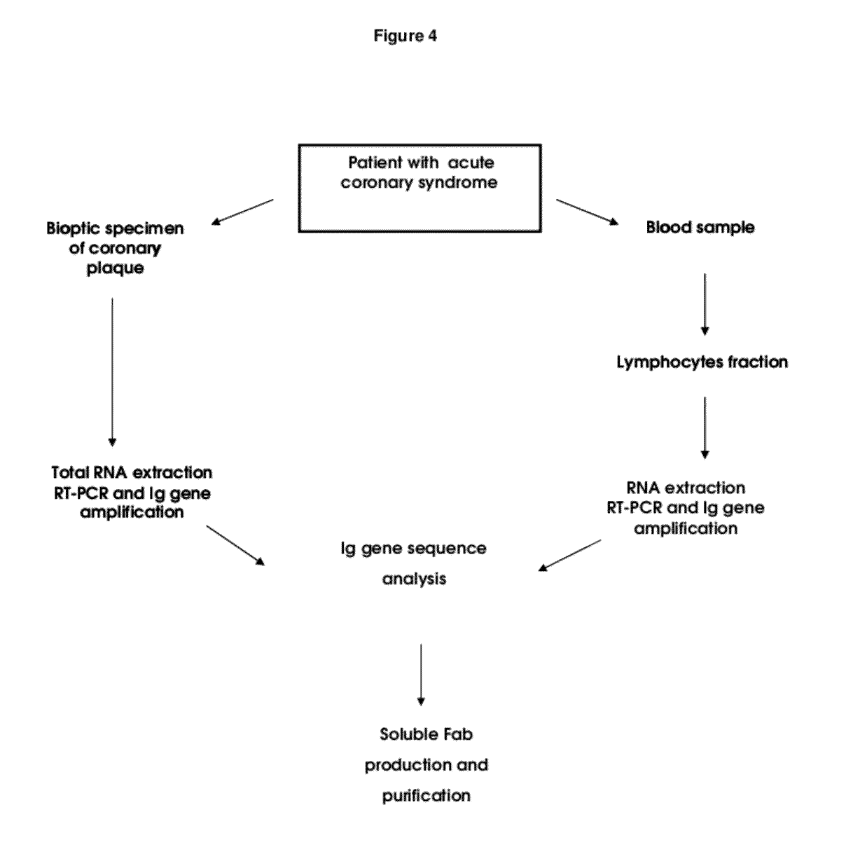

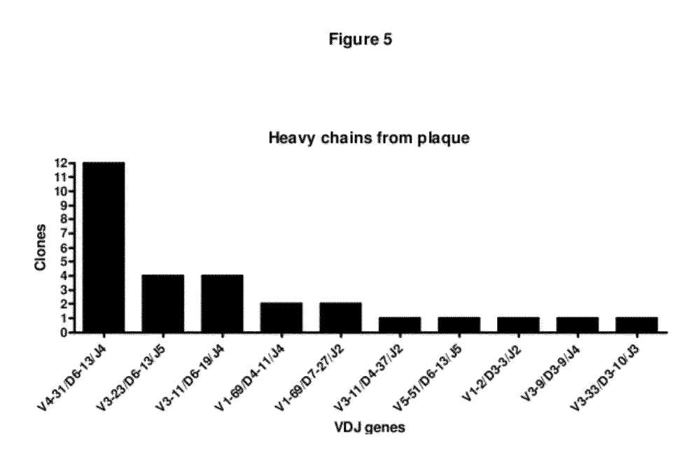

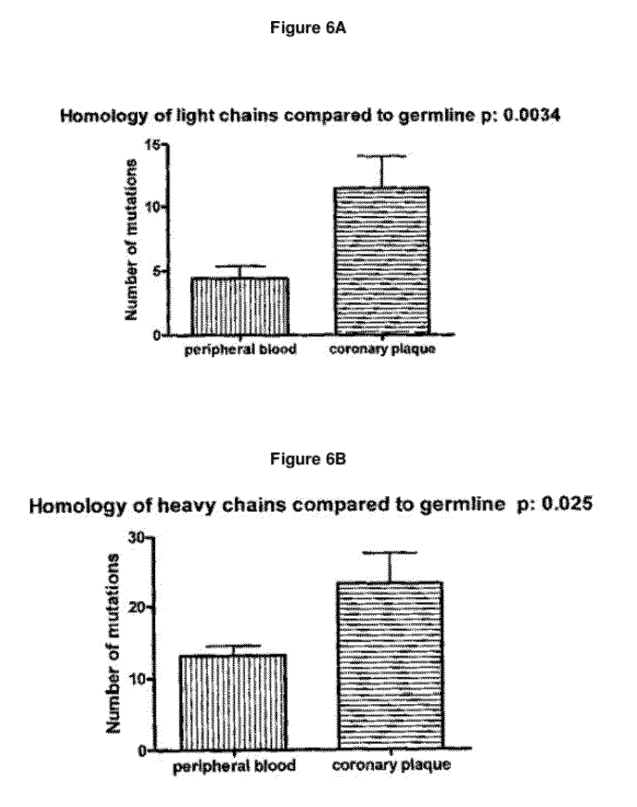

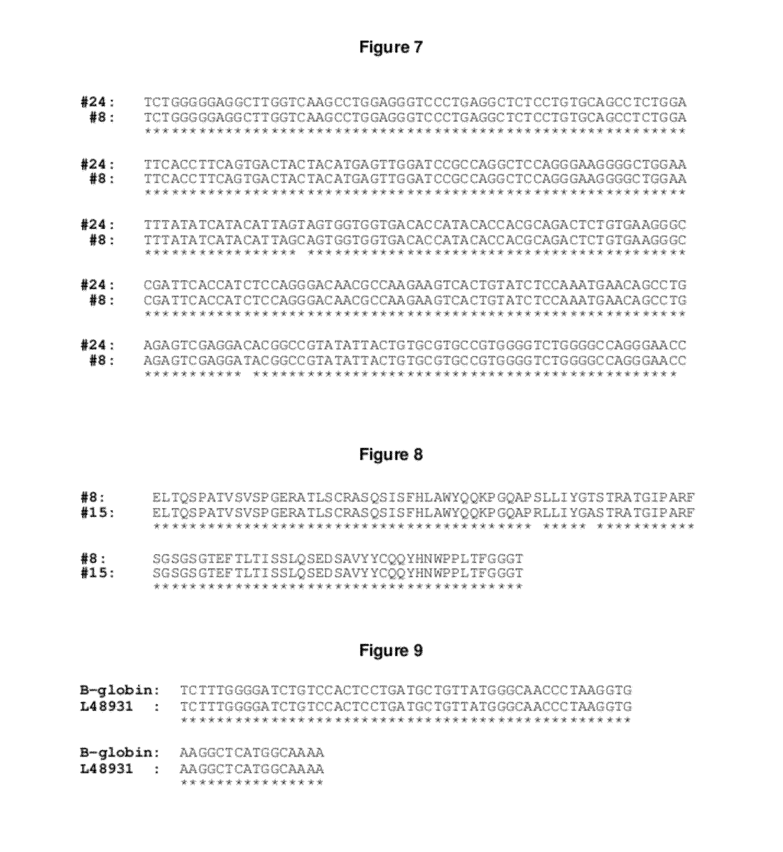

### DEFINITIONS

In the present invention, and unless otherwise provided, the term “isolated polynucleotide” or “isolated nucleotide” refers to a polynucleotide molecule, wherein polynucleotidic and nucleotidic, respectively, and polynucleotide and nucleotide are used alternatively with the same meaning, which is substantially free of any other cellular material or component that normally is present or interacts with it in its naturally occurring environment, such as fragments of other nucleotidic or polynucleotidic sequences, proteins or other cellular component.

Unless otherwise provided, “complementary sequence” refers to the sequence which hybridizes with the sequence of interest under stringent conditions, resulting in two hydrogen bonds formed between adenine and thymine residues or three hydrogen bonds formed between cytosine and guanine residues, respectively, and conservative analogs thereof having degenerative codon substitution or silent substitution, i.e. substitution of one or two or three consecutive nucleotides resulting in the same amino acid being coded due to the degeneracy of the genetic code.

The isolated polynucleotides within the meaning of the present invention, comprise, for instance, gene or gene fragments, exons, introns, mRNA, tRNA, rRNA, rybozyme, cDNA, plasmids, vectors, isolated DNA, probes and primers.

Unless otherwise indicated, the isolated polynucleotides of the invention, in addition to the specific ones described above, also comprise the complementary sequences thereto.

“cDNA” refers to the complementary DNA sequence, both single and double stranded and to any homologous sequence thereto and any fragment thereof, which codes continuously for an amino acidic sequence, i.e. its sequence is deprived of introns, and may be synthesized from isolated mRNA by retro-transcription techniques.

“Homologous sequence” within the meaning of the present invention refers to any sequence which is partially or almost identical to the sequence of interest; therefore, “homology” or “identity” of two or more sequences, comes from the factual measurement of the number of the same units, whether nucleotides or amino acids, out of the total units componing said nucleotide/amino acid sequence, which occupy the same position. For example, 90% homology means that 90 of every 100 units making up a sequence are identical when the two sequences are aligned for maximum matching. Within the present invention, homologous sequences have an identity of at least 85%, preferably of 90%, more preferably of 95% and even more preferably of at least 99.5%.

“Conservative substitution” of an amino acid is intended to be a substitution of an amino acid with another amino acid having the same properties, so that the substitution has no impact on the overall characterizing properties or functions of the peptide. Examples of such conservative substitutions include the substitution of an amino acid with another amino acid belonging to the same group as follows:
(i) amino acids bearing a charged group, comprising Glutamine and Aspartic acid, Lysine, Arginine and Histidine;
(ii) amino acids bearing a positively-charged group, comprising Lysine, Arginine and Histidine;
(iii) amino acids bearing negatively-charged group, comprising Glutamine and Aspartic acid;
(iv) amino acids bearing an aromatic group, comprising Phenylalanine, Tyrosine and Tryptophan;
(v) amino acids bearing a nitrogen ring group, comprising Histidine and Tryptophan;
(vi) amino acids bearing a large aliphatic nonpolar group, comprising Valine, Leucine and Isoleucine;
(vii) amino acids bearing a slightly-polar group, comprising Methionine and Cysteine;
(viii) amino acids bearing a small-residue group, comprising Serine, Threonine, Aspartic acid, Asparagine, Glycine, Alanine, Glutamic acid, Glutamine and Proline;
(ix) amino acids bearing an aliphatic group comprising Valine, Leucine, Isoleucine, Metionine and Cysteine;
(x) amino acids bearing a small hydroxyl group comprising Serine and Threonine.

In the following disclosure, “CDR3” refers to the complementary-determining region, which is formed by the junctions between the V-D-J gene (in the heavy chain) or V-J gene (in the light chain) segments coding for an antibody. CDR3 is found in the variable domains that complements an antigen.

“Single clone” refers to a sequence coding for the CDR3 region of an antibody, which is able to specifically bind an antigen/epitope.

Sequences showing the same CDR3 are deemed to be produced by the same clone.

“Clonal variant” is intended to be any sequence, which differs by one or more nucleotides or amino acids, in presence of V region with identical mutations compared to the germline, identical VDJ or VJ gene usage, and identical D and J length.

“Replacement mutation” is intended to be a nucleotidic mutation which causes another amino acidic to be coded.

“Silent mutation” is intended to be a nucleotidic mutation which does not cause a change in the coded amino acid due to the degeneracy of the DNA.

An “expression vector” is intended to be any nucleotidic molecule used to transport genetic information.

An “isolated expression system” is intended to be a system for the expression of amino acid molecules, and shall include one or more expression vectors comprising the nucleotidic sequences coding for one or more of the amino acid molecules of the invention and a suitable host cell in which the one or more vectors are transfected.

“Host cell” as for the present invention is intended to be a cell comprising one or more expression vectors of the invention and which is capable of producing the corresponding coded amino acid sequence or sequences or any fragments thereof, for example by expressing it on its surface.

“Antibodies” and “antibodies fragments” according to the present invention are intended to include whole antibodies, also referred to as immunoglobulin, of either type IgG, IgA, IgD, IgM or IgE, as well as any fragments thereof, such as proteolytic and/or recombinant fragments, like Fab fragments (produced upon digestion of Ig with papain), F(ab′)₂ (produced upon digestion of immunoglobulin with pepsin), Fab′, Fv, single chain antibodies (scFv) and single chain of antibodies, such as, for instance, heavy or light single chains.

“Ligand” within the present invention, is intended to be any agent that binds a recognized functional region of the antibody of the present invention or to any fragment thereof.

“Oligopeptide” according to the present invention is an amino acid sequence comprising less than 50 amino acid residues.

In the following description and unless otherwise provided, the “germline” sequence is intended to be the sequence coding for the antibody/immunoglobulin (or of any fragment thereof) deprived of mutations, therefore, the percentage of homology represents an indication of the mutational events which any type of heavy chain portion undergoes after contact with an antigen; more in particular, said mutations involve the CDR3 portion of the antibody/immunoglobulin or of any fragment thereof.

The “R:S mutation” ratio refers to the ratio between replacing (R) and silent (S) mutations occurring in the FR or CDR3 portion of the antibody/immunoglobulin coding sequence.

Said ratio is higher for CDR3 than that of the FR sequence, as CDR3 undergoes an higher number of mutational event in order to adapt to the structure of the antigen. FR, in contrast, is a more conservative sequence, generally.

### P-Value

“P-value” represents the significance of a mutational event.

In particular, the process of somatic hypermutation of rearranged V segments and the antigen selection of mutants with a higher affinity, allow the affinity maturation, in order to generate antibodies with improved binding properties to the antigen. This process leads to an accumulation of replacement mutations (R) in CDR regions, which are directly involved in the binding of antigen. On the contrary the silent mutations (S) accumulate in the FR regions, which are usually more conservative sequences in order to maintain the conformation of the antibody. In the absence of the antigen selection, a random mutational process results in random distribution of R and S mutations in the sequence of both heavy and light chains of an antibody. However during the selection process, the R:S mutation ratio for CDR3 is usually higher than that of the FR sequence.

Therefore, the p-value, which is calculated by multinomial distribution model that the excess (as for CDR) or the scarcity (as for FR) of mutations occurred by chance, relates to the probability of an antigen selection process. A low p-value indicates that there is a high probability that the variability of the heavy and light chains compared to the corresponding germline sequence, is due to the antigen-driven affinity maturation of the antibody.

A significant p-value is intended to be below 5%.

“Lineage trees” are a useful approach to study somatic hypermutation in B cell differentiation pathways by molecular analysis of antibodies genes expressed by clonally related cells.

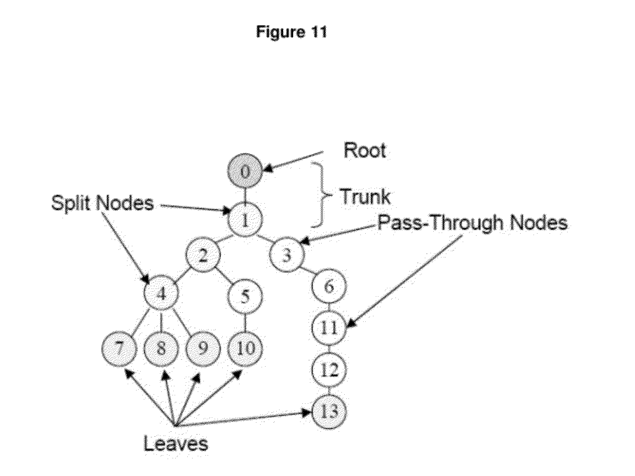

A lineage tree is defined, graphically, as a rooted tree where the nodes correspond to B cell receptor gene sequences (FIG. 11). For two nodes a and b it is said that b is a child of a if the sequence corresponding to b is a mutant of the sequence corresponding to a, which differs from b by at least one mutation and is one mutation further than b away from the original germline gene. Two B cells with identical receptors will correspond to the same node. Nodes in the tree can be either the root node, leaves (end-point sequences) or internal nodes, which can be either split nodes (branching points) or pass-through nodes.

Root is intended as representing the original B cell.

Leaves are intended to represent mutant B cells which were alive at the time of sampling and had no descendants at the time of observation.

Internal split nodes are intended as B cells that were produced during the maturation process and have more than one descendant.

Internal pass-through nodes refer to B cells with exactly one child.

Trunk is intended as the distance between the root to the first split node.

According to its first embodiment, the present invention concerns polynucleotidic molecules comprising any one of the sequences corresponding to the odd-numbered Sequence ID from 1 to 389 and from 395 to 453 and the complementary and homologous sequences thereto.

The polynucleotidic sequences of the present invention code for the amino acidic sequences of antibodies or any fragments thereof which bind to an antigen or any fragment thereof possibly found in the coronary plaque.

Preferably, within the present invention, the isolated polynucleotides of the above first embodiment are cDNA molecules.

cDNA is obtained by retro-transcription from mRNA molecules according to the well-known procedures in the art.

According to the first object of the present invention, there are also provided amino acid sequences corresponding to the even-numbered Sequence ID from 2 to 390 and from 396 to 454; as well as the homologous sequences thereof, and any sequences bearing conservative substitutions and fragments thereof.

As indicated, these definitions are intended to encompass analogous sequences, so as to include those sequences wherein, in the case of amino acid sequences, at least one or more amino acids are substituted by a derivative, such as the corresponding D-isomer or, for example, the corresponding sulphated, glycosylated or methylated amino acid; or one or more and up to 10% of the total amino acids making up a sequence may be substituted by a derivative thereof, such as, for example, cysteine may be substituted by homocysteine. There are also included sequences bearing conservative substitutions.

According to the present invention, there are also included the polynucleotidic sequences coding for antibodies or for any fragments thereof according to the first embodiment of the invention and having homology of at least 80%, preferably of at least 90%, more preferably of at least 95% and even more preferably of at least of 97% compared to the germline, when using a database available in ImMunoGeneTics (available through the web site http://imgt.cines.fr).

In addition, as for the first object of the present invention, hypermutated amino acid sequences are also encompassed.

Accordingly, there are also included the polynucleotide sequences coding for the amino acid sequences having a ρ-value of the CDR3 portion less than 5%, preferably less than 2%, more preferably less than 1% and even more preferably less than 1%o and the coded amino acidic sequences thereof.

As set hereinbefore, according to the present invention, there is included the synthesis of cDNA molecules, which is performed from mRNA isolated from a suitable sample of the active coronary plaque of a patient.

For the purpose of the present invention, said suitable samples of the active coronary plaque include a sample of the coronary plaque taken immediately after an infarction event, i.e. so-called “fresh-sample” or, alternatively, a sample may be taken and conserved under liquid nitrogen for a suitable period of time so as not to impair nor alter its histological properties and be further analysed.

For the purpose of the present invention, patients with acute coronary syndrome (ACS) have been selected for having experienced typical chest pain occurring less than 48 hours from hospital admission or ECG changes suggesting myocardial damage. In order to exclude possible confusing factors, patients with recent infectious diseases, immunologic disorders, immunosuppressive therapy or neoplastic diseases have been excluded.

Isolation of mRNA molecules from the above suitable samples, i.e. both from coronary plaque and peripheral blood, is carried out according to well-known methods. For a general reference, see, for instance Molecular cloning. Sambrook and Russell. Cold Spring Harbor Laboratory Press Cold Spring Harbor, N.Y. Third Edition 2001.

According to the second embodiment, the expression vector of the invention is selected from the group comprising for example, plasmid, cosmid, YAC, viral particle, or phage and comprises one or more of the polynucleotidic sequences according to the first embodiment of the invention; in a preferred aspect, the expression vector is a plasmid, comprising one or more of the polynucleotide sequences according to the first embodiment of the invention.

In a most preferred embodiment of the invention, the expression vector, i.e. a plasmid, comprises one or more of the polynucleotidic sequences of the invention selected from the group comprising the odd-numbered Sequence ID from 1 to 389 and from 395 to 453.

Expression vectors ordinarily also include an origin of replication, operably linked, i.e. connected thereto in such a way as to permit the expression of the nucleic acid sequence when introduced into a cell, a promoter located upstream of the coding sequences, together with a ribosome binding side, an RNA splice site, a polyadenylation site and a transcriptional sequence. The skilled artisan will be able to construct a proper expression vector and, therefore, any proper expression vector according to the selected host cell; for example, by selecting a promoter which is recognized by the host organism.

In an even more preferred embodiment, the expression vector of the present invention is represented by the vector described by Burioni et al. (Human Antibodies 2001; 10 (3-4): 149-54).

The isolated expression system according to the third embodiment of the invention may comprise a single expression vector, which comprises one or more of any one of the polynucleotidic sequences of the invention.

Alternatively, the above expression system may comprise two or more expression vectors, each of them comprising one or more of any one of the polynucleotidic molecules of the invention.

For example, an expression vector may comprise a polynucleotidic molecule of the invention coding for the light chain of an antibody or fragment thereof and a second expression vector may include a polynucleotidic molecule of the invention coding for the heavy chain of an antibody or fragment thereof.

In an embodiment of the invention, the expression system comprises a single expression vector including one or more of the polynucleotidic molecules comprising the odd-numbered Sequence ID from 1 to 389 and from 395 to 453 and coding for the amino acidic sequences and corresponded to the even-numbered Sequence ID from 2 to 390 and from 396 to 454 and any homologous sequence thereto.

In a preferred embodiment of the invention, the expression system comprises one expression vector comprising the polynucleotidic sequences coding for a light chain, i.e. being selected from the sequences corresponding to the odd-numbered Sequence ID from 53 to 63, 107 to 189, 211 to 251, 297 to 343, 351 to 369, 385 to 389 and from 429 to 453 and a second polynucleotidic sequence coding for a heavy chain, i.e. being selected from the sequences corresponding to the odd-numbered Sequence ID from 1 to 51, 65 to 105, 191 to 209, 253 to 295, 345 to 349, 371 to 383 and from 395 to 427.

In a most preferred embodiment of the present invention, the expression system includes a vector comprising the polynucleotidic sequence coding for the light chain as set forth in Sequence ID No 53 and the second vector comprising any one of the polynucleotide sequences coding for the heavy chain as set forth in Sequence ID No 21, 37, 43 and 51, respectively.

The preparation of the expression vector of the expression system of the invention, includes the insertion of the appropriate nucleic acid molecule or molecules into one or more vector or vectors, which generally comprises one or more signal sequences, origins of replication, one or more marker genes or sequences, enhancer elements, promoters, and transcription termination sequences according to methods well-known in the art.

For a general reference to said procedure, see, for instance Phage display, Cold Spring Harbor Laboratory Press. Cold Spring Harbor, N.Y.

For instance, the sequences coding for the heavy chain of the present invention are inserted into the expression vector with a Flag o a six-Histidine tail, for being easily detectable.

The host cell according to a fourth embodiment of the present may be, for instance, a prokaryotic cell or a eukaryotic cells.

Suitable prokaryotic cells include gram negative and gram positive and may include, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces. For example, publicly available strains which may be used are, for instance, E. coli K12 strain MM294 (ATCC 31,446); E. coli X1776 (ATCC 31,537); E. coli strain W3110 (ATCC 27,325) and K5 772 (ATCC 53,635) or E. coli XL1-Blue, which represents the preferred E. coli strain.

In addition to prokaryotes, eukaryotic microbes such as filamentous fungi or yeast are suitable host cells. Saccharomyces cerevisiae, also known as common baker's yeast, is commonly used; other yeast are, for instance, Saccharomyces, Pichia pastoris, or Kluyveromyces such as, for example, K. lactis, K. fragilis, K. bulgaricus, K. wickeramii, K. waltii, K. drosophilarum, K. thermotolerans, and K. marxianus, Schizosaccharomyces, such as Schizosaccharomyces pombe, yarrowia, Hansenula, Trichoderma reesia, Neurospora crassa, Schwanniomyces such as Schwanniomyces occidentalis, Neurospora, Penicillium, Tolypociadium, Aspergillus such as A. nidulans, Candida, Torulopsis and Rhodotorula.

In addition, suitable eukaryotic cells used for the preparation of the expression system may be derived from multicellular organisms as well, such as from invertebrate cells or plant cells. Plant cells include, for instance, Agrobacterium tumefaciens and Nicotiana tabacum. In addition, insect cells may be used, which include, for instance, Drosophila S2 and Spodoptera Sf9.

Conversely, mammalian host cell include Chinese hamster ovary (CHO) and COS cells. More specific examples further include monkey kidney CVI line transformed by SV40 (COS-7, ATCC CRL 1651); human embryonic kidney line, Chinese hamster ovary cells/-DHFR, mouse sertoli cells, human lung cells (W138, ATCC CCL 75); human liver cells (Hep G2, HB 8065); and mouse mammary tumor (MMT 060562, ATCC CCL51).

The selection of the appropriate host cell is deemed to be within the knowledge of the skilled person in the art, i.e. prokaryotic cells may be used for the preparation of antibody fragments such as Fabs, while for the preparation of whole antibodies such as IgG, eukaryotic cells like yeasts may be employed.

Methods for cell transfection and transformation in order to prepare the above disclosed host cell comprising the above expression system depends upon the host cell used and are known to the ordinarily skilled artisan.

For example, treatments with calcium or electroporation are generally used for prokaryotes, while infection with Agrobacterium tumefaciens is used for transformation of certain plant cells. For mammalian cells, calcium phosphate precipitation may be used as disclosed by Graham and van der Eb, Virology, 52:456-457 (1978).

However, other methods for introducing polynucleotidic sequences into cells, such as, for example, nuclear microinjection, electroporation, bacterial fusion with intact cells, or polycations, may also be used.

Host cells, in addition, may also be transplanted into an animal so as to produce transgenic non-human animal useful for the preparation of humanized antibodies or fragments thereof. A preferred non-human animal includes, for instance, mouse, rat, rabbit, hamster.

The production of recombinant antibodies and fragments thereof as for the fifth embodiment of the invention is performed according to known methods in the art and includes the use of the isolated polynucleotidic sequences of the invention.

In particular, said method includes the steps of:
a) isolating mRNA from a suitable sample of the coronary plaque;
b) performing reverse transcription in order to obtain the corresponding cDNA;
c) preparing an expression system comprising the one or more cDNA molecule or molecules obtained from step b) and any one of the above disclosed suitable host cells;
d) culturing the host cell under suitable growth conditions;
e) recovering the produced antibodies or any fragments thereof; and
f) purifying said antibodies or any fragments thereof.

In particular, steps a) to f) are performed according to known methods in the art as will be apparent from the following Examples.

In order to assess the influence on the results obtained by statistically occurring mutations or other mechanism different from those involved in the maturation of B-cells of the coronary plaque, cloning and sequencing is also performed on a small portion of a gene having a conserved region. Accordingly, as internal reference, β-globin gene is chosen; in particular, standard β-globin L48931 is used.

Therefore, it is a further object of the present invention, that the isolated recombinant antibodies and fragments thereof produced by the host cell of the present invention and according to the method disclosed above, include immunoglobulin (referred to as Ig) of the IgG type, while “fragments thereof” preferably include Fab fragments of IgG.

Preferably, the isolated recombinant antibody fragments of IgG of the present invention comprise the amino acid sequences set forth in Sequence ID No 54 and, alternatively, any one of the amino acid sequences set forth in Sequence ID No 22, 44, 52 and 38.

According to the present invention, there are also included the amino acid sequences coding for antibodies or for any fragments thereof which may be produced according to the process above disclosed and having homology of at least 80%, preferably of at least 90%, more preferably of at least 95% and even more preferably of at least of 97% compared to the germline, when using a database available in ImMunoGeneTics (available through the web site http://imgt.cines.fr).

In addition, there are also included the amino acidic sequences having a ρ-value of the CDR3 portion less than 5%, preferably less than 2%, more preferably less than 1% and even more preferably less than 1‰.

According to another object of the invention, there is provided an immunoassay, which comprises the use of the antibodies or of any fragments thereof produced according to the present invention.

Immunoassays are tests based on the formation of an antigen/antibody complex and can be either competitive or non-competitive.

Competitive immunoassays include the testing of unknown samples containing a particular antigen which competes for the binding to the antibodies with another, labelled antibody; therefore, the response is inversely proportional to the concentration of the antigen in the unknown sample.

Conversely, non-competitive immunoassays, also called “sandwich assays”, include the use of an immobilized antibody, bound by an antigen, thus forming a complex which is targeted by a labelled antibody; the result of said methods is, therefore, directly proportional to the concentration of the antigen.

Widely used immunoassays include, for example, RIA (Radio Immuno Assay), Western Blot, ELISA (Enzyme-linked Immunosorbent Assay), immunostaining, immunoprecipitation, immunoelectrophoresis, immunofluorescence, luminescent immunoassay (LIA), immunohystochemistry, which are routinely used in lab practise.

A preferred immunoassay according to the present invention is an ELISA test.

ELISA is a well-established biochemical technique, which allows the detection and further quantification of biomolecules, such as antibodies or fragments thereof, antigens, proteins, hormones and other organic molecules, in a given sample; preferably, according to the present invention, the above mentioned ELISA test is used for the detection of a specific antigen.

ELISA tests, in particular, may include the use of two antibodies, one of which, the first antibody, is selective for the molecule of interest, i.e. the antigen, and it is immobilized onto an ELISA plate. A mixture possibly containing said molecule of interest is added, incubation for a suitable and sufficient time is allowed, then a first washing is performed in order to remove unbound material. The secondary antibody coupled to an enzyme and specific for the complex formed between the molecule of interest and the first antibody is further added. There follows a second step of washing of the ELISA plate and the addition of a chromogenic substrate. The resulting variation in colour may be assessed by spectrophotometric techniques and is directly related through a colorimetric standard curve to the quantity of the complex formed and thus to the concentration of the molecule of interest present in the sample.

Samples to be tested by the above immunoassay of the invention are, for example, samples of the unstable coronary plaque taken from patient immediately after an infarction event, i.e. a so-called “fresh” sample as said before, or a sample which has been conserved under liquid nitrogen after being taken; alternatively, it may consist of a sample of whole blood or serum.

The immunoassay test according to the present invention represents a valuable diagnostic tool, when included in programs for the screening of either the population at risk or not of developing acute coronary syndrome (ACS) or other coronary diseases.

As for an additional embodiment of the invention, there is disclosed a therapeutic composition comprising an antibody or any fragment thereof of the present invention and a therapeutic moiety covalently linked thereto.

Said therapeutic composition is able to selectively target a therapeutic agent to the coronary plaque site.

Well-known advantages of said targeted composition include, among others, the possibility of reducing the quantity of active principle to be administered, thus reducing the potentially side effects thereof.

For said purpose, therapeutic moieties may include as non limiting examples, radionuclides, drugs, hormones, hormone antagonists, receptor antagonists, enzymes or proenzymes activated by another agent, autocrines or cytokines, antimicrobial agents; toxins can also be used.

Drugs and prodrugs are included as well.

A further embodiment of the invention relates to a diagnostic composition comprising an antibody of the invention or any fragment thereof linked to a diagnostic moiety for the visualisation of the coronary plaque site.

The diagnostic compositions according to the present invention comprise the antibody or any fragments thereof, produced according to the present invention, covalently linked to at least one diagnostic moiety in order to selectively target the coronary plaque site and thus allowing its localization.

Therefore, it will be possible to precisely localise the site where the coronary plaque developed and to even better understand the extent of the occurred lesion to the vase. In addition, this represents a very useful tool before removal of the plaque by surgery.

Diagnostic moieties allow the detection by the visualising techniques used in the field of medicine, such as, for example, MRI (magnetic resonance imaging), CT (computer tomography), ultrasound, ecography, x-rays, and other diagnostic techniques within the knowledge of the skilled person in the art.

The kind of diagnostic moiety will be selected according to the diagnostic technique to be used.

According to a still further object of the present invention, there are provided ligands, that is to say, molecules which bind selectively to the antibody or to any fragments thereof.

The ligand or ligands of the present invention may also be an agent that binds any surface or internal sequence or conformational domains or any other part of the target antibody or fragments thereof. Therefore, the “ligands” of the present invention encompass agents that may have no apparent biological function, beyond their ability to bind the target of interest.

Accordingly, proteins, peptides, polysaccharides, glycoproteins, hormones, receptors, cell surfaces antigens, antibodies or fragments thereof such as Fab fragments, F(ab′)2, Fab′, Fv and single chain antibodies (scFv) or even anti-idiotype antibodies, toxins, viruses, substrates, metabolites, transition state analogs, cofactors, inhibitors, drugs, dyes, nutrients, growth factors, etc., without limitation, are included as well within the above definition.

In a preferred embodiment, the ligand of the present invention is an oligopeptide as above defined; preferably is a peptide comprising 4 to 12 amino acids, more preferably is a peptide comprising 4 to 10 and even more preferably is a peptide comprising 6 to 8 amino acids.

The identification of the ligands may be performed by screening tests on libraries of compounds. In particular, according to the present invention, said identification includes the use of the antibodies provided by the present invention or of any fragments thereof.

A method for the identification of ligands to the antibodies of the present disclosure or to any fragments thereof, therefore, represents a further object of the invention.

For instance, said method may include the binding of the antibody or fragments thereof onto a solid phase, for example through a streptavidin-biotin linkage, followed by contacting the molecules to be tested with the thus prepared solid phase, so as to allow them binding to the complementary antibodies and then washing to remove unbounded material; finally, the extend of the binding can be determined by various methods such as, for instance, an ELISA test.

Preferably, said ELISA test is one wherein a first antibody or a fragment thereof, being selected from those of the present invention, is linked to a solid phase, for instance, by a biotin/streptavidin linkage, then a mixture containing the molecules to be tested is added, incubation is allowed for a suitable period of time, followed by removal of unbound material by washing. After that, the secondary antibody is admixed and incubation is allowed again. The molecules showing the highest affinity for the antibodies of the invention or for any fragments thereof may thus be isolated, identified and quantified according to well-known methods such as, for instance, by colorimetric measurements.

Alternatively, as for an additional embodiment of the present invention there is provided an immunoassay including the use of a ligand identified according to the present invention.

Said immunoassay may be any one of the immunoassays already mentioned above as for the second object of the invention.

For example, an immunoenzymatic test as for the claimed invention may be an immunohystologic assay as further detailed in Example 10.

The above immunohystologic assay can be performed in order to investigate the presence inside the plaque of the ligands identified and disclosed in the present invention according to the above embodiments.

In a still additional embodiment of the invention, there is disclosed a therapeutic composition comprising a ligand identified by the above method of the invention and covalently linked to a therapeutic moiety.

A therapeutic moiety for said purpose may be any one of those already described above.

In particular, the therapeutic composition thus provided may selectively target a therapeutic agent to the coronary plaque site.

There is also disclosed a diagnostic composition comprising a ligand identified by the above method of the invention and covalently linked to a diagnostic moiety.

A diagnostic moiety for said purpose may be any one of those already described above.

As for an additional embodiment of the invention, there is claimed the use of immunosuppressant compounds for the preparation of a pharmaceutical composition for the treatment of coronary diseases, such as the acute coronary syndrome (ACS) or of immuno-related pathologies.

Immuno-related pathologies include pathologies wherein the physiologic mechanisms triggering and controlling the immuno-responses are altered.

Immunosuppressant compounds may be selected from the group comprising by way of non limiting example, glucocorticoids, alkylating agents, antimetabolites, methotrexate, azathioprine and mercaptopurine, cytotoxic antibiotics such as dactinomycin, anthracyclines, mitomycin C, bleomycin, mithramycin, ciclosporine, interferons, opioids, TNF binding protein, mycophenolate, small biological agenst; in addition, monoclonal and polyclonal antibodies are comprised.

In a further embodiment, the present invention provides for a method for the identification, demonstration and characterization of a local antigen-specific and antigen- driven stimulation of the immune system, providing useful details that can be used for the identification of the aetiopatology, for the definition of targets and for the design of immunotherapy and immunoprophylaxis.

In particular, said method includes the steps of testing the affinity of the antibodies of the present invention or of any fragments thereof for pathogenic agents potentially responsible for the development of the coronary disease.

With the aim of better understanding of the present invention, and without posing any limitation to it, the following Examples are given.

### Experimental Section

### EXAMPLE 1

### Sample Collection

### 1a) Sampling of Atherosclerotic Coronary Plaque

A sufficient amount of tissue was obtained from an atherosclerotic plaque of a patient with acute coronary syndrome undergoing coronary atherectomy and it was stored in liquid nitrogen.

### 1b) Sampling of Peripheral Blood

5 ml of peripheral blood from the same patient from whom the tissue of Example la was taken, at the same time, and stored in tubes treated with EDTA.

### EXAMPLE 2

### mRNA Extraction

2a) mRNA Extraction from Coronary Plaque

The plaque taken according to Example la was homogenized and the total mRNA was extracted according to conventional methodologies using a commercial kit for the extraction of mRNA (Rneasy kit, Qiagen, Germany) and according to the instructions provided by the manufacturer.

2b) mRNA Extraction from Peripheral Blood Sample

5 ml of the peripheral blood collected according to Example lb was diluted in an equal volume of PBS (phosphate buffered saline) at 37° C., overlaid onto 15 ml of Histopaque-1077 (Sigma-Aldrich, St Louis, Mo.) and centrifuged at 300 g for 30 minutes at room temperature. Lymphocytes were collected at the interface using a Pasteur pipette, diluted in 15 ml of PBS and further centrifuged at 300 g. The obtained pellet was thus resuspended in 15 ml of PBS and a small aliquot is taken in order to count the cells using a counting chamber (Burker). Finally, the cell suspension was centrifuged at 300 g and mRNA extraction was performed on the obtained pellet according to the procedure described above.

### EXAMPLE 3

### mRNA Retrotranscription

3a) Retrotranscription of mRNA from Coronary Plaque Sample

Reverse transcription of mRNA from the coronary plaque sample obtained as from Example 2a was performed using a commercial kit for the retrotranscription of mRNA, Superscript III RT (Sigma-Aldrich, St Louis, Mo.) according to the manufacturer's instruction. The cDNA synthesis was performed according to standard procedures from the total mRNA primed with oligo(dT).

3b) Retrotranscription of mRNA from Peripheral Blood Sample

The same procedure of Example 3a was performed on mRNA obtained according to Example 2b.

### EXAMPLE 4

### Amplification of cDNA Sequences

4a) Amplification of cDNA Sequences from Coronary Plaque Sample

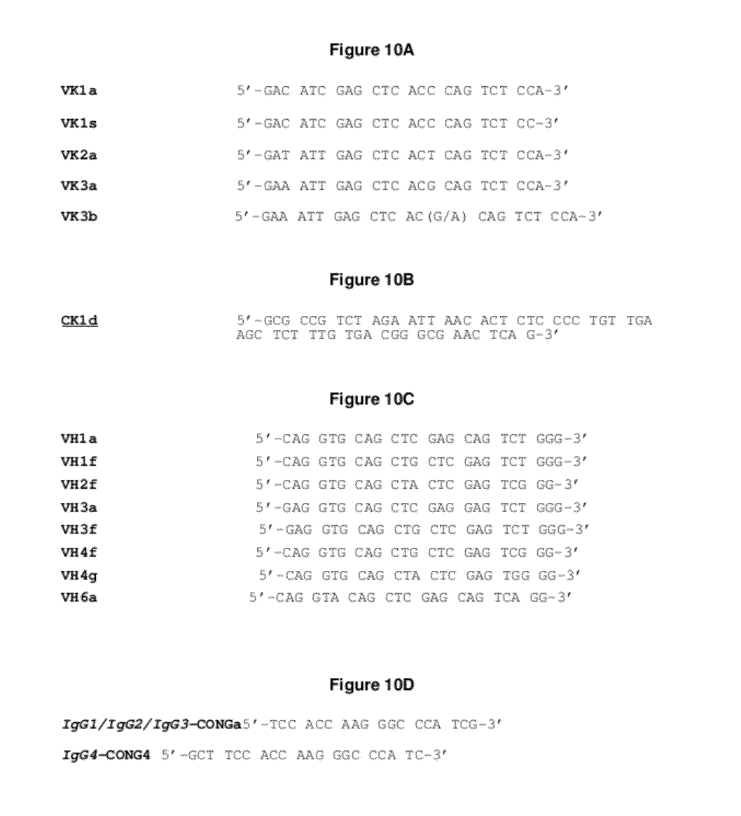

1 μl of cDNA obtained from the Example 3a underwent polymerase chain reaction. The reverse primers were designed in order to anneal to the segments of sequences coding for the constant region of heavy and light chains respectively (FIGS. 10B and D as for light and heavy chains, respectively). The forward primers are “family specific” and are designed to correspond to the 5′ end of the heavy and light chain genes in the first framework region FIGS. 10A and C as for light and heavy chains respectively); see, as a reference, Phage display, Cold Spring Harbor Laboratory Press. Cold Spring Harbor, N.Y. Third Edition 2001. For the heavy chains, primers specific for IgG1 and IgG2 isotypes were used, whereas for the light chains primers specific for K isotype were used. Amplification round was conducted with the following thermal profile: 94° C. for 15 seconds, 52° C. for 1 minute and 72° C. for 90 seconds. The reaction was conducted for 35 cycles. A negative control (the same mixture without DNA) and a positive control (a known sequence is inserted) were included in each reaction. The PCR product was analyzed by electrophoresis in a 2% agarose gel containing ethidium bromide. The reaction yields a ≅650 bp band corresponding to the light chains, and a 700 bp corresponding to the heavy chains. The amplicon, i.e. the product of the PRC process) was extracted from the gel with the use of a commercial kit for the extraction of DNA (QlAquick gel extraction kit; Qiagen, Germany) according to the manufacturer's instructions. Finally, the PCR products were recovered as per standard methods.

4b) Amplification of cDNA Sequences from Peripheral Blood Sample

The amplification of cDNA sequences from peripheral blood sample (cDNA obtained from Example 3b) was performed using the same procedure of Example 4a.

### EXAMPLE 5

### Sequencing

The sequences obtained according to the previous Examples were sequenced in for quantitative and qualitative analysis.

### 5.1) Heavy and Light Chain Sample Processing

A sample of clones of heavy and light chains obtained from coronary plaque sample and from peripheral blood sample obtained according to the previous Examples 4a and 4b, respectively, was picked up in order to be sequenced by Big Dye chemistry and analyzed using ABI PRISM 3100 sequencer.

The obtained sequences were individually aligned to the germline segments using a database available in ImMunoGeneTics (available through the web site http://imgt.cines.fr), in order to identify the V,D,J and V and J genes recurrence as for the heavy and light chains respectively, the homology level with the germline and the extent of somatic mutations. CDR3 sequence identity is used for identifying the clones; as mentioned above, sequences with identical CDR3 are deemed to come from the same clone.

The polynucleotide sequences from coronary plaque samples obtained according to the above Example 4a for the heavy chains correspond to the odd-numbered Sequence ID from 1 to 51, 65 to 105, 191 to 209, 253 to 295, 345 to 349, 371 to 383 and from 395 to 427 and code for the amino acidic sequences corresponding to the even-numbered Sequence ID from 2 to 52, 66 to 106, 192 to 210, 254 to 296, 346 to 350, 372 to 384 and from 396 to 428.

The polynucleotide sequences from coronary plaque samples obtained according to the above Example 4a for the light chains correspond to the odd-numbered Sequence ID from 53 to 63, 107 to 189, 211 to 251, 297 to 343, 351 to 369, 385 to 389 and from 429 to 453 and code for the amino acid sequences corresponding to the even-numbered Sequence ID from 54 to 64, 108 to 190, 212 to 252, 298 to 344, 352 to 370, 386 to 390 and from 430 to 454.

### 5.2) B-Globin Sequence: Internal Reference

The analysis of five clones showed that the obtained sequence of β-globin is identical to the sequence present in database (see FIG. 9, which reports one of the alignment with the standard β-globin L48931), thus demonstrating that no mutational event was due to the process variabilities.

5.3) Light Chains from Coronary Plaque Sample

The results of the sequencing of clones obtained from the coronary plaque samples according to Example 4a are shown in the following Table I for each clone V, D and J gene column report the type of sequence found to code for the V, D and J variable portion of the heavy chain, respectively. Homology percentage refers to the percentage of homology between each one of the sequence cloned from the coronary plaque sample and the sequence of the corresponding germline sequence as above disclosed.

TABLE I

| | | | | R:S | | p- | |
|---|---|---|---|---|---|---|---|
| | VK | JK | Homology | mutations | | value | |
| # Clone | gene | Gene | (%) | FR | CDR | FR | CDR |
| 1 | V3-15*01 | J4*01 | 96.86 | 1/0 | 4/2 | 0.00338 | 0.00063 |
| 2 | V3-15*01 | J4*01 | 96.07 | 2/1 | 4/2 | 0.00423 | 0.00207 |
| 7 | V3-15*01 | J4*01 | 96.07 | 2/1 | 4/2 | 0.00423 | 0.00207 |
| 15 | V3-15*01 | J4*01 | 96.07 | 2/1 | 4/2 | 0.00423 | 0.00207 |
| 24 | V3-15*01 | J4*01 | 96.07 | 2/1 | 4/2 | 0.00423 | 0.00207 |
| 25 | V3-15*01 | J4*01 | 96.07 | 2/1 | 4/2 | 0.00423 | 0.00207 |
| 38 | V3-15*01 | J4*01 | 96.07 | 2/1 | 4/2 | 0.00423 | 0.00207 |
| 67 | V3-15*01 | J4*01 | 96.07 | 2/1 | 4/2 | 0.00423 | 0.00207 |
| 86 | V3-15*01 | J4*01 | 96.07 | 2/1 | 4/2 | 0.00423 | 0.00207 |
| 36 | V3-15*01 | J4*01 | 96.07 | 2/1 | 4/2 | 0.00423 | 0.00207 |
| 8 | V3-15*01 | J4*01 | 94.90 | 3/2 | 5/2 | 0.00197 | 0.00091 |
| 32 | V3-15*01 | J4*01 | 95.31 | 3/2 | 4/2 | 0.00462 | 0.00489 |
| 10 | V3-15*01 | J4*01 | 94.90 | 3/2 | 5/2 | 0.00197 | 0.00091 |
| 29 | V3-15*01 | J4*01 | 94.90 | 3/2 | 5/2 | 0.00197 | 0.00091 |
| 39 | V3-15*01 | J4*01 | 94.90 | 3/2 | 5/2 | 0.00197 | 0.00091 |
| 9 | V3-15*01 | J4*01 | 95.22 | 2/1 | 5/3 | 0.00067 | 0.00061 |
| 28 | V3-15*01 | J4*01 | 95.45 | 4/2 | 1/3 | 0.045 | 0.3 |
| 51 | V3-20*01 | J4*01 | 86.77 | 24/4 | 2/1 | 0.89847 | 0.65339 |
| 52 | V3-20*01 | J4*01 | 86.77 | 24/4 | 2/1 | 0.89847 | 0.65339 |
| 57 | V3-20*01 | J4*01 | 86.77 | 24/4 | 2/1 | 0.89847 | 0.65339 |
| 63 | V3-20*01 | J4*01 | 86.77 | 24/4 | 2/1 | 0.89847 | 0.65339 |
| 49 | V1-33*01 | J4*01 | 94.9 | 3/4 | 3/0 | 0.00865 | 0.02608 |
| 53 | V1-33*01 | J4*01 | 94.9 | 3/4 | 3/0 | 0.00865 | 0.02608 |
| 56 | V1-33*01 | J4*01 | 94.9 | 3/4 | 3/0 | 0.00865 | 0.02608 |
| 64 | V1-33*01 | J4*01 | 94.9 | 3/4 | 3/0 | 0.00865 | 0.02608 |
| 29b | V3-11*01 | J2*01 | 95.68 | 3/2 | 2/0 | 0.10342 | 0.06186 |
| 58 | V5-2*01 | J1*01 | 97.25 | 5/0 | 1/0 | 0.74747 | 0.2472 |

5.4) Heavy Chains from Coronary Plaque Sample

The same procedure adopted for the analysis of the sequences of the light chains was repeated for the sequence of the heavy chains obtained according to Example 4a.

The results are shown in the following Table II.

TABLE II

| | | | | | R:S | | p- | | |
|---|---|---|---|---|---|---|---|---|---|
| | V | D | J | Homology | mutations | | value | | |
| #Clone | Gene | Gene | Gene | (%) | FR | CDR | FR | CDR | Isotype |
| 20(4) | V3-23*01 | D6-13*01 | J5*02 | 91.32 | 7/8 | 5/3 | 0.00131 | 0.11974 | IgG1 |
| 11/(2) | V3-23*01 | D3-10*01 | J3*02 | 95.07 | 4/3 | 4/2 | 0.01368 | 0.04852 | IgG2 |
| 13(12) | V4-31*03 | D6-13*01 | J4*02 | 93.58 | 8/4 | 5/0 | 0.12581 | 0.04459 | IgG1 |
| 9(4) | V3-11*01 | D6-19*01 | J4*02 | 92.07 | 11/6 | 4/0 | 0 | 0.23027 | IgG1 |
| 22(2) | V1-69*01 | D4-11*01 | J4*02 | 71.37 | 49/5 | 18/1 | 0.75964 | 0.00235 | IgG1 |
| 1 | V3-23*01 | D3-16*01 | J4*02 | 98.11 | 4/0 | 1/0 | 0.76745 | 0.31544 | IgG1 |
| 5 | V3-13*01 | D4-17*01 | J2*01 | 91.18 | 14/2 | 7/1 | 0.33946 | 0.01352 | IgG1 |
| 2 | V3-15*07 | D2-21*01 | J6*02 | 80.74 | 42/7 | 2/1 | 0.99879 | 0.99128 | IgG1 |
| 19 | V3-33*01 | D3-10*01 | J3*02 | 92.91 | 10/5 | 4/0 | 0.24167 | 0.19402 | IgG1 |
| 26 | V3-33*01 | D3-3*01 | J6*02 | 93.93 | 8/3 | 5/0 | 0.19861 | 0.03969 | IgG1 |
| 25 | V3-9*01 | D3-9*01 | J4*02 | 93.96 | 6/2 | 6/2 | 0.02766 | 0.00928 | IgG1 |
| 4 | V3-23*01 | D7-27*01 | J5*02 | 97.73 | 5/1 | 0/0 | 0.83827 | 0.78322 | IgG1 |
| 6 | V5-51*03 | D6-13*01 | J5*01 | 83.01 | 20/16 | 8/1 | 0.00469 | 0.18476 | IgG1 |
| 23 | V1-69*01 | D1-7*01 | J4*02 | 86.74 | 19/9 | 6/1 | 0.14441 | 0.20547 | IgG1 |

5.5) Light Chains from Peripheral Blood Sample

The same procedure applied for the analysis of the light chain as above disclosed was repeated on the sequences of the light chains obtained from the peripheral blood sample obtained according to Example 4b.

The results are shown in the following Table III.

TABLE III

| # Clone | VK Gene | JK Gene | Homology (%) | R:S mutations | |
|---|---|---|---|---|---|
| 4a | V4-1*01 | J1*01 | 98.16 | 4/1 | 0/0 |
| 5a | V4-1*01 | J1*01 | 95.6 | 3/4 | 2/0 |
| 9a | V4-1*01 | J4*01 | 98.16 | 1/0 | 3/0 |
| 7 | V3-20*01 | J2*01 | 98.44 | 1/0 | 1/1 |
| 8a | V3-20*01 | J2*02 | 96.12 | 0/1 | 4/1 |
| 10a | V3-20*01 | J2*01 | 98.44 | 2/0 | 0/1 |
| 2 | V3-20*01 | J1*01 | 100 | 0/0 | 0/0 |
| 14a | V3-20*01 | J1*01 | 96.51 | 3/2 | 2/0 |
| 1 | V3-15*01 | J4*01 | 97.64 | 1/2 | 3/1 |
| 1a | V3-15*01 | J1*01 | 94.5 | | |
| 12a | V3-15*01 | J2*01 | 100 | 0/0 | 0/0 |
| 5 | V5-2*01 | J2*01 | 95.68 | 7/3 | 0/0 |
| 6 | VD1-13*01 | J4*01 | 97.25 | 3/0 | 1/0 |
| 6a | V1-33*01 | J5*01 | 100 | 0/0 | 0/0 |
| 9a | V4-1*01 | J4*01 | 98.16 | 1/0 | 3/0 |
| 7a | V1-5*03 | J2*02 | 96.48 | 4/1 | 1/1 |
| 8 | V1-39*01 | J2*01 | 100 | 0/0 | 0/0 |
| 11 | V1-39*01 | J4*01 | 95.29 | 5/5 | 1/0 |
| 15 | V1-39*01 | J1*01 | 100 | 0/0 | 0/0 |
| 16 | V1-6*01 | J2*01 | 96.07 | 5/1 | 2/0 |
| 19 | V2-30*01 | J2*01 | 91.85 | 6/5 | 3/1 |
| 14 | V3-11*01 | J4*01 | 100 | 0/0 | 0/0 |
| 13 | V2-30*01 | J1*01 | 96.29 | 2/2 | 2/0 |

5.6) Heavy Chains from Peripheral Blood Sample

The same procedure was repeated on the sequences of the heavy chains from the peripheral blood sample and the results are shown in the following Table IV.

TABLE IV

| | | | | | R:S | |
|---|---|---|---|---|---|---|
| | VH | DH | JH | Homology | mutations | |
| # Clone | gene | gene | gene | (%) | FR | CDR |
| 5 | V4-59*02 | D6-25*01 | J3*02 | 88.93 | 13/6 | 7/3 |
| 8 | V3-48*01 | D3-3*01 | J4*02 | 93.18 | 7/7 | 4/0 |
| 12 | V3-23*01 | D3-10*01 | J3*01 | 91.69 | 11/2 | 7/2 |
| 14 | V4-34*01 | D2-2*01 | J6*02 | 96.94 | 1/4 | 3/0 |
| 18 | V4-34*01 | D3-22*01 | J1*01 | 96.18 | 6/2 | 2/0 |

Therefore, as clones 11, 9, 13 and 20 of the sequences amplified from the plaque show the highest divergence from the germline sequence, they are selected in order to be expressed together with the light chain 8.

### 5.7) Results

The above data show that both heavy and light chains from coronary plaque sample have an oligoclonal pattern and a characteristic VDJ and VJ gene pattern, respectively.

In addition, somatic hypermutations in the CDR3 portion are more frequent for the heavy and light chains of the coronary plaque sample compared to the peripheral blood sample; moreover, a higher number of mutational events occurred to the sequences of light and heavy chains from coronary plaque samples.

### 5.8) Mutational Lineage Tree

Lineage trees have been drawn for the sequences obtained according to the previous Examples aiming to illustrate diversification via somatic hypermutation of immunoglobulin variable-region (IGV) within clonally related groups of immunoglobulins.

### 5.8.1) Lineage Tree Generation

Germlines genes are identified according to Example 5. Tree bifurcations are identified by using a nj algorithm and the p model of evolution as implemented in the Mega 3 software (http://www.megasoftware.net/) using the germline sequence to root the tree. Manual corrections are performed to optimise the topology according to sequence visual inspection.

### 5.8.2) Results

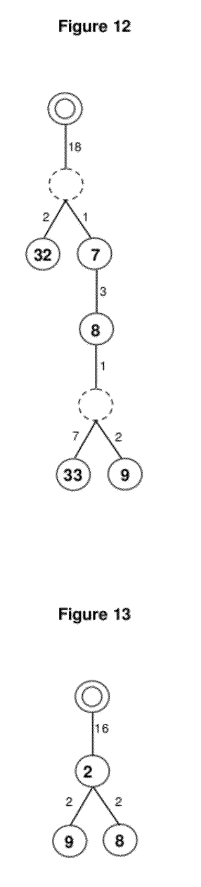

Results are shown in FIG. 12 and FIG. 13.

### EXAMPLE 6

### Preparation of the Expression System with Sequences from Coronary Plaque Sample and Transformation of Host Cells

Clones or light and heavy chain are then selected for transfection, in particular, clone 8 of the light chain (corresponding to Sequence ID No 53) and clones 11, 9, 13 and 20 of the heavy chains (corresponding to Sequence ID Nos 21, 43, 51 and 37, respectively) of the coronary plaque sample are selected to be transfected into the expression vector for the preparation of the soluble Fab fragments according to the following procedure.

Gene encoding for the light chains selected according to the above Example 6 and corresponding to Sequence ID No 53 is transferred into the expression vector pRB/expr and following the procedure disclosed by Burioni et al. Hum. Antibodies. 2001; 10(3-4):149-54.

Seq. ID No 53
GAGCTCACGCAGTCTCCAGCCACCGTGTCTGTGTCTCCAGGGGAAAGAGCCACCCTCTCCTG
CAGGGCCAGTCAGAGTATTAGTTTCCACTTAGCCTGGTACCAGCAGAAACCTGGCCAGGCTC
CCAGTCTCCTCATCTACGGAACATCCACCAGGGCCACTGGTATCCCAGCCAGGTTCAGTGGC
AGTGGGTCTGGGACAGAGTTCACTCTCACCATCAGCAGCCTGCAGTCTGAAGATTCTGCGGT
TTATTACTGTCAGCAGTATCATAACTGGCCTCCCCTCACTTTCGGCGGAGGGACC

In the expression vector comprising the gene coding for the selected light chain (clone 8 selected from Example 5) is further introduced the gene coding for the heavy chain corresponding to the clone 11 (corresponding to Sequence ID No 21) following the same procedure disclosed by Burioni et al. Hum Antibodies. 2001; 10(3-4):149-54.

Seq. ID No 21
CTCGAGTCTGGGGGAGGCTTGGGACAGCCTGGGGGGTCCCTGAGACTCTCCTGTGCAGCCTC
TGGATTTACCTTTAGCAGCTATGCCATGAGCTGGGTCCGCCAGGCTCCAGGGAAGGGGCTGG
AGTGGGTCTCAGCTATTAGTGATAGGGGGGAGAGCACATACTACGCAGACTCCGTGAAGGGC
CGGTTCACCATCTCCAGGGACAATTCTAAGAACACGCTGTATGTGCAAATGAACAGCCTGAG
AGCCGAGGACACGGCCCTATATTTCTGCGCGAAAGATCAATTTCTATGGTTCGGGGAGTCAA
CAGCGGGTGATGCTTTTGATATCTGGGGCCAAGGGACA

The expression vector is introduced into the E. coli XL-1 Blue for the expression of soluble Fabs.

In particular, 10 ml of SB (Super Broth, Becton, Dickinson, N.J.) with ampicillin (100 ng/ml, Sigma-Aldrich, St Louis, Mo.) and tetrayicline (10 ng/ml, Sigma-Aldrich, St Louis, Mo.) was inoculated with a single bacterial colony from a fresh plate and incubated overnight at 37° C. in an orbital shaker

After that, 2.5 ml of this culture was inoculated into 1 liter of SB/amp-tet (the above mixture of SB, ampicillin and tetracyclin) into a 5 liter flask and allowed to grow until an Optical Density (OD₆₀₀) of approximately 1.0. Then IPTG (isopropyl-beta-D-thiogalactopyranoside; Biorad, California) was added up to a final concentration of 1 mM and the bacterial cultures were incubated overnight at 30° C. in the orbital shaker. Thus, bacteria were centrifuged at 3000 rpm for 20 minutes at 4° C. and the pellets were resuspended in 10 ml PBS. Subsequently, 50 μl PMSF (from a stock solution of 100 mM) was added in order to inhibit the proteases and bacteria were sonicated three times in ice, 3 minutes for each run. The bacterial culture was centrifuged at 18000 rpm for 45 minutes at 4° C. and the supernatant is filtered carefully with a 0.22 μm diameter membrane (Millipore®). Meanwhile, the column was washed with 10 volumes of PBS and subsequently the filtered supernatant was added slowly to the column. After washing with at least 30 volumes of PBS, Fabs are eluted with 100 mM glycine/HCl pH 2.5. 10 fractions are collected (each one of about 1 ml) and immediately neutralized with Tris 1M pH 9.

Purified Fabs were tested in SDS-PAGE gel in non-reducing conditions showing a single band of approximately 50 kDa.

Fabs were quantified comparing the relative band with at least two different standard concentrations of BSA.

### EXAMPLE 7

### Preparation of the Expression System with Sequences from Atherosclerotic Plaque Sample and Transformation of Host Cells

The same procedure disclosed in Example 6 was repeated by introducing into the expression vector the gene for the light chain of clone 8 selected according to Example 5 and the sequence coding for the heavy chain of clone 9 (corresponding to Sequence ID No 43).

Seq ID No 43
CTCGAGTCTGGGGGAGGCTTGGTCAAGCCTGGAGGGTCCCTGAGGCTCTCCTGTGCAGCCTC
TGGATTCACCTTCAGTGACTACTACATGAGTTGGATCCGCCAGGCTCCAGGGAAGGGGCTGG
AATTTATATCATACATTAGTAGTGGTGGTGACACCATACACCACGCAGACTCTGTGAAGGGC
CGATTCACCATCTCCAGGGACAACGCCAAGAAGTCACTGTATCTCCAAATGAACAGCCTGAG
AGTCGAGGACACGGCCGTATATTACTGTGCGTGCCGTGGGGTCTGGGGCCAGGGAACC

### EXAMPLE 8

### Preparation of the Expression System with Sequences from Atherosclerotic Plaque Sample and Transformation of Host Cells

The same procedure disclosed in Example 6 was repeated by introducing into the expression vector the gene for the light chain of clone 8 selected according to Example 5 and the sequence coding for the heavy chain of clone 13 (corresponding to Sequence ID No 51).

Seq. ID No 51
CTCGAGTCGGGCCCAGGACTGGTGAAGCCTTCACAGACCCTGTCCCTCACCTGCACTGTCTC
TGGTGGCTCCATCAGCAGTGGTTACTACTGGACCTGGATCCGCCAGTACCCAGGGAGGGGCC
TGGAGTGGATTGGATACATCTCTTACAGGGGGAGCACCTACTACAACCCGTCCCTCAAGAGT
CGAGTTACAATATCACTAGACACGTCTAAGAACCAGTTTTTCTTGAACCTGACCTCTGTGAC
TGCCGCGGACACGGCCGTGTATTTCTGTGCGAGAGATCGGAGTAGAGCAACATCTGGTATTC
TTGACTACTGGGGCCAGGGAACC

### EXAMPLE 9

### Preparation of the Expression System with Sequences from Atherosclerotic Plaque Sample and Transformation of Host Cells

The same procedure disclosed in Example 6 was repeated by introducing into the expression vector the gene for the light chain of clone 8 selected according to Example 5 and the sequence coding for the heavy chain of clone 20 (corresponding to Sequence ID No 37).

Seq. ID No 37
CTCGAGTCGGGGGGAGGCTTCGTACAGCCTGGGGGGTCTCTGAGACTCTCCTGTGCAGCCTC
TGGATTCACCTTCAGGGACTATGCCATGGGCTGGGTCCGCCAGGCTCCAGGGAAGGGGCCGG
AGTGGGTCTCAATTATTAGTGCTAGTGGTGGTTCCATATACTACGCAGACTCCGTGAAGGGC
CGATTCACCATCTCCAGAGACAACGCCAAGAACACACTGTATCTGCAAATGAACAGTCTCAG
AGCCGACGACACGGCTGTATACTACTGTGCAAGACAGACCAGCAGCAGATGGTATGATTGGT
TCGACCCCTGGGGCCAGGGAACC

### EXAMPLE 10

### Immunohystologic Assay

A fresh sample of plaque was frozen in liquid nitrogen and sectioned using a cryostat. Sections 5 μm thick were fixed with ice-cold acetone and blocked with a serum blocking solution (2% serum, 1%BSA, 0.1% Triton X-100, 0.05% Tween 20) for 1 hour at room temperature. The fixed sections were probed with the Fabs produced and identified according to the present invention, at an appropriate dilution, and incubated for 2 hours at room temperature. Sections were washed five times with PBS and an appropriate dilution of a FITC (fluorescein isothiocyanate)-conjugated secondary anti-human Fab (Sigma-Aldrich, St Louis, Mo.) was added. After 30 minutes at room temperature, sections were washed again and the complex ligand/antibody thus formed was detected with a fluorescence microscope.

### EXAMPLE 11

### Antibody Screening of Phage Library

Panning of the random phage-displayed peptide library expressing dodecapeptides at the N-terminus of cpIII coat protein of the filamentous phage M13 (Ph.D.-12™ Phage Display Peptide Library Kit, Catalog #E8110S, New England Biolabs, Beverly, Mass.) was performed according to the manufacturer's instructions using Fab-coated high-binding 96-well ELISA plates (Costar 96w polystyrene ½ area flat bottom HI-binding flat bottom, cat #3690).

In order to remove phages binding to antibody conserved regions, a negative selection was performed from the second round of panning by combining the amplified phages with 25 μg of a pool of human standard IgG (Endobulin, A.T.C J06BA02, Baxter S.p.A.) for 1 hour at 37° C.

Four rounds of selection were performed as described above, panning the amplified phage on Fabs produced and identified according to the present invention and the same pool of standard IgG used for the negative selection.

### EXAMPLE 12

### Peptide Screening and DNA Sequence Analysis

All the phages obtained as from Example 11 were used to infect E. coli strain ER2537 and randomly picked single plaques were screened in enzyme-linked immunoassay on Fabs produced and identified according to the present invention and the pool of standard IgG.

Antigen-coated plates (Costar 96w polystyrene ½ area flat bottom HI-binding flat bottom, cat #3690) were washed and blocked with a solution of PBS/BSA 1% for 1 hour at 37° C.; 50 μl of 10⁸ phages per milliliter are added and incubated for 2 hours at 37° C.

Plates were washed 10 times with PBS (0.1% Tween-20; Sigma-Aldrich, St Louis, Mo.); afterward, 50 μl of a 1:3000 dilution in PBS of a HRP-conjugated anti-M13 antibody (GE Healthcare 27-9411-01) was added.

After 2 hours at 37° C. plates were washed with PBS (0.5% Tween-20; Sigma-Aldrich, St Louis, Mo.), specific bound phages were detected by adding 100 μl of substrate (Sigma-Aldrich, St Louis, Mo.) and plates were read for an Optical Density of 450 nm after 30 minutes at room temperature.

Positive clones showing an OD_{450 nm} value >1 on Fabs of the present invention and OD_{450 nm} value <0.3 on pool of IgG were scored as positives and evaluated by sequence analysis using the software Pepitope http://pepitope.tau.ac.il/index.html. From peptide sequence analisys conserved aminoacidic positions were identified and four peptides were selected on the basis of the amount of consensus residues present in their sequences.

Four peptides have been identified corresponding to Sequence ID from 391 to 394.

### EXAMPLE 13

### Enzyme-Linked ImmunoSorbent Assay

Hep-2 (ATCC CCL-23) cells were grown in E-Mem (Invitrogen 0820234DJ) supplemented with Antibiotic/Antimycotic Solution (Invitrogen, Antibiotic/Antimycotic Solution, liquid 15240-062) and 10%FBS. Cells were regularly split 1:10 every 5 days. Five million cells were washed in PBS and lysed by using RIPA buffer (50 mM Tris HCL ph8+150 mM NaCl+1% NP-40+0.5% NA deossicolate+0.1% SDS).

Elisa plates (Costar 96w polystyrene ½ area flat bottom HI-binding flat bottom, cat #3690) were coated with serial dilution of of Hep-2 Lysate (1000 ng, 200 ng, 40 ng and 8 ng in PBS) overnight at 4° . After blocking with PBS+BSA3% for 2 hours at 37° C., serial dilutions of Fab 24 (20 μg/ml, 10 μg/ml, 5 μg/ml, 2.5 μg/ml, were incubated with the coated antigens for 1 hour at 37° C. After washing with PBS +Tween20 0.1% (SIGMA cod. PL379), plates were incubated with anti human IgG peroxidase (SIGMA cod. A2290) for 30 minutes at 37° C. After washing with PBS+Tween 0.1%, TMB substrate was added to the wells (PIERCE TMB substrate kit for peroxidase cod. SK 4400). ELISA plates were analysed with a spectrophotometer at 450 nm.

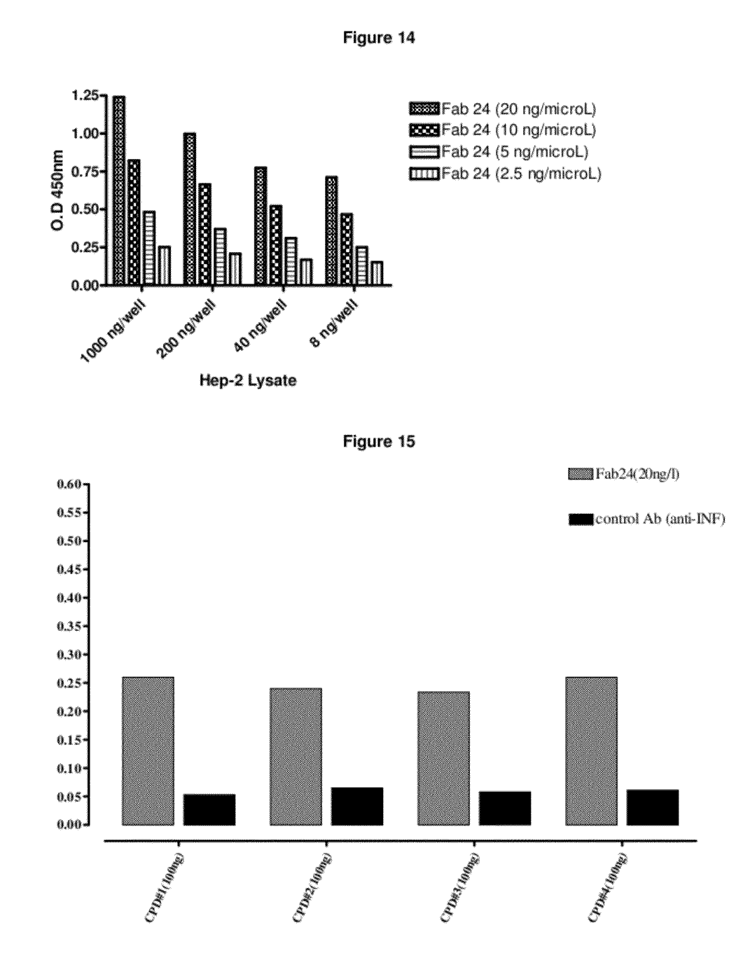

Results are shown in FIG. 14.

### EXAMPLE 14

### Synthesis of the Peptides

### 14.1) General

Abbreviations for Chemical Reagents, Chemical Structure Moieties and Techniques: AA—amino acid, AcOH—Acetic acid, ACN—Acetonitrile, API-ES—Atmospheric pressure ionization electrospray, Btn—Biotin, Boc—tert-Butyloxycarbonyl, DCM—Dichloromethane, DIC—N,N-Diisopropylcarbodiimide, DIEA—N,N-Diisopropylethylamine, DMF—N,N-Dimethylformamide, Et₂O—Diethyl ether, Fmoc-9-Fluorenylmethoxycarbonyl, Adoa—8-Amino-3,6-dioxaoctanoic acid, HFIP—1,1,1,3,3,3-hexafluoro-2-propanol, HOBt—N-Hydroxybenzotriazole, MeOH—Methanol, Neg. ion—Negative ion, NHS—N-Hydroxysuccinimide, NMP—N-Methylpyrrolidone, Pip—Piperidine, Pos. ion—Positive ion, HBTU—O-(Benzotriazol-1-yl)-N,N,N′,N′-tetramethyluronium hexafluorophosphate, PyBOP—Benzotriazole-1-yl-oxy-tris-pyrrolidino-phosphonium hexfluorophosphate, t_{R}—Retention time (minutes), Reagent B (88:5:5:2—TFA:H₂O:phenol:TIPS—v/v/wt/v), Su—Succinimidyl, TFA—Trifluoroacetic Acid, TIPS—Triisopropylsilane, H₂O—Water.

Names, structures and abbreviations used for amines and unnatural amino acids used in the synthesis of various peptides are given in Table V.

Solvents for reactions, chromatographic purification and HPLC analyses are E. Merck Omni grade solvents from VWR Corporation (West Chester, Pa.). NMP and DMF are purchased from Pharmco Products Inc. (Brookfield, Conn.), and are peptide synthesis grade or low water/amine-free Biotech grade quality. Piperidine (sequencing grade, redistilled 99+%) and TFA (spectrophotometric grade or sequencing grade) are purchased from Sigma-Aldrich Corporation (Milwaukee, Wis.) or from the Fluka Chemical Division of Sigma-Alrich Corporation. Phenol (99%), DIEA, DIC and TIPS are purchased from Sigma-Aldrich Corporation. Fmoc-protected amino acids, PyBop, and HOBt used are purchased from Nova-Biochem (San Diego, Calif., USA), Advanced ChemTech (Louisville, Ky., USA), Chem-Impex International (Wood Dale Ill., USA), and Multiple Peptide Systems (San Diego, Calif., USA). Fmoc-Adoa and Btn-Adoa-Adoa-OH are obtained from NeoMPS Corp (San Diego, Calif.).

Analytical HPLC data were generally obtained using a Shimadzu LC-10AT VP dual pump gradient system employing either Waters X-Terra® MS-C18 (5.0μ, 50×4.6 mm; 120 Å pore size) or Waters Sunfire™ OBD-C8 (4.6×50 mm 3.5μ, 120Å pore size) columns and gradient or isocratic elution systems using H₂O (0.1% TFA) as eluent A and ACN (0.1% TFA) as eluent B. Detection of compounds was accomplished using UV at 220 and/or 230 nm.

Preparative HPLC was conducted on a Shimadzu LC-8A dual pump gradient system equipped with a SPD-10AV UV detector fitted with a preparative flow cell. Generally the solution containing the crude peptide was loaded onto a reversed phase Waters Sunfire™ OBD C8 (50×250 mm; particle size: 10.0μ, 120 Å pore size) column, using a third pump attached to the preparative Shimadzu LC-8A dual pump gradient system. After the solution of the crude product mixture was applied to the preparative HPLC column the reaction solvents and solvents employed as diluents, such as DMF or DMSO, were eluted from the column at low organic phase composition. Then the desired product was eluted using a gradient elution of eluent B into eluent A. Product-containing fractions were combined based on their purity as determined by analytical HPLC and mass spectral analysis. The combined fractions were freeze-dried to provide the desired product.

Mass spectral data are obtained in-house on an Agilent LC-MSD 1100 Mass Spectrometer. For the purposes of fraction selection and characterization of the products, mass spectral values were usually obtained using API-ES in positive ion mode. Generally the molecular weight of the target peptides is ˜2000; the mass spectra usually exhibited strong doubly or triply positively charged ion mass values rather than weak [M+H]⁺. These were generally employed for selection of fractions for collection and combination to obtain the pure peptide from HPLC purification.

### 14.2) General Methods for Solid Phase Peptide Synthesis (SPPS)

14.2.1) The linear peptides were synthesized by an established automated protocol on a Rainin PTI Symphony® Peptide Synthesizer (twelve peptide sequences/synthesis) using Fmoc-Pal-Peg-PS resin (0.2 mmol/g) and/or suitably preloaded resins, Fmoc-protected amino acids and PyBop-mediated ester activation in DMF. The rest of the peptide sequence was loaded on the Fmoc-Pal-Peg-PS and/or other resins in stepwise fashion by SPPS methods typically on a 0.2 mmol scale. The amino acid coupling was carried out with a 4-fold excess each of amino acid activated by PyBop-DIEA reagent in DMF. Biotin is coupled to N-terminus of the peptide with a four-fold excess of Btn-NHS ester.

14.2.2) When preloaded diamines on trityl resins were used, after final acetylation the fully protected peptide chain was cleaved from the resin with 8:1:1—DCM: AcOH: HFIP and after evaporation of the volatiles, the final Btn-Adoa-Adoa was coupled to the amine at the C-terminus in solution. The crude fully protected peptide was treated with 1.0 equivalent of pre-formed Btn-Adoa-Adoa-NHS ester in solution for 16 h at RT (total volume 5.0 mL/g of the crude weight).

In a typical coupling process for a given amino acid, 6.0 mL of DMF solution containing 0.8 mmol of an amino acid followed by PyBOP (0.8 mmol, DMF solution, 1.25 mL) and DIEA (0.8 mmol, DMF solution, 1.25 mL) were added in succession by an automated protocol to a reaction vessel (RV) containing the resin (0.2 mmol) and the resin was agitated by recurrent nitrogen bubbling. After 1 hour the resin was washed thoroughly with DMF (4.5 mL, 6×) and the cleavage of the Fmoc-group was performed with 25% Pip in DMF (4.5 mL) for 10 minutes followed by a second treatment with the same reagent for 10 minutes to ensure complete deprotection. Again the resin was thoroughly washed with DMF (5 mL/g, 6×); a DCM (10 mL/g) wash was performed in between DMF washes to guarantee that the resin is freed of residual Pip. After completion of the peptide synthesis, the resin bearing the fully protected peptide was cleaved with, Reagent B (10.0 mL/g of resin or 10.0 mL/g of crude protected peptide) for 4 hours. The volatiles were removed under vacuum to provide a paste. The paste thus obtained was triturated with Et₂O to provide a solid which was pelleted by centrifugation followed by 3 more cycles of Et₂O washing and pelleting. The resulting solid was dried under vacuum to provide the crude peptide. A 200 μmol scale synthesis of a peptide of MW ˜2000 gave 200-300 mg (90-110% of theory) of the crude peptide. The greater than theoretical yield is most likely due to moisture and residual solvents.

### 14.3) Purification of Peptides—General Procedure

A 200 μmol scale synthesis of a peptide of MW ˜2000 on the ‘Symphony’ instrument provided ˜200-300 mg of crude peptide from each reaction vessel (RV). The crude peptide (˜200-500 mg) was purified in one run by reversed phase HPLC. The crude peptide (˜200 mg) dissolved in ACN (10 mL) was diluted to a final volume of 50 mL with H₂O and the solution was filtered. The filtered solution was loaded onto a preparative HPLC column (Waters, Sunfire™ Prep C8, 50×250 mm 10μ, 120 Å) which had been pre-equilibrated with 10% ACN in H₂O (0.1% TFA). During the application of the solution to the column the flow of the equilibrating eluent from the preparative HPLC system was stopped. After the solution was applied to the column, the flow of equilibrating eluent from the gradient HPLC system was reinitiated and the composition of the eluent was then ramped to 20% ACN-H₂O (0.1%TFA) over 10.0 minutes after which a linear gradient at a rate of 0.5%/min of ACN (0.1% TFA) into H₂O (0.1% TFA) was initiated and maintained for 60 minutes. Fractions (15 mL) were collected using UV at 220 nm as an indicator of product elution. The collected fractions were analyzed on an analytical reversed phase C8 column (Waters Sunfire™ OBD-C8, 4.6×50 mm, 5μ, 120 Å) and product-containing fractions of >95% purity were combined and freeze-dried to afford the corresponding peptide. After isolation, the peptides were analyzed by HPLC and mass spectrometry to confirm identity and purity. Data for the peptides is provided in Table VI (Sequence, Resin Loading and Yield), Table VII (HPLC and Mass Spectral Analysis) and Table VIII (Peptide Structures).

### EXAMPLE 15

### Enzyme-Linked ImmunoSorbent Assay

Elisa plates (Costar 96w polystyrene ½ area flat bottom HI-binding flat bottom, cat #3690) were coated with 100 ng of peptides resuspended in PBS overnight at 4° C. After blocking with PBS+BSA3% for 2 hours at 37° C., Fab 24 (20 μg/ml) was incubated with the coated antigens for 1 hour at 37° C. After washing with PBS+Tween20 0.1% (SIGMA cod: PL379), plates were incubated with anti human IgG peroxidase (SIGMA cod: A2290) for 30 minutes at 37° C. After washing with PBS+Tween 0.1%, TMB substrate was added to the wells (PIERCE TMB substrate kit for peroxidase cod: SK 4400). ELISA plates were analysed with a spectrophotometer at 450 nm.

Results are shown in FIG. 15.

TABLE V
Abbreviations and Structures

| Abbreviation | Structure |
|---|---|
| Adoa | 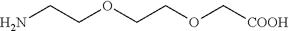 |
| Btn | 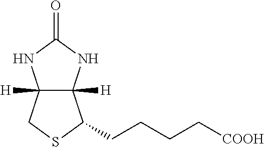 |
| EDA | H₂N—CH₂—CH₂—NH₂ |

TABLE VI
Peptide Sequence, Resin Loading and Yield

| | | Resin used, Loading, mmol/g, g, | Yield in mg |
|---|---|---|---|
| CPD# | Sequence | mmol | (%) |
| 1 | Ac-TMGFTAPRFPHY-NH₂ | Fmoc-PAL-PEG-PS, 0.2 mmol/g, | 167.0 (46%) |
| | | 1.2 g, 0.24 mmol | |
| 2 | Ac-MQSPFTPHFAER-NH₂ | Fmoc-PAL-PEG-PS, 0.2 mmol/g, | 137.0 (38%) |
| | | 1.2 g, 0.24 mmol | |
| 3 | Ac-MQSPIVLPLSLS-NH₂ | Fmoc-PAL-PEG-PS, 0.2 mmol/g, | 131.0 (41%) |
| | | 1.2 g, 0.24 mmol | |
| 4 | Ac-HHEPGAWLPLSP-NH₂ | Fmoc-PAL-PEG-PS, 0.2 mmol/g, | 209.0 (62%) |
| | | 1.2 g, 0.24 mmol | |
| 5 | Btn-Adoa-Adoa- | Fmoc-PAL-PEG-PS, 0.2 mmol/g, | 70.0 (18%) |
| | TMGFTAPRFPHY-NH₂ | 1.0 g, 0.2 mmol | |
| 6 | Btn-Adoa-Adoa-MQSPIVLPLSLS- | Fmoc-PAL-PEG-PS, 0.2 mmol/g, | 140.0 (38%) |
| | NH₂ | 1.0 g, 0.2 mmol | |
| 7 | Btn-Adoa-Adoa- | Fmoc-PAL-PEG-PS, 0.2 mmol/g, | 205.0 (55%) |
| | HHEPGAWLPLSP-NH₂ | 1.0 g, 0.2 mmol | |
| 8 | Btn-Adoa-Adoa- | Fmoc-PAL-PEG-PS, 0.2 mmol/g, | 135.0 (34%) |
| | MQSPFTPHFAER-NH₂ | 1.0 g, 0.2 mmol | |
| 9 | Ac-TMGFTAPRFPHY-DAE-Adoa- | 1,2-Diaminoethane trityl resin, | 65.0 (16%) |
| | Adoa-Btn | 0.9 mmol/g, 0.222 g, 0.2 mmol | |
| 10 | Ac-MQSPFTPHFAER-DAE-Adoa- | 1,2-Diaminoethane trityl resin, | 30.0 (7%) |
| | Adoa-Btn | 0.9 mmol/g, 0.222 g, 0.2 mmol | |
| 11 | Ac-MQSPIVLPLSLS-DAE-Adoa- | 1,2-Diaminoethane trityl resin, | 60.0 (15.7%) |
| | Adoa-Btn | 0.9 mmol/g, 0.222 g, 0.2 mmol | |
| 12 | Ac-HHEPGAWLPLSP-DAE-Adoa- | 1,2-Diaminoethane trityl resin, | 25.0 (7%) |
| | Adoa-Btn | 0.9 mmol/g, 0.222 g, 0.2 mmol | |

TABLE VII
HPLC and Mass Spectral Analysis of Peptides

| Cpd # | RT, Column & Conditions | MS |
|---|---|---|
| 1 | Ret. time: 7.38 min; Assay: >95% (area %); Column: Waters X- | [M + H]: 1465.6, |
| | Terra MS C-18 RP, 50.0 mm × 4.6 mm i.d.; Particle size: 5.0 | [M + 2H]/2: 733.4 |
| | microns; Eluents: A: Water (0.1% TFA), B: acetonitrile (0.1% | |
| | TFA); Elution: Initial condition: 10.0% B, linear gradient 10-40% B | |
| | over 15.0 min; Flow rate: 3.0 mL/min; Detection: UV @ 220 nm. | |
| 2 | Ret. time: 6.48 min; Assay: >95% (area %); Column: Waters X- | [M + H]: 1489.6; |
| | Terra MS-C18 RP, 50.0 mm × 4.6 mm i.d.; Particle size: 5.0 | [M + Na + H]: |
| | microns; Eluents: A: Water (0.1% TFA), B: acetonitrile (0.1% | 755.0; [M + 2H]/2: |
| | TFA); Elution: Initial condition: 10.0% B, linear gradient 10-40% B | 744.8 |
| | over 15.0 min; Flow rate: 3.0 mL/min; Detection: UV @ 220 nm. | |
| 3 | Ret. time: 10.14 min; Assay: >95% (area %); Column: Waters X- | [M + K]: 1364.6; |
| | Terra MS-C18 RP, 50.0 mm × 4.6 mm i.d.; Particle size: 5.0 | [M + Na]: 1348.6 |
| | microns; Eluents: A: Water (0.1% TFA), B: acetonitrile (0.1% | |
| | TFA); Elution: Initial condition: 10.0% B, linear gradient 10-40% B | |
| | over 15.0 min; Flow rate: 3.0 mL/min; Detection: UV @ 220 nm. | |
| 4 | Ret. time: 6.86 min; Assay: >95% (area %); Column: Waters X- | [M + H]: 1382.6; |
| | Terra MS C-18 RP, 50.0 mm × 4.6 mm i.d.; Particle size: 5.0 | [M + Na]: 1403.6 |
| | microns; Eluents: A: Water (0.1% TFA), B: acetonitrile (0.1% | |
| | TFA); Elution: Initial condition: 10.0% B, linear gradient 10-40% B | |
| | over 15.0 min; Flow rate: 3.0 mL/min; Detection: UV @ 220 nm. | |
| 5 | Ret. time: 5.63 min; Assay: >95% (area %); Column: Waters | [M + H]: 1940.6; |
| | Sunfire ™ C-8 RP, 50.0 mm × 4.6 mm i.d.; Particle size: 3.5 | [M + 2H]/2: 970.8 |
| | microns; Eluents: A: Water (0.1% TFA), B: acetonitrile (0.1% | |
| | TFA); Elution: Initial condition: 15.0% B, linear gradient 15-45% B | |
| | over 15.0 min; Flow rate: 3.0 mL/min; Detection: UV @ 220 nm. | |
| 6 | Ret. time: 8.53 min; Assay: >95% (area %); Column: Waters | [M + Na]: 1823.8; |
| | Sunfire ™ C-8 RP, 50.0 mm × 4.6 mm i.d.; Particle size: 3.5 | [M + H]: 18 00.8; |
| | microns; Eluents: A: Water (0.1% TFA), B: acetonitrile (0.1% | [M + 2Na]/2; |
| | TFA); Elution: Initial condition: 15.0% B, linear gradient 15-45% B | 922.5; [M + 2H]/2: |
| | over 15.0 min; Flow rate: 3.0 mL/min; Detection: UV @ 220 nm | 900.5 |
| 7 | Ret. time: 5.57 min; Assay: >95% (area %); Column: Waters | [M + H]: 18 55.5; |
| | Sunfire ™ C-8 RP, 50.0 mm × 4.6 mm i.d.; Particle size: 3.5 | [M + 2H]/2: 928.5 |
| | microns; Eluents: A: Water (0.1% TFA), B: acetonitrile (0.1% | |
| | TFA); Elution: Initial condition: 15.0% B, linear gradient 15-45% B | |
| | over 15.0 min; Flow rate: 3.0 mL/min; Detection: UV @ 220 nm | |
| 8 | Ret. time: 5.29 min; Assay: >95% (area %); Column: Waters | [M + H]: 1964.5; |
| | Sunfire ™ C-8 RP, 50.0 mm × 4.6 mm i.d.; Particle size: 3.5 | [M + 2H]/2: 982.0 |
| | microns; Eluents: A: Water (0.1% TFA), B: acetonitrile (0.1% | |
| | TFA); Elution: Initial condition: 15.0% B, linear gradient 15-45% B | |
| | over 15.0 min; Flow rate: 3.0 mL/min; Detection: UV @ 220 nm | |
| 9 | Ret. time: 5.73 min; Assay: >95% (area %); Column: Waters | [M + H]: 2025.5; |
| | Sunfire ™ C-8 RP, 50.0 mm × 4.6 mm i.d.; Particle size: 3.5 | [M + 2H]/2: |
| | microns; Eluents: A: Water (0.1% TFA), B: acetonitrile (0.1% | 1013.3 |
| | TFA); Elution: Initial condition: 15.0% B, linear gradient 15-45% B | |
| | over 15.0 min; Flow rate: 3.0 mL/min; Detection: UV @ 220 nm | |
| 10 | Ret. time: 5.29 min; Assay: >90% (area %); Column: Waters | [M + H]: 2047.8; |
| | Sunfire ™ C-8 RP, 50.0 mm × 4.6 mm i.d.; Particle size: 3.5 | [M + 2H]/2: |
| | microns; Eluents: A: Water (0.1% TFA), B: acetonitrile (0.1% | 1024.7 |
| | TFA); Elution: Initial condition: 15.0% B, linear gradient 15-45% B | |
| | over 15.0 min; Flow rate: 3.0 mL/min; Detection: UV @ 220 nm | |
| 11 | Ret. time: 8.41 min; Assay: >90% (area %); Column: Waters | [M + H]: 1906.8; |
| | Sunfire ™ C-8 RP, 50.0 mm × 4.6 mm i.d.; Particle size: 3.5 | [M + 2H]/2: 965.0 |
| | microns; Eluents: A: Water (0.1% TFA), B: acetonitrile (0.1% | |
| | TFA); Elution: Initial condition: 15.0% B, linear gradient 15-45% B | |
| | over 15.0 min; Flow rate: 3.0 mL/min; Detection: UV @ 220 nm | |
| 12 | Ret. time: 5.73 min; Assay: >95% (area %); Column: Waters | [M + H]: 1940.8; |
| | Sunfire ™ C-8 RP, 50.0 mm × 4.6 mm i.d.; Particle size: 3.5 | [M + 2H]/2: 971.0 |
| | microns; Eluents: A: Water (0.1% TFA), B: acetonitrile (0.1% | |
| | TFA); Elution: Initial condition: 15.0% B, linear gradient 15-45% B | |
| | over 15.0 min; Flow rate: 3.0 mL/min; Detection: UV @ 220 nm | |

TABLE VIII
Structures of Peptides

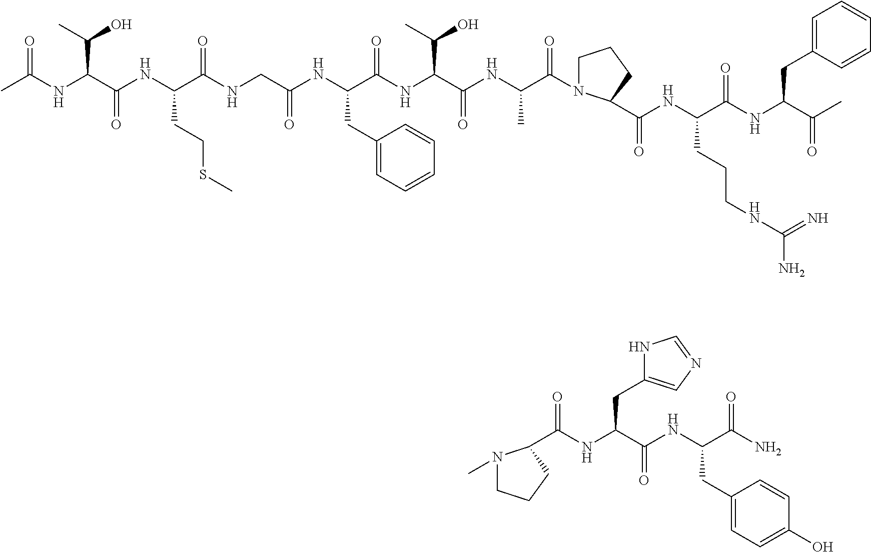

Compound 1
Ac-TMGFTAPRFPHY-NH₂

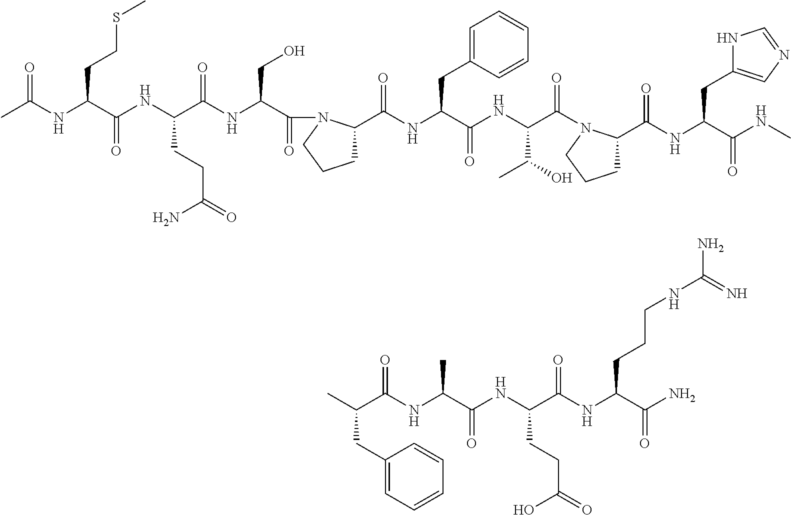

Compound 2
Ac-MQSPFTPHFAER-NH₂

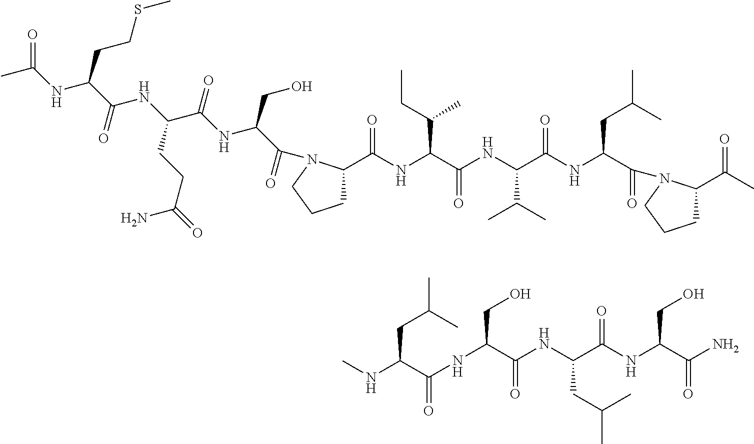

Compound 3
Ac-MQSPIVLPLSLS-NH₂

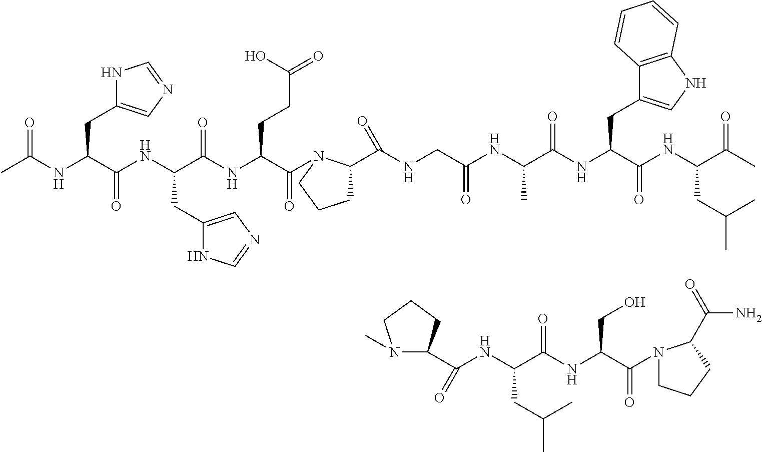

Compound 4
Ac-HHEPGAWLPLSP-NH₂

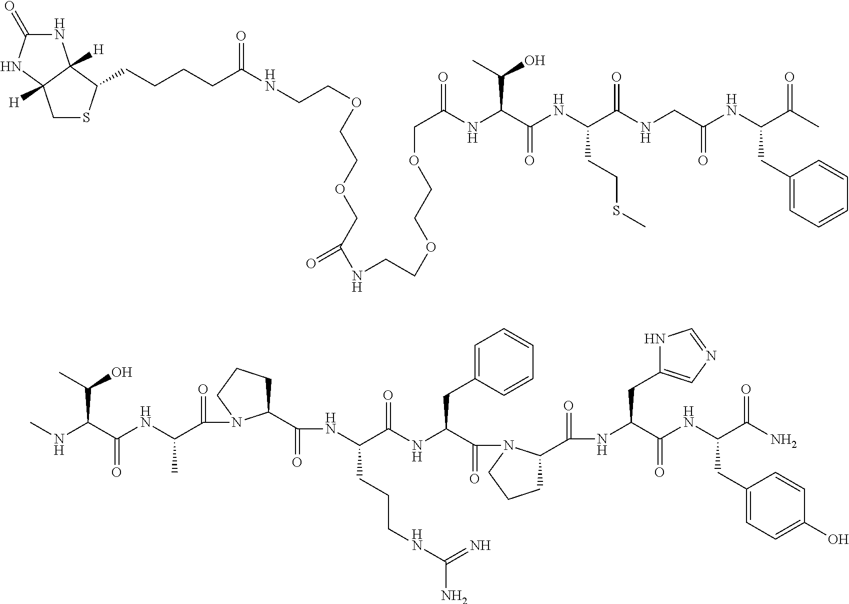

Compound 5
Btn-Adoa-Adoa-TMGFTAPRFPHY-NH₂

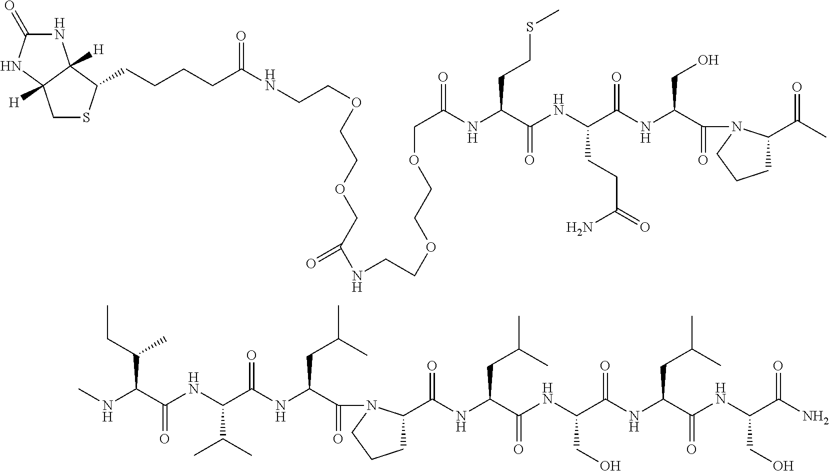

Compound 6
Btn-Adoa-Adoa-MQSPIVLPLSLS-NH₂

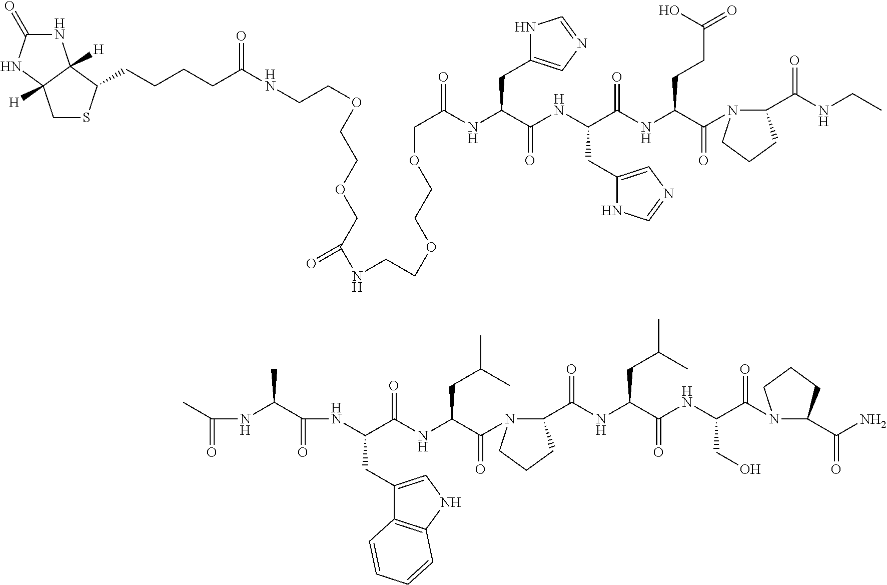

Compound 7
Btn-Adoa-Adoa-HHEPGAWLPLSP-NH₂

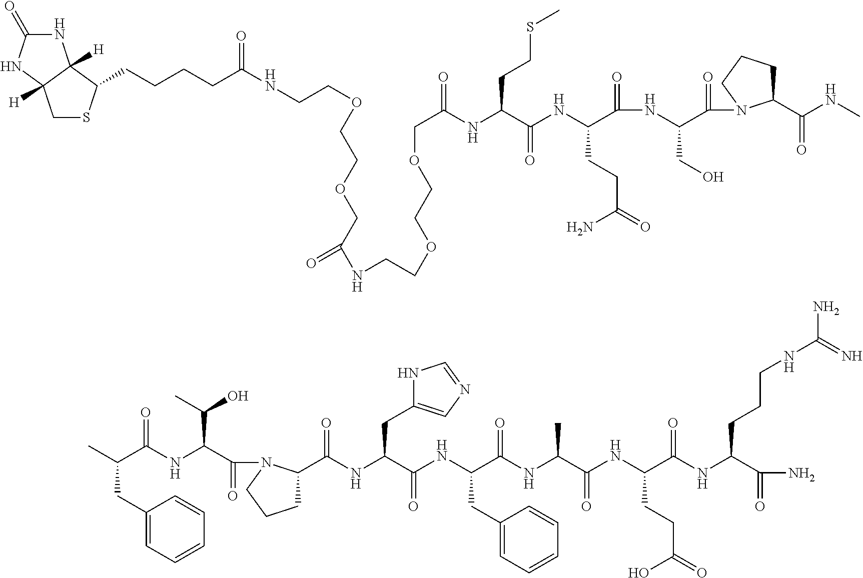

Compound 8
Btn-Adoa-Adoa-MQSPFTPHFAER-NH₂

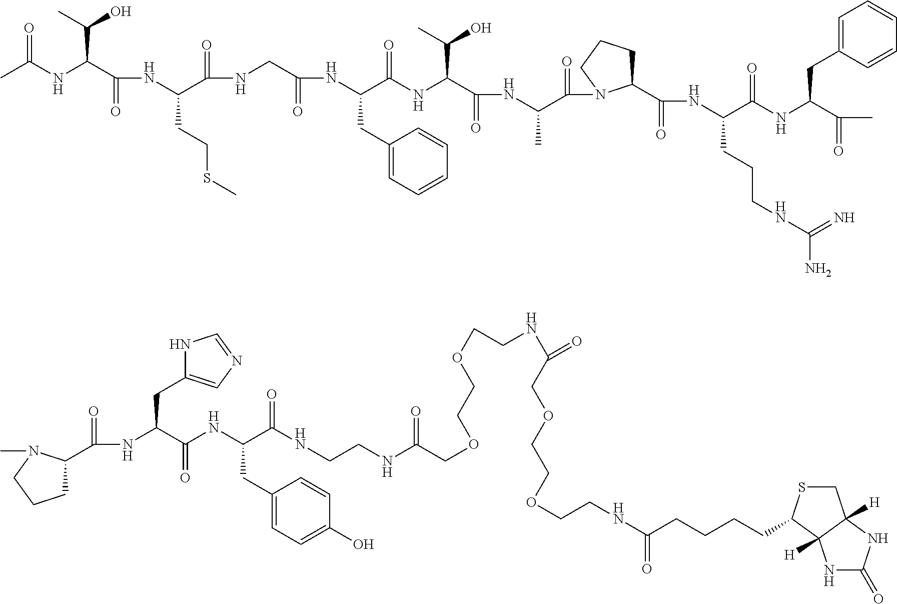

Compound 9
Ac-TMGFTAPRFPHY-DAE-Adoa-Adoa-Btn

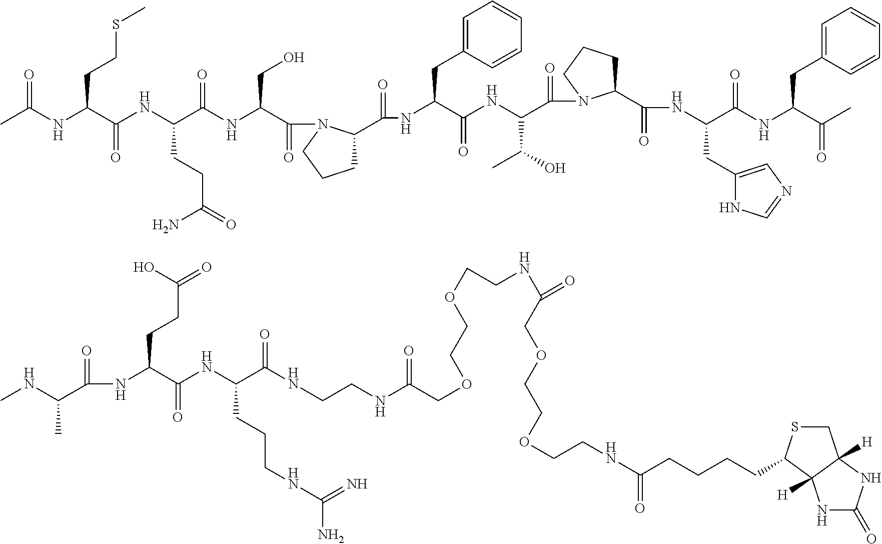

Compound 10
Ac-MQSPFTPHFAER-DAE-Adoa-Adoa-Btn

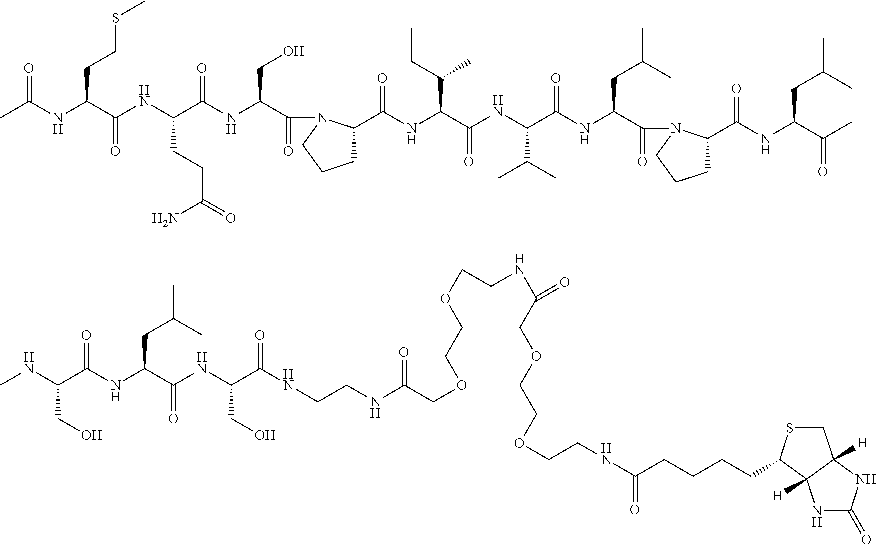

Compound 11
Ac-MQSPIVLPLSLS-DAE-Adoa-Adoa-Btn

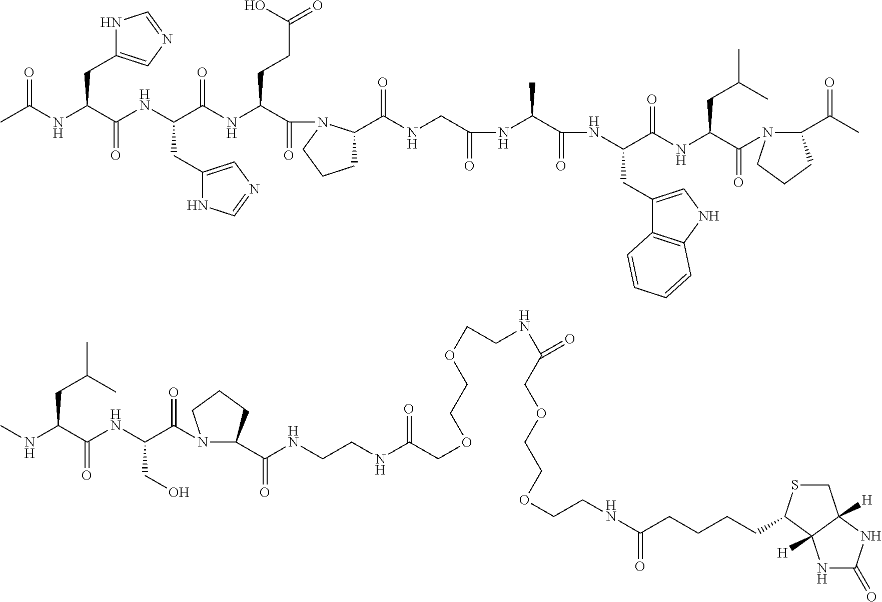

Compound 12
Ac-HHEPGAWLPLSP-DAE-Adoa-Adoa-Btn

### Embodiments

A list of certain, non-limiting embodiments of the invention follows:
1. An isolated polynucleotide molecule comprising any one of the odd-numbered Sequence ID from 1 to 389 and from 395 to 453 or any fragment thereof and coding for an amino acidic sequence comprising any one of the even-numbered Sequence ID from 2 to 390 and from 396 to 454 or any fragment thereof.
2. An amino acid sequence comprising any one of the even-numbered Sequence ID from 2 to 390 and from 396 to 454 or any homologous sequence or any sequence bearing conservative substitutions, which binds to the antigen possibly found in the coronary plaque or any fragment thereof.
3. The isolated amino acid sequences according to embodiment 2 and corresponding to Sequence ID No 22, 38, 44, 52 and 54.
4. An amino acid sequence of embodiment 2 having a germline homology of at least 80%, preferably of at least 90%, more preferably of at least 95% and even more preferably of at least of 97%; or any fragment thereof.
5. An amino acid sequence of embodiment 2 or 3 having a p-value of the CDR3 portion less than 5%, preferably less than 2% , more preferably less than 1% and even more preferably less than 1%, or any fragment thereof.
6. An amino acid sequence of embodiments 2 to 5 encoded by a polynucleotide molecule of embodiment 1 or any fragments thereof.
7. An expression vector comprising one or more of the isolated polynucleotide molecules of embodiment 1.
8. The expression vector of embodiment 7 comprising Sequence ID No 53 and, optionally, any one of the sequences set forth in Sequence ID Nos 21, 37, 43 and 51.
9. The expression vector of embodiment 7 or 8 selected from the group comprising plasmids, cosmids, YACs, viral particles or phages.
10. An expression system comprising one or more expression vector according to embodiment 7.
11. An isolated recombinant host cell comprising the expression system of embodiment 10.
12. The isolated recombinant host cell of embodiment 11 selected from the group comprising prokaryotic recombinant isolated cells such as Enterobacter, Escherichia, Erwinia, Klebsiella, Proteus, Salmonella, Serratia, Shigella, Bacilli, Pseudomonas and Strepromyces; preferably said prokaryotic recombinant isolated cell is selected from the group comprising E. coli, Salmonella typhimurium, Serratia marcescans, Bacillus subtilis, Bacillus licheniformis, Pseudomonas aeruginosa and even more preferably said prokaryotic recombinant isolated host cell is E. coli XL1 -Blue; yeast recombinant host cells such as Saccharomyces, Pichia pastoris, Kluyveromyces such as K. lactis, K. fragilis, K. bulgaricus, K. wickeramii, K. waltii, K. drosophilarum, K. thermotolerans, K. marxianus, Schizosaccharomyces, such as Schizosaccharomyces pombe, yarrowia, Hansenula, Trichoderma reesia, Neurospora crassa, Schwanniomyces such as Schwanniomyces occidentalis, Neurospora, Penicillium, Tolypociadium, Aspergillus such as A. nidulans, Candida, Torulopsis and Rhodotorula; preferably said yeast recombinant host cell being Saccharomyces cerevisiae; human recombinant isolated host cell such as Chinese hamster ovary (CHO), monkey kidney CVI line transformed by SV40 (COS-7, ATCC CRL 1651), human embryonic kidney line, Chinese hamster ovary cells/-DHFR, mouse sertoli cells, human lung cells (W138, ATCC CCL 75); human liver cells (Hep G2, HB 8065); and mouse mammary tumor (MMT 060562, ATCC CCL51); plant isolated recombinant host cells such as Agrobacterium tumefaciens and Nicotiana tabacum; insect recombinant isolated cells such as Drosophila S2 and Spodoptera Sf9.
13. A process for the preparation of recombinant antibodies or of any fragments thereof including the steps of:
   a) preparing an expression system comprising an expression vector comprising one or more polynucleotidic molecules corresponding to any one of the polynucleotidic sequences of embodiment 1 and a host cell comprising said expression vector;
   b) culturing said host cell under suitable growth conditions;
   c) recovering the antibodies or any fragments thereof thus produced; and
   d) purifying said antibodies or any fragments thereof.
14. The process of embodiment 13 wherein the one or more polynucleotide molecules of step a) is or are selected from the odd-numbered sequences of Sequence ID from 1 to 389 and from 395 to 453.
15. The process of embodiments 13 or 14 wherein the recombinant isolated host cell is selected from the group comprising E. coli, B. subtilis, S. Cerevisiae or Chinese hamster ovary (CHO).
16. The process according to any one of embodiments 13 to 15 for the preparation of IgG antibodies or any fragment thereof.
17. The process according to any one of embodiments 13 to 15 for the preparation of IgG antibodies Fab fragments.
18. Recombinant isolated antibody or any fragment thereof produced according to the process of embodiment 13 to 15.
19. The recombinant IgG antibodies or any fragment thereof produced according to the process of embodiments 13 to 15.
20. Recombinant IgG Fab fragments produced according to the process of embodiments 13 to 15.
21. Recombinant isolated IgG Fab fragments produced according to the process of embodiments 13 to 16 and comprising any one of the amino acid sequences set forth in Sequence ID Nos 22, 38, 44, 52 and 54.
22. The recombinant isolated IgG Fab fragments of embodiment 21 further produced according to the process of embodiments 13 to 16.
23. The recombinant isolated IgG Fab fragments of embodiment 21 which bind to the antigen possibly present in the coronary plaque.
24. A therapeutic composition comprising a recombinant antibody or any fragment thereof according to any one of embodiments 18 to 23 and, optionally, a therapeutic moiety.
25. The therapeutic composition of embodiment 24 wherein the therapeutic moiety is selected from the group comprising radionuclides, drugs and prodrugs, hormones, hormone antagonists, receptor antagonists, enzymes or proenzymes activated by another agent, autocrines or cytokines, antimicrobial agents and toxins.
26. A diagnostic composition comprising the recombinant antibody or any fragment thereof of any one of embodiments 18 to 23 and a diagnostic moiety.
27. The therapeutic composition of embodiment 24 or 25 for the treatment of the acute coronary syndrome (ACS).
28. The diagnostic composition of embodiment 26 for the diagnosis of the acute coronary syndrome (ACS).
29. A ligand which binds to the amino acidic sequences of any one of embodiments 2 to 6.
30. A ligand which binds to the recombinant antibody or to any fragment thereof of any one of embodiments 18 to 23.
31. A peptide comprising the amino acid consensus sequence and which binds to the recombinant antibody or to a fragment thereof of any one of embodiments 18to 23
32. A peptide of embodiment 31 having an amino acidic corresponding to Sequence ID from 391 to 394.
33. The ligand of embodiment 29 which is selected by a method including the use of the isolated amino acidic sequences of embodiments 2 to 6.
34. The ligand of embodiment 30 which is selected by a method including the use of the recombinant antibody of any one of embodiments 18 to 23.
35. A method for the identification of a ligand which binds to the recombinant antibodies or to any fragment thereof of any one of embodiments of embodiments 18 to 23, said method including the steps of:
   a) binding said antibodies or any fragment thereof onto a solid phase;
   b) removing unbound material by one or more washing steps;
   c) contacting the candidate molecule with the solid phase prepared in step a) and allowing incubation of the candidate molecule and the solid phase for a suitable period of time;
   d) removing unbound material by one or more washing steps;
   e) adding a secondary antibody specific for the complex of the antibody of step a) with the candidate molecule bound thereto; and
   f) identifying the bound molecule to the antibodies of step a).
36. The method of embodiment 35 wherein the antibodies or fragments thereof of step a) are the antibodies or fragments thereof according to embodiments 18 to 23.
37. A method for the identification of a ligand which binds to the amino acidic sequences of any one of embodiments 2 to 6; or to any fragments thereof, said method including the steps of:
   a) binding said amino acidic sequences or any fragment thereof onto a solid phase;
   b) removing unbound material by one or more washing steps;
   c) contacting the candidate molecule with the solid phase prepared in step a) and allowing incubation of the candidate molecule and the solid phase for a suitable period of time;
   d) removing unbound material by one or more washing steps;
   e) adding a secondary antibody specific for the complex of the amino acidic sequence of step a) with the candidate molecule bound thereto; and
   f) identifying the bound molecule to the antibodies of step a).
38. The method of embodiment 36 wherein the amino acid sequences or any fragment thereof of step a) are the amino acid sequences or any fragments thereof of embodiment 2 to 6.
39. An ex-vivo or in vitro diagnostic method comprising the step of contacting a sample selected from the group comprising whole blood, serum and coronary plaque fragment with the antibody or any fragment thereof of any one of embodiments 19 to 23.
40. The ex-vivo or in vitro diagnostic method of embodiment 39 for the diagnosis of acute coronary syndrome (ACS) in a patient.
41. The ex-vivo or in vitro diagnostic method of embodiment 39 for the screening of the population at risk of acute coronary syndrome (ACS).

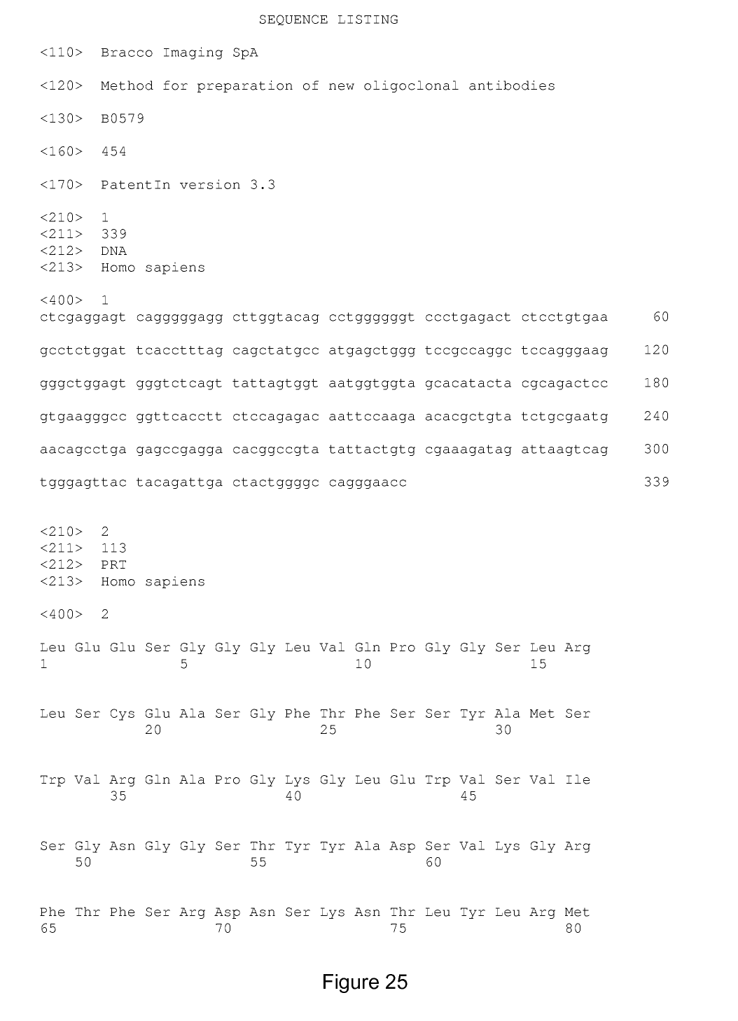

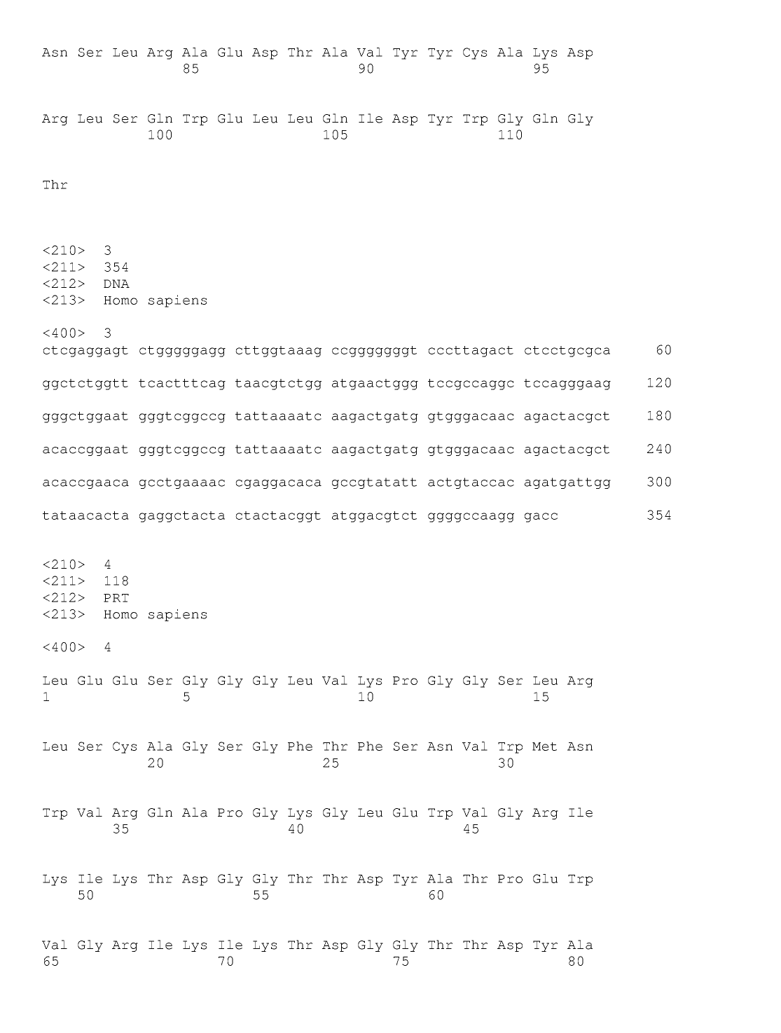

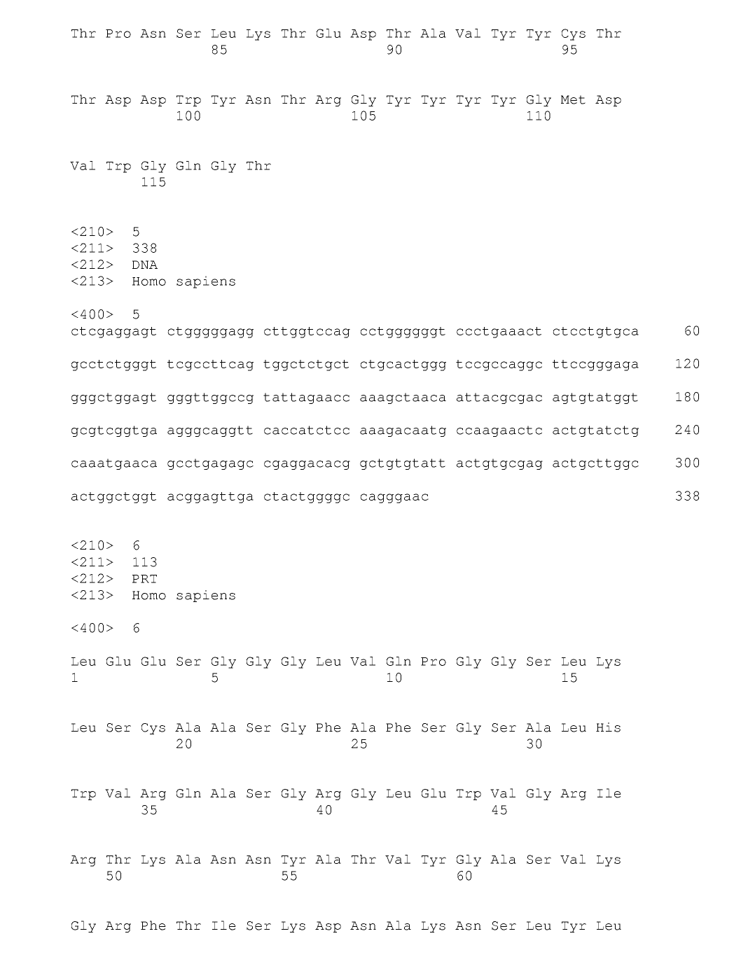

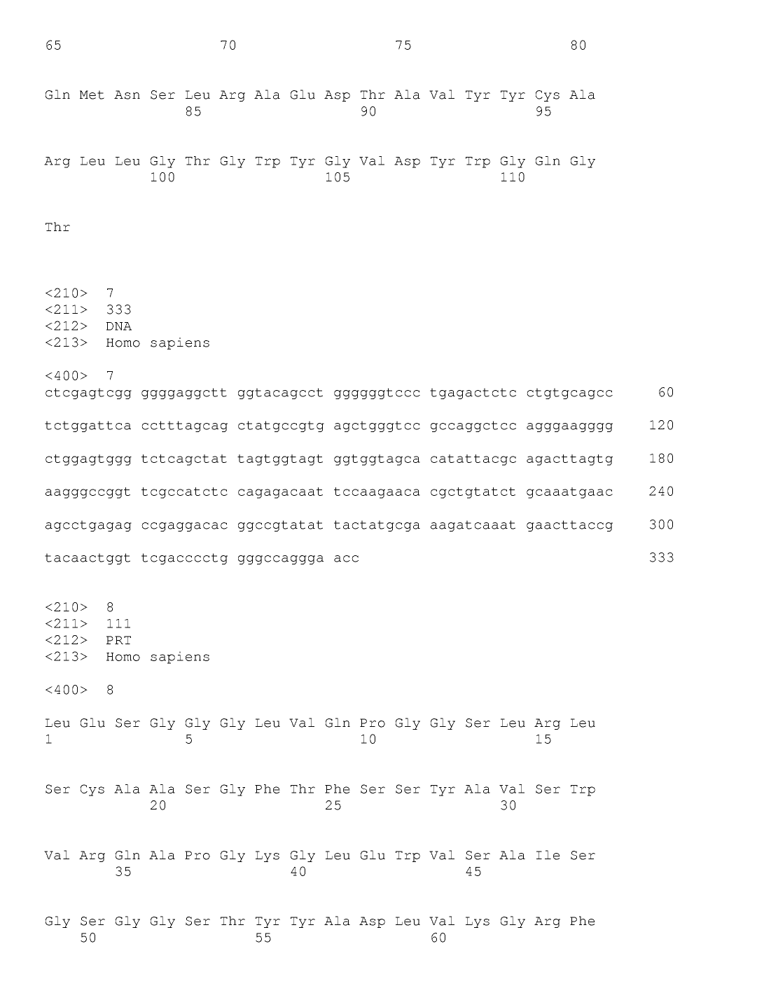

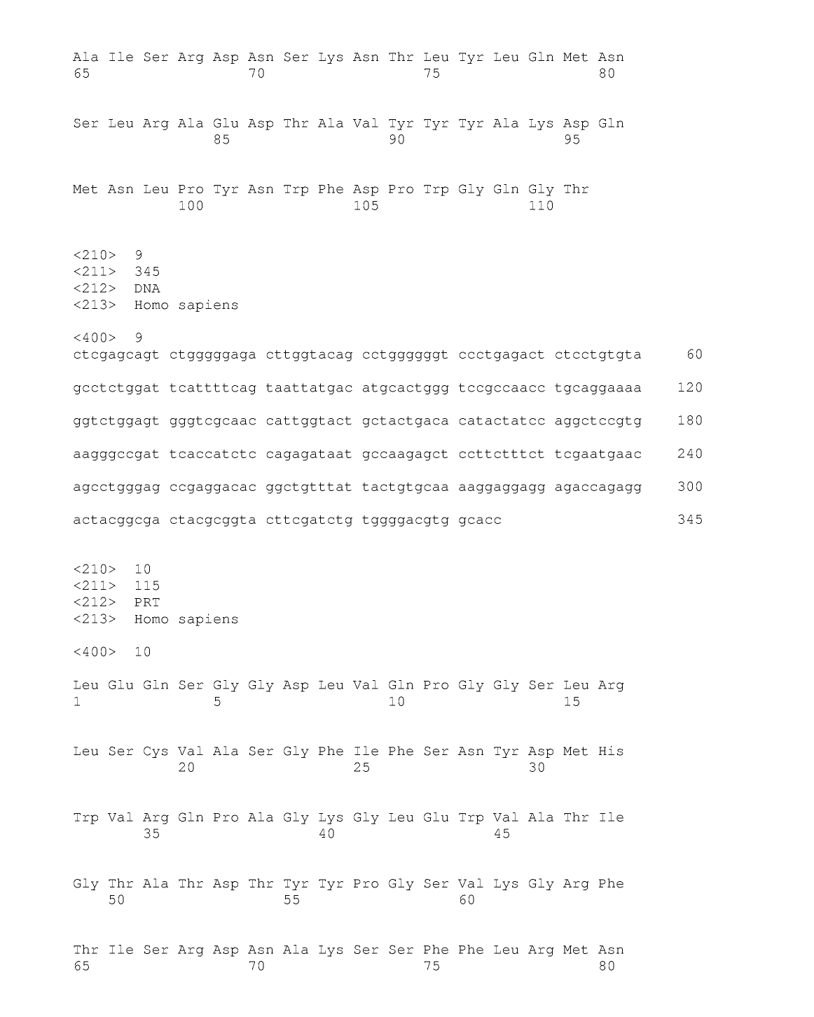

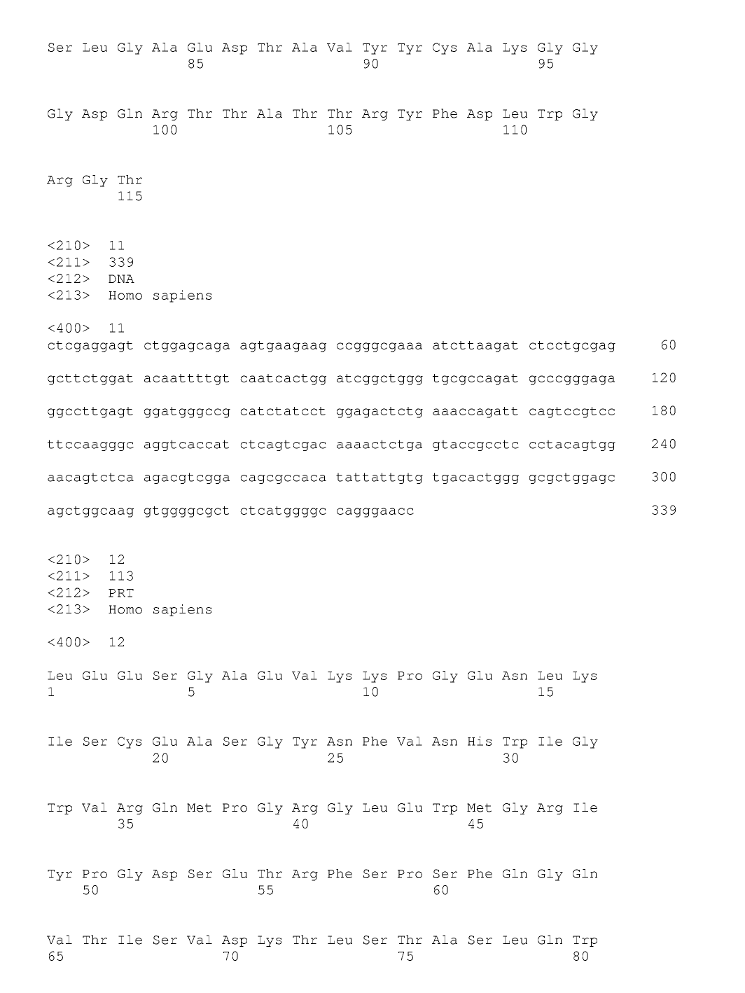

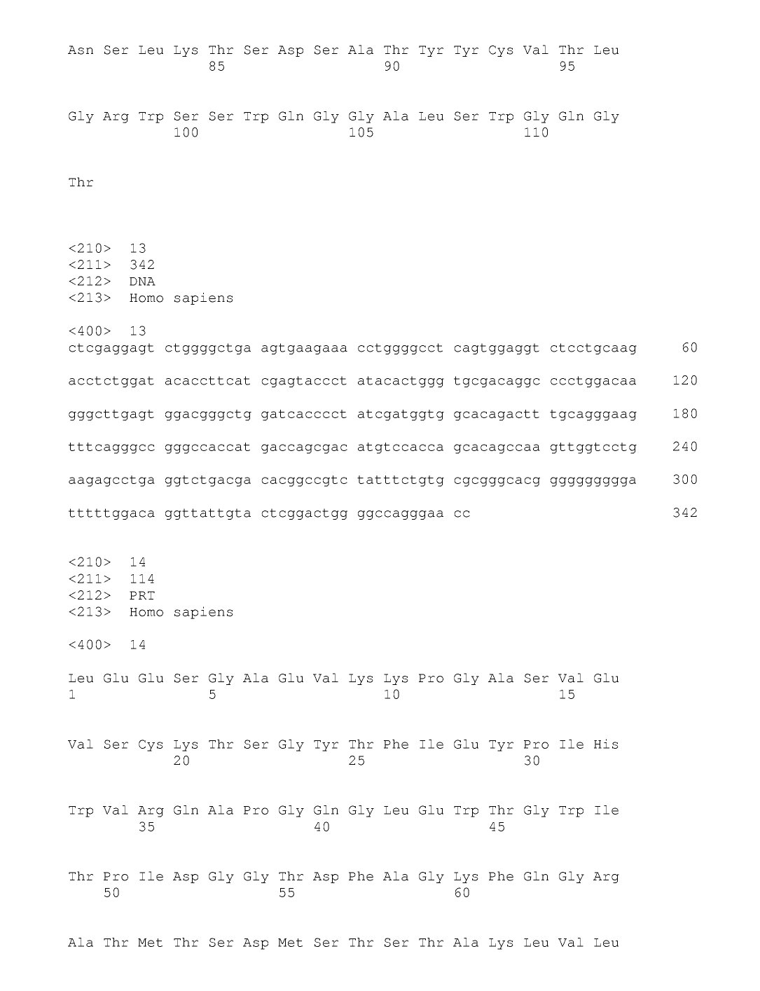

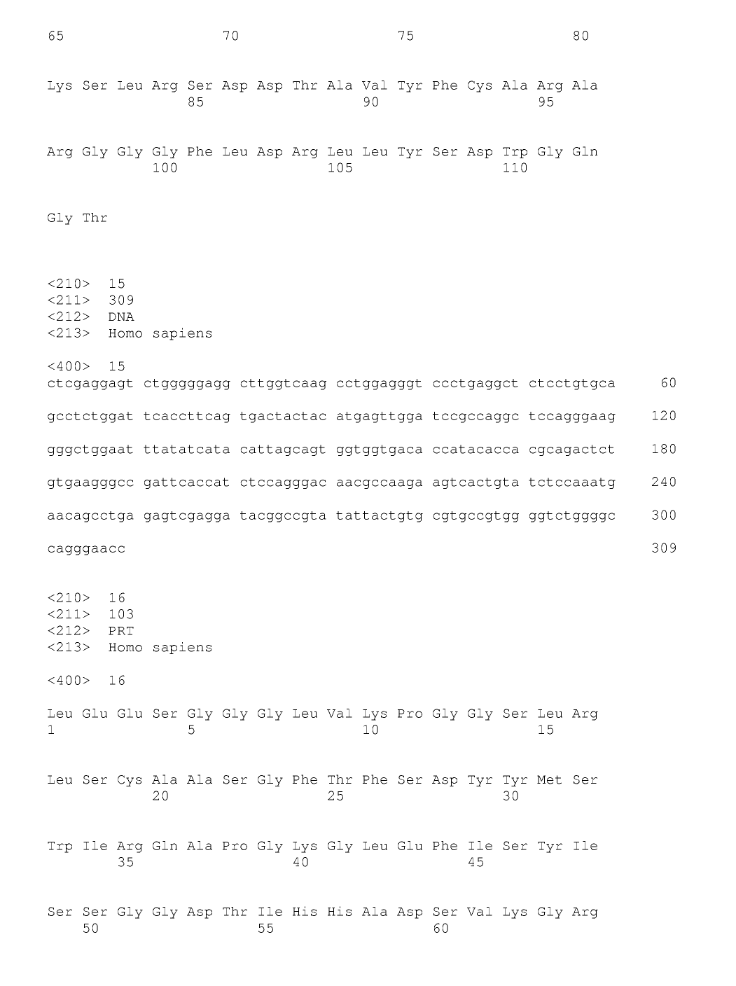

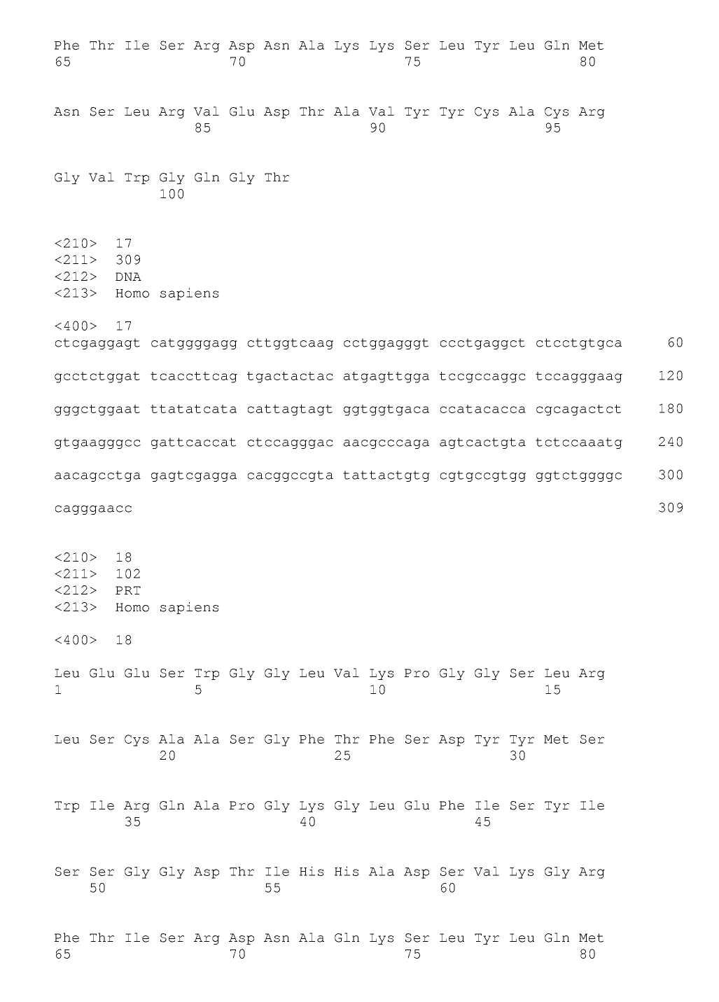

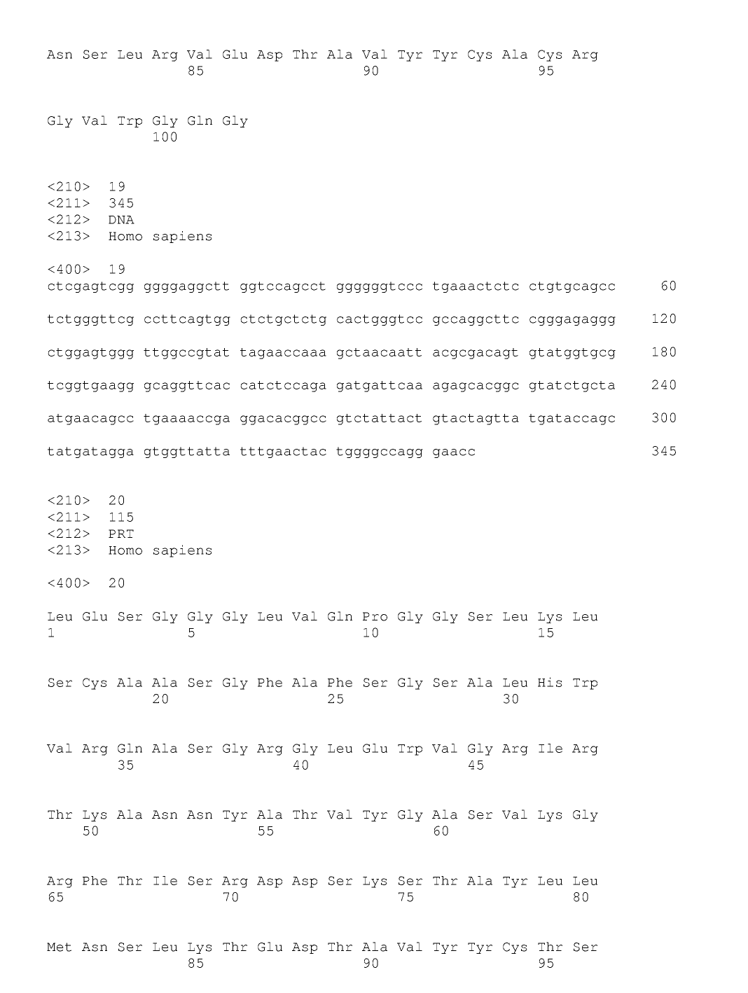

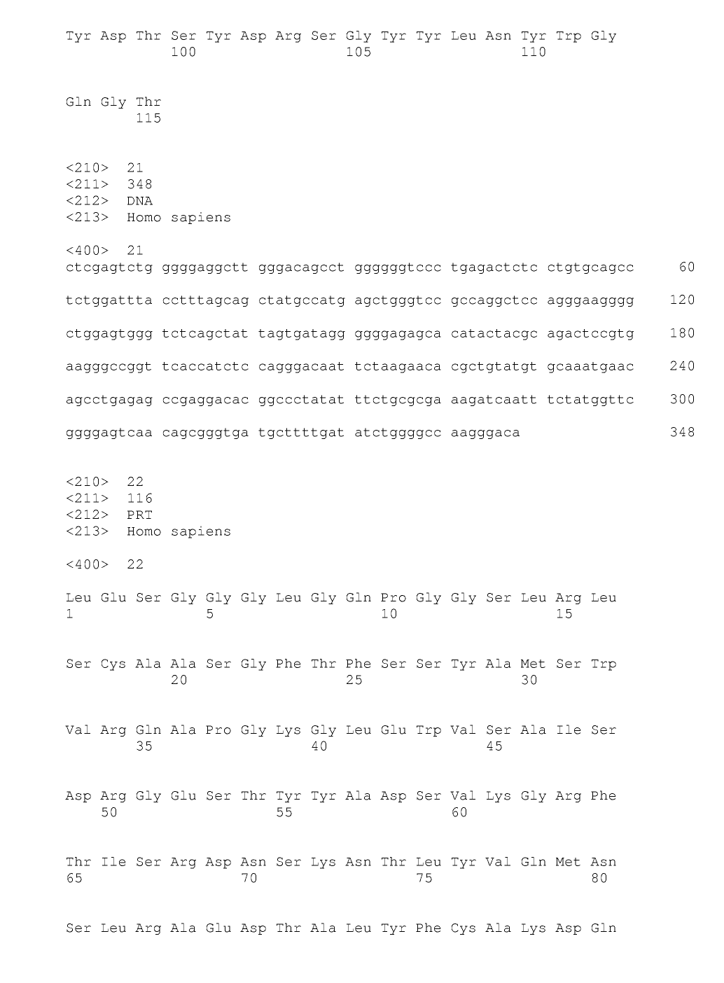

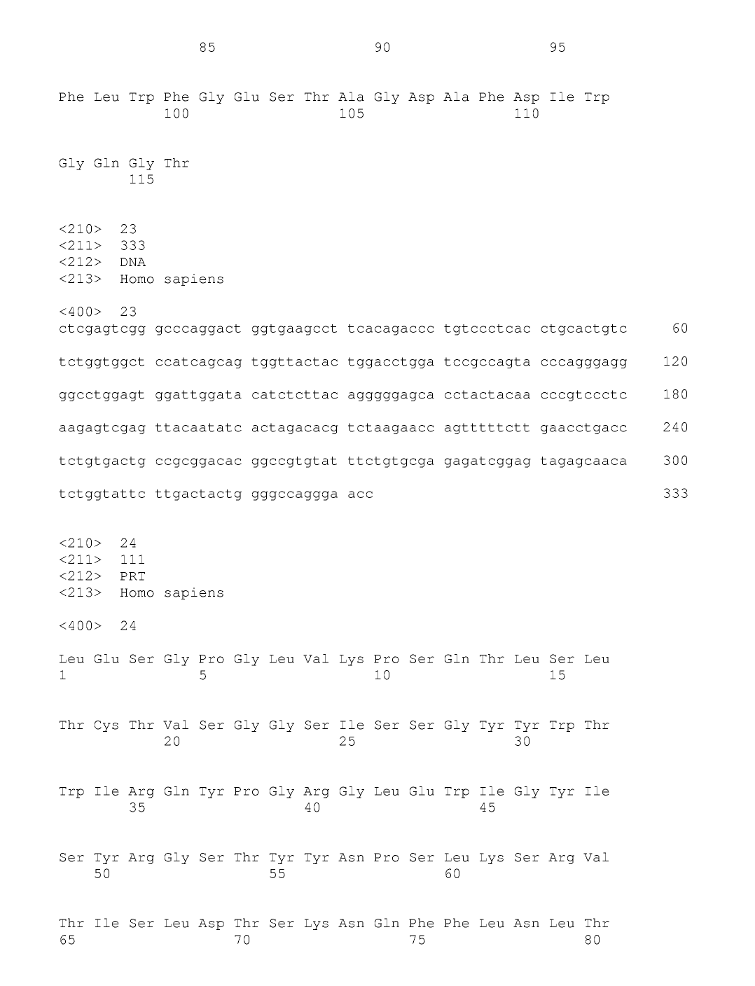

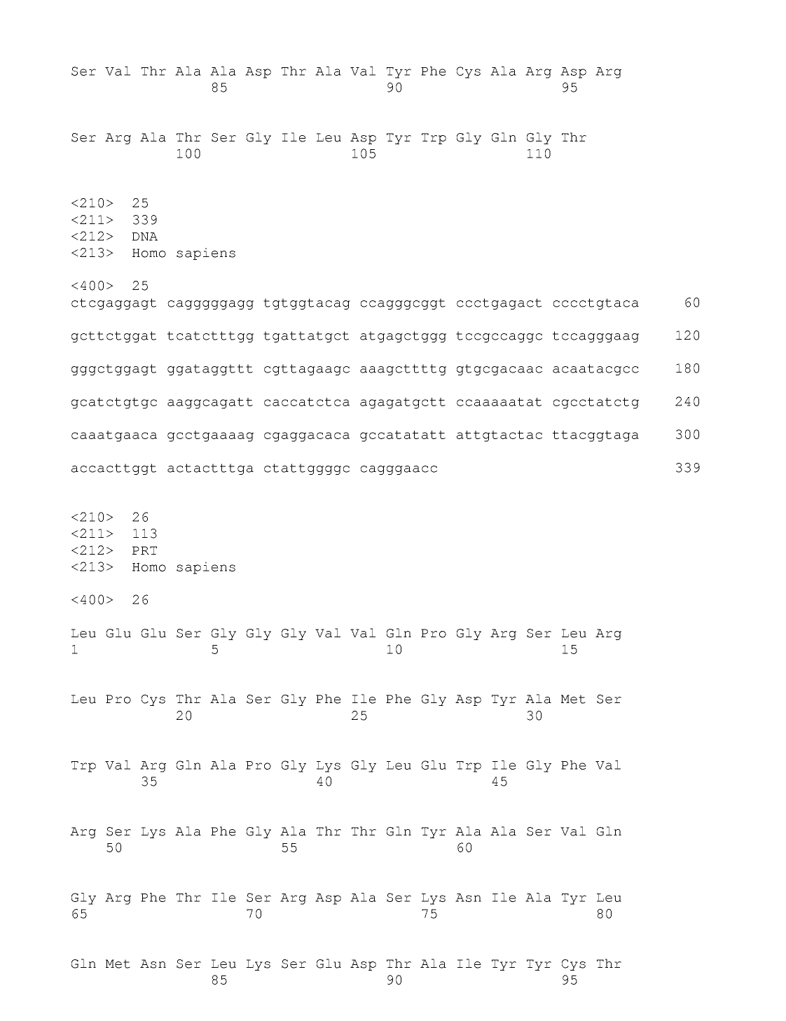

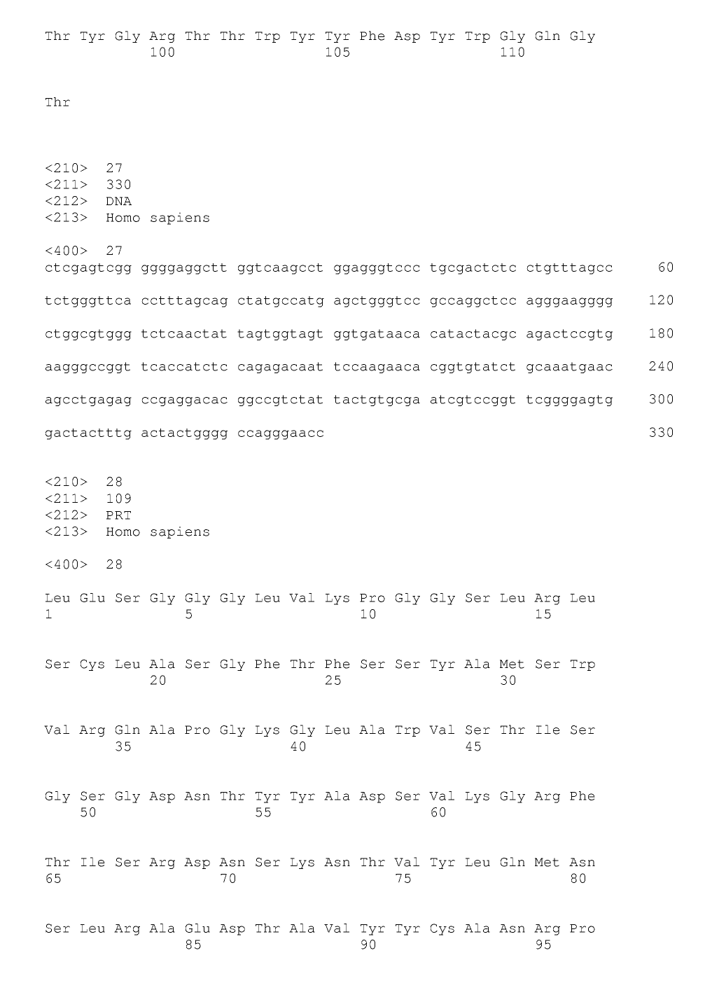

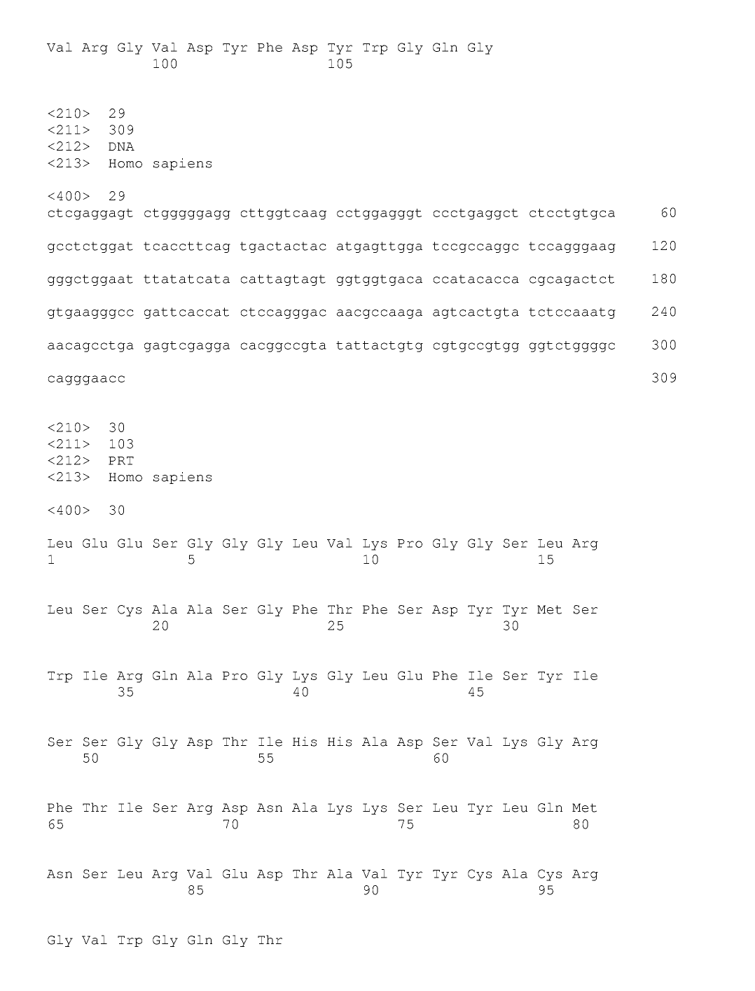

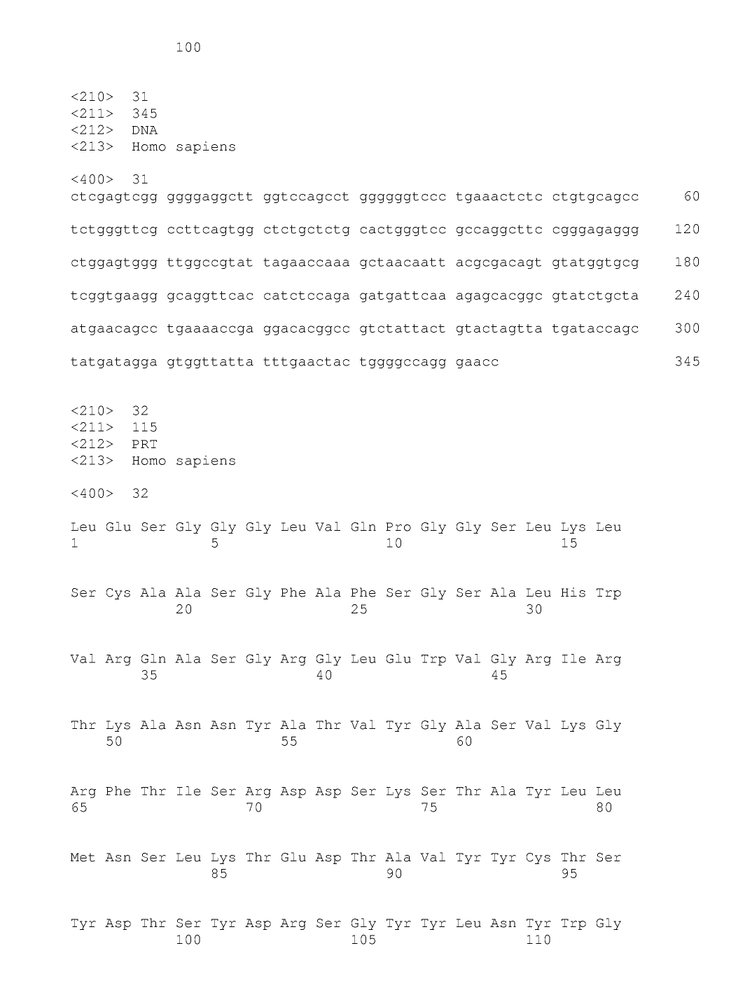

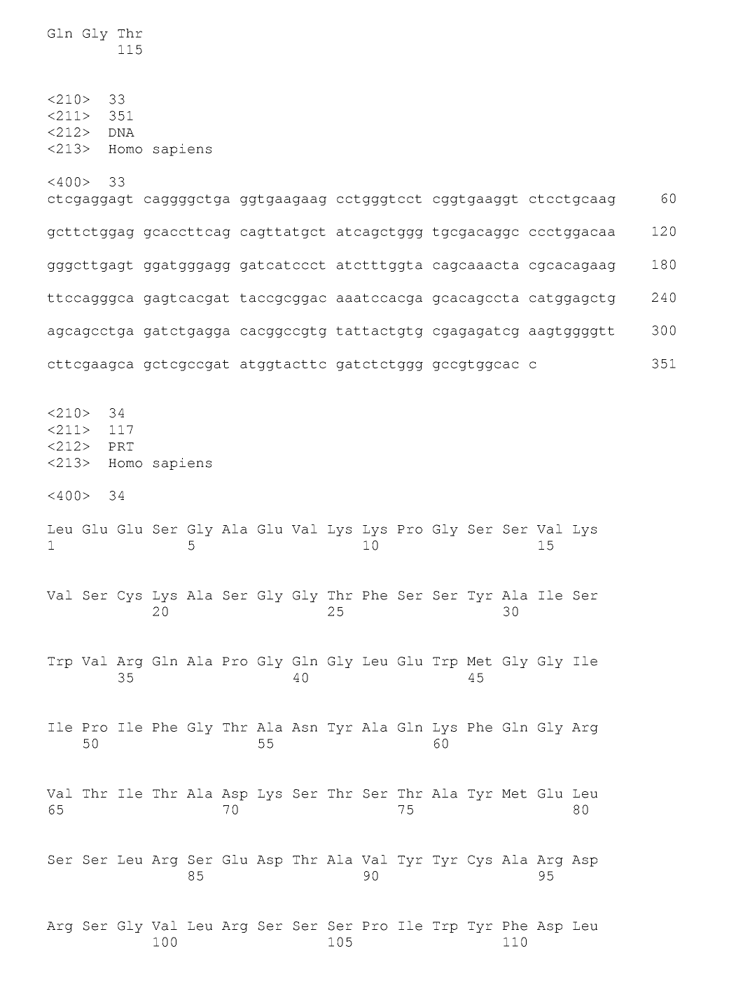

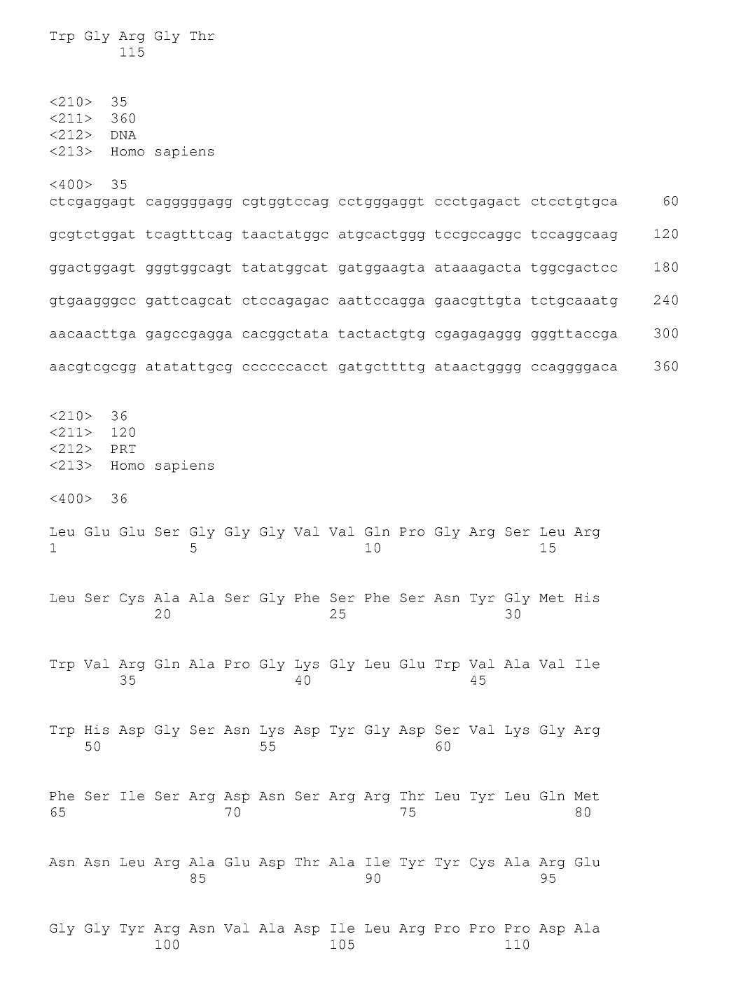

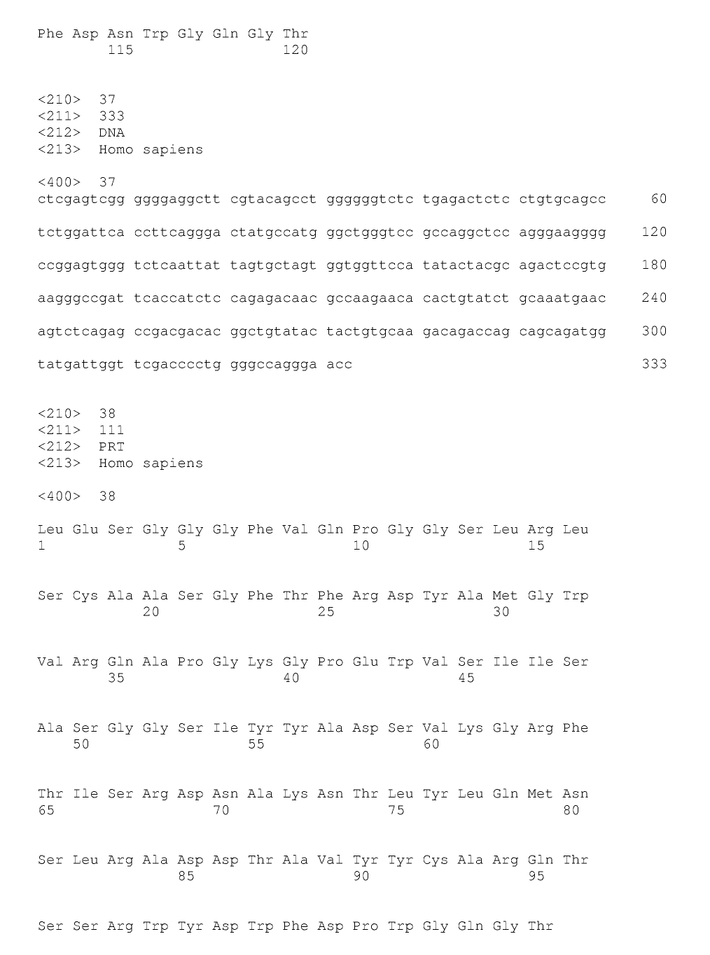

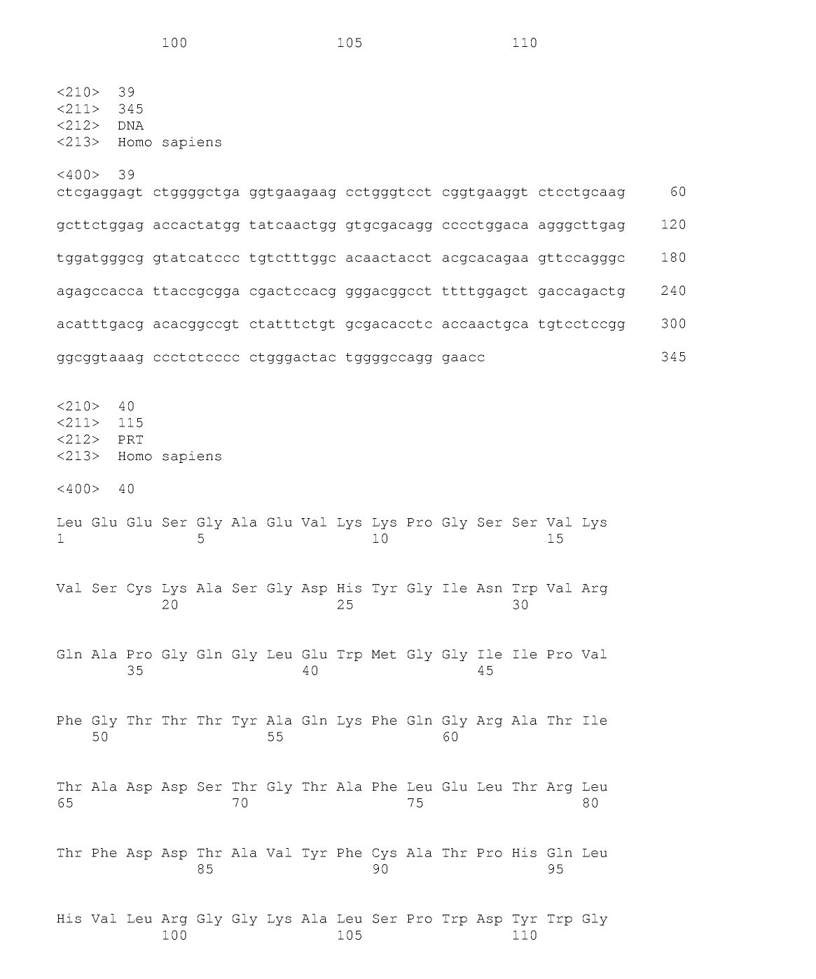

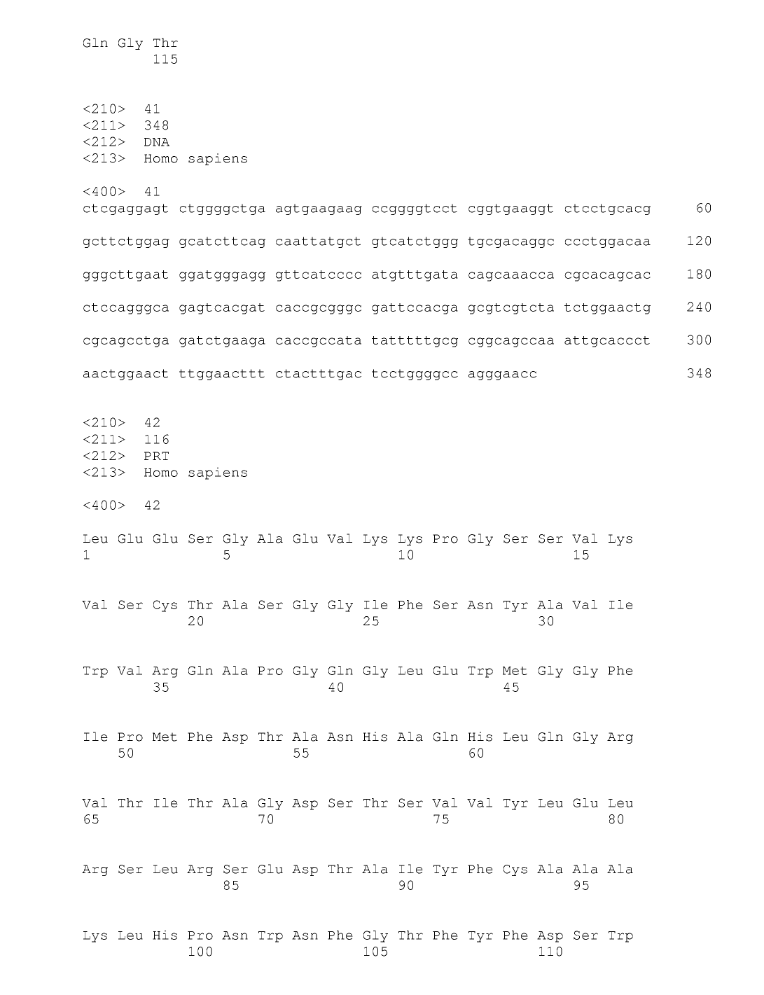

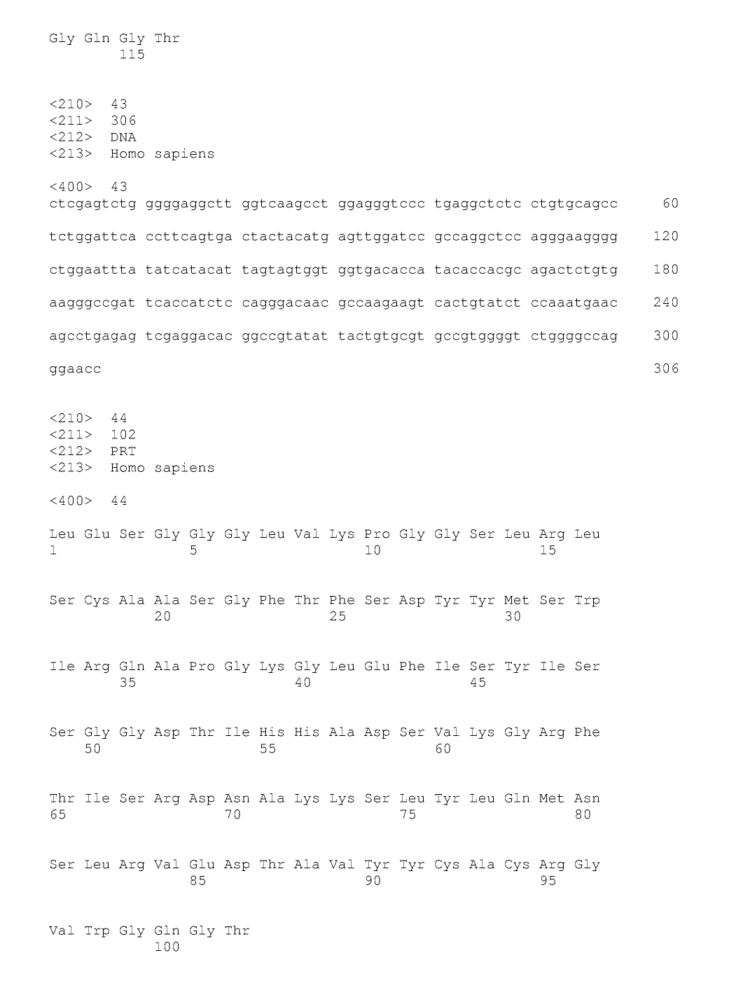

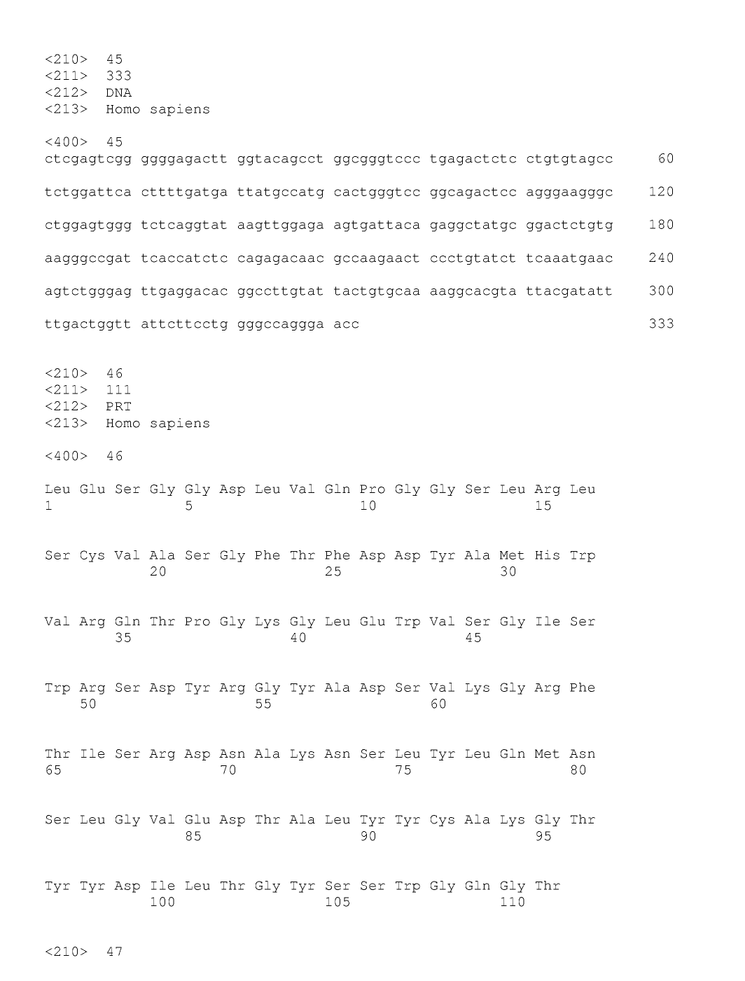

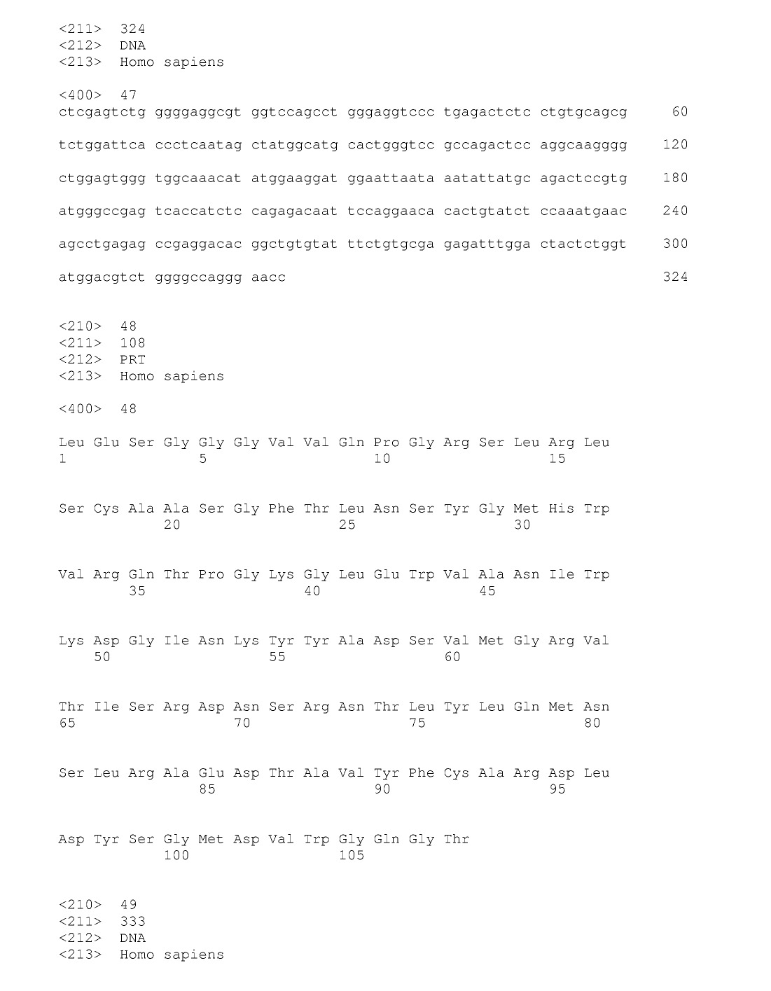

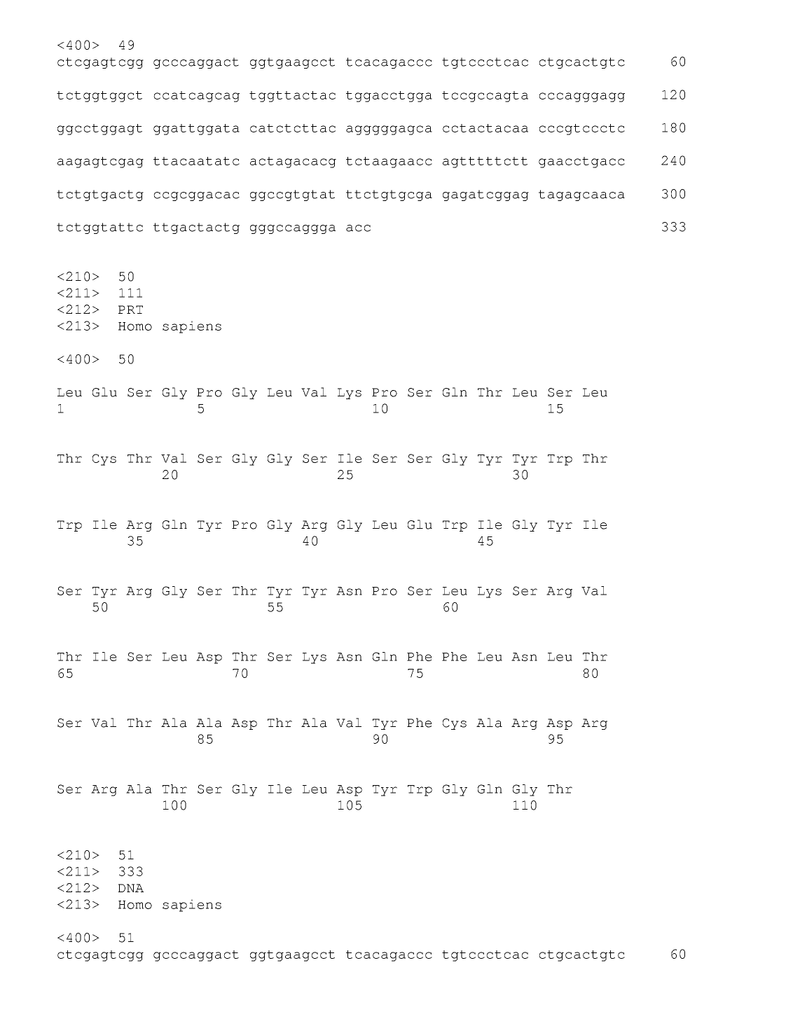

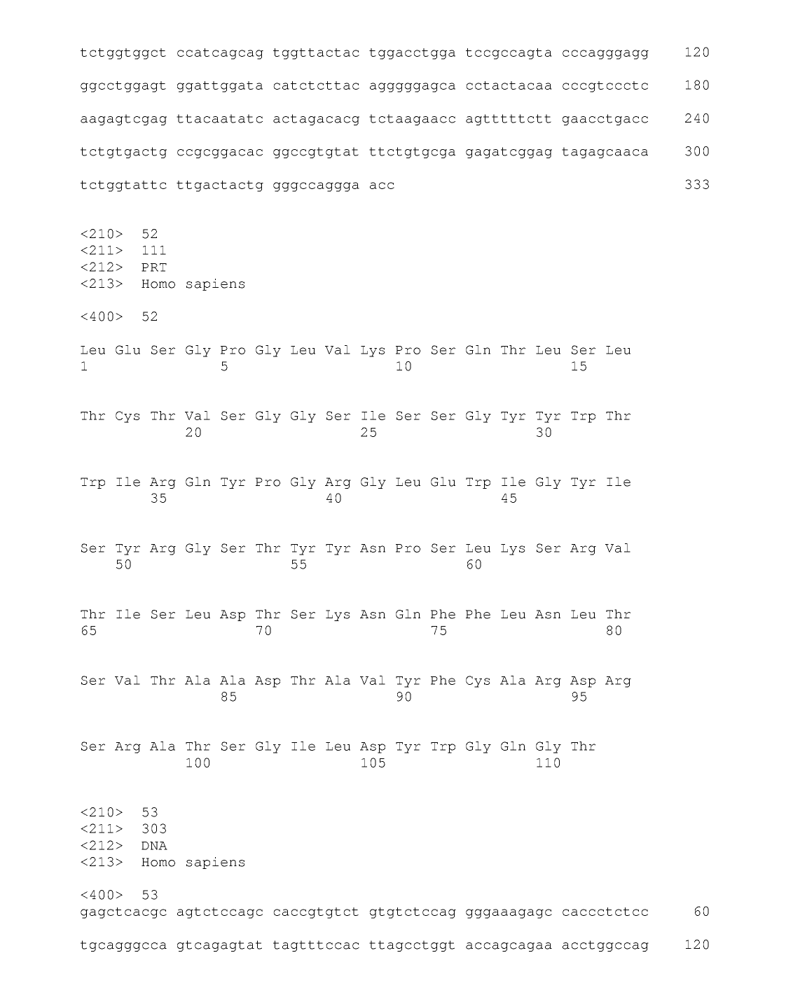

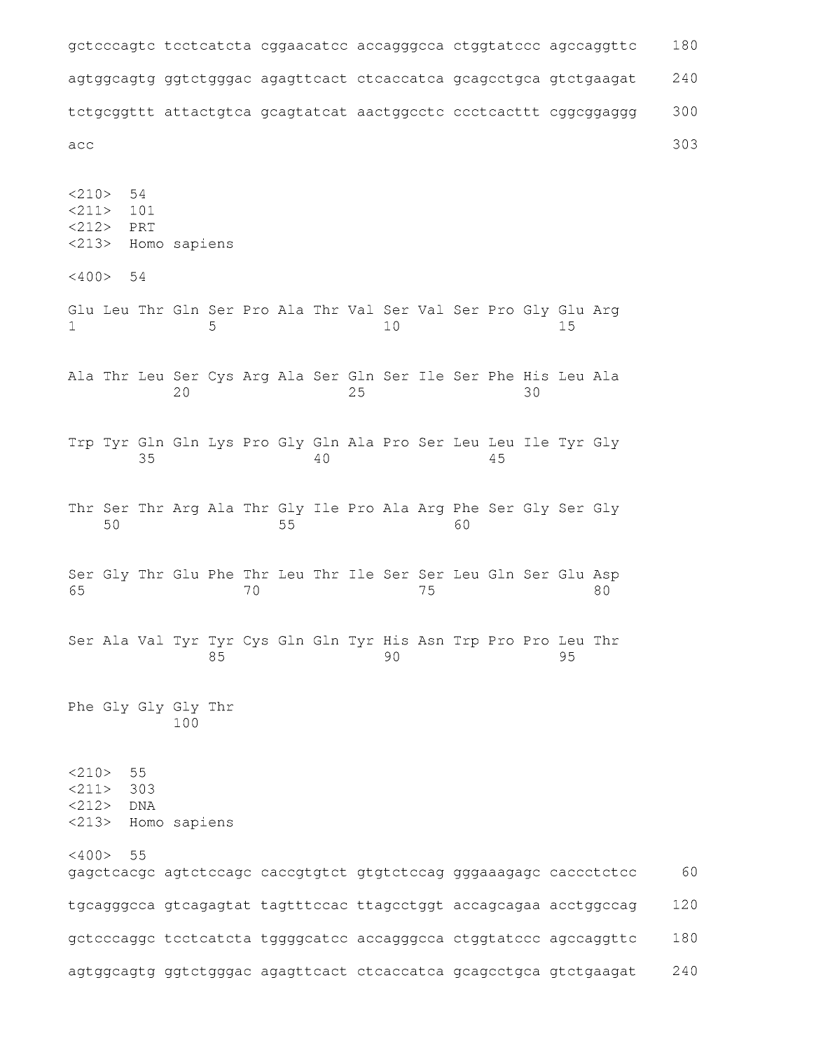

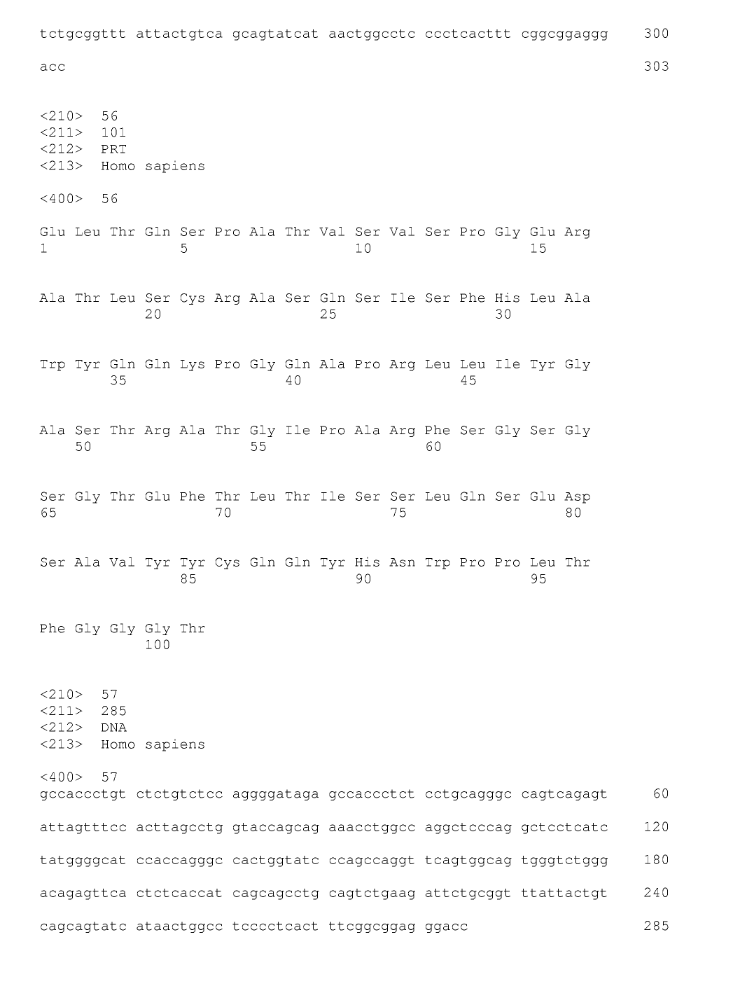

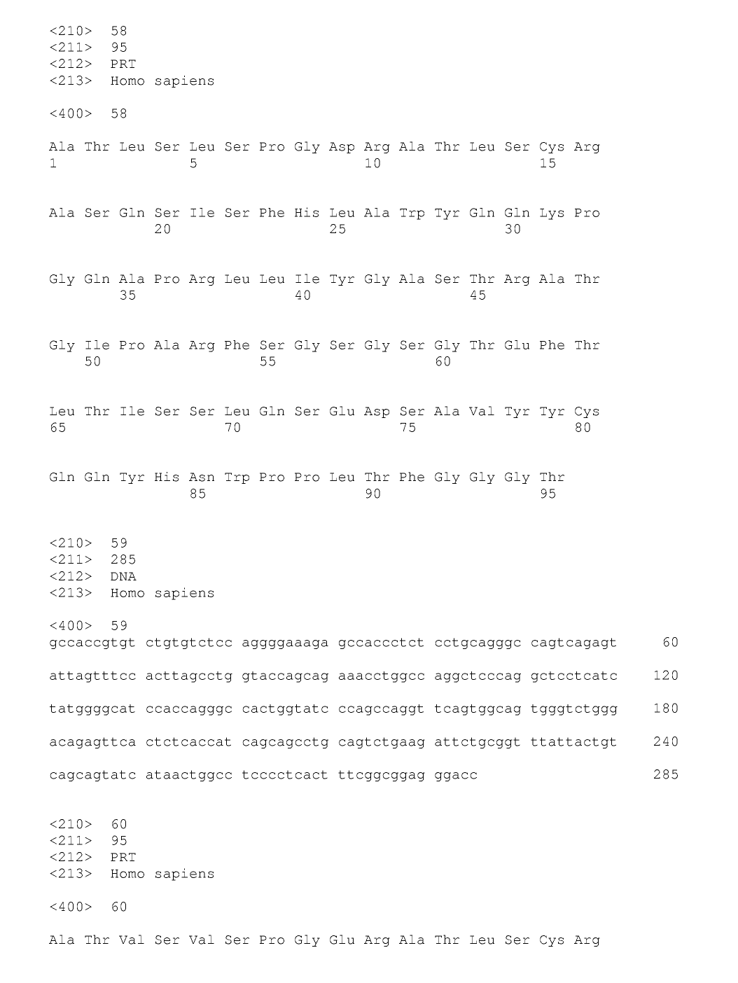

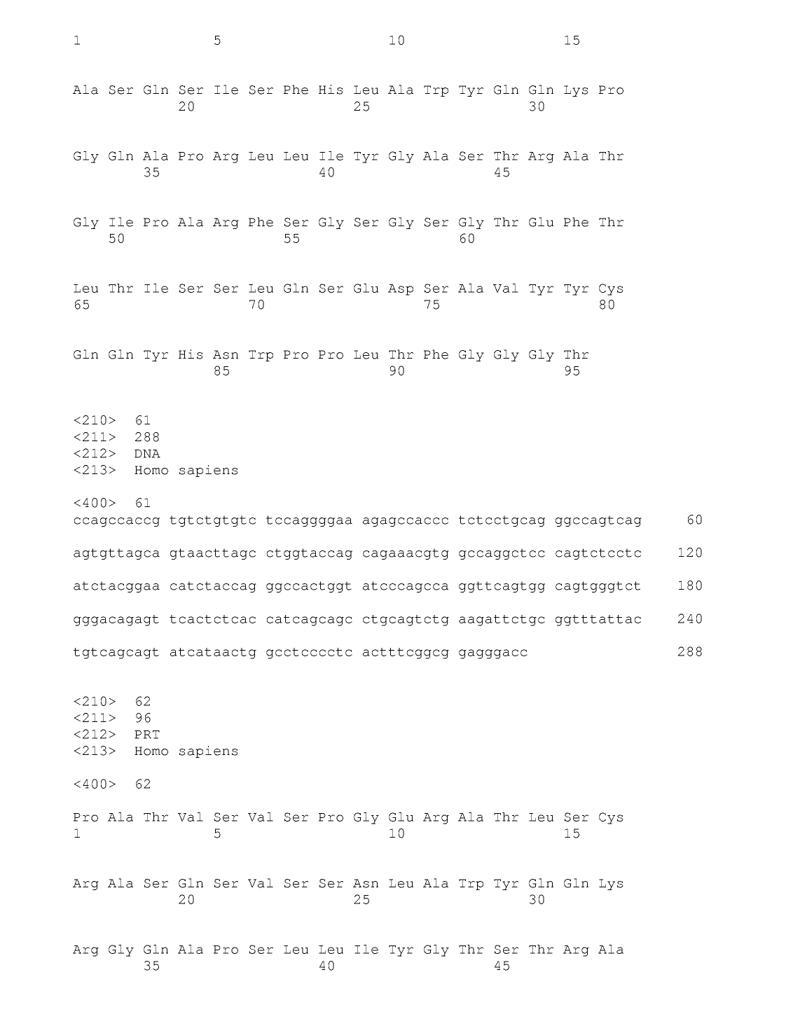

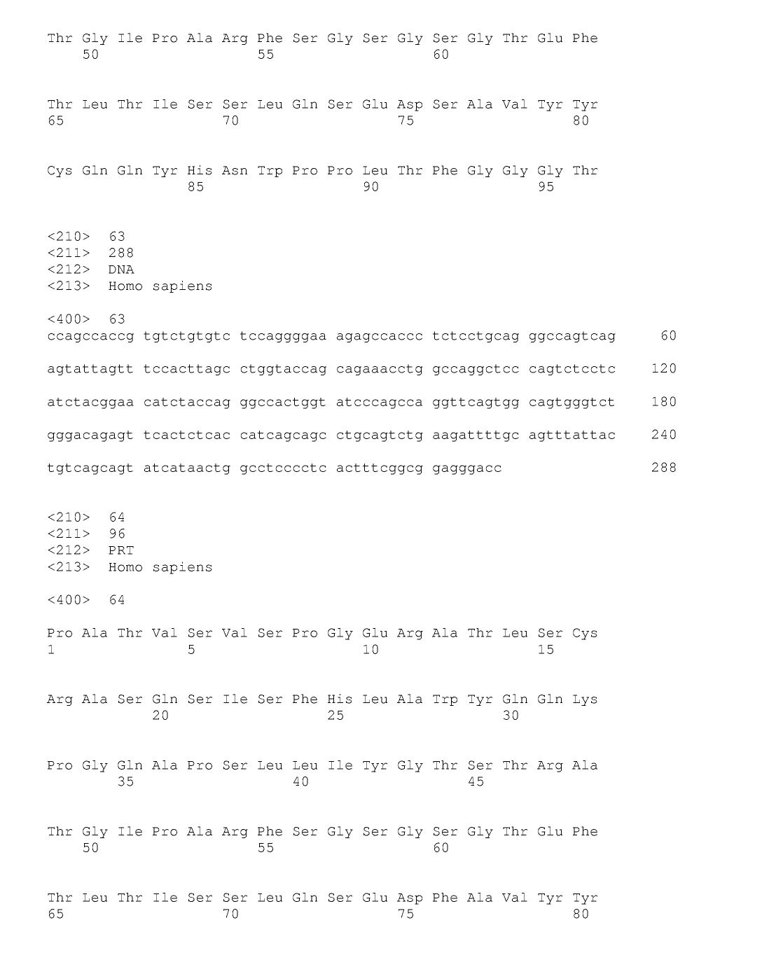

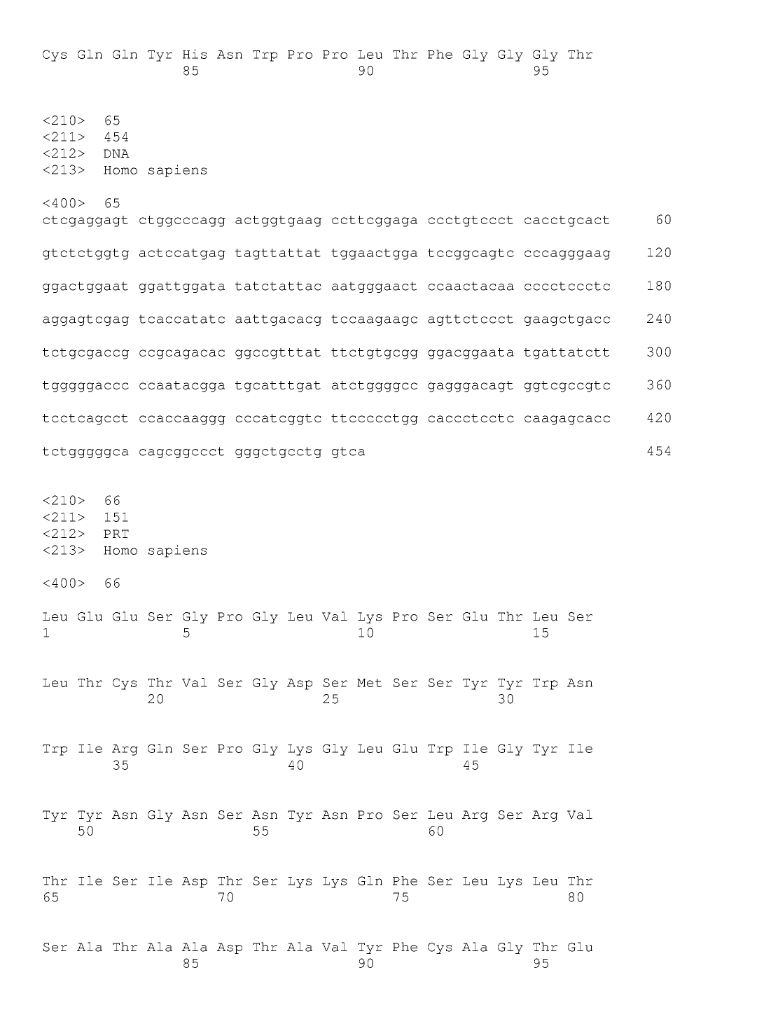

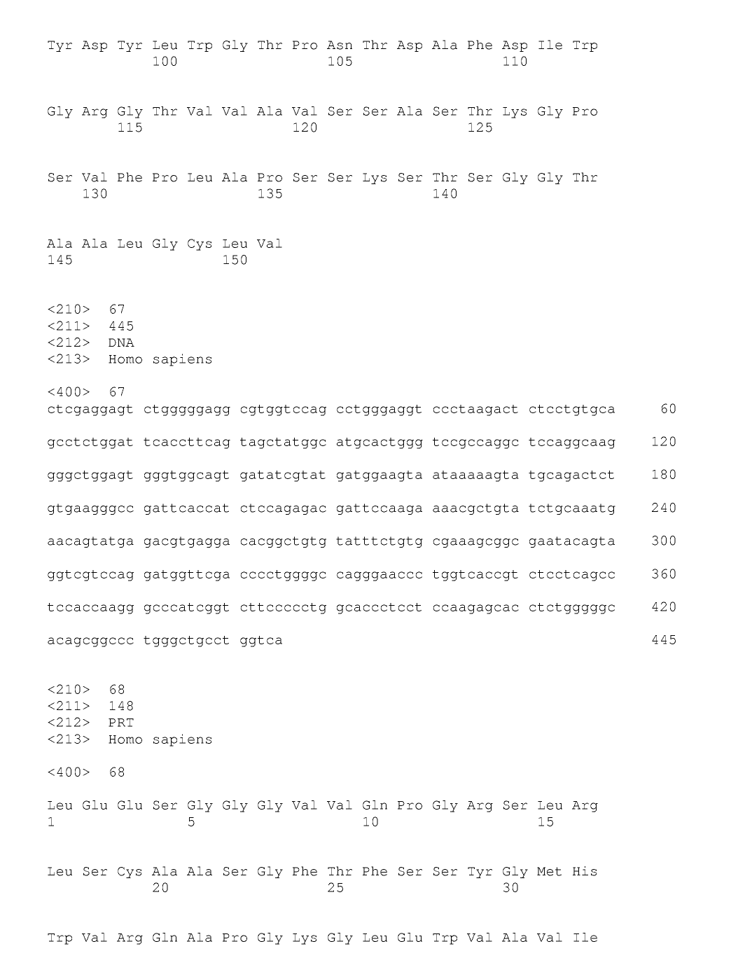

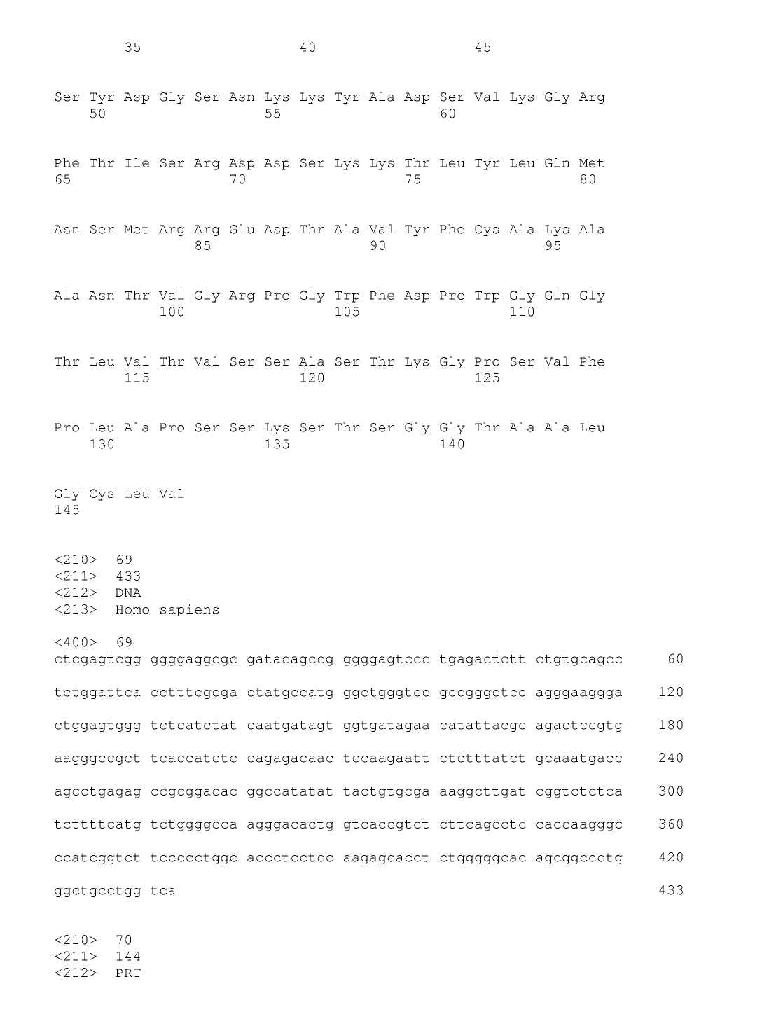

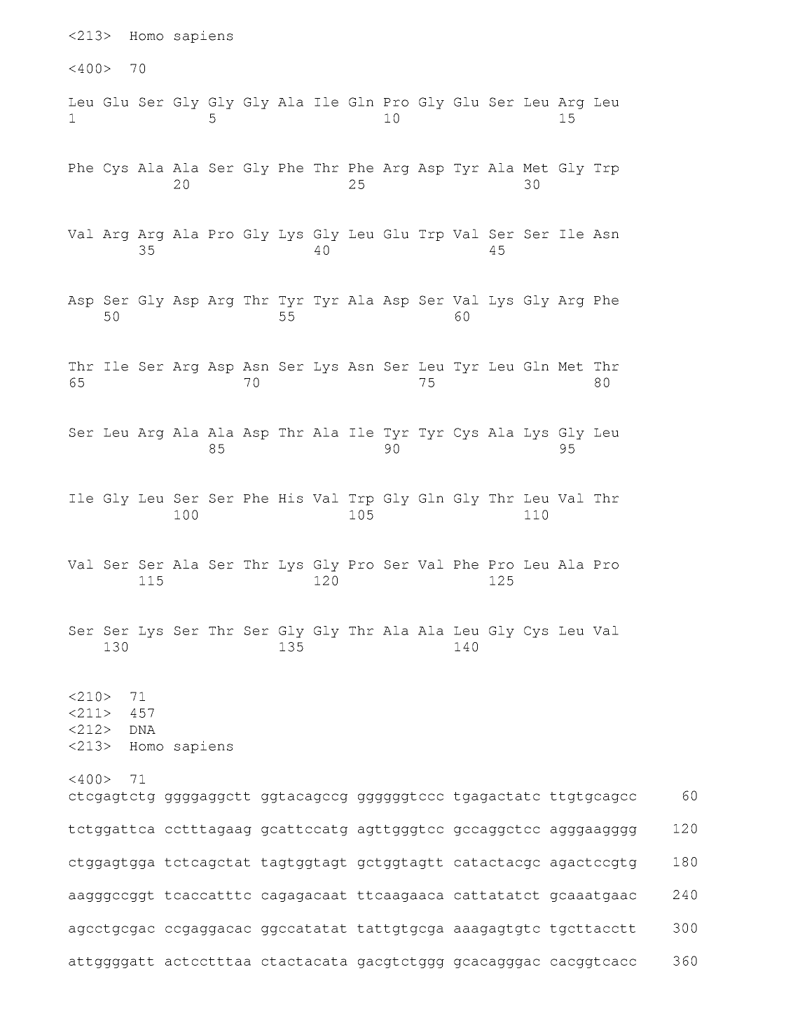

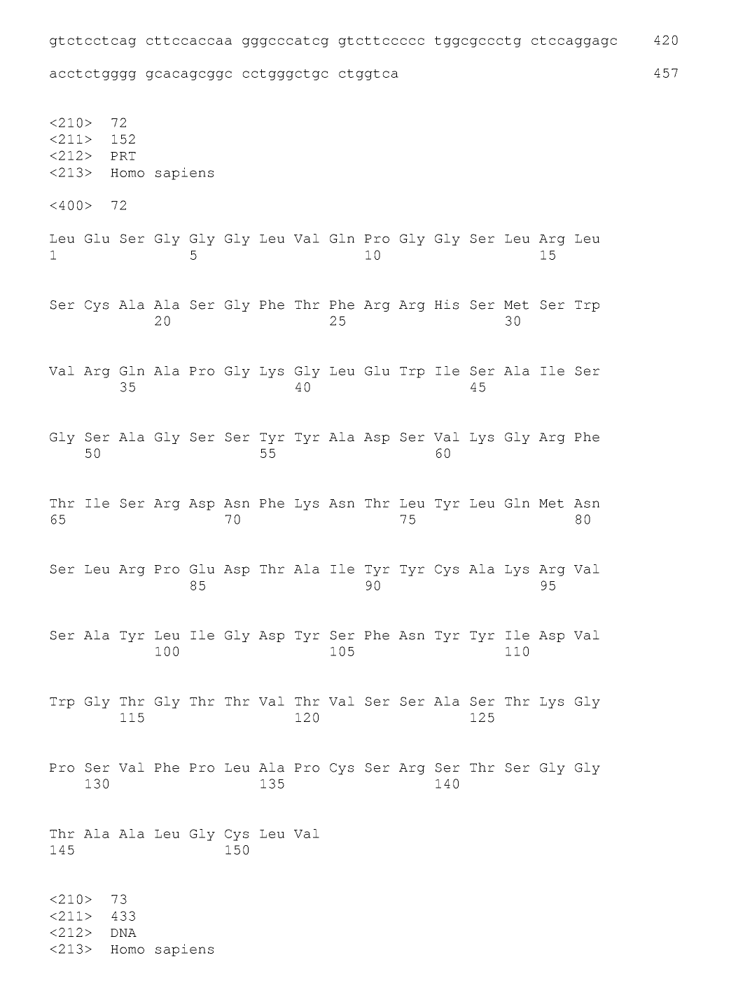

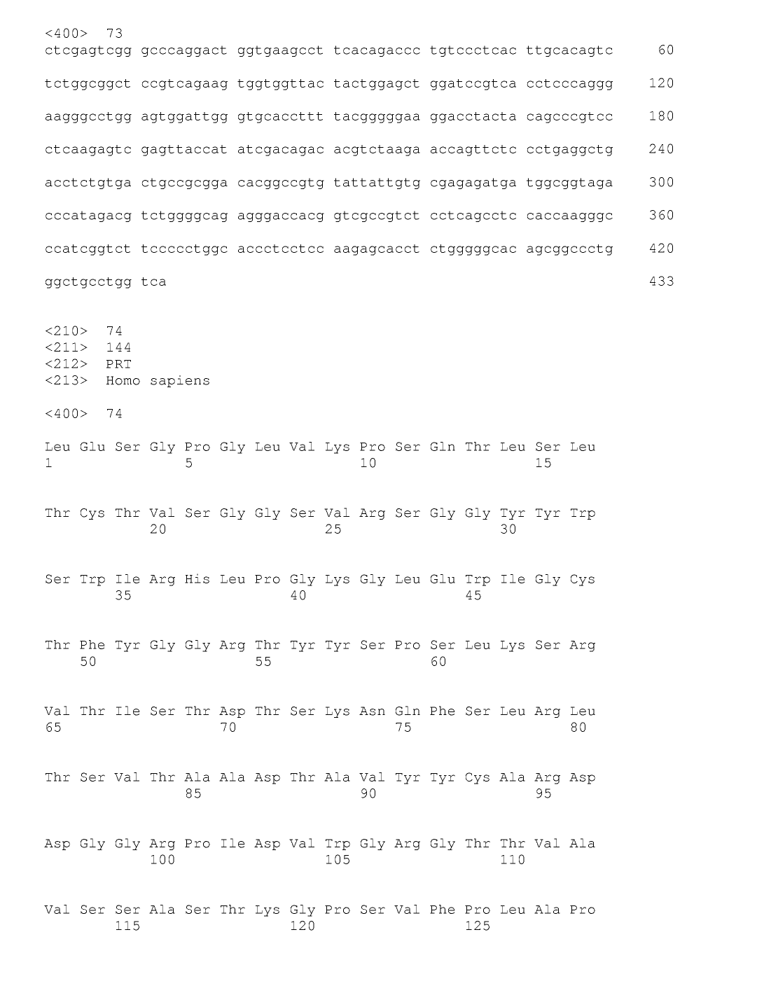

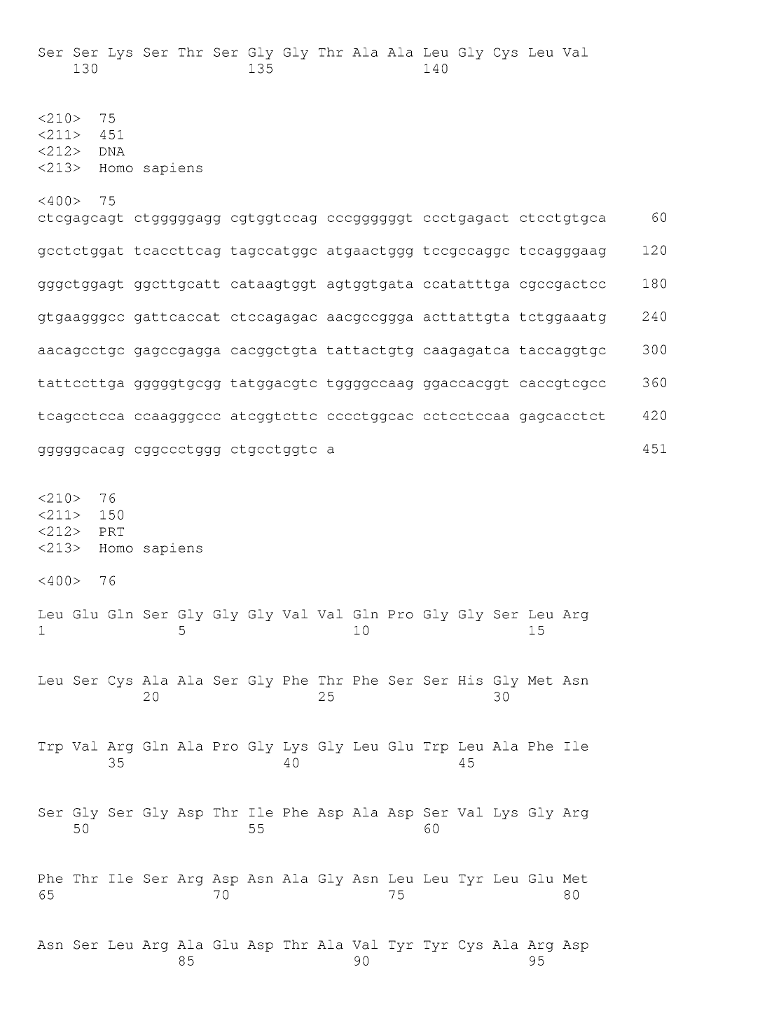

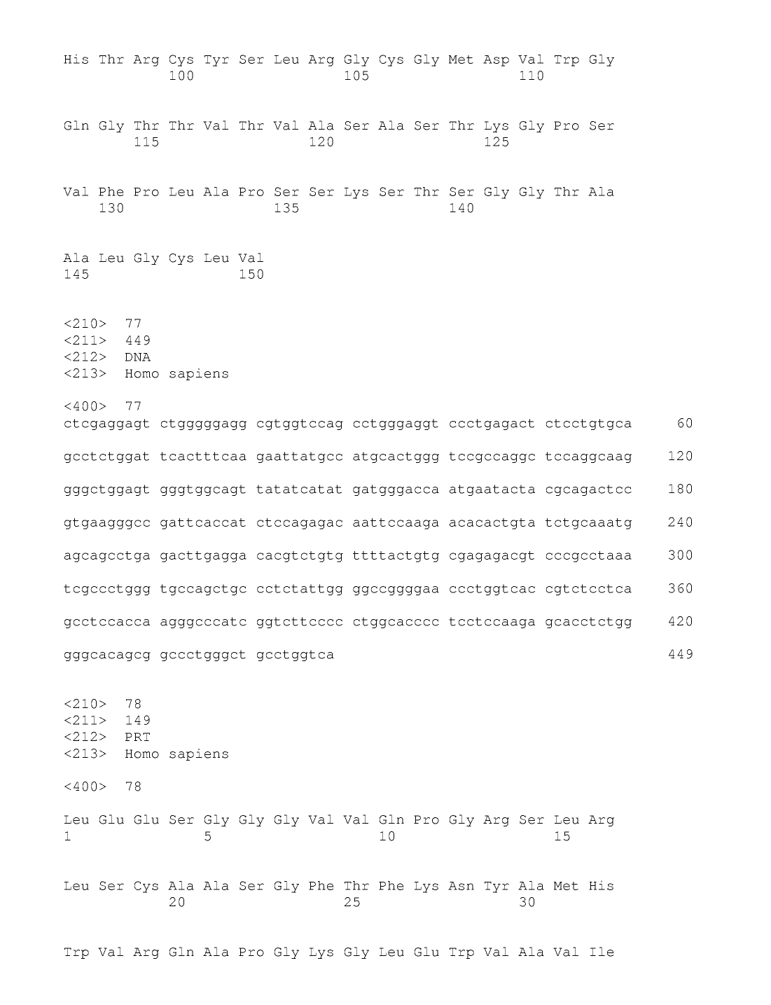

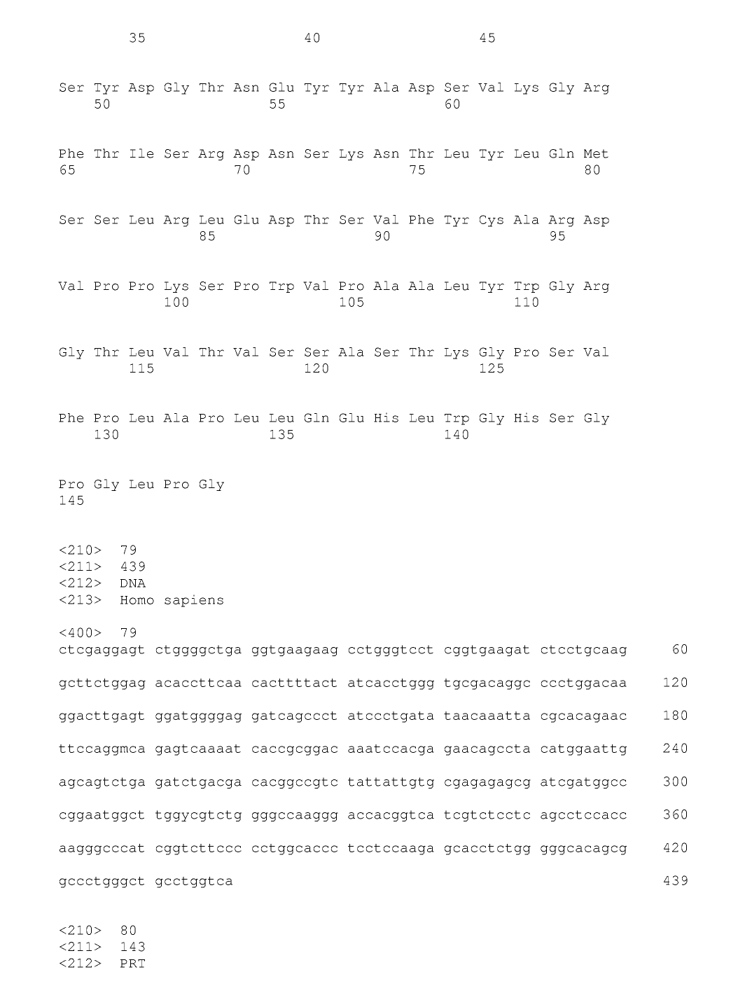

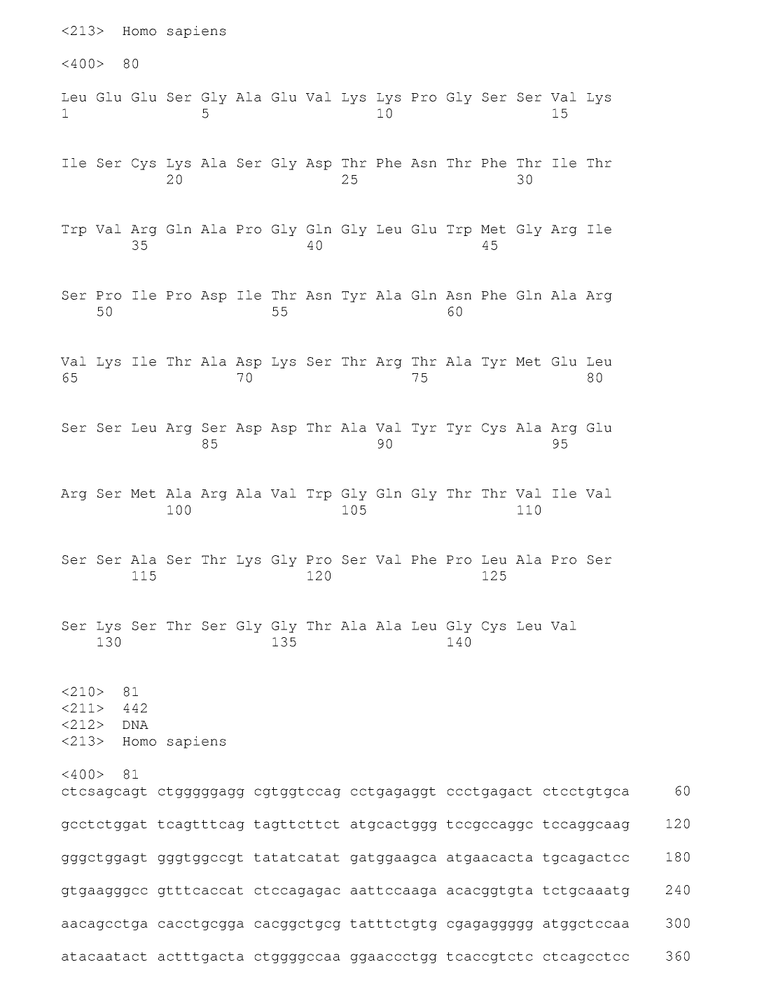

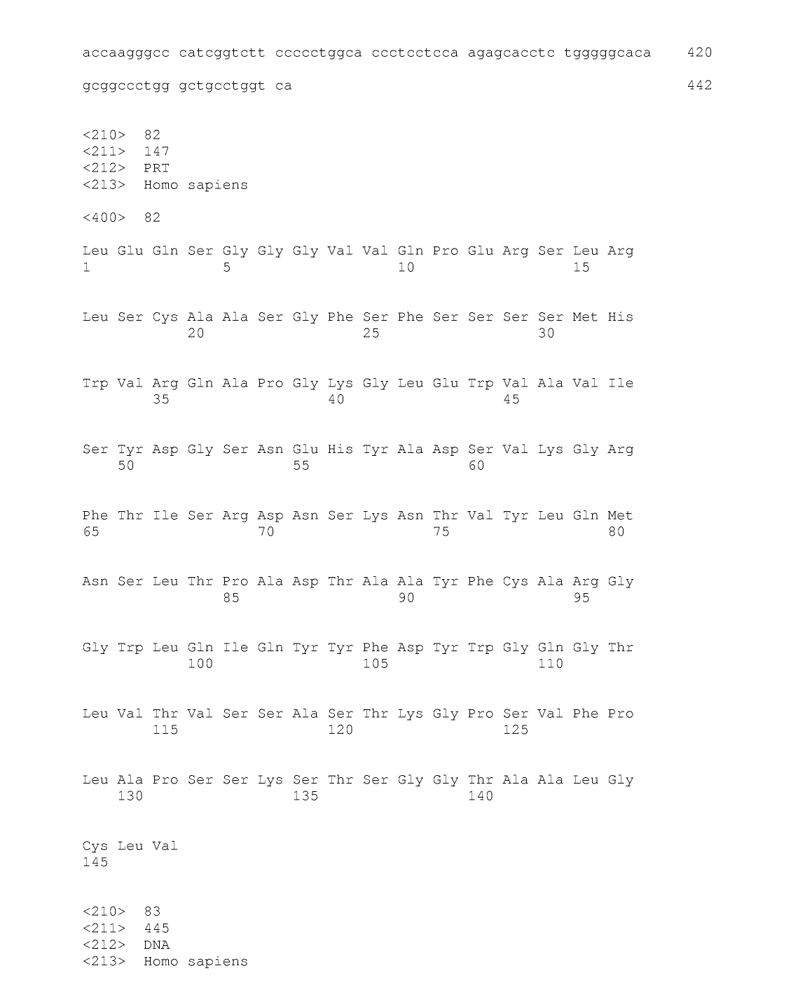

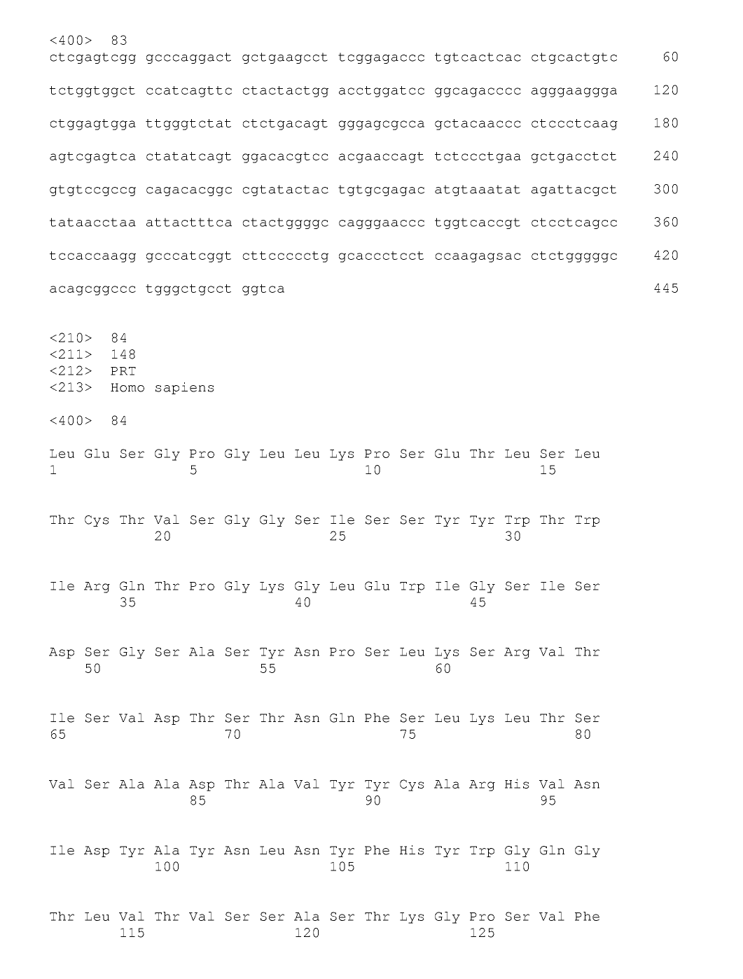

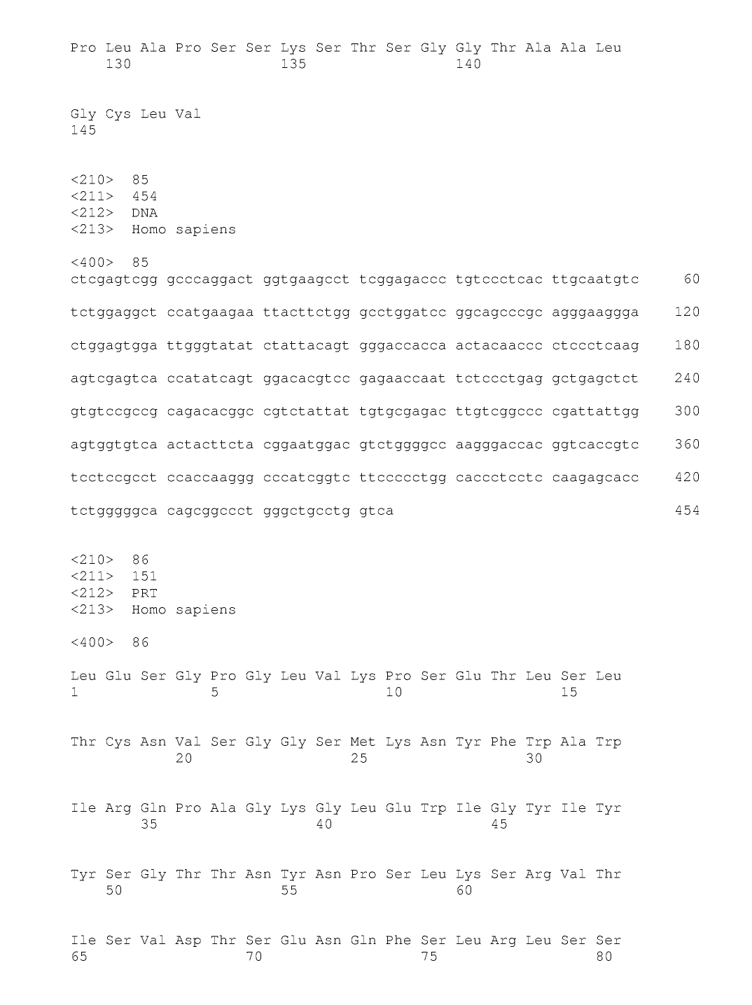

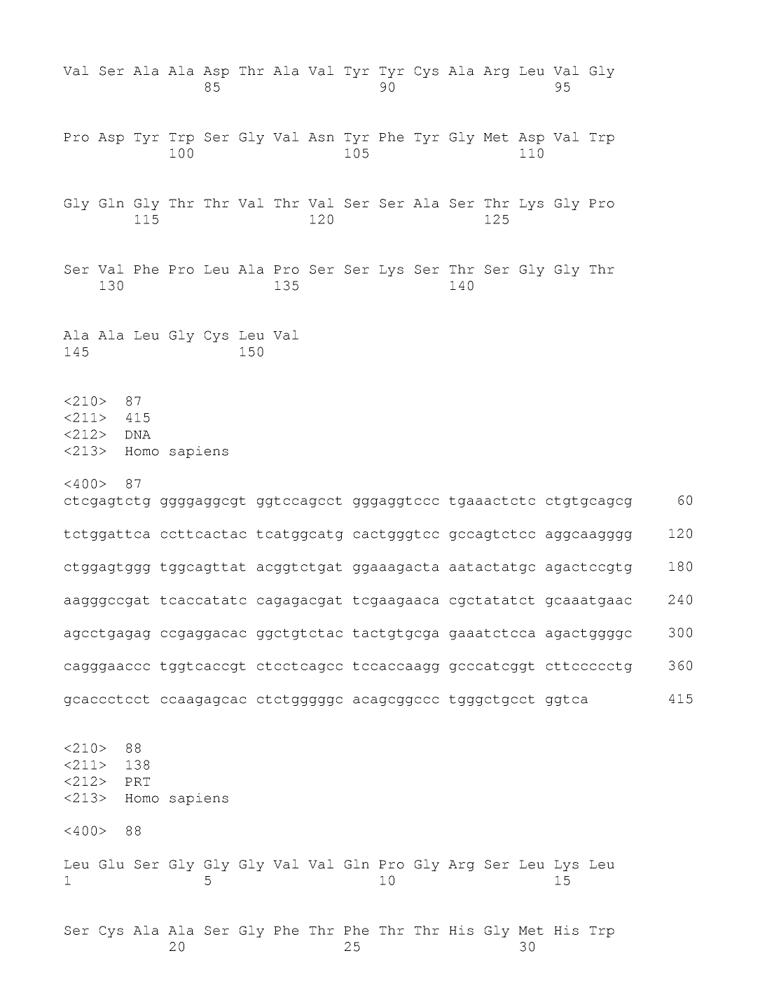

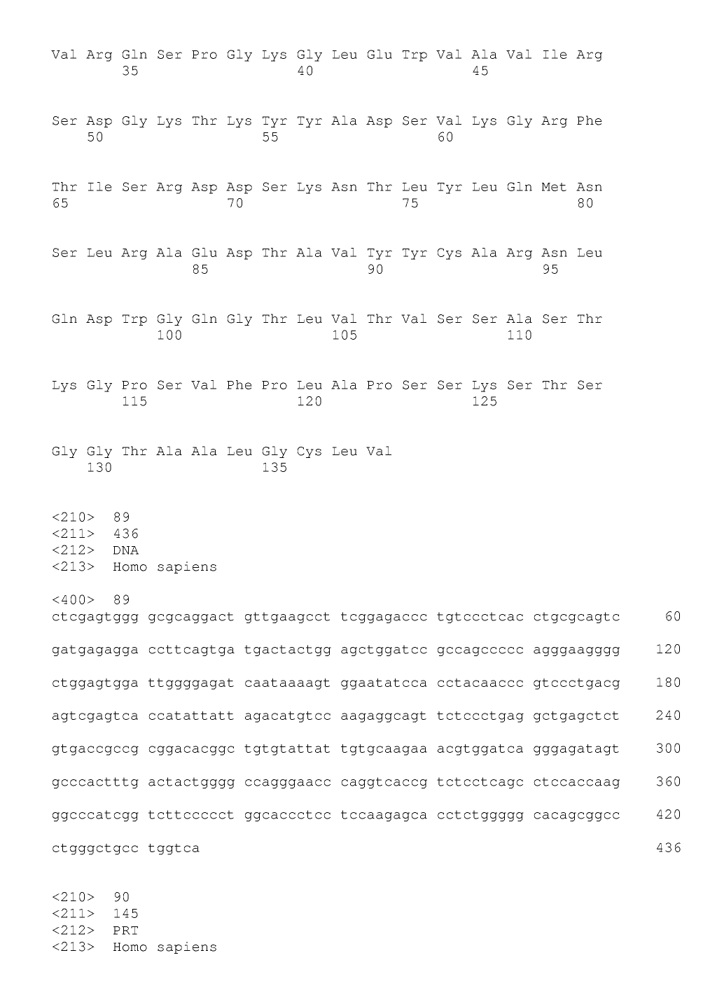

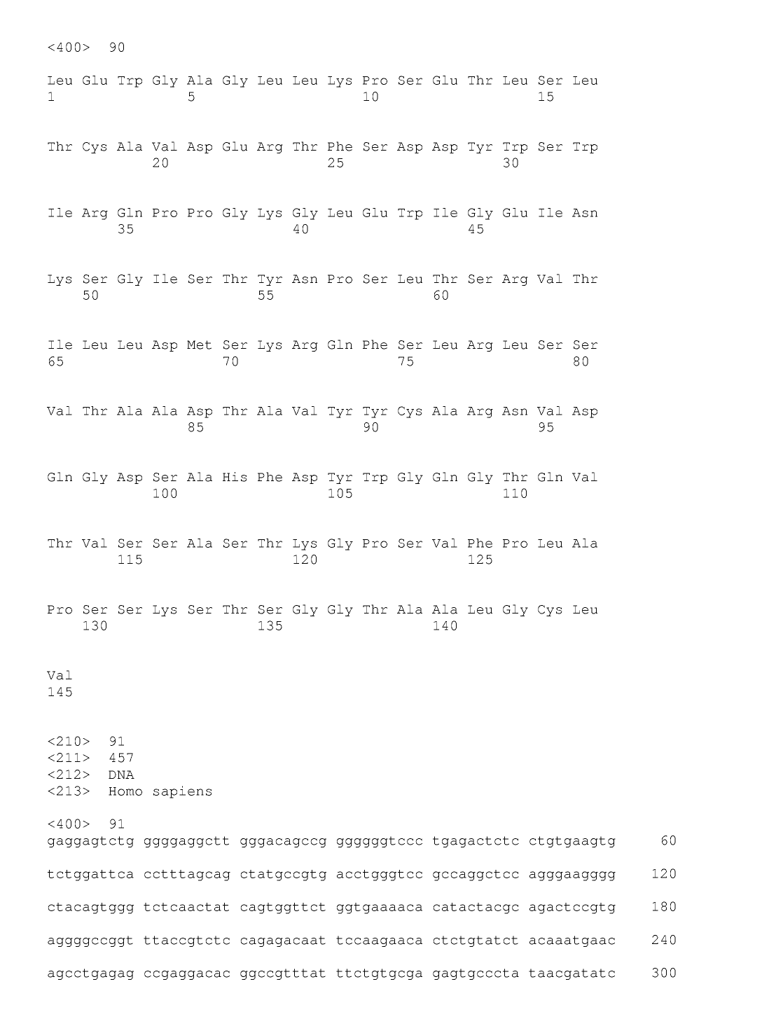

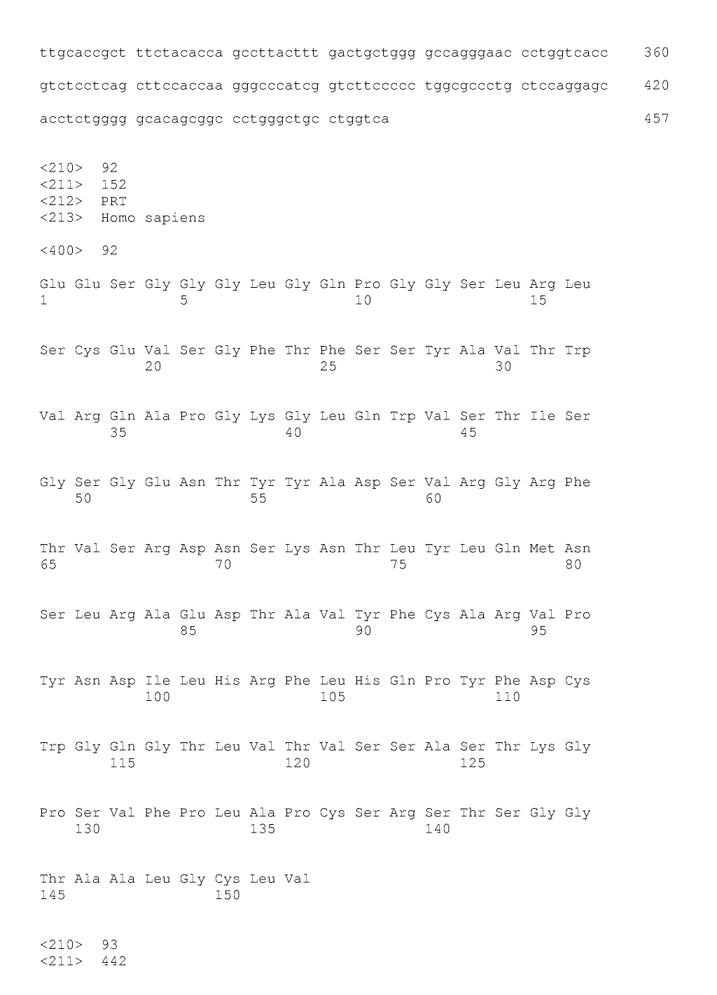

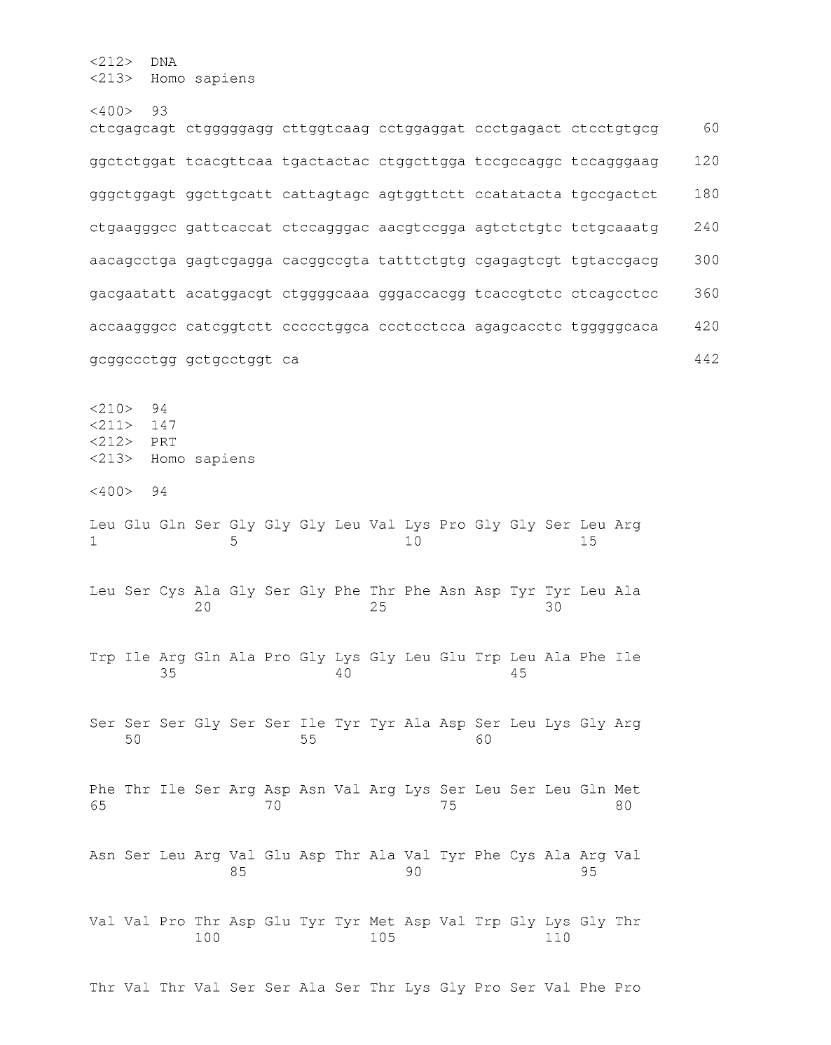

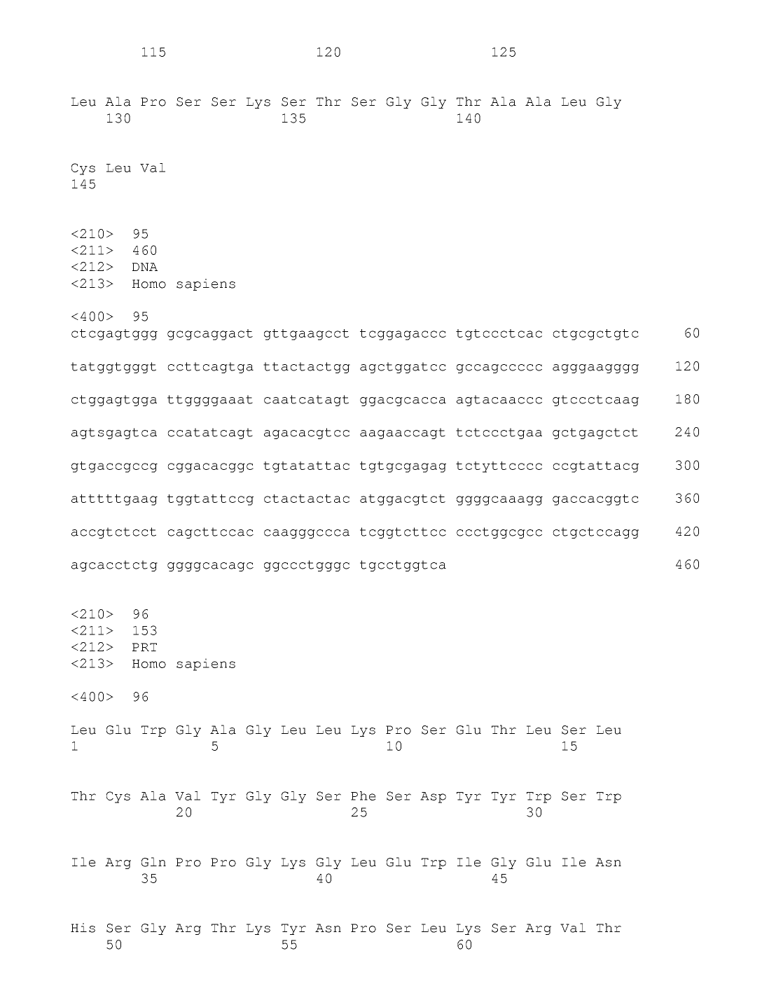

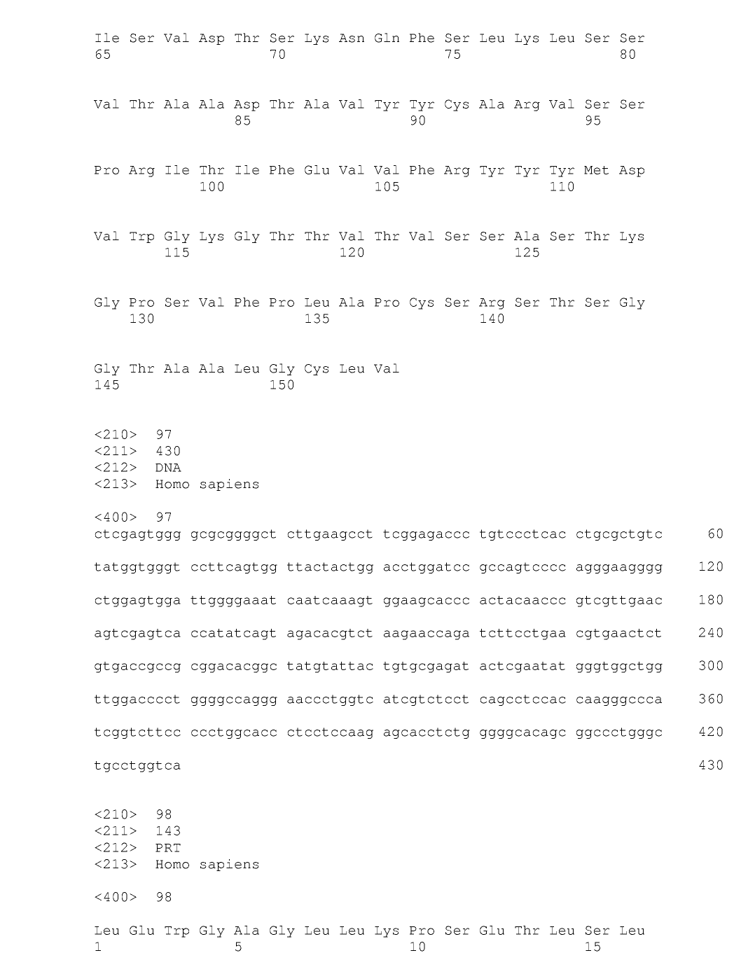

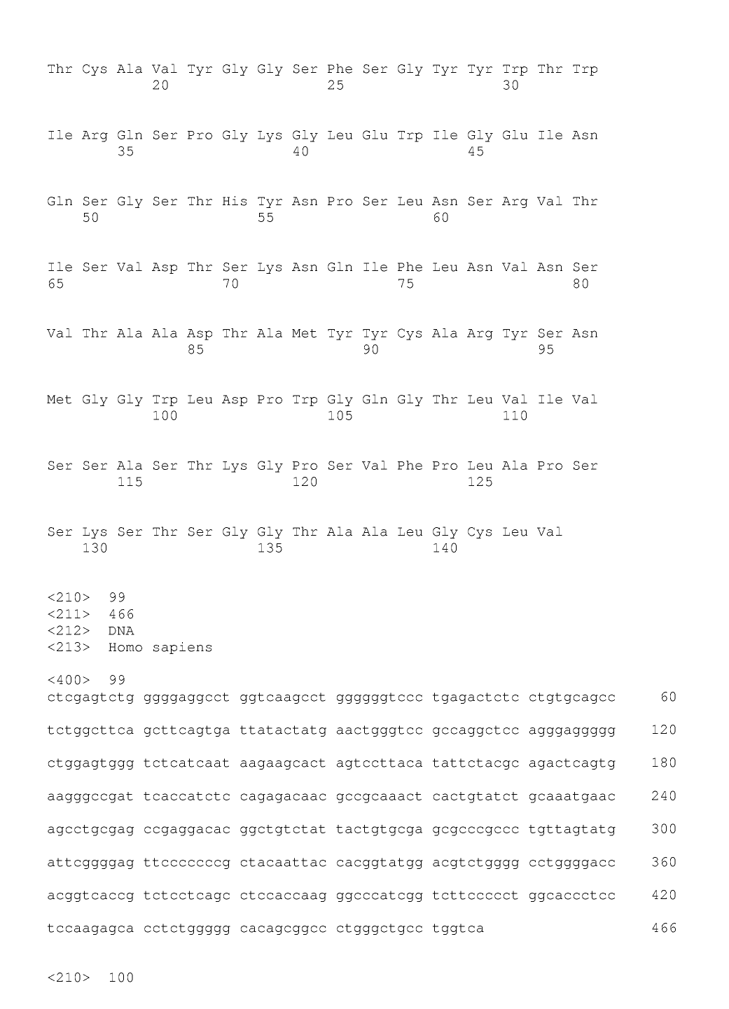

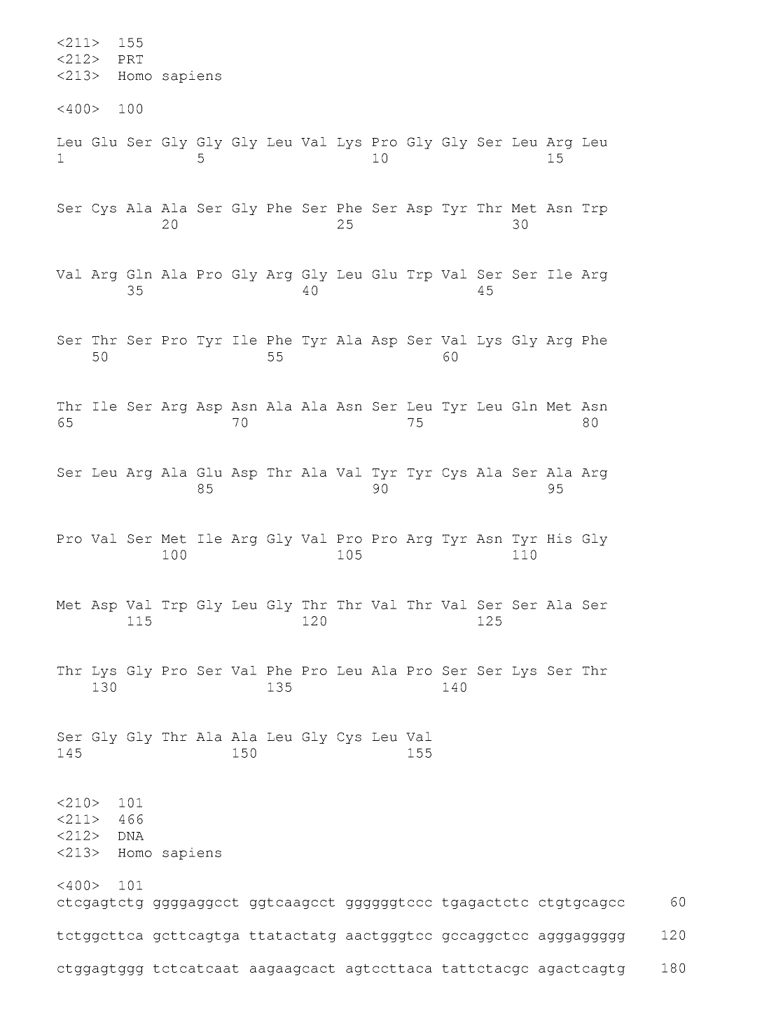

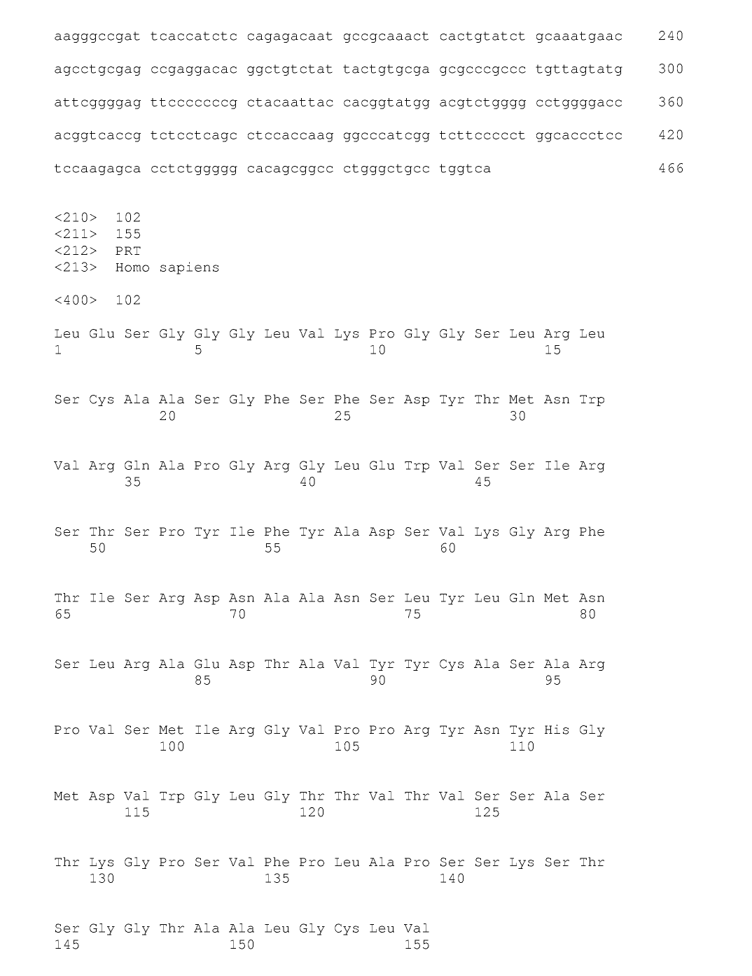

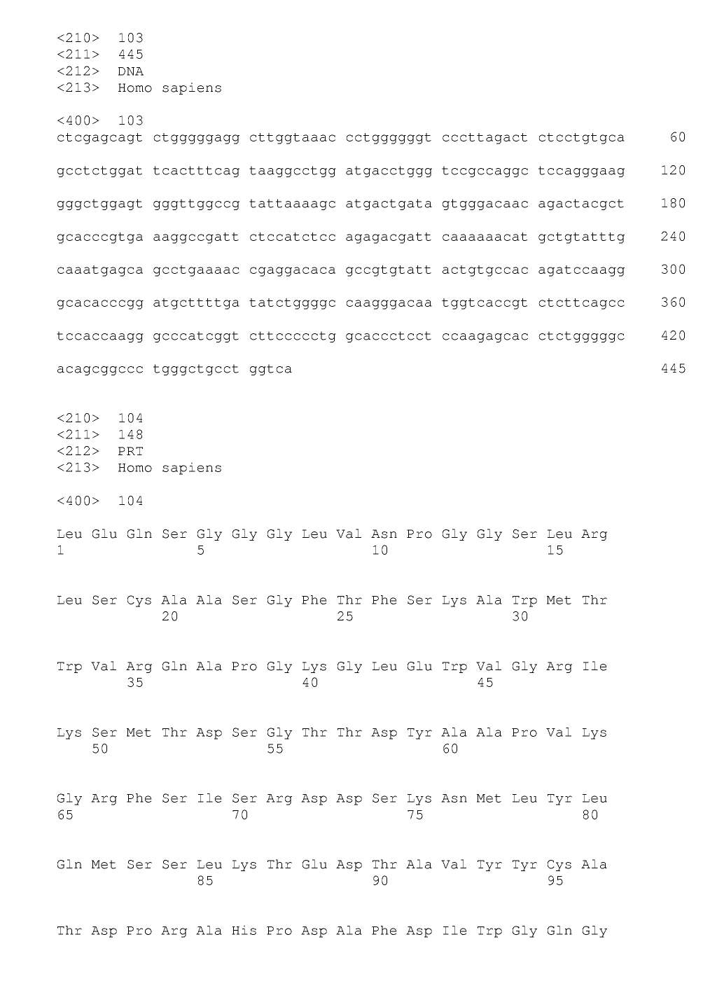

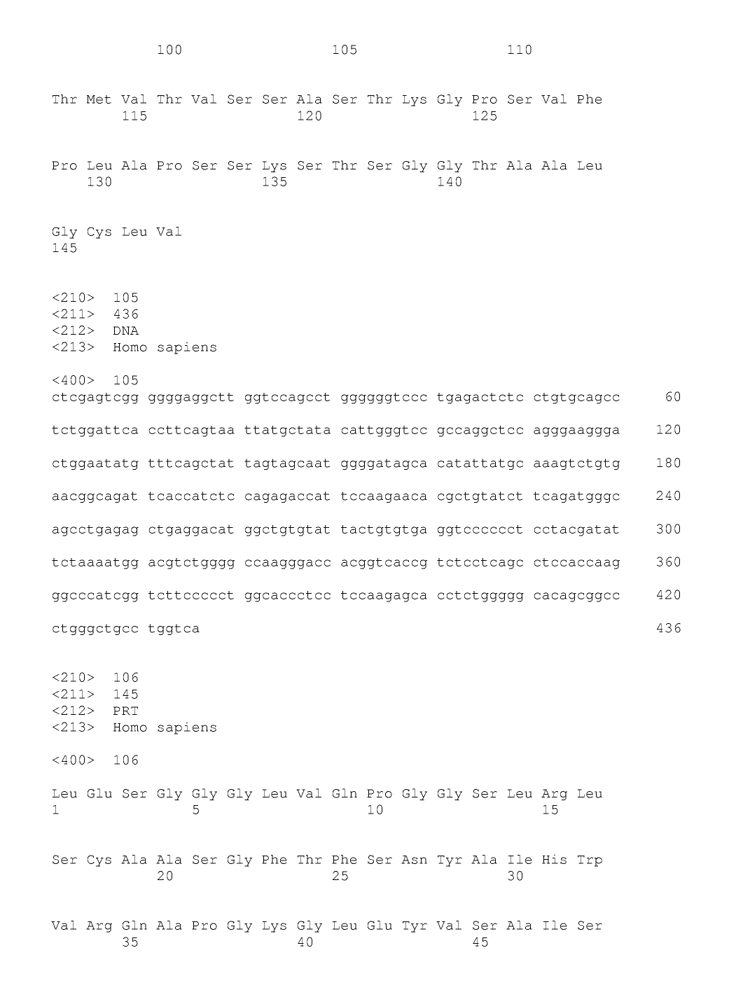

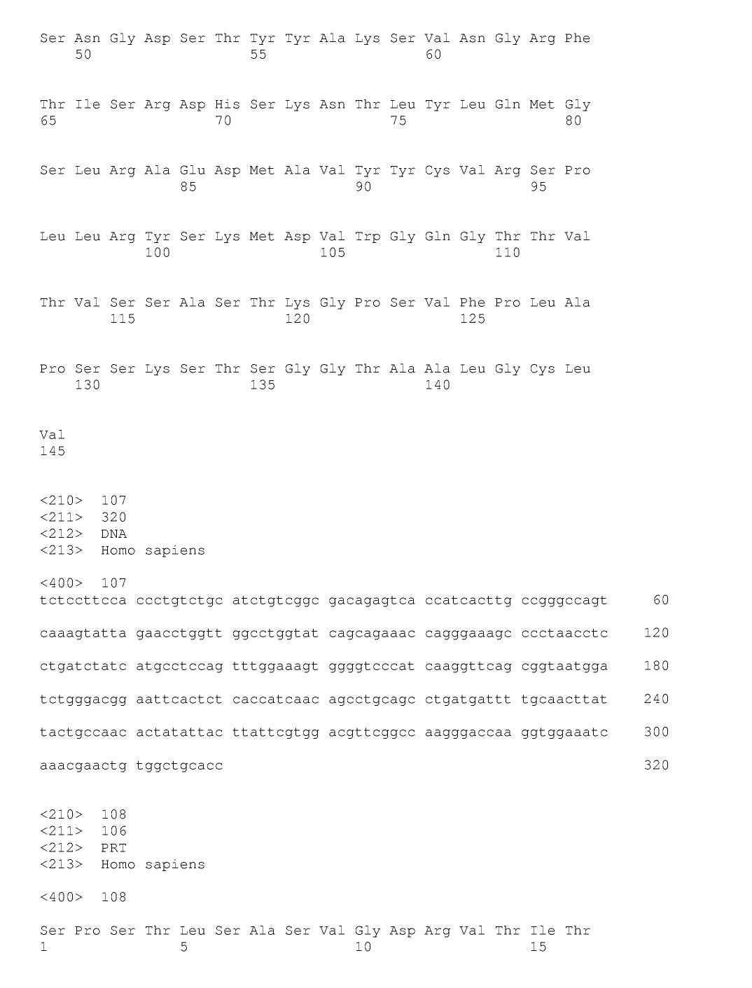

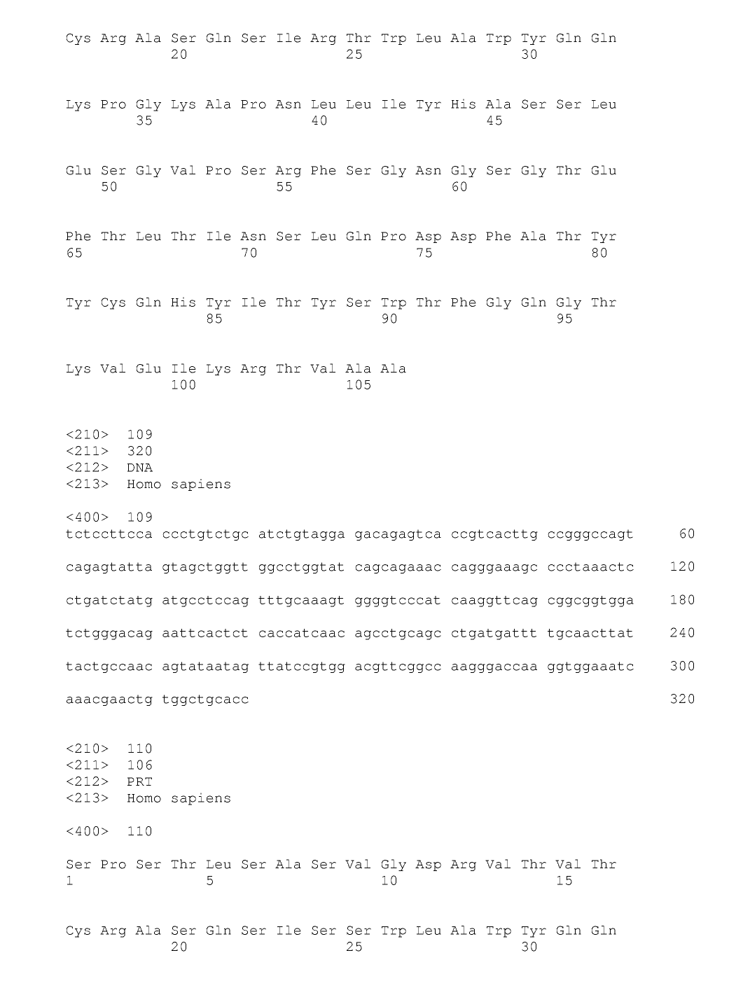

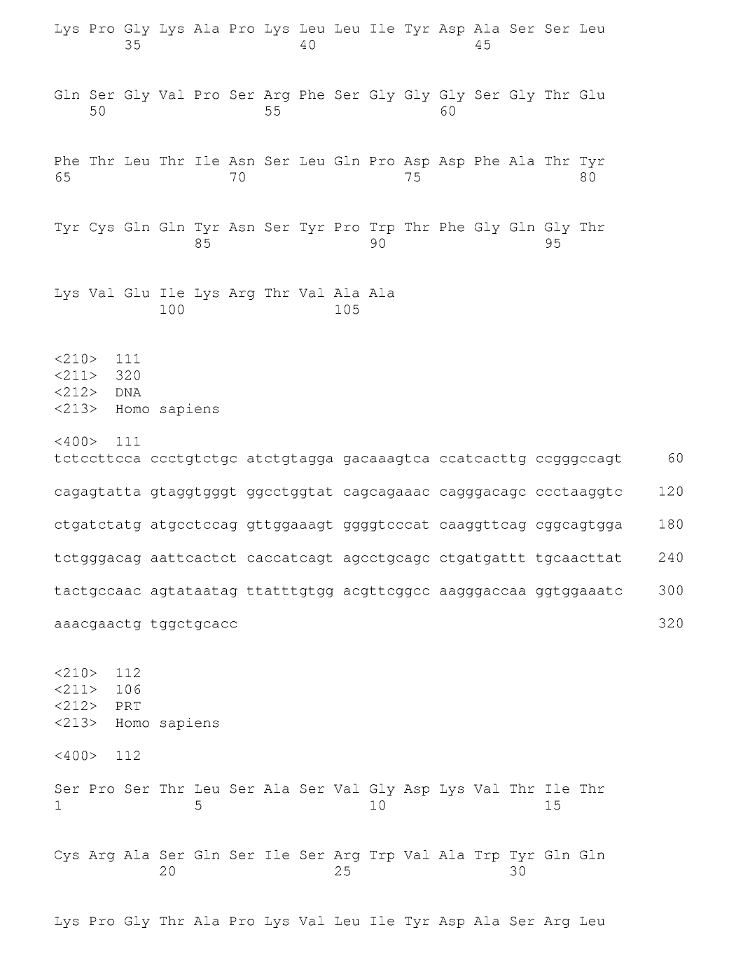

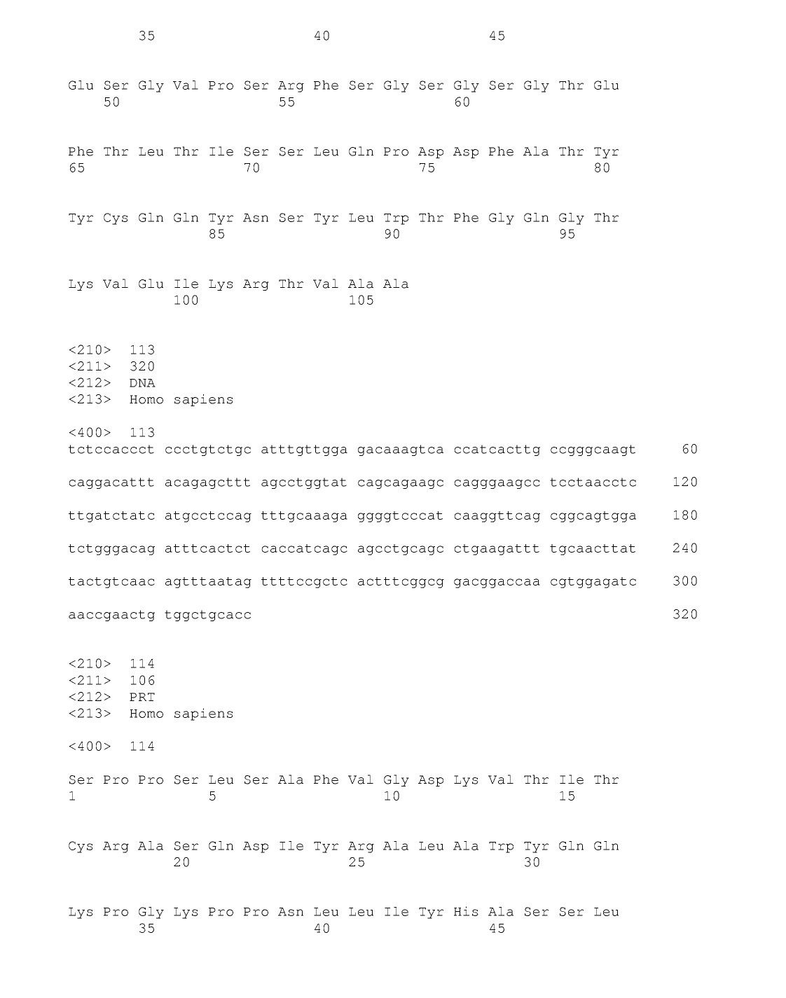

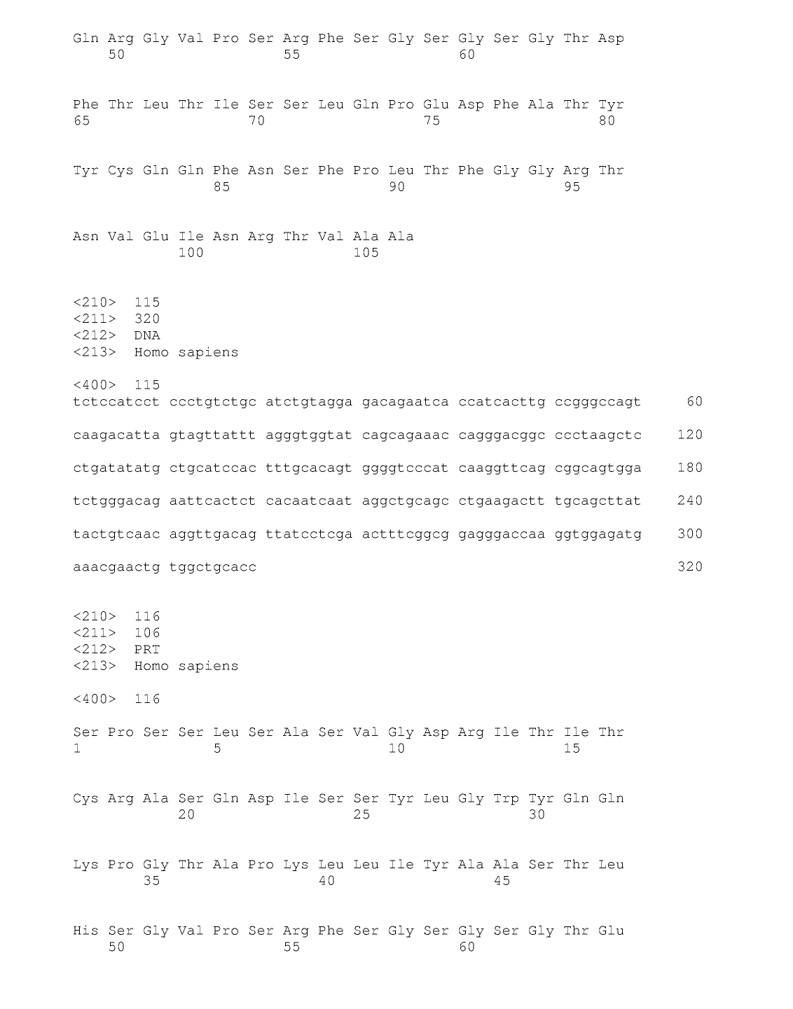

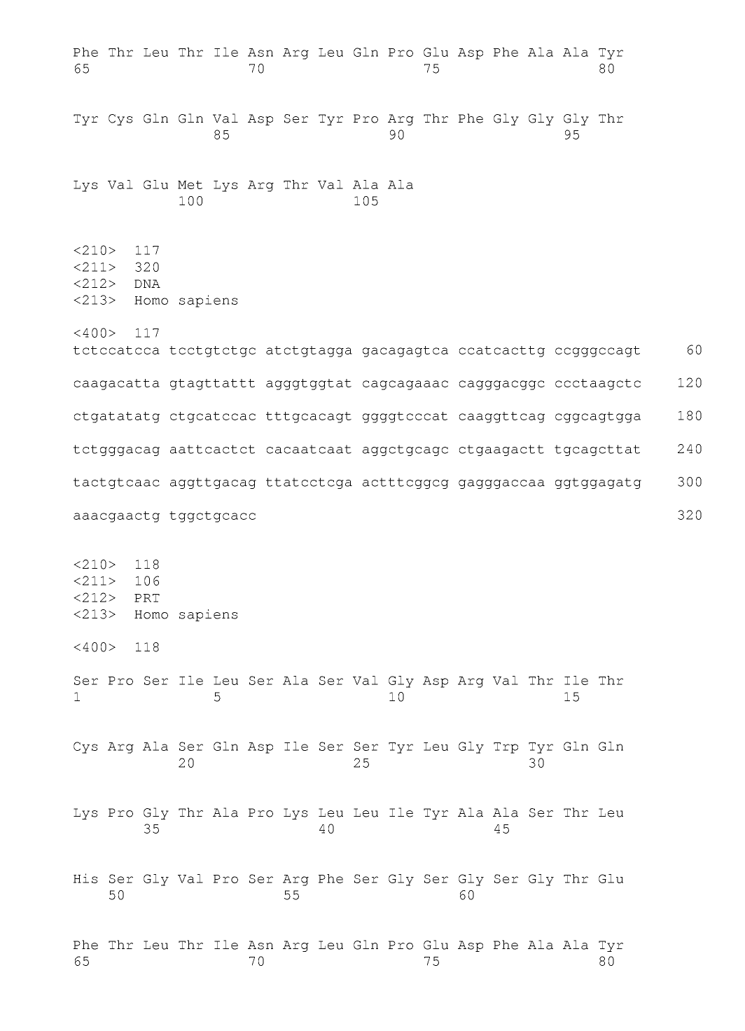

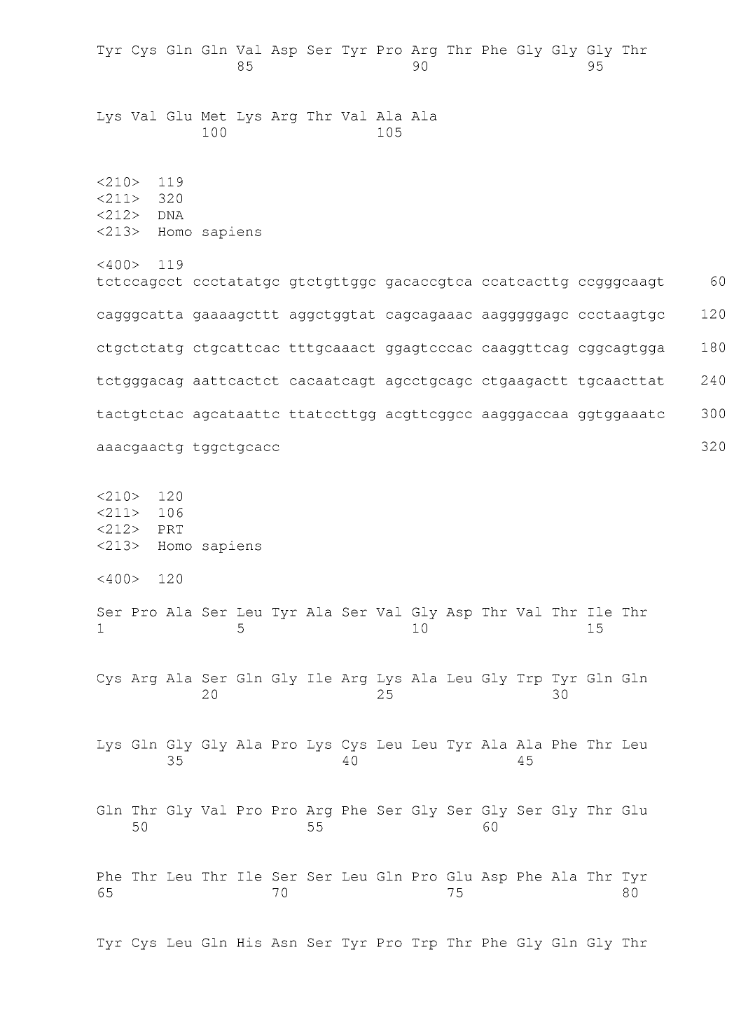

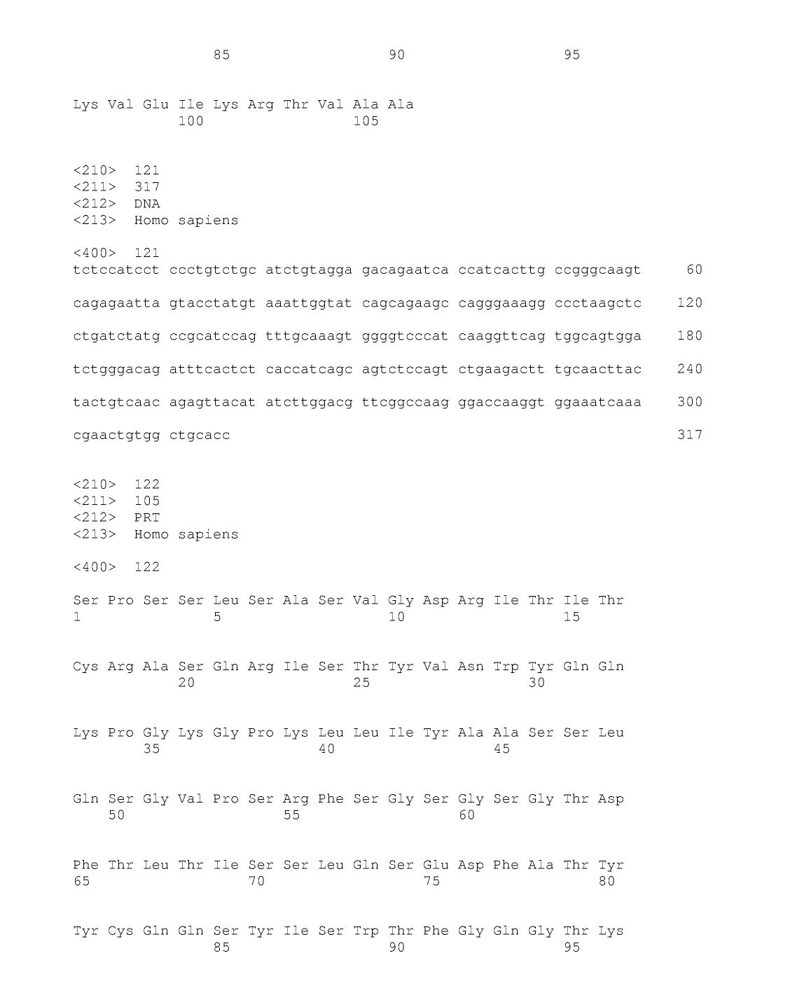

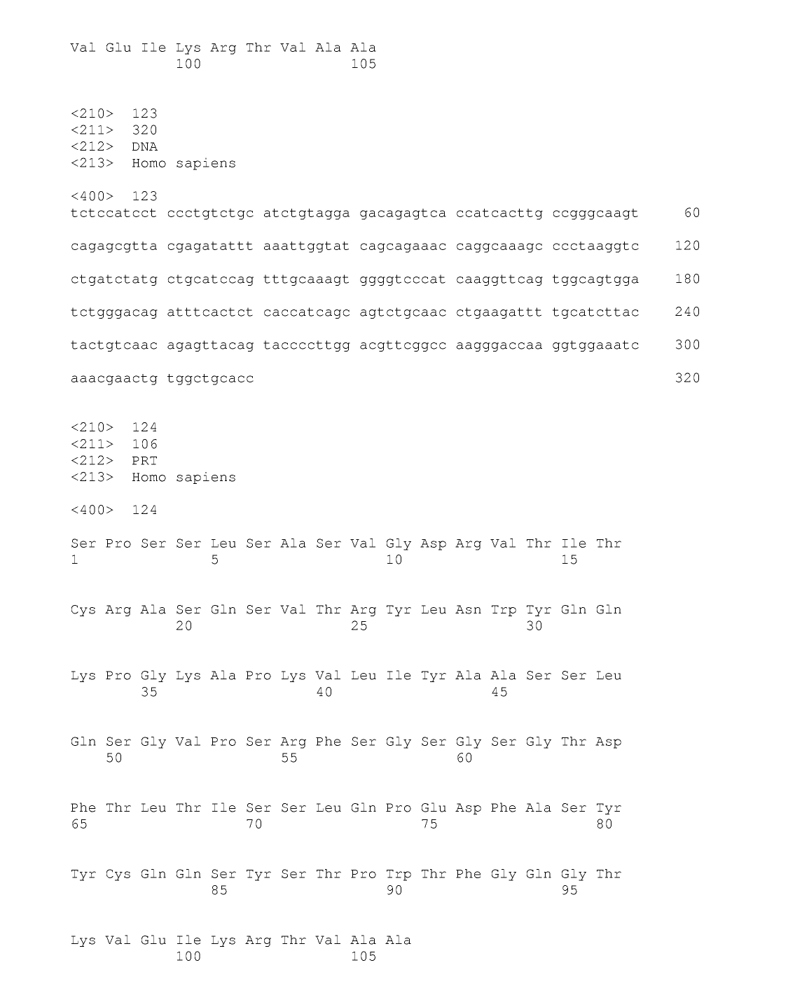

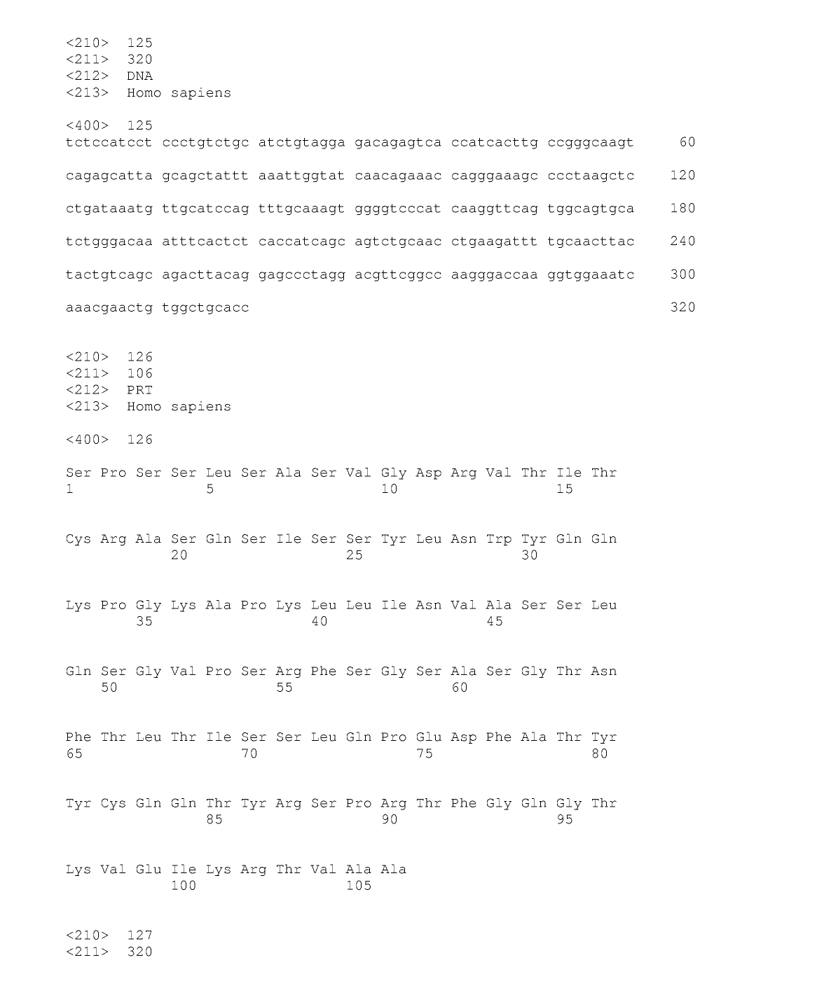

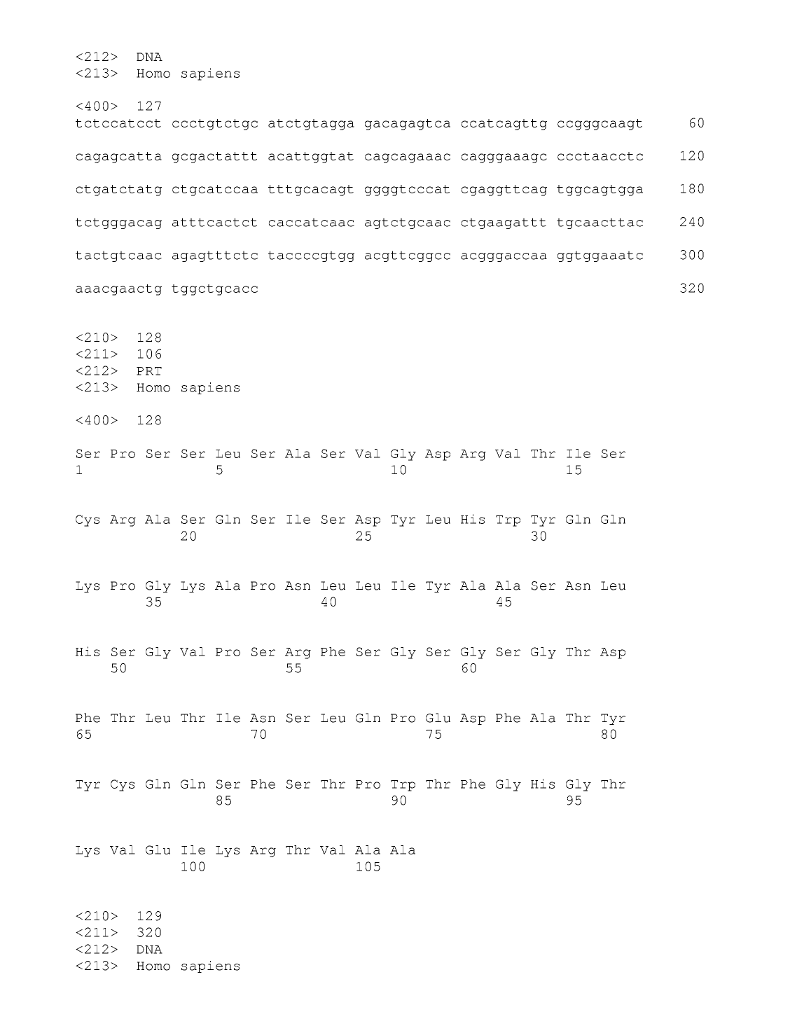

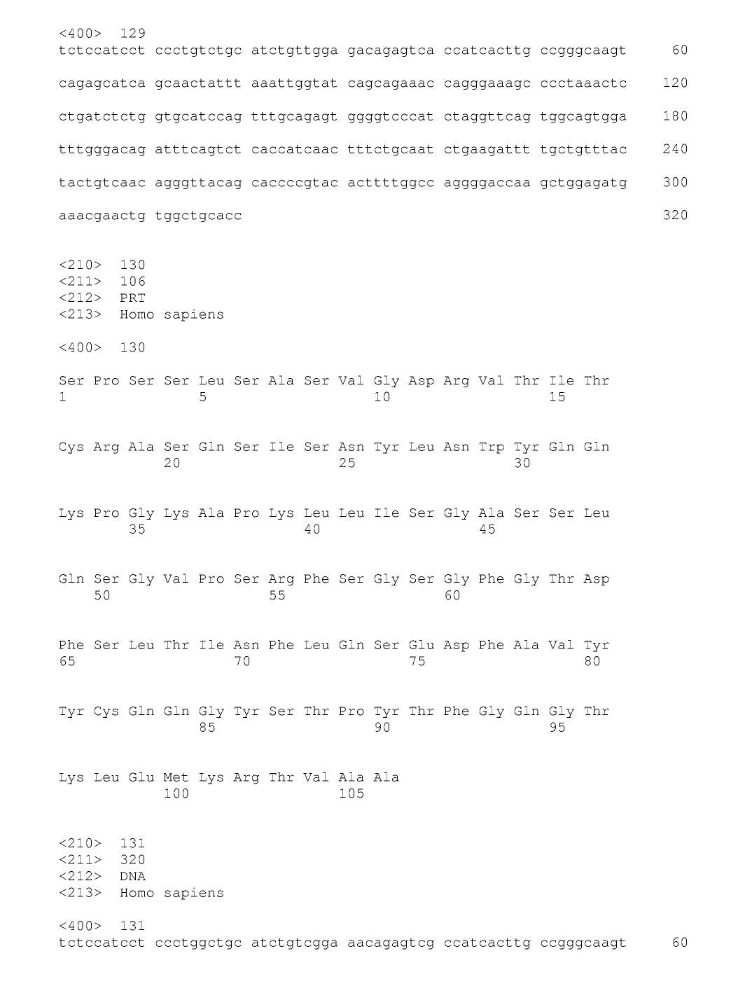

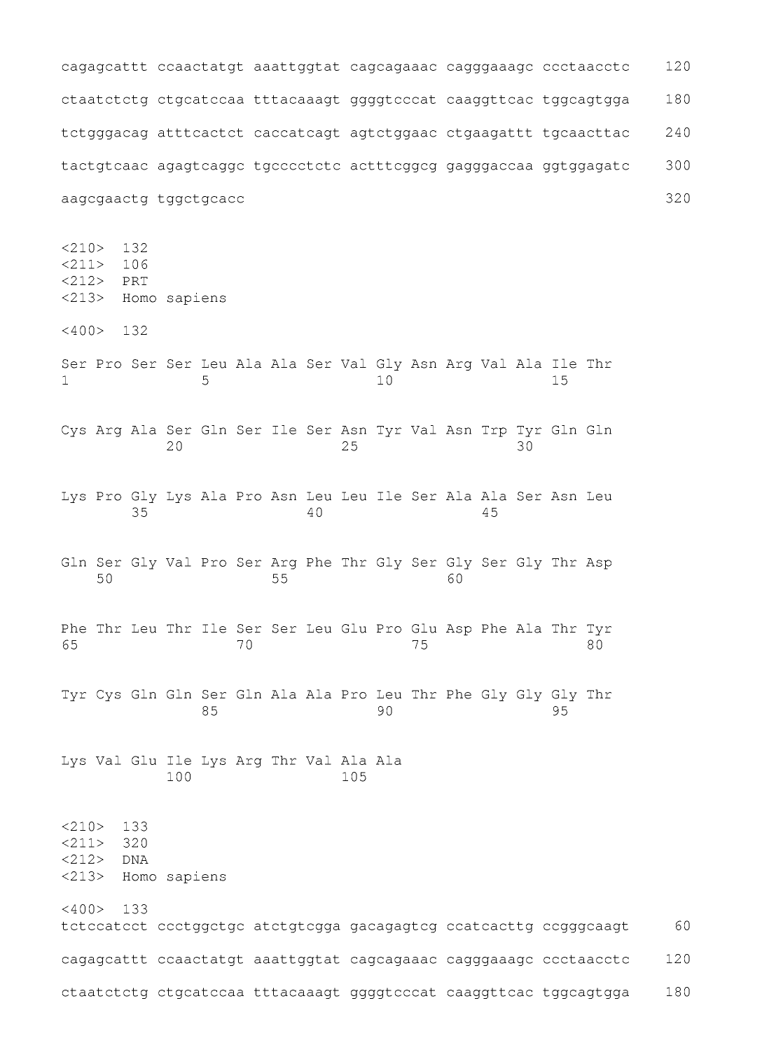

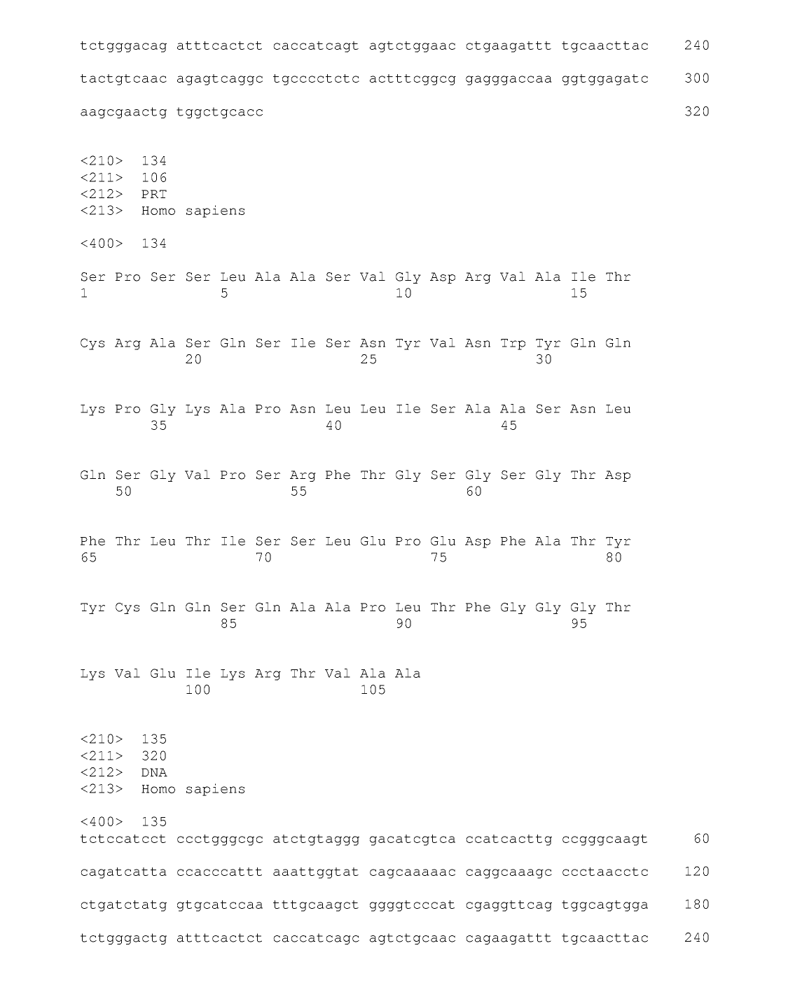

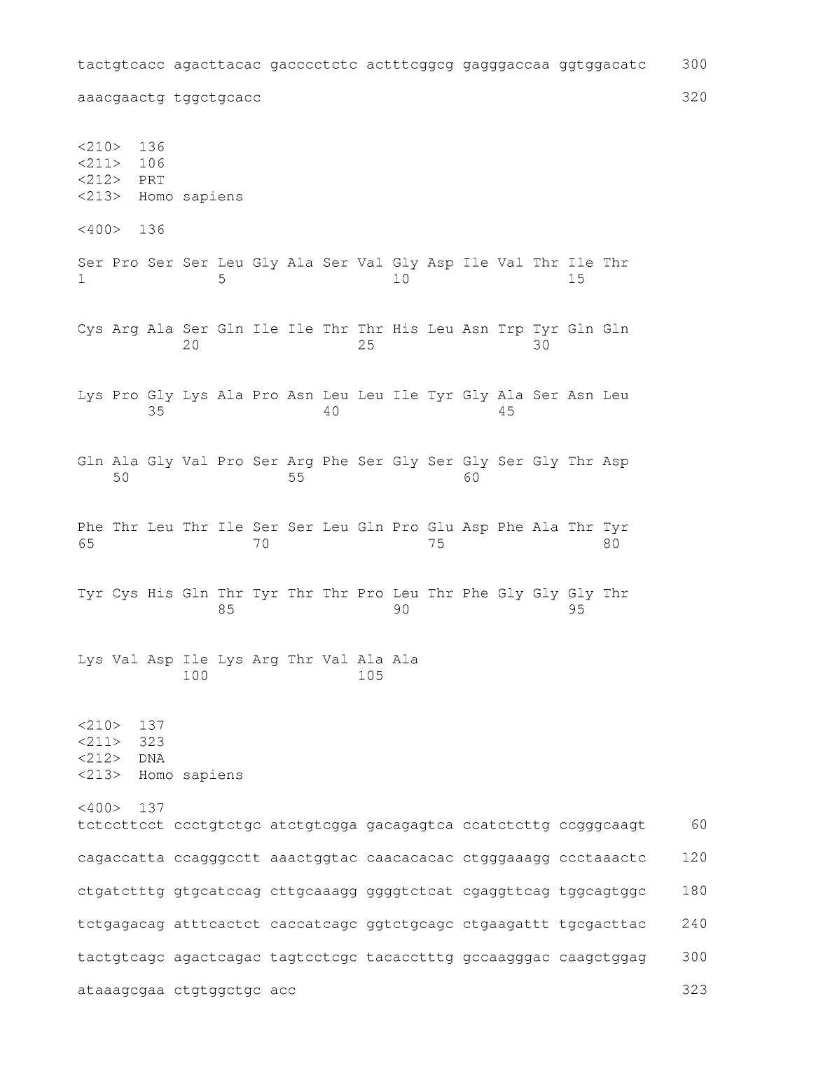

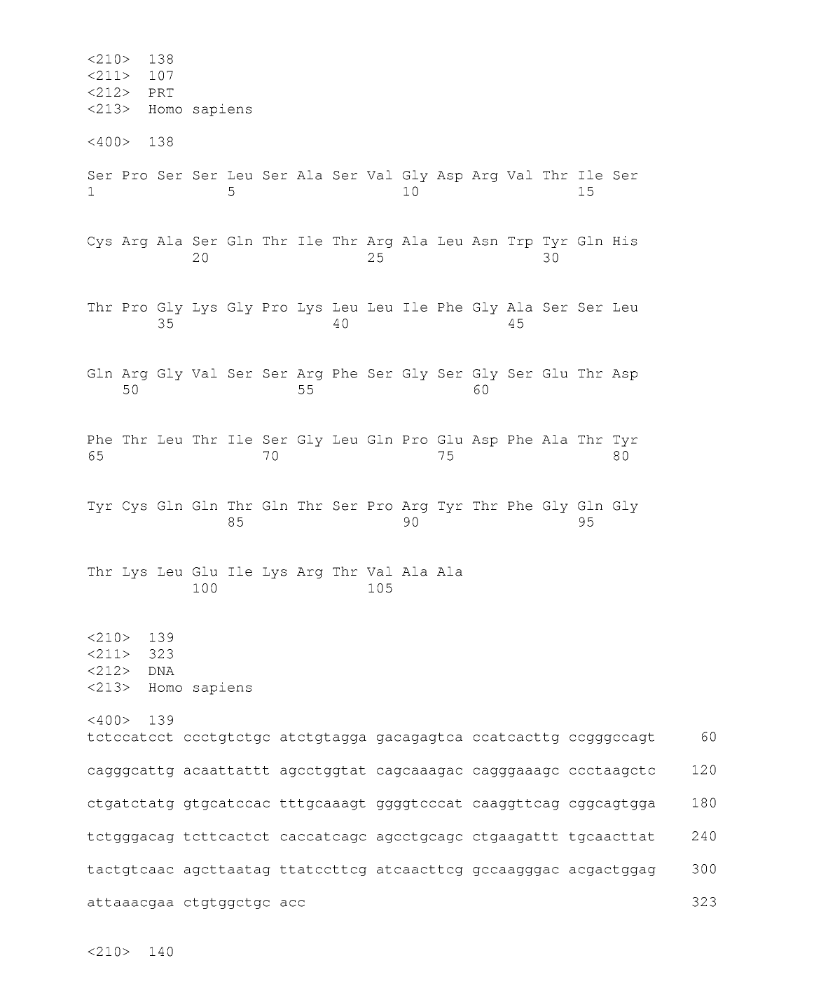

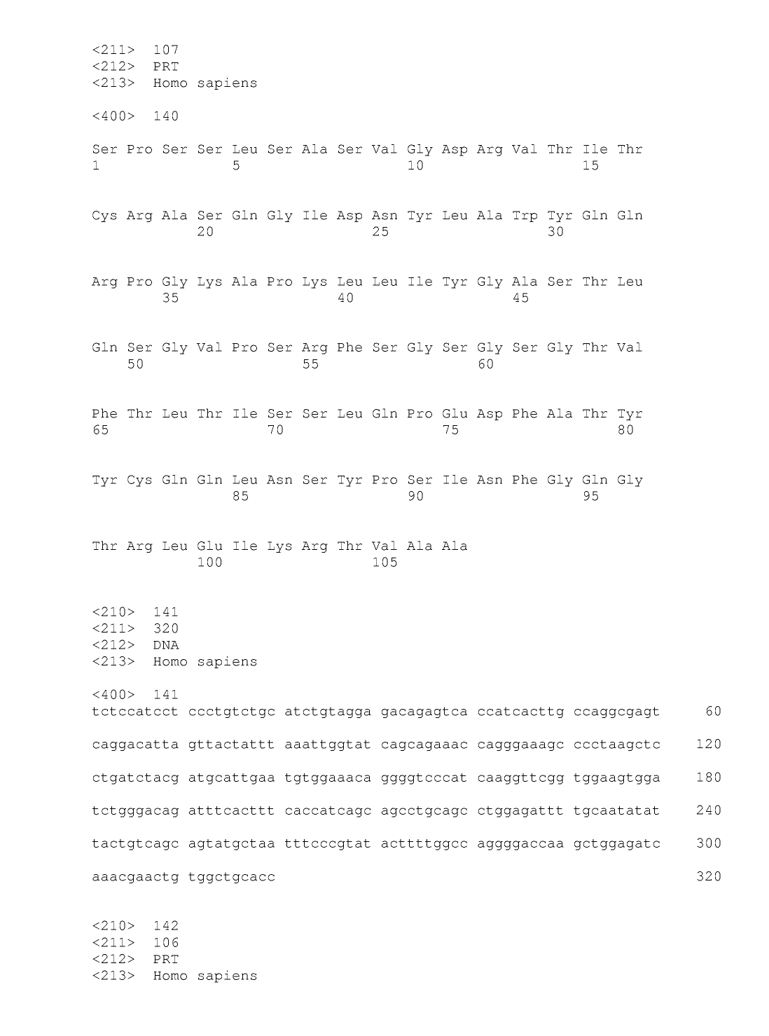

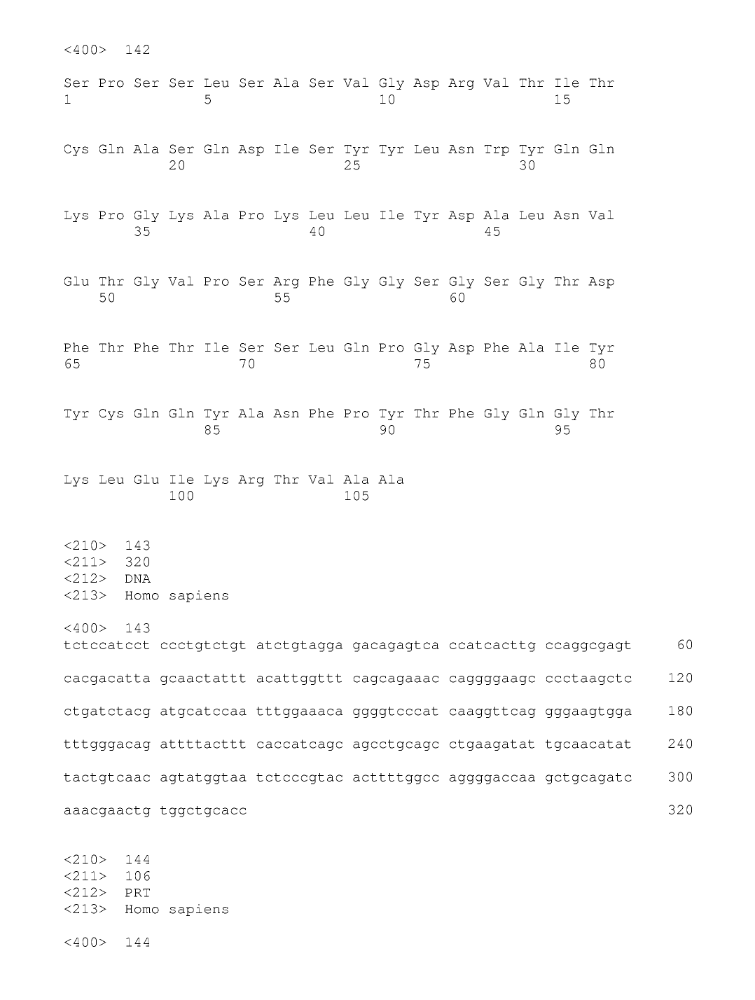

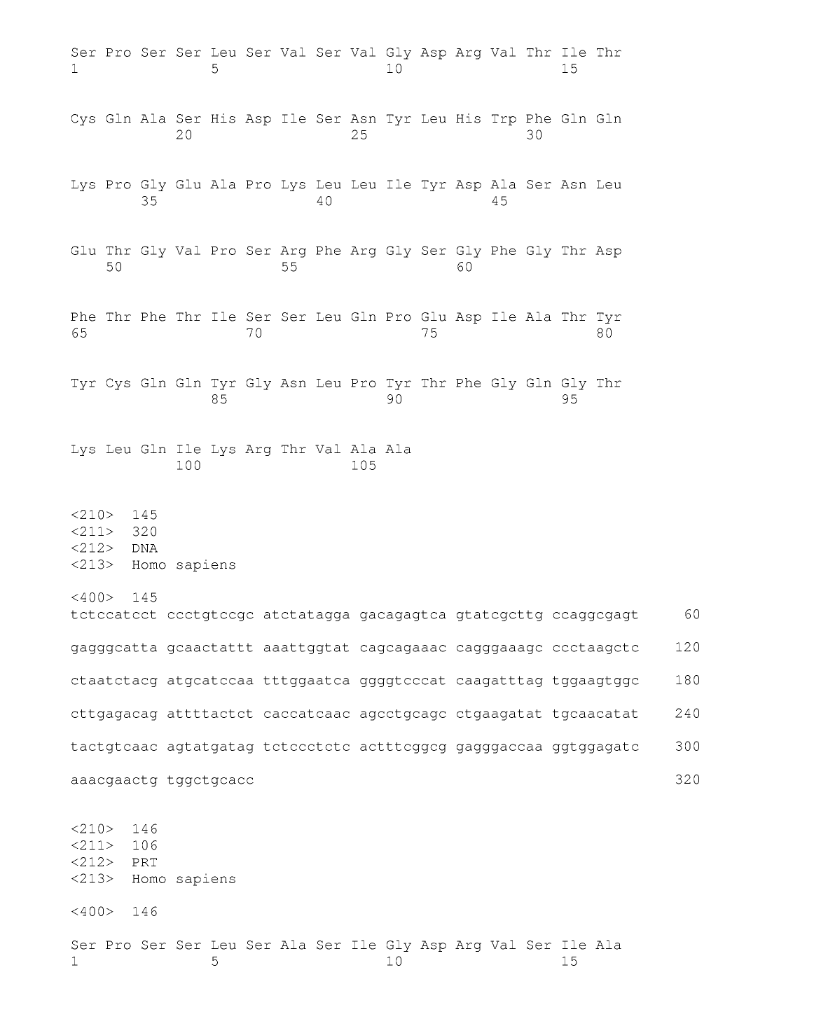

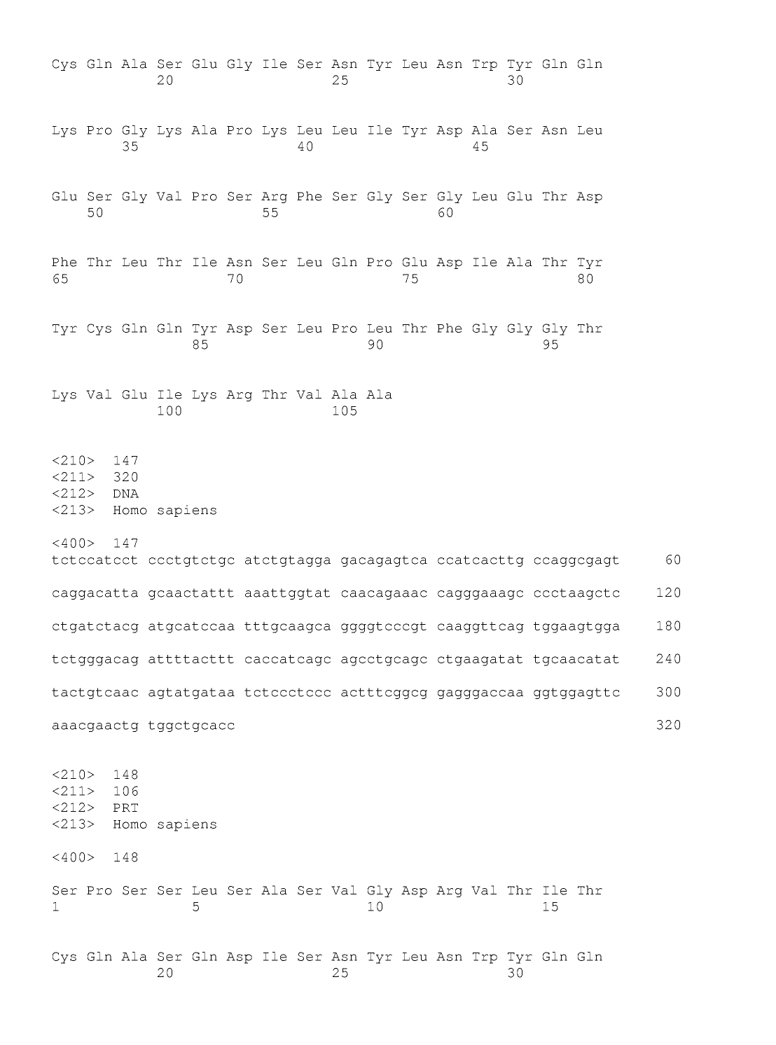

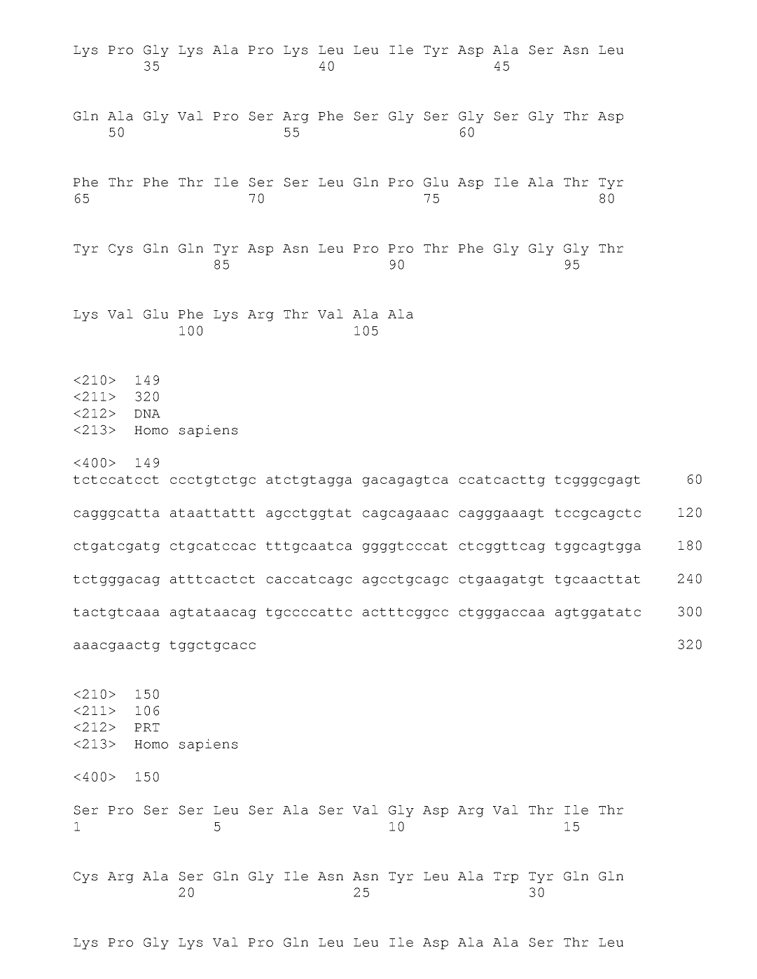

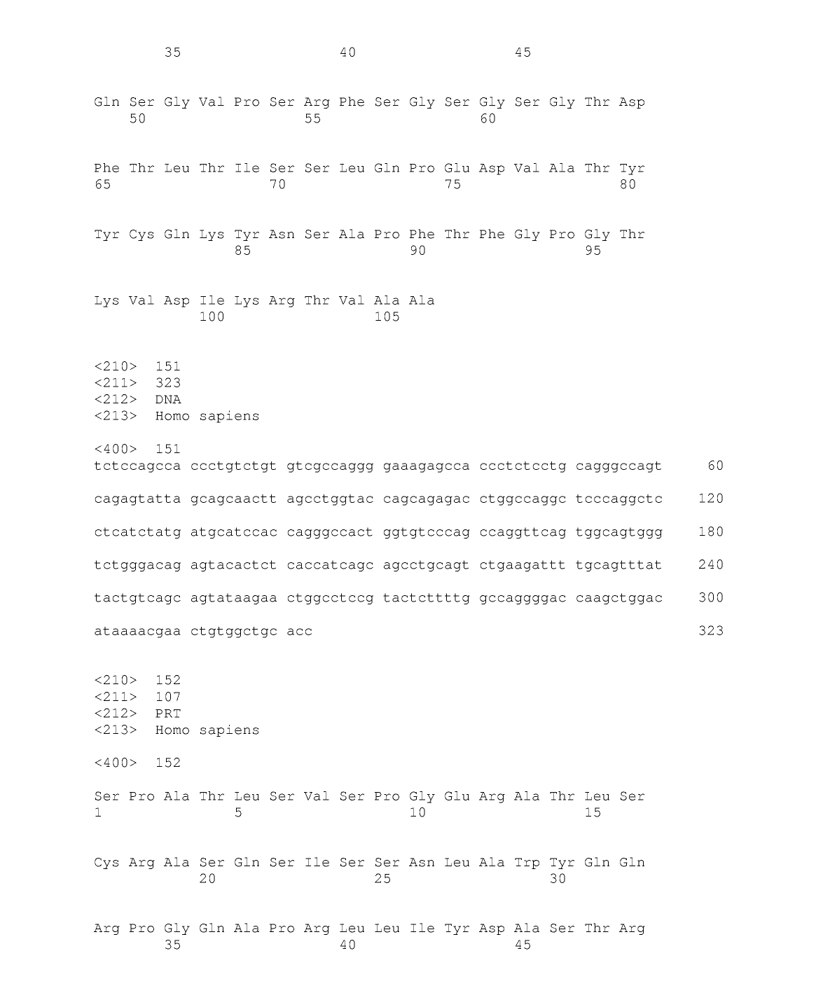

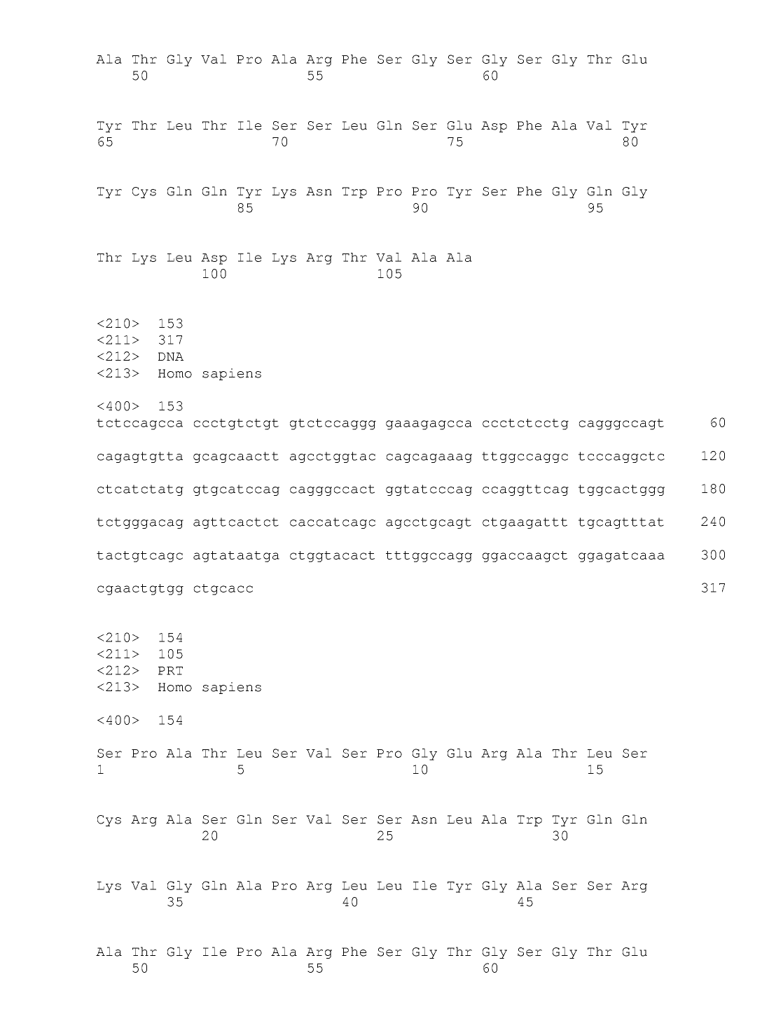

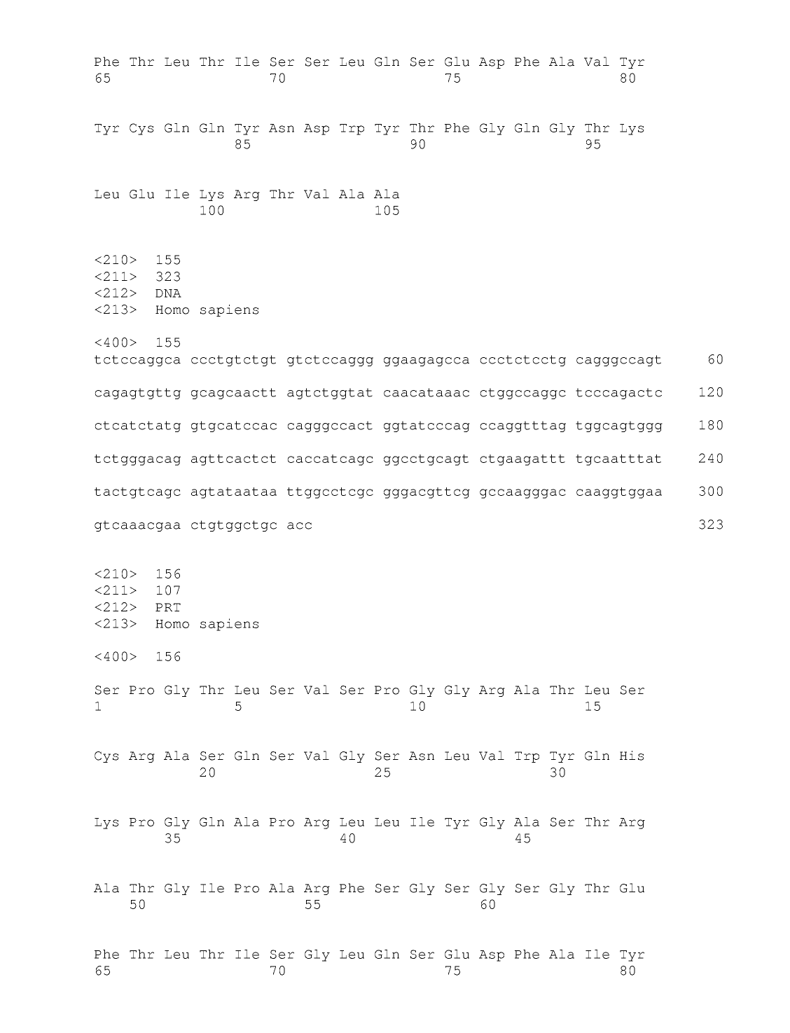

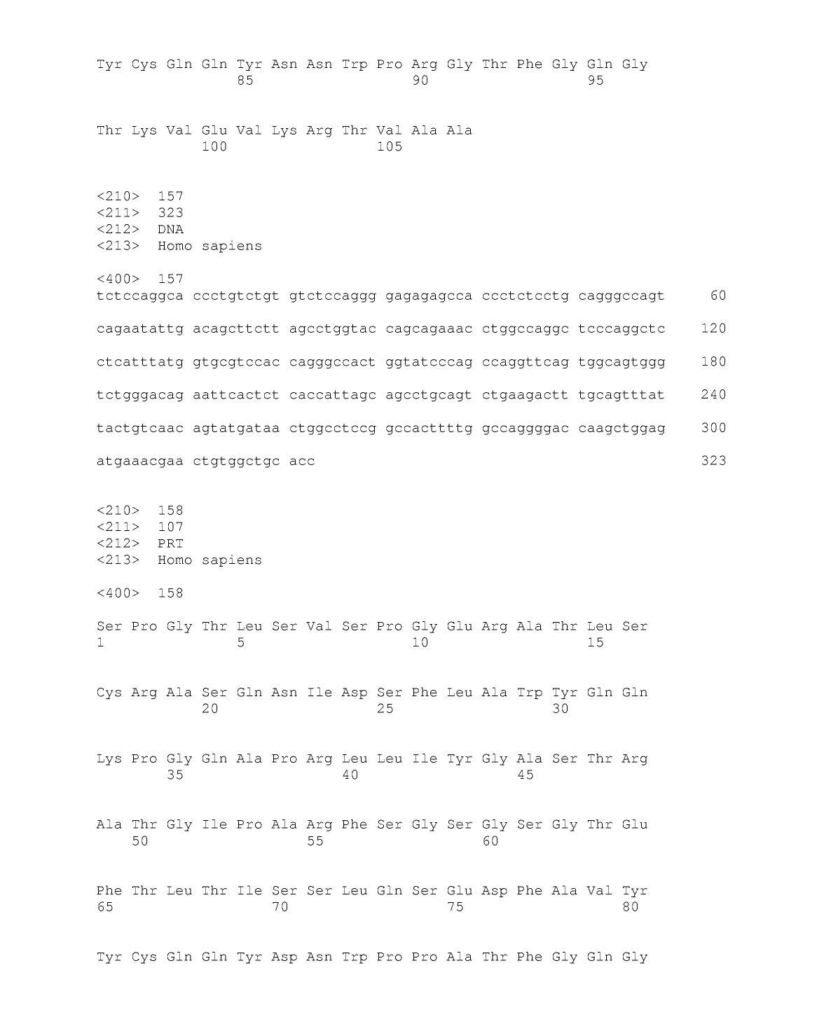

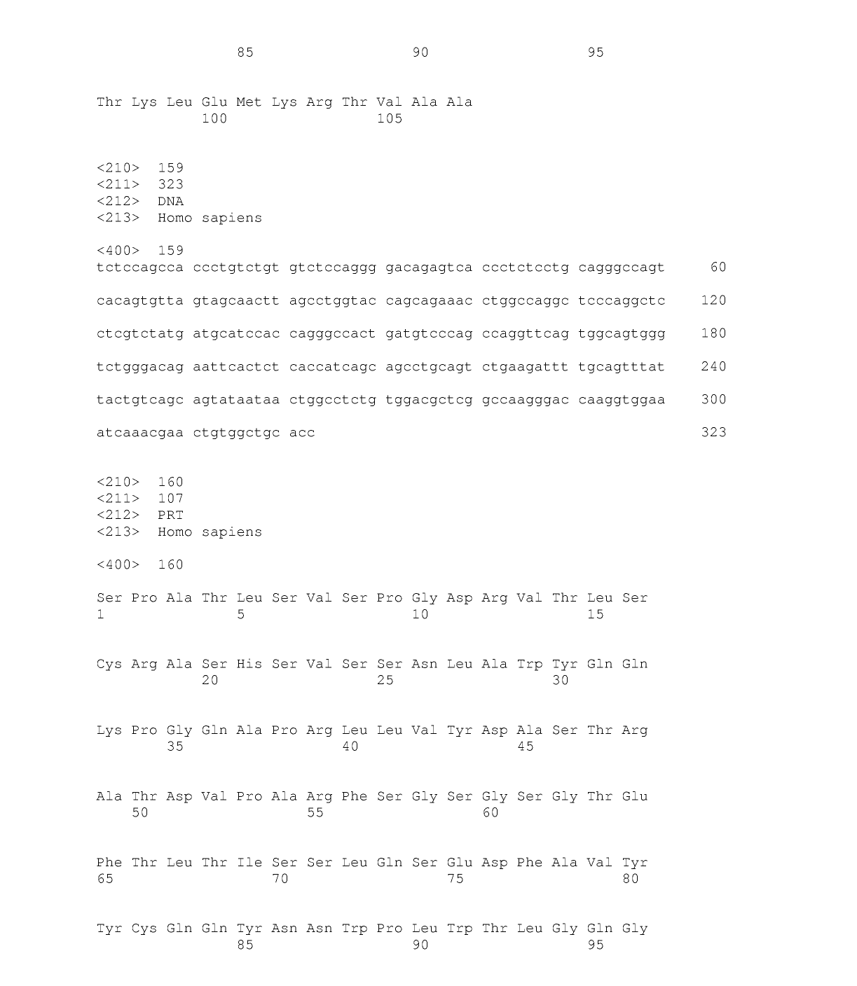

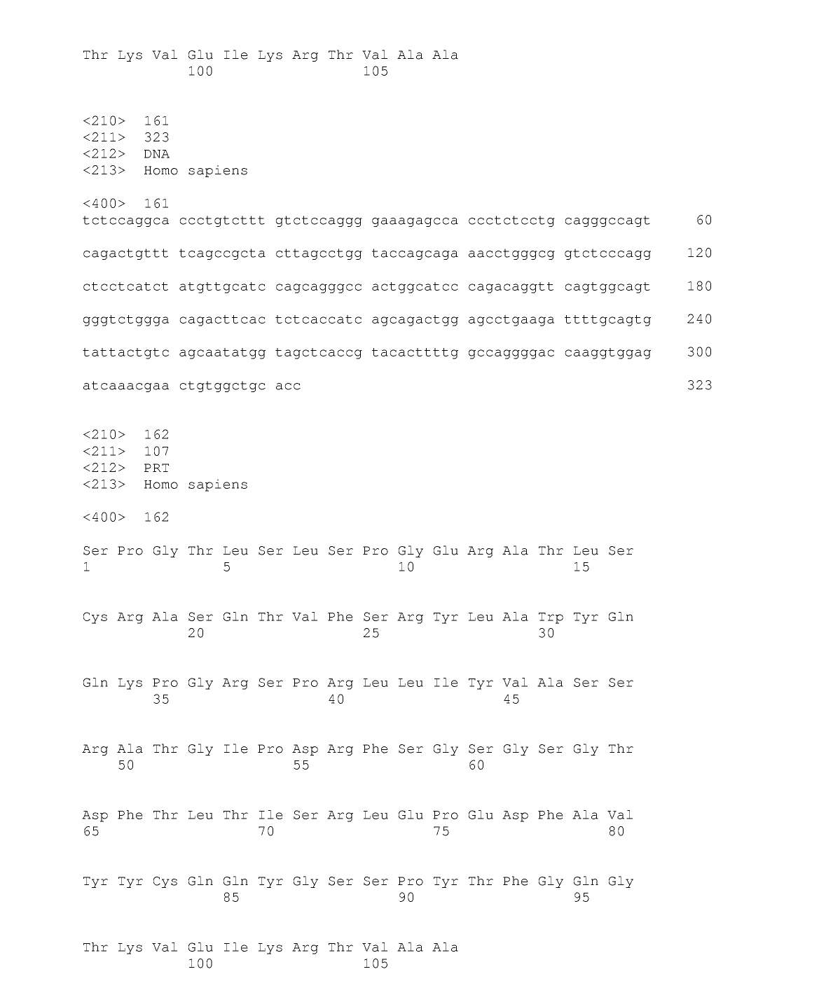

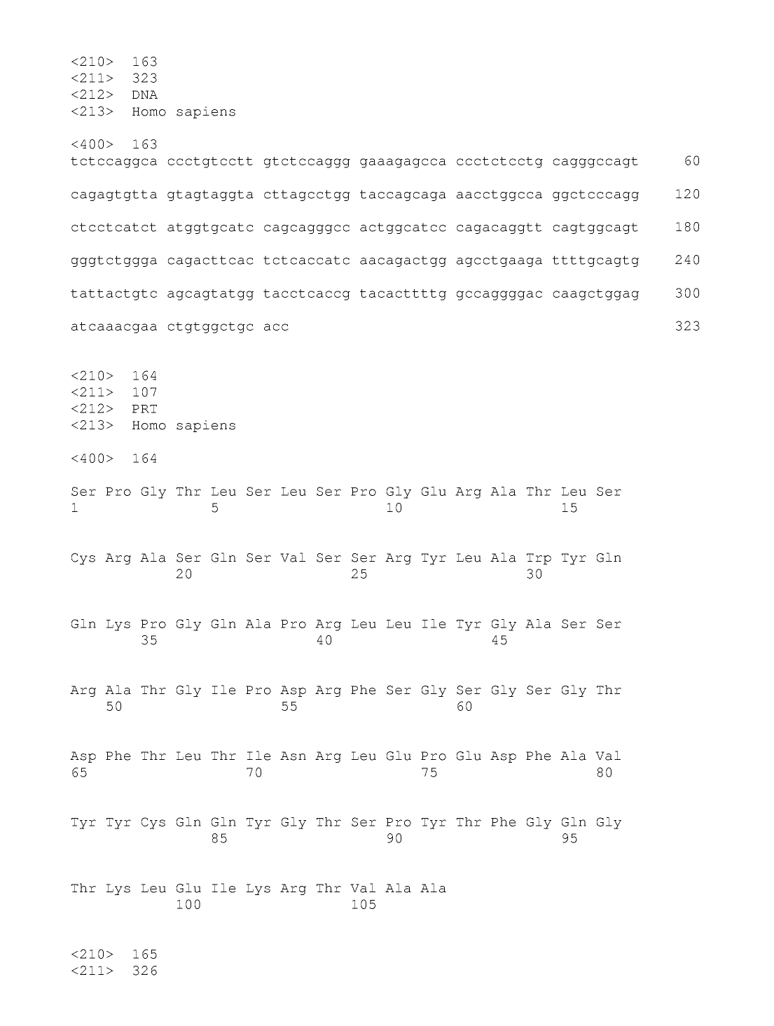

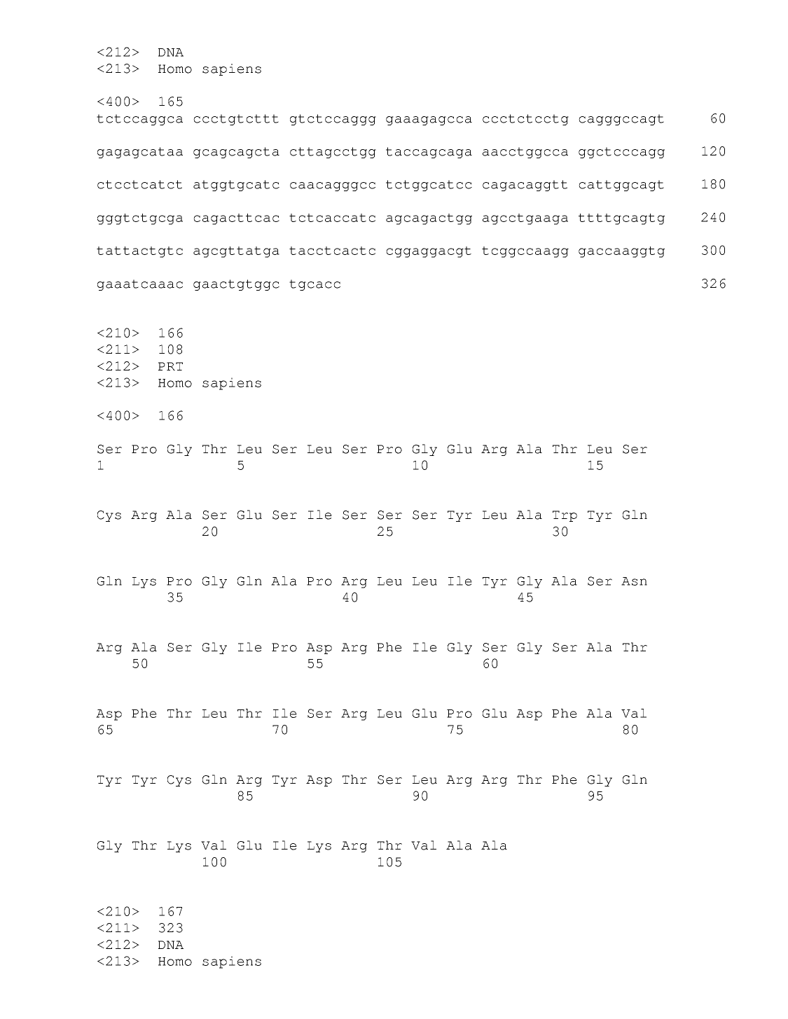

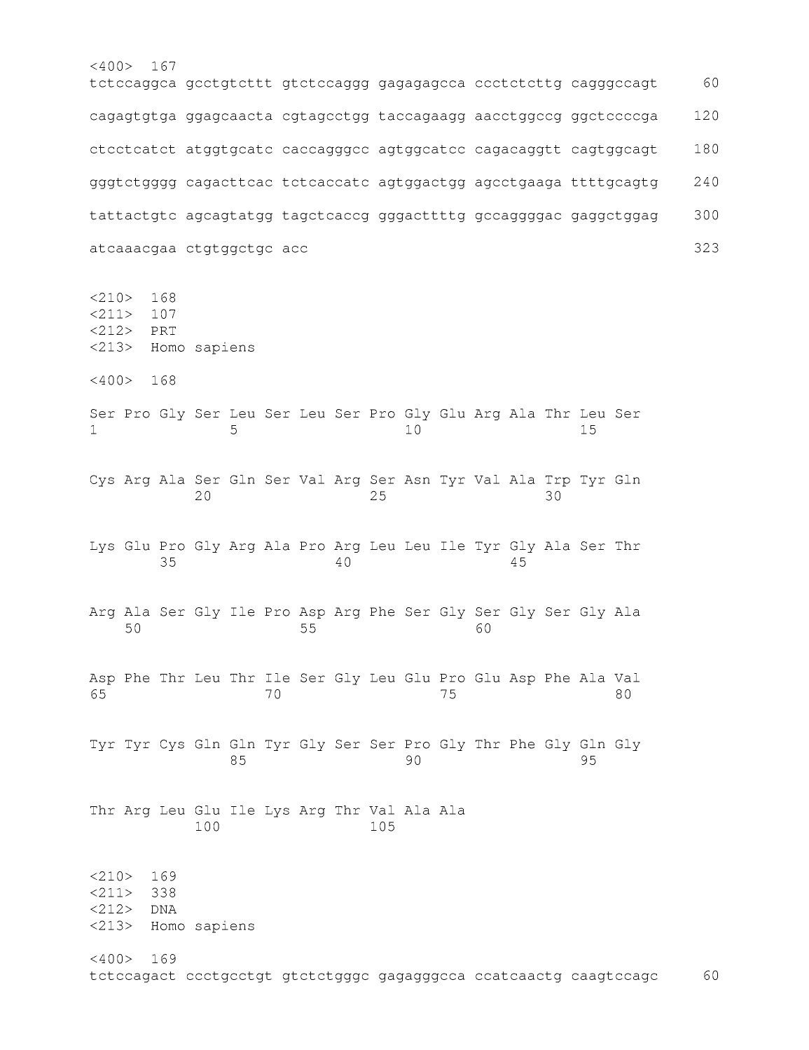

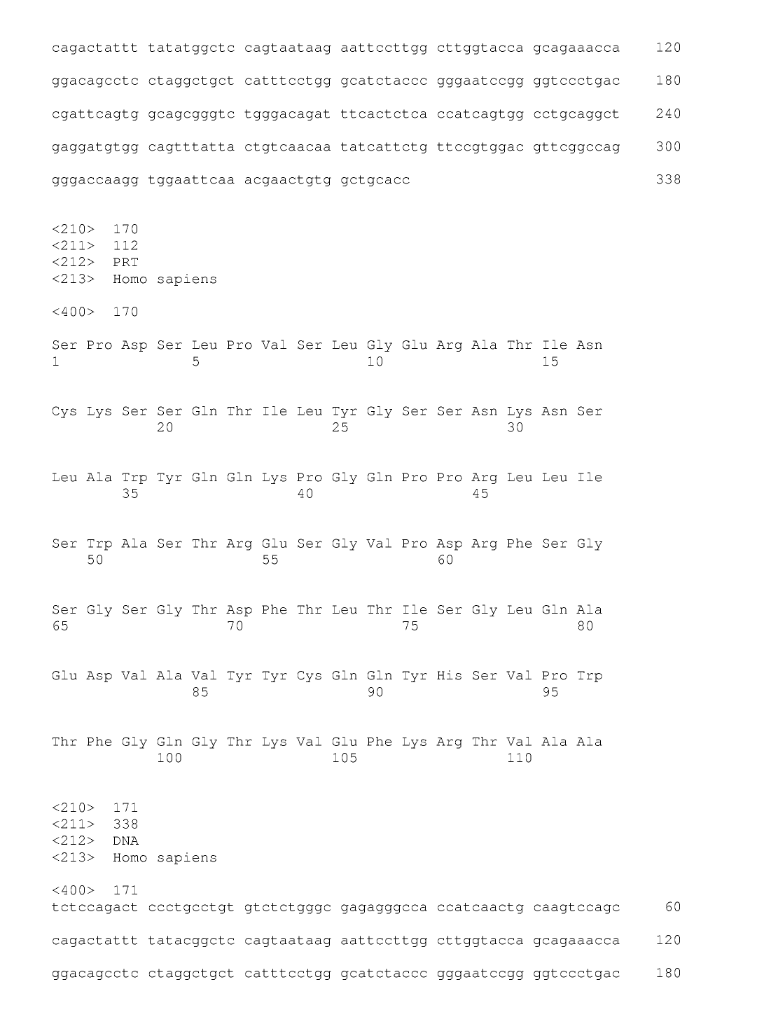

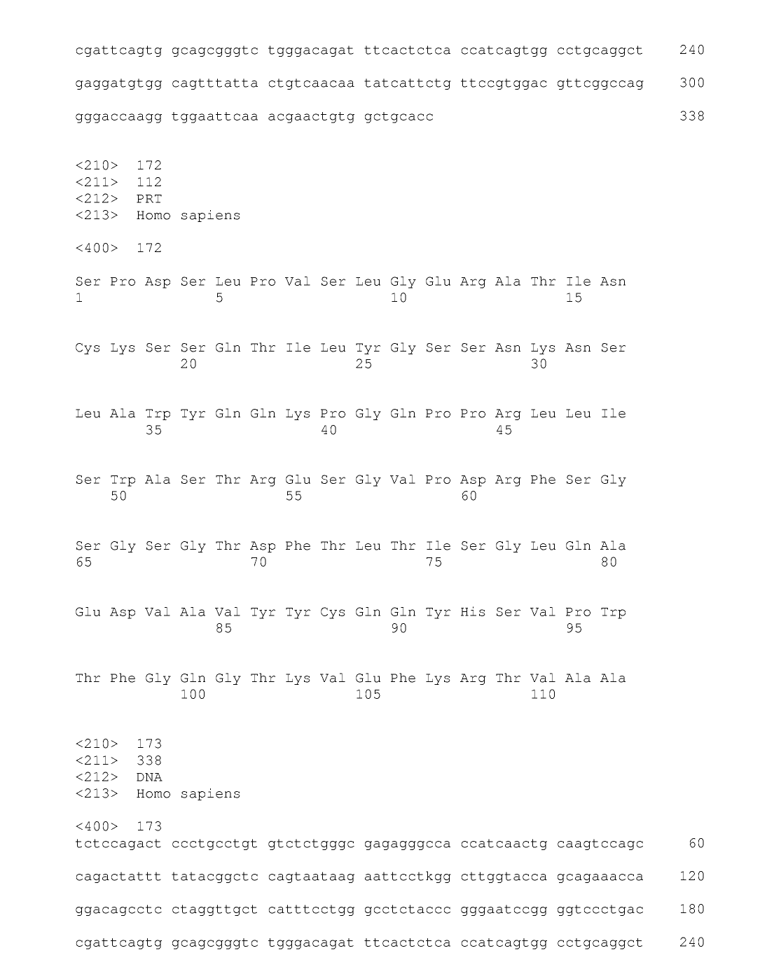

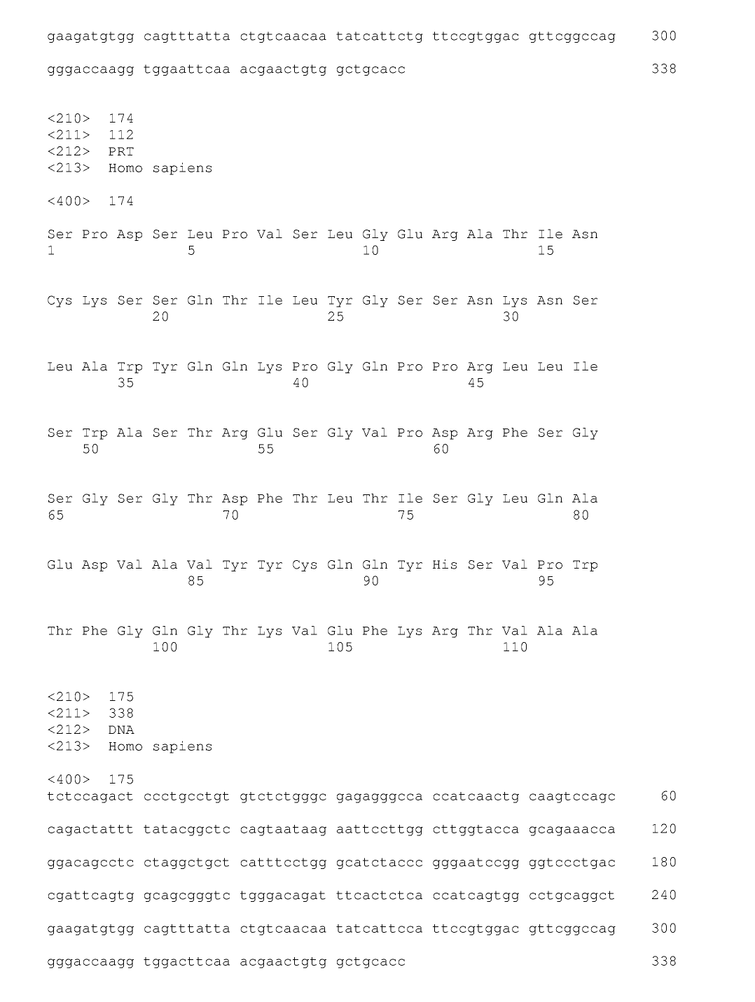

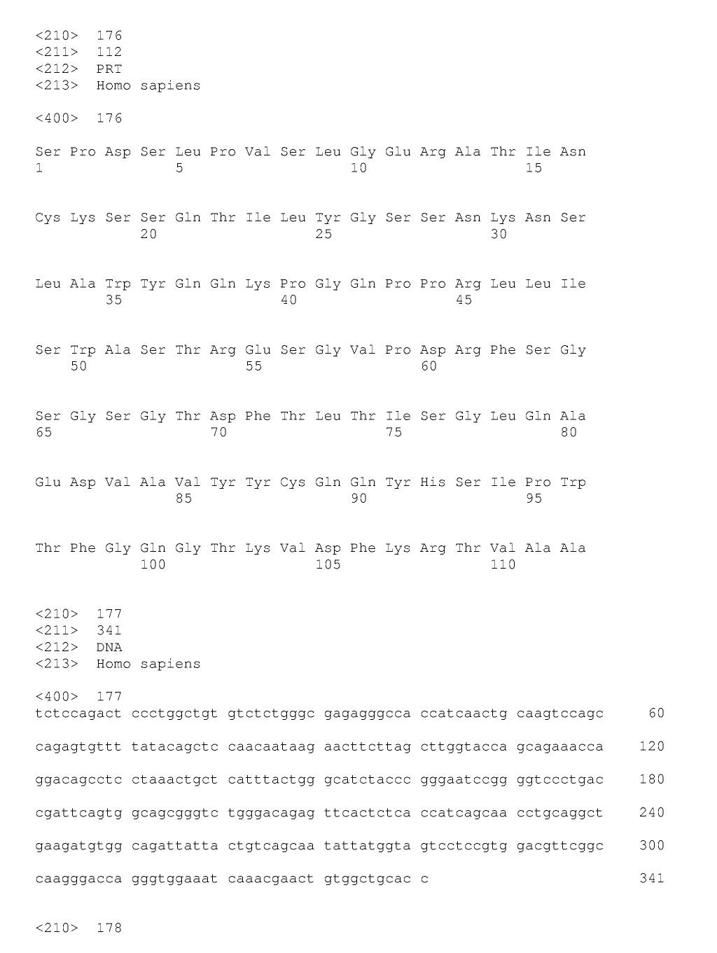

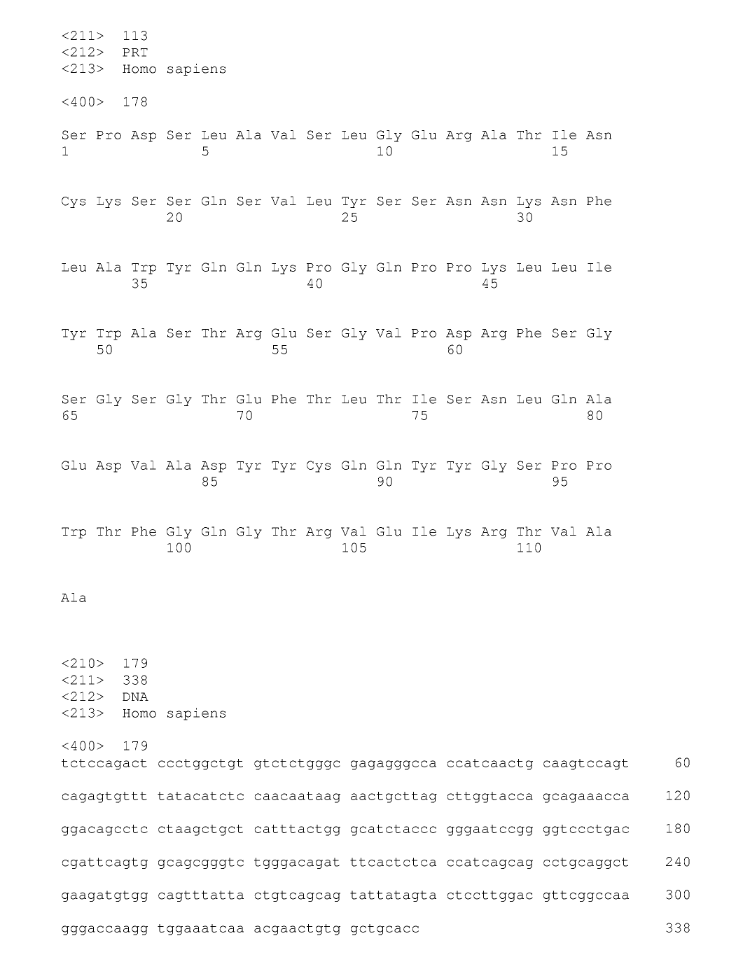

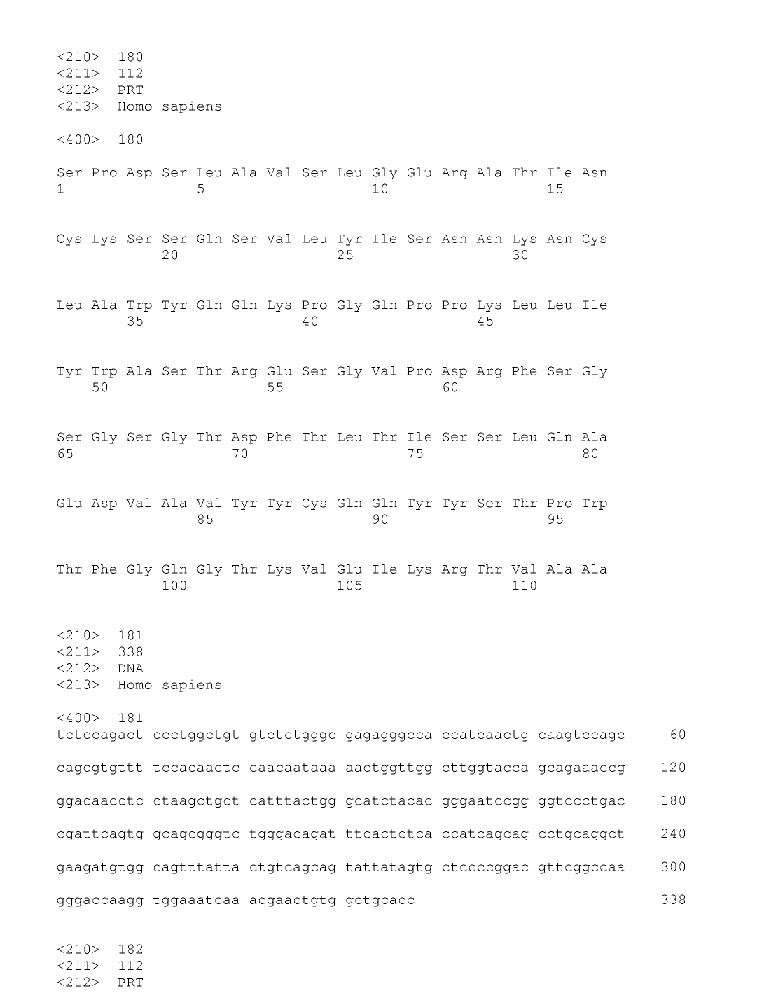

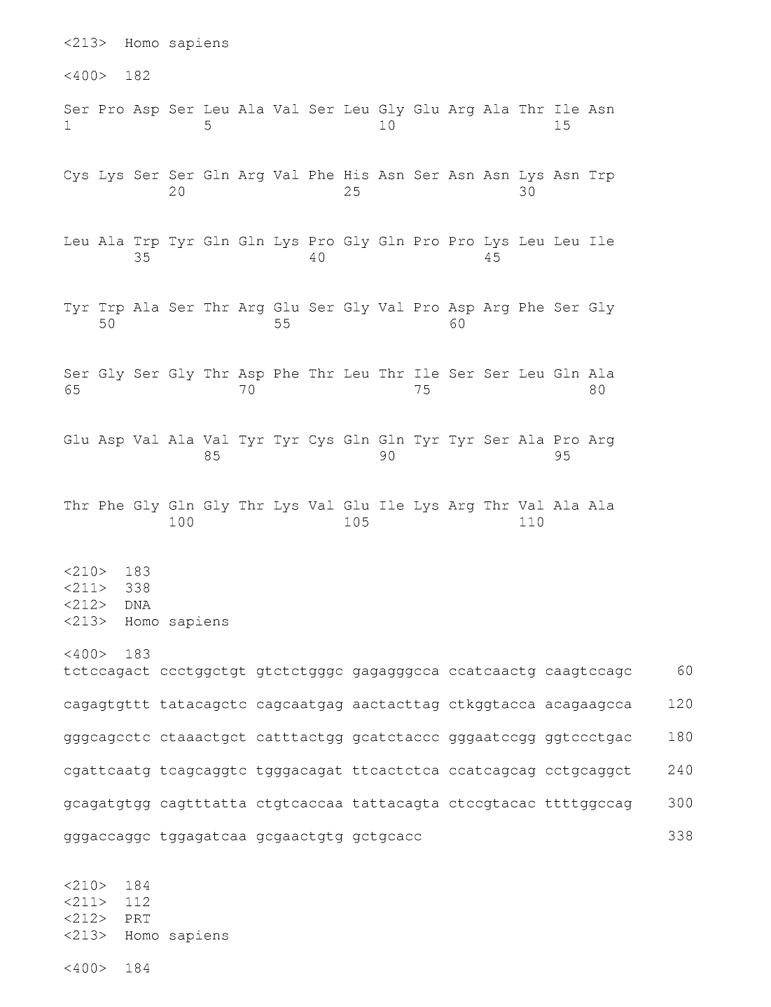

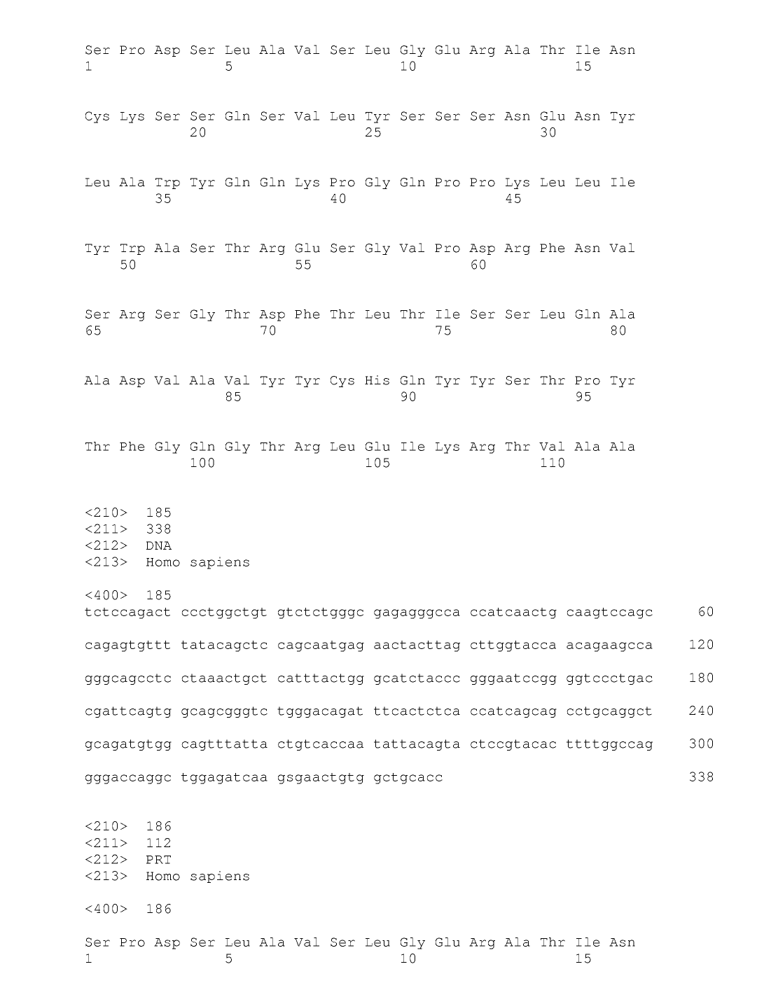

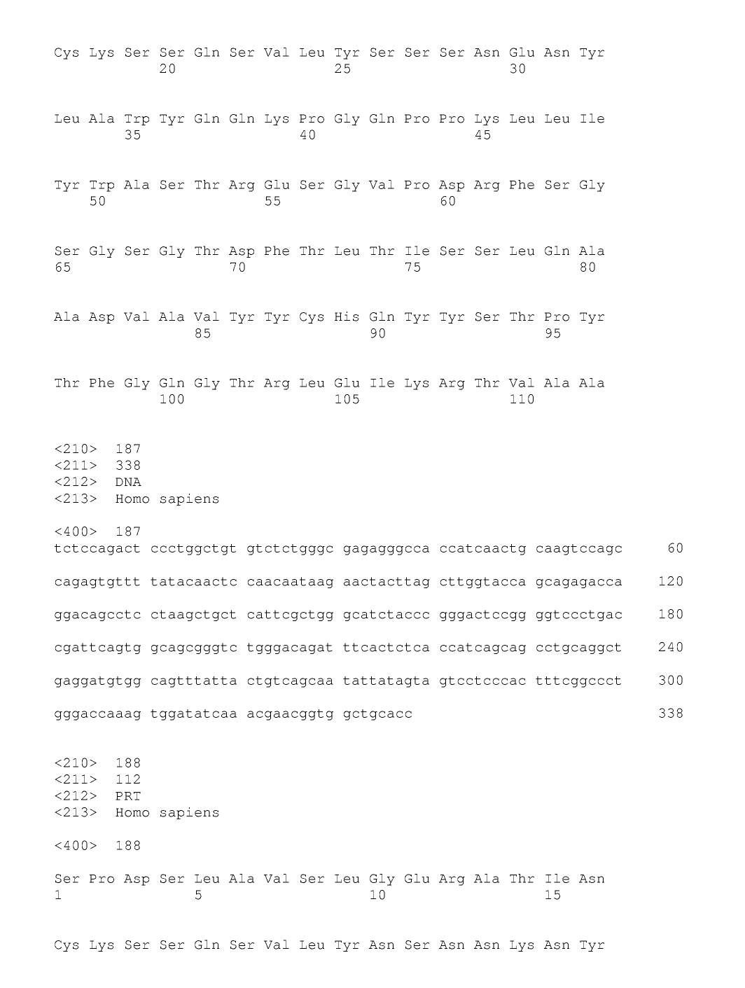

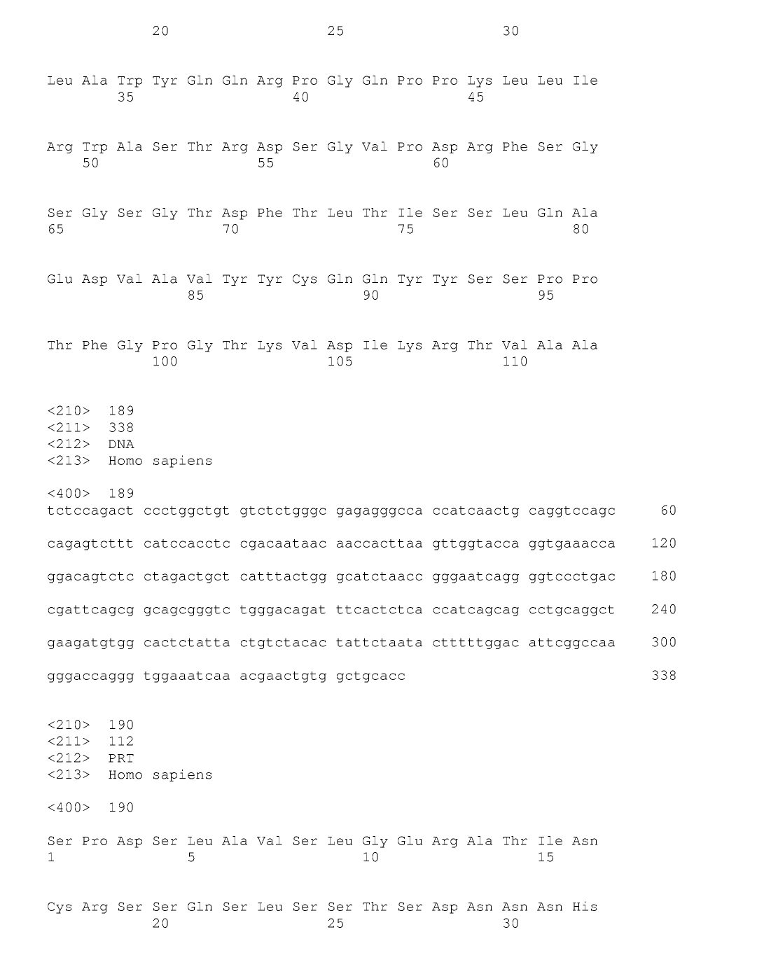

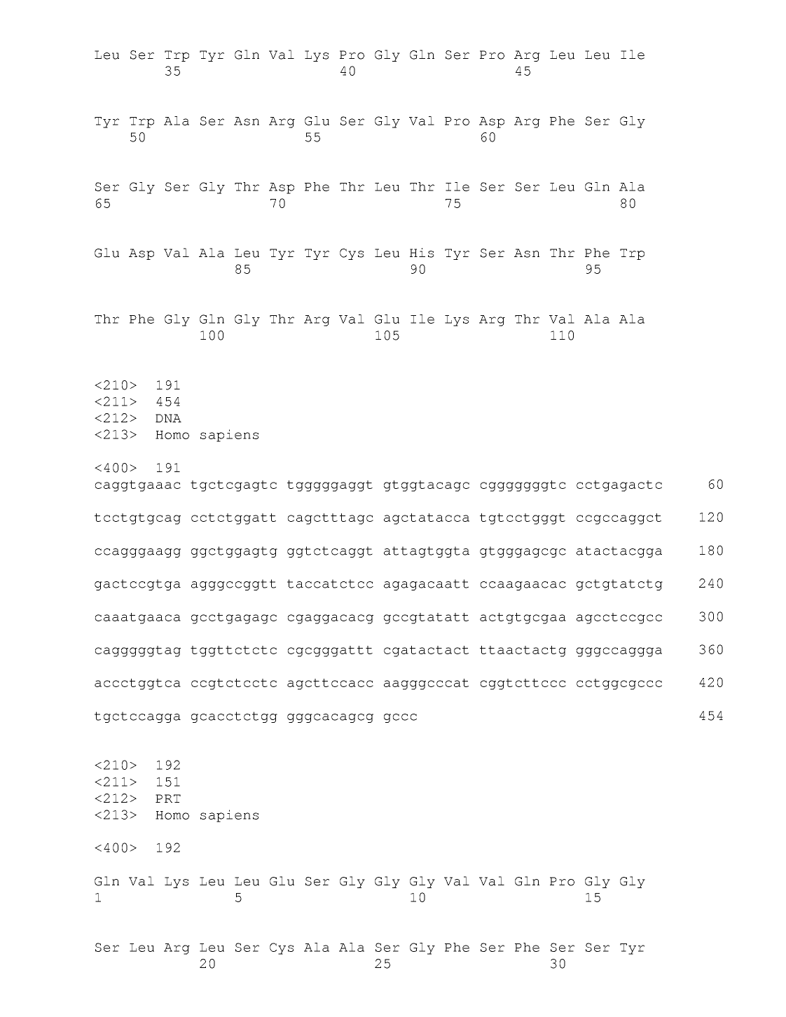

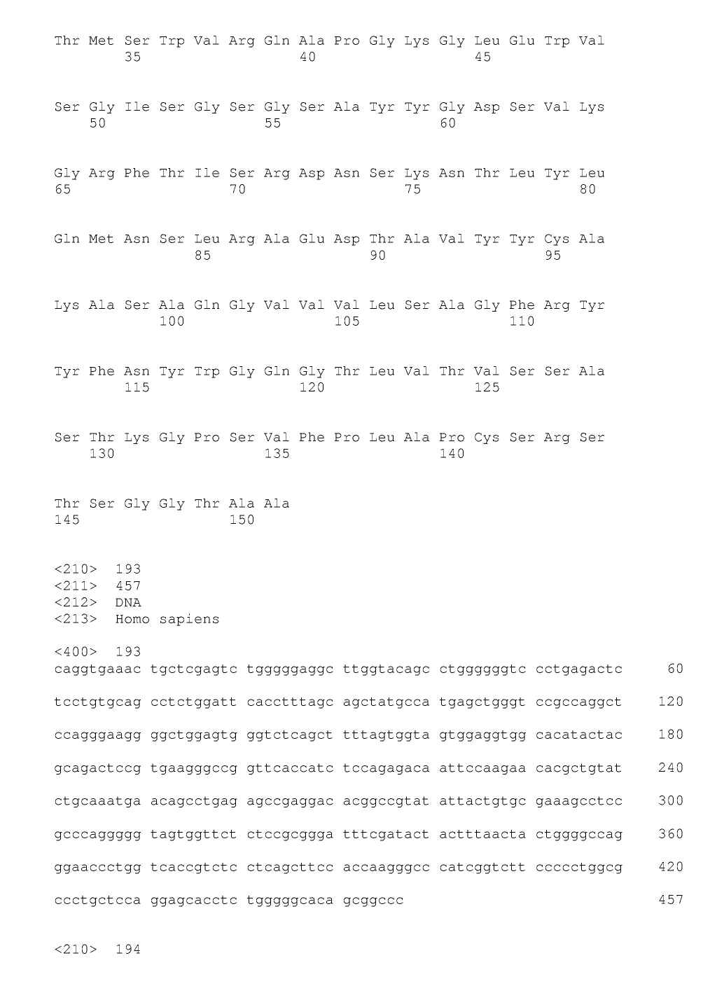

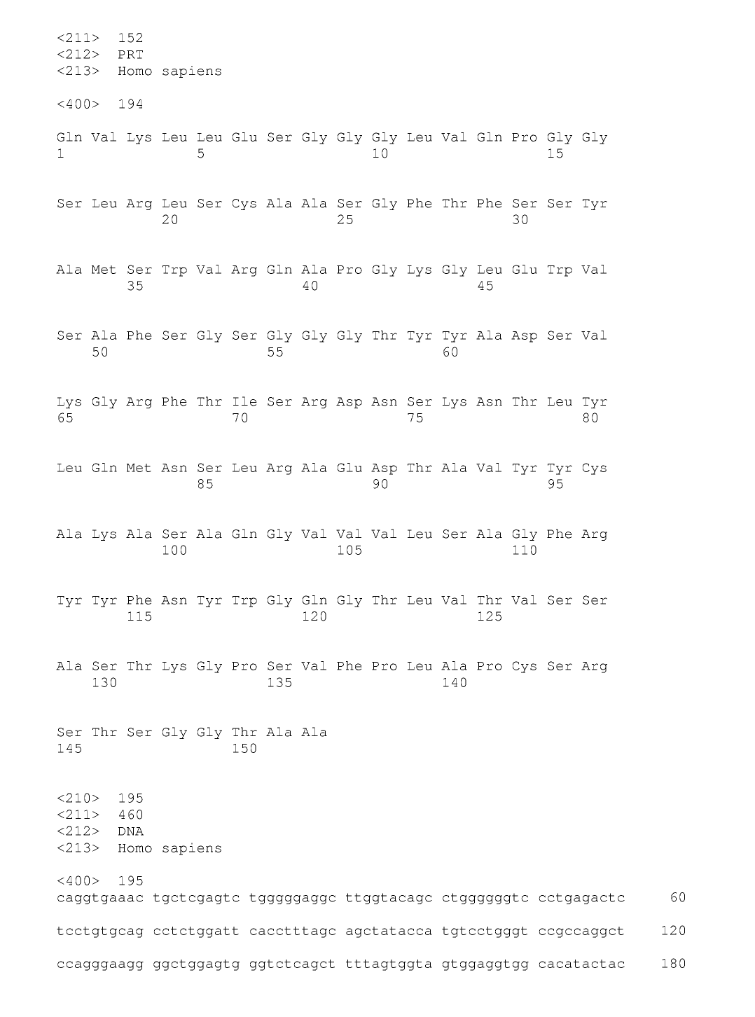

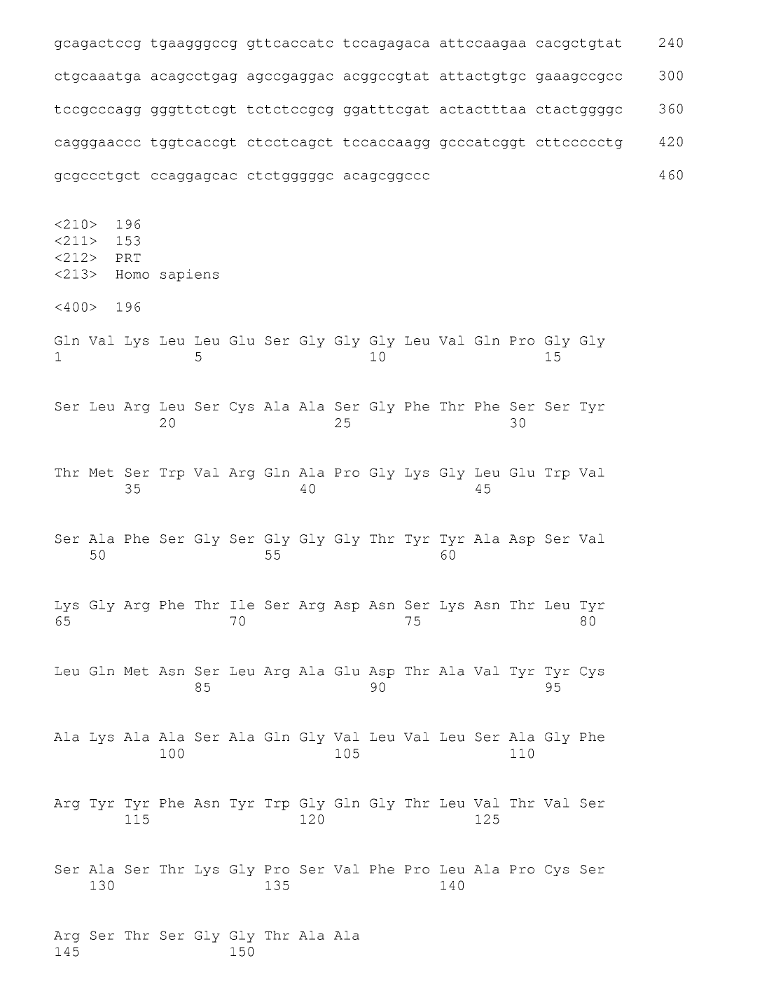

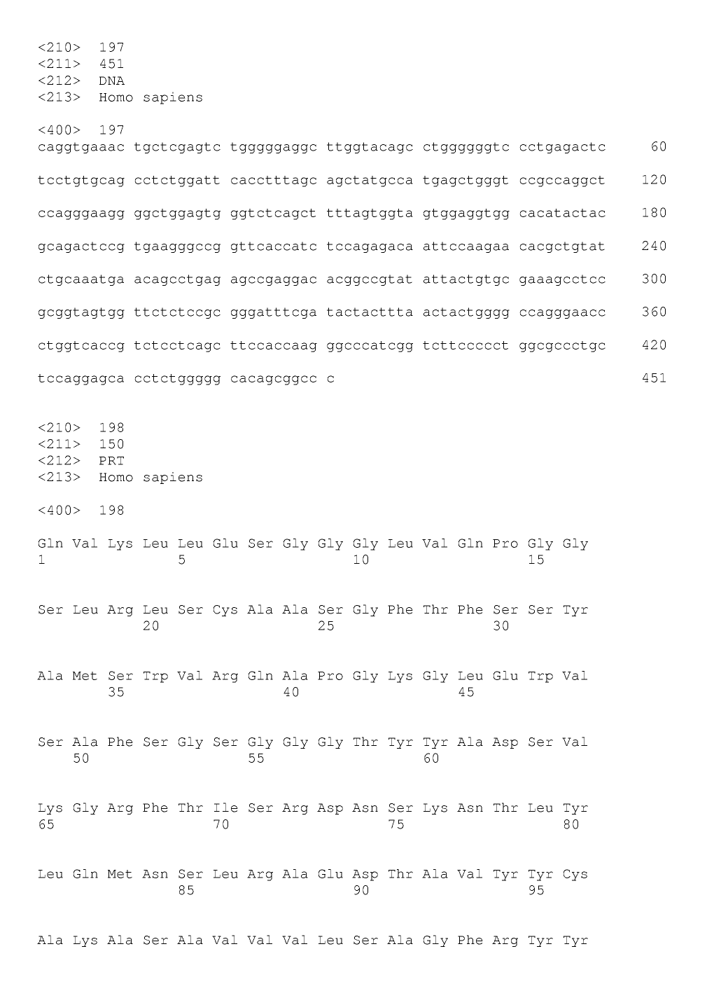

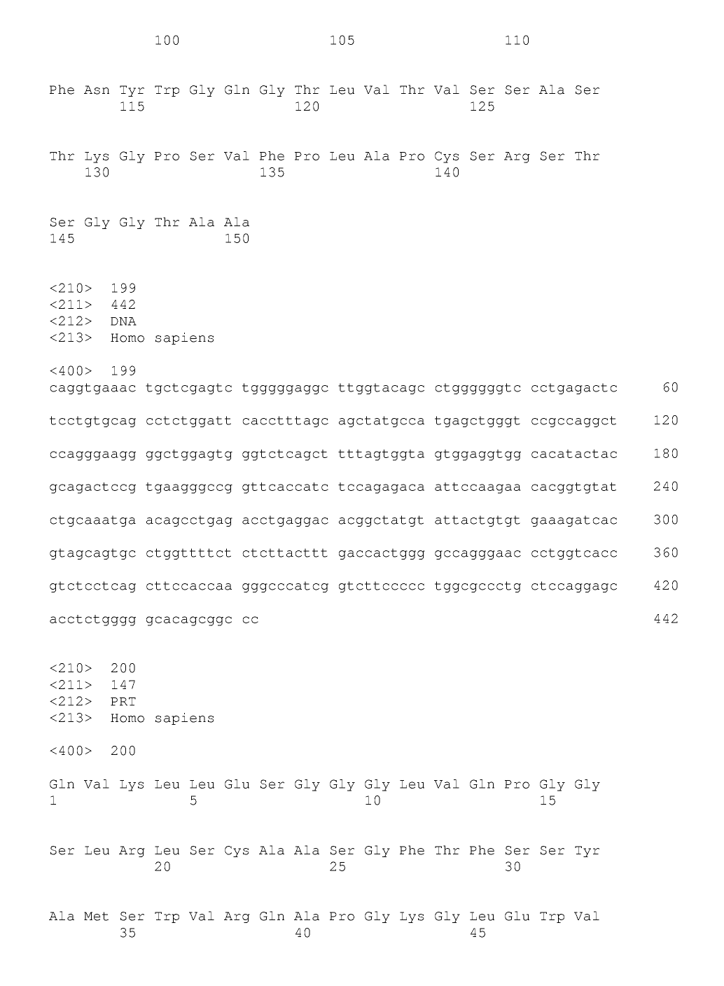

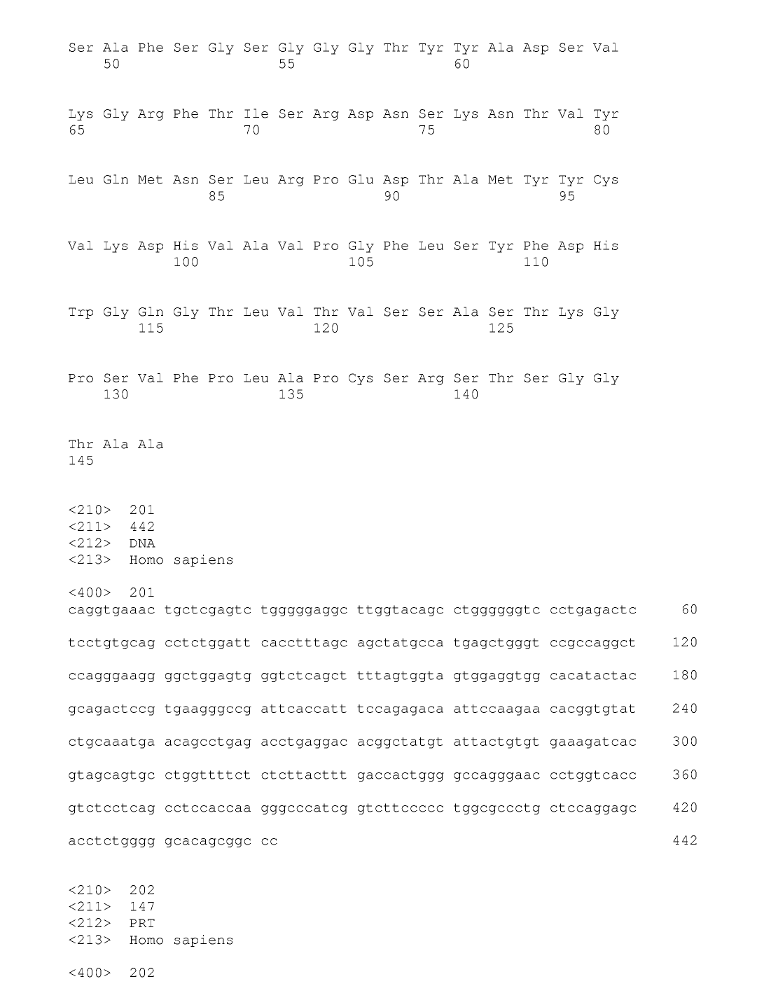

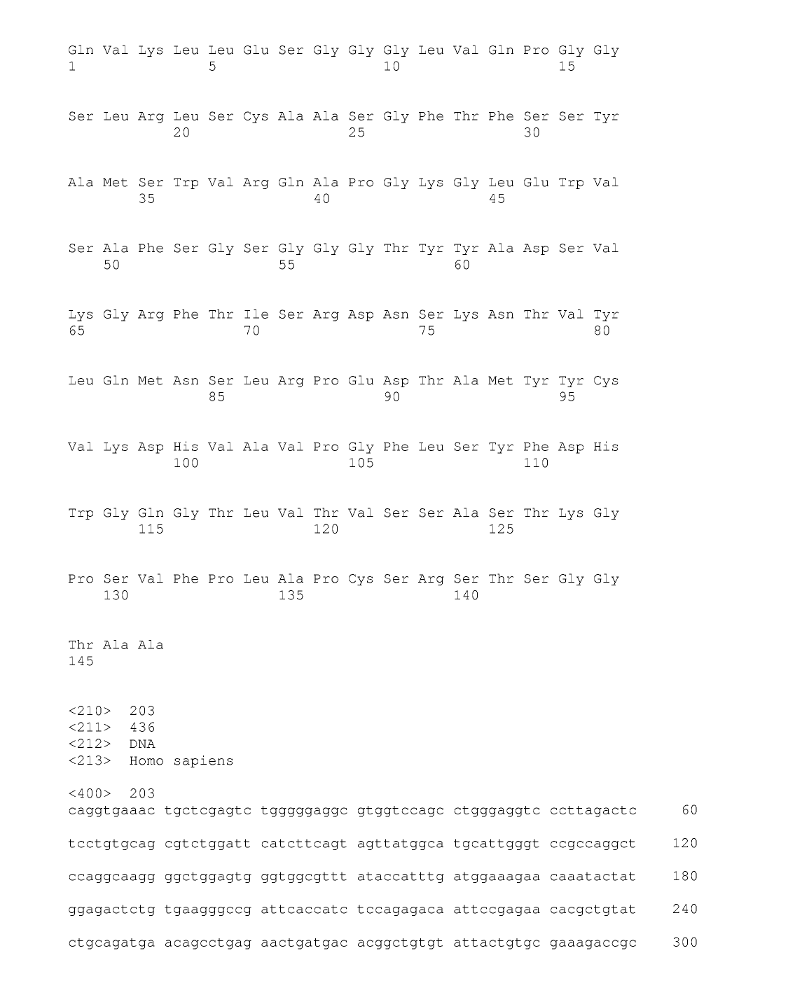

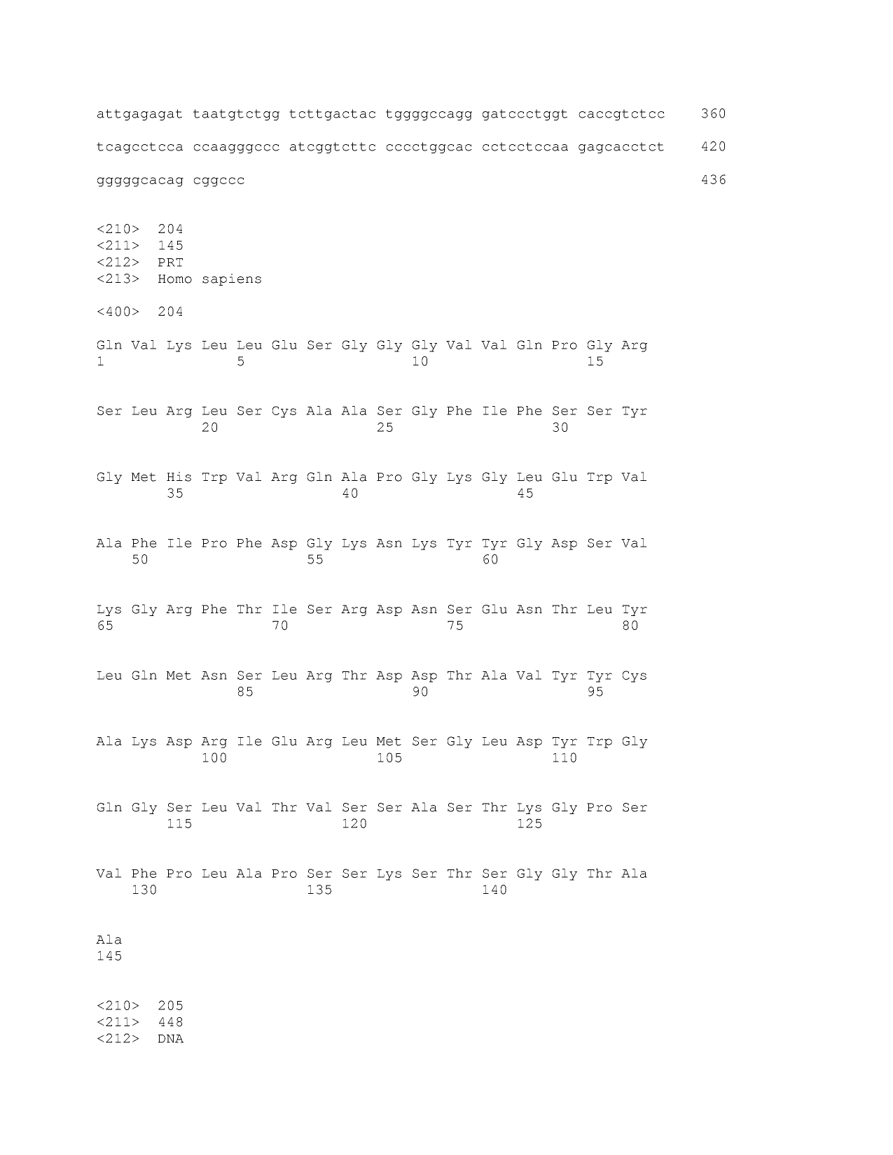

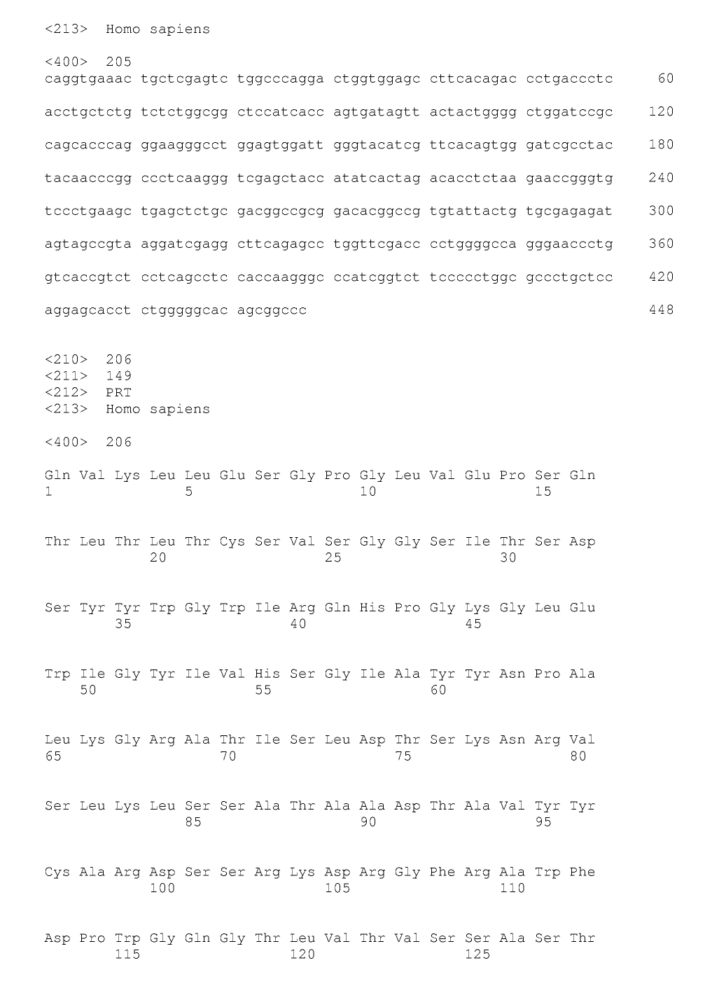

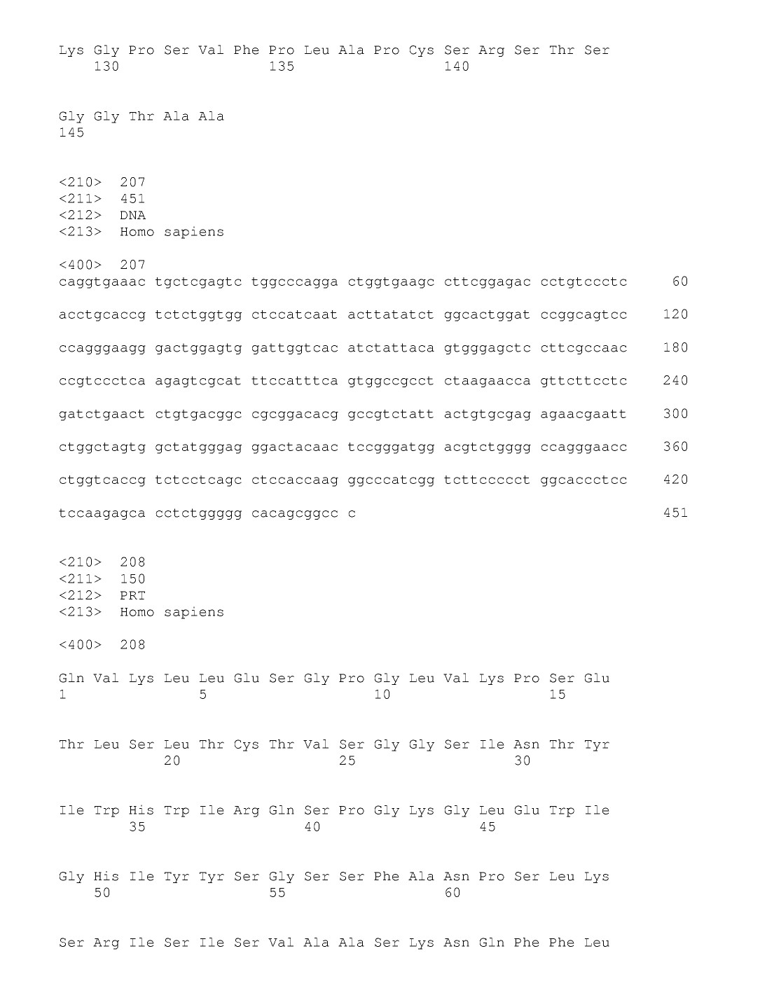

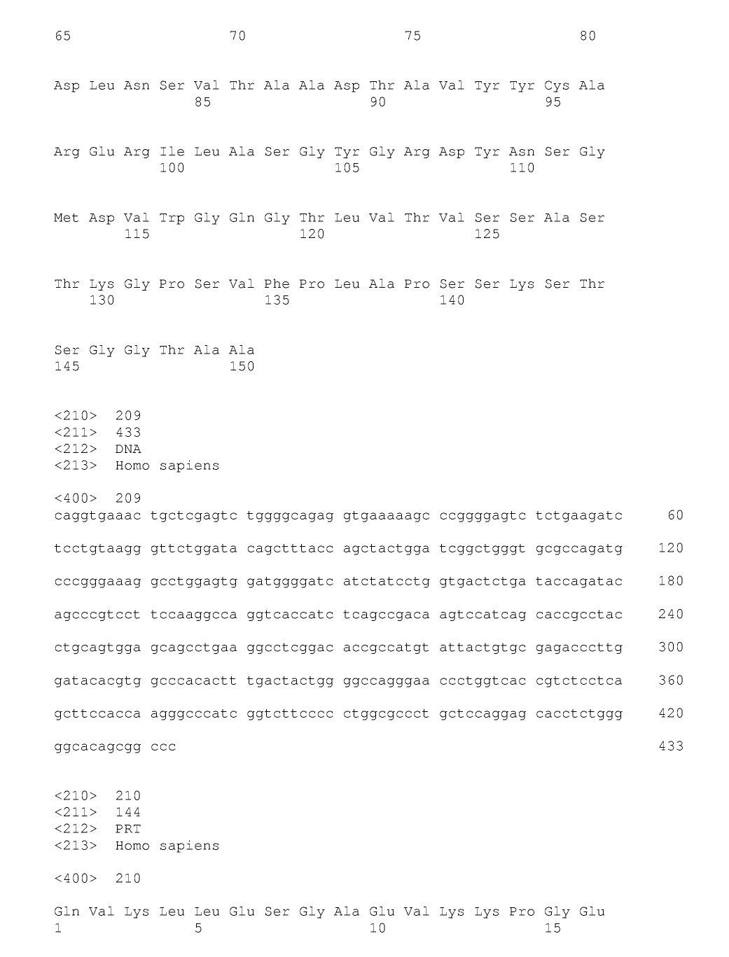

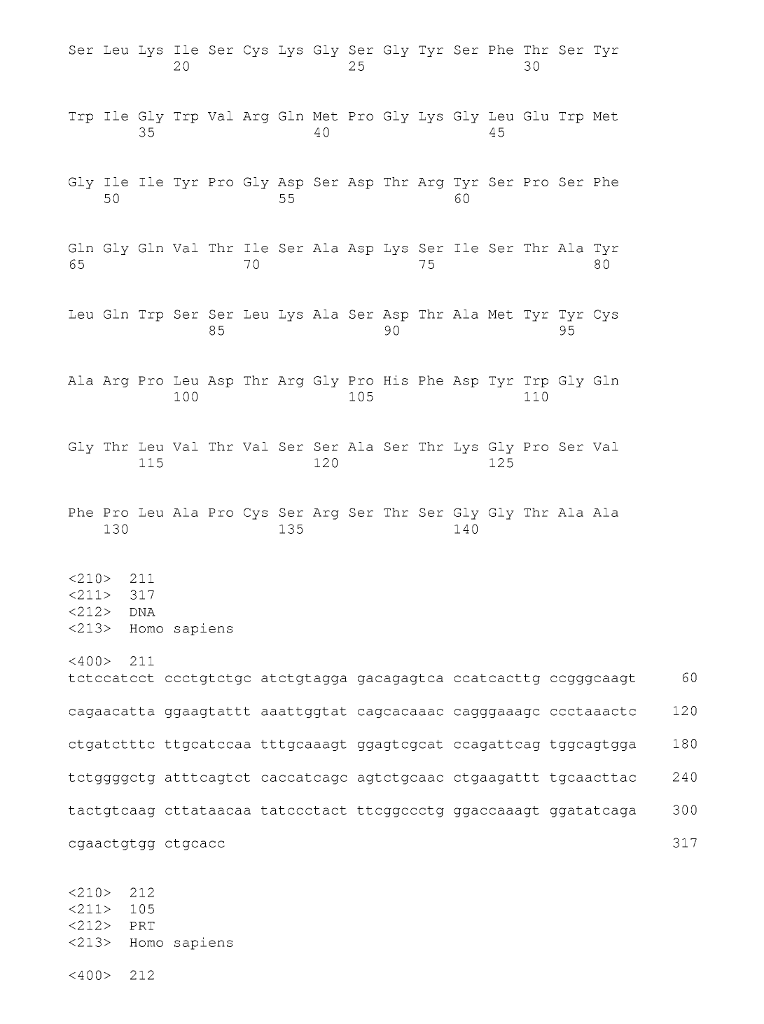

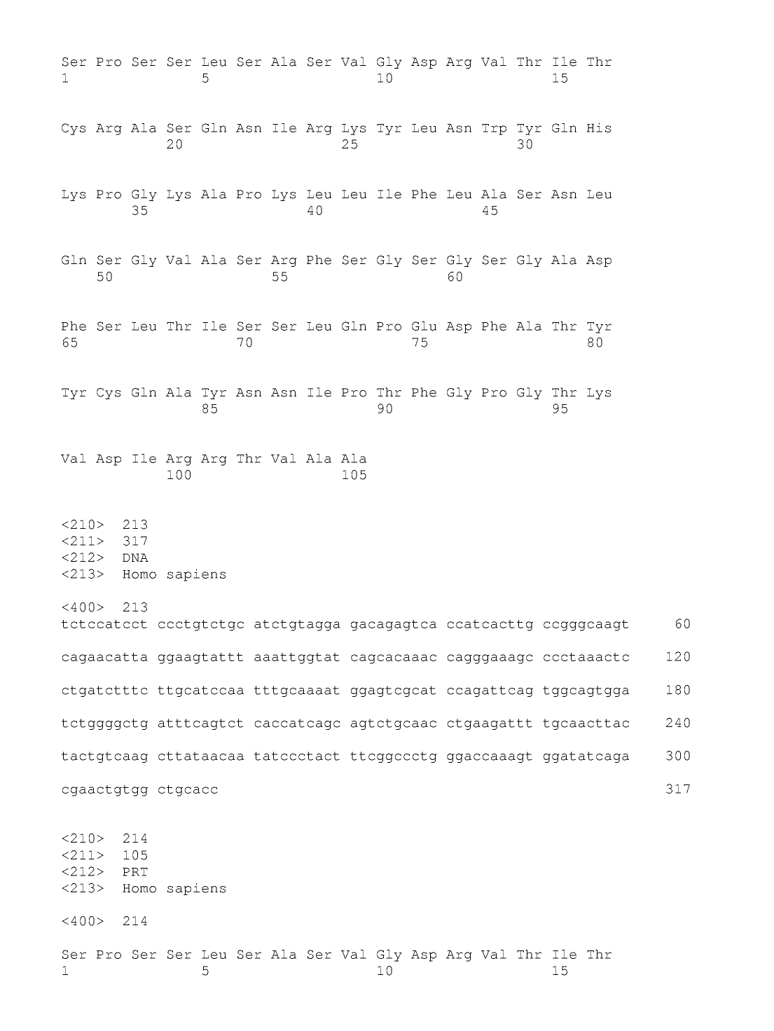

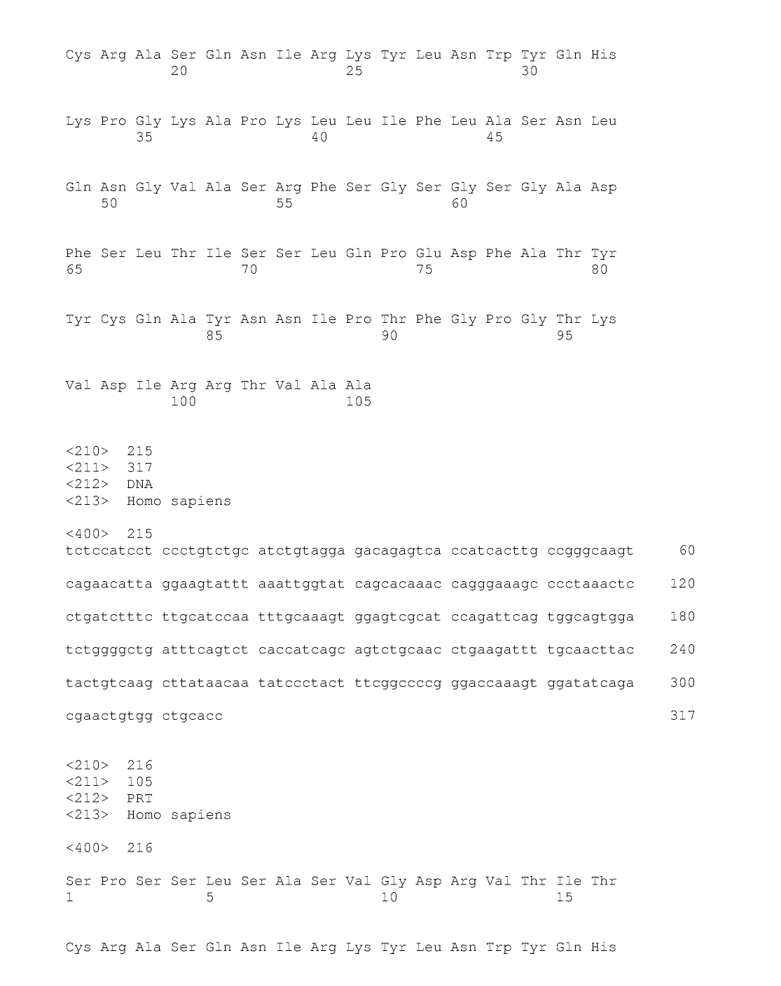

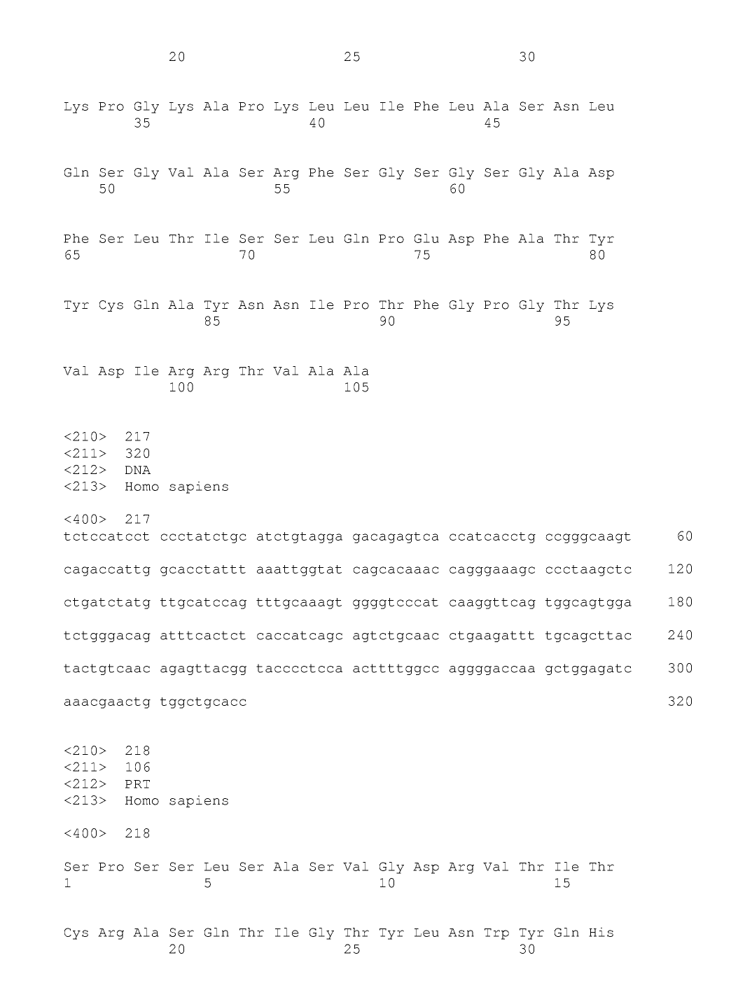

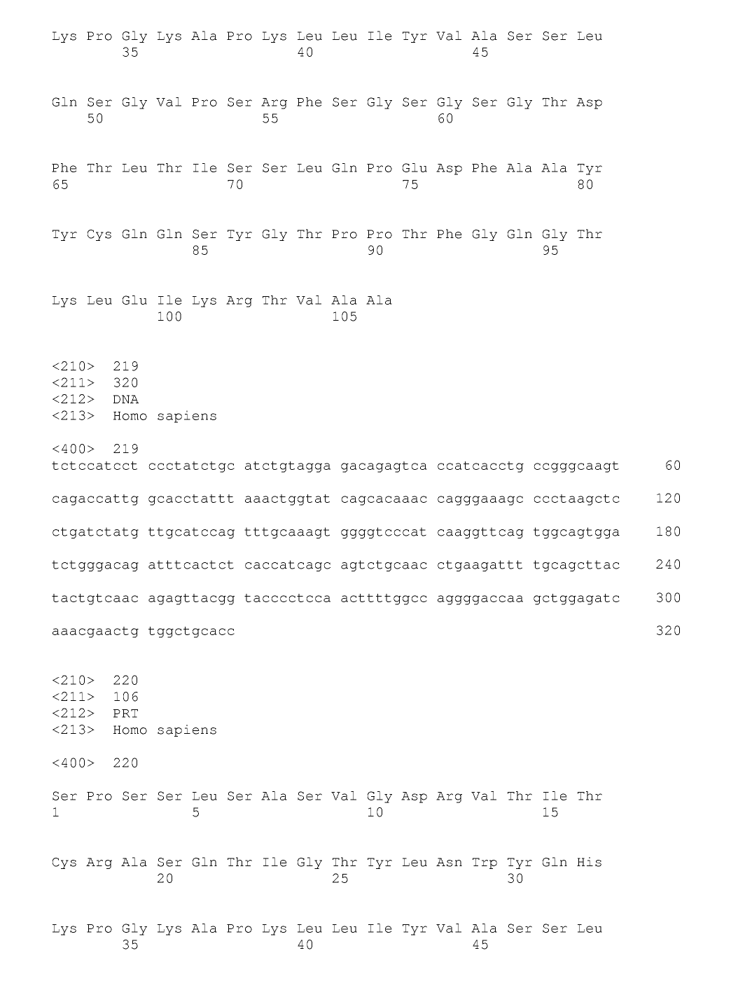

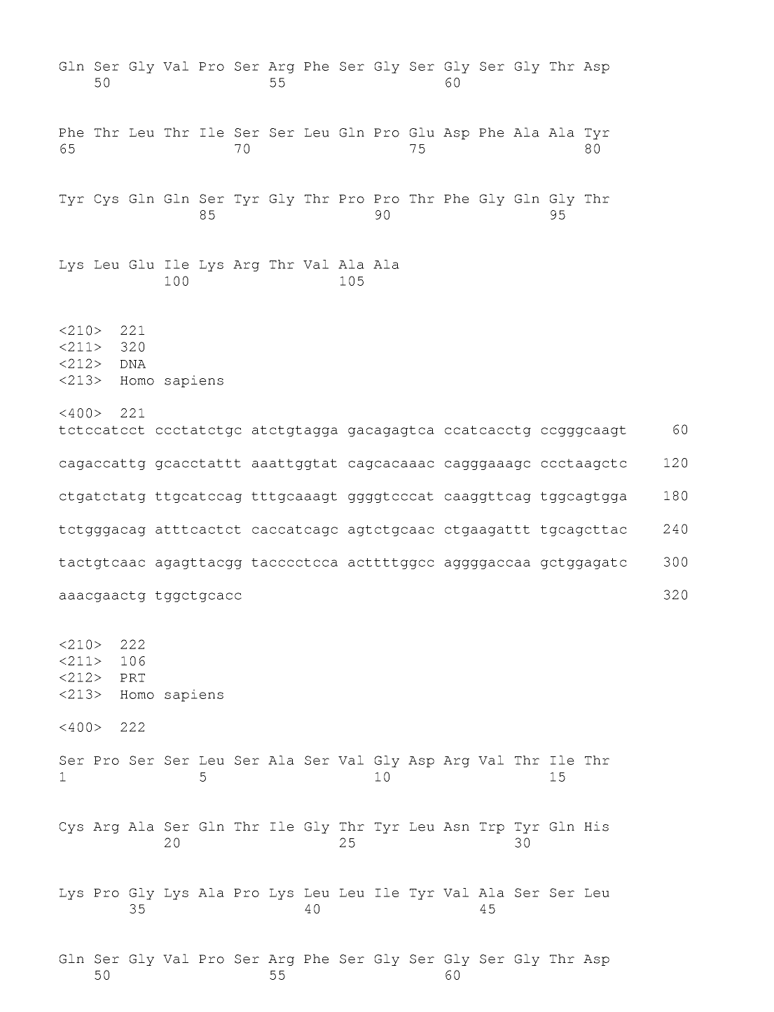

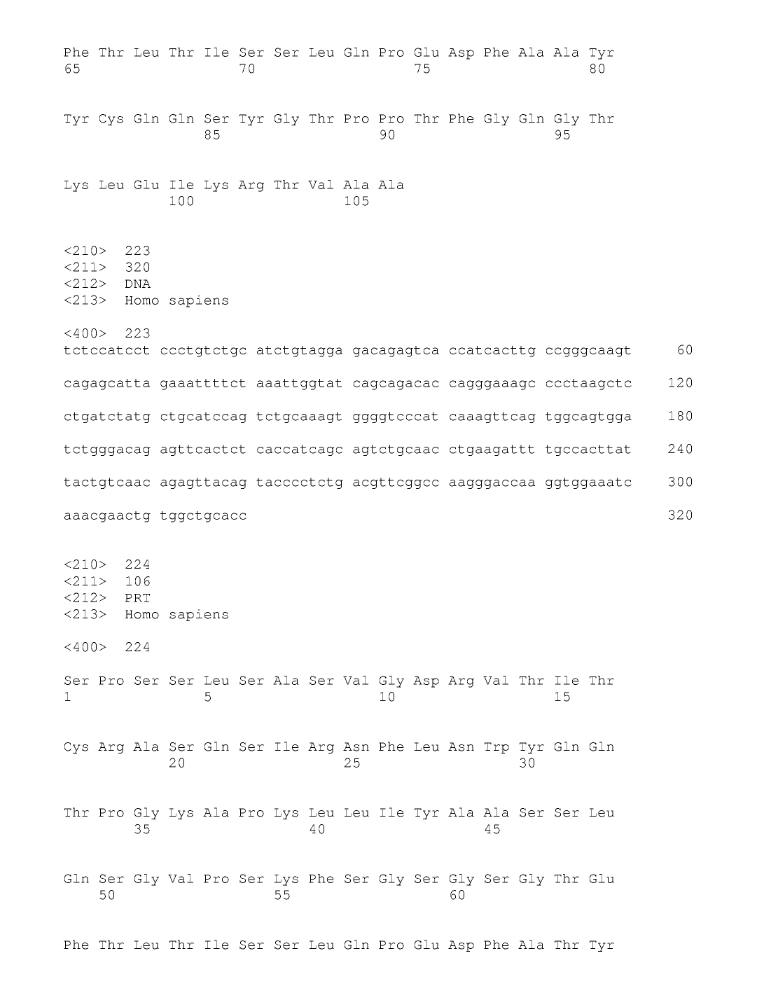

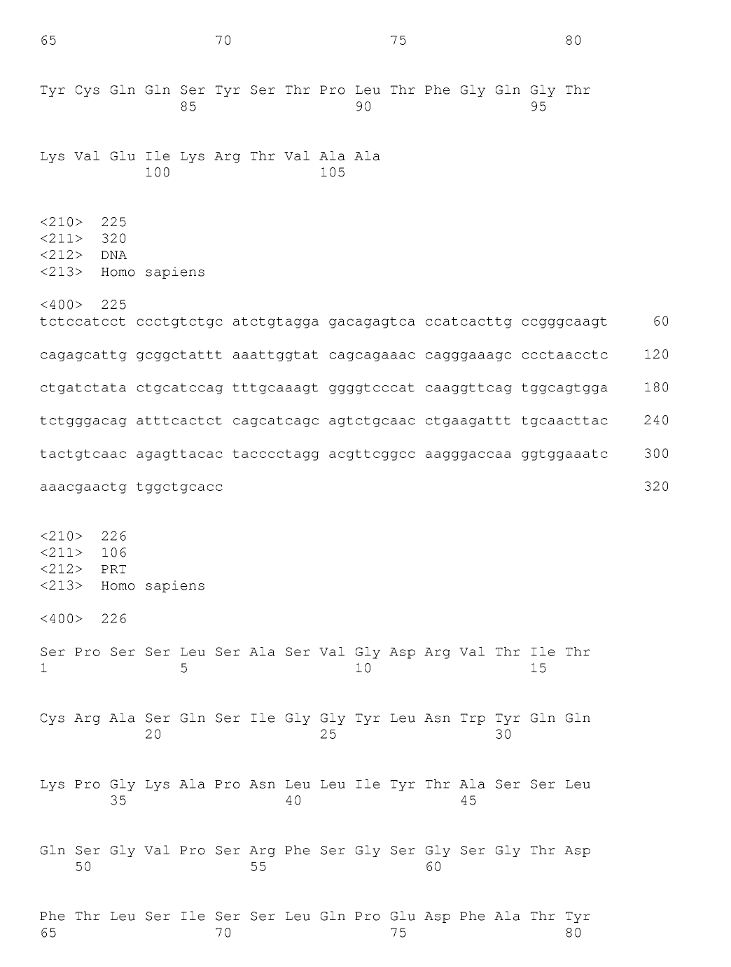

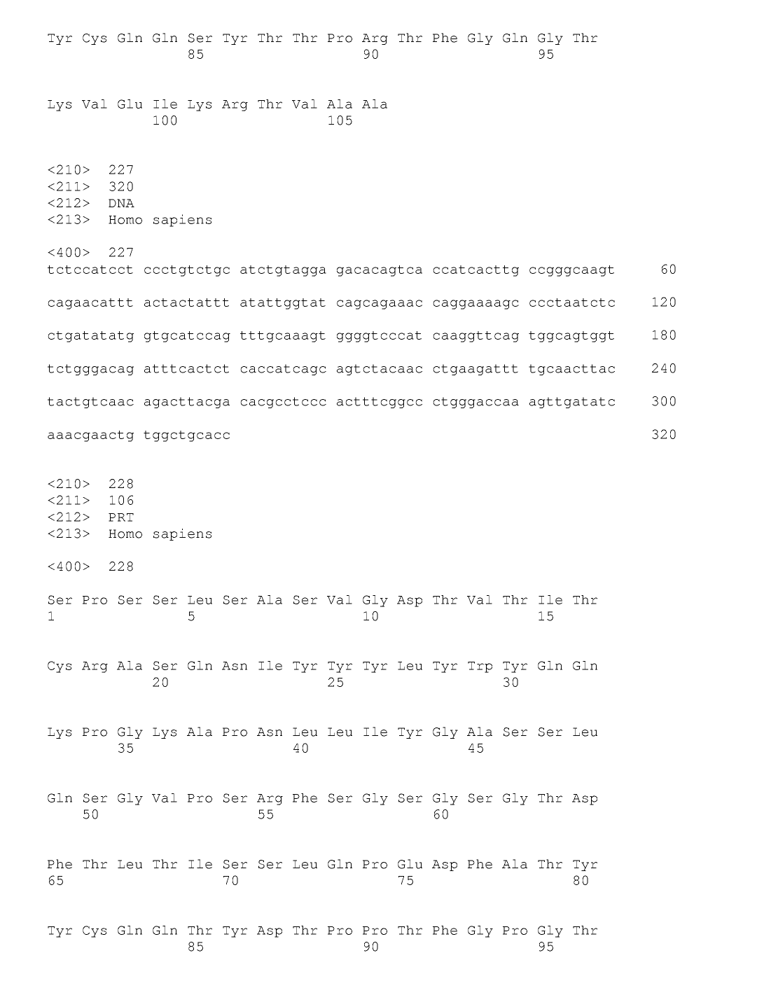

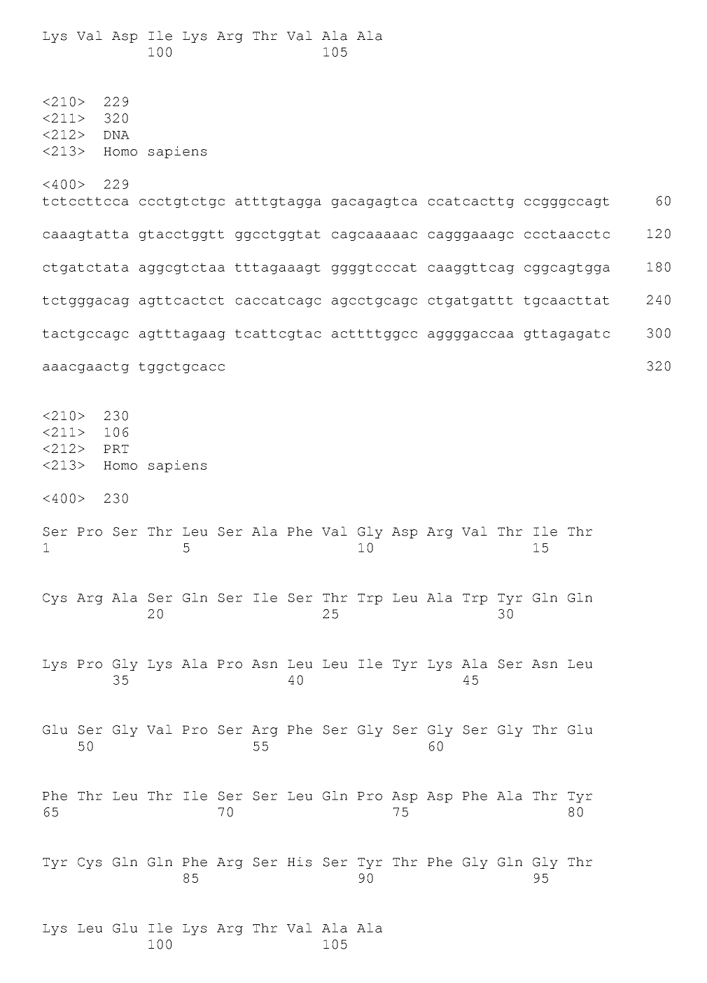

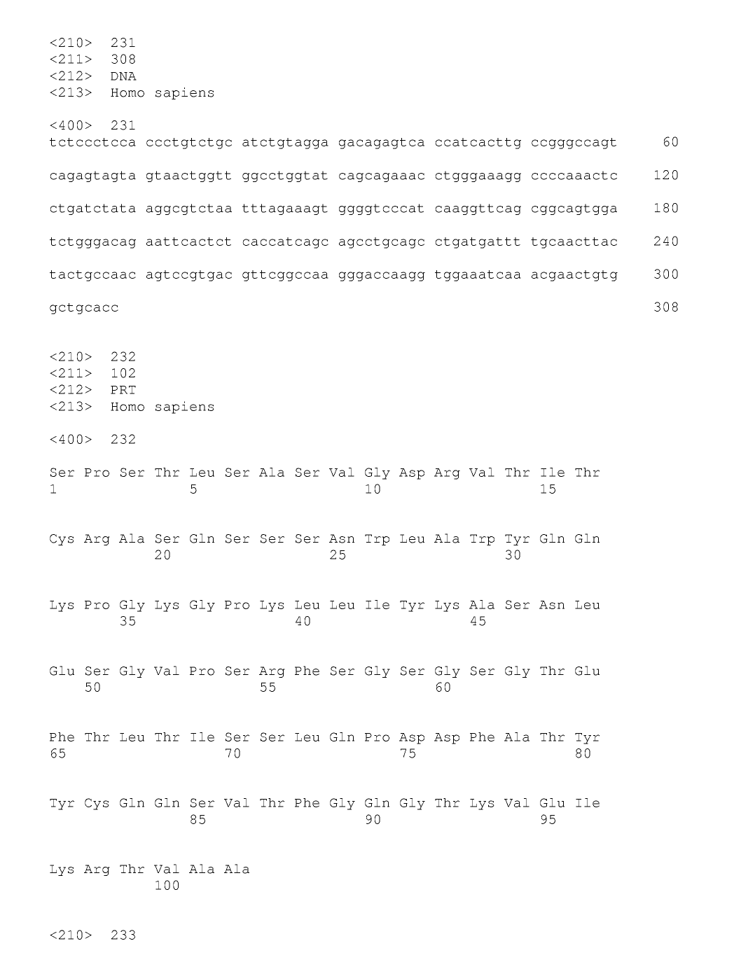

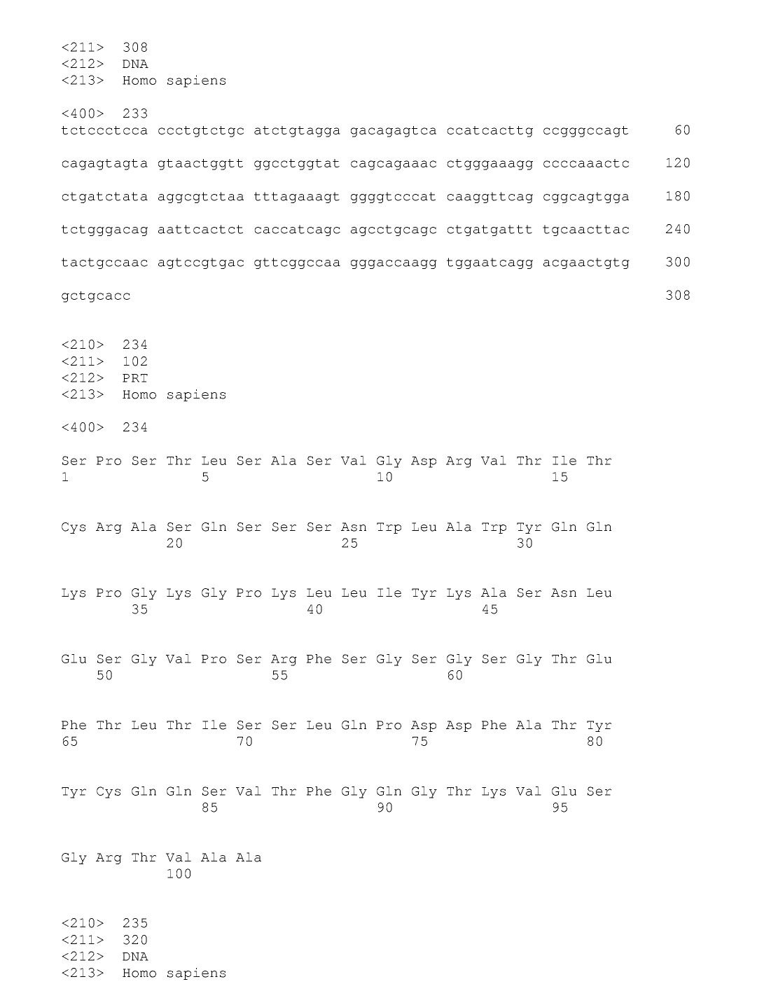

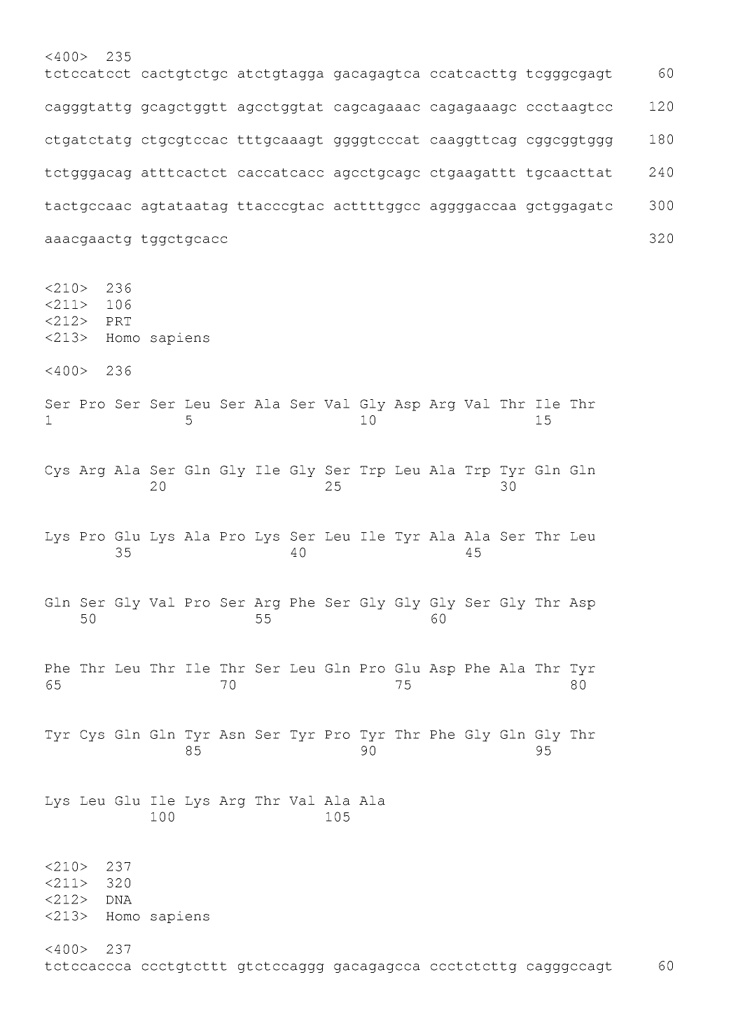

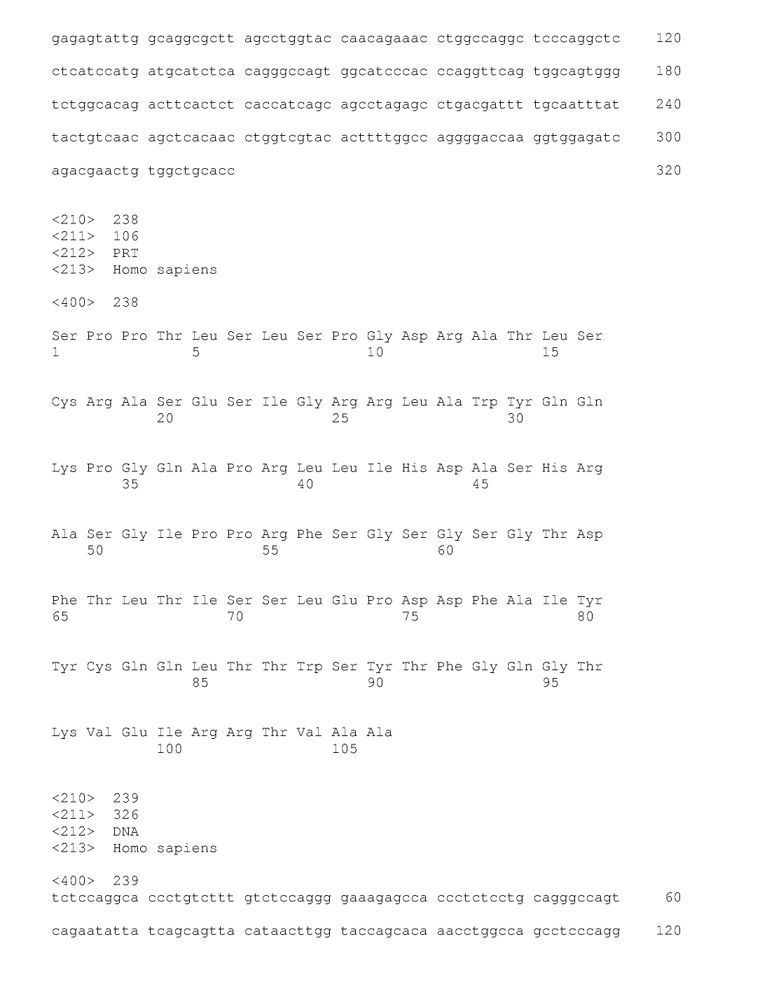

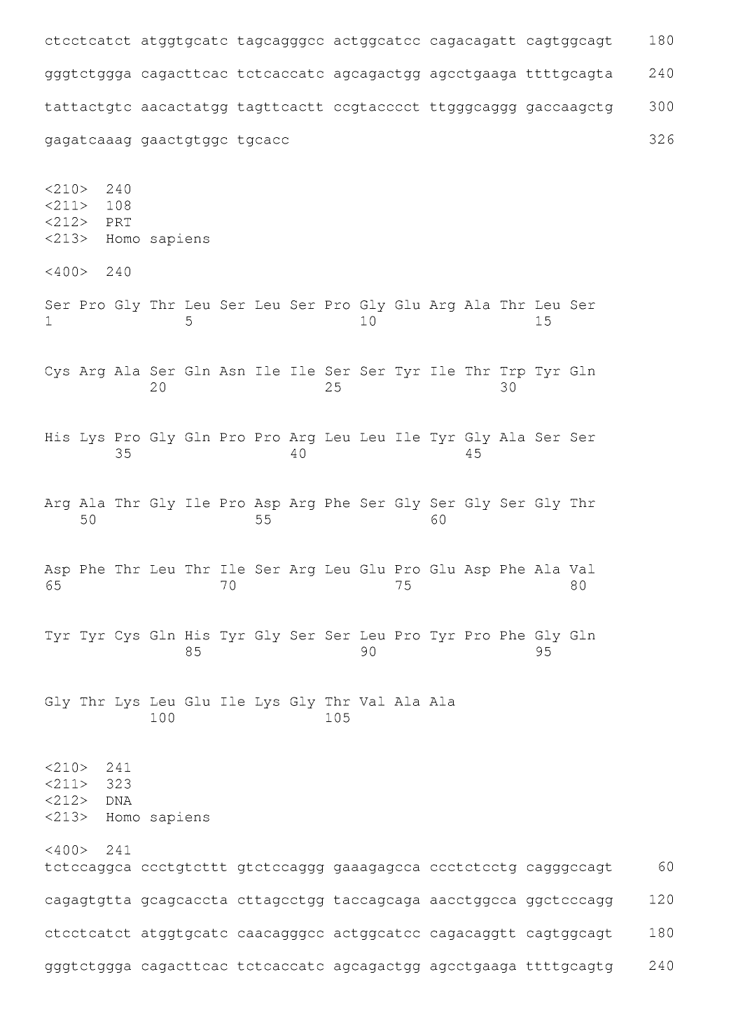

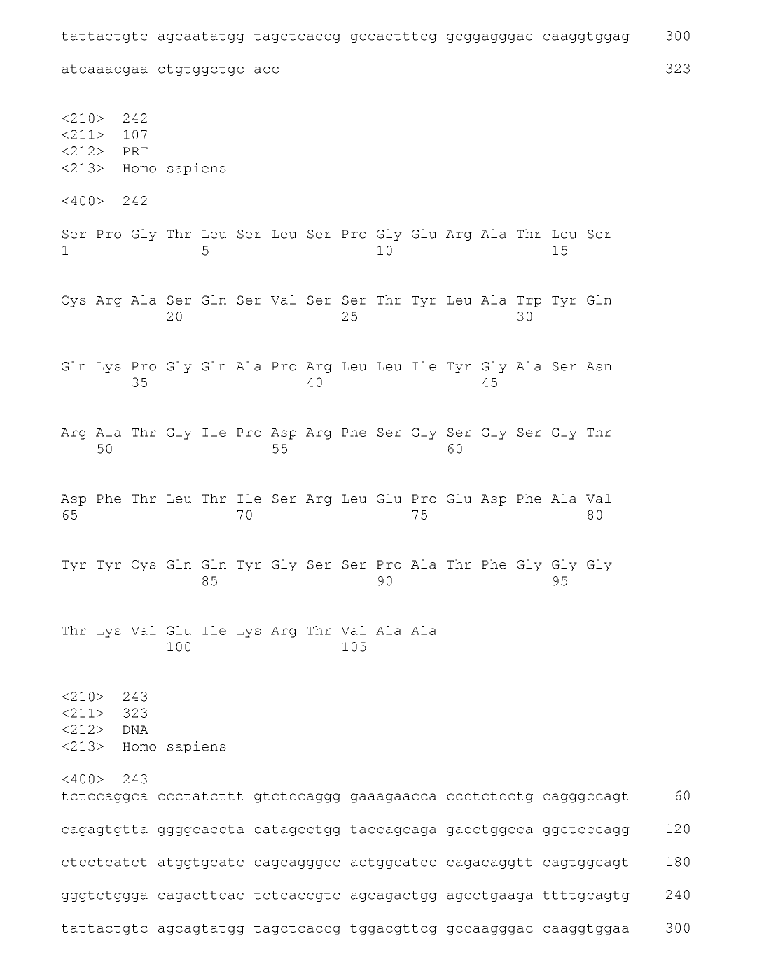

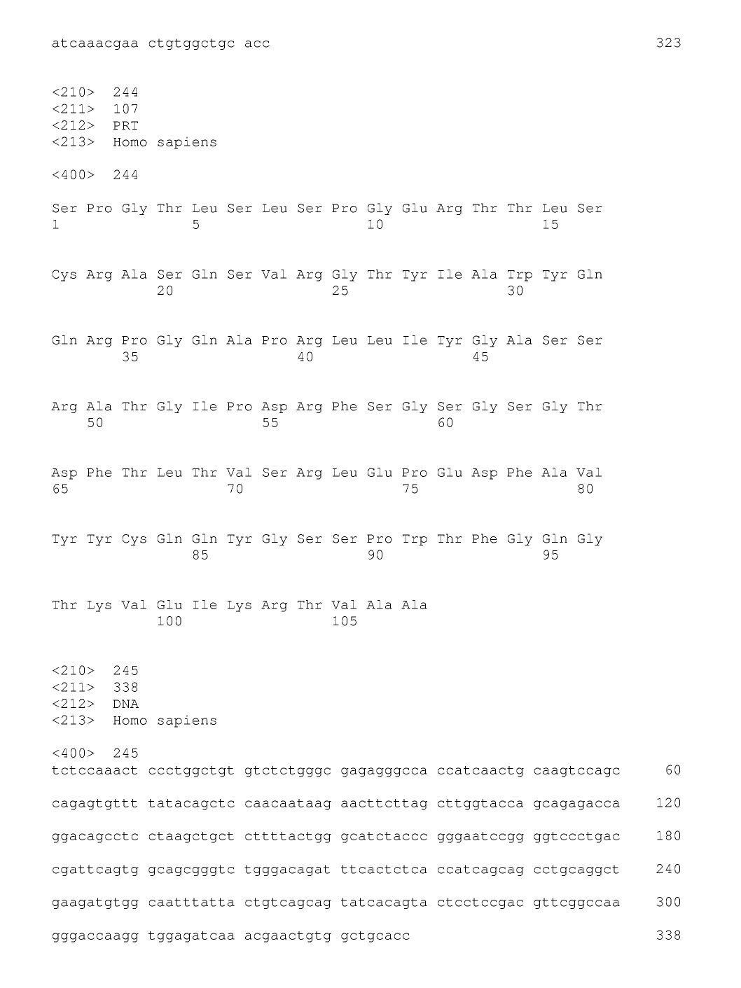

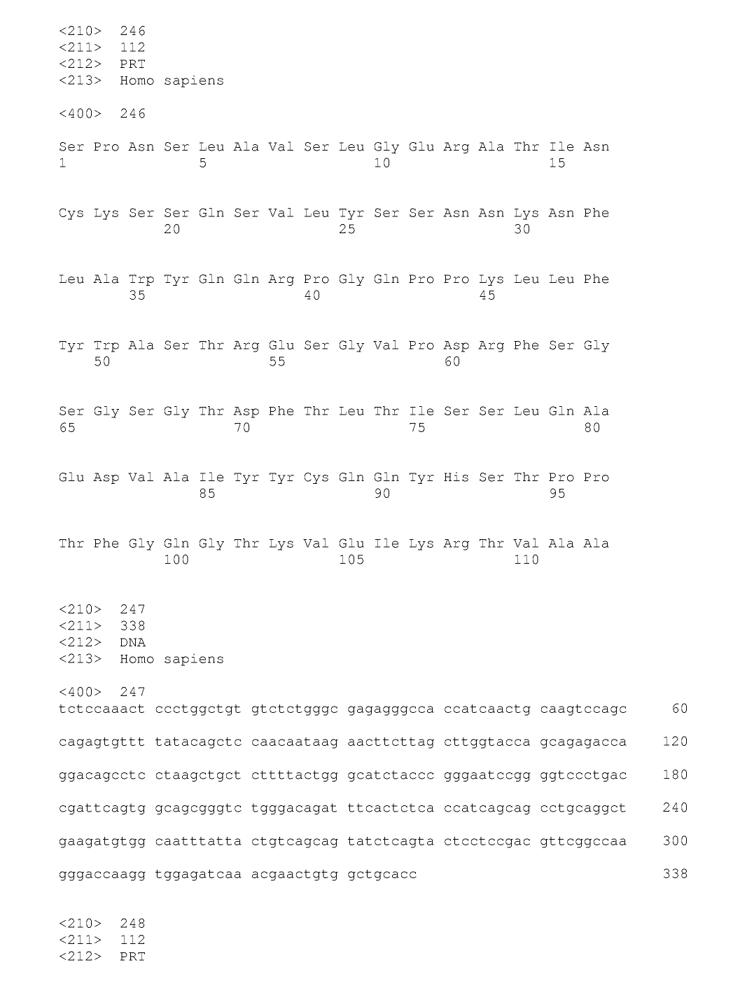

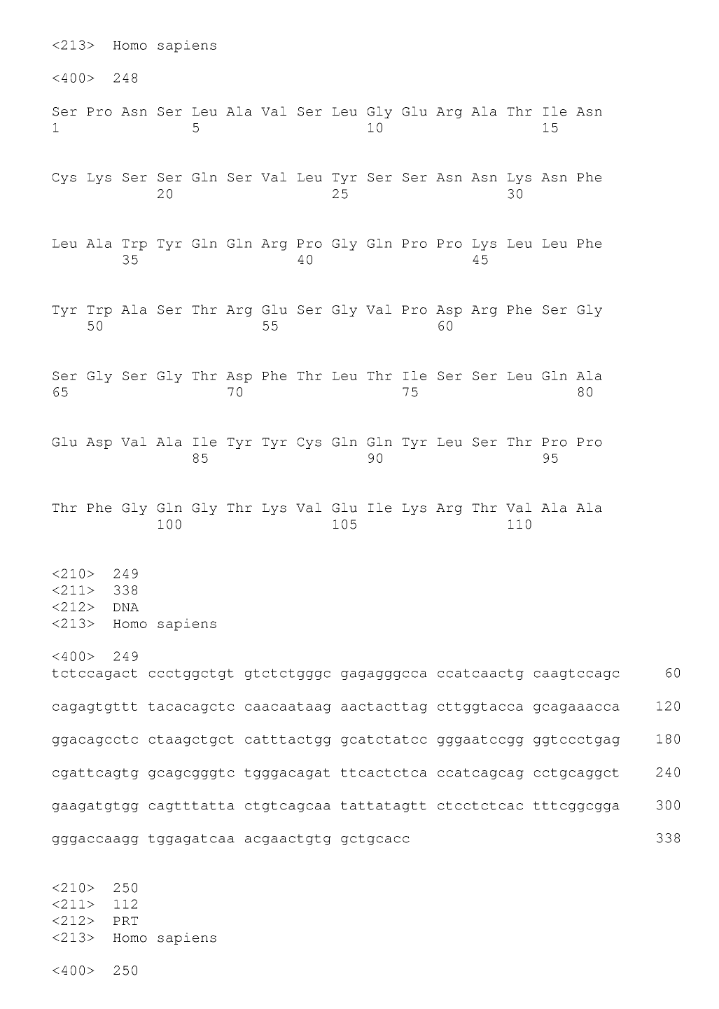

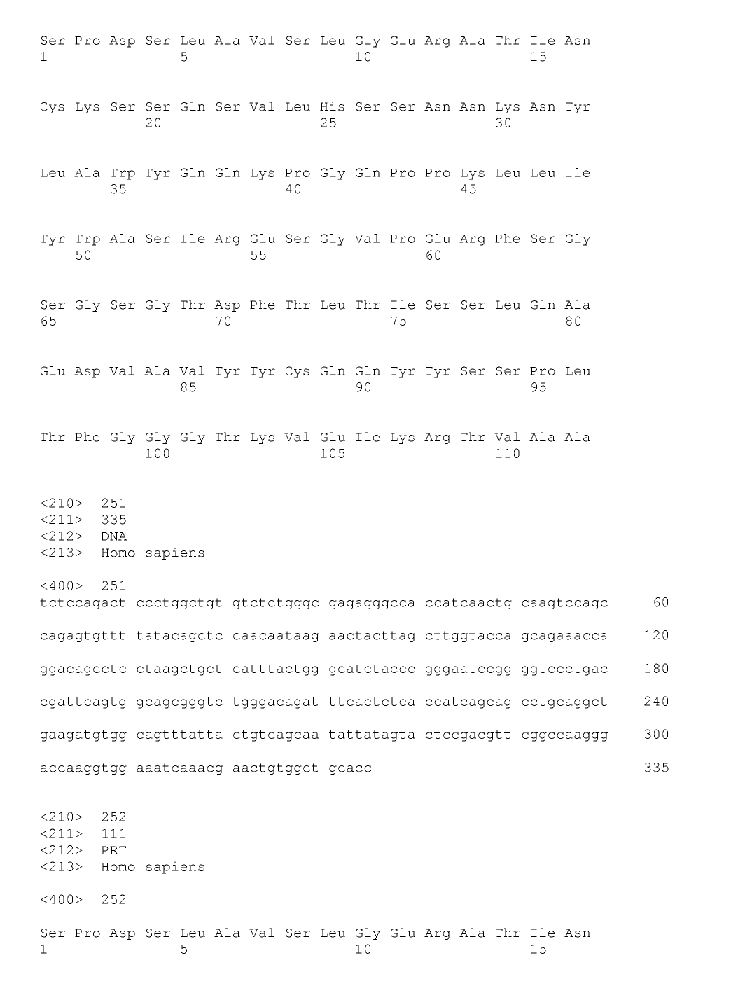

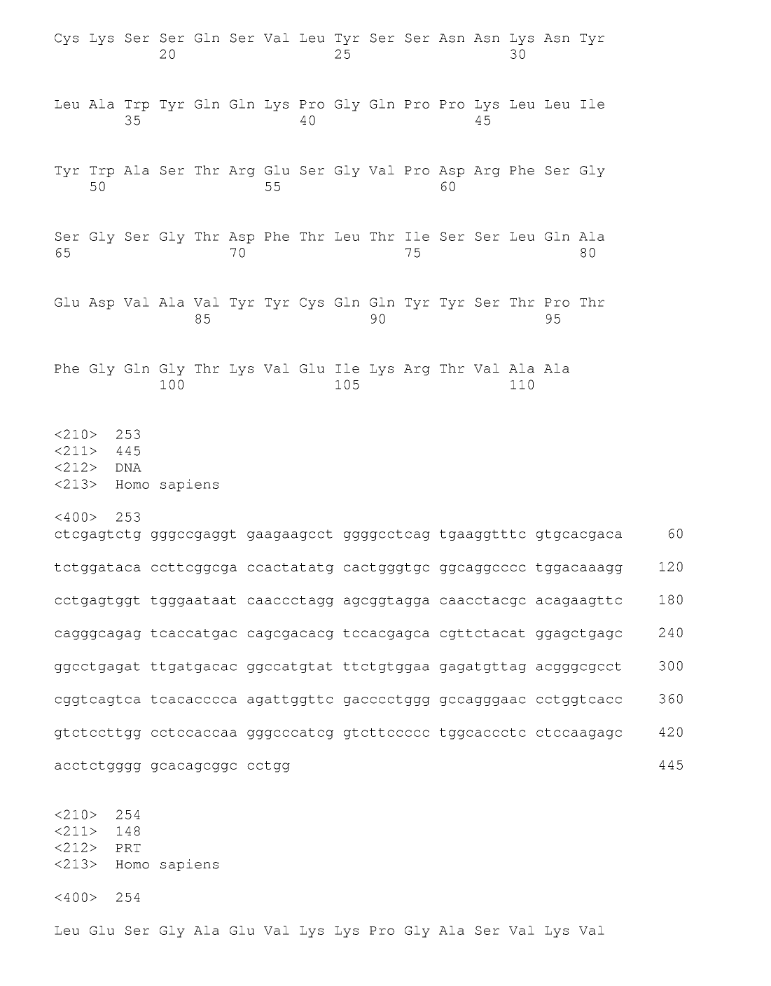

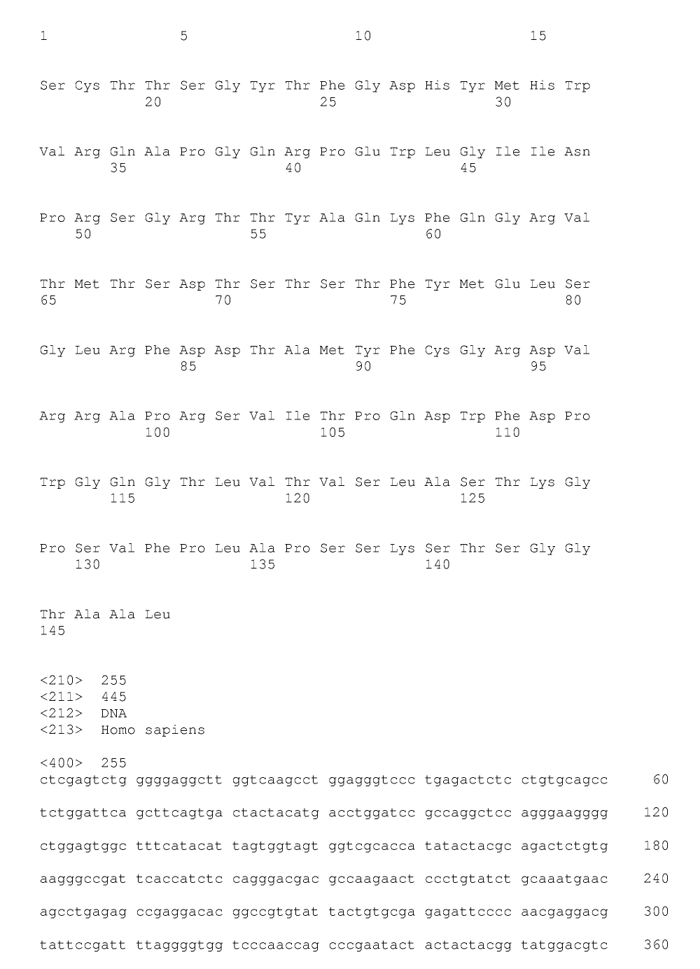

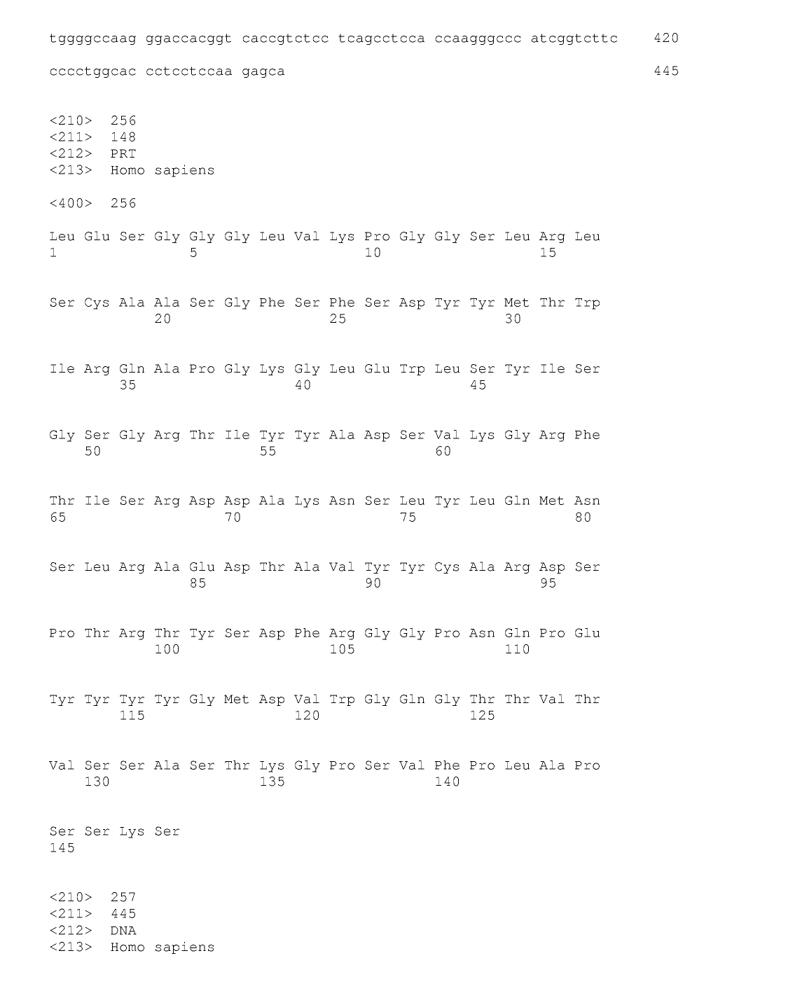

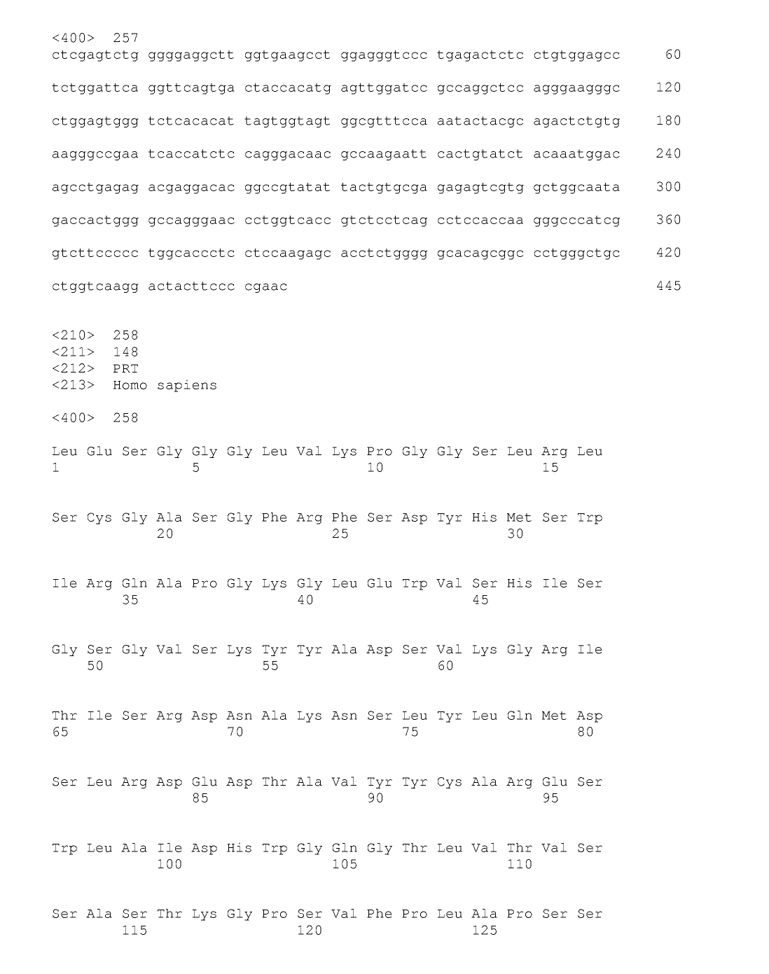

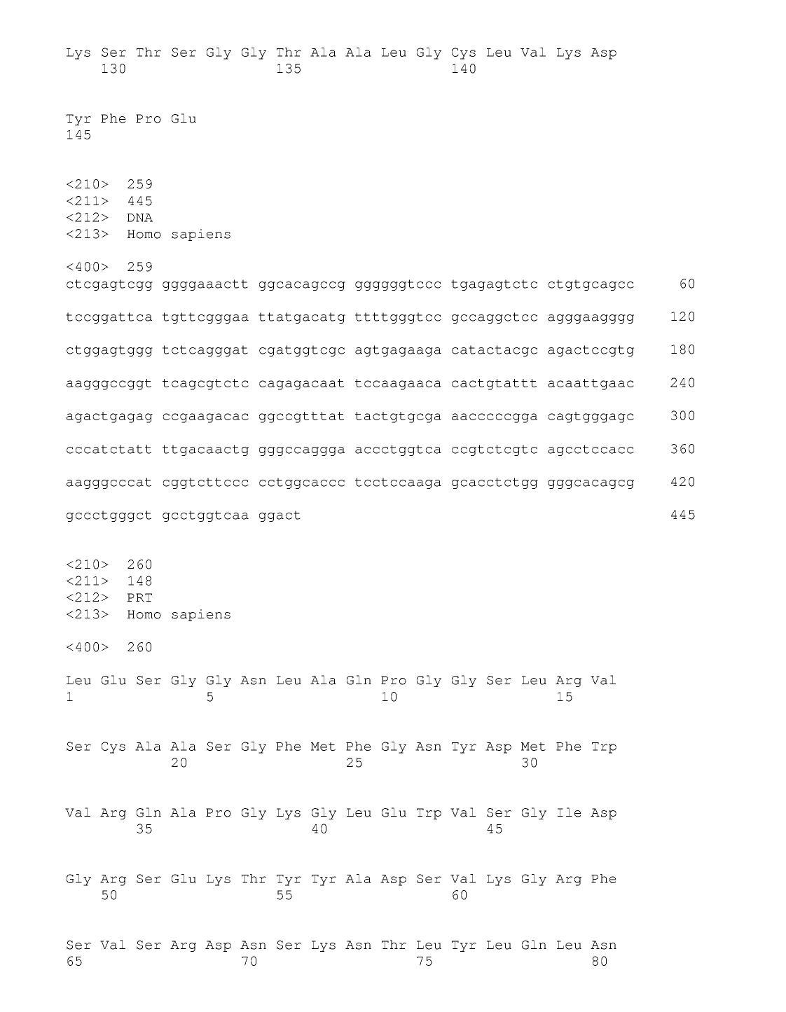

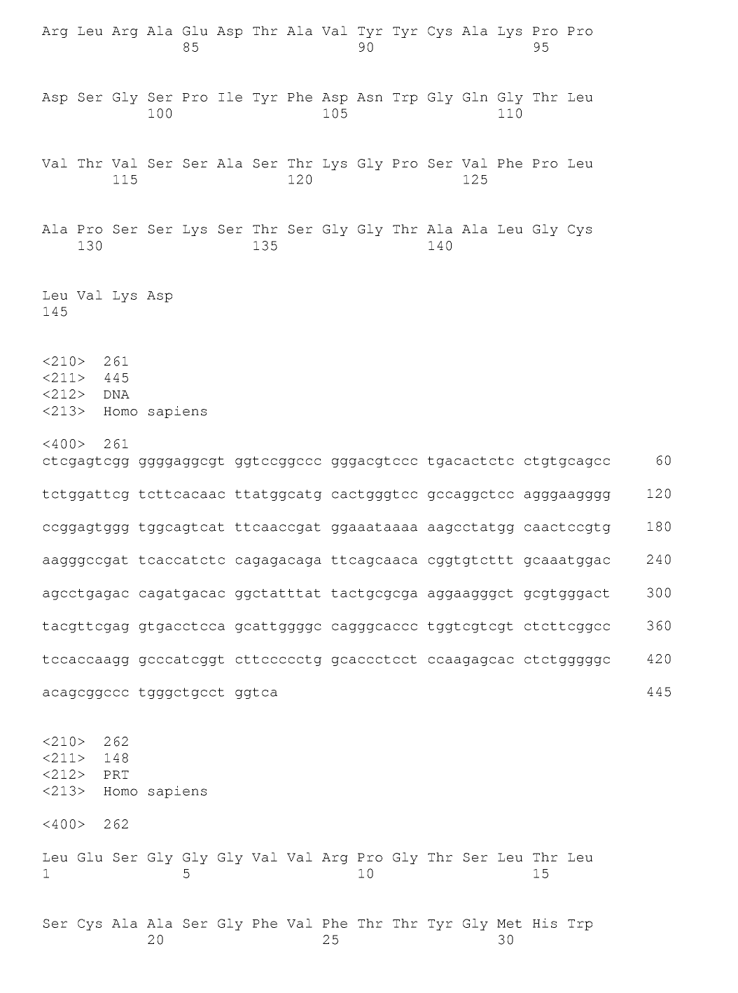

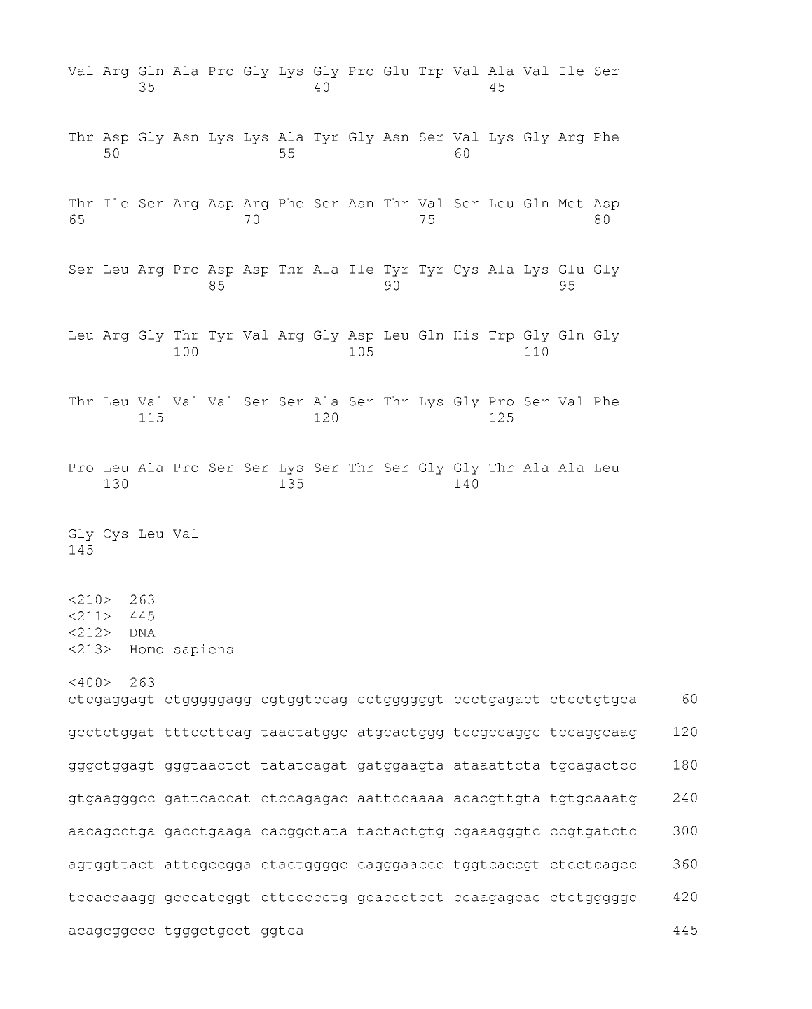

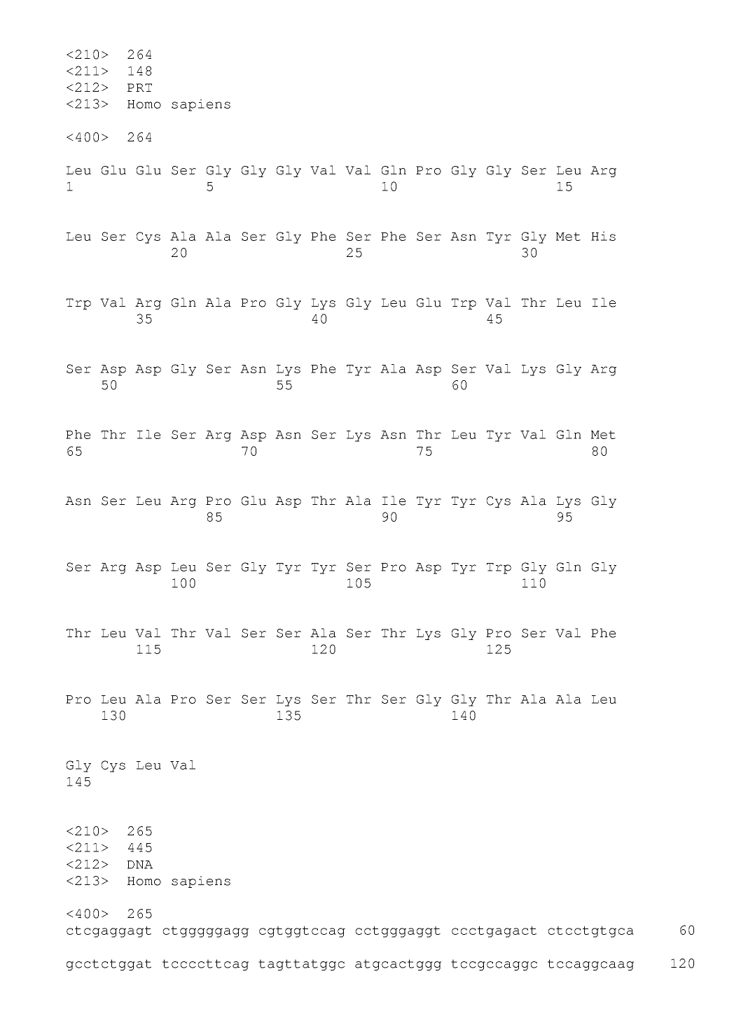

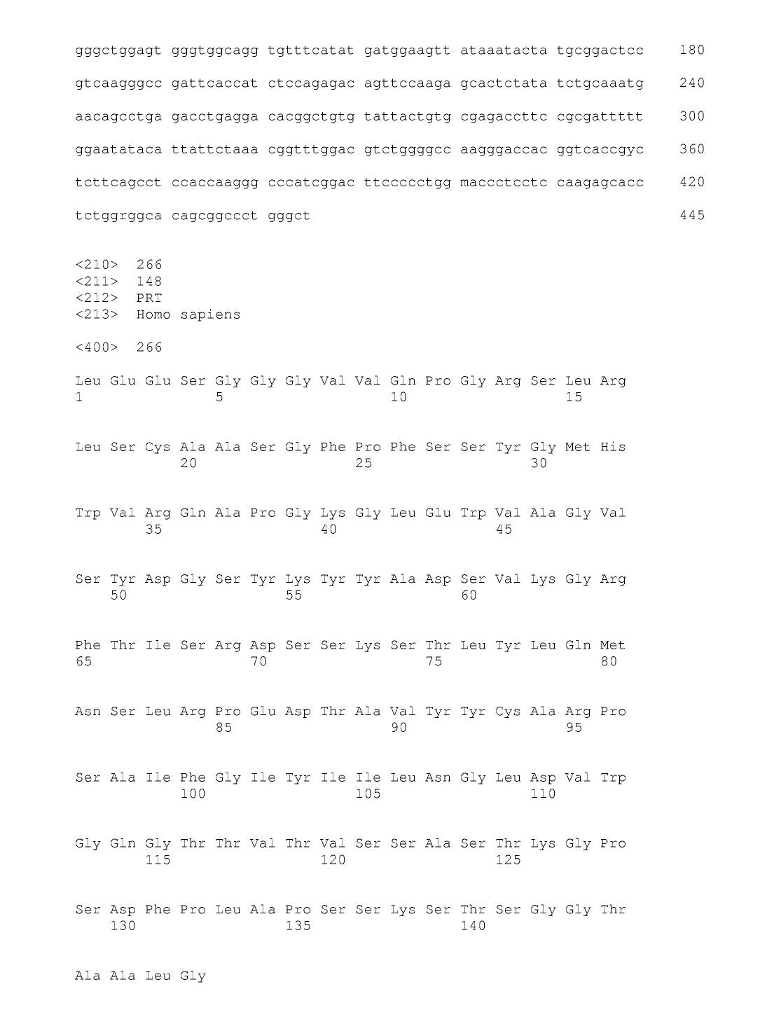

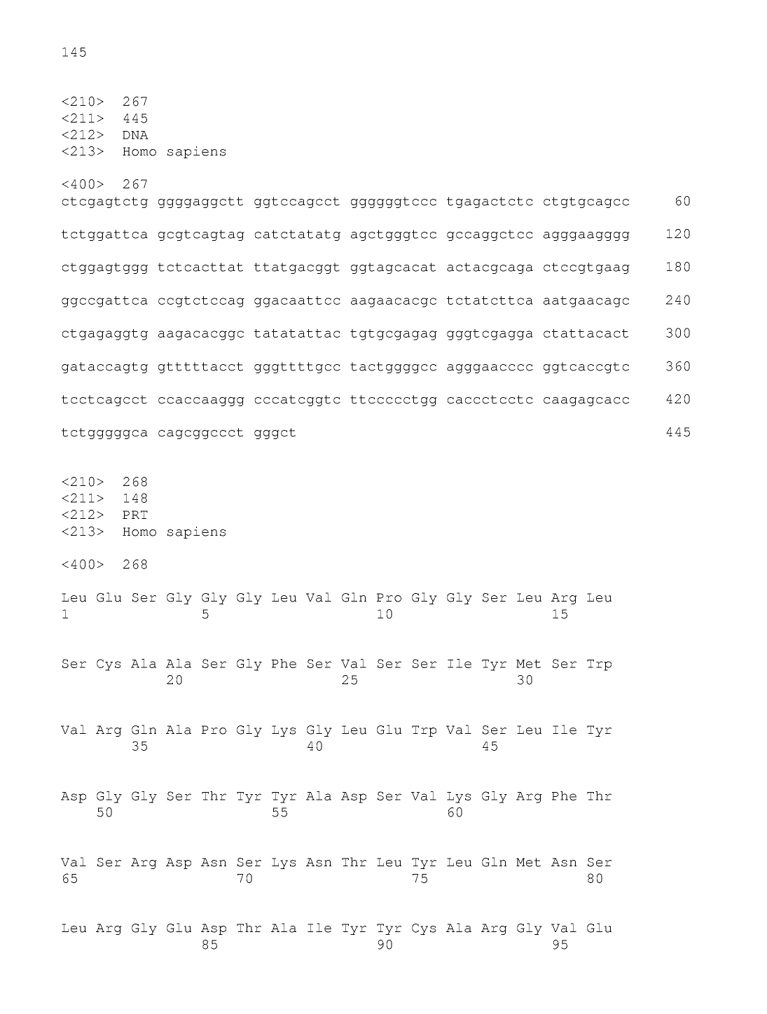

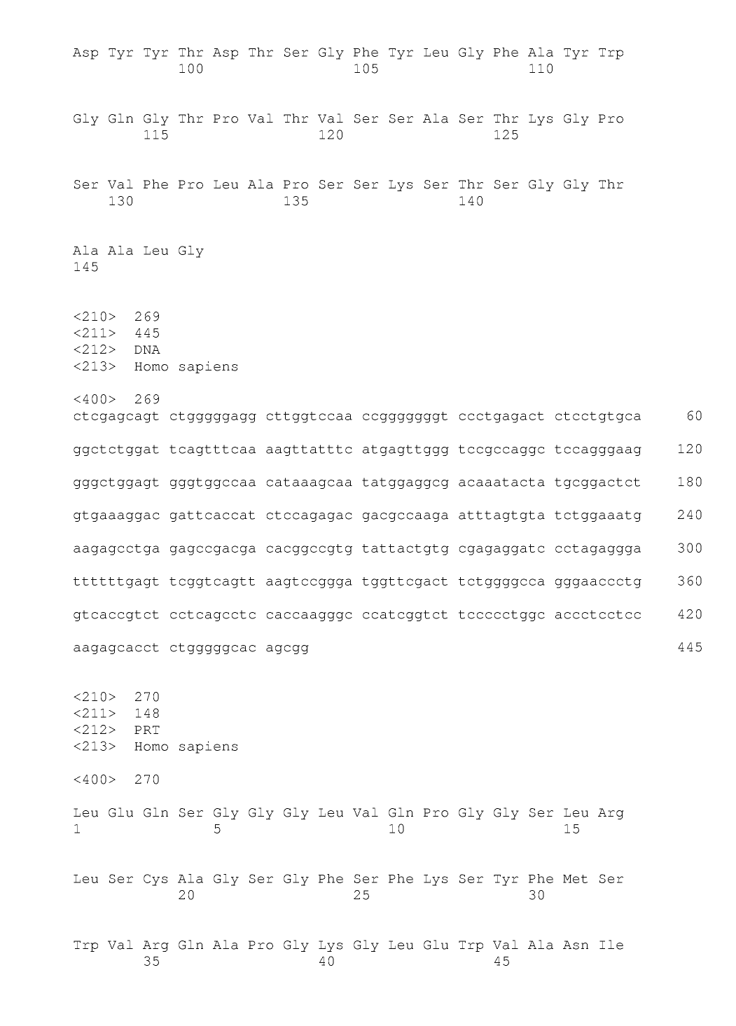

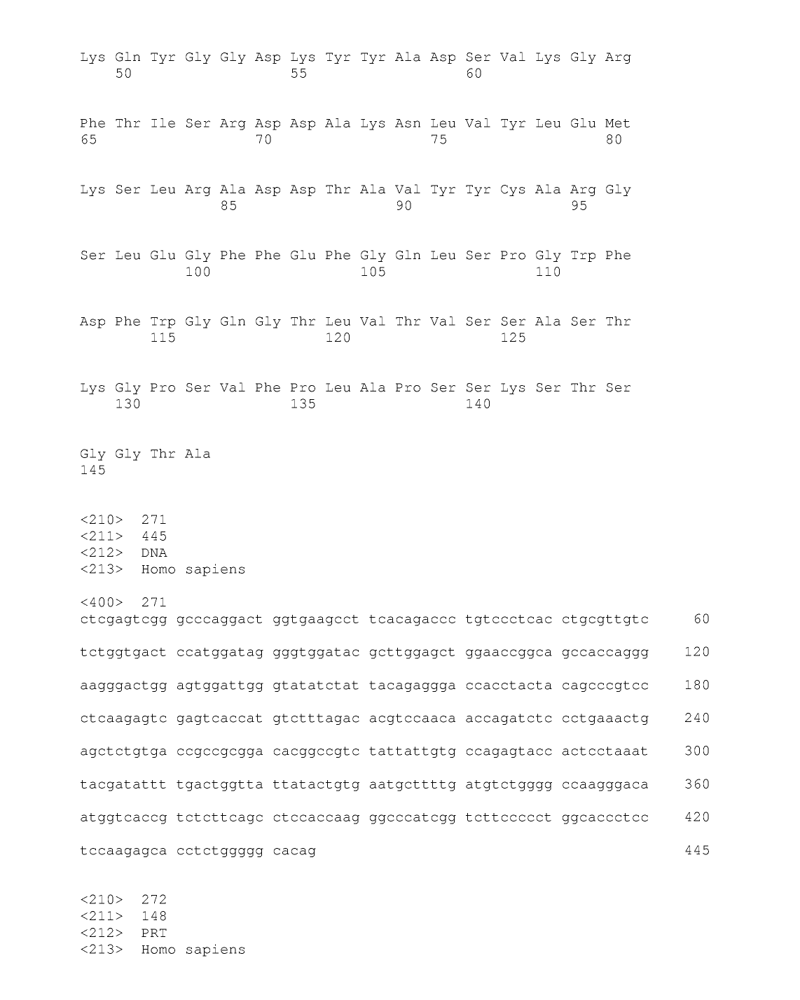

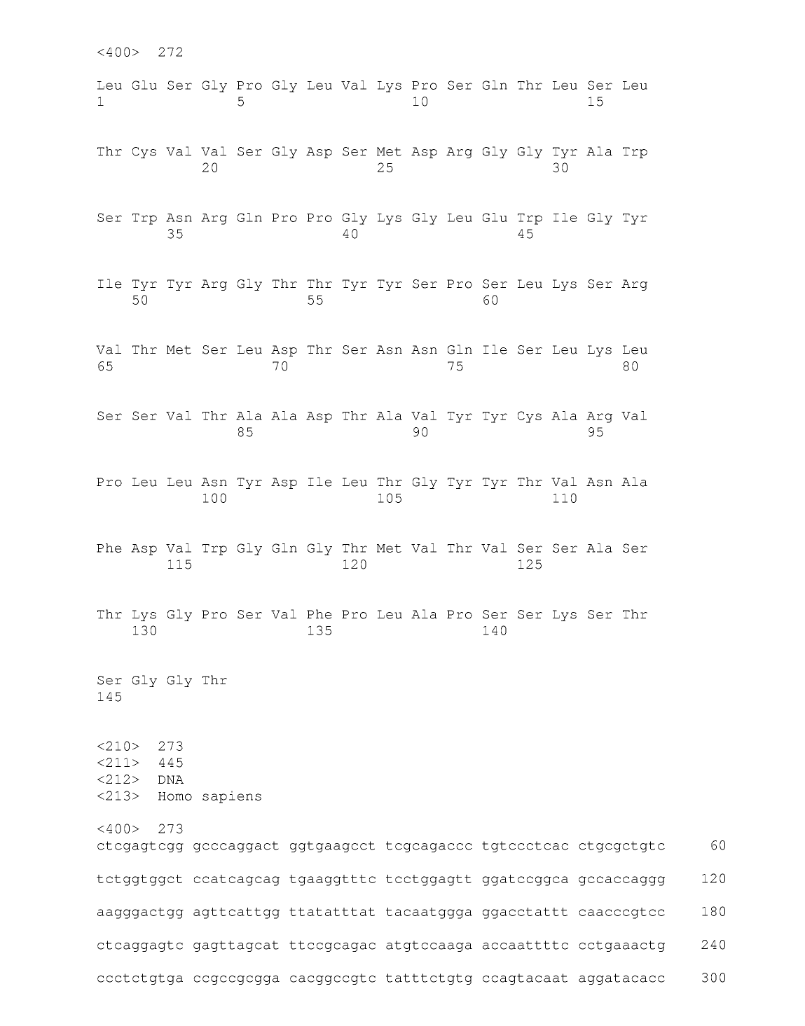

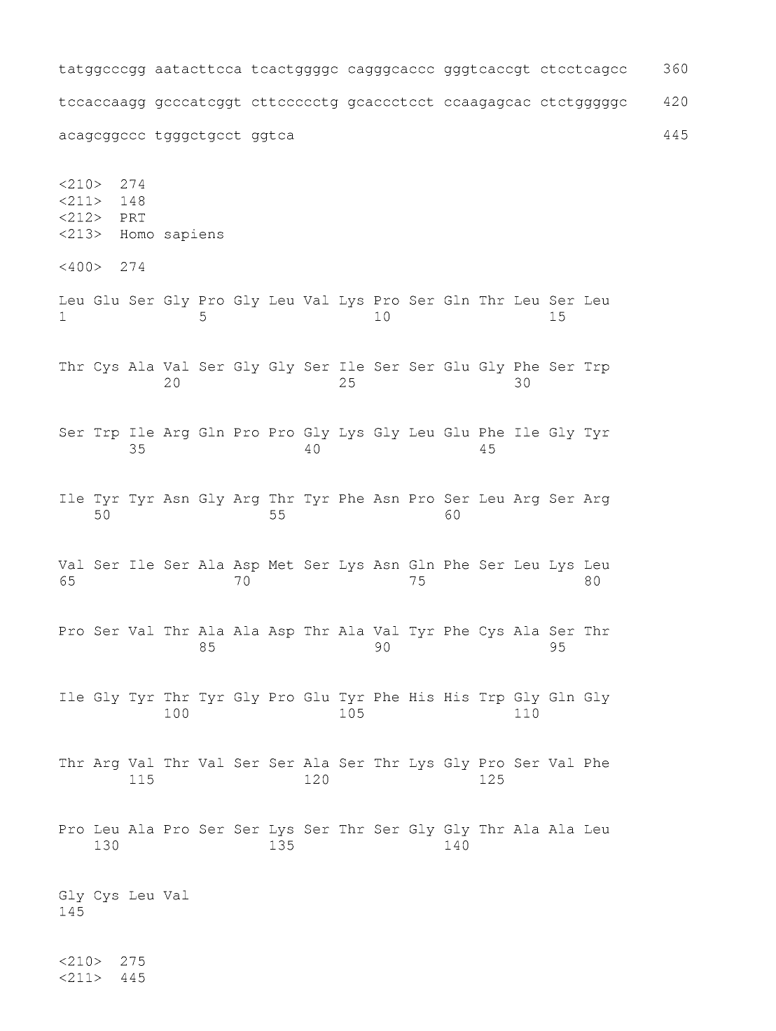

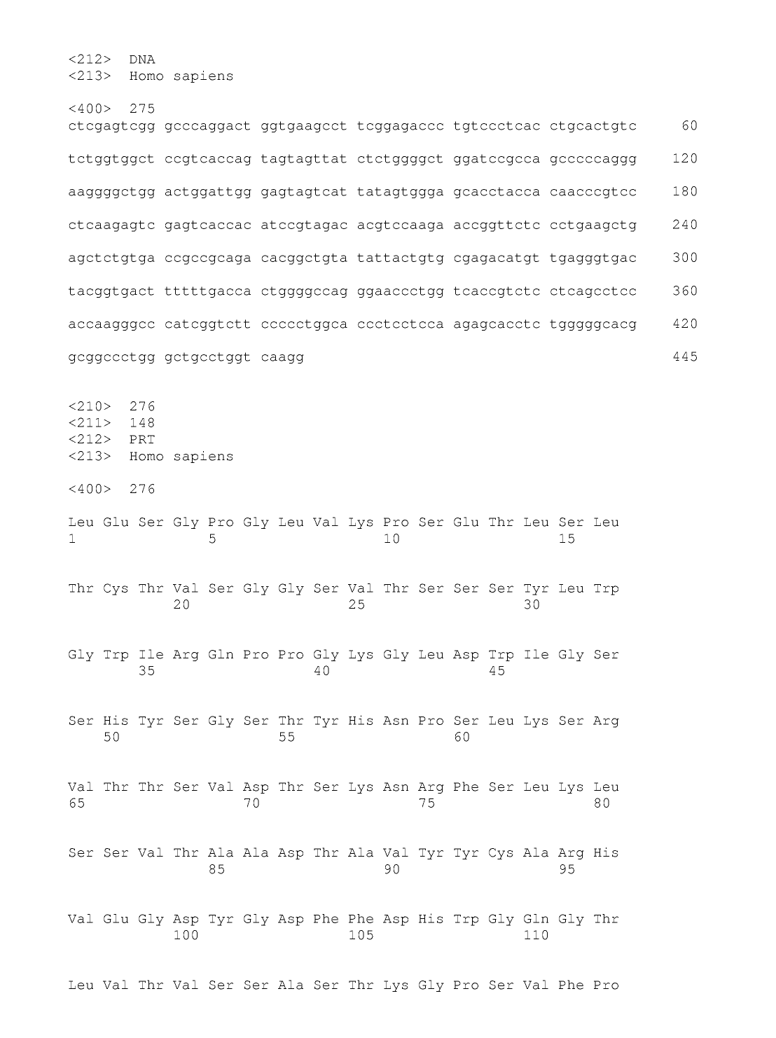

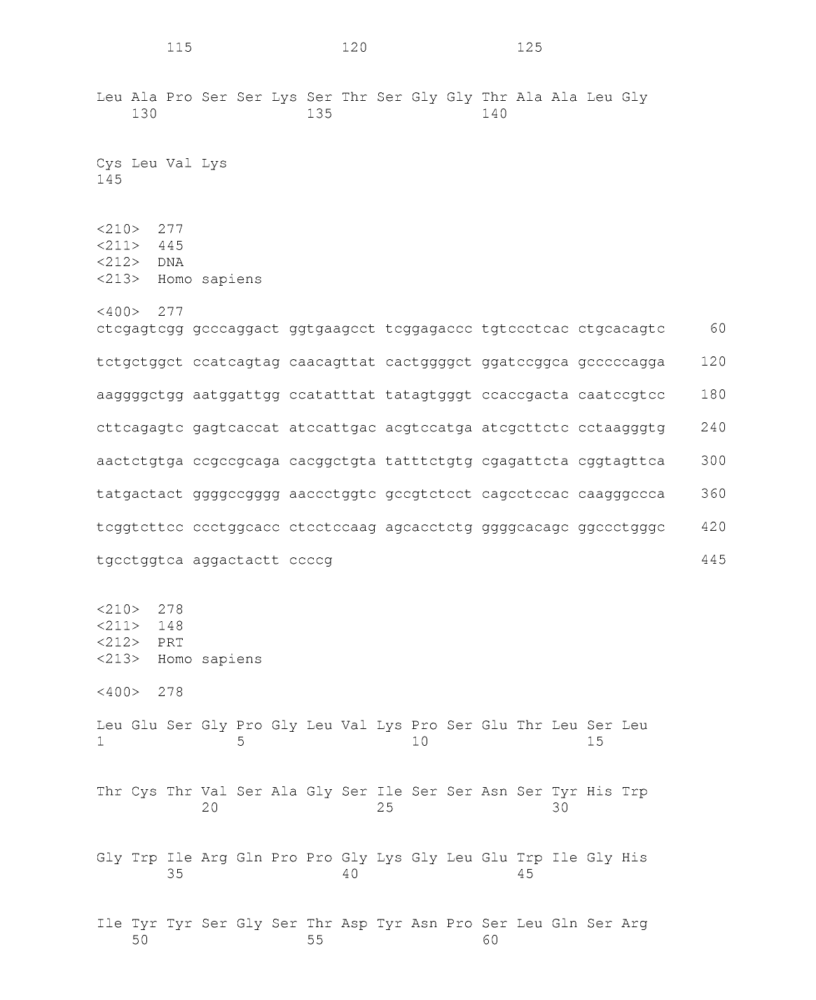

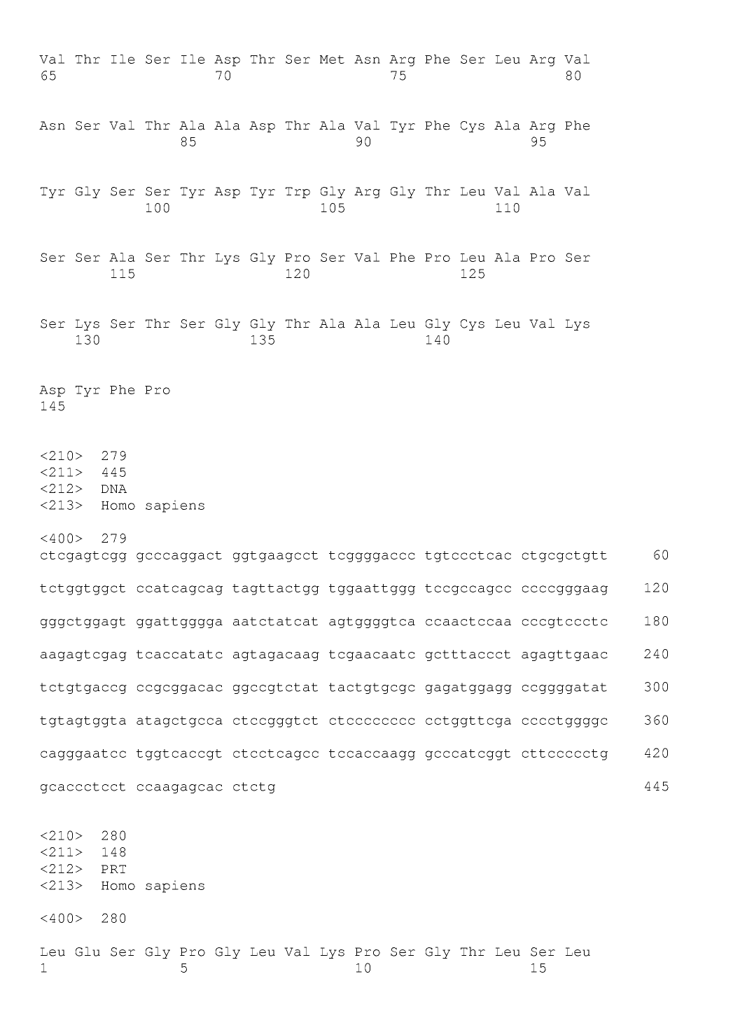

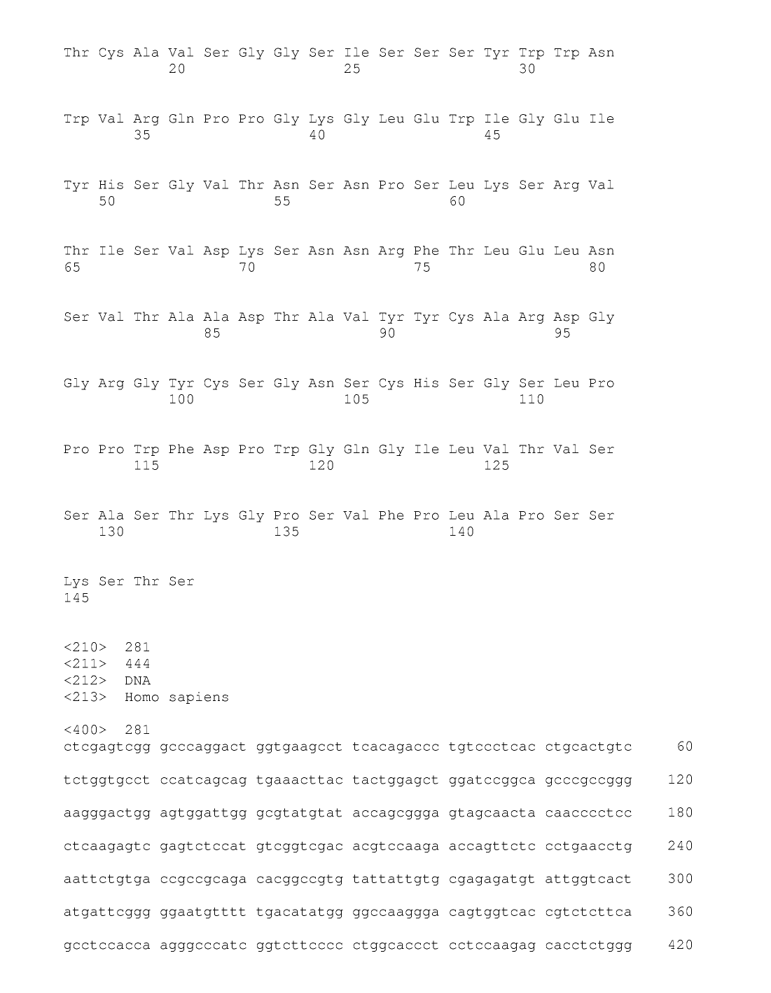

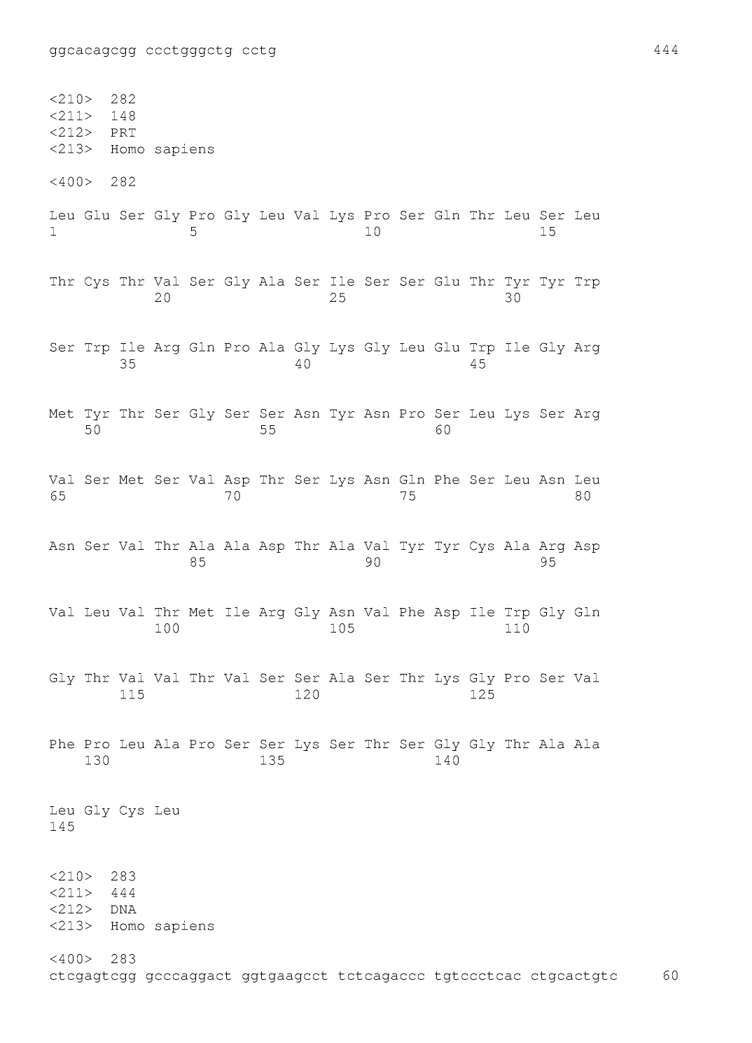

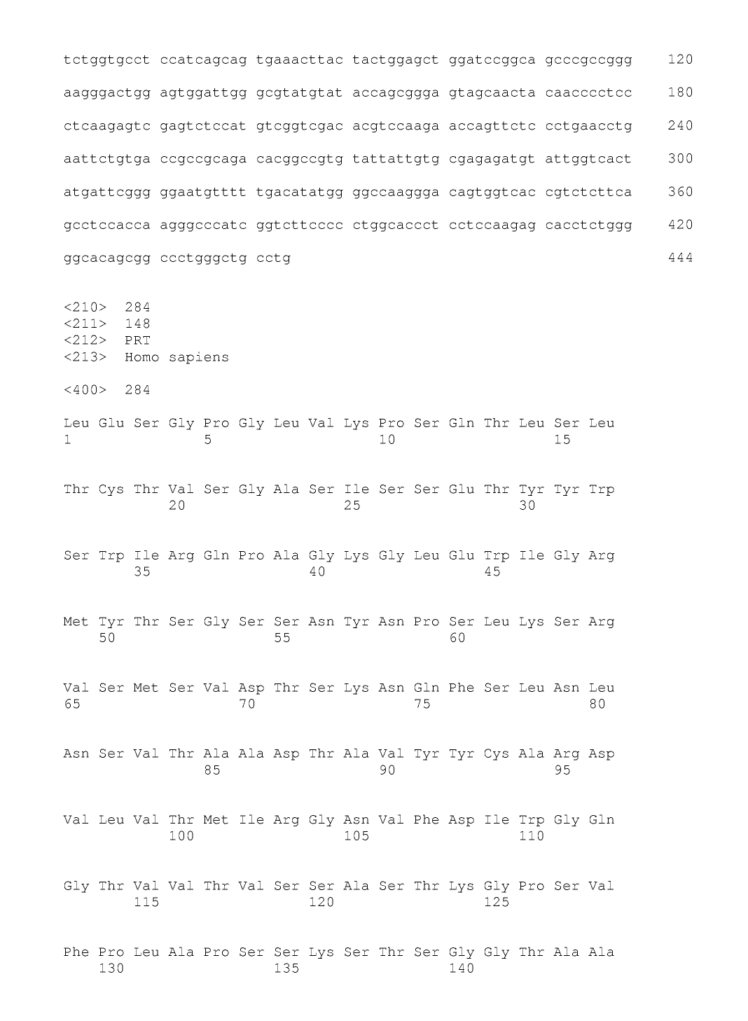

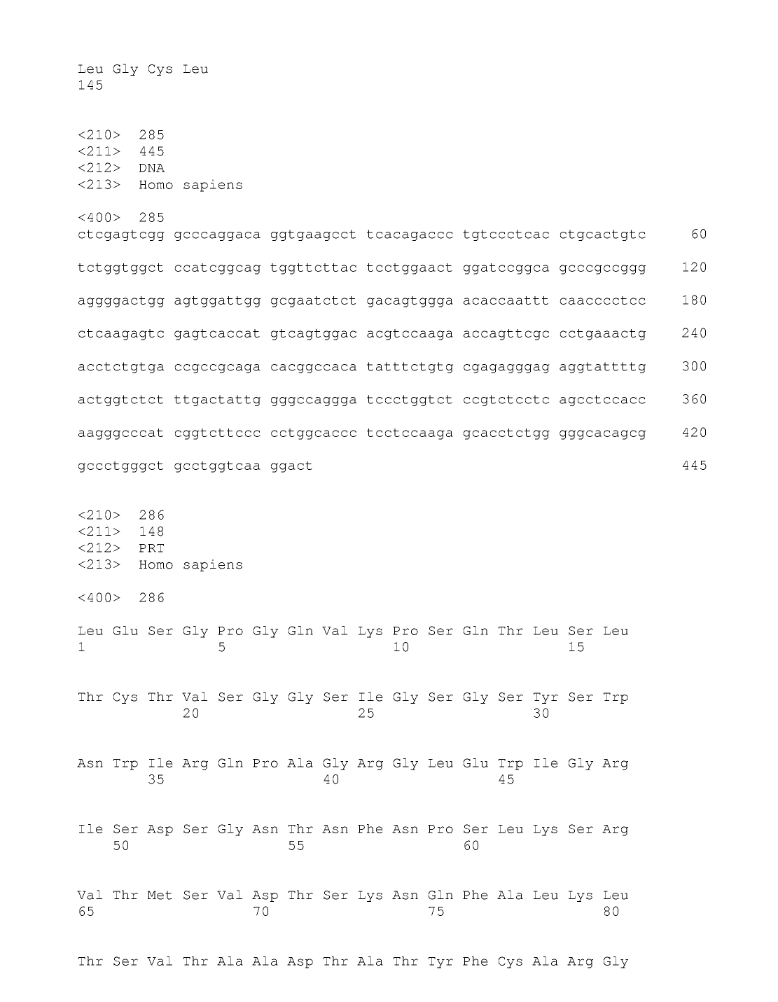

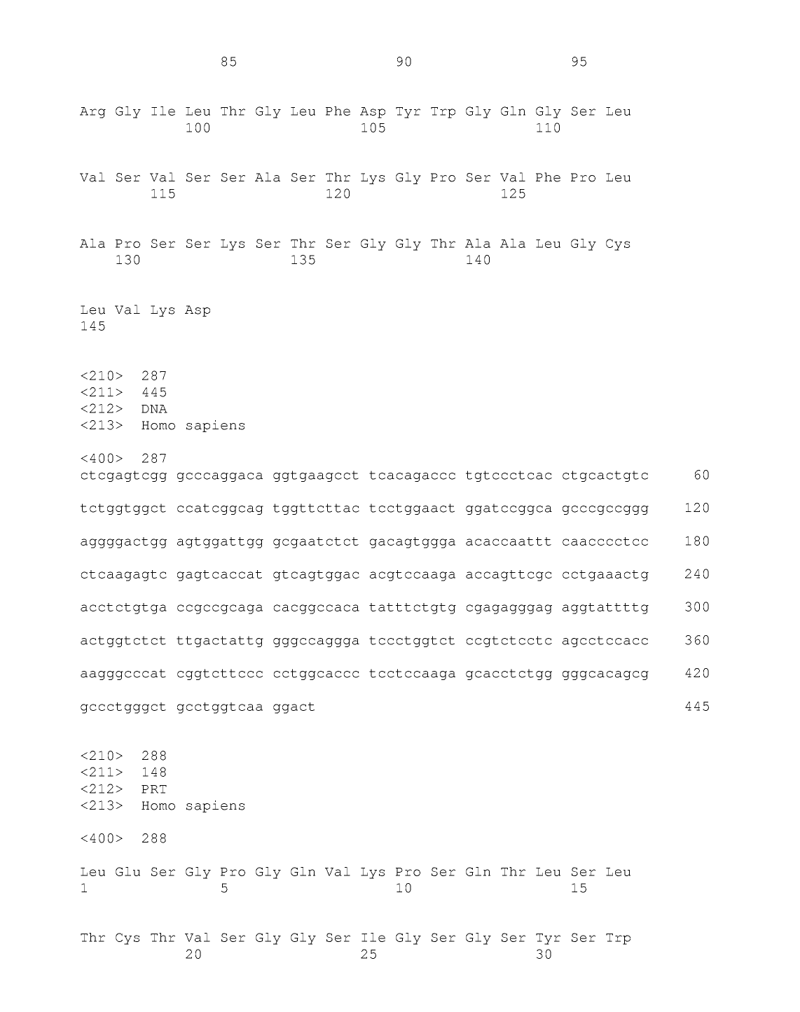

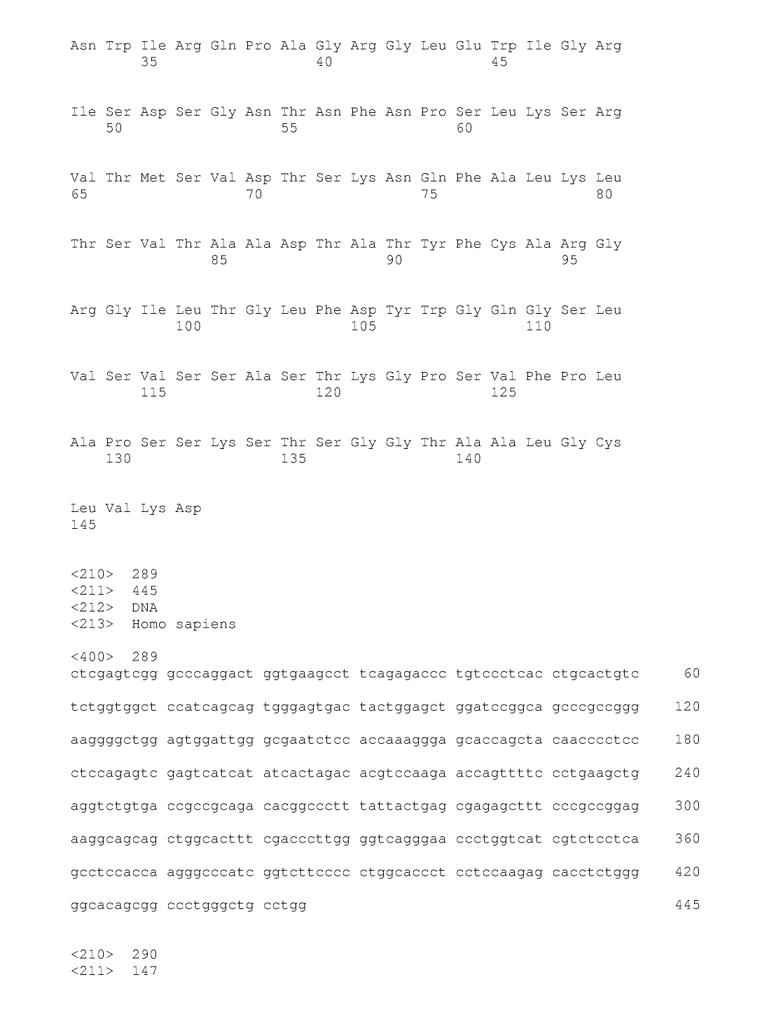

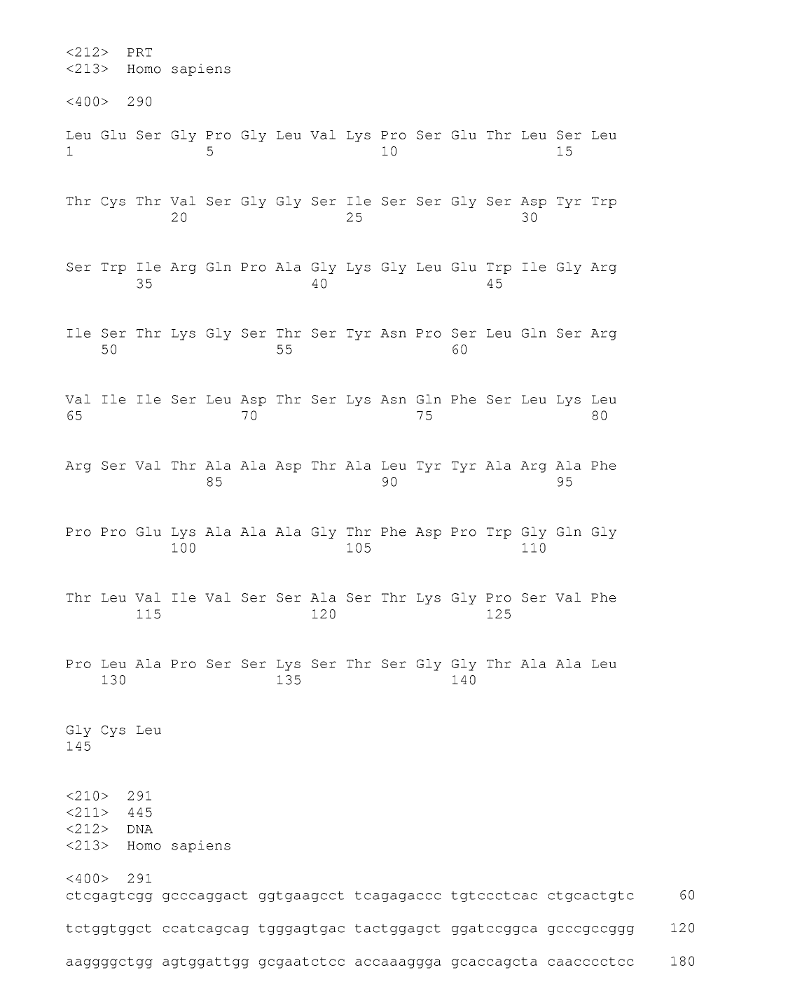

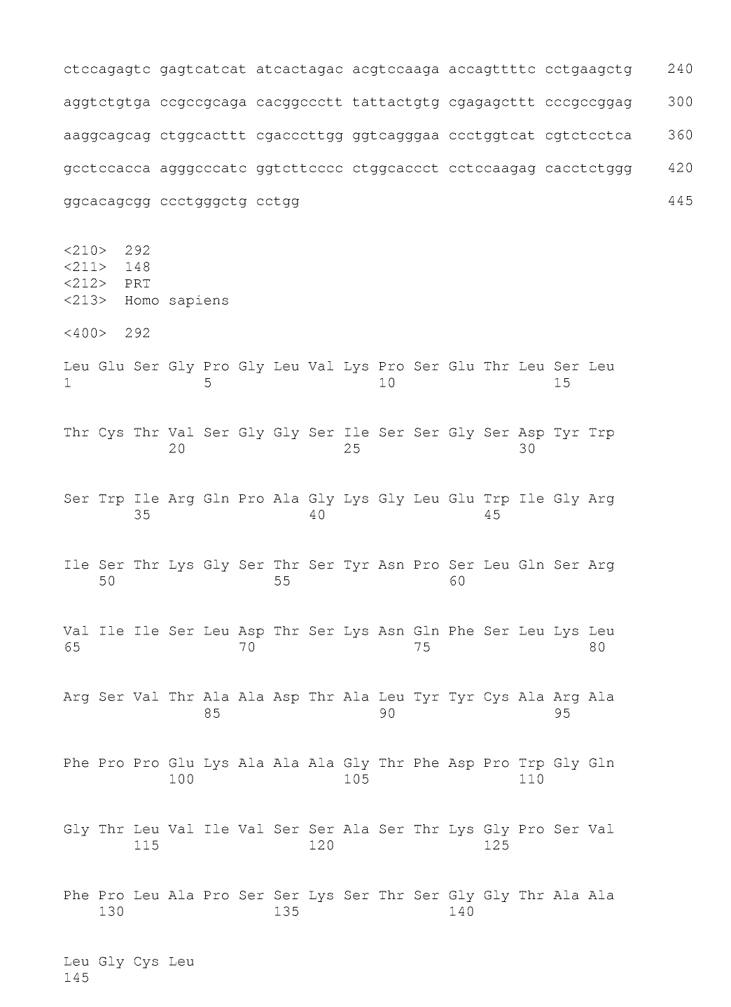

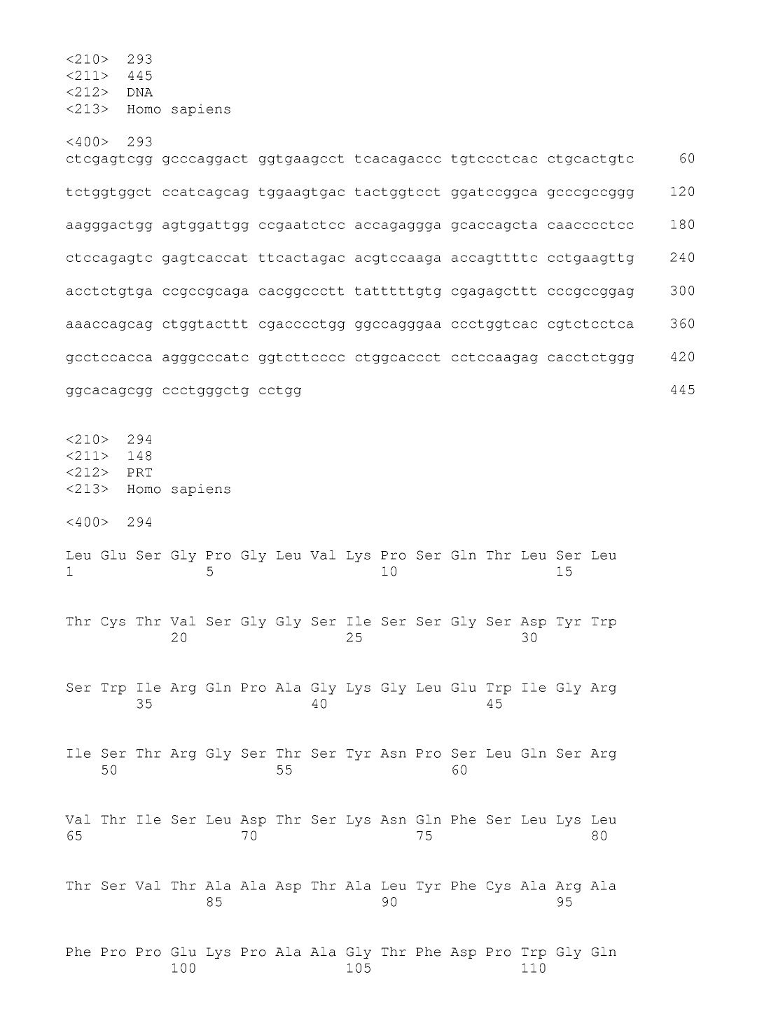

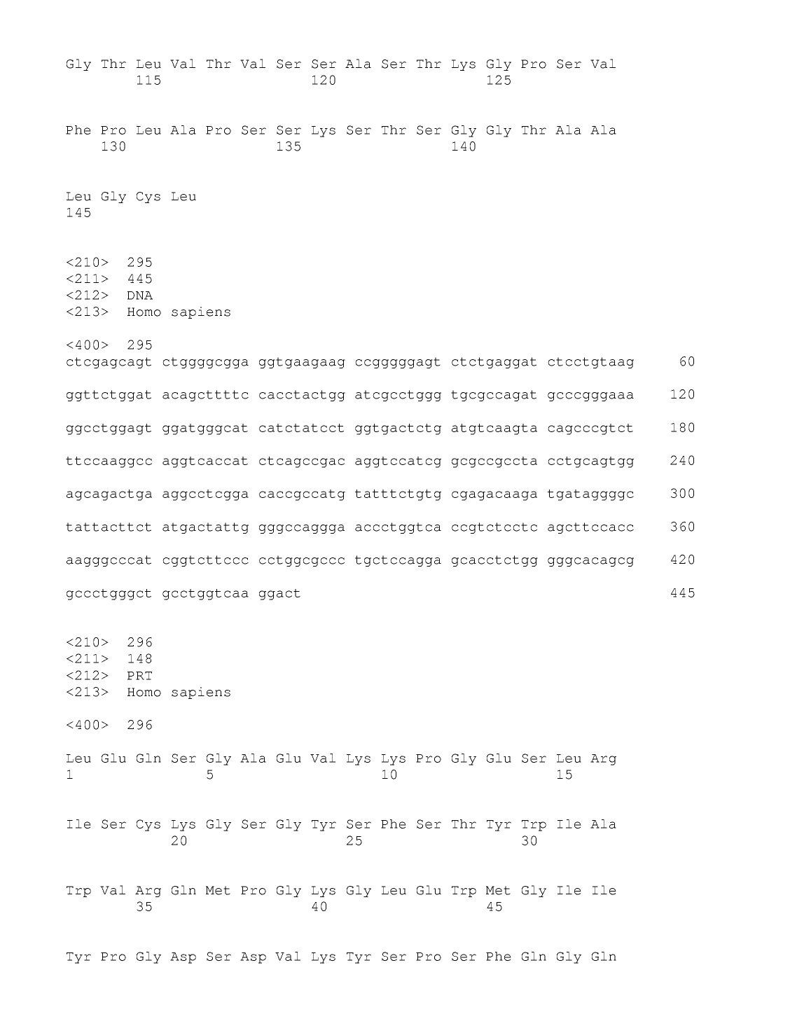

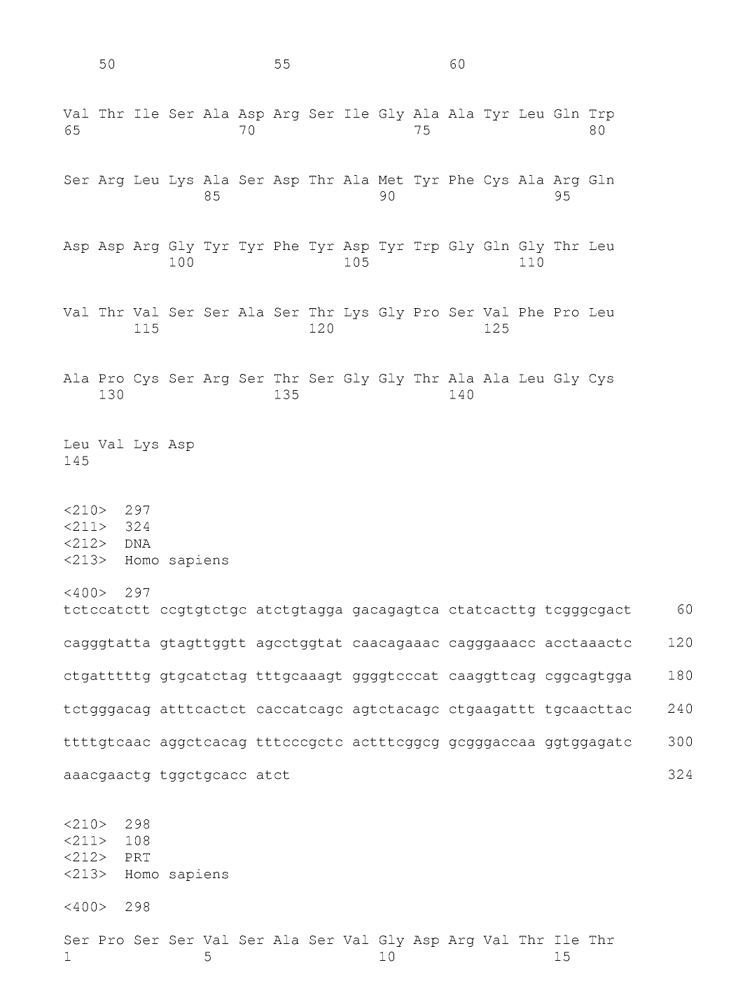

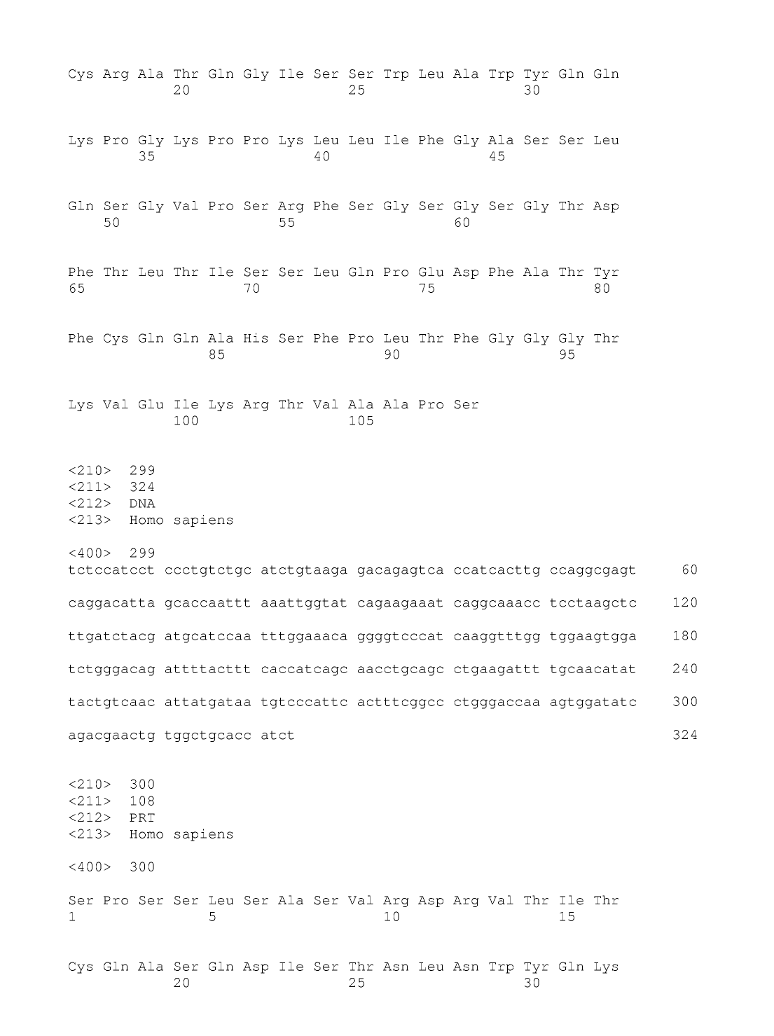

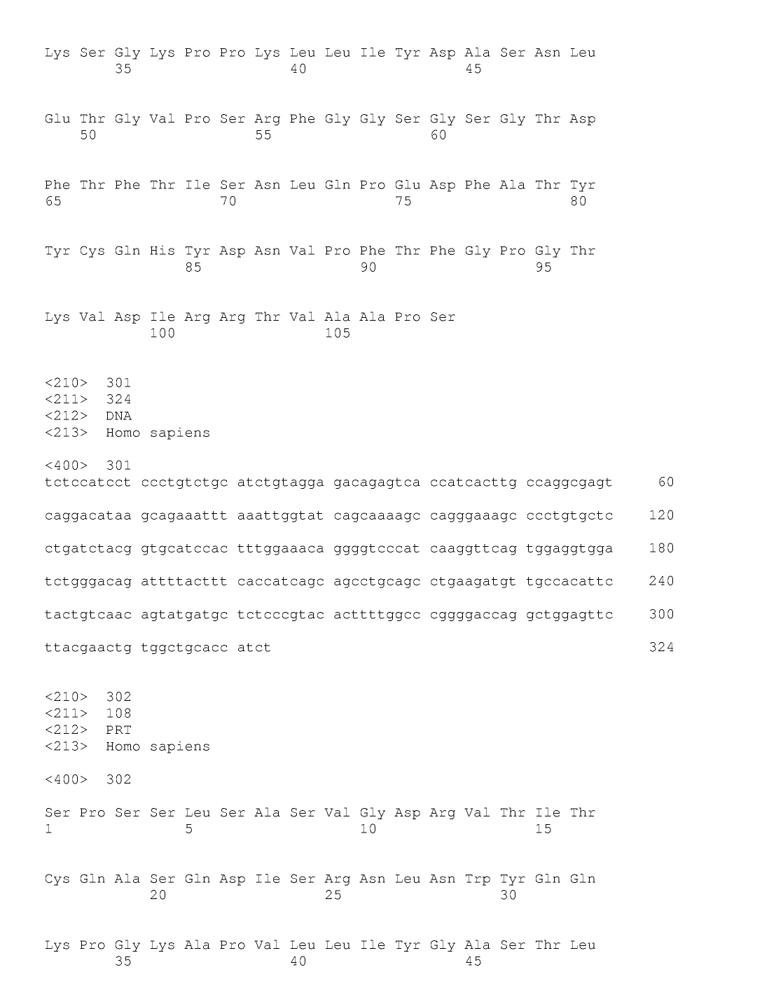

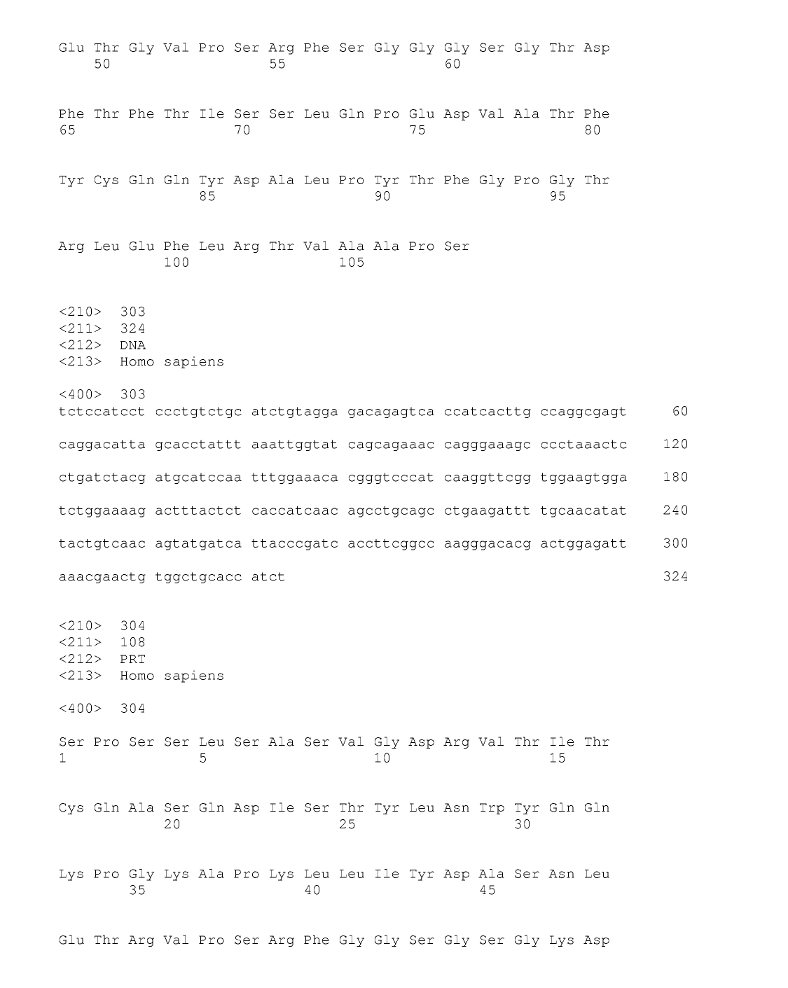

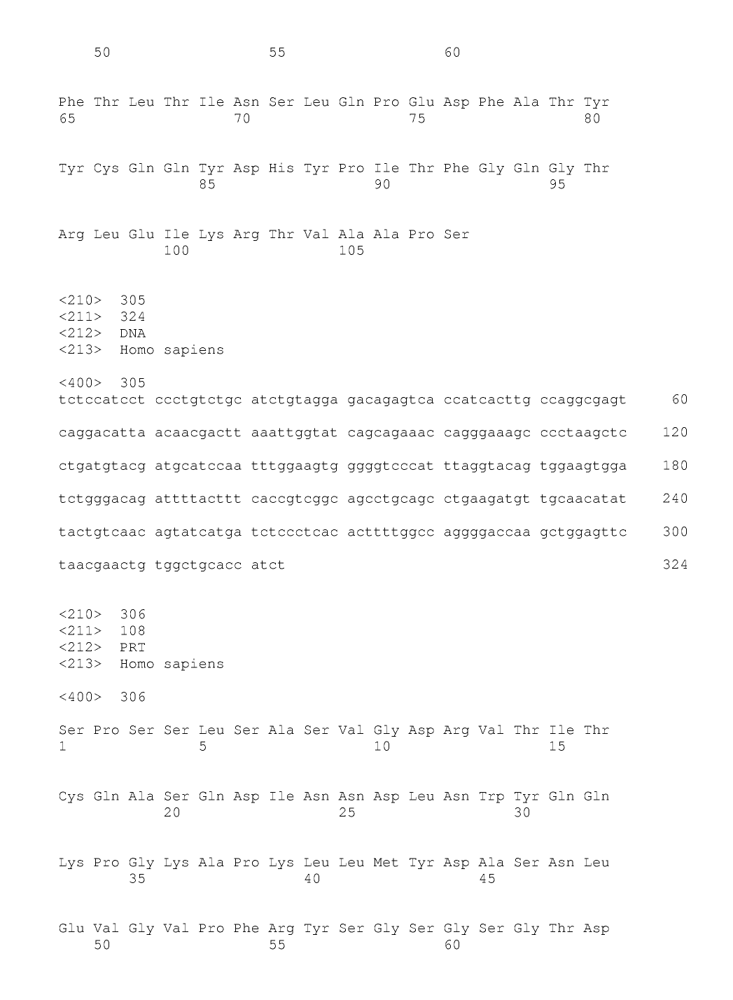

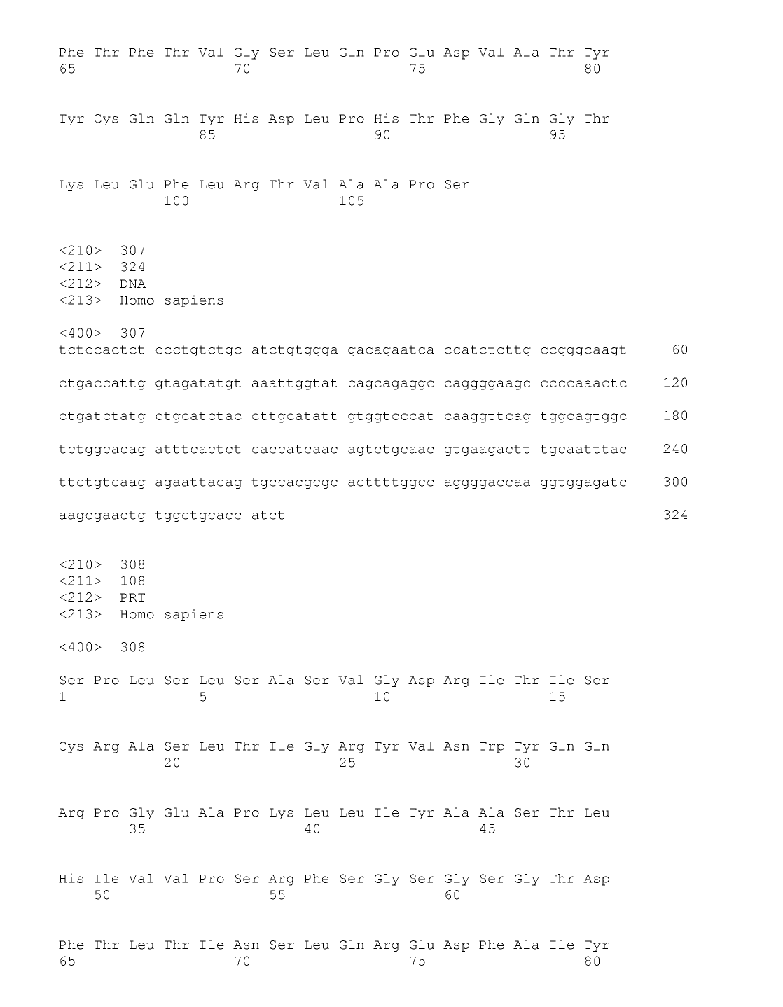

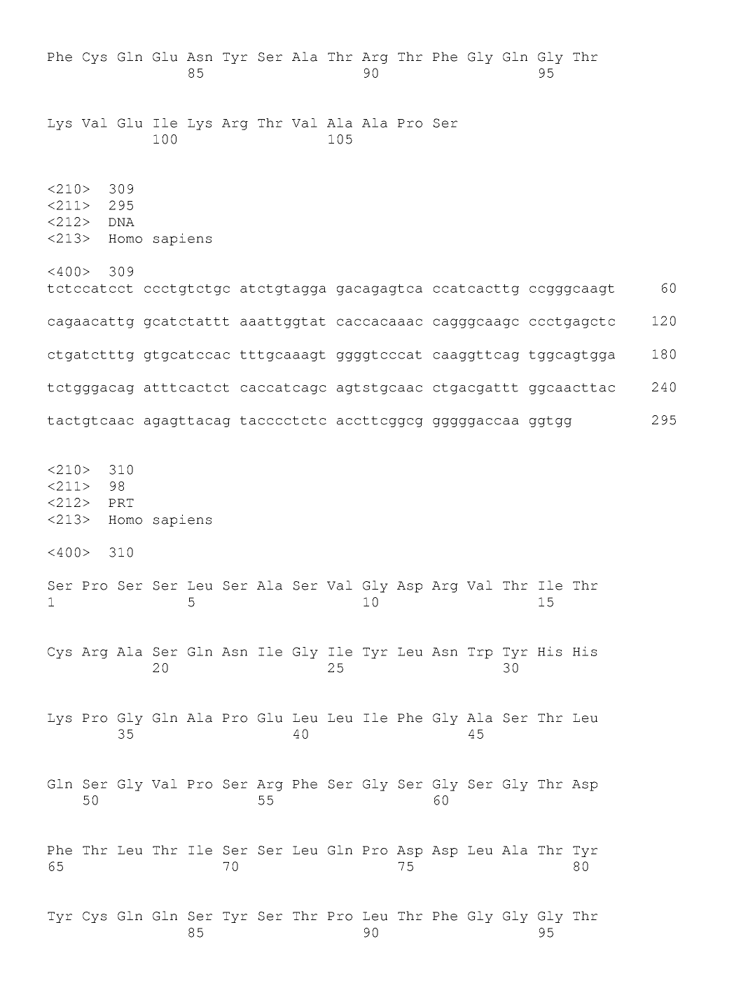

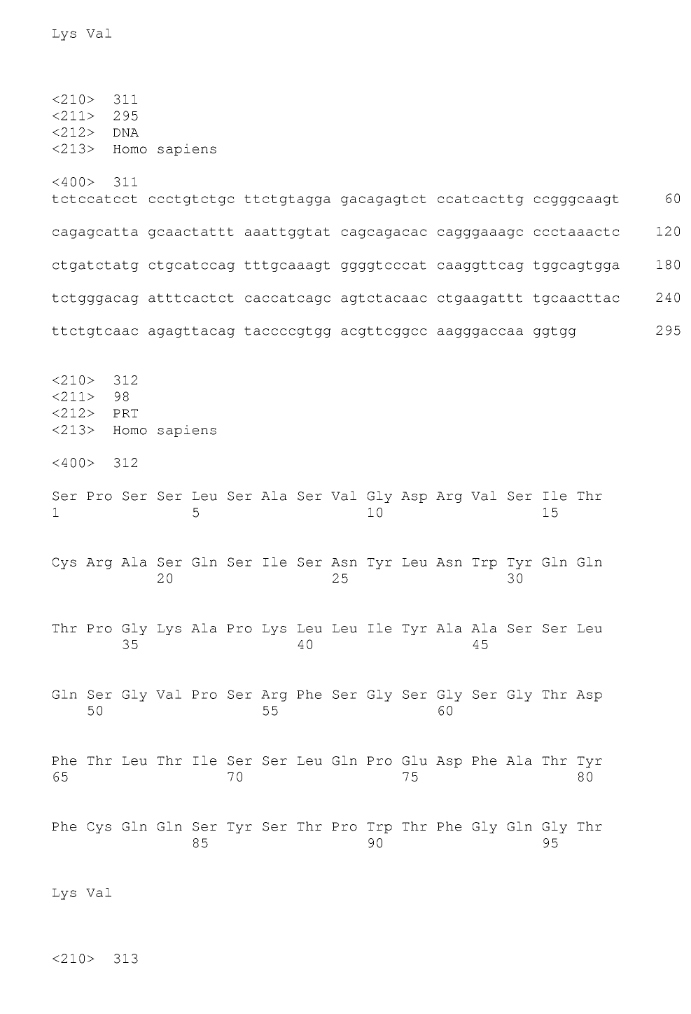

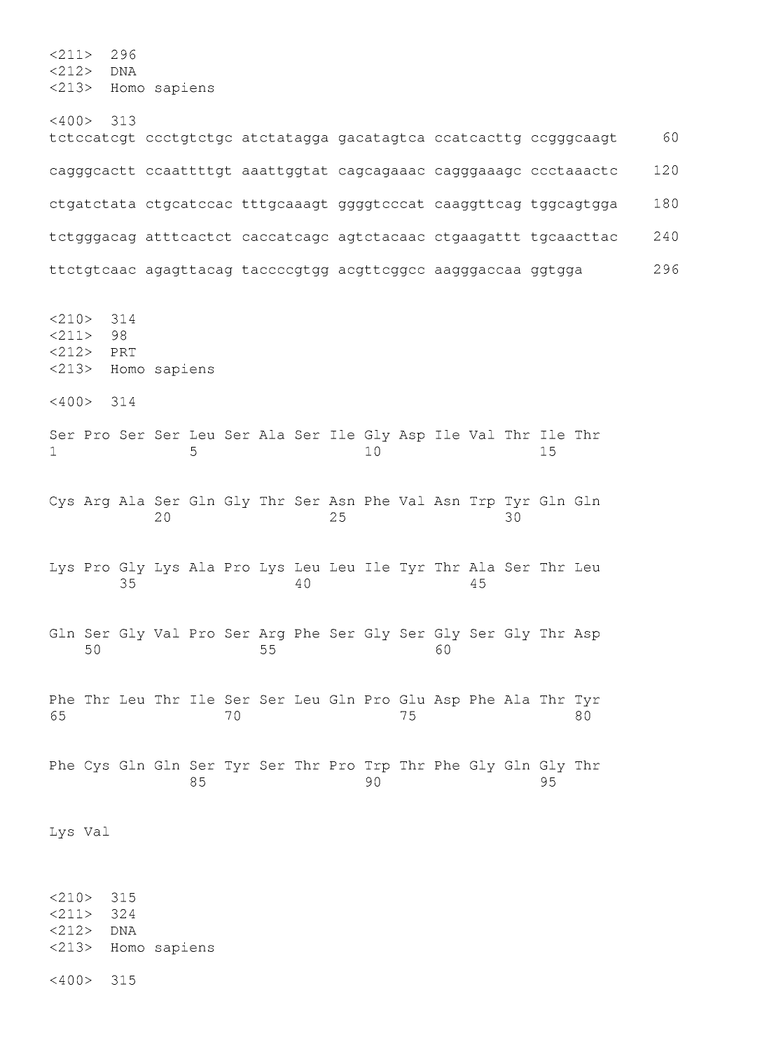

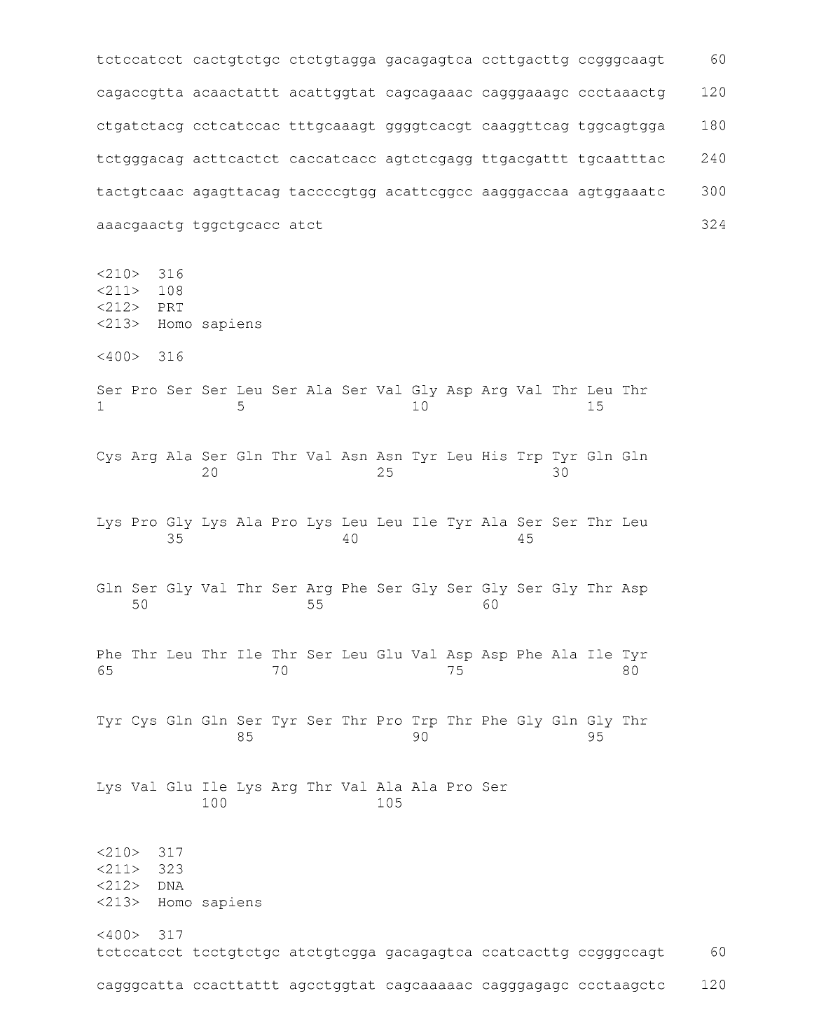

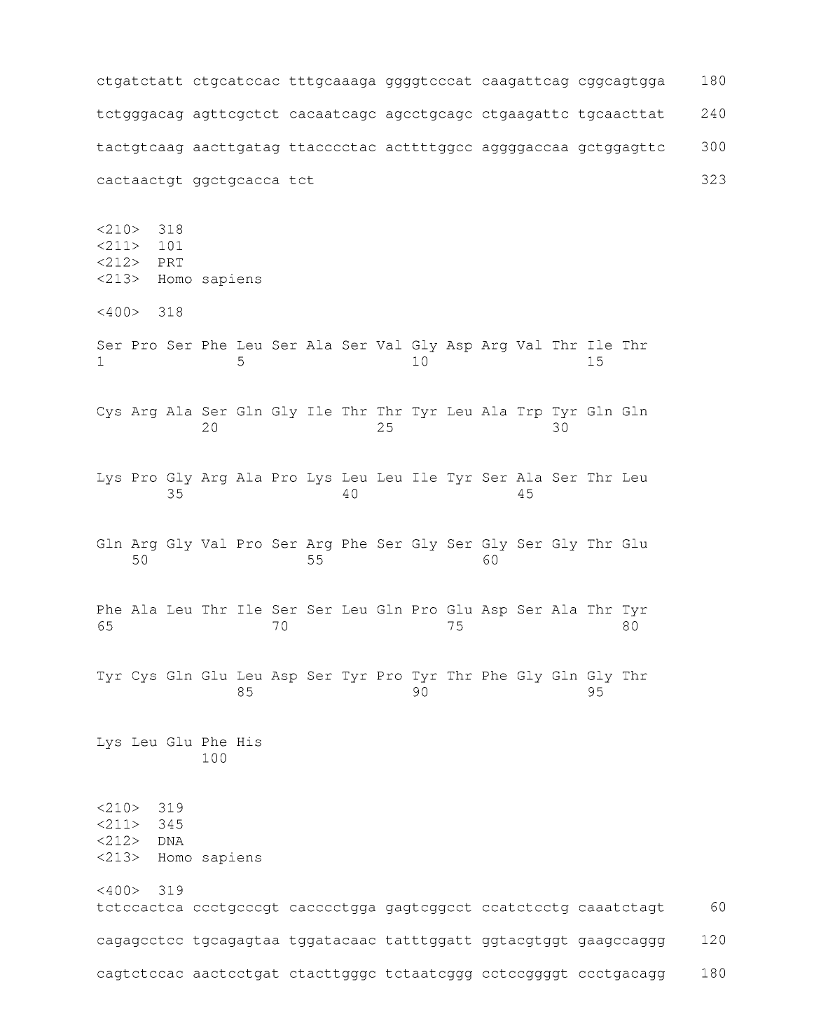

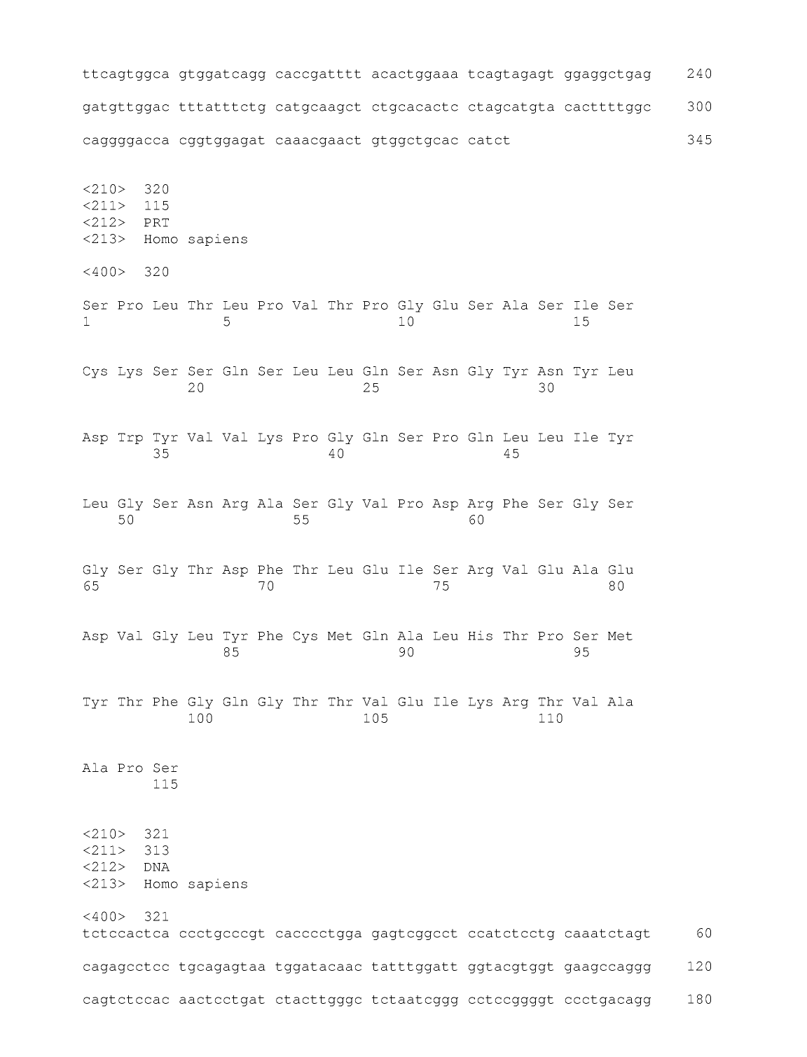

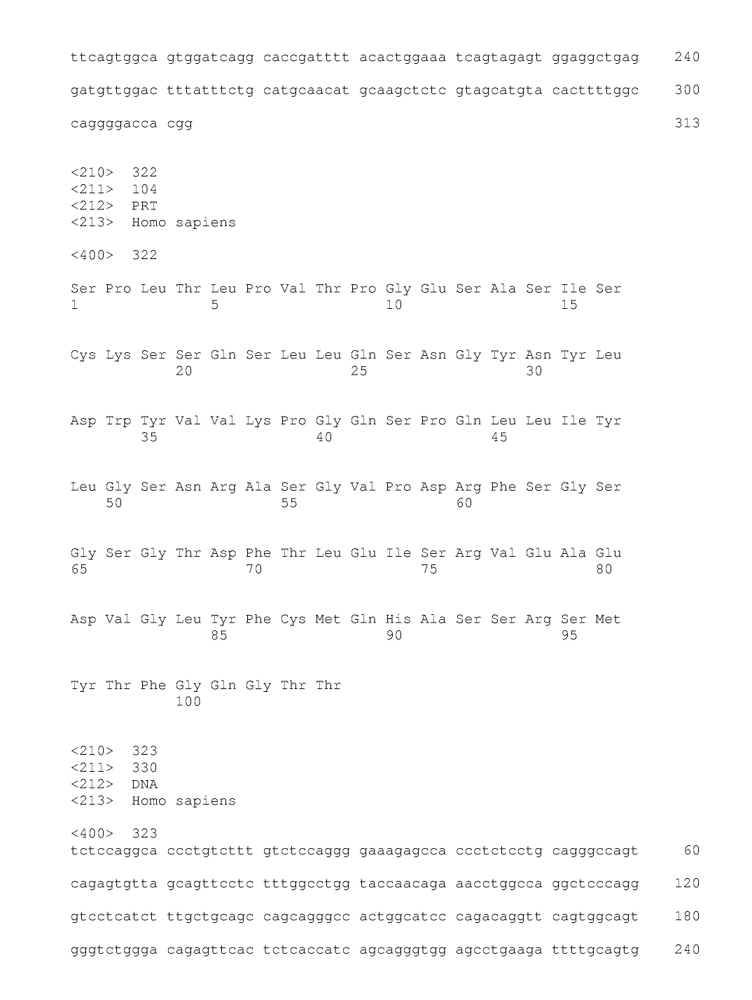

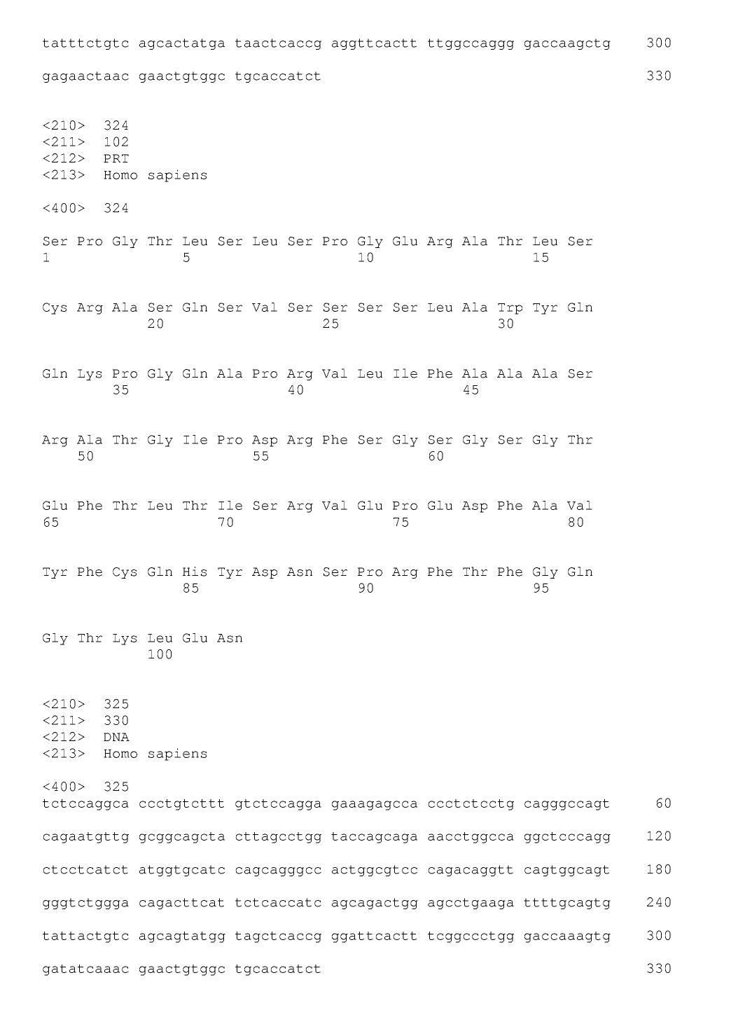

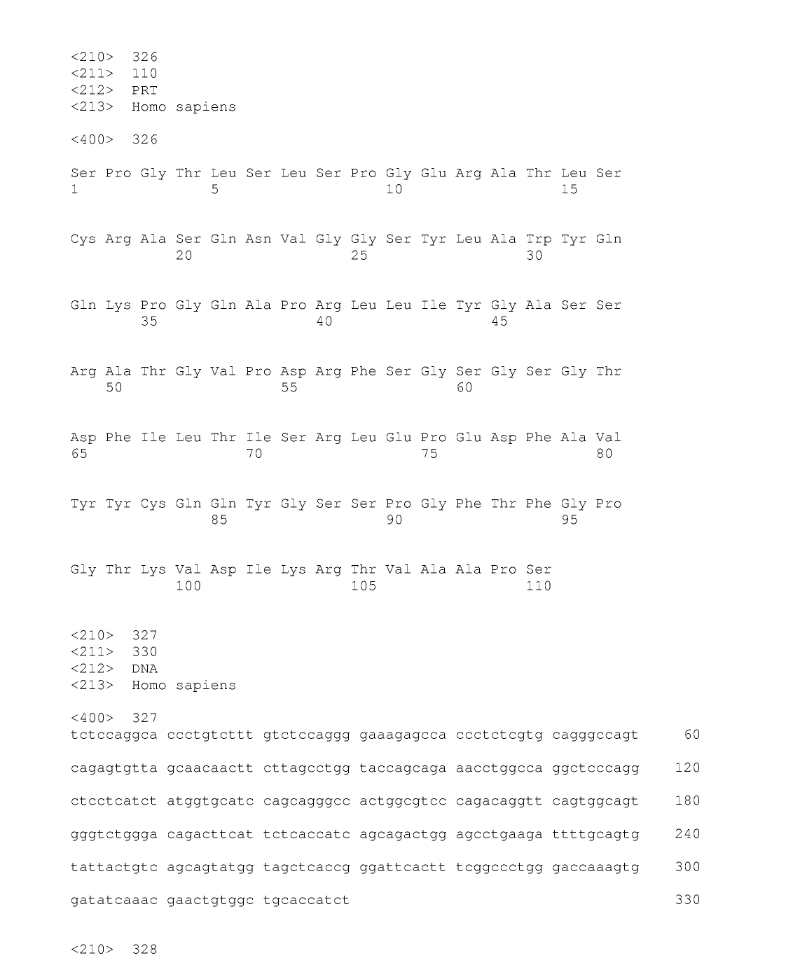

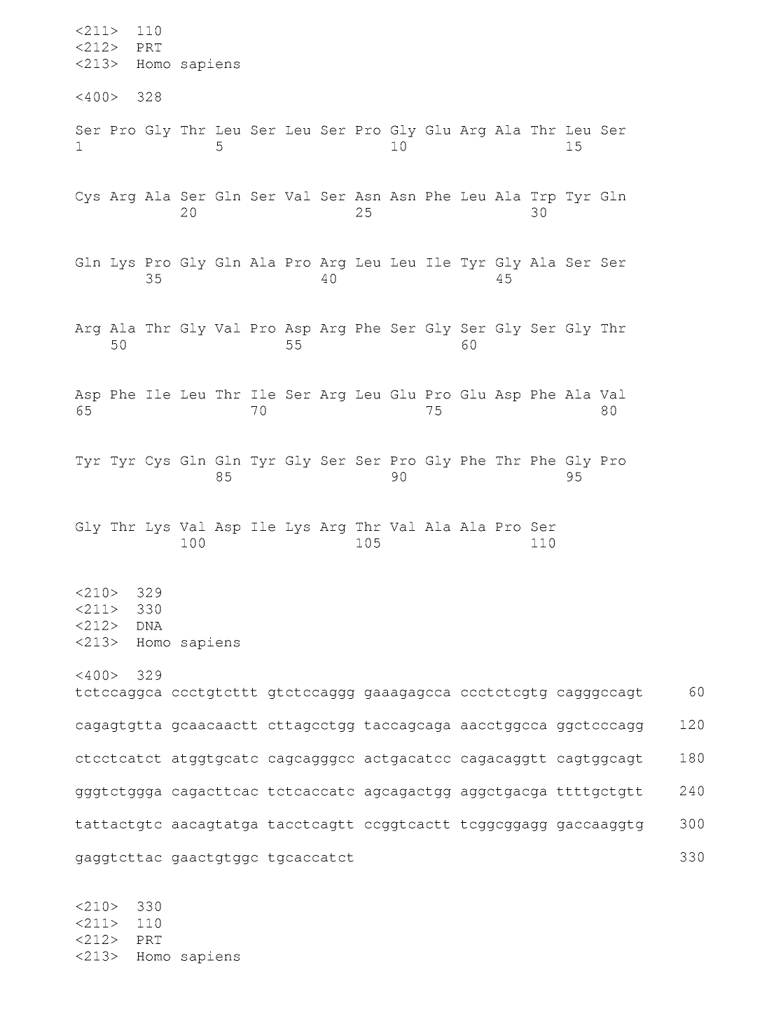

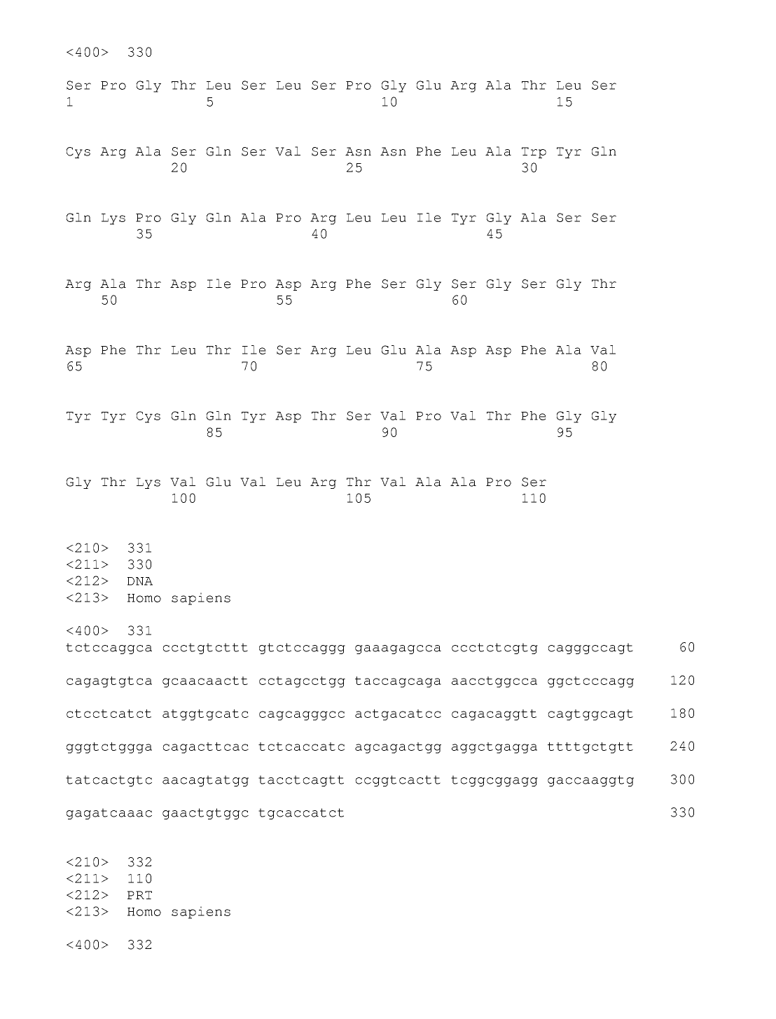

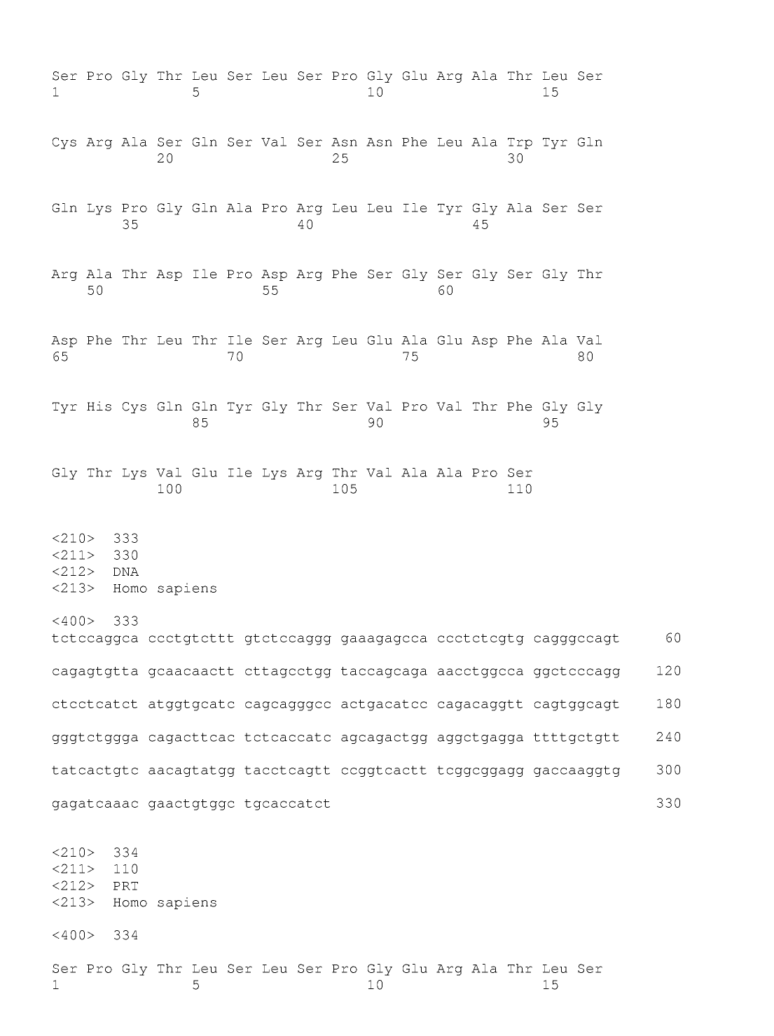

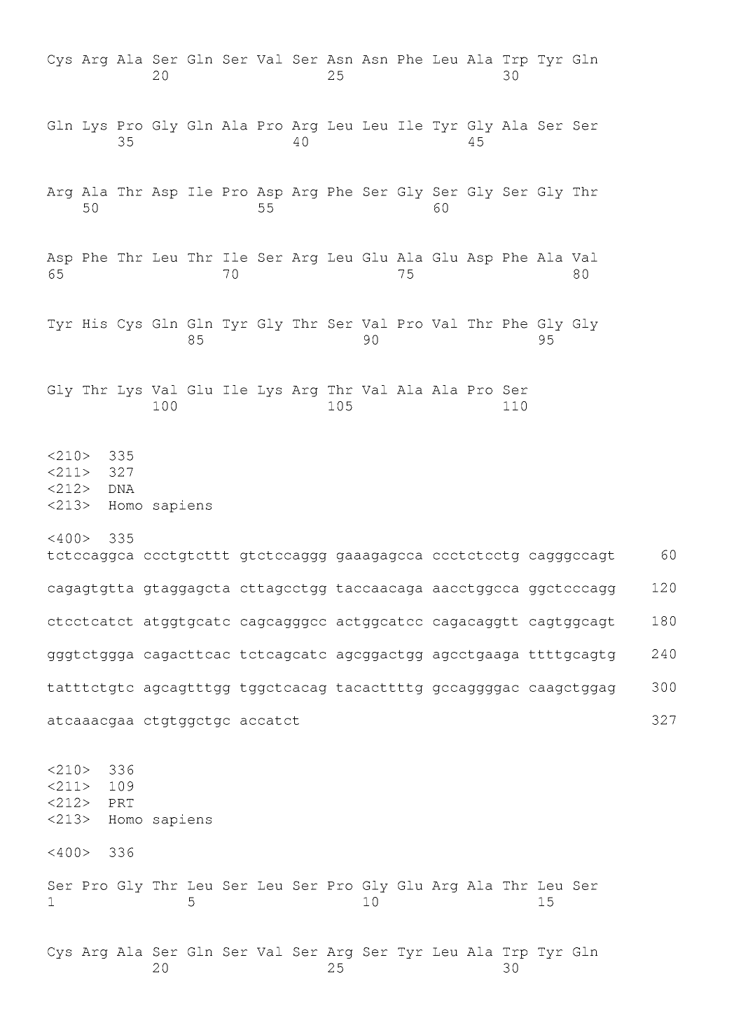

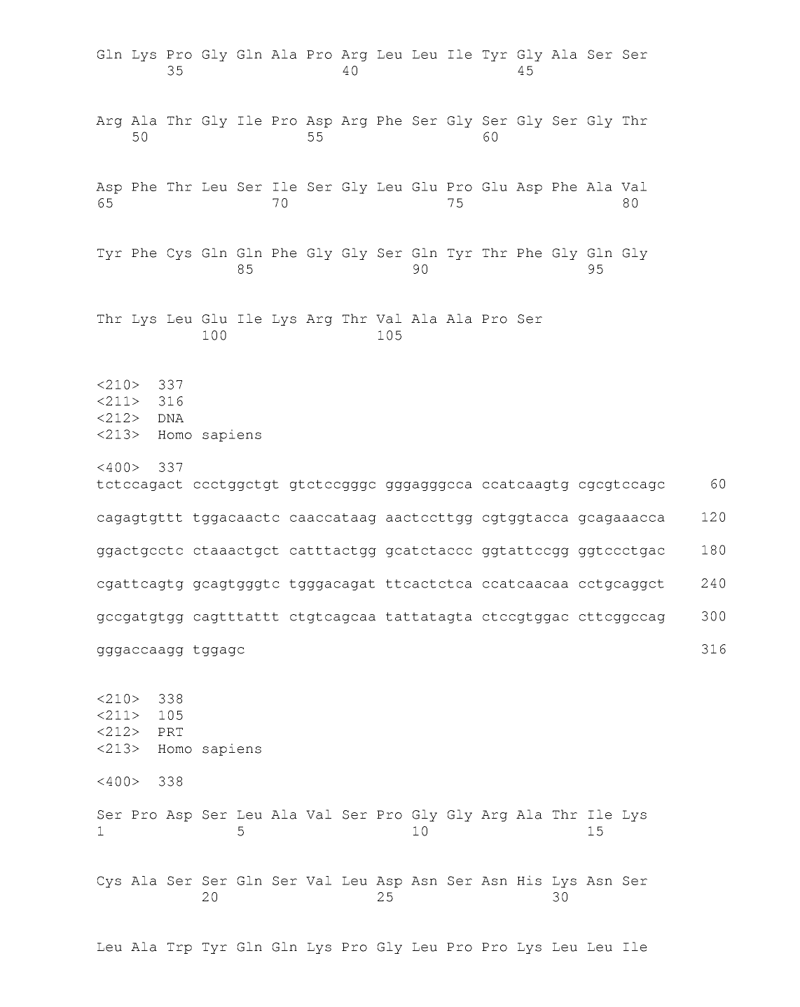

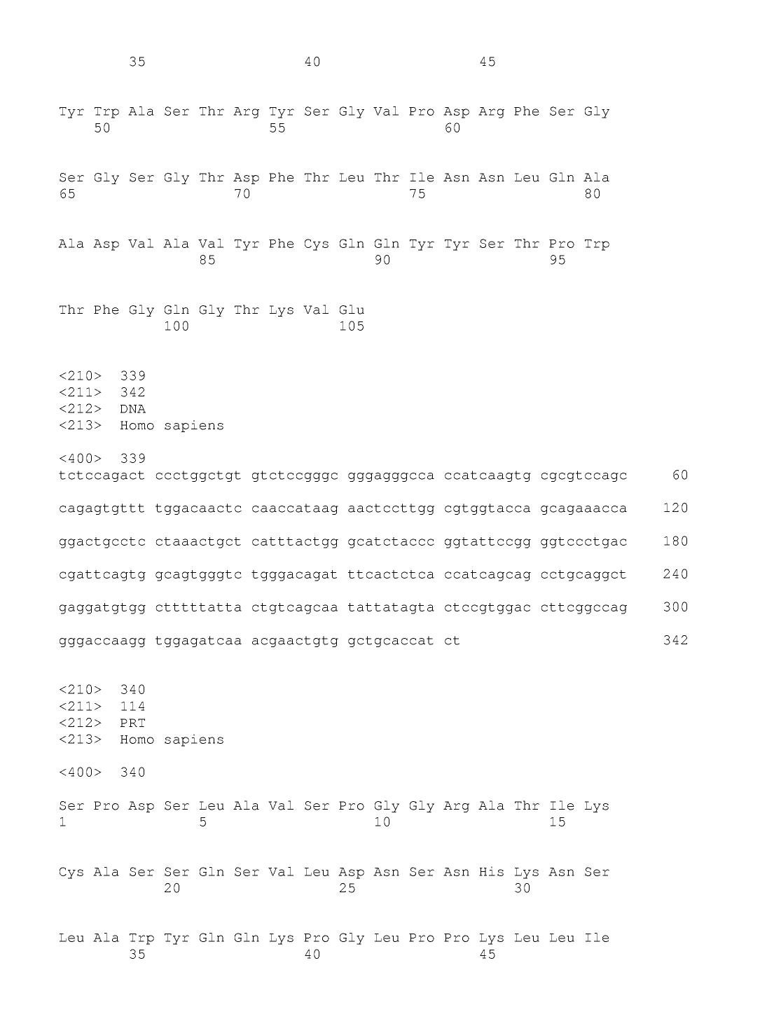

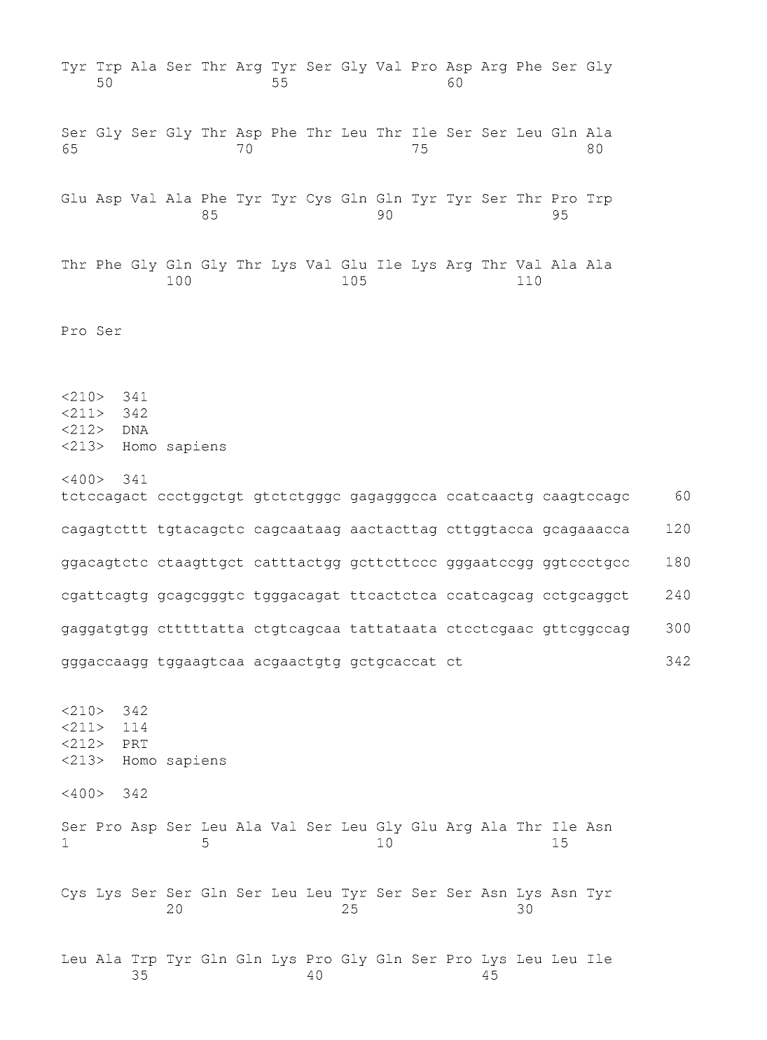

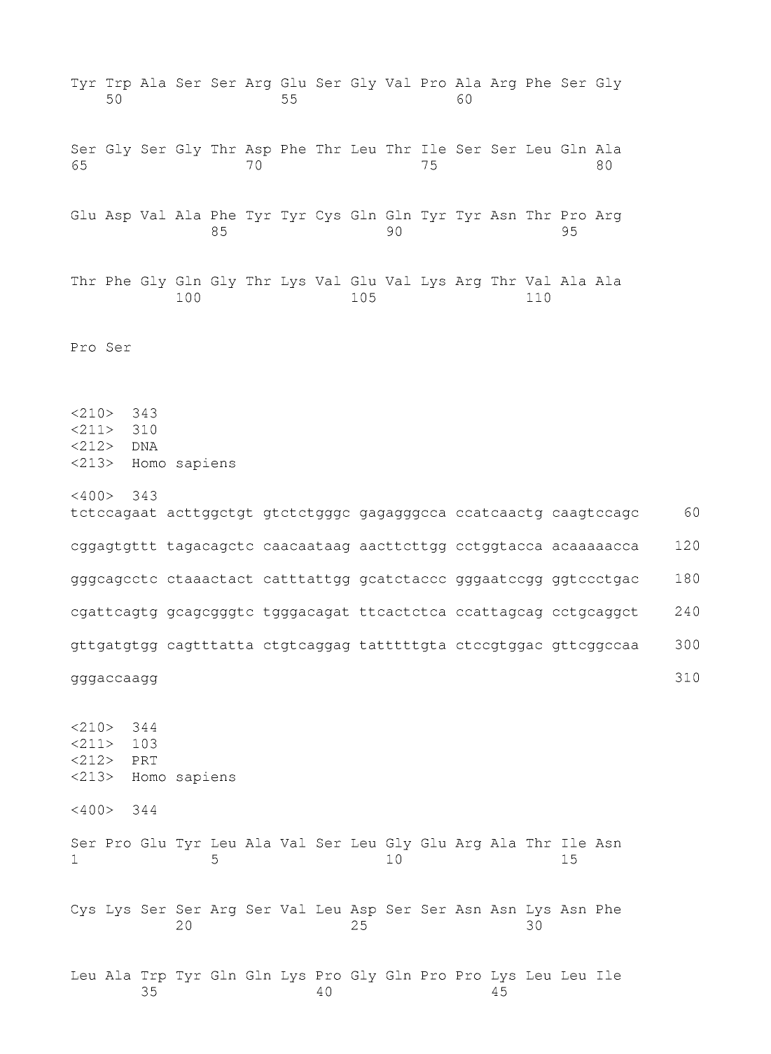

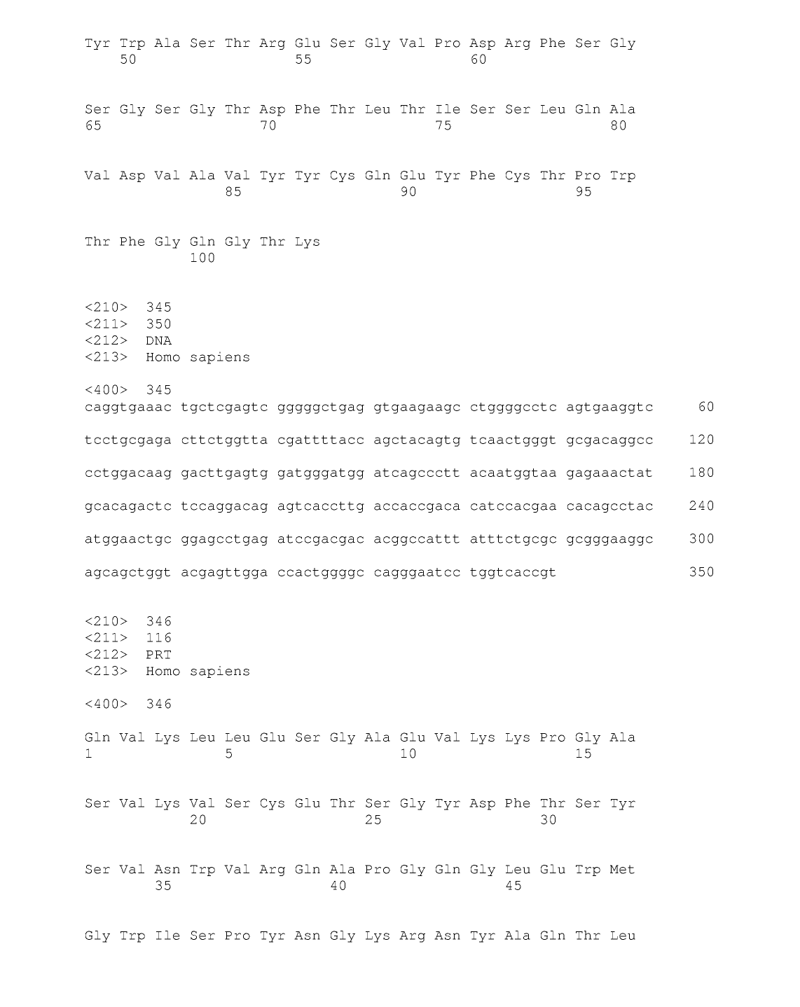

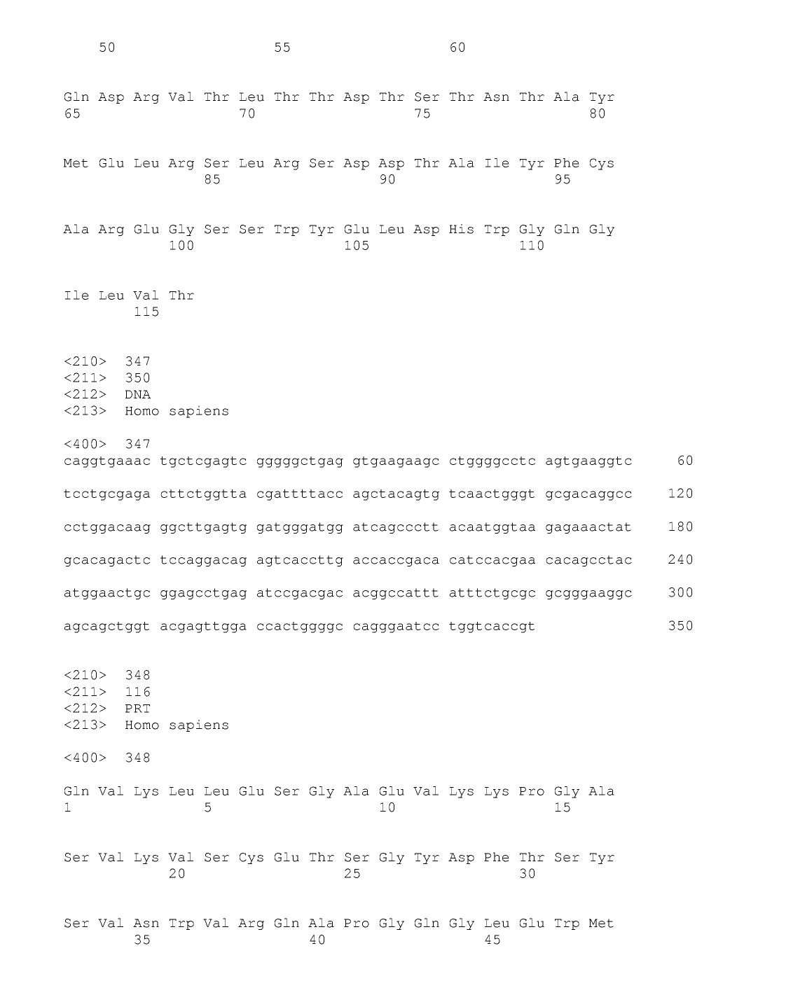

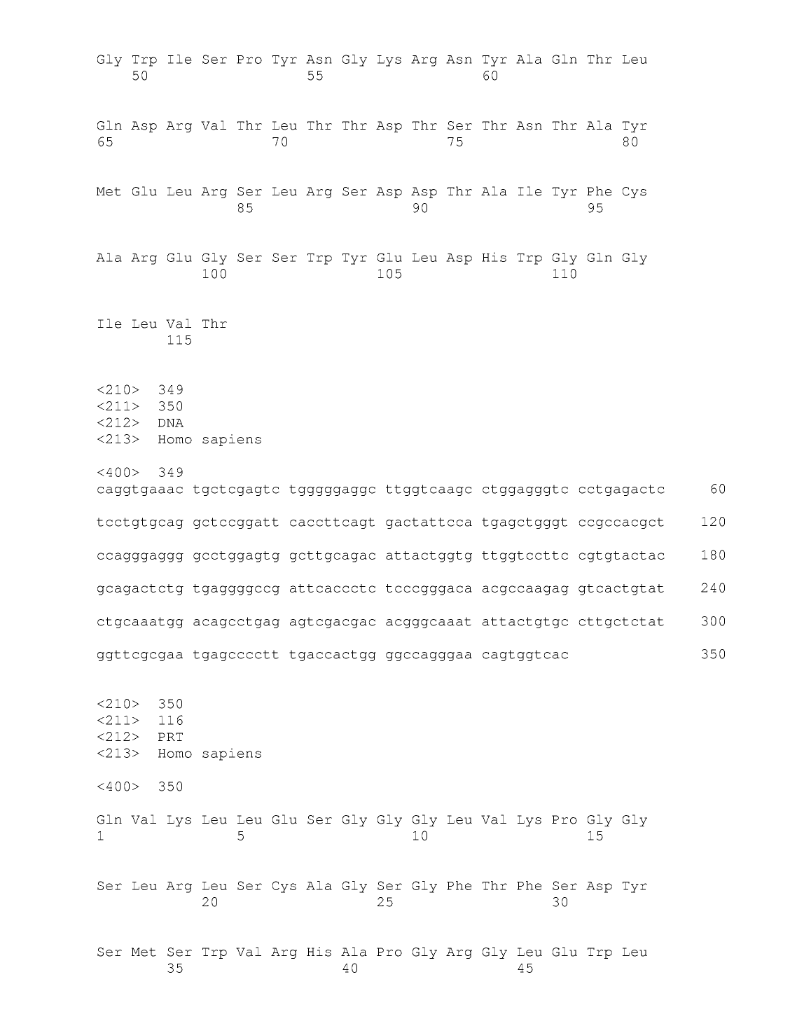

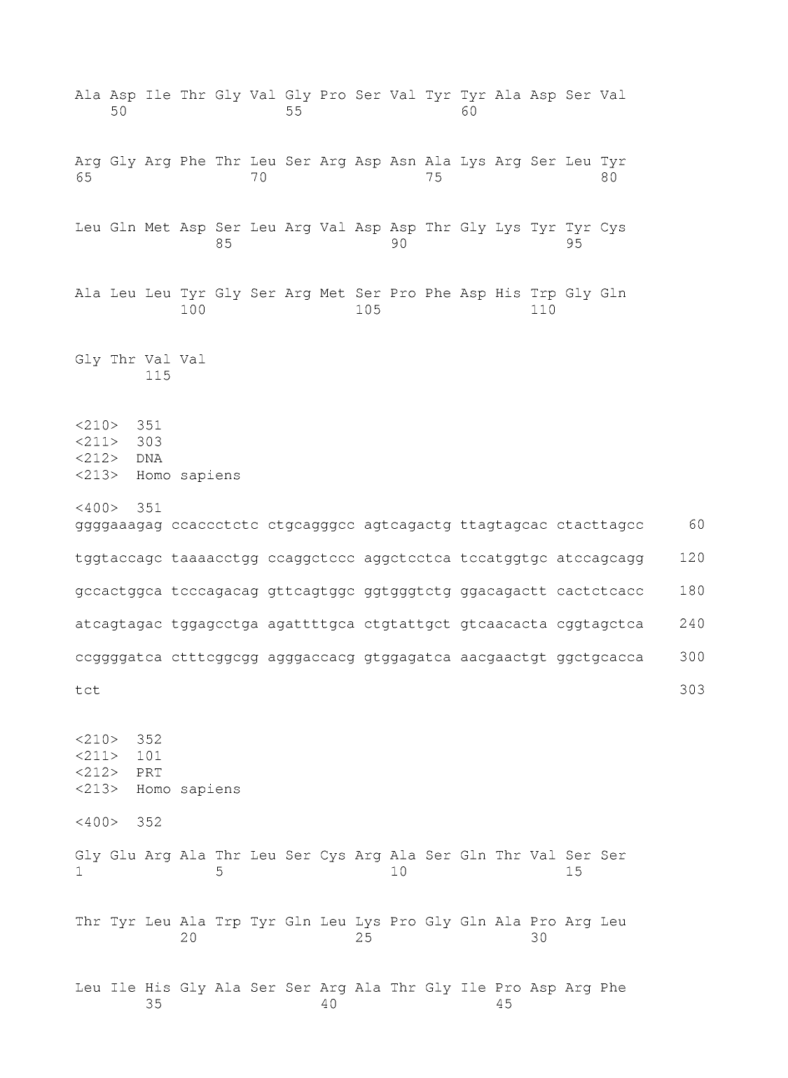

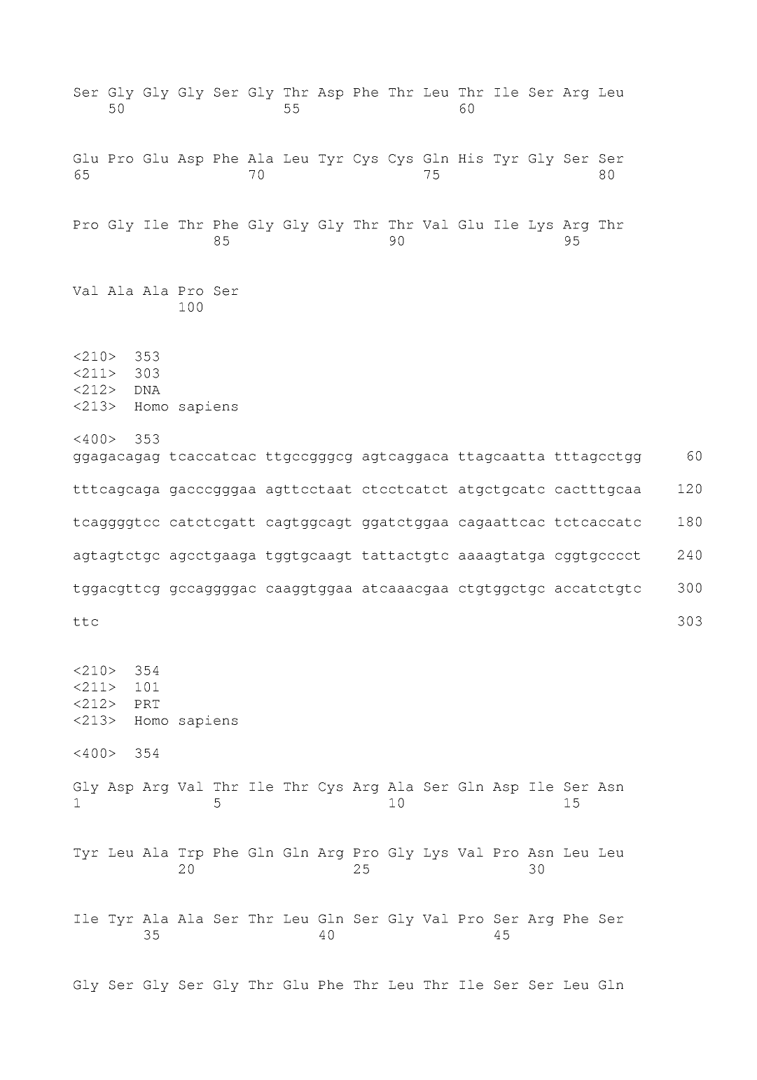

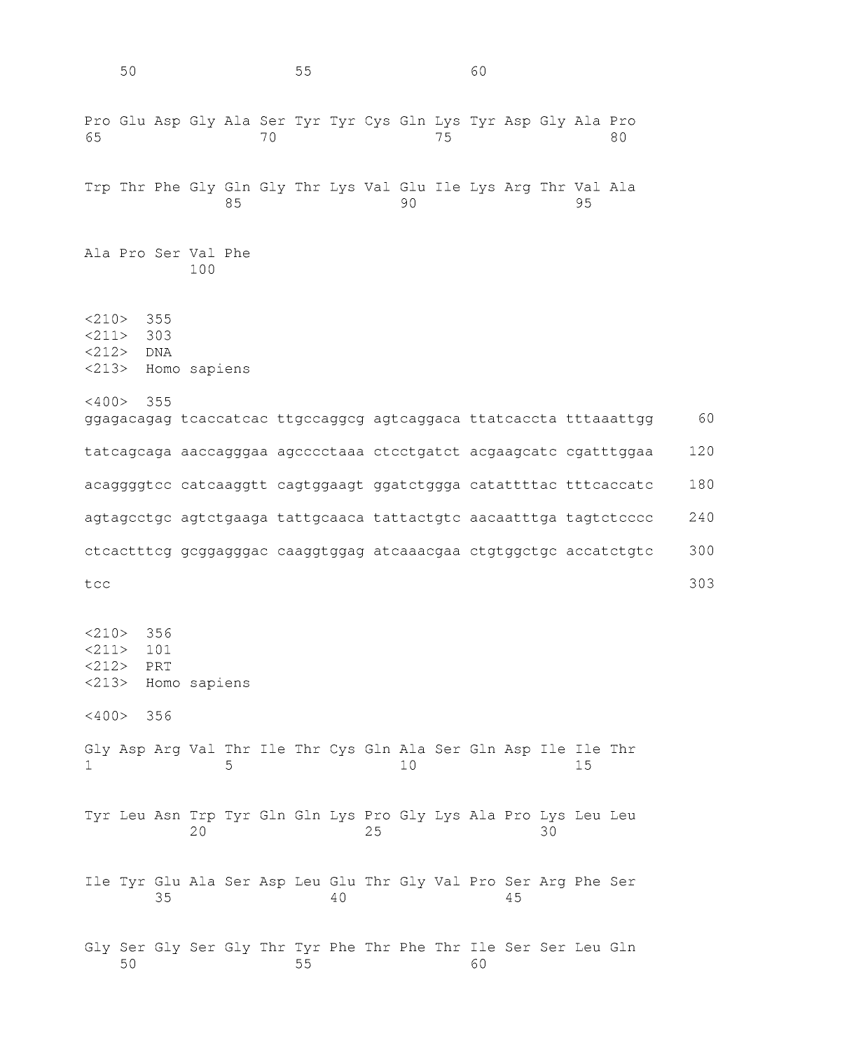

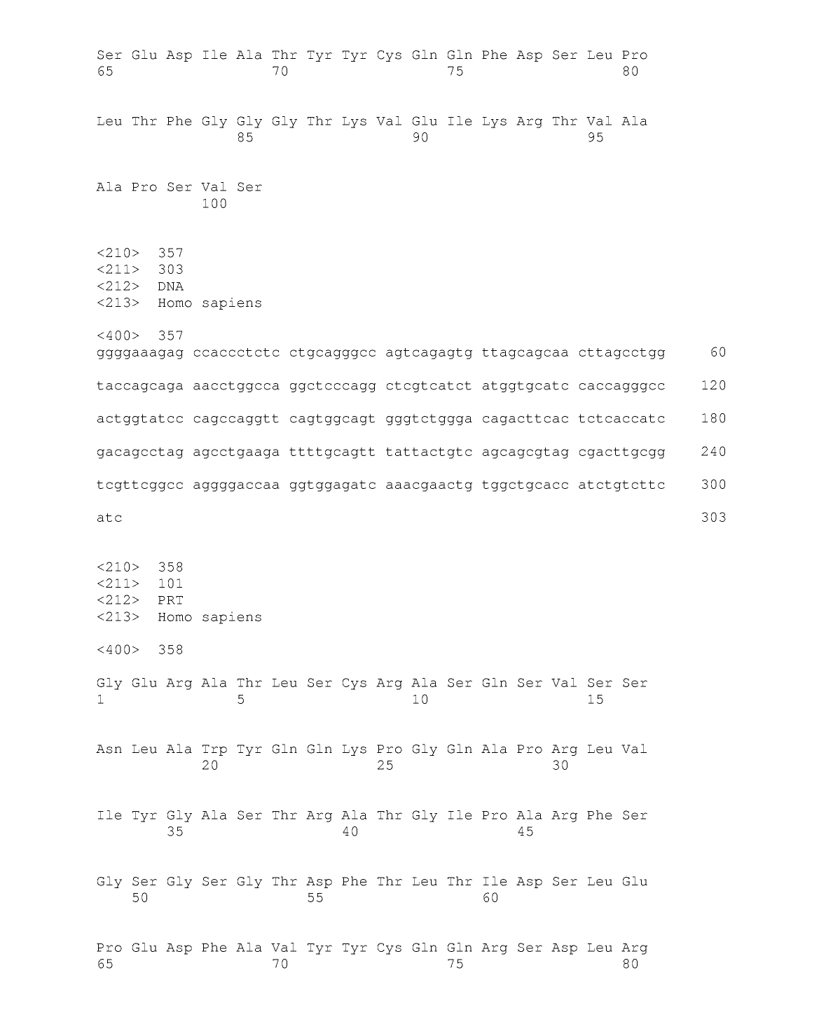

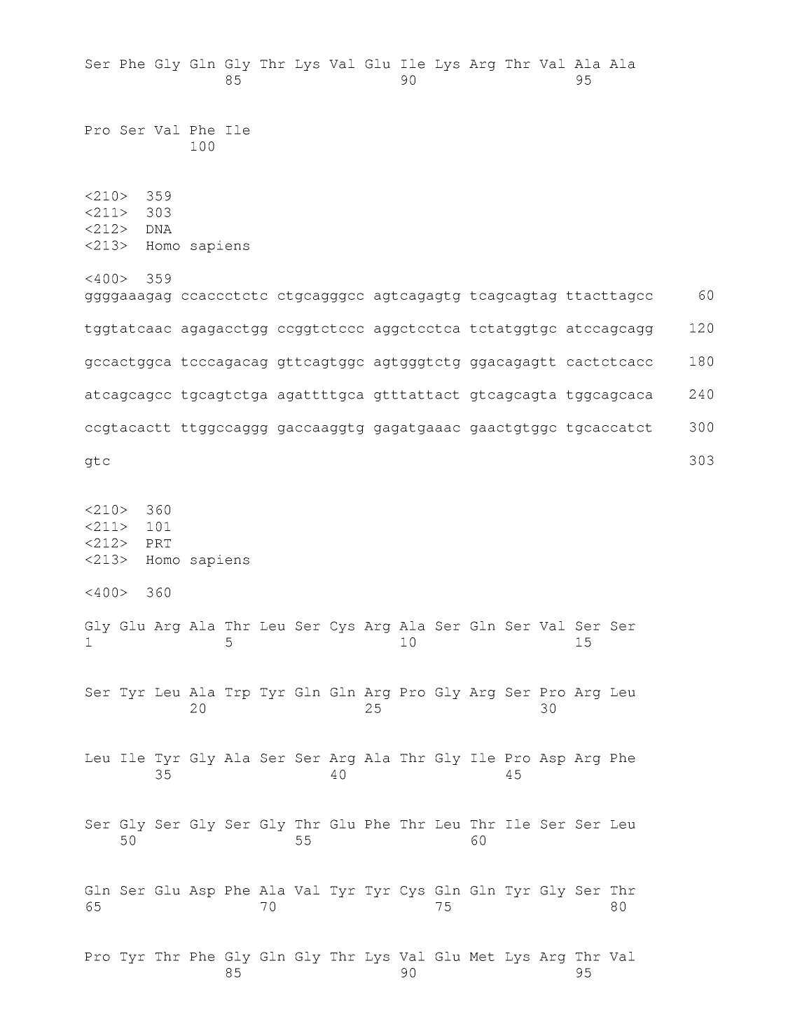

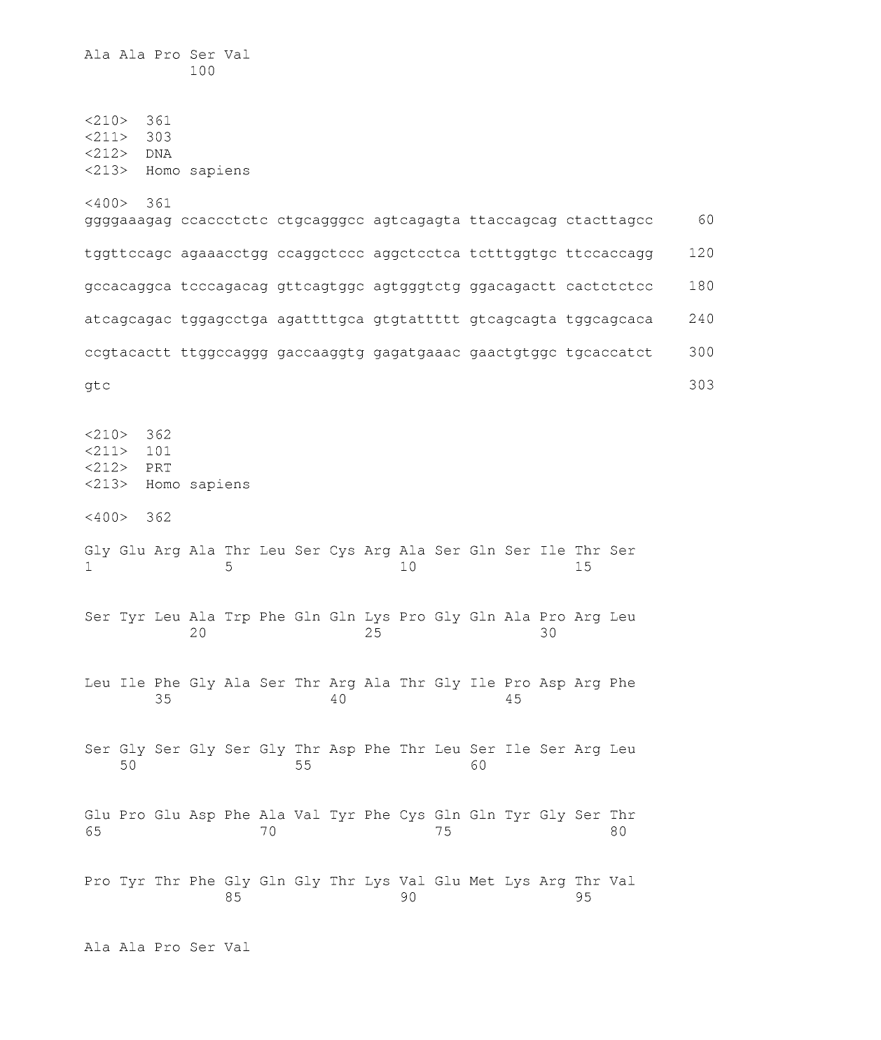

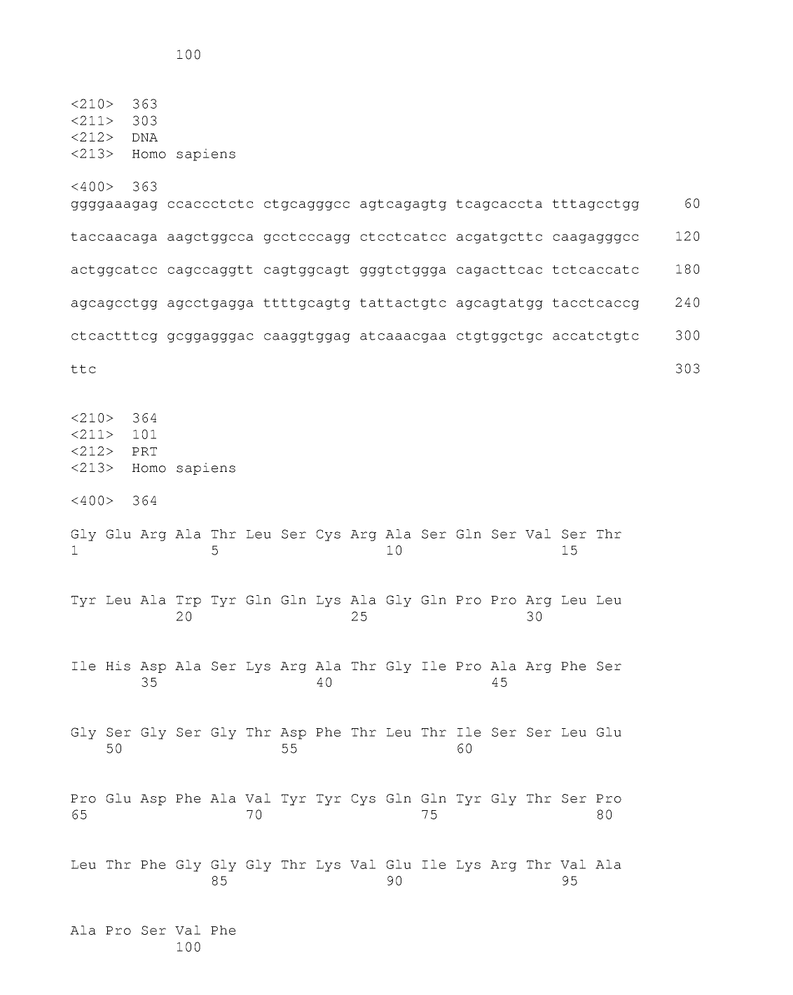

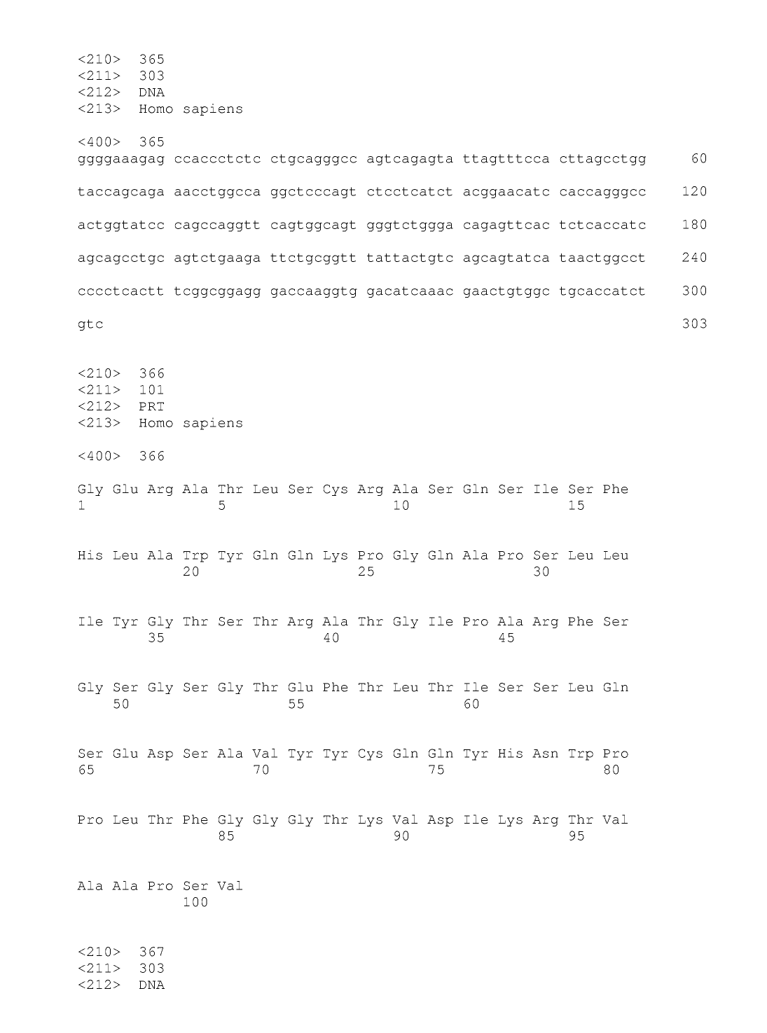

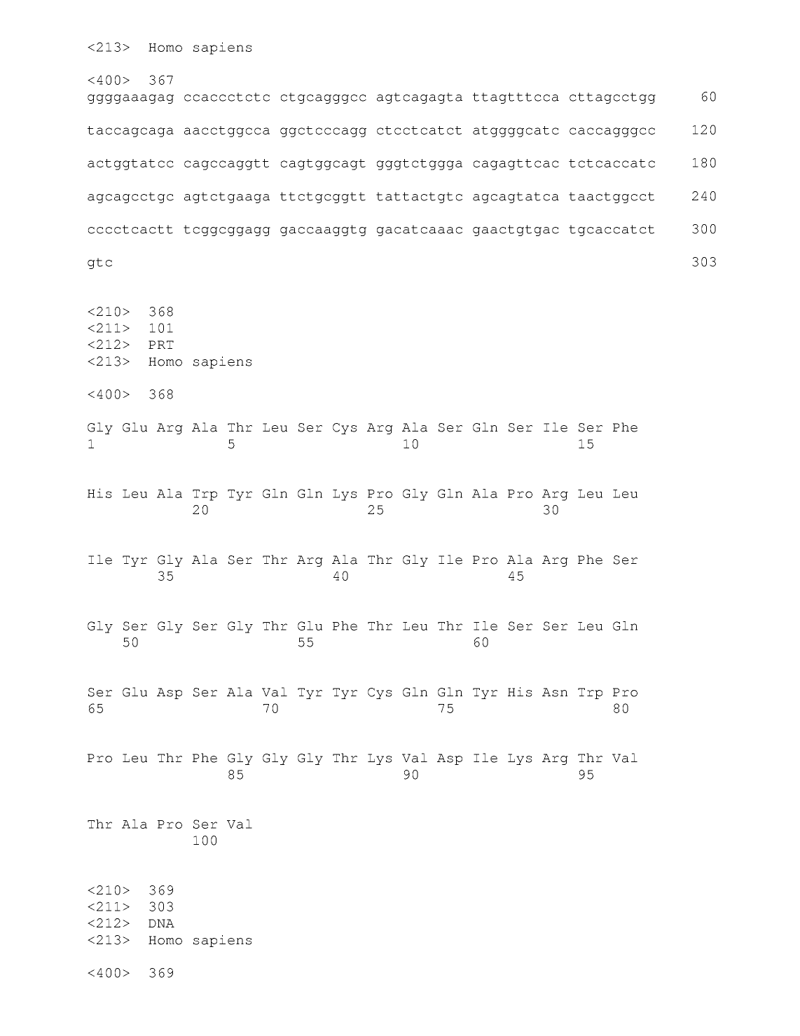

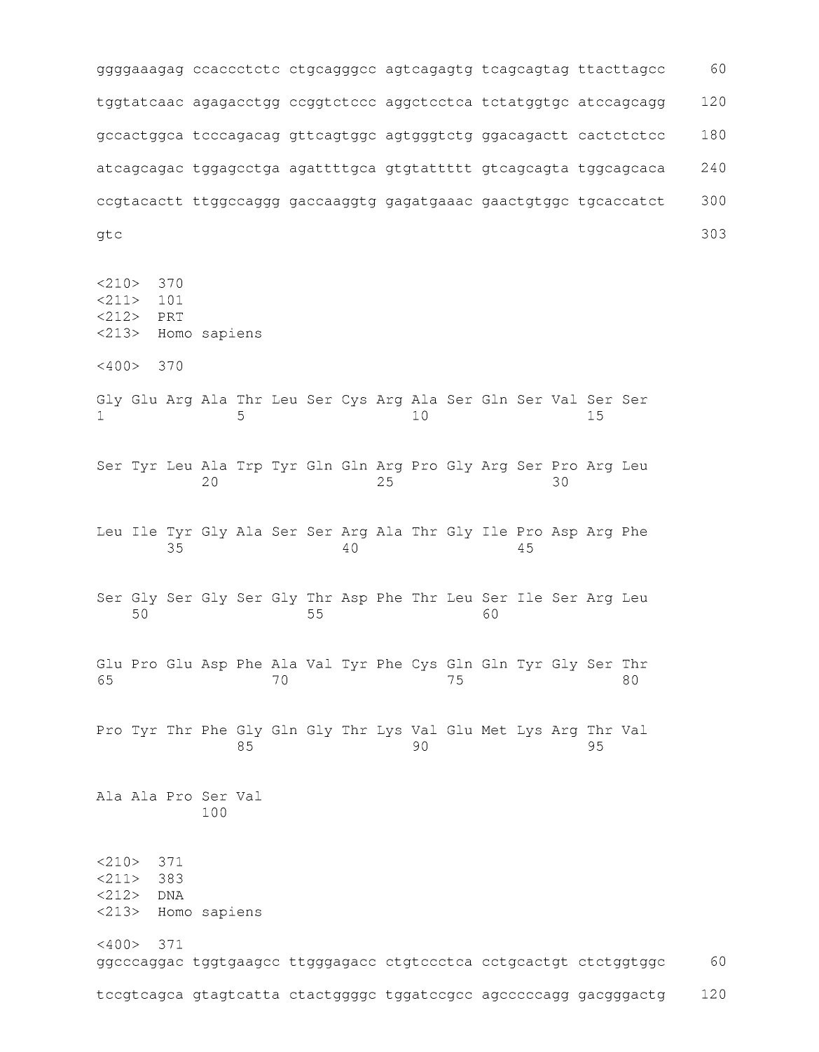

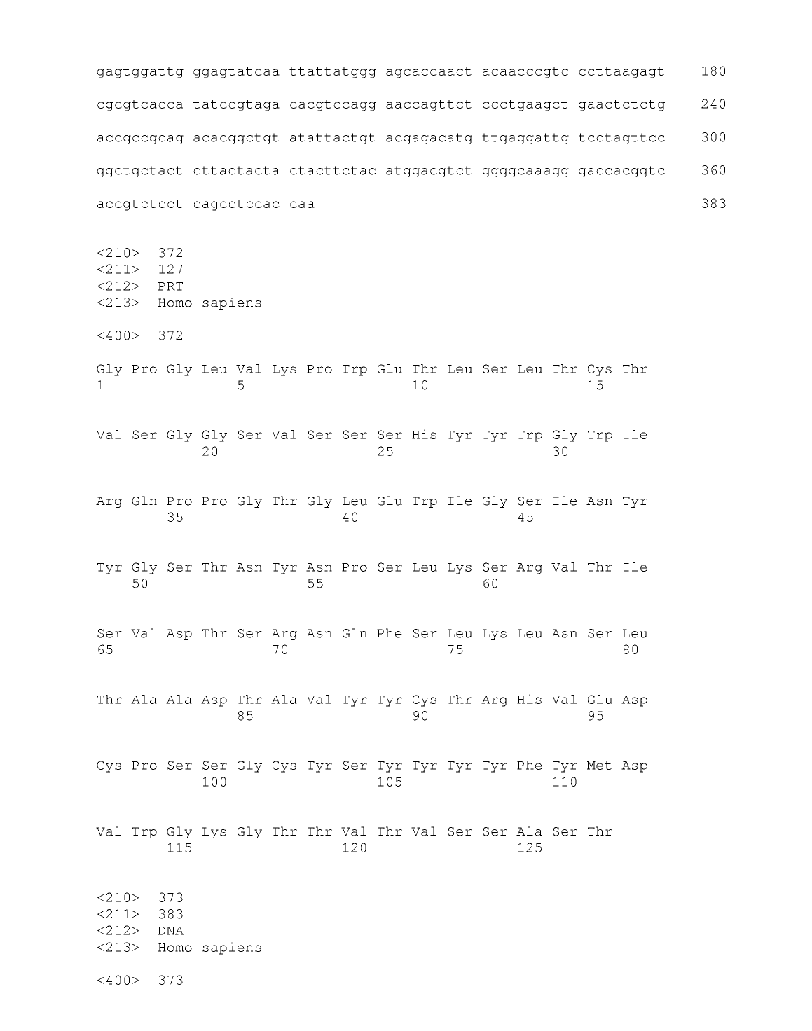

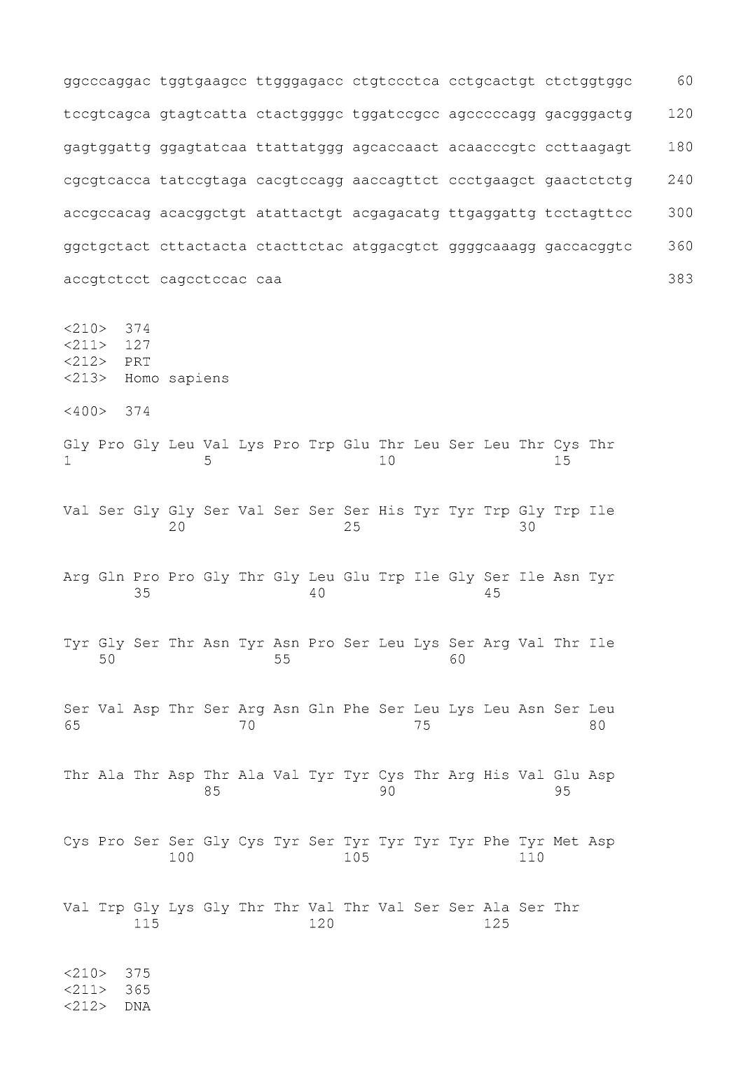

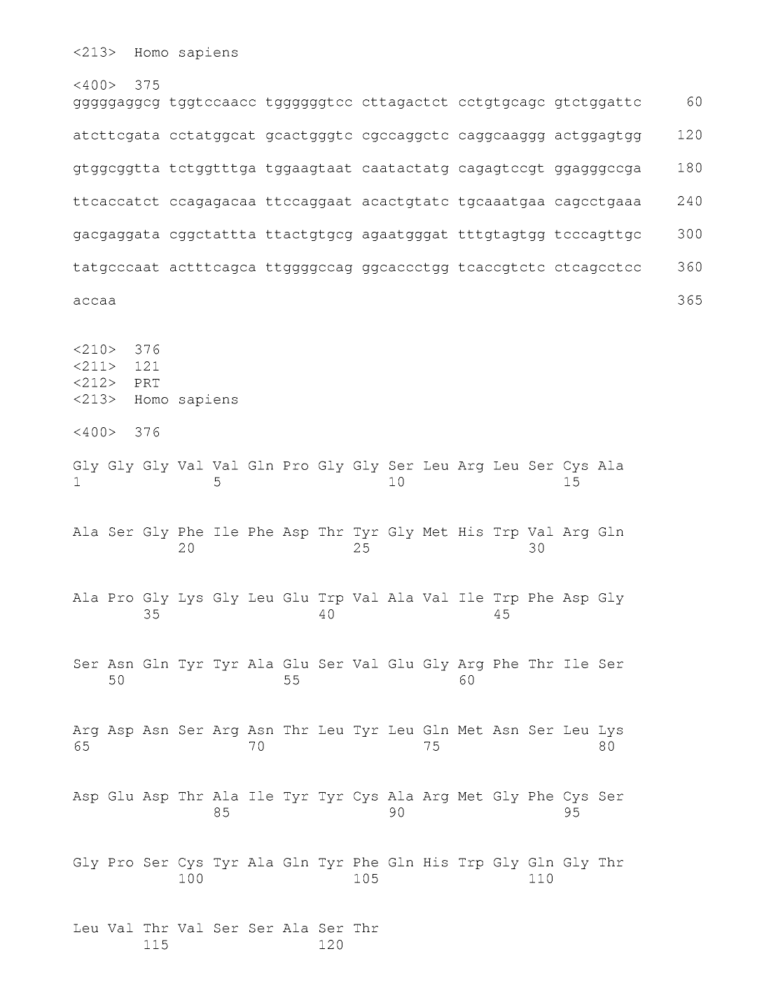

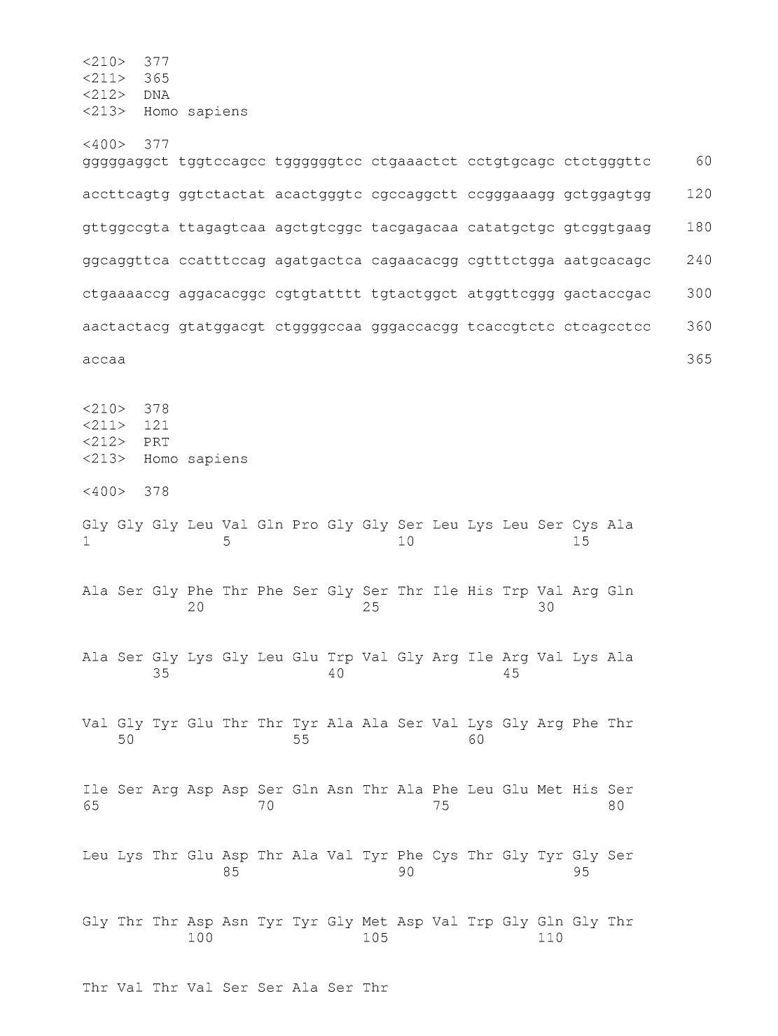

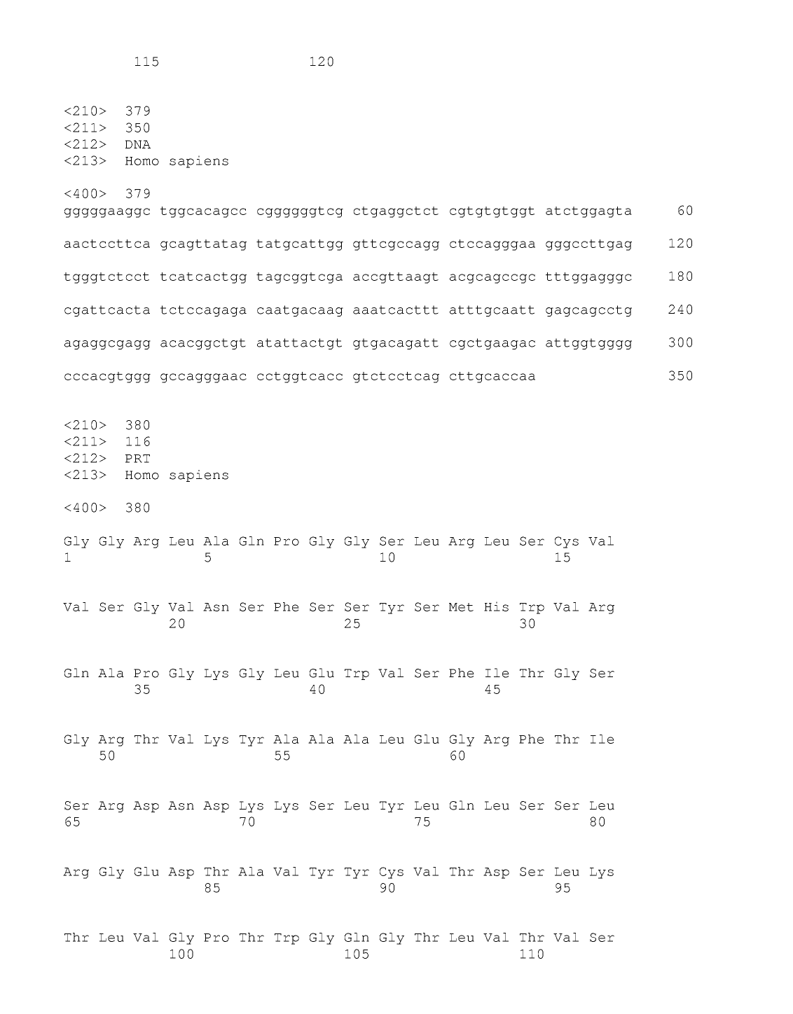

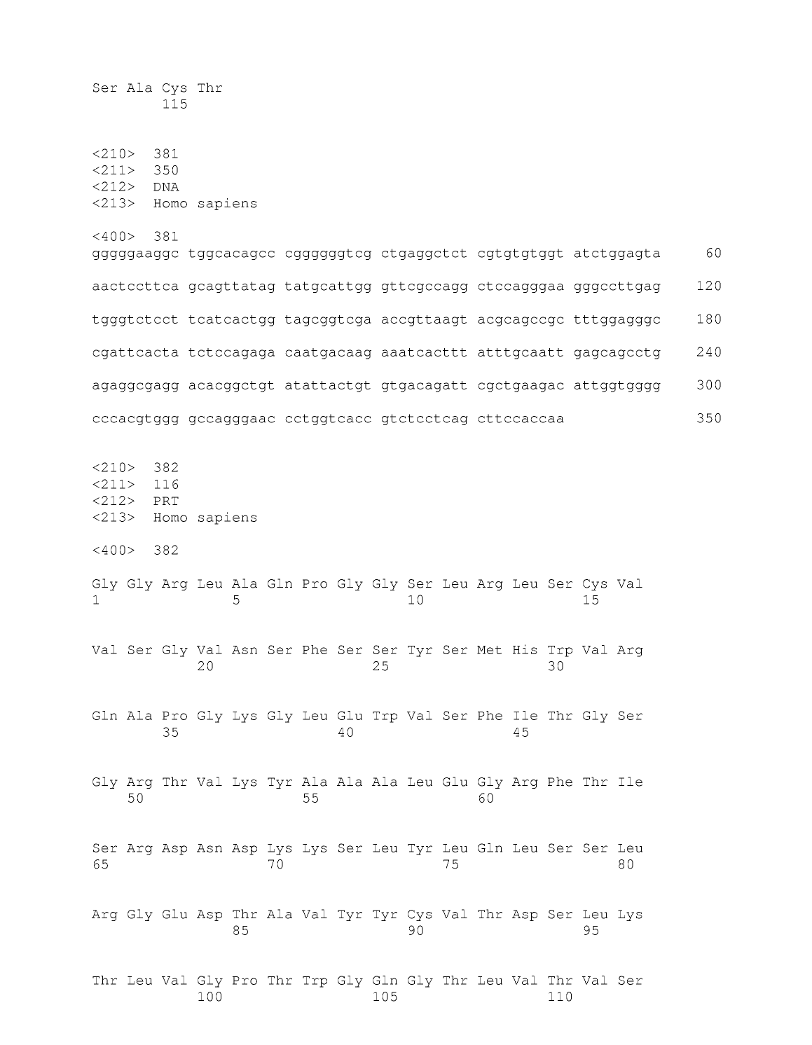

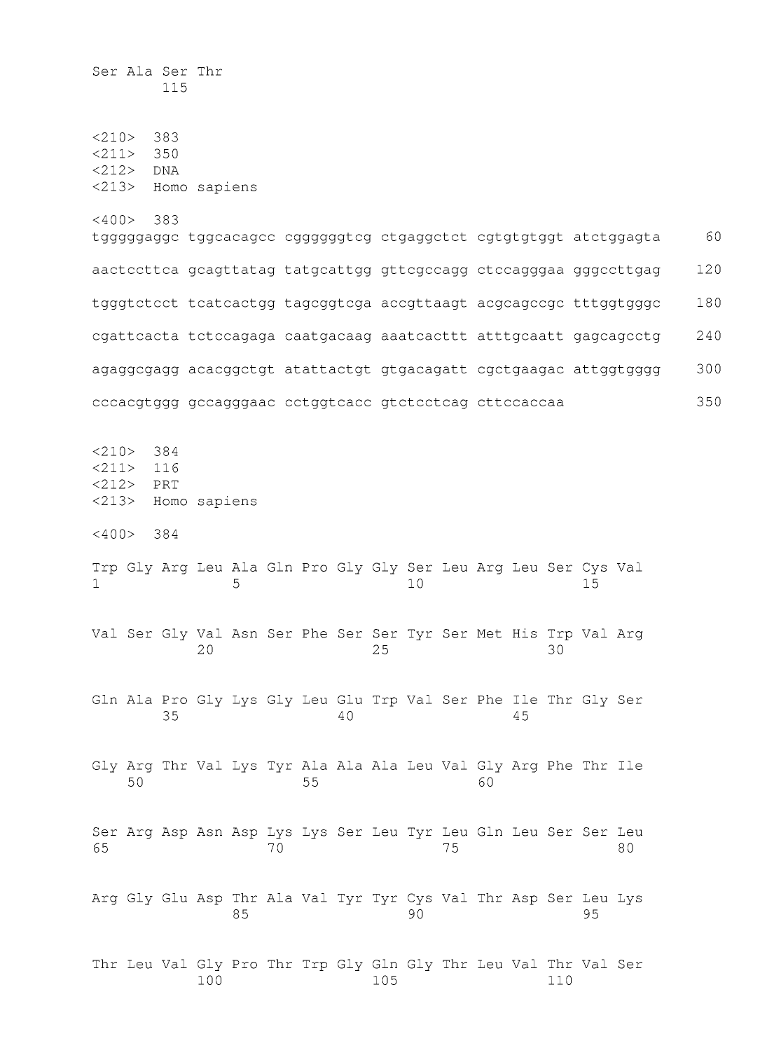

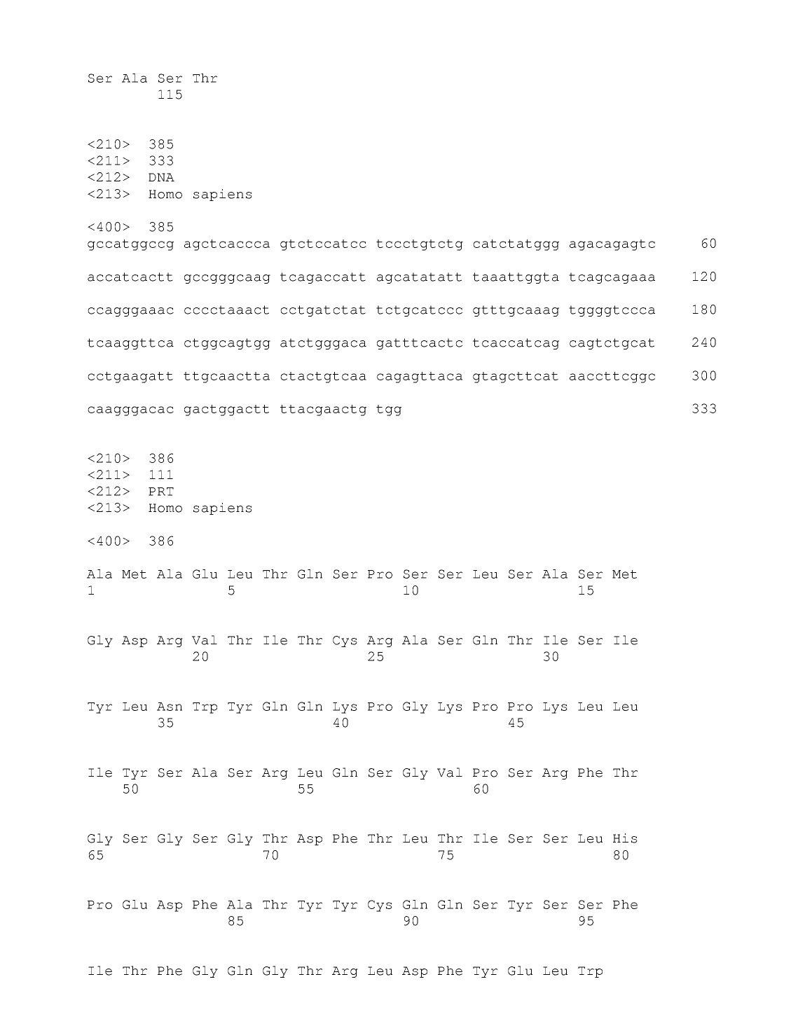

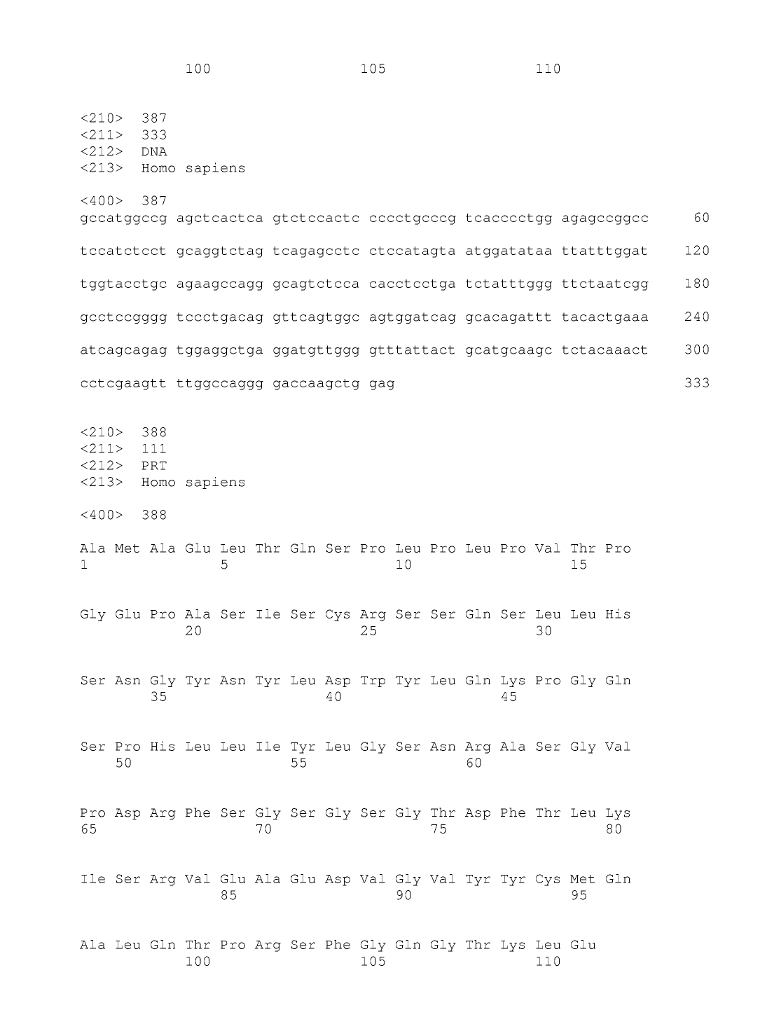

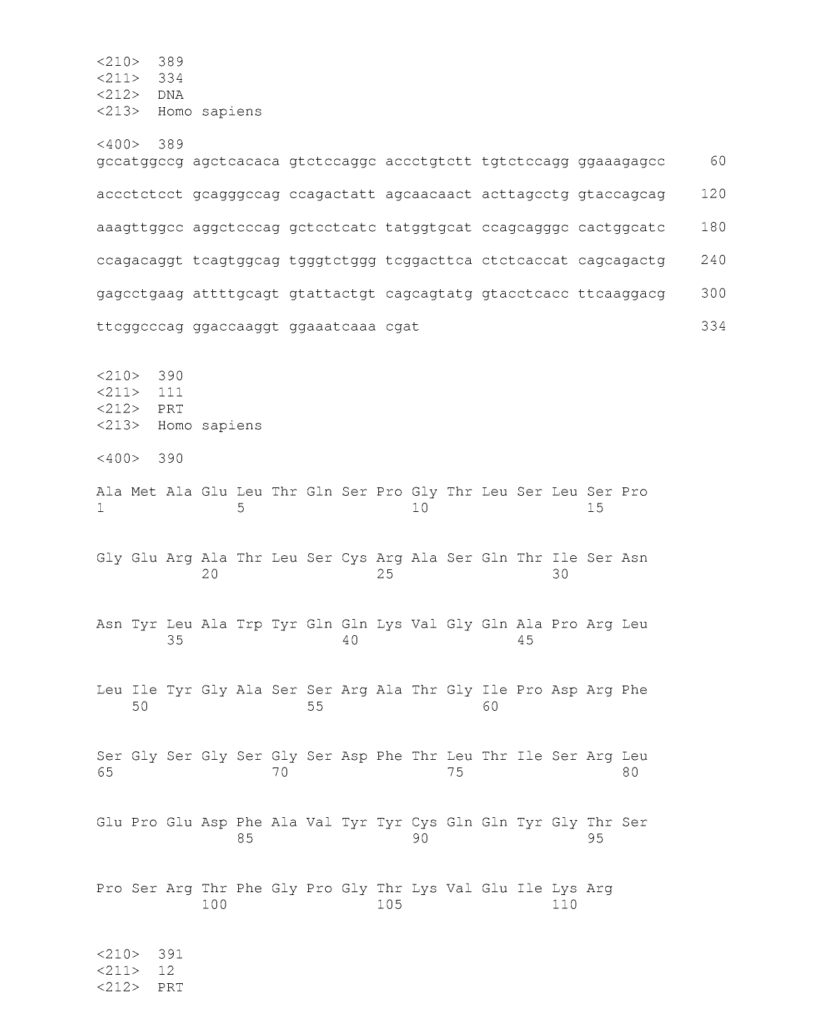

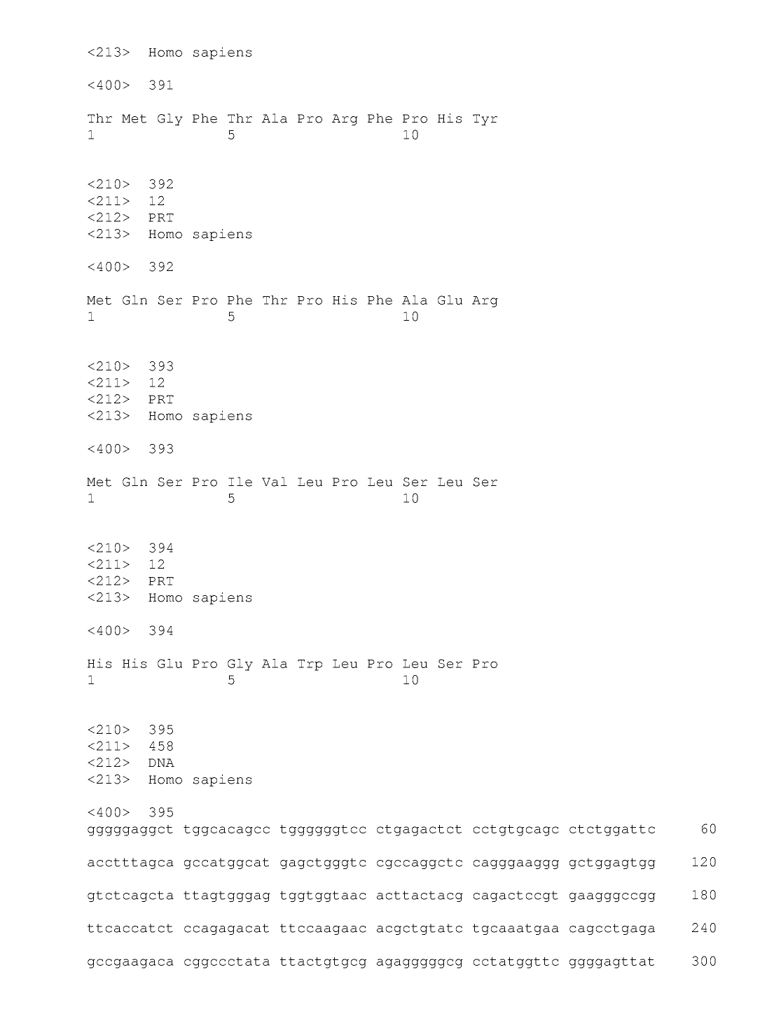

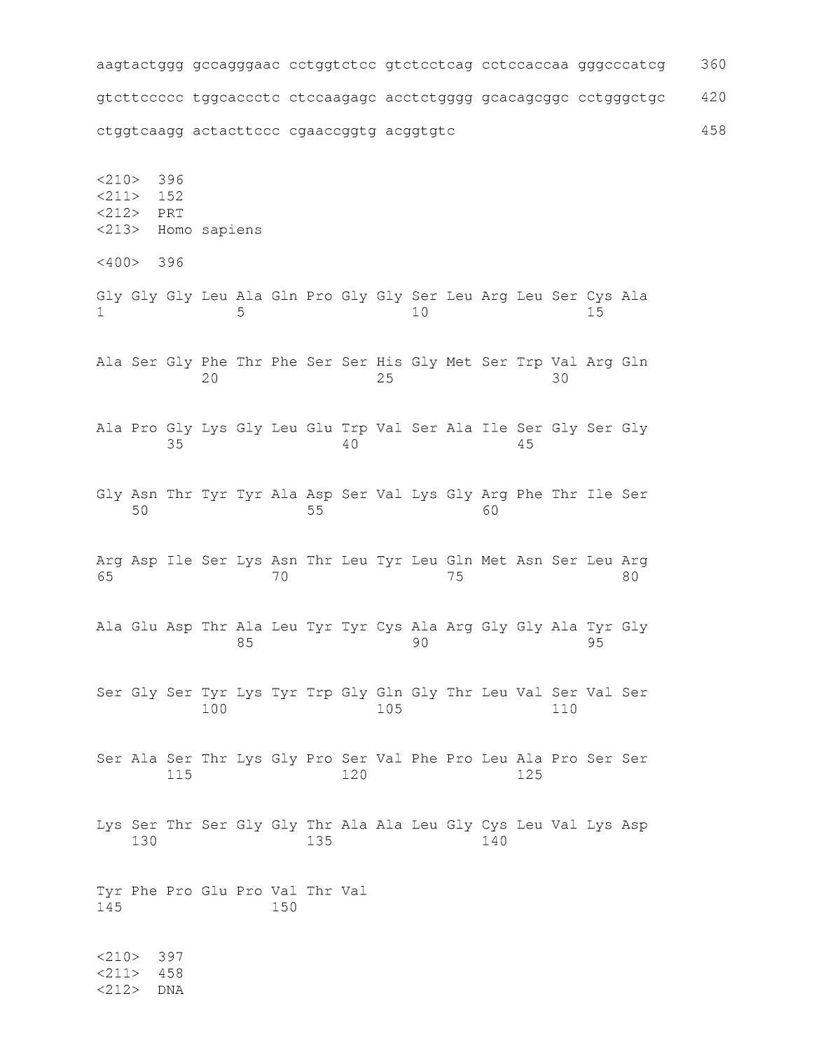

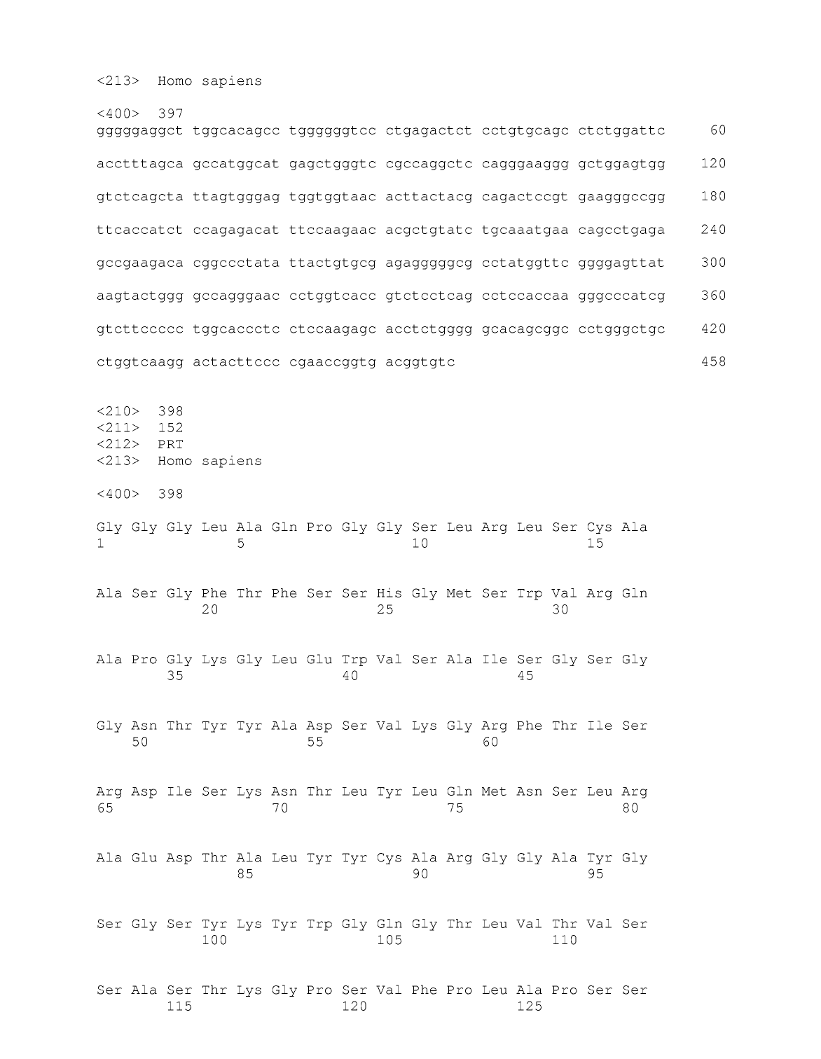

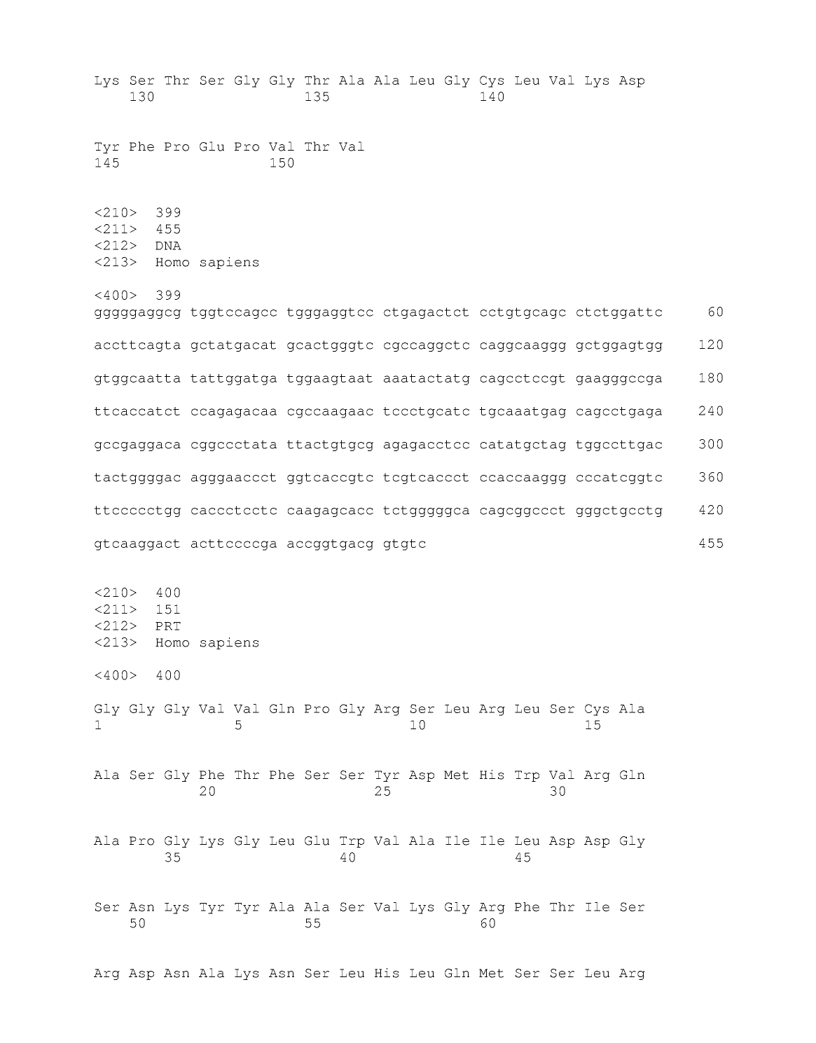

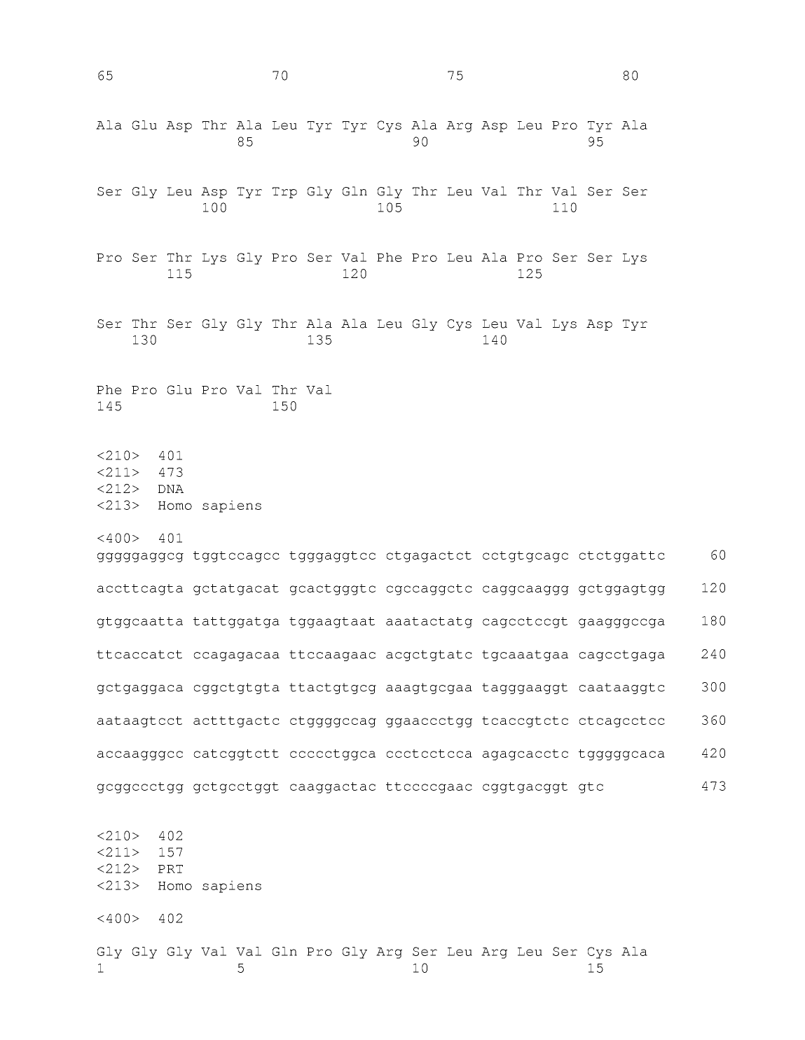

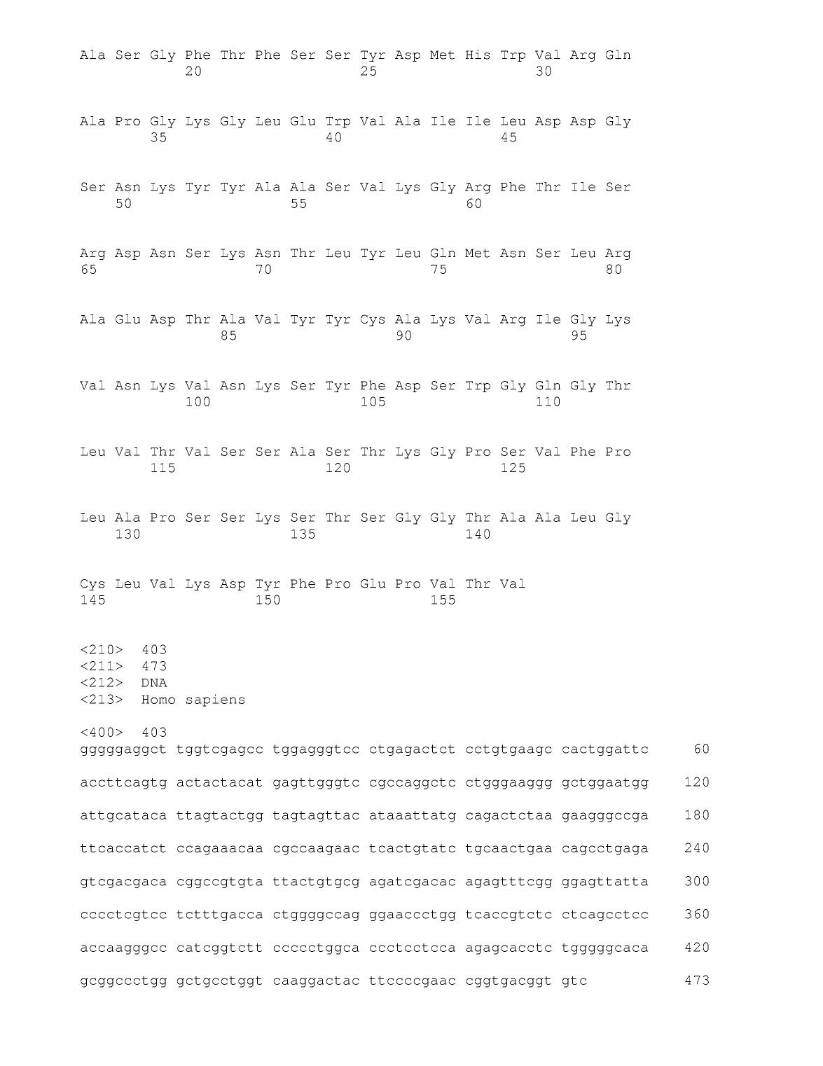

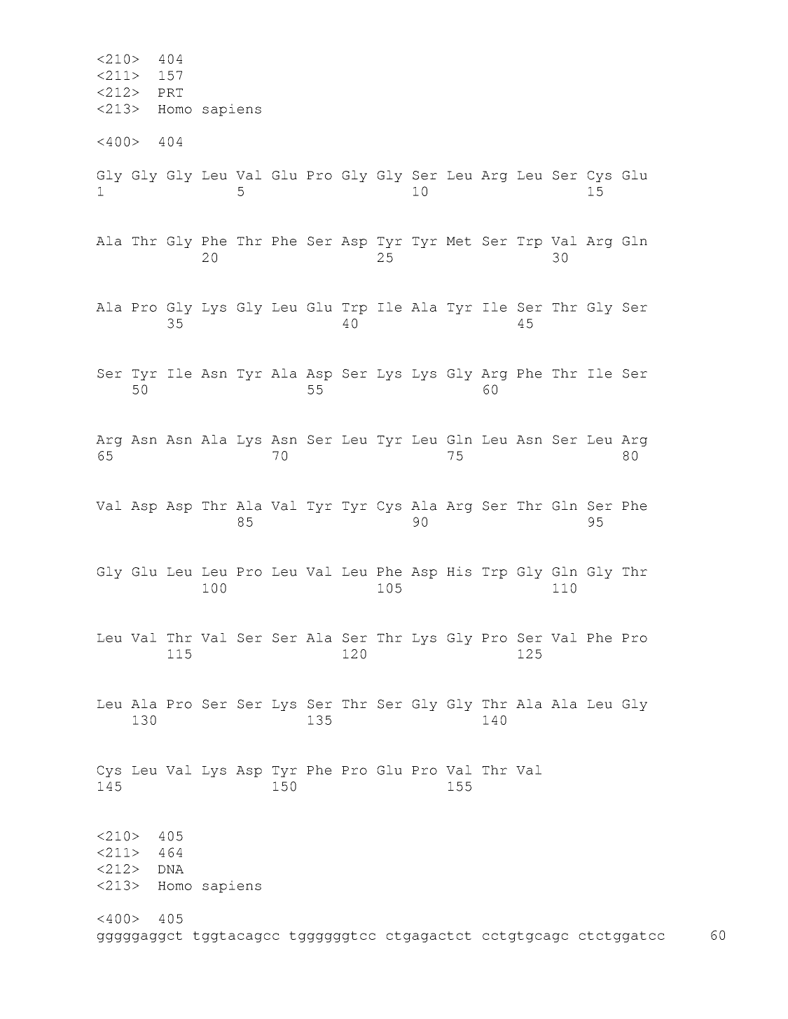

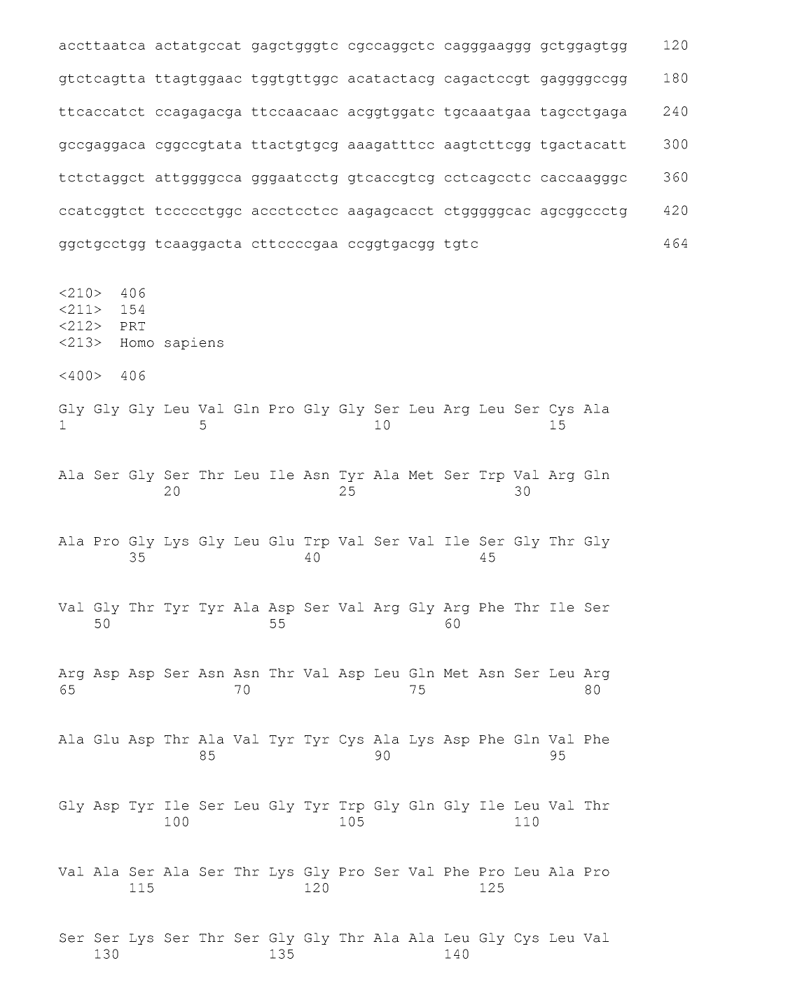

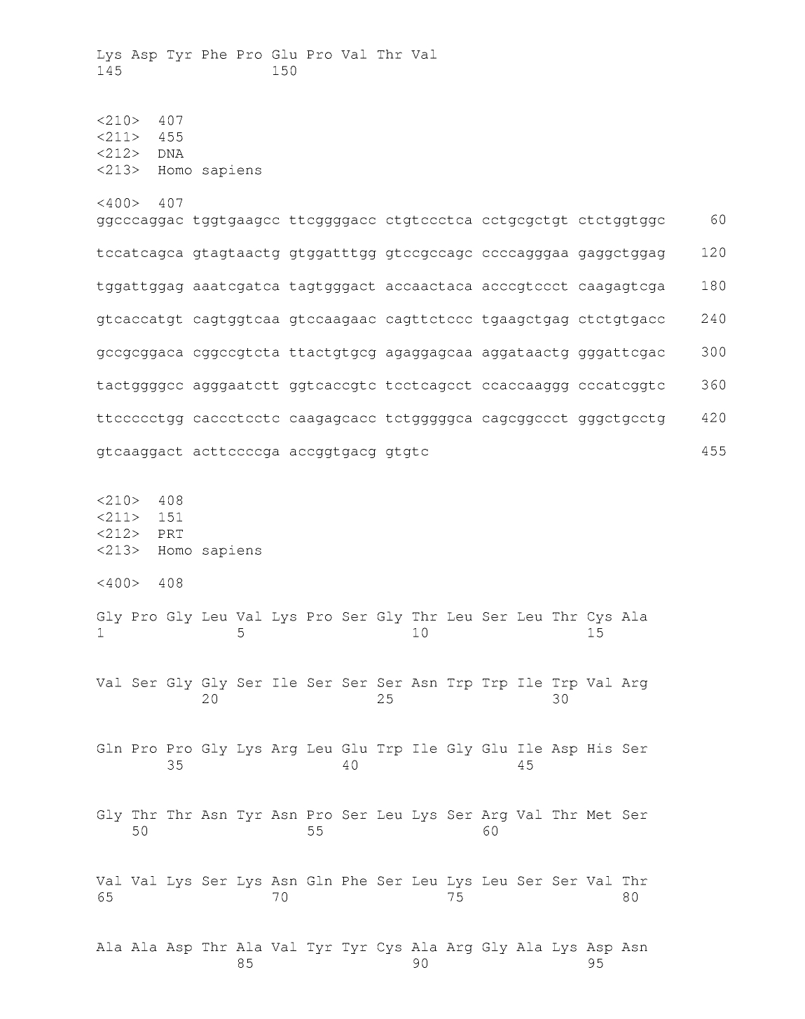

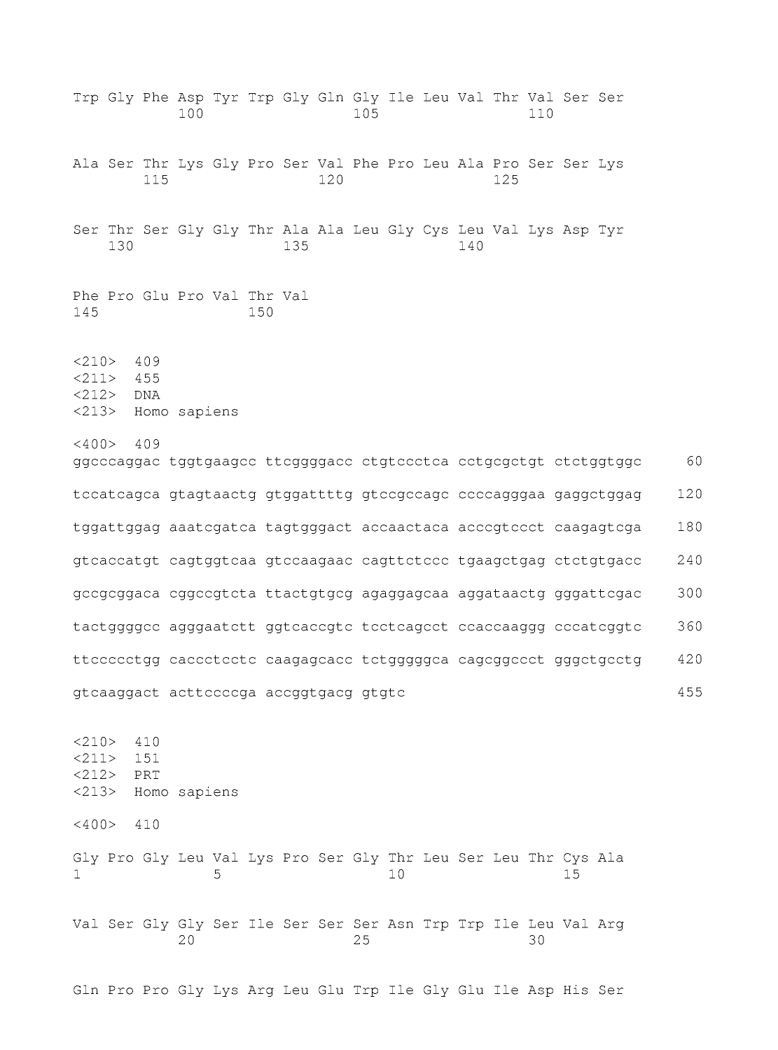

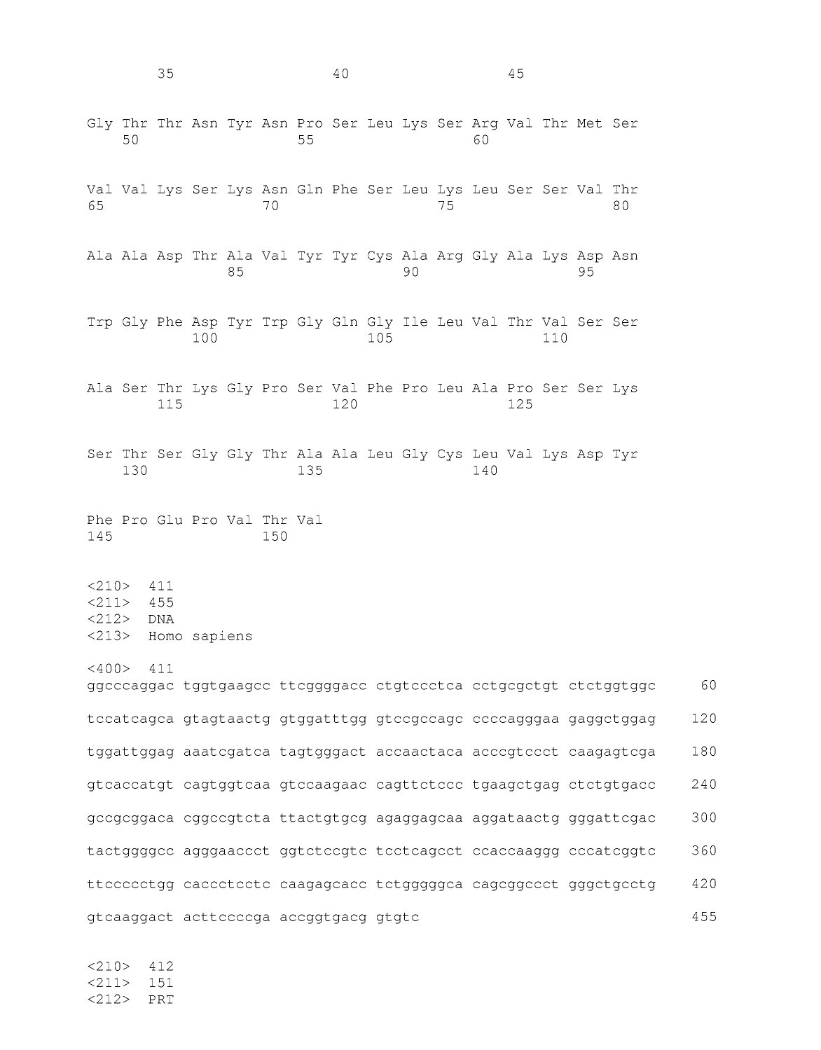

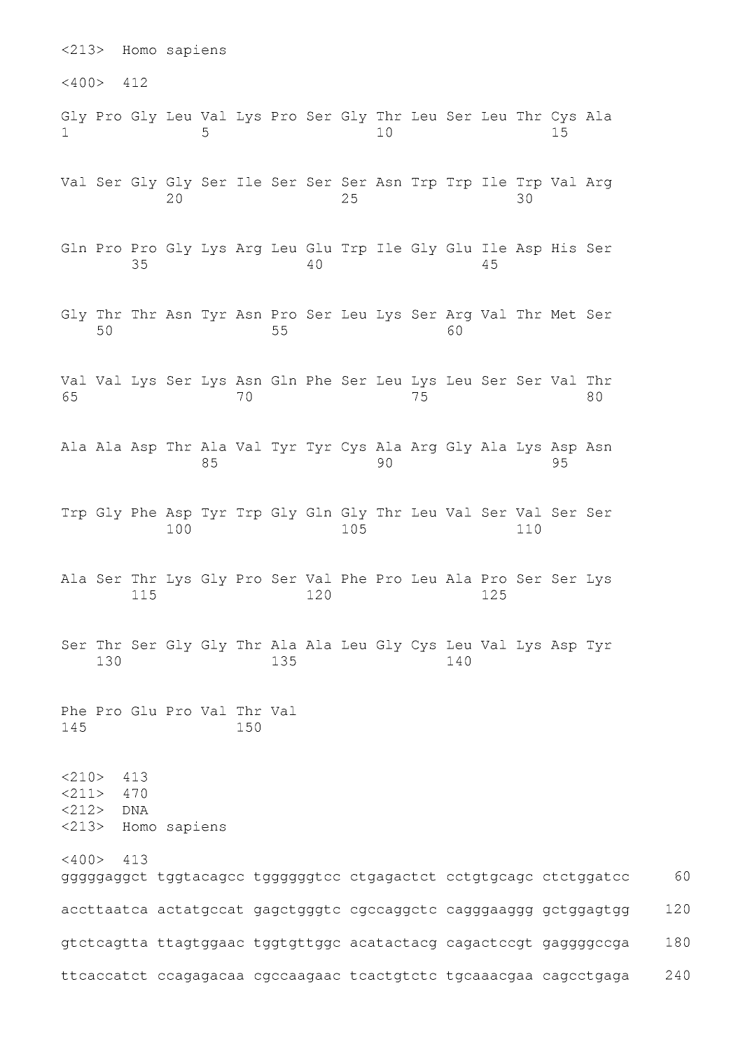

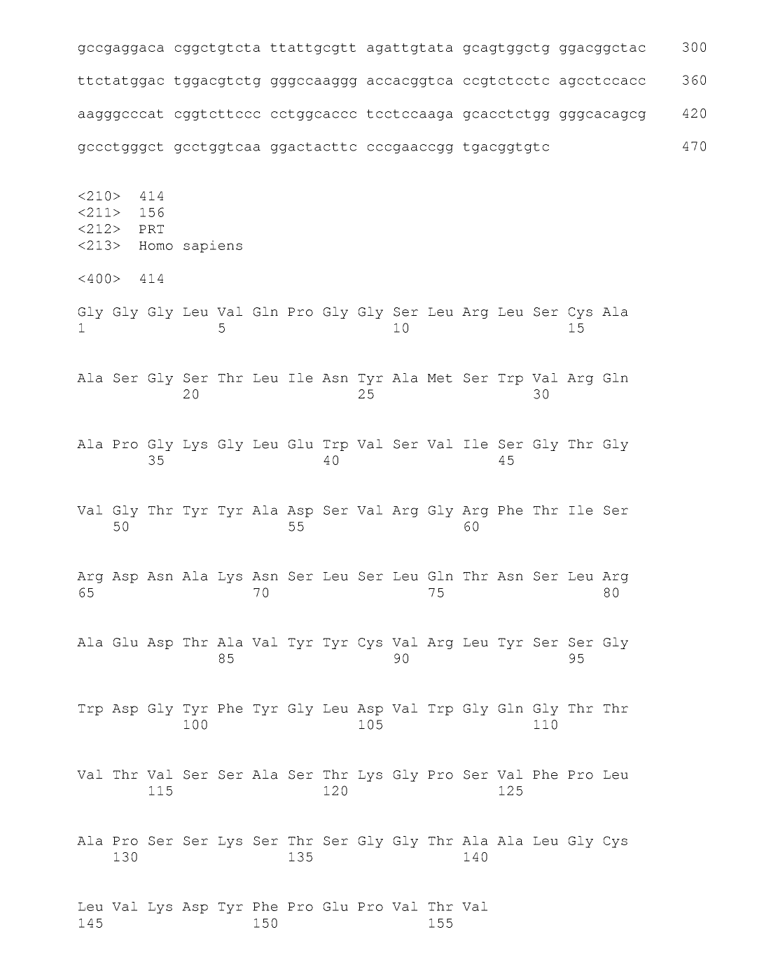

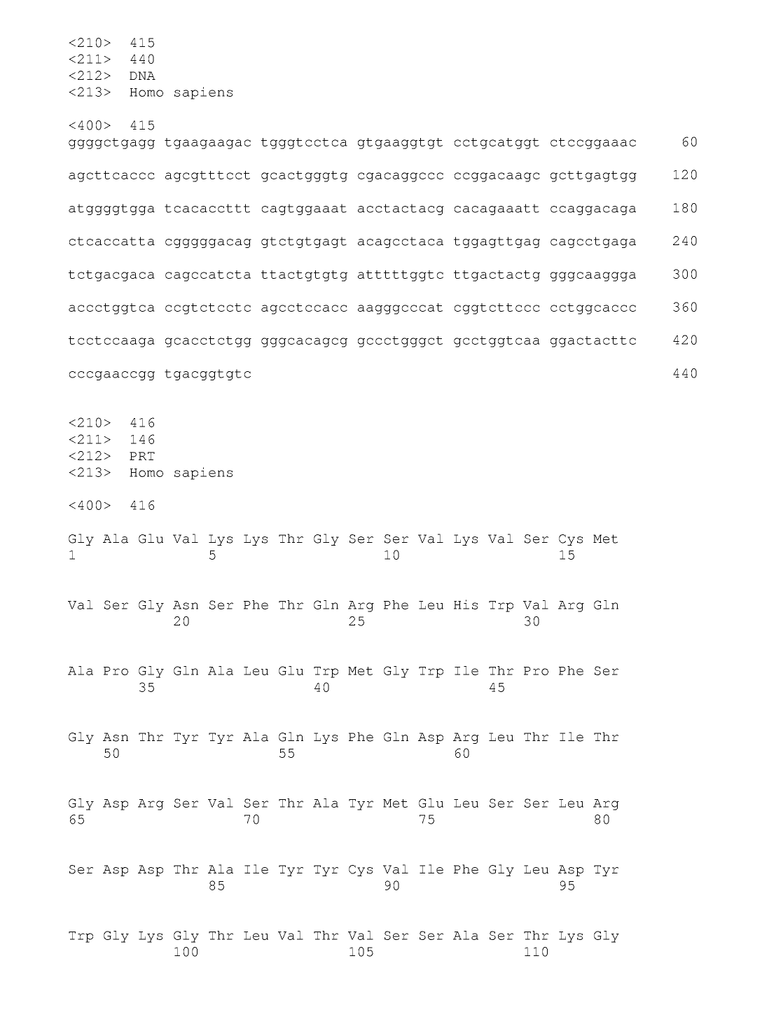

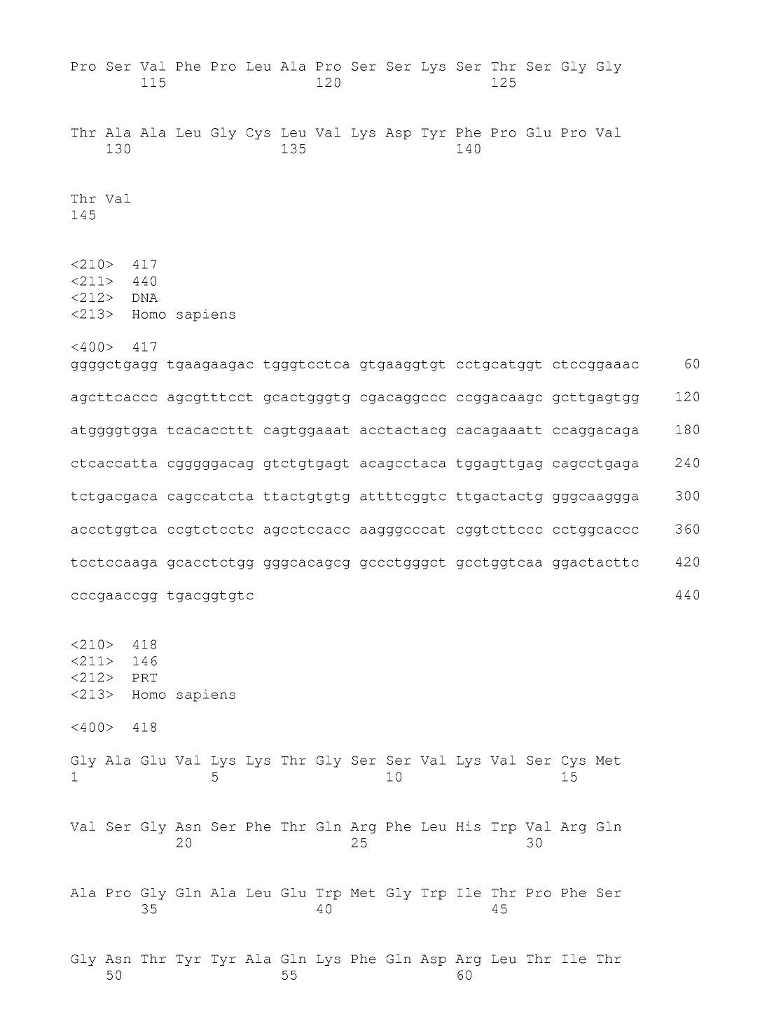

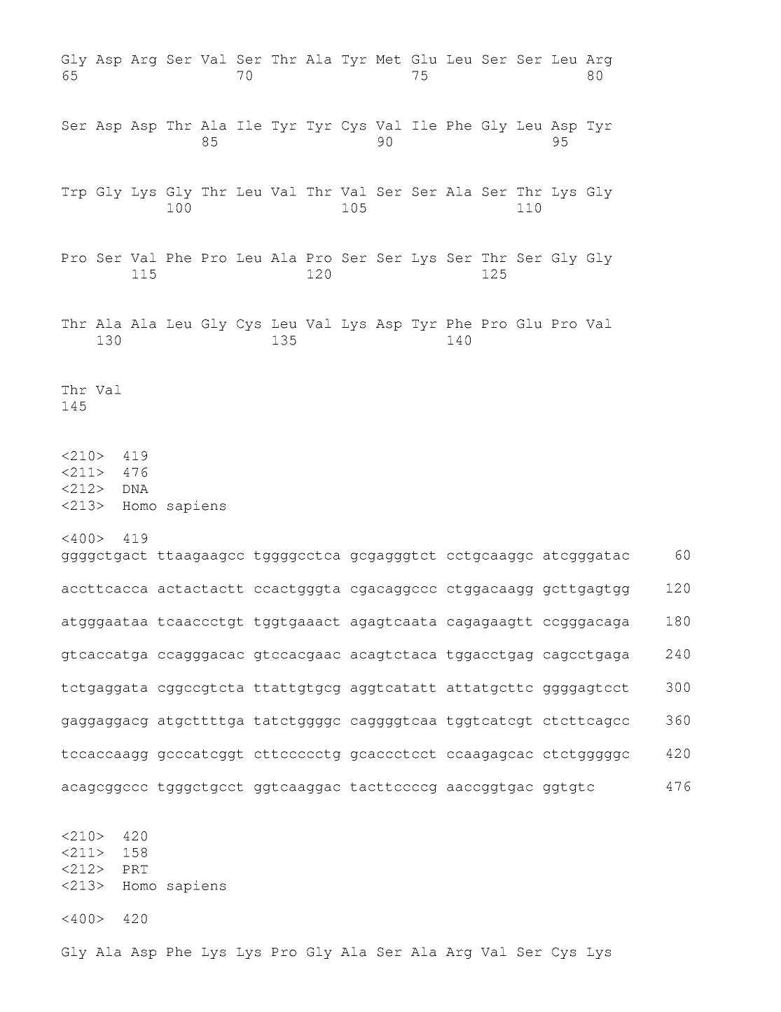

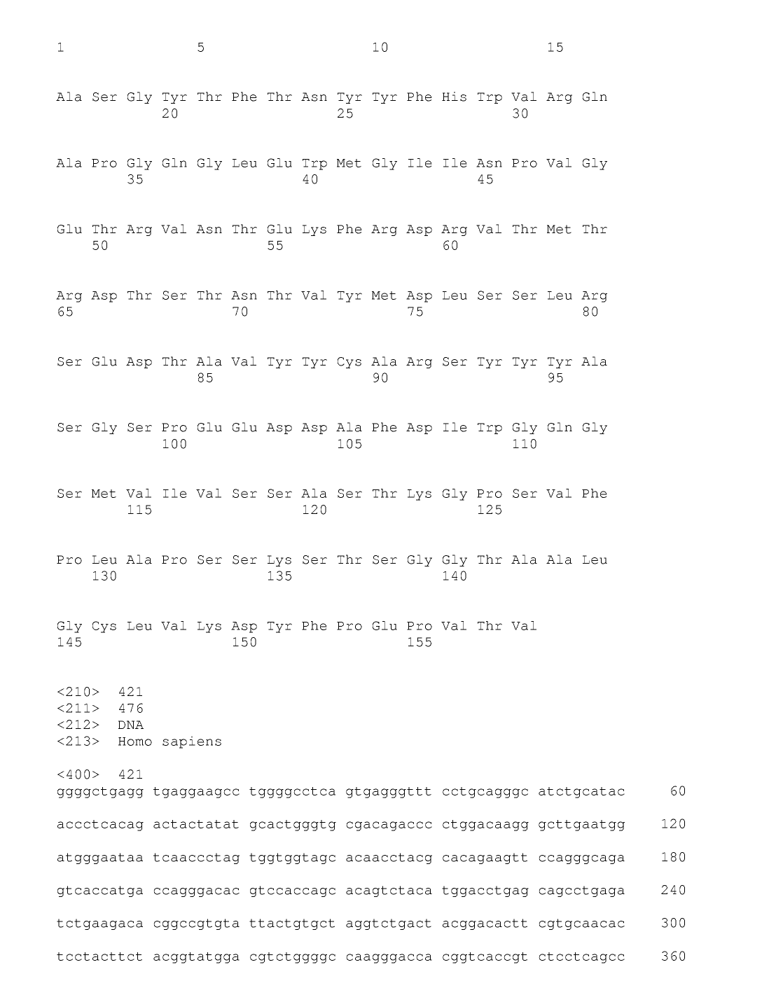

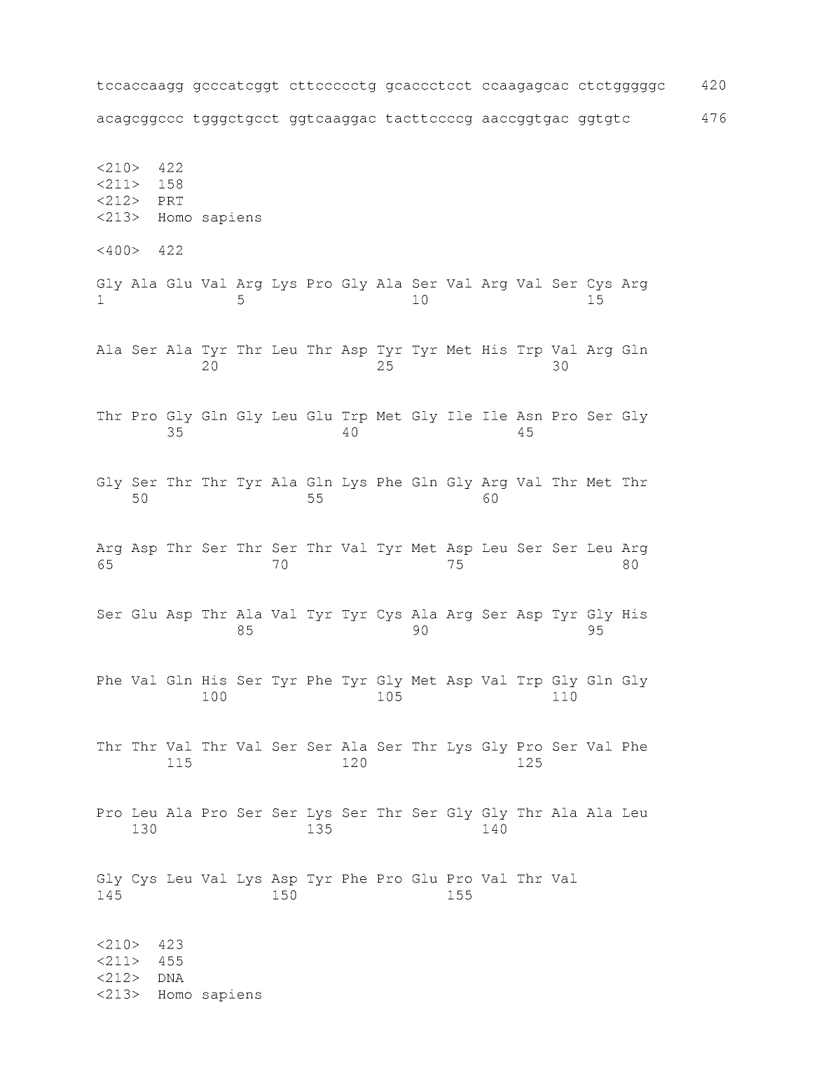

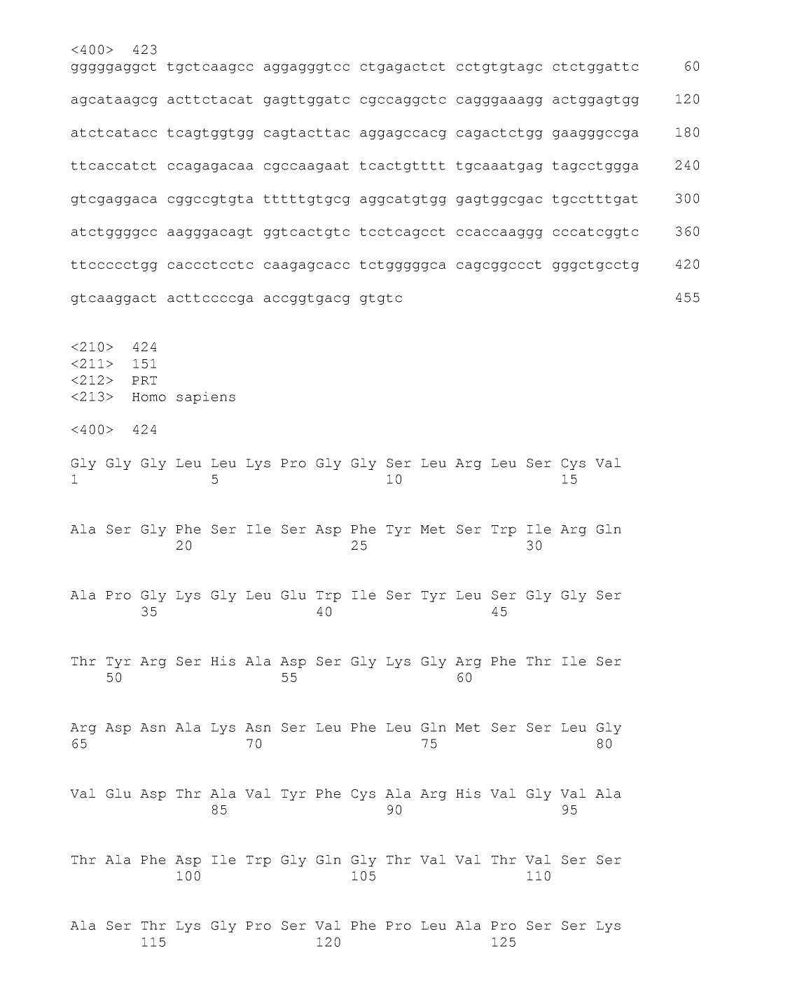

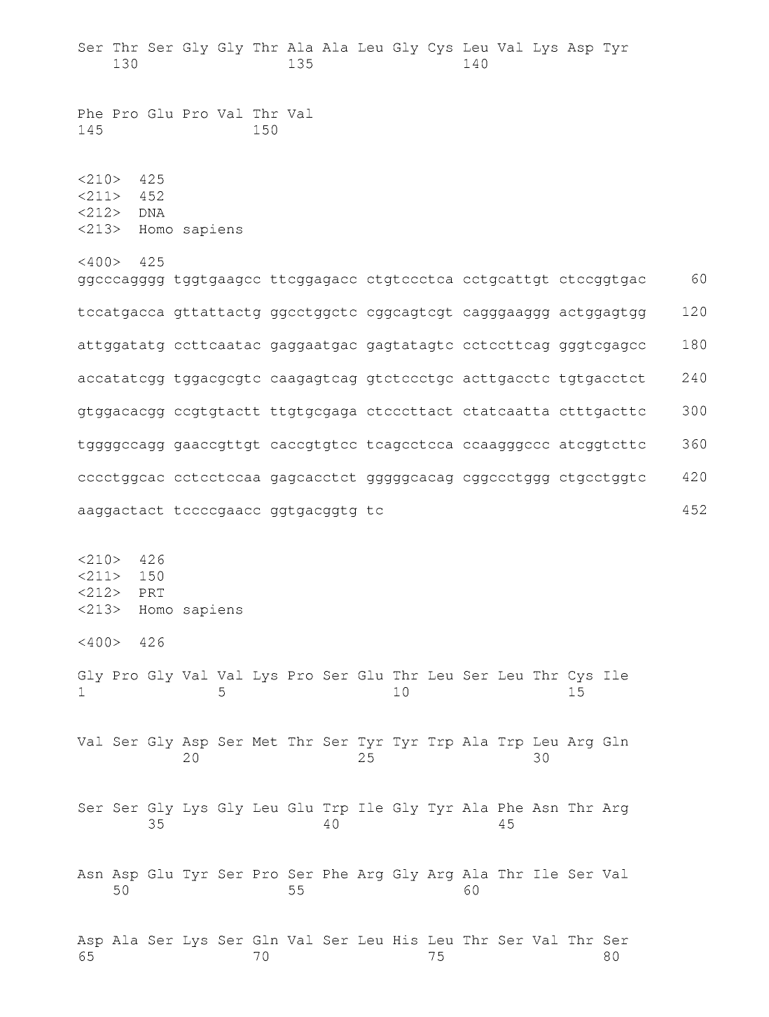

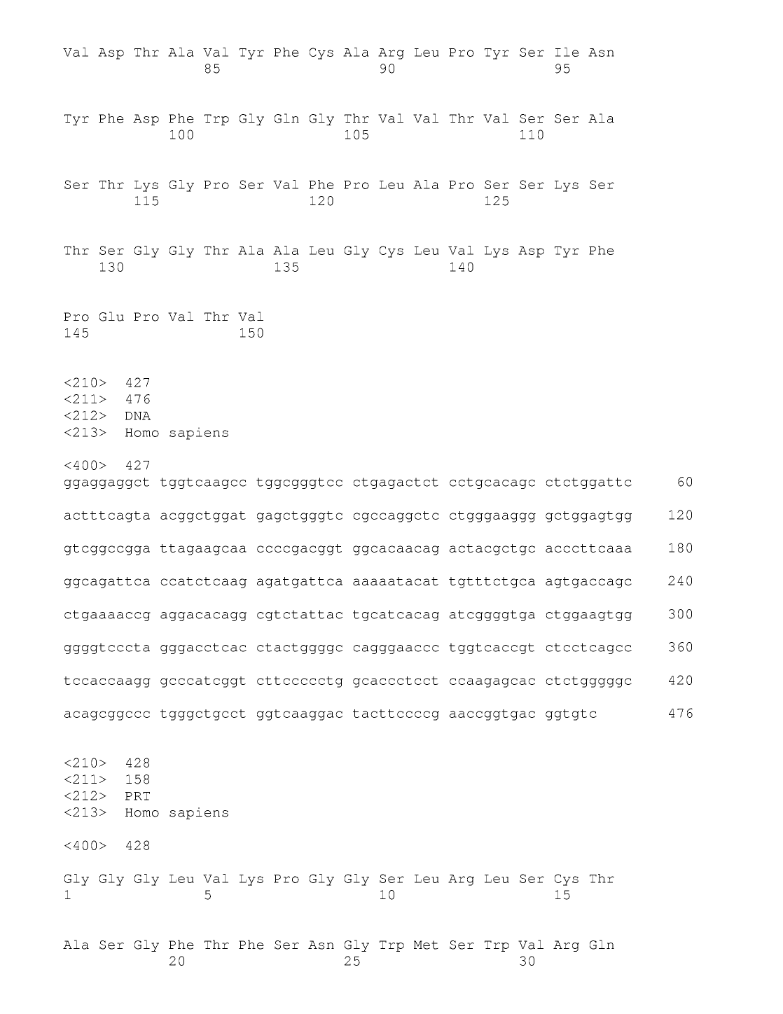

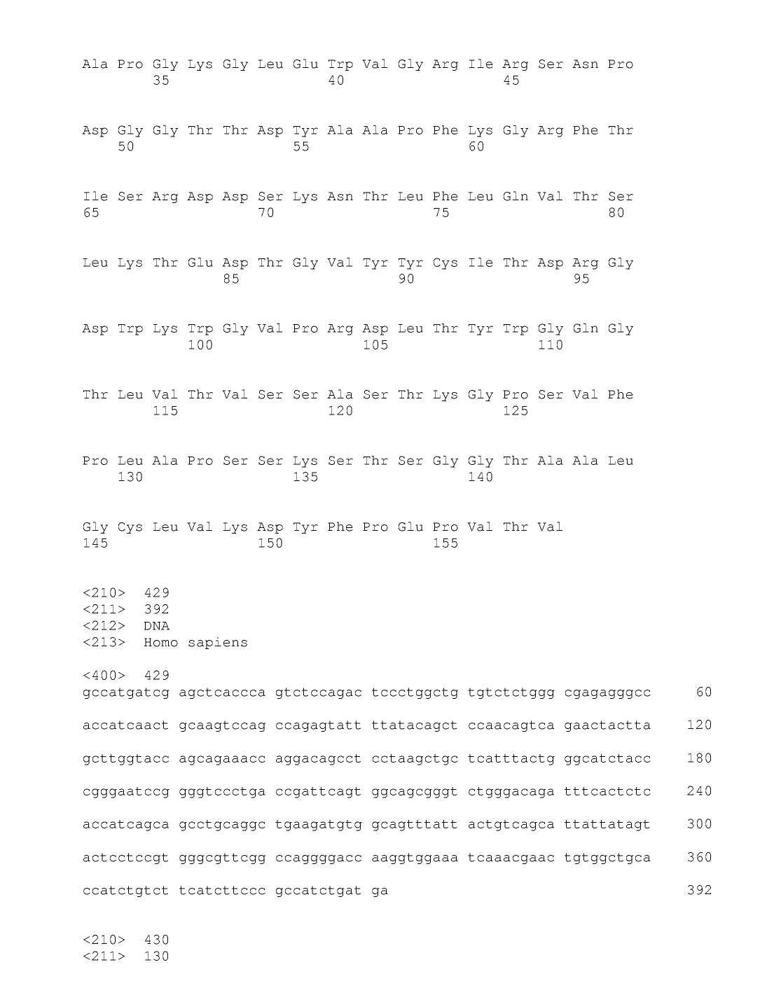

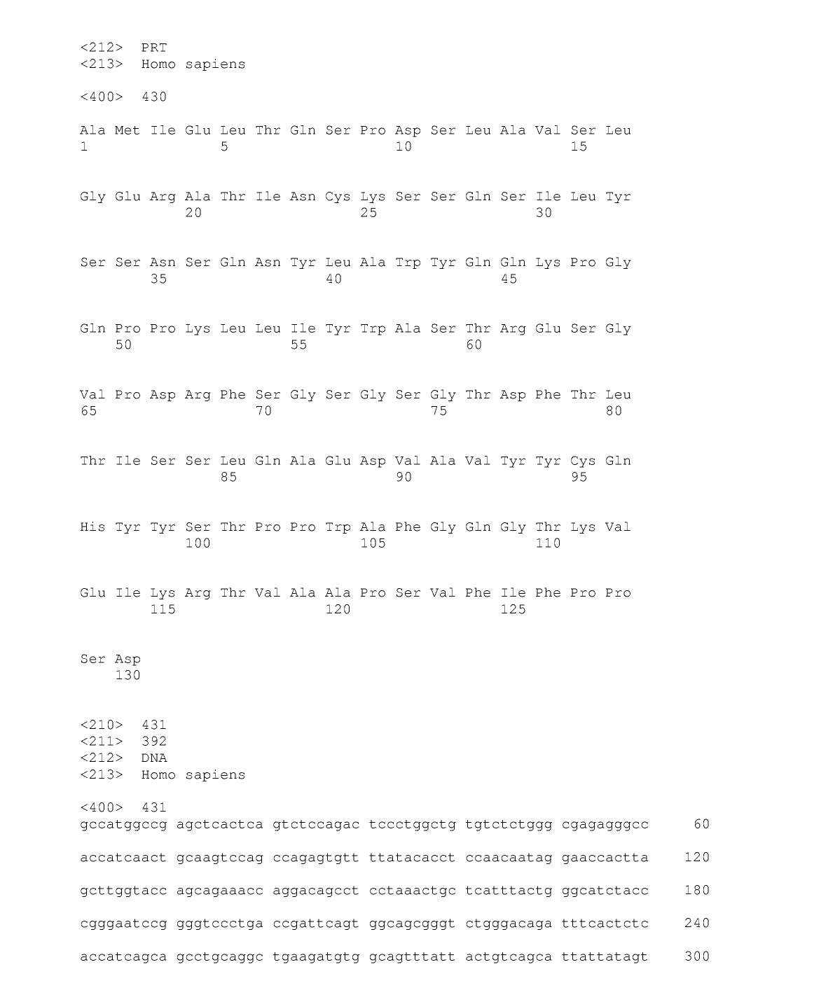

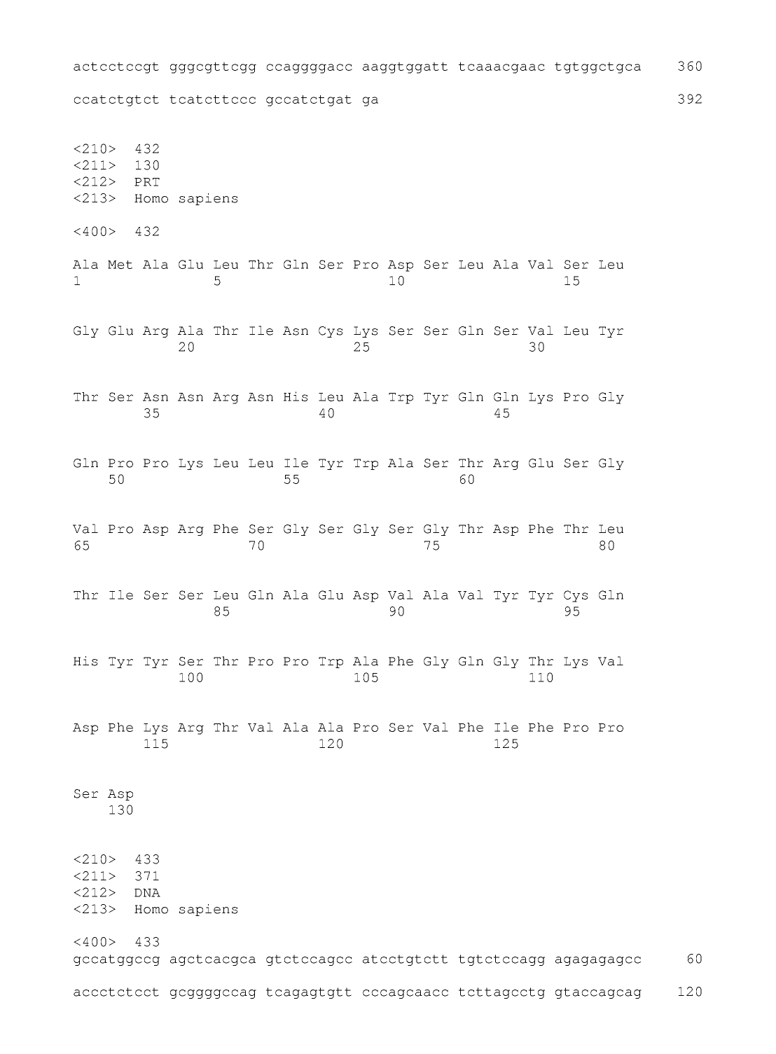

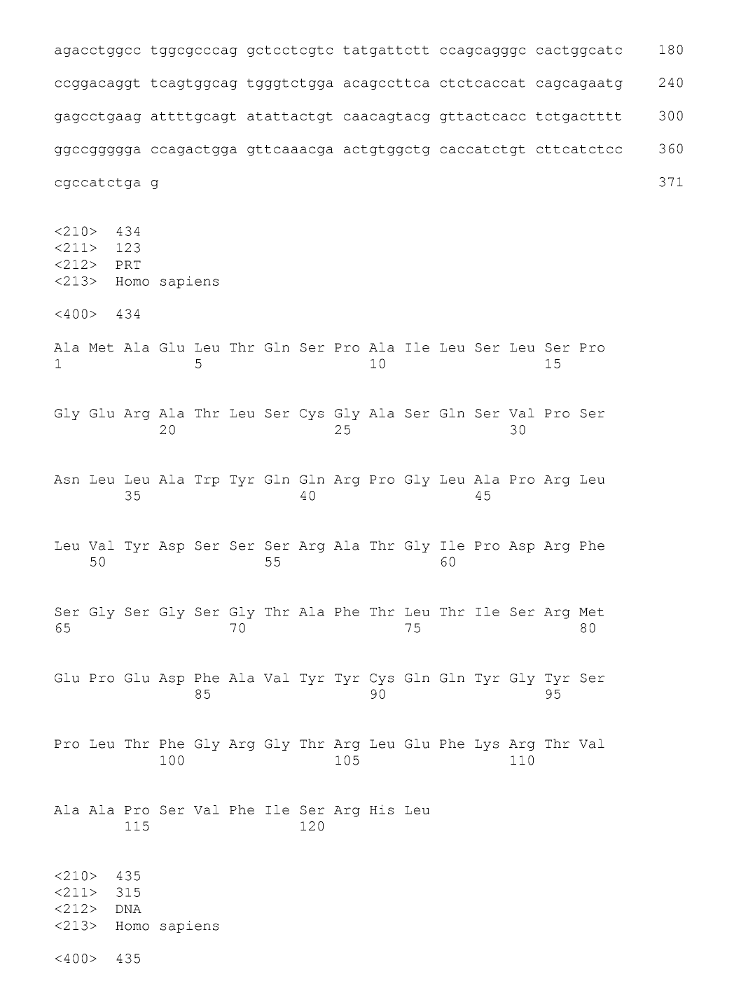

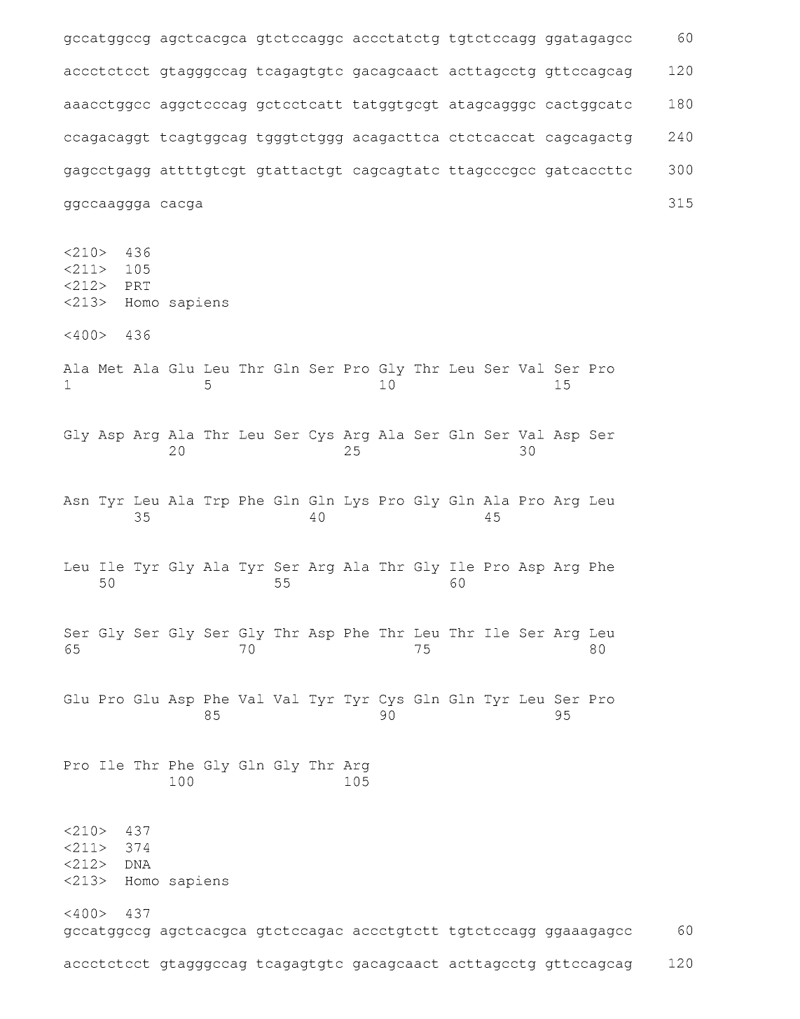

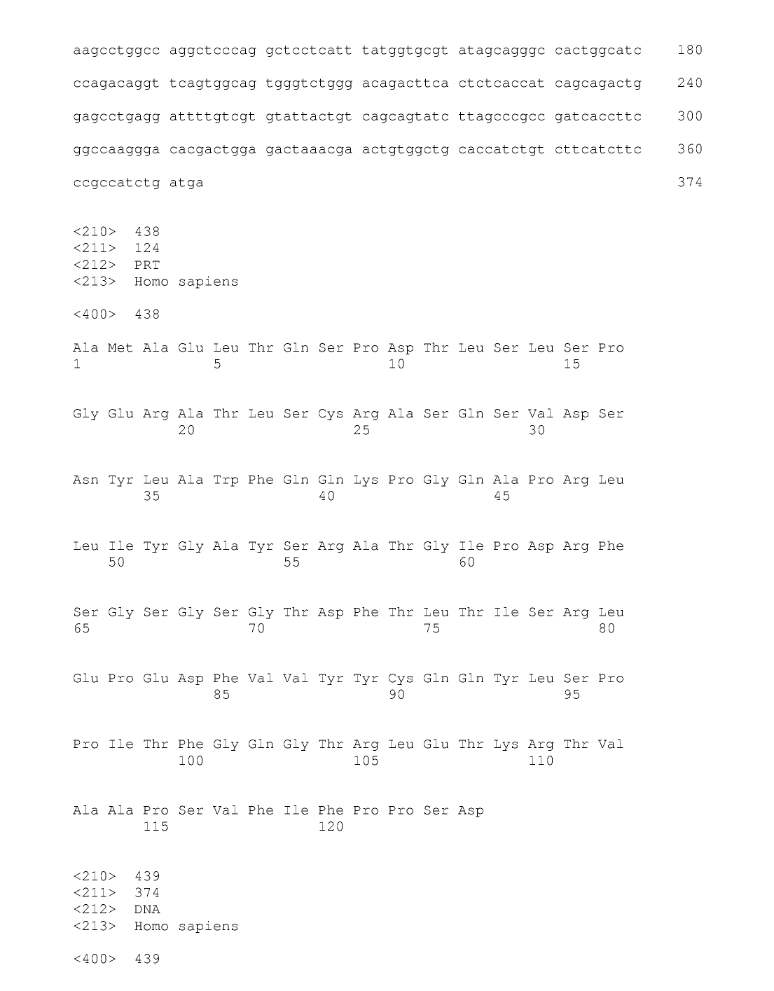

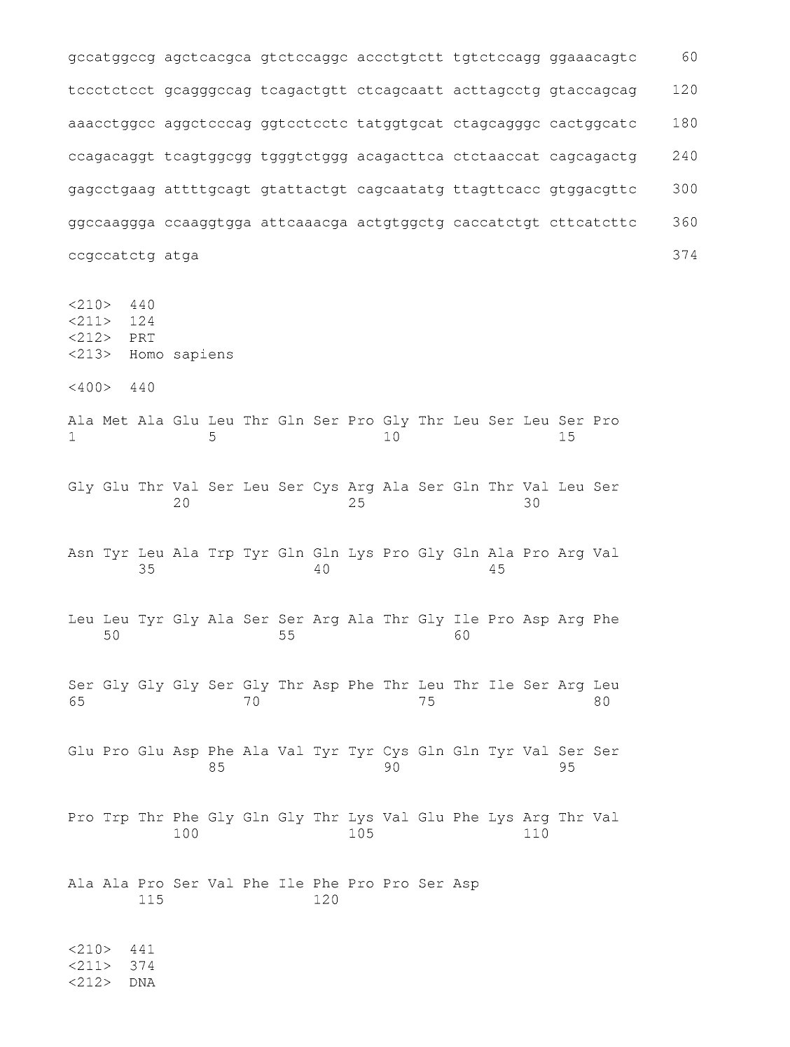

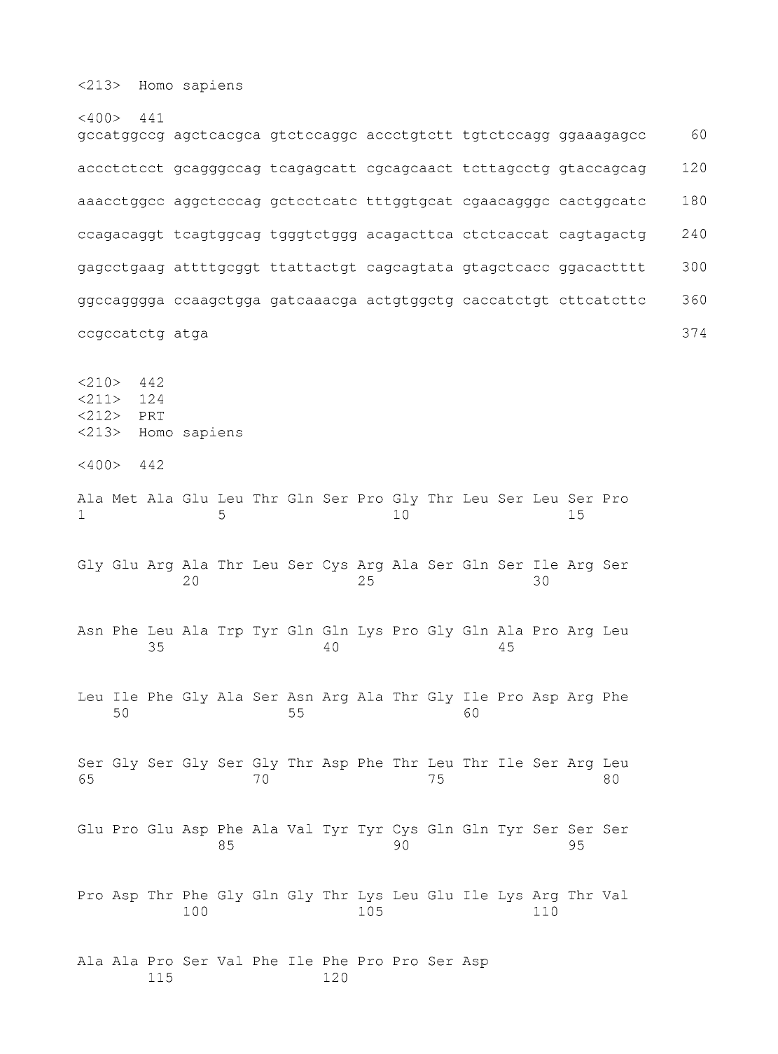

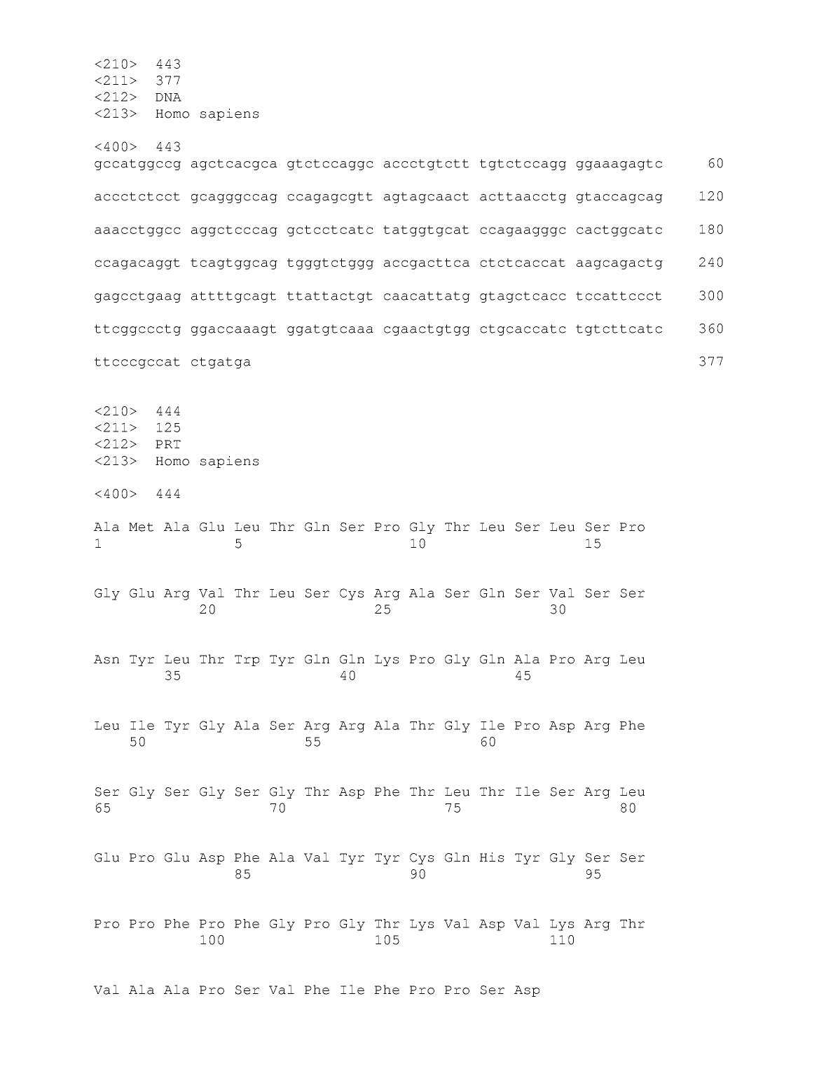

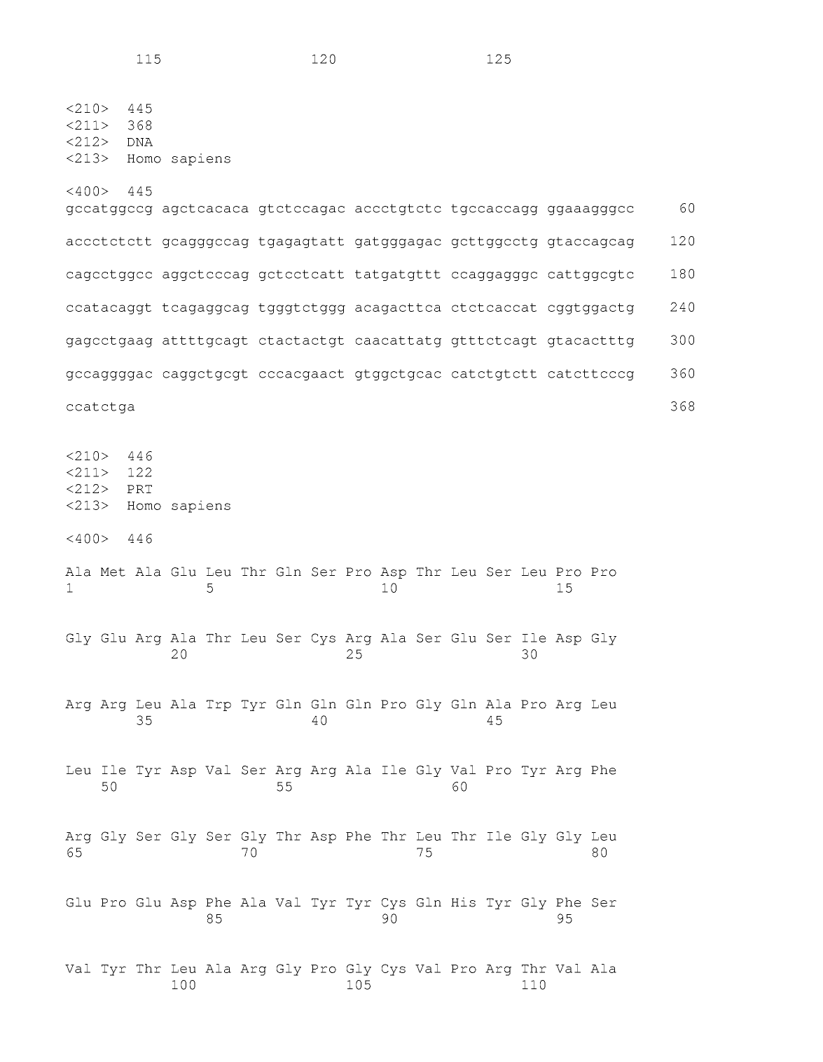

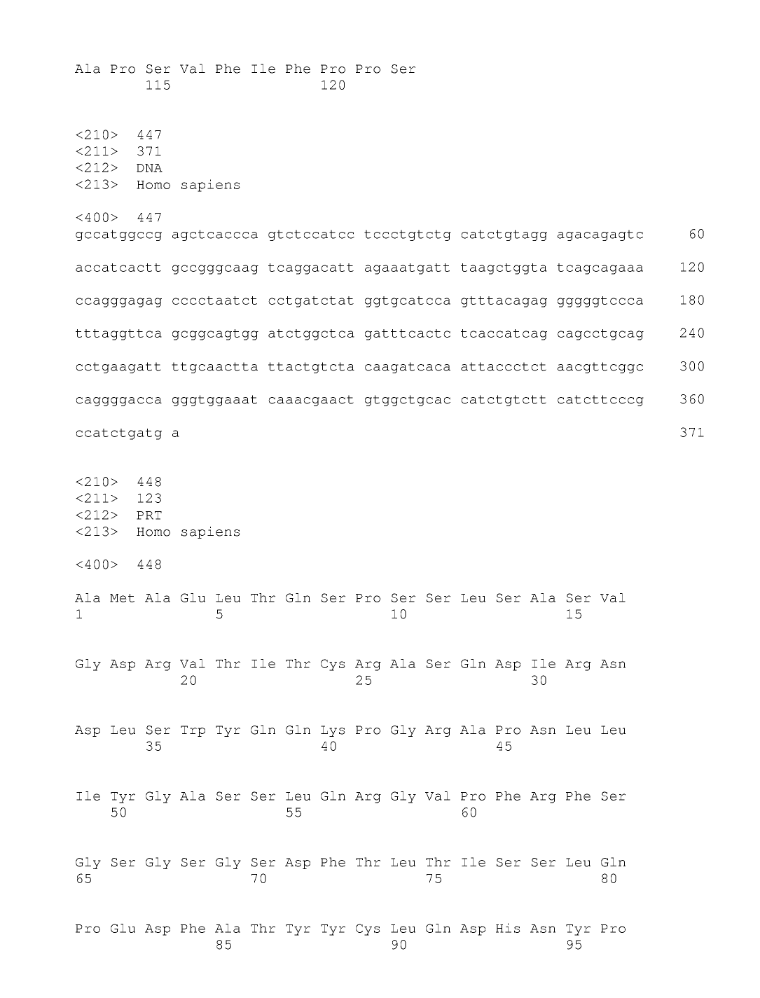

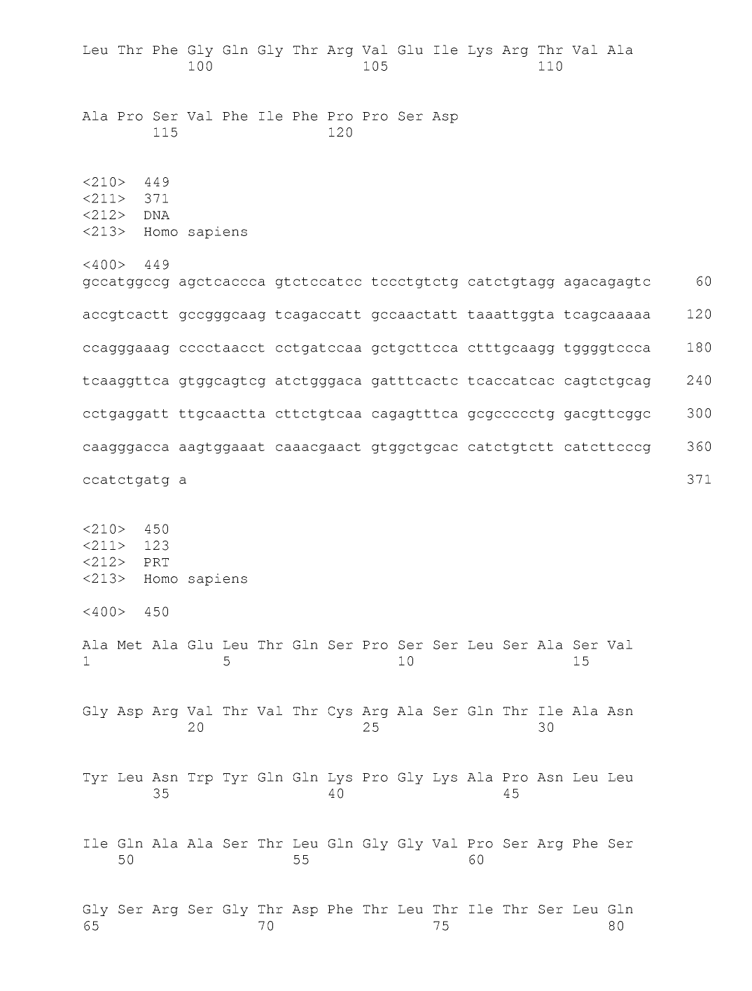

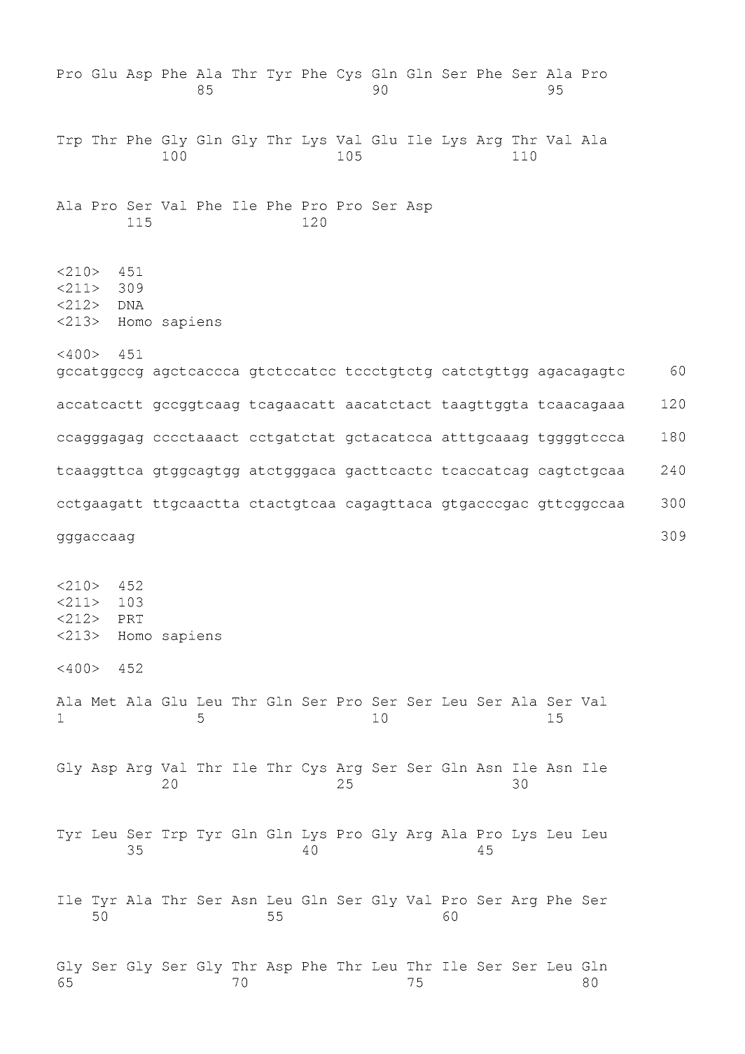

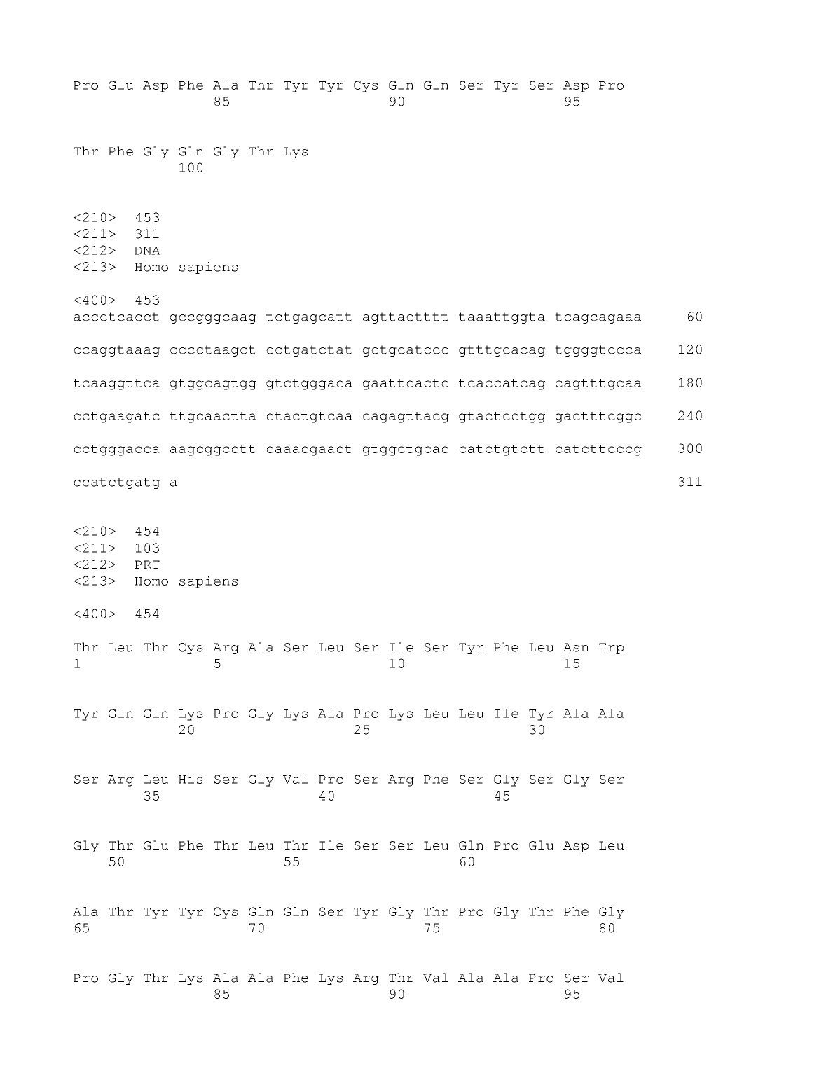

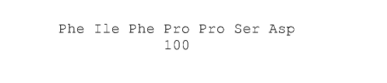

Preferred HC and LC combinations: SEQ ID NOs from Parent Application and Current Application

| Heavy chain | | Light chain | |
|---|---|---|---|
| Parent | Current | Current | Parent |
| application SEQ | Application | Application | application SEQ |
| ID No (FIG. 25) | SEQ ID No | SEQ ID No | ID No (FIG. 25) |
| SEQIDNO: 286 | SEQIDNO: 2 | SEQIDNO: 4 | SEQIDNO: 338 |
| SEQIDNO: 408 | SEQIDNO: 6 | SEQIDNO: 8 | — |
| SEQIDNO: 428 | SEQIDNO: 10 | SEQIDNO: 12 | SEQIDNO: 438 |
| SEQIDNO: 44 | SEQIDNO: 14 | SEQIDNO: 16 | SEQIDNO: 54 |
| SEQIDNO: 416 | SEQIDNO: 18 | SEQIDNO: 30 | SEQIDNO: 444 |
| SEQIDNO: 416 | SEQIDNO: 18 | SEQIDNO: 32 | SEQIDNO: 442 |
| — | SEQIDNO: 20 | SEQIDNO: 32 | SEQIDNO: 442 |
| — | SEQIDNO: 20 | SEQIDNO: 34 | SEQIDNO: 450 |
| — | SEQIDNO: 22 | SEQIDNO: 32 | SEQIDNO: 442 |
| — | SEQIDNO: 24 | SEQIDNO: 30 | SEQIDNO: 444 |
| SEQIDNO: 398 | SEQIDNO: 26 | SEQIDNO: 30 | SEQIDNO: 444 |
| SEQIDNO: 402 | SEQIDNO: 28 | SEQIDNO: 12 | SEQIDNO: 438 |
| SEQIDNO: 402 | SEQIDNO: 28 | SEQIDNO: 30 | SEQIDNO: 444 |
| SEQIDNO: 402 | SEQIDNO: 28 | SEQIDNO: 32 | SEQIDNO: 442 |

### DETAILED DESCRIPTION OF THE INVENTION

The present invention refers to human antibodies characterized by the ability to bind transgelin (called also SM22) as well as an outer membrane bacterial protein with a high degree of homology to OmpK36 (Outer membrane protein Klebsiella 36, GI:295881594). Preferably said bacterial membrane protein shows at least 50% similarity to OmpK36 (Outer membrane protein, Klebsiella, K36; GI: 295881594) or fragments thereof.

Transgelin means proteins having at least 80% similarity to trangelin-1 (Accession No Q01995 (Uniprot), GI:48255907 (NCBI). Thus, the term transgelin encompasses transgelin-2 (Accession No P37802 (Uniprot), GI:12803567 (NCBI) and transgelin-3 (Accession No Q9U115 (Uniprot), GI:15929818 (NCBI)). Transgelin, in particular transgelin 1, has been called in the past also SM22 and is encoded by the gene TAGLN. Particularly preferred among transgelins is transgelin 1 as identified above.

The antibodies herein disclosed are endowed with the further property of binding an antigen in the atherosclerotic plaque and are thus useful diagnostic reagents for atherogenic disorders. By atherogenic disorders are meant disorders leading to an atheromatous disease such as those selected from the group consisting of:
atherogenic ischemic or occlusive evolution in an arterial vessel;
Acute Coronary Syndrome comprising: unstable angina, ST Elevation Myocardial Infarction (STEMI), non STEMI myocardial infarction and related cardiovascular diseases,
intra-cerebral occlusive diseases;
peripheral artery occlusive diseases; and
non acute coronary diseases.

According to a preferred embodiment, the antibodies are human and recombinant antibodies selected from human antibody heavy or light chain libraries prepared from atherosclerotic plaque samples, as described in WO2009/037297 and U.S. Ser. No. 12/679,109, incorporated herein by reference in their entirety. They possess the additional advantages deriving from being from a human source, important for example, in therapeutic applications. Moreover, due to their being developed within the atherosclerotic plaque by an antigen affinity driven selection they provide a hint, if not a molecular image, of the antigens involved in the in vivo mechanisms therein activated, representing thus unique research tools.

The terms antibody and binding agents according to the present invention are used with equivalent meaning: they refer to reagents for which a specific recognition pattern for both transgelin and OmpK36 can be detected according to any of the immunologic techniques described in the following.

According to a preferred embodiment, antibodies binding to an antigen in the atherosclerotic plaque is detected by either binding to a purified, preferably recombinant, protein or fragments thereof or to an atherosclerotic plaque lysate or an immunohistology section.

According to a preferred embodiment, antibodies of the present invention are a combination of a Heavy variable chain comprising a sequence selected from the group consisting of: SEQ ID NO: 2, 6 and 10 (SEQ ID NOs:286, 408 and 428) and of a light chain variable chain comprising a sequence selected from the group consisting of: SEQ ID NO: 4 (SEQ ID NO:338), 8 and 12 (SEQ ID NO:438). Even more preferably they comprise the following heavy and light chain variable region combinations: SEQ ID NO: 2 and SEQ ID NO:4 (SEQ ID NO:286 and 338), SEQ ID NO: 6 (SEQ ID NO:408) and SEQ ID NO:8; SEQ ID NO: 10 and SEQ ID NO: 12 (SEQ ID NO:428 and 438).

Such combinations defining functional Fabs with the immuno-specificity herein disclosed, have never been described before.

Particularly preferred Fabs are those comprising the combination of heavy chain variable region SEQ ID NO:2 and light chain variable region SEQ ID NO: 4 (SEQ ID NO:286 and 338); heavy chain SEQ ID NO:10 and light chain SEQ ID NO:12 (SEQ ID NO: 428 and 438).

In this regard, further binding reagents or Fabs with the requested specificity comprise the following heavy and light chain pairing: Heavy Chain SEQ ID NO:18 (SEQ ID NO:416) and Light Chain consisting of SEQ ID NO:30 (SEQ ID NO:444) or SEQ ID NO:32 (SEQ ID NO:442); Heavy Chain SEQ ID NO:20 and Light Chain consisting of SEQ ID NO:32 (SEQ ID NO:442) or SEQ ID NO:34 (SEQ ID NO:450); Heavy Chain SEQ ID NO:22 and Light Chain SEQ ID NO:32 (SEQ ID NO:442); Heavy Chain selected from SEQ ID NO:24 and SEQ ID NO:26 (SEQ ID NO:398) and Light Chain consisting of SEQ ID NO:30 (SEQ ID NO:444); Heavy Chain SEQ ID NO:28 (SEQ ID NO:402) and Light Chain consisting of SEQ ID NO:12 (SEQ ID NO:438), SEQ ID NO:30 (SEQ ID NO:444) or SEQ ID NO:32 (SEQ ID NO:442).

Additional binding reagents with the requested specificity may be obtained by expressing the following variable part of heavy and/or light chains within the proper scaffold: HC SEQ ID NO from 17 to 27 (odd numbers), coding for the aa SEQ ID NO from 18 to 28 (even numbers) and LC SEQ ID NO from 29 to 33 (odd numbers), coding for the aa SEQ ID NO from 30 to 34 (even numbers). Functional fragments of the above heavy and light chains, maintaining the binding activity for both antigens transgelin and ompK36 homologous proteins or fragments thereof, are equally comprised in the present invention. In this regard, since it is known that the most important antigen binding determining regions are complementarity determining regions (CDR) (also termed “minimal recognition units,” or “hypervariable regions”) and in particular CDR3 regions, particularly preferred antibodies are those comprising the following set of CDRs 1-3:

TABLE 1
preferred CDRs sequences

| | | CDR1 | | |
|---|---|---|---|---|
| code | Chain | (SEQ ID NO) | CDR2 | CDR3 |
| Fab | HC2 | GGSIGSGSYS | ISDSGNT | CARGRGILTGLFDYW |
| 7816 | | (SEQ ID NO: 36) | (SEQ ID NO: 38) | (SEQ ID NO: 40) |
| | LC4 | QSVLDNSNHKNS | WAS | CQQYYSTPWTF |
| | | (SEQ ID NO: 42) | | (SEQIDNO: 44) |
| Fab | HC6 | GGSISSSNW | IDHSGTT | CARGAKDNWGFDYW |
| 5LCx | | (SEQ ID NO: 46) | (SEQIDNO: 48) | (SEQ ID NO: 50) |
| | LC8 | QTI | SAT | CQHDYNDPRTF |
| | | | | (SEQ ID NO: 52) |
| Fab | HC10 | GFTFSNGW | IRSNPDGGTT | CITDRGDWKWGVPRDLTYW |
| 1630 | | (SEQ ID NO: 54) | (SEQ ID NO: 56) | (SEQ ID NO: 58) |
| | LC12 | QSVDSNY | GAY | CQQYLSPPITF |
| | | (SEQ ID NO: 60) | | (SEQIDNO: 62) |
| Fab | HC14 | GFTFSDYY | ISSGGDTI | CACRGVW |
| 248 | | (SEQ ID NO: 64) | (SEQ ID NO: 66) | (SEQ ID NO: 68) |
| | LC16 | QSISFH | GTS | CQQYHNWPPLTF |
| | | (SEQ ID NO:70) | | (SEQ ID NO: 79) |

Particularly preferred CDR sets correspond to: set 1) SEQ ID NO: 36, 38 and 40; set 2): SEQ ID NO:54, SEQ ID NO:56 SEQ ID NO:58, for a Heavy chain, and to set 3) SEQ ID NO:42 and 44 and set 4) to SEQ ID NO:60 and 62, for a Light chain. The above CDR sets (comprising CDRs 1, 2 and 3, where present) may provide, once introduced in the correct scaffold, binding reagents which can be used against the antigens transgelin and OmpK36 homologous proteins or, according to a preferred embodiment, antigens in the atherosclerotic plaque.

The term “antibody” therefore further encompasses any antibody format, and includes antibodies or binding agents comprising at least one of the above mentioned HC and LC variable region combinations or functional fragments thereof, such as CDR and CDR sets able to bind specifically the antigens in any genetically engineered or synthetic realization.

Preferred antibody formats according to the invention are either Fab format or the Fc-carrying format, wherein said Fc is preferably an IgG1. In a preferred embodiment, the antibody comprises 2 sets of each preferred HC and LC combination, linked by at least one disulfide bridge. Enzymatic fragments of such preferred formats are also comprised within the present invention.

Antibody formats encompass, just by way of example, the above identified HC, LC variable region combination thereof or CDR sets 1)-4), further comprising: F(ab′)2, Fab, Fab′, Fv, Fc, and Fd fragments, incorporated into the following embodiments: single domain antibodies, single-chain antibodies, maxibodies, minibodies, intrabodies, diabodies, triabodies, tetrabodies, v-NAR and bis-scFv (see e.g. Hollinger and Hudson, Nature Biotechnology, 23(9):1126-1136 (2005)).

Antigen binding fragments derived from an antibody can be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)2. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

Beside CDRs, antibody fragments may also be any synthetic or genetically engineered protein having an amino acid sequence corresponding to those herein disclosed. According to the last embodiment, the invention further discloses the polynucleotide sequences of the variable region of the Heavy and Light chains above identified and corresponding respectively to: SEQ ID NO:1, 5 and 9 (corresponding to SEQ ID NO:285, SEQ ID NO:407 and SEQ ID NO:427, Heavy chains), and SEQ ID NO:3 (SEQ ID NO:337), 7, 11 (SEQ ID NO:437), Light chains). The invention encompasses vectors comprising at least one of each H and L chain variable chain combinations, as follows: SEQ ID NO:1 (SEQ ID NO:285) and 3 (SEQ ID NO:337); SEQ ID NO:5 (SEQ ID NO:407) and SEQ ID NO:7; SEQ ID NO:9 (SEQ ID NO:427) and SEQ ID NO:11 (SEQ ID NO:437), SEQ ID NO:13 (SEQ ID NO:43) and SEQ ID NO:15 (SEQ ID NO:53). Further preferred embodiments are represented by the nucleotides encoding for the following pairs of heavy and light chains SEQ ID NO:17 (SEQ ID NO:415) and SEQ ID NO:29 (SEQ ID NO: 443) or SEQ ID NO:31 (SEQ ID NO:441); SEQ ID NO:19 and SEQ ID NO:31 (SEQ ID NO:441) or SEQ ID NO 33 (SEQ ID NO:449 ; SEQ ID NO 21 and SEQ ID NO:31 (SEQ ID NO:441); SEQ ID NO:23 and SEQ ID NO:29 (SEQ ID NO:443) ; SEQ ID NO:25 (SEQ ID NO:397) and SEQ ID NO:29 (SEQ ID NO:443) ; SEQ ID NO:27 (SEQ ID NO:401) and SEQ ID NO:11 (SEQ ID NO:437) or SEQ ID NO:29 (SEQ ID NO:443) or SEQ ID NO:31 (SEQ ID NO:441) are comprised in the expression vectors to originate the preferred combination of Heavy and Light chain variable regions antibody or fragments thereof, such as Fab' fragments. The term “antibody fragments” further includes isolated fragments consisting of the light chain variable region, “Fv” fragments consisting of the variable regions of the heavy and light chains, and recombinant single chain polypeptide molecules in which light and heavy variable regions are connected by a peptide linker (scFv proteins). Antibody fragments also comprise CDRs of the antibody. CDRs can be obtained by expressing polynucleotides encoding the CDR of interest. Preferred polynucleotide sequences in this regard, are:
SEQ ID NO: 35, 37 and 39
SEQ ID NO: 41 and 43
SEQ ID NO: 53, SEQ ID NO: 55 and SEQ ID NO: 57 and
SEQ ID NO: 59 and 61, encoding the CDRs of interest.

Said polynucleotides are used, for example, in the preparation of antibodies' variable regions (see, for example, Hoogenboorn H. R. et al. Immunol Rev., 1992, 130:41-68). The binding agent may comprise at least two, three, four, five or six CDRs as described herein. The binding agent may comprise at least one variable region domain of an antibody described herein.

The variable region domains of either light and heavy chains may be further engineered by insertions, deletions, or changes in or to the amino acid sequences of the specific antibody.

In this regard, further binding reagents with the requested specificity are obtained by expressing the following variable part of heavy (HC) and/or light chains (LC) within the proper scaffold: HC SEQ ID NO 17 to 27 (odd numbers), coding for the amino acid SEQ ID NO 18 to 28 (even numbers) and LC SEQ ID NO 29 to 33 (odd numbers), coding for the amino acid SEQ ID NO 30 to 34 (even numbers). Preferred heavy and light chains pairs are the following: HC SEQ ID NO 17 to 27 (odd numbers), coding for the amino acid SEQ ID NO 18 to 28 (even numbers) and LC SEQ ID NO 29 to 33 (odd numbers), coding for the amino acid SEQ ID NO 30 to 34 (even numbers). Selected Fab's further comprise the following heavy and light chain pairing wherein the heavy and light chains comprises or consists respectively of: Heavy Chain SEQ ID NO:18 (SEQ ID NO:416) (SEQ ID NO:416) and light chain SEQ ID NO:30 (SEQ ID NO:444) or SEQ ID NO: 32 (SEQ ID NO:442); Heavy Chain SEQ ID NO:20 and Light Chain SEQ ID NO 32 (SEQ ID NO:442) or SEQ ID NO 34 (SEQ ID NO:450); Heavy Chain SEQ ID NO 22 and Light Chain SEQ ID NO 32 (SEQ ID NO:442); Heavy Chain selected from: SEQ ID NO 24 and SEQ ID NO 26 (SEQ ID NO:398) and Light Chain SEQ ID NO 30 (SEQ ID NO:444); Heavy Chain SEQ ID NO 28 (SEQ ID NO:402) and Light Chain SEQ ID NO 12 (SEQ ID NO:438), SEQ ID NO 30 (SEQ ID NO:444) or SEQ ID NO 32 (SEQ ID NO:442).

Engineered antibodies further comprise Fab or Fab' or functional fragments thereof, as above disclosed, covalently attached at a C-terminal amino acid to at least one other antibody domain or a fragment thereof. Thus, for example, a VH domain that is present in the variable region domain may be linked to an immunoglobulin CH1 domain, or a fragment thereof. Similarly a VL domain may be linked to a CK domain or a fragment thereof. In this way, for example, the antibody may be a Fab fragment wherein the antigen binding domain contains associated VH and VL domains covalently linked at their C-termini to a CH1 and CK domain, respectively. The CH1 domain may be extended with further amino acids, for example, to provide a hinge region or a portion of a hinge region domain as found in a Fab' fragment, or to provide further domains, such as antibody CH2 and CH3 domains.

According to a preferred embodiment, the engineered antibody comprises preferred Fabs such as SEQ ID NO: 1 and 3, etc. covalently linked to a human Fc IgG1 region with sequence described in Liang, Met al. J. Immunol. Meth., 2001, 247:119-30.

The DNA encoding an antibody of the invention or fragment thereof may be propagated and expressed according to any of a variety of well-known procedures for nucleic acid excision, ligation, transformation, and transfection using any number of known expression vectors. Thus, in certain embodiments, expression of an antibody fragment may be preferred in a prokaryotic host, such as Escherichia coli (see, e.g., Pluckthun et al, Methods Enzymol, 178:497-515 (1989)). In certain other embodiments, expression of the antibody or a fragment thereof may be preferred in eukaryotic host cells, including yeasts (e.g., Saccharomyces cerevisiae, Schizosaccharomyces pombe, and Pichia pastoris), animal cells (including mammalian cells) or plant cells. Examples of suitable animal cells include, but are not limited to, myeloma (such as a mouse NSO line), COS, CHO, hybridoma cells or insect cells such as Sf9. Examples of plant cells include tobacco, corn, soybean, and rice cells. Particularly preferred eukaryotic cells are insect cells, in particular Sf9 cells.

Furthermore binding agents comprise at least one of the above CDR sets and relevant nucleotide sequences cloned into known antibody framework regions (IgG1, IgG2, etc.) encoding sequences or conjugated to a suitable vehicle to enhance the half-life thereof and biological activity. Suitable vehicles include, but are not limited to Fc, polyethylene glycol (PEG), albumin, transferrin, and the like. These and other suitable vehicles are known in the art.

Consistent with the hypothesis of a molecular mimicry between bacterial antigens sharing high homology with OmpK36 and human SM22, further confirming that this mechanism and the related immunological assays represent important tools for the development of reagents for investigating atherogenesis or even, directly, as diagnostic tools for atherogenic disorders, the Applicants confirmed that some commercial antibodies against human transgelin 1, also bind OmpK36.

Therefore, the invention further extends to the use of antibodies developed against human transgelin 1, preferably anti-TAGLN monoclonal antibodies selected from the group of commercial anti-TAGLN antibodies which cross-react with a bacterial antigen having at least 50% homology with OmpK36, in an immunological assay for identifying TAGLN-1 epitopes important for the development of atherosclerotic specific reagents and/or antigens. Even more preferably said antibodies are AbNova antibodies selected from the group consisting of: Mabs H6876 M06, M03 and M04.

According to this embodiment of the invention, anti-TAGLN Mabs are used to select epitopes derived by TAGLN fragmentation by proteolytic digestion or synthesis of overlapping peptides along the amino acid sequence of the antigen wherein said peptides are obtained either by chemical or enzymatic cleavage or by recombinant DNA techniques, or by peptide synthesis, by an immunoaffinity assay. For instance, said peptides are bound to a solid phase, such as a microplate, or the proteolytic digestion mixture is separated on a polyacrylammide gel and the anti-TAGLN Mab or the human antibodies according to the invention, are used as primary antibodies to recognize the epitope. Suitable secondary, optionally labelled, antibodies are used as revelation antibodies to detect the bound antibody.

The use of the antibodies of the invention is also foreseen for identifying immunologically reacting spots after 2-D separation of biologic samples, from an atherosclerotic plaque for the identification of atherosclerosis related antigens by western blotting.

Competition assays provide for the use of the antibodies of the present invention, human and recombinant and/or monoclonal anti-TAGLN showing cross reactivity with a protein having similarity with OmpK36, by ELISA with TAGLN bound on a solid phase, wherein the binding between the antigen and the antibody is competed by fragments of the antigen and a lowering of the signal corresponds to an effective competition compared to the reaction in the absence of any competitor molecule.

Identification of antibodies (also called immunoglobulins, to be distinguished from the antibodies developed in the present invention) against an atherosclerosis related antigen, developed by patients within the frame of an immune reaction during an atherogenic disorder, is carried out by allowing the unknown biological sample to react with human transgelin 1 or fragments thereof, bound to a solid matrix or dispersed in a liquid phase, optionally in competition with the human recombinant antibodies according to the main aspect of the invention, or with a monoclonal anti-TAGLN antibody as defined above. Positive samples are revealed and/or captured by using secondary antibodies or reagents, optionally labelled.

Engineered variants of binding agents also comprise glycosylation variants wherein the number and/or type of glycosylation site has been altered compared to the amino acid sequences of a parent polypeptide. In certain embodiments, variants comprise a greater or a lesser number of N-linked glycosylation sites than the native protein. A N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines. Desired amino acid substitutions (whether conservative or non-conservative) can be determined by those skilled in the art at the time such substitutions are desired. In certain embodiments, amino acid substitutions can be used to identify important residues of antibodies to the antigens, or to increase or decrease the affinity of the antibodies to the antigens described herein. According to certain embodiments, preferred amino acid substitutions are those which: (1) reduce susceptibility to proteolysis, (2) reduce susceptibility to oxidation, (3) alter binding affinity for forming protein complexes, (4) alter binding affinities, (5) increase cell productivity, and/or (6) confer or modify other physicochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterize the parent sequence).

In certain embodiments, binding agents of the invention may be chemically bonded with polymers, lipids, or other moieties. Particularly preferred moieties are those comprising “contrast imaging agent” or “contrast agent”, used herein interchangeably to provide for an imaging detectable moiety” or, with equivalent meaning, “imaging moiety or moieties”

These terms refer to any moiety detectable by imaging procedures, that is to say any moiety able to provide, to improve or, in any way, to advantageously modify the signal detected by an imaging diagnostic technique today in use. Among them are, for instance, magnetic resonance imaging, radio-imaging, ultrasound imaging, x-ray imaging, light imaging and the like, all of which enable the registration of diagnostically useful, preferably contrasted, images when used in association with the said techniques.

Suitable examples of the said imaging detectable moieties may thus include, for instance, chelated gamma ray or positron emitting radionuclides; paramagnetic metal ions in the form of chelated or polychelated complexes as well as of micellar systems, liposomes and microspheres; magnetic, diamagnetic or superparamagnetic coated particles, microparticles and nanoparticles; hyperpolarized NMR-active nuclei; X-ray absorbing agents including atoms of atomic number higher than 20; bubbles, microbubbles, balloons and microemulsions including biocompatible echogenic gas; reporters suitable for optical imaging including dyes, fluorescent or phosphorescent molecules, molecules absorbing in the UV spectrum, molecules capable of absorption within near or far infrared radiations, a quantum dot and, in general, all moieties which generate a detectable substance.

According to a preferred embodiment of the invention the contrast imaging agents of the invention comprise one or more binding reagents as disclosed in the present invention, with one or more imaging moieties attached to each other, either directly or through any suitable linker.

As such, just as an example, an imaging moiety could be represented by the residue of a known diagnostic agent, for instance of a chelated complex of a paramagnetic metal ion, having formula below:

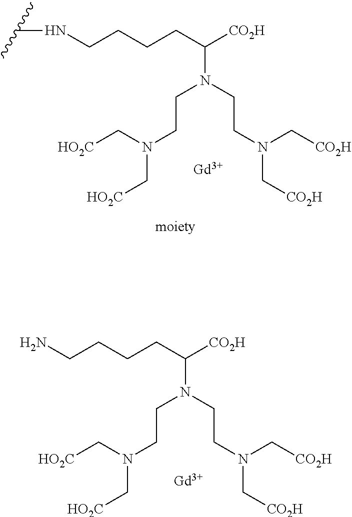

wherein the dotted line just represents the position of attachment of this moiety with the rest of the molecule.

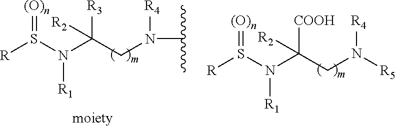

Further, and unless otherwise provided, the term “labelled with” means that the imaging moieties are attached, either directly or through suitable spacers or linkers, to the binding reagents of the present invention.

Materials detectable by diagnostic imaging modalities are known in the art, the imaging modality to be used is selected according to the imaging detectable moiety attached or linked to the binding reagent. To briefly summarize: as far as optical imaging is concerned suitable optically active imaging moieties include, for instance, optical dyes such as organic chromophores or fluorophores, having extensive delocalized ring systems and absorption or emission maxima in the range of 400-1500 nm; fluorescent molecules such as fluorescein; phosphorescent molecules; molecules absorbing in the UV spectrum; a quantum dot (e.g. fluorescent nanocrystals); or molecules capable of absorption of near or far infrared radiations.

Optical parameters to be detected in the preparation of an image may include, as an example, transmitted radiation, absorption, fluorescent or phosphorescent emission, light reflection, changes in absorbance amplitude or maxima, and elastically scattered radiation. For example, the biological tissue is relatively translucent to light in the near infrared (NIR) wavelength range of 650-1000 nm. NIR radiations can penetrate tissues up to several centimetres, permitting the use of the diagnostic agents of the invention comprising a NIR moiety to image target-containing tissues in vivo.

Near infrared dyes may include, for example, cyanine or indocyanine compounds such as, Cy5.5, IRDye800, indocyanine green (ICG) and derivatives thereof, including the tetrasulfonic acid substituted indocyanine green (TS-ICG), and combinations thereof.

In another embodiment, the compounds of the invention may include photolabels, such as optical dyes, including organic chromophores or fluorophores, having extensively conjugated and hence delocalized ring systems and having absorption or emission maxima in the range of 400-1500 nm. The compounds of the invention may alternatively be derivatized with bioluminescent molecules. The preferred range of absorption maxima for photolabels is between 600 and 1000 nm to minimize interference with the signal from hemoglobin. Preferably, photoabsorption labels have large molar absorptivities, e.g. >105 cm-1M-1, while fluorescent optical dyes have high quantum yields. Examples of optical dyes include, but are not limited to, those described in WO 96/23524.

In an embodiment of the invention, the labelling moiety for optical imaging is selected from the group of cyanine, indocyanines, phthalocyanines, naphthocyanines, porphyrins, pyrilium, azulenium or azo-dyes, anthraquinones, naphthoquinones.

Preferably, within this class are fluorescein, 5-carboxyfluorescein, indocyanine green, Cy5, Cy5.5, and derivatives thereof.

The optical imaging agents described above may also be used for acousto-optical or sonoluminescent imaging performed with optically labelled imaging agents according to known methods (see, as an example: WO 98/57666). In acousto-optical imaging, ultrasound radiation is applied to the subject so as to affect the optical parameters of the transmitted, emitted or reflected light. In sonoluminescent imaging, the applied ultrasound actually generates the light detected.

As a preferred example, the above conjugation or labelling may occur between a carboxyl or amino function of the optically active imaging moiety, and the amino or carboxyl function of the binding reagents according to the invention or, optionally, with the ending amino or carboxyl functions of a linker between them.

In any case, any of the functional groups involved in the said conjugation reactions so as to give rise to the imaging agents of the invention are suitably selected in order not to reduce or modify the imaging capability of the optically active agent, nor to impair the affinity of the binding reagents of the invention.

As far as MRI contrast agents are concerned, MRI detectable moieties may comprise the residue of a chelating ligand that is labelled, in its turn, with a paramagnetic metal element detectable by MRI techniques.

Preferred paramagnetic metal elements are those having atomic number ranging between 20 and 31, 39, 42, 43, 44, 49 and between 57 and 83.

More preferred are paramagnetic metal ions selected from the following: Fe(2+), Fe(3+), Cu(2+), Ni(2+), Rh(2+), Co(2+), Cr(3+), Gd(3+), Eu(3+), Dy(3+), Tb(3+), Pm(3+), Nd(3+), Tm(3+), Ce(3+), Y(3+), Ho(3+), Er(3+), La(3+), Yb(3+), Mn(3+), Mn(2+); Gd(3+) being the most preferred one.

With the term “chelator”, “chelating ligand” or “chelating agent”, as used herein interchangeably, we intend chemical moieties, agents, compounds or molecules characterized by the presence of polar groups able to a form a complex containing more than one coordinated bond with a transition metal or another metal entity. In a preferred aspect of the invention the said chelating ligand includes cyclic or linear polyamino, polycarboxylic or polyphosphonic acids. The said ligands comprise, in addition, groups that allow for the conjugation (i.e. labelling) with the rest of the molecule. Typically, the said groups include thiol, amino or carboxyl functions either present as such or as optionally activated functions.

For MRI purposes, the chelating ligands are in their turn labelled with the selected paramagnetic metal, so as to form a chelate or coordinate complex with that metal.

Suitable chelating ligands include those selected from the group consisting of: polyaminopolycarboxylic acids and derivative thereof comprising, for example, diethylenetriamine pentaacetic acid (DTPA), benzo DTPA, dibenzo DTPA, phenyl DTPA, diphenyl DTPA, benzyl DTPA, dibenzyl DTPA, N,N-Bis [2-[(carboxymethyl)[(methylcarbamoyl)methyl]ethyl]-glycine (DTPA-BMA), N-[2-[bis(carboxymethyl)amino]-3-(4-ethoxyphenyl)propyl)]-N-[2-[bis(carboxy-methyl)amino]ethyl]glycine (EOB-DTPA), 4-carboxy-5,8,11-tris(carboxymethyl)-1-phenyl-2-oxa-5,8,11-triazatridecan-13-oic acid (BOPTA), N,N-bis[2-[bis(carboxymethyl)amino]ethyl]L-glutamic acid (DTPA-Glu) and DTPA-Lys; ethylenediaminotetraacetic acid (EDTA); 1,4,7,10-teraazacyclododecane-1,4,7,-triacetic acid (DO3A) and derivatives thereof including, for example, [10-(2-hydroxypropyl)-1,4,7,10-teraazacyclododecane-1,4,7,-triacetic acid (HPDO3A); 1,4,7-triazacyclononane-N,N′,N″-triacetic acid (NOTA); 6-[bis(carboxymethyl) amino]tetrahydro-6-methyl-1H-1,4-diazepine-1,4(5H)-diacetic acid (AAZTA) and derivative thereof, for instance including those disclosed in WO 03/008390, 1,4,7,10 tetraazacyclotetradecane-

1,4,7,10 tetraacetic acid (DOTA) and derivatives thereof including, for instance, benzo-DOTA, dibenzo-DOTA, (α,α′, α″,α′″)-tetramethyl-1,4,7,10 tetraazacyclotetradecane 1,4,7,10 tetraacetic acid (DOTMA); and 1,4,8,11l -tetraazacyclotetradecane-N,N′,N″,N′″-tetraacetic acid (TETA); or corresponding compounds wherein one or more of the carboxylic groups is replaced by a phosphonic and/or phosphinic group including, for instance, N,N′-bis-(pyridoxal-5-phosphate)-ethylenediamine-N.N′-diacetic acid (DPDP); ethylenedinitrilotetrakis(methylphosphonic) acid (EDTP), 1,4,7,10 tetraazacyclotetradecane 1,4,7,10 tetra(methylenephosphonic) acid (DOTP), the phosphonoalkyl-polyaza macrocyclic compounds disclosed in U.S. Pat. No. 5,362,476 and U.S. Pat. No. 5,409,689; the linear phosphonoalkyl derivatives disclosed in U.S. Pat. No. 6,509,324; as well as macrocyclic chelants such as texaphirines, porphyrins and phthalocyanines.

As far as Nuclear Imaging (Radionuclide Imaging) moieties detectable by imaging techniques known in the art such as, for instance, scintigraphic imaging, Single Photon Emission Computed Tomography (SPECT) and Positron Emission Tomography (PET) may comprise the residue of a chelating agent or ligand labelled with a radionuclide detectable by the above scintigraphic, SPECT or PET imaging techniques. Suitable chelating ligands are those above reported for MRI imaging techniques and further include linear or macrocyclic ligands purposely intended for radionuclides.

Preferred metal radionuclides for scintigraphy, PET or radiotherapy include: ⁹⁹mTc, ⁵¹Cr, ⁶⁷Ga, ⁶⁸Ga, ⁴⁷Sc, ⁵¹Cr, ¹⁶⁷Tm, ¹⁴¹Ce, ¹¹¹In, ¹⁶⁸Yb, ¹⁷⁴Yb, ¹⁴⁰La, ⁹⁰Y, ^{88l Y,} ¹⁵³Sm, ¹⁶⁶Ho, ¹⁶⁵Dy, ¹⁶⁶Dy, ⁶²Cu,⁶⁴Cu, ⁹⁷Ru, ¹⁰³Ru, ¹⁸⁶Re, ¹⁸⁸Re, ²⁰³Pn, ²¹¹Bi, ²¹²Bi, ²¹³Bi, ²¹⁴Bi, ¹⁰⁵Rh, ¹⁰⁹Pd, ^{177m}Sn, ¹⁴⁹Pm, ¹⁶¹Tb, ¹⁷⁷Lu, ¹⁹⁸Au and ¹⁹⁹Au and oxides or nitrides thereof. The choice of metal will be determined based on the desired therapeutic or diagnostic application. For example, for diagnostic purposes (e.g., to diagnose and monitor therapeutic progress in primary tumors and metastases), the preferred radionuclides include ⁶⁴Cu, ⁶⁷Ga, ⁶⁸Ga, ⁹⁹mTc, and ¹¹¹In, with ^{99m}Tc, ^{111In} and ⁶⁸Ga being especially preferred.

^{99m}Tc is particularly useful and is a preferred for diagnostic radionuclide for SPECT and planar imaging because of its low cost, availability, imaging properties, and high specific activity. The nuclear and radioactive properties of 99mTc make this isotope an ideal scintigraphic imaging agent. This isotope has a single photon energy of 140 keV and a radioactive half life of about 6 hours, and is readily available from a 99Mo 99mTc generator. For example, the ^{99m}Tc labeled peptide can be used to diagnose and monitor therapeutic progress in primary tumors and metastases. Likewise, 68Ga is particularly useful as it is an ideal isotope for positron emission tomography (PET). It is produced from a ⁶⁸Germanium/⁶⁸Gallium generator, thus allowing the use of a positron-emitting isotope without access to a cyclotron. Several types of 68Ge/68Ga generators are known to those skilled in the art. These differ in the nature of the adsorbant used to retain ⁶⁸Ge, the long-lived parent isotope, on the generator and the eluant used to elute the ⁶⁸Ga off of the column (see e.g. Fania et al, Contrast Media Mol. Imaging 2008, 3 67-77; Zhernosekov et al. J. Nucl. Med, 2007, 48, 1741-1748).

Therefore, the present invention also relates to agents for SPECT or PET imaging techniques comprising one or more residues of the binding reagents described above labelled with one or more moieties that are, in their turn, labelled with halogen radionuclides.

Means of conjugation or labelling between the binding reagents and the radioimaging detectable moiety, either directly or through a suitable linker, have been already described above for MRI agents.

According to an additional embodiment of the invention, the imaging moiety enables the formation of liposomes, microbubbles, microballoons, microspheres or emulsions and is preferably selected from the group consisting of: surfactants, sphingolipids, oligolipids, phospholipids, proteins, polypeptides, carbohydrates, synthetic or natural polymeric materials and mixtures thereof as Ultrasound contrast agents.

Preferably, the binding reagents as disclosed, comprise a residue labelled with a lipidic or phospholipidic component enabling the formation of the above liposomes, microbubbles, microballoons, microspheres or emulsions.

Interestingly, as said liposomes are formed according to conventional techniques by properly agitating these latter compounds, the liposomes thus formed will comprise, on their surface, a high number of the binding reagents according to the invention linked with the suitable moiety.

A further embodiment of the invention is thus represented by an ultrasound contrast agent in the form of liposomes, microbubbles, microballoons, microspheres or even emulsions, containing a material capable of generating an echogenic gas, further labelled with a plurality of the binding reagents according to the invention.

In the present description, and unless otherwise provided, with the term “lipid”, “phospholipid” or “lipidic/phospholipidic component”, as used herein, we intend a synthetic or naturally-occurring amphipatic compound which comprises a hydrophilic component and a hydrophobic component. Lipids include, for example, fatty acids, neutral fats, phosphatides, glycolipids, aliphatic alcohols and waxes, terpenes and steroids.

Examples of suitable lipids according to the invention include: phosphatidylcholines such as dioleoylphosphatidylcholine, dimyristoylphosphatidylcholine, dipalmitoyl-phosphatidylcholine and diasteroylphosphatidylcholine; phosphatidylethanolamines such as dipalmitoylphosphatidylethanolamine, dioleoylphosphatidylethanolamine and N-succinil-dioleoylphosphatidyl-ethanolamine; phosphatidylserine; dipalmitoylphosphatidylserine; phosphatidylglycerols; sphingolipids; glycolipids such as ganglioside GM1; glucolipids; sulphatides; phosphatidic acid and derivatives such as dipalmitoyl phosphatidic acid (DPPA); fatty acids including palmitic, stearic, arachidonic, lauric, myristic, lauroleic, physeteric, myristoleic, palmitoleic, petroselinic, oleic, isolauric, isomyristic and isostearic fatty acids; cholesterol and derivatives such as cholesterol hemisuccinate or sulphate and cholesteryl-(4-trimethylammonio)-butanoate; polyoxyethylene fatty acids esters, alcohols or alcohol ethers; polyoxyethylated sorbitan fatty acid esters, glycerol polyethylene glycol oxy-stearate; glycerol polyethylene glycol ricinoleate; ethoxylated soybean sterols; ethoxylated castor oil; polyoxyethylene polyoxypropylene fatty acid polymers; polyoxyethylene fatty acid stearates; 1,2-dioleoyl-sn-glycerol; 1,2-dipalmitoyl-sn-3-succinylglycerol; 1,3-dipalmitoyl-2-succinylglycerol; 1-hexadecyl-2-palmitoyl-glycerophosphoethanolamine; N-succinyl-dioctadecylamine; palmitoylhomocysteine; lauryltrimethylammonium bromide; cetyltrimethyl-ammonium bromide; myristyltrimethylammonium bromide; alkyldimethylbenzylammonium chloride wherein alkyl is a C₁₂, C₁₄ or C₁₆ alkyl; benzyldimethyldodecylammonium bromide; benzyldimethyldodecyl ammonium chloride; benzyldimethylhexadecylammonium bromide; benzyldimethylhexadecylammonium chloride; benzyldimethyltetradecyl ammonium bromide; benzyldimethyltetradecyl ammonium chloride; cetyldimethylethylammonium chloride; cetylpyridinium bromide; cetylpyridinium chloride; N-[1,2,3-dioleoyloxy)-propyl]-N,N,N-trimethylammonium chloride (DOTMA); 1,2-dioleoyloxy-3-(trimethylammonium)propane (DOTAP); and 1,2-dioleoyl-c-(4′-trimethylammonium)-butanoyl-sn-glycerol (DOTB).

With the term “liposomes”, as used herein, we refer to a generally spherical cluster or aggregate of amphipathic compounds, including lipid/phospholipid compounds, typically in the form of one or more concentric layers, for example bilayers. They may also be referred to herein as lipid vesicles.

With the term “vesicle”, as used herein, we refer to a spherical entity which is characterized by the presence of an internal void. Preferred vesicles are formulated from lipids, including the various lipids described herein and, in any given vesicle, the lipids may be in the form of monolayer or bilayer.

The lipid vesicles described herein include such entities commonly referred to as liposomes, micelles, bubbles, microbubbles, microspheres and the like. The internal void of the vesicles may be filled with a gas or a gaseous precursor.

The term “bubbles”, as used herein, refers to a vesicle which is generally characterized by the presence of one or more membranes or walls surrounding an internal void that is filled with a gas or a gas precursor.

The terms “microspheres” and “microballoons”, as used herein, preferably refer to spheres having a diameter of less than, or equal to, 10 microns.

These microballoons have an envelope including a biodegradable physiologically compatible polymer or a biodegradable solid lipid. The polymers useful for the preparation of the microballoons of the present invention can be selected from the biodegradable physiologically compatible polymers such as any of those described in: EP 458745.

Polymer can be selected from biodegradable physiologically compatible polymers, such as polysaccharides of low water solubility, polylactides and polyglycolides and their copolymers, copolymers of lactides and lactones such as 68 -caprolactone, γ-valerolactone and polypeptides.

The microballoons of the present invention can also be prepared according to the methods disclosed in WO 96/15815, where the microballoons are made from a biodegradable membrane comprising biodegradable lipids, preferably selected from mono- di-, tri-glycerides, fatty acids, sterols, waxes and mixtures thereof. Preferred lipids are di- or tri-glycerides, e.g. di- or tri-myristin, -palmityn or -stearin, in particular tripalmitin or tristearin.

The microballoons may employ any of the gases disclosed herein or known to the skilled artisan for ultrasound techniques.

Any biocompatible gas may be used in the vesicular contrast agents of the invention. The term “gas”, as used herein, includes any substance (comprehensive of mixtures thereof) substantially in gaseous form at the normal human body temperature.

Said gas may thus include, for example, air, nitrogen, oxygen, CO₂, argon, xenon, krypton, fluorinated gases (including, for example, perfluorocarbons, SF6 or SeF6) and low molecular weight hydrocarbons (for instance those containing from 1 to 7 carbon atoms including alkanes such as methane, ethane, propane, butane or pentane; cycloalkanes such as cyclopropane, cyclobutane or cyclopentane; alkenes or alkynes such as ethylene, propene, propadiene, butene, acetylene, propyne, and/or mixtures thereof). Fluorinated gases are however preferred.

Fluorinated gases include materials which contain at least one fluorine atom. Examples include, but are not limited to, compounds such as SF6, freons (organic compounds containing one or more carbon atoms and fluorine such as CF4, C2F6, C3F8, C4F8, C4F10, CBrF3, CCl2F2, C2CIF5 and CBrCIF2) and perfluorocarbons. The term “perfluorocarbon” refers to compounds containing only carbon and fluorine atoms and include saturated, unsaturated and cyclic perfluorocarbons.

The saturated perfluorocarbons, which are preferred, have the formula CnFn+2, where n is from 1 to 12, preferably from 2 to 10, more preferably from 3 to 8 and even more preferably from 3 to 6. Suitable perfluorocarbons thus include, but are not limited to, CF4, C2F6, C3F8, C4F8, C4F10, C5F12, C6F12, C7F14, C8F18 and C9F20. More preferably, the gas or gas mixture comprises SF6 or a perfluorocarbon selected from the group consisting of: C3F8, C4F8, C4F10, C5F12, C6F12, C7F14, C8F18 with C4F10 being particularly preferred.

As an additional embodiment of the invention the above liposomes, micellar systems, vesicles, microspheres or microballoons, may entrap other imaging moieties among those previously disclosed as further macromolecular aggregates embodiment for diagnostic imaging.

We thus refer, according to an additional embodiment of the invention, to a macromolecular system for use in MRI imaging techniques comprising the above liposomes, micellar systems, vesicles, microspheres or microballoons, being prepared according to conventional methods by starting from the compounds comprising a binding reagent as above disclosed, properly labelled with a lipidic or phospholipidic component as set forth above, and wherein within the cavity of the said liposomes, micellar systems, vesicles, microspheres or microballoons, there are incorporated suitably chelated MRI paramagnetic metal ions.

Preferably, an additional object of the invention is thus represented by the liposomes obtained labelling the binding reagents with lipidic or phospholipidic components, and wherein the inner cavity of the said liposomes comprises the aforementioned chelate complexes of Gd3+ ions or chelated ligands of lanthanide ions for MRI purposes.

Said liposomial imaging agents are characterized by an enhanced sensitivity over traditional MRI contrast agents as they may take advantage of the difference in NMR (Nuclear Magnetic Resonance) signal intensity of water protons in the presence and in the absence of the contrast agent, when a radiofrequency is applied, the said radiofrequency corresponding to the resonance frequence of water protons exchangeable by the system, that is inside and outside the liposomial vesicle.

Such contrast amplification technique is better known as Chemical Exchange Saturation Transfer (CEST) and the materials suitable for the said technology are better known as LIPOCEST, hence of chelated complexes of lanthanide ions entrapped within liposomial vescicles that, according to the present invention, are labelled with a plurality of the binding reagents according to the invention.

In the present invention, the CEST imaging system is represented by a liposomal system. In this case, the chemical shift of the intraliposomal water protons which must be irradiated to observe saturation transfer has been suitably “shifted” as a result of their interaction with a paramagnetic chelate containing a lanthanide metal ion.

The paramagnetic complex can be encapsulated in the aqueous cavity of the liposome (if hydrophilic), and/or incorporated in the lipidic bilayer of the membrane (if amphiphilic).

For a general reference to CEST techniques and LIPOCEST(s) see, as an example, Angew. Chem. Int. Ed. 2007, 46, 966-968; and Chem. Commun., 2008, 600-602.

All the above variants and diagnostic derivatives are deemed to be comprised in the present invention, provided that their affinity to the antigens transgelin and OmpK36 homologous proteins is not significantly altered, with respect to the preferred binding reagent embodiment.

Immunoaffinity to transgelin 1 (Accession Q01995, GI48255907) is usually maintained for protein having at least 80%, more preferably at least 82%, 84%, 86%, 88%, 90%, 92%, 94%, 96%, 98%, 99% sequence similarity with the human transgelin 1 sequence.

The same is true for antibodies binding OmpK36 (Klebsiella OmpK36 GI:295881594 also referred to as SEQ ID NO:76) even though with a lower degree of homology (based on amino acid similarity) with the OmpK36 protein itself, maintained for protein having at least 50% preferably at least 60%, 70%, 80% 82%, 84%, 86%, 88%, 90%, 92%, 94%, 96%, 98%, 99% sequence similarity to the OmpK36 sequence; where for sequence similarity, intermediate values are also comprised within the scope of the invention. By similarity the Applicant means protein-protein primary structure comparison based on both amino acid identity and similarity as defined for example in: A Structural Basis of Sequence Comparisons An evaluation of scoring methodologies Johnson, M. S., Overington, J. P. 1993 Journal of Molecular Biology 233: 716-738 or: Improved tools for biological sequence comparison. Pearson, W. R., Lipman, D. J. 1988 Proceedings of the National Academy of Sciences USA 85:2444-2448; or: Searching Protein Sequence Libraries: Comparison of the Sensitivity and Selectivity of the Smith Waterman and FASTA algorithms. Pearson, W. R. 1991 Genomics 11:635-650. Other examples of sequence similarity are based on the similarity of amino acids used to evaluate aa conservative substitutions (as above provided).

Antibodies which are particularly preferred are those able to immunologically recognize transgelin and the outer membrane protein of either Proteus vulgaris or Klebsiella, even more preferably transgelin 1 and Klebsiella OmpK36, by immunoassay, whether performed at the same time or different or in a sequential order.

According to an alternative embodiment the antigens recognized by the present antibodies are transgelin fragments, preferably obtained by synthesis, selected in the group consisting of: N-terminal fragments, comprising amino acids (“aa”) 5-18 with reference to the numbering of the transgelin 1 sequence in the database (Accession Q01995, GI48255907), even more preferably aa 3-20 or even more preferably aa 1-30; C-terminal fragments, comprising aa 185-198, even more preferably comprising aa 170-199, or even more preferably comprising aa 160-201 according to the numbering of the sequence in the reference database.

For OmpK36 homologous proteins, an embodiment alternative to the use of the whole protein, either native or recombinant, is the use of peptides selected in the group consisting of: the OmpK36 N-terminal region comprising aa 1-20; a fragment comprising aa 70-80, or even more preferably comprising aa 68-90; a fragment comprising aa 130-155, or even more preferably comprising aa 125-160; a fragment comprising aa 250-290, preferably comprising aa 263-276. These peptides from both transgelin and OmpK 36, are reagents useful for screening antibodies according to the present invention and are therefore also comprised within its scope.

Beside the above identified isolated antigens or fragments thereof, even more preferably, the binding reagents of the present invention bind in vitro biopsies of the atherosclerotic plaque, more preferably taken from a coronary plaque, as it can be shown by immunoassays on plaque tissue lysate, i.e. by immunoprecipitation or western blot, or by immunohistology on plaque sections.

Without being bound to a particular hypothesis, it is plausible that antibodies against an antigen sharing epitope(s) with either transgelin or Outer membrane proteins homologous to the OmpK36 from Klebsiella, develop as an immune or autoimmune response triggered by inflammation within the vessels, or in other words, as a response to the well characterized atherogenic mechanisms.

Therefore according to a further embodiment, the present invention relates to a method for preparing anti-idiotipic antibodies, where the binding reagents herein disclosed are used as a molecular image of the “atherogenic” antigen. The preparation of anti-idiotipic antibodies is well known to the skilled person and is described for example in Burioni R, et al. PLoS One. 2008;3(10):e3423. Anti-idiotipic antibodies may represent a vaccine protecting from the development of atherosclerosis.

The present invention further relates to a method for the in vitro or in vivo immunodetection of a sample from an atherosclerotic plaque or from human serum, where the antibodies according to the present invention are used Immunoassays take a variety of forms which are all comprised within the scope of the present invention.

Conventional assays may be carried out by ELISA, Western blot, immunoprecipitation, immunofluorescence, immunochemistry or FACS. Further, immunoassays according to the invention may be fluorescent immunoassays, chemiluminescent assays, agglutination assays, nephelometric assays, turbidimetric assays, Western Blots, competitive or non-competitive immunoassay, homogenous or heterogenous immunoassays, and reporter-assays, e.g. a luciferase assay. Reference may be made to any manual such as: “Current Protocols in Immunology”, 1994 ed.

According to a preferred embodiment, the immunoassay is an ELISA or a Western-blot where atherosclerotic plaque lysate is used as a preferred capture antigen.

Furthermore and according to a preferred embodiment, the immunoassay provides for the detection in a sample, of immunoglobulins binding the antigens: transgelin or fragment, variant or derivative thereof and proteins homologous to OmpK36, either at the same time or in succession, preferably by using the antibody of the present invention as competitive binding agents, where the presence of antibodies in an unknown biological sample, i.e. a patient's serum, preferably competing with those according to the present invention, are indicative that an atherogenic process has been triggered and/or is developing or that an atheromatous disease is in course, or that the patient is at risk of developing ACS (Acute Coronary Syndrome).

These antibodies may optionally further bind to histological sections of atherosclerotic plaques already in or developing in arterial vessels, namely coronary or carotid.

Even though these assays may be realized with several technical variants comprised within the scope of the present invention, one of the preferred embodiment is by competitive assays, where at least one of the antibodies according to the present invention is used as a competing reagent with the antibodies in the biological sample in binding to transgelin and OmpK36.

An example of immunoassay realization comprises contacting the antigens with an antibody or fragment or derivative thereof according to the invention and detecting the level of a complex comprising said antibody and transgelin and between said antibody and proteins homologous to OmpK36, variant or derivative thereof in the presence or absence of the sample.

As discussed above, any suitable technique for determining formation of the complex or, by reverse, inhibition of complex formation may be used.

Competitive immunoassays include but are not limited to: radioimmunoassays (RIAs), enzyme-linked immunosorbent assays (ELISAs) and immunochromatographic techniques (ICTs), Western blotting which are well known to those skilled in the art. It will be understood that the present invention encompasses qualitative and quantitative immunoassays.

Suitable immunoassay techniques include both single-site and two-site assays of the non-competitive types, as well as the traditional competitive binding assays. These assays also include direct binding of a labelled antigen-binding molecule to a target antigen wherein said target antigen is transgelin or fragments thereof and proteins homologous to OmpK36 or fragments thereof.

Two-site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by measuring or detecting a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent.

For quantitative data, and as a general practice, positive and negative controls may be used, as known by the skilled man. Positive standards may comprise the human recombinant protein transgelin (either flagged or not) and antibodies specific against it (or the flag), which are both commercially available. OmpK36 from Klebsiella can be easily produced by standard recombinant techniques, starting from the nucleotide sequence encoding for it.

In the typical forward assay, a first antibody having specificity for the antigen or antigenic parts thereof is either covalently or passively bound to a solid surface. The solid surface, also called matrix or support, is typically glass or a polymer, the most commonly used polymers being cellulose, polyacrylamide, nylon, polystyrene, polyvinyl chloride or polypropylene. The solid supports-may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilized first antibody.

An alternative method involves immobilizing the antigen in the biological sample and then exposing the immobilized antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody, is exposed to the target-first antibody complex to form a target-first antibody-second antibody tertiary complex.

The complex is detected by the signal emitted by a reporter molecule. Suitable reporter molecule associated with the antigen-binding molecule may include the following: (a) direct attachment of the reporter molecule to the antibody; (b) indirect attachment of the reporter molecule to the antibody; i. e., attachment of the reporter molecule to another assay reagent which subsequently binds to the antibody; and (c) attachment to a subsequent reaction product of the antibody.

The reporter molecule may be selected from a group including a chromogen, a catalyst, an enzyme, a fluorochrome, a chemiluminescent molecule, a paramagnetic ion, a lanthanide ion such as Europium (Eu), a radioisotope including other nuclear tags and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules include alkaline phosphatase, horseradish peroxidase, luciferase, P-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al.: WO 93/06121.

In the case of an enzyme immunoassay, an enzyme is conjugated to the second antibody, generally by means of glutaraldehyde or periodate. As will be readily recognized, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex, allowed to bind, and then the excess reagent washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium may be chemically coupled to antibodies without altering their binding capacity. When activated by illumination with light of a particular wavelength, the fluorochrome-labelled antibody adsorbs the light energy, inducing an excited state in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest Immunofluorometric assays (IFMA) are well established in the art and are particularly useful for the present method.

For solid phase assays, suitable solid support materials are nitrocellulose, polyvinylchloride or polystyrene, e.g. the well of a microtiter plate.

The invention also provides a diagnostic kit comprising at least one anti-transgelin and OmpK36 binding reagent according to the present invention. The binding reagent is preferably a Fab-IgG1 as disclosed above. In addition, such a kit may optionally comprise one or more of the following:
(1) instructions for using the one or more binding agent(s) for screening, diagnosis, prognosis, therapeutic monitoring or any combination of these applications;
(2) a labeled binding partner to the binding agent(s) of the invention; (3) a solid phase (such as a reagent strip) upon which the binding agent(s) is immobilized; and
(4) a label or insert indicating regulatory approval for screening, diagnostic, prognostic or therapeutic use or any combination thereof.

If no labeled binding partner to the binding agent(s) is provided, the binding agent(s) itself can be labeled with one or more of a detectable marker(s), e.g., a chemiluminescent, enzymatic, fluorescent, or radioactive moiety.

According to a further aspect, the invention provides for pharmaceutical compositions, as injectable formulations for diagnostic purposes, comprising one of the above-described binding reagents along with a pharmaceutically or physiologically acceptable carrier, excipient, or diluent. The development of suitable dosing and treatment regimens for using the particular compositions described herein in a variety of treatment regimens, including e.g., subcutaneous, oral, parenteral, intravenous, intranasal, and intramuscular administration and formulation, is well known in the art, some of which are briefly discussed below for general purposes of illustration. In certain circumstances it will be desirable to deliver the pharmaceutical compositions disclosed herein subcutaneously, parenterally, intravenously, intramuscularly, or intra-peritoneally. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Moreover, for human administration, preparations will preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically- acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles. The formation and use of liposome and liposome-like preparations as potential drug carriers is generally known to those of skill in the art (see for example, Lasic, Trends Biotechnol, 16(7):307-21 (1998); Takakura, Nippon Rinsho, 56(3):691-95 (1998); The use of liposomes does not appear to be associated with autoimmune responses or unacceptable toxicity after systemic delivery. In certain embodiments, liposomes are formed from phospholipids that are dispersed in an aqueous medium and spontaneously form multilamellar concentric bilayer vesicles (also termed multilamellar vesicles (MLVs)).

According to further aspects the invention provides for the use of the binding reagents of the invention as diagnostic tools in the early detection of atherogenic diseases wherein said term comprises an atherogenic ischemic or occlusive evolution in an arterial vessel, further comprising Acute Coronary syndrome and related cardiovascular diseases such as unstable angina, ST Elevation Myocardial Infarction (STEMI), nonSTEMI myocardial infarction) as well as intra-cerebral occlusive disease (STROKE) or peripheral artery occlusive diseases.

In summary, the invention provides for the following embodiments covering detection methods, preferably immuno-detection methods, even more preferably Western-blot and ELISA assays, either:

a) for the detection of antibodies or immunoglobulins developed by a cardiovascular patient against an antigen/(epitope) in an atherosclerotic plaque, said method comprising allowing an unknown biological sample to react with human transgelin-1 or fragments thereof, optionally in competition with an antibody comprising a combination of at least a heavy chain selected from the group consisting of: SEQ ID NO: 2 (SEQ ID NO:286), 6 (SEQ ID NO:408), 10 (SEQ ID NO:428) 14 (SEQ ID NO:44), and at least a light chain selected from the group consisting of: SEQ ID NO: 4 (SEQ ID NO:338), SEQ ID NO:8, SEQ ID NO: 12 (SEQ ID NO:438), SEQ ID NO:16 (SEQ ID NO:54). According to a preferred embodiment, the method comprises using the following heavy and light chain combinations: SEQ ID NO: 2 and SEQ ID NO: 4 (SEQ ID NO:286 and SEQ ID NO:338); SEQ ID NO: 6 (SEQ ID NO:408) and SEQ ID NO: 8; SEQ ID NO: 10 and SEQ ID NO: 12 (SEQ ID NO:428 and SEQ ID NO:438); SEQ ID NO: 14 and SEQ ID NO: 16 (SEQ ID NO:44 and SEQ ID NO:54), or according to a further embodiment of the invention the following heavy and light chain preferred pairs:

Heavy Chain SEQ ID NO:18 (SEQ ID NO:416) (SEQ ID NO:416) and light chain SEQ ID NO:30 (SEQ ID NO:444) or SEQ ID NO: 32 (SEQ ID NO:442); Heavy Chain SEQ ID NO:20 and Light Chain SEQ ID NO 32 (SEQ ID NO:442) or SEQ ID NO 34 (SEQ ID NO:450); Heavy Chain SEQ ID NO 22 and Light Chain SEQ ID NO 32 (SEQ ID NO:442; Heavy Chain selected from: SEWN( )24 and SEQ ID NO 26 (SEQ ID NO:398) and Light Chain SEQ ID NO 30 (SEQ ID NO:444); Heavy Chain SEQ ID NO 28 (SEQ ID NO:402) and Light Chain SEQ ID NO 12 (SEQ ID NO:438), SEQ ID NO 30 (SEQ ID NO:444) or SEQ ID NO 32 (SEQ ID NO:442) as competing antibodies.

The method further comprises allowing said unknown biological sample to react with a protein homologous to OmpK36 or fragments thereof, as described above. The method further provides to define as positive the biological sample where a specific binding to both antigens can be detected. In this regard the order in which said antigens are tested, represents only an indication or a preferred embodiment; or

b) for the detection of an antigen/(epitope) in the atherosclerotic plaque of an unknown sample,

said method comprising allowing said unknown biological sample to react with an antibody comprising at least a heavy chain variable region selected from: SEQ ID NO: 2, 6, 10, 14 and at least a light chain selected from the group consisting of: SEQ ID NO: 4, 8, 12, 16.

Particularly preferred heavy and light chain variable region combinations are the following: SEQ ID NO:2 and SEQ ID NO:4 (SEQ ID NO:286 and 338), SEQ ID NO: 6 (SEQ ID NO:408) and SEQ ID NO: 8; SEQ ID NO: 10 and SEQ ID NO: 12 (SEQ ID NO 428 and 438).

Additional binding reagents are represented by the following pairs of Fab′ variable regions wherein the heavy and light chains comprises or consists respectively of: Heavy Chain SEQ ID NO:18 (SEQ ID NO:416) (SEQ ID NO:416) and light chain SEQ ID NO:30 (SEQ ID NO:444) or SEQ ID NO: 32 (SEQ ID NO:442); Heavy Chain SEQ ID NO:20 and Light Chain SEQ ID NO 32 (SEQ ID NO:442) or SEQ ID NO 34 (SEQ ID NO:450); Heavy Chain SEQ ID NO 22 and Light Chain SEQ ID NO 32 (SEQ ID NO:442; Heavy Chain selected from: SEQ ID NO 24 and SEQ ID NO 26 (SEQ ID NO:398) and Light Chain SEQ ID NO 30 (SEQ ID NO:444); Heavy Chain SEQ ID NO 28 (SEQ ID NO:402) and Light Chain SEQ ID NO 12 (SEQ ID NO:438), SEQ ID NO 30 (SEQ ID NO:444) or SEQ ID NO 32 (SEQ ID NO:442).

The method further provides to define as positive the biological sample where a specific binding to a region of the atherosclerotic plaque can be detected.

Immunodetection methods based on the binding reagents herein disclosed allows the detection of an atherogenic process or ateromatous diseases, wherein said term comprises an atherogenic ischemic or occlusive evolution in an arterial vessel, further comprises Acute Coronary syndrome comprising unstable angina, ST Elevation Myocardial Infarction (STEMI), nonSTEMI myocardial infarction) and related cardiovascular diseases, as well as intra-cerebral occlusive disease (i.e. stroke), peripheral artery occlusive diseases or non-acute coronary diseases.

Furthermore, the invention comprises transgelin, preferably transgelin-1, as a marker of the presence of an atherosclerotic plaque in immunodetection methods, that according to a preferred embodiment, further comprise the detection of such a binding specificity in combination with the detection of a binding specificity to a bacterial outer membrane protein selected among those with at least 50% similarity to OmpK36, or preferably on OmpK36 itself, as defined above.

The invention further provides a method for screening polypeptide libraries, wherein the term polypeptide comprises peptides (3-50 aa long, even cyclic and polypeptides more than 50aa long) and antibodies or fragments thereof, where the panning is sequentially carried out both on a bacterial outer membrane protein, selected among those with at least 50% similarity to OmpK36, as defined above, or preferably on OmpK36 itself, and on SM22 (TAGLN or transgelin-1) in any order. According to a preferred embodiment, the affinity selection (or panning) is carried out first on SM22 (TAGLN or transgelin-1) and then on a protein selected among those with at least 50% similarity to OmpK36, as defined above, or preferably on OmpK36 itself or fragments thereof.

One or more rounds of panning on both proteins, each at a time, are preferably carried out.

The antigen(s) is immobilized on a solid phase, such as on an ELISA microplate or in a tube, or is in solution.

A specific binding reducing means such as BSA or milk powder or other macromolecules, such as PEG, dextran or similar are used, preferably milk powder.

Human antibody libraries either naïve or antigen-primed are known. Preferred antibody libraries are human and are antigen primed. Human libraries from atherosclerotic lesions, where a local immune response (see also Burioni R. et al. J Immunol., 2009, 183:2537-2544) has been demonstrated to occur in patients suffering from Acute Coronary Syndrome, are even more preferred. Libraries are preferably phage-displayed as disclosed in WO2009/037297 or sublibraries thereof. The preparation of libraries from an atherosclerotic sample is also contemplated and feasible according to the instructions i.e. in Manual Phage Display. A laboratory manual, C. Barbas, D. Burton, J. Scott, G. Silvermann January 2001 ed, CSH Press.

Known methods are known to the skilled man for the selection of antibody or binding reagents displaying clones with the highest immuno-affinity to the antigen(s). Identification of the nucleotide sequence encoding the selected antibody or fragment thereof, after the selection step is usually carried out according to known identification strategies.

The following experimental examples are offered by way of illustration, and not by way of limitation.

### Further Experimental Section

### EXAMPLE I

### Preparation of Mab Minilibrary form the Coronary Plaque

The preparation of cDNA of Fab libraries from atherosclerotic plaques has been described in WO2009/037297.

Briefly: a sufficient amount of tissue (usually about 1-2 mg) was obtained from the atherosclerotic plaque from a number of patients with acute coronary syndrome and undergoing coronary atherectomy, and stored in liquid nitrogen, with the patient's informed consent.

The tissue was homogenized and the total mRNA was extracted according to conventional methodologies using a commercial kit for the extraction of mRNA.

Reverse transcription of mRNA was performed using a commercial kit for the retrotranscription of mRNA. The cDNA synthesis is performed according to standard procedures from the total mRNA primed with oligo(dT).

1 μl of cDNA was used for polymerase chain reaction. Reverse primers were designed in order to anneal to the segments of sequences coding for the constant region of heavy and light chains respectively. The PCR products of heavy and light chains of a Fab (variable region and the CH1 domain) amplified from the human biopsy, were cloned into a phagemidic vector (pRB32) to allow the combinatorial generation or heavy and light chain pairs exposed (phage display) onto the external phage surface the Fab fragment codified by the DNA cloned into the phagemid. This was obtained by cloning in frame the heavy chain fragment with a phage M13 membrane protein.

This allowed the generation of a combinatorial antibody Fab fragment phage-display libraries which have been screened with the lysates and antigens described in the following examples.

### EXAMPLE II

### Phage Display Libraries Selection by Biopanning on Cell, Carotid or Bacterial Lysates

Hep-2 (ATCC no CCL-23) cell lysates were prepared growing the cells in E-Mem (Invitrogen 0820234DJ) supplemented with antibiotic/antimycotic Solution (Invitrogen, Antibiotic/Antimycotic Solution, liquid 15240-062) and 10% FBS. Cells were regularly split 1:10 every five days. Five million cells were washed in PBS al lysed by using RIPA buffer (50 mM Tris HCl pH8+150 mM NaCl+1% NP-40+0.5% NA deoxycholate+0.1% SDS).

Carotid lysates were prepared from a portion (10 g) of human atherosclerotic carotid plaque obtained as described above. Carotid plaques were immersed in RIPA buffer and homogenized with Tissue ruptor (Qiagen).

Bacterial cell lysates were carried out inoculating a colony or a scratch in 10 ml of SB and growing bacteria at 37° C. overnight. Cultures of Staphylococcus aureus, Proteus mirabilis, Klebsiella pneumoniae, Enterococcus cloacae, Streptococcus pyogenes, Neisseriae gonhorreae, Listeria monocytogenes were grown. Overnight cultures were harvested by centrifugation (4500 RPM for 10 min.) and then resuspended in 1 ml of RIPA buffer and protease inhibitor PMSF (1 mM final). After three cycles of freeze-and-thawing (37° C. and −80° C.) and one cycle of sonication (90 sec at max power) the lysate was cleared by centrifugation (15000 RPM at 4° C. for 20 min) Moreover one colony of Haemophilus influenzae was seeded on blood agar plates and then grown at 37° C. overnight. Haemophilus inluenzae colonies were collected and resuspended in 500 μl of RIPA/PMSF. All lysates were stored at −20° C. until usage.

Bacterial lysates from Klebsiella pneumoniae were used to perform immunoaffinity selection by biopanning with the Fab libraries described in example 1 or as described for example in a Manual Phage Display. A laboratory manual, C. Barbas, D. Burton, J. Scott, G. Silvermann Jan. 2001 ed, CSH Press, Section I, Chapter 2. The night before biopanning, an ELISA plate was coated O/N at 4° C. with 50 μL/well of antigen(s) (100 ng/well) solution in appropriate coating buffer. After washing with deionised H2O, wells were blocked completely with PBS/BSA 3%, sealed and incubated in a humidified container for 1 h at 37° C. Fifty μL phage suspension was added to each well (total of about 1011 PFU) and incubated for at least 2 h at 37° C. after sealing the plate. Phage were removed from every well (and kept for titration), washed 10 times with PBS/Tween 0.05%. After intensive washing, phage bound to the antigen were eluted by washing each well with 50 μL of Elution Buffer (0.1M HCl, adjusted with glycine to pH=2.2, BSA 1 mg/ml) and adding to the eluent 3 μl of 2M Tris base. After the elution, 2 ml of fresh XL-1 blue E. coli (Stratagene) were infected with the eluted phage. After an incubation at 37° C. for 15 min, 10 ml of 37° C.-prewarmed Superbroth (SB) (20 μg/ml ampicillin and 10 μg/ml tetracycline) were added. After incubation for 1 hat 37° C. on a shaker, 100 ml of SB containing 100 μg/ml Amp and 10 μg/ml Tet were added to each 10 ml-culture and incubated for 1 h at 37° C. on a shaker A helper phage VCS M13 (total of 10¹² PFU) was used to infect the culture and incubated on the shaker for 2 h at 37° C. After addition of kanamycin (70 μg/ml) cultures were incubated on a shaker O/N at 30° C.

The day after, cells were spun down at 6000 RPM (Sorvall SS34), 4° C. for 15 min. and PEG8000 (Sigma-Aldrich) was added to the supernatant to a final volume of 20 ml. Phages were precipitated on ice for 30 min. and then centrifuged at 11,000 RPM (Sorvall SS34) for 20 min. at 4° C.) . Phages where then resuspend in 2 ml PBS/BSA 1%, and stored at 4° C. for subsequent biopanning rounds.

To verify the best coating conditions 1 μg of purified ompK36 was diluted in different buffers (PBS, Carbonate/Bicarbonate or NaHCO3) and the fraction coated on the plate was estimate by ELISA assay using anti-HIS-Peroxidase antibody.

The optimal conditions for coating were pH 5.0 with PBS buffer, overnight at 4° . Lysates and Omp were coated in PBS (pH 5.2)

After each elution and round of selection, eluted phage were titered in order to evaluate the efficacy of the enrichment procedure. The procedure was repeated four times allowing enrichment of selected populations.

### EXAMPLE III

### Sequencing

Clones obtained according to the biopanning procedure carried out as described in Example II and XIII for libraries derived from different coronary plaques, were sequenced for quantitative and qualitative analysis and sequencing by Big Dye chemistry and analyzed using ABI PRISM 3100 sequencer.

Screening of the minilibrary from a coronary plaque (IDNo 11: ID11LIB) provided for a heavy chain corresponding to SEQ ID NO: 1 (SEQINO:285) and coding for the amino acidic sequence corresponding to SEQ ID NO: 2 (SEQINO:286). The light chain correspond to SEQ ID NO: 3 (SEQINO:337) and coding for the amino acidic sequence corresponding to SEQ ID NO: 4 (SEQINO:338).

### EXAMPLE IV

### Expression System for SEQINO: 1 (SEQINO:285) and 3 (SEQINO:337)

A clone of the heavy chain (corresponding to SEQ ID NO:1 (SEQINO:285)) and a clone of the light chain (corresponding to SEQ ID NO:3 (SEQINO:337)) of the coronary plaque sample were selected for recombinant expression of the soluble Fab fragments according to the procedure described in Burioni R. et al. J. Immunol. Methods, 1998, 217(1-2):195-9.

Two expression systems were used: the first one allowing the production of flagged Fabs for purification (ref above) the second for secondary recognition (Burioni R. et al. Hum. Antibodies, 2001, 10(3-4):149-54).

Purified Fabs were tested in SDS-PAGE gel in non-reducing conditions showing a single band of approximately 50 kDa.

### EXAMPLE V

### Characterization of the Recombinant Fabs by Immunohistology

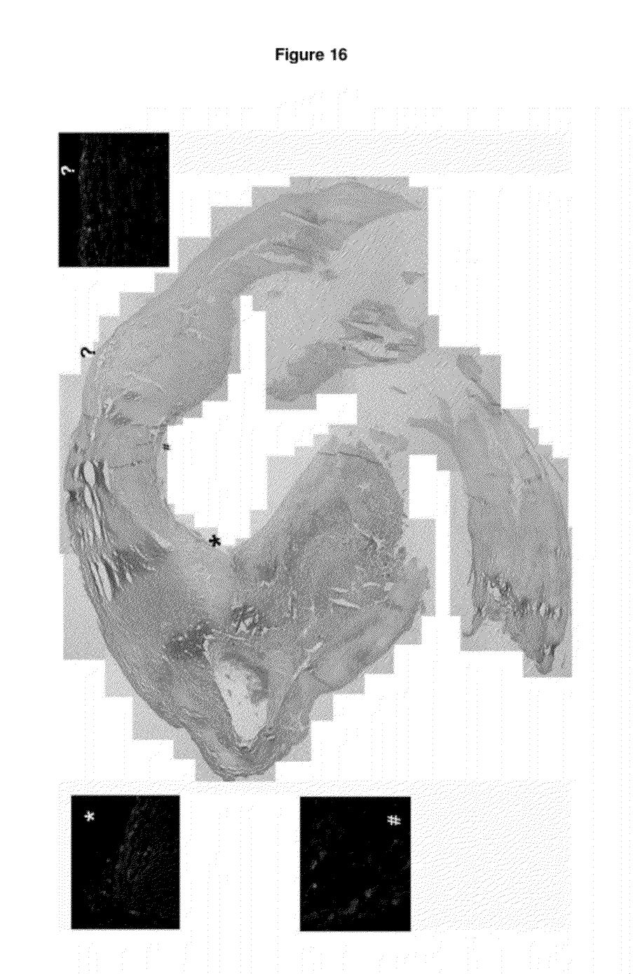

Carotid plaques harvested during TEA surgery, just after excision were fixed in 4% paraformaldehyde in PBS, cut in consecutive 2 mm thick rings, decalcified in EDTA solution when required, cryoprotected in 10% sucrose in PBS, embedded in OCT compound, then snap frozen in isopentane/liquid nitrogen and stored at −80° C. Cryosections (5 mm thick) were cut from every ring and stained with either Hematoxylin/Eosin or Movat's pentachrome, thus the morphology was examined in at least 4 sections/ring using an Eclipse 55i microscope equipped with DS-L1 camera and LUCIA 4.82 software (all from Nikon, Tokyo, J). Multiple images collected from each ring, were composed by LUCIA 4.82 software, to obtain a whole section picture. The presence of thin cap, foam cells, fibrolipids, ulcerations, necrotic/thrombotic core, calcifications, hemorrhage/thrombosis and inflammatory infiltrate were used to classify carotid plaques as stable, vulnerable, or unstable, following Virmani's modified AHA classification. Additional sections (10 μm thick) from rings with and without atheroma were selected and submitted for immunolabeling with anti CD138 antibody or with 7816Fab-FLAG detected with anti-FLAG clone M2 followed by rabbit-anti-Mouse IgG AlexaFluor488. DAPI stained the nuclei. Sections were examined using a Leica TCS SP2 confocal microscope (Leica Microsystems, Heidelberg, GmbH); 2D projection were obtained from Z-series of images and superposed by Adobe PhotoshopCS software. A histological section is shown in FIG. 16, together with the enlargments and immunofluorescence staining of three areas (identified by *, # or ?) of the carotid plaque (small panels: *, #, ?). The three panels stained by immunofluorescence show that a specific immonostaining, mainly cytoplasmic, is revealed after binding of the sections with the 7816Fab-FLAG. The nuclear fluorescence was due to DAPI staining.

### EXAMPLE VI

### Characterization of the Recombinant Fabs by Western Blotting on Carotid Lysates

Carotid plaques were macrodissected in 2 mm-thick rings and frozen in isopentane/liquid nitrogen. Every ring was considered as a single specimen. For each patient proteins were extracted from a stenotic and a non-stenotic ring by tissue homogenization in RIPA lysis buffer. The protein content of carotid plaque homogenates was quantified by Bradford assay. Western blot was performed on protein extracts (20 μg/lane) by using 10% Sodium Dodecyl Sulphate-PolyAcrylamide Gel Electrophoresis (SDS-PAGE). SDS-PAGE loading buffer was added to protein lysates before heating at 90° C. for 5 min. Gel was run for about 2 hours at 120V. PVDF membrane was treated according to the manufacturer's instruction and proteins were transferred to membrane at 0,350 mA for 2 hours. The membrane was stained by Ponceau red to verify the proper transfer. Then the membrane was blocked with a solution of PBS1×/BSA10% at 4° C. in agitation overnight. The day after the membrane was incubated with Fab E7816 (10 μg/ml) diluted in PBS1×/BSA5% for 1 hour at R.T. and then was washed three times with PBS1×/Tween-20 0.1%. Target (7816-FLAG) and reference proteins (bActin) were detected with specific antibodies (anti-FLAG clone M2 followed by Rabbit-anti Mouse IgG-HRP, and Rabbit-anti Mouse IgG-HRP, respectively—all from Sigma (cod: A9917, St. Louis, Mo.). The membrane was developed by SuperSignal West Pico Chemiluminescent Substrate (Pierce) according to the manufacture instructions. The mean pixel optical density (OD) for each band of the target protein, determined by densitometry, was normalized vs. β-Actin, to evaluate its quantity with respect to carotid plaque total cells. Stenotic and non stenotic rings from stable and unstable (determined by US) carotid plaque specimens were compared.

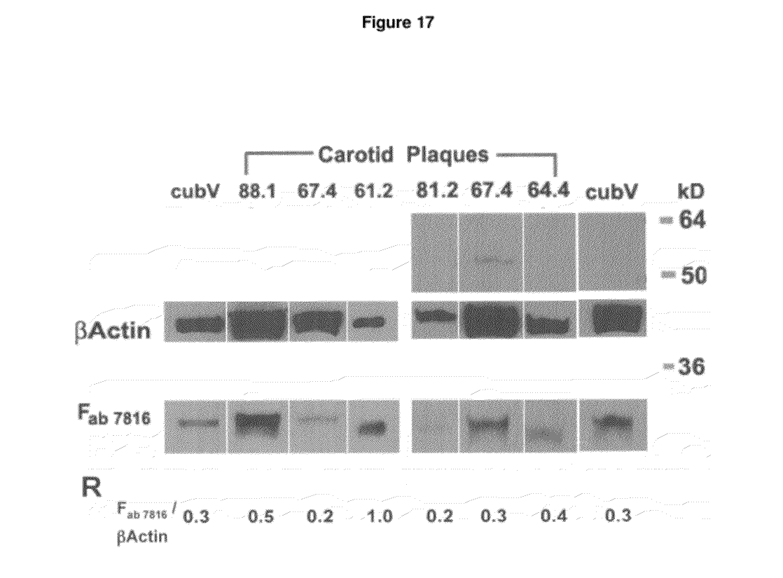

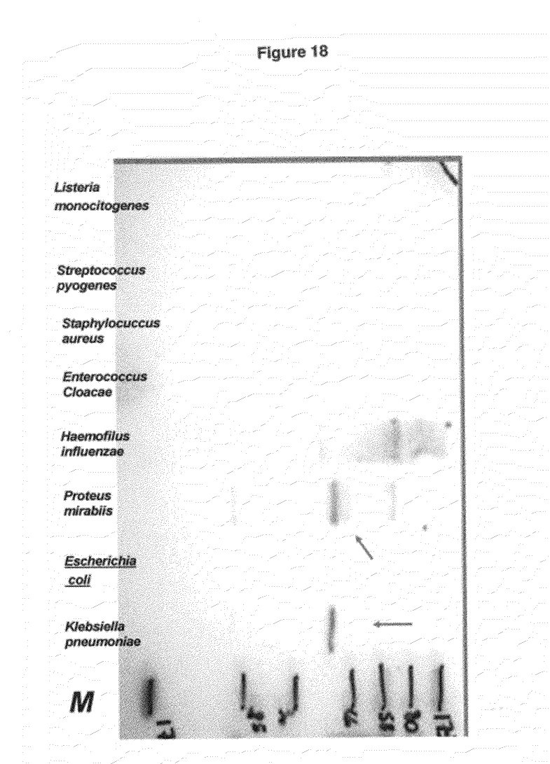

Sections used in WB (FIG. 17) were taken from distinct area of the lesion. From the WBs the antigen is differentially distributed among patients and seems to be differentially expressed in distinct regions of the plaque

### EXAMPLE VII

### Identification of a Bacterial Antigen with Fab7816

Lysates from Listeria monocitogenes, Streptococcus pyogenes, Staphylococcus aureus, Enterococcus cloacae, Haemophilus influenzae, Proteus mirabilis, Escherichia coli, Klebsiella pneumoniae, were prepared as described in Example II. SDS-PAGE was performed according to standard techniques. Briefly, protein concentration of all lysates was determined by BCA kit (Pierce) according to manufacturer instruction. SDS-PAGE loading buffer was added to protein lysates before heating at 90° C. for 5 min. 8 μg of total protein lysate were loaded in each well and the gel was run for about 2 hours at 120V. PVDF membrane was treated as described in manufacturer instruction and proteins were transferred to membrane at 0,350 mA for 2 hours. The membrane was stained by Ponceau red to verify the proper transfer. Then the membrane was blocked with a solution of PBS1×/BSA10% at 4° C. in agitation overnight. The day after the membrane was incubated with Fab E7816 (10 μg/ml) diluted in PBS1×/BSA5% for 1 hour at R.T. and then was washed three times with PBS1×/Tween-20 0,1%. The membrane was then incubated for 1 hour at R.T. with anti-Human Light Chain K antibody conjugated with HRP and then washed three times with PBS1×/Tween-20 0.1%. The membrane was developed by SuperSignal West Pico Chemiluminescent Substrate (Pierce) according to manufacture instructions and the films were impressed for 1 and 5 min.

The results shown in FIG. 18 indicates that in the conditions described for blotting, Fab7816 binds specifically to an antigen present in Klebsiella pneumoniae and in Proteus mirabilis (FIG. 18).

### EXAMPLE VIII

### ompK36 from Klebsiella Pneumoniae

The sequence of a putative bacterial antigen OmpK36 was amplified from Klebsiella lysate and cloned into a pET28b (Novagen) expression vector by using the following primer pairs containing two distinct 5′ restriction endonuclease sequences: OmpK36NcoIFW (SEQ ID NO:71) and OmpK36XhoIRW (SEQ ID NO:72); OmpK36BamHIFW (SEQ ID NO:73) and OmpK36XhoIRW (SEQ ID NO:72) and the following thermal profile was used: 95° C. 5 min then 95° C. 30min, 55° C. 30min and 72° C. 1 min. for 30 cycles; 72° C. 10 min. The amplification products and the pET28b vectors were double digested for 4 hours with the following restriction endonucleases: NcoI/XhoI (New England Biolabs) or BamHI/XhoI (New England Biolabs), loaded onto a 1% agarose gel and purified by using a Qiagen gel extraction kit. The corresponding digested amplicone was overnight ligated by using 10 units of T4 ligase (Roche). After transformation of bacterial cells (XL1-Blue E. coli) and plating, 8 individual colonies for each ligation were inoculated on LB broth and grown overnight. After plasmid purification (Qiagen Miniprep kit) the inserts were sequenced with the following primers: T7FWpET (SEQ ID NO:74) and T7termpET (SEQ ID NO:75). Correct cloning was confirmed and one plasmid for each ligation was used for ompK36 expression. OmpK36 nucleotide and amino acid sequences correspond to SEQ ID NO: 76 and 77 respectively.

The BamHI cloning allowed the generation of a 6×HISompK36 expression vector.

Expression were carried out using BL21 (DE3) E. coli bacterial cells transformed with selected plasmids, than the protein was purified by Ni-NTA purification (QiaExpressionist-Qiagen).

Western Blot was performed according to the procedure described in Example VII on total bacterial protein extracts obtained as described above from IPTG induced or non-induced BL21 (DE3) cultures or on purified ompK36 protein, loading 1 μg of purified protein or 20 μl for total lysate. The membrane was incubated for 1 hour at R.T. with anti-Human Light Chain K antibody conjugated with HRP and then washed three times with PBS1×/Tween-20 0,1%. The membrane was developed by SuperSignal West Pico Chemiluminescent Substrate (Pierce) according to manufacturer's instructions and the films were impressed for 1 and 5 min. WB was also developed with anti-HIS antibody (Roche) to verify the expression of HIS-tag at N-term of ompK36. Only the BamHI/XhoI cloned ompK36 has the HIS-tag as expected.

### EXAMPLE IX

### Expression and Purification of IgG7816 by Baculovirus in Insect Cells

Heavy and light chains of Fab 7816 were cloned in pAc-k-Fc vector (PROGEN cat No PR3001) to allow the production of baculovirus particle. Baculovirus amplification and subsequent expression of IgG7816 in Sf9 (Invitrogen) insect cells were performed following manufacturer instruction. The optimal MOI for IgG production and time for supernatant collection were evaluated. Sf9 cells were infected with a 0.2 MOI and grown for 5 days at 27° C. in agitation. At day 5, cell culture was centrifuged at 2000RPM for 15 min. and the supernatant was harvested, filtered and PMSF (Sigma-Aldrich) was added (final 1 mM). The supernatant was loaded on a chromatographic column with protein G resin (Flow Rate: 0.8 ml/min for 18 h). The resin was washed with 100-150 ml of PBS 1× pH:7.4 (F.R.: 1-2 ml/min), and the bound IgG was then eluted with 10 ml of Citric Acid 0.1 M pH: 5.2 and the solution neutralized at pH: 7 with 1.2 ml of Tris 2M pH:11. The optimization of IgG production with different MOIs (multiplicity of infection) and time for supernatant collection was performed using the following ELISA assay. In brief, the day before, an ELISA plate was coated with 100 ng/well of Anti-Human Fab Antibody [Sigma-Aldrich] in 25 μl at 4° C. O/N [or 2 h at 37° C]. The day after every well was washed with ddH2O and blocked with 180 μl/well of PBS/BSA 1% for lh at 37° C. The standard curve (from 250 ng/ml to 0.97 ng/ml, nine 1:2 serial dilutions of Standard IgGs(Sigma-Aldrich)) and at least three dilutions for every unknown samples (1:500, 1:1000 and 1:2000 at least in double) was added to the wells. In every well 40 μl of samples and standard were added and incubated for 1 h at 37° C. After 5 washes with PBS/Tween-20 0.1% an Anti-Human Fc antibody-HRP (SIGMA-Aldrich—diluted 1:8000), was added and incubated for 45-60 min at 37° C. After five washes with PBS/Tween-20 0.1% 40 μl of TMB solution (Pierce) in every well was added and incubated for 15 min. at 37° C. After addition of 40 μl of Stop Solution (H₂SO₄ 0.1M) in every well the plates were read at 450 nm

GraphPad Prism was used to draw the standard curve and interpolate the unknown sample quantities.

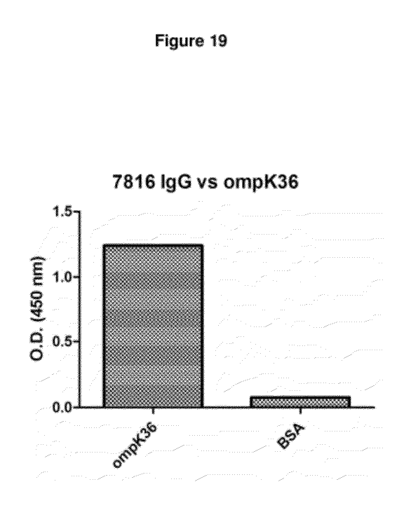

8 μg of OmpK36 was coated on a 96 well ELISA plate in PBS, pH 5.0, overnight at 4° C. The day after, after three washes with PBS, the plate was blocked with PBS/BSA 1%, and purified 7816IgGs were added (9 μg/ml) to each well. After washing three timed with PBS/tween-20 0.1%, an anti-Human Fc antibody-HRP (SIGMA-Aldrich—diluted 1:8000), was added and incubated for 45-60 min. at 37° C. After five washes with PBS/Tween-20 0.1% 40 μL of TMB solution (Pierce) in every well was added and incubated for 15 min. at 37° C. After addition of 40 μl of Stop Solution (H₂SO₄ 0.1 M) in every well the plates were read at 450 nm. In FIG. 19 is shown that 7816 when produced as a full IgG1 binds specifically to ompK36.

### EXAMPLE X

### 2D Electrophoresis Western Blotting (2DE-WB)

Sodium carbonate, glycine, sodium dodecyl sulfate (SDS) solution (20%) were purchased from Fluka. Ammonium persulfate, trizma base, urea, 3-[(3-Cholamidopropyl)dimethylammonio]-1-propanesulfonate (CHAPS), thiourea, bromophenol blue, iodoacetamide (IAA), N,N,N′,N′-tetramethylethylenediamine (TEMED), 1,4-dithioerythritol (DTE), PBS, Tween-20, bovine serum albumin (BSA), cocktail of protease inhibitors and antibodies (mouse anti-FLAG antibody; anti-mouse-peroxidase conjugated antibody) were obtained from Sigma. Agarose, glycerol (87% w/w), DryStrip Cover Fluid, DeStreak reagent, IPG (Immobilised pH Gradient) buffer pH 3-10NL, Immobiline DryStrip pH 3-10 NL, 7 cm, MW protein standards, PVDF membrane and ECL plus Chemiluminescent Substrate were from GE Healthcare. Coomassie brilliant blue G-250, 30% Acrylamide/Bis solution 37.5:1 and Bradford Protein Assay were purchased from Bio-Rad. Acetic acid and methanol were purchased from Merck. Zwittergent was from Calbiochem.

A piece of the coronary plaque was weighted (65 mg), cut in small fragments and directly smashed with a Dounce in 500 μl of the R5 buffer, containing: urea 8M, thiourea 2M, CHAPS 4%, Zwittergent 0.05%, 40 mM trizma base and a cocktail of protease inhibitors. The lysate was then sonicated for 5 minutes and centrifuged at 13,000 rpm for 30 minutes at 15° C. Supernatant protein content was evaluated by Bradford assay, using BSA dissolved in the same buffer, as standard. The proteins (200 μg), dissolved in R5 buffer (final volume 130 μl), were added to DeStreak (100 mM) and 2% IPG buffer pH 3-10NL, prior to loading the sample on strip pH 3-10NL, 7 cm. Total focusing run for the 1st dimension was 50,000 Vh. The 2nd dimension was carried out using 12.5% acrylamide SDS-PAGE. MW Standard proteins were run on the same gel. The gel was stained with colloidal Coomassie Brilliant Blue (cCBB).

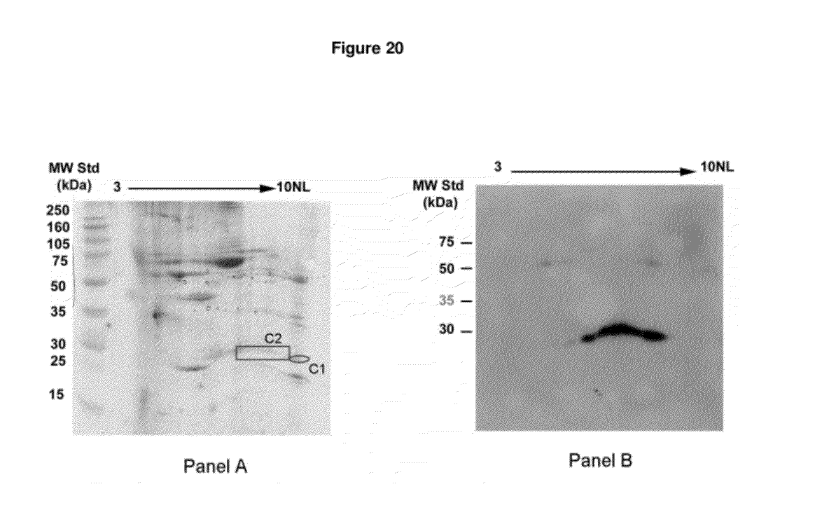

Similarly, 70 μg proteins dissolved in R5 buffer (final volume 130 μl), were added DeStreak (100 mM) and 2% IPG buffer pH 3-10NL, prior to loading the sample on strip pH 3-10NL, 7 cm for the gel to be blotted. Total focusing run for the 1st dimension was 50,000 Vh. The 2nd dimension was carried out using 12.5% acrylamide SDS-PAGE. MW Standard proteins were run on the same gel. Proteins were then transferred to PVDF membrane by semi-dry electroblotting and probed with primary antibody 7816FLAG. Immuno-complexes were visualized by incubation with anti-FLAG, peroxidase-conjugated secondary antibody and chemiluminescent detection (see FIG. 20, panel A and B).

### EXAMPLE XI

### Protein Identification by MALDI-ToF Mass Spectrometry

Protein corresponding to positive spots in WB were excised from the gel, destained, reduced with DTE (10 mM in ammonium bicarbonate 25 mM) at 56° C. for 30 minutes, alkylated with IAA (55 mM in ammonium bicarbonate 25 mM) in the dark at room temperature for 20 minutes. They were digested with trypsin and directly analysed by MALDI-ToF mass spectrometry, using alpha-cyano-4-hydroxycinnamic acid as matrix. We utilized Mascot software (Matrix Science, London) for protein searching in IPI_human_{—}20100623 database and the standard parameters (tryptic digest with a maximum of two missed cleavages, carboxyamidomethylation of cysteine residues, partial methionine oxidation and a mass tolerance of 50 ppm). We accepted an identification when the Mascot score was >66, with a good sequence coverage.

By comparison of images of 2DE and 2DE-WB described in Example X, it was decided to analyse the spots indicated in FIG. 20, panels A and B. They were recognized by the primary antibody we used. By mass spectrometry analysis transgelin 1 (SM22) was unequivocally identified as the putative natural (self) antigen recognized by Fab 7816 after sampling two distinct spots in the 2D gel. Data have been confirmed on purified protein by protein sequencing analysis.

### EXAMPLE XII

### Identification of Fab 5LcX

Biopanning of library 11 (ID11LIB) was carried out again on recombinant ompK36 coated on ELISA plates, for three rounds of selection.

Twenty clones were sequenced: the majority of clones resulted to have 7816 heavy and/or light sequences.

One clone, named Fab 5LcX, carried a different combination of heavy chain sequence SEQ ID NO 5 coding for the amino acidic sequence corresponding to SEQ ID NO: 6, and light chain sequence SEQ ID NO 7 coding for the amino acidic sequence corresponding to SEQ ID NO: 8.

### EXAMPLE XIII

### Identification of Fab 1630

The phage display Fab library from plaque 12 (ID12LIB) was screened once more by biopanning on the bacterial lysate and selected phages were tested on purified ompk36 in ELISA by biopanning carried out as described in Example II.

Seven selected clones were further sequenced, among them the most relevant was Fab1630 consisting of light chain SEQ ID NO 9 coding for the amino acidic sequence corresponding to SEQ ID NO: 10, and heavy chain SEQ ID NO 11 coding for the amino acidic sequence corresponding to SEQ ID NO: 12.

ELISA against purified OmpK36 carried out with a phage library made from peripheral blood sample of patient ID12 allowed to further identify the following heavy and light chains in the Sequence Listing: HC SEQ ID NO from 17 to 27 (odd numbers), coding for the aa SEQ ID NO from 18 to 28 (even numbers) and LC SEQ ID NO from 29 to 33 (odd numbers), coding for the aa SEQ ID NO from 30 to 34 (even numbers). Selected Fabs were found to comprise the following heavy and light chain pairs: Heavy Chain SEQ ID NO:18 and Light Chain selected from SEQ ID NO:30 and SEQ ID NO: 32; Heavy Chain SEQ ID NO 20 and Light Chain selected from SEQ ID NO: 32 and SEQ ID NO 34; Heavy Chain SEQ ID NO 22 and Light Chain SEQ ID NO: 32; Heavy Chain selected from SEQ ID NO 24 and SEQ ID NO 26 and Light Chain SEQ ID NO 30; Heavy Chain SEQ ID NO 28 and Light Chain selected among SEQ ID NO 12, SEQ ID NO 30 and SEQ ID NO 32.

### EXAMPLE XIV

### Western Blotting on Commercial Transgelin 1 with the Antibodies of the Invention

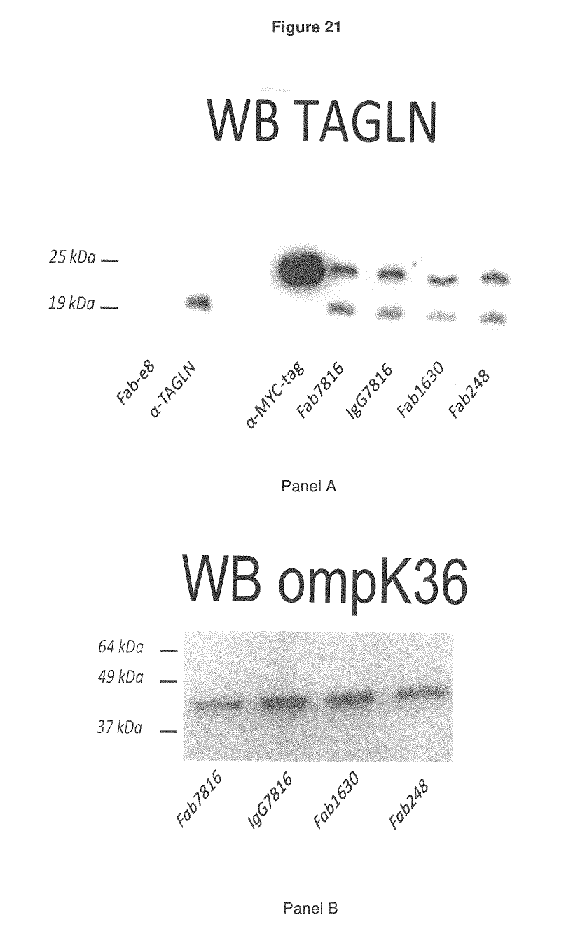

SDS-PAGE was performed according to standard techniques SDS-PAGE loading buffer was added to the recombinant transgelin (rTAGLN-Origene cod: TP302448) and was heated at 95° C. for 5 min then 4-500 ng of rTAGLN were loaded in each well and the gel was run for about 2 hours at 120V. PVDF membrane were treated as indicated in the manufacturer's instruction and proteins were transferred to membrane at 0.350 A for 2.5 hours. The membrane was stained by Ponceau red to verify the transfer. The membrane was then blocked with a solution of PBS1×/BSA10% at 4° C. in agitation overnight. The day after the membrane was incubated with purified Fabs (10 μg/ml) The membrane was incubated for 1 hour at r.t. with anti-Human Light Chain K antibody conjugated with HRP (Sigma cod: A7164) and then washed two times with PBS1×/Tween-20 0.1% and one time with PBS1×. Two wells were incubated with control antibody: anti-MYC-tag conjugated with HRP (Abcam cod: ab62928) and anti-TAGLN (Abnova cod: H00006876-M01). The anti-TAGLN was revealed by anti-Mouse-Fab conjugated with HRP (SIGMA cod: A9917). The membrane was developed by SuperSignal West Pico Chemiluminescent Substrate (Pierce) according to manufacture instructions and the films were impressed for 1 and 3 min A human unrelated monoclonal Fab against Influenza was used as a negative control and no signal was detected. The autoradiographic film is shown in FIG. 21, panel A. A positive band is visible also by using the IgG variant of Fab 7816.

### EXAMPLE XV

### Immunological Recognition of Purified OmpK36 by the Recombinant Antibodies of the Invention

The human recombinant Fabs 7816 1630 and 248 react with purified OmpK36 in western-blot.

400 ng of purified OmpK36His was loaded on polyacrylamide gel (all lanes) and SDS-PAGE was performed. Proteins were blotted on PVDF membrane for 2 h at 350 mA. After blocking with PBS/BSA10% the membrane was cut to single lanes and each lane was incubated for 1 h with Fabs at 10 μg/L. After three washes membrane was incubated with anti-Human Light Chain K antibody (Sigma). After three washes binding was revealed by commercial substrate used as manufacturer instructions (Pierce). Results are shown in FIG. 21 panel B.

Unrelated Fabs from the same library, used as negative controls, did not react against the same antigen.

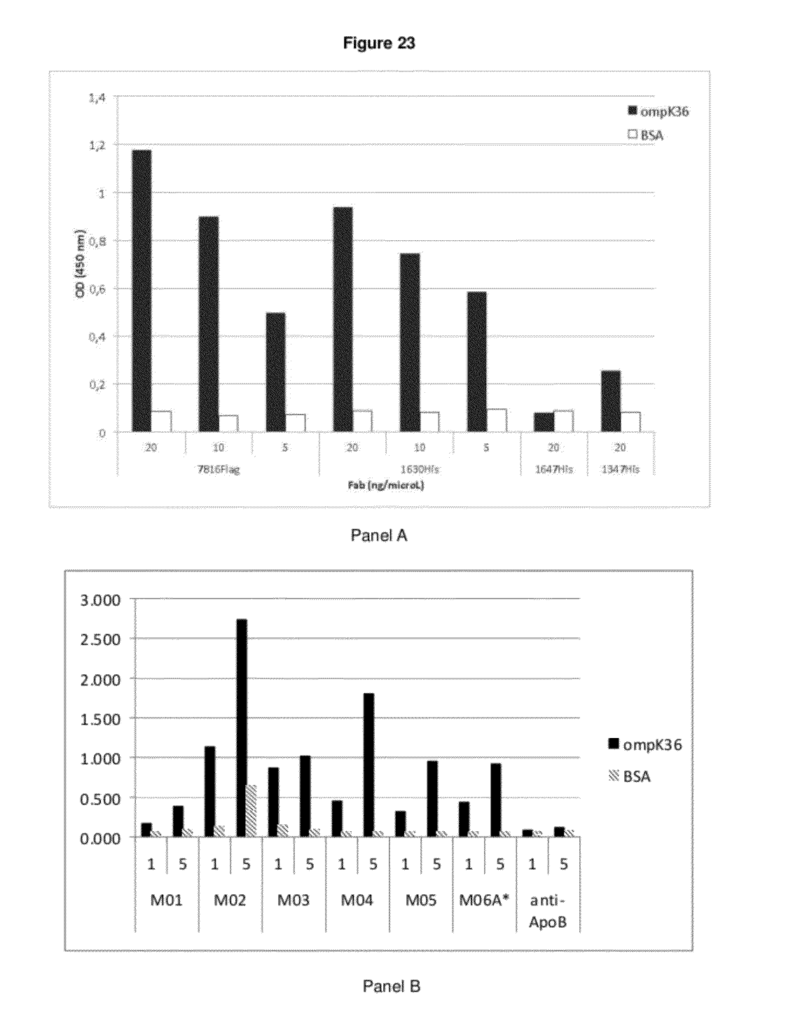

Two human recombinant Fabs (7816 and 1630), which have already been shown to recognize human transgelin in Western-blot, were also tested on OmpK36 by ELISA. Briefly, ELISA plates were coated with 4 μg/well of purified OmpK36; 7816 and 1630 Fabs were used at different concentration (20, 10 and 5 μg/mL) see FIG. 23, panel A. Binding of Fabs to OmpK36 was revealed by anti-kappa Light-Chain-HRP conjugated antibody (Sigma).

From the results of the ELISA assay, it can be concluded that the 2 antibodies characterized for their specific binding to TAGLN-1, show a concentration-dependent specific binding also to the bacterial protein OmpK36. Unrelated Fabs from the same library were used as negative controls at 20 μg/mL without showing any specific binding.

### EXAMPLE XVI

### Commercial Anti-Transgelin Monoclonal Antibodies Bind Purified OmpK36

The following commercial anti-TAGLN antibodies were tested for reactivity against OmpK36, by antigen coated ELISA. The antibody list, with their Abnova catalogue numbers, is as follows:
H00006876-M01 TAGLN monoclonal antibody (M01), clone 1E2
H00006876-M02 TAGLN monoclonal antibody (M02), clone 4B11
H00006876-M03 TAGLN monoclonal antibody (M03), clone 1C4
H00006876-M04 TAGLN monoclonal antibody (M04), clone 3A3
H00006876-M05 TAGLN monoclonal antibody (M05), clone 1D11
H00006876-M06A TAGLN monoclonal antibody (M06A), clone 3E6 (IgM)

This panel included 5 monoclonal mouse IgGs and one monoclonal mouse IgM. All monoclonals, except MO3, are obtained from mice immunized with the whole human TAGLN. M03 is obtained by immunisation of mice with partial recombinant TAGLN. Briefly, ELISA plates were coated with 4 μg of OmpK36 at 4° C. overnight. Commercial monoclonal antibodies against TAGLN were used following manufacturer's instructions (1 and 5 μg/mL) and revealed by anti-Mouse-Fab-HRP conjugated antibody (Sigma). A monoclonal mouse antibody against Human-ApoB100 (Meridian Life Science Inc.) was used as negative control at the same concentrations.

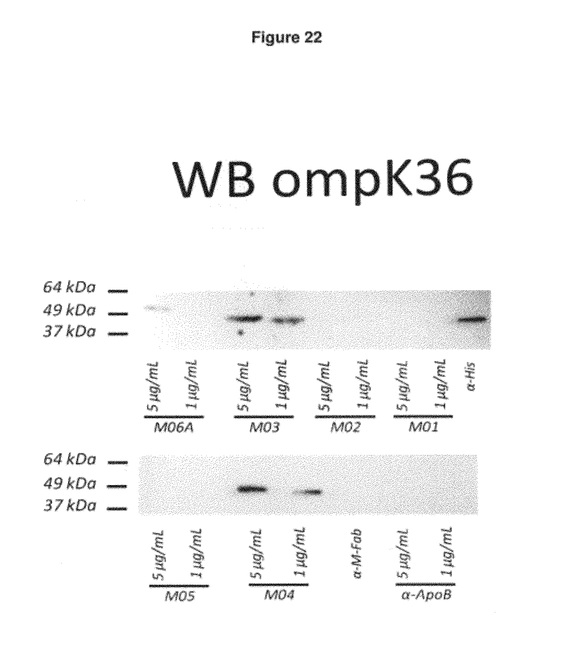

All commercial anti-TAGLN antibodies reacted, even if with different intensity, against OmpK36 in ELISA as compared to an unrelated antibody (α-ApoB): results are shown in FIG. 23, panel b). An immunoassay on OmpK36 was carried out with the same commercial anti-TAGLN antibodies by Western Blotting. 400 ng of purified OmpK36His was loaded on polyacrylamide gel (all lanes) and SDS-PAGE was performed. Proteins were blotted on PVDF membrane for 2 h at 350 mA. After blocking with PBS/Milk 10%, the membrane was cut to single lanes and each lane was incubated for 1 h with one of the commercial antibodies, at 1 or 5 μg/mL. After three washes the membranes were incubated with anti-Mouse-Fab HRP-conjugated antibody (Sigma). Binding was revealed by commercial substrate used as manufacturer instructions (Pierce) (FIG. 22, upper and lower panel). Anti-His-HRP was used as a positive control to reveal the correct transfer of OmpK36His. A non related mouse antibody (anti-Human-ApoB 100) was used as a negative control. In summary, when analysed in Western Blot using 10% milk as the blocking agent, M03, M04 and M06 were able to bind OmpK36. When 10% BSA was used as the blocking agent, only M06A was shown to bind OmpK36 in western blot.

### EXAMPLE XVII

### Sera from Patient with Acute Coronary Syndrome Recognize Purified OmpK36 and TAGLN

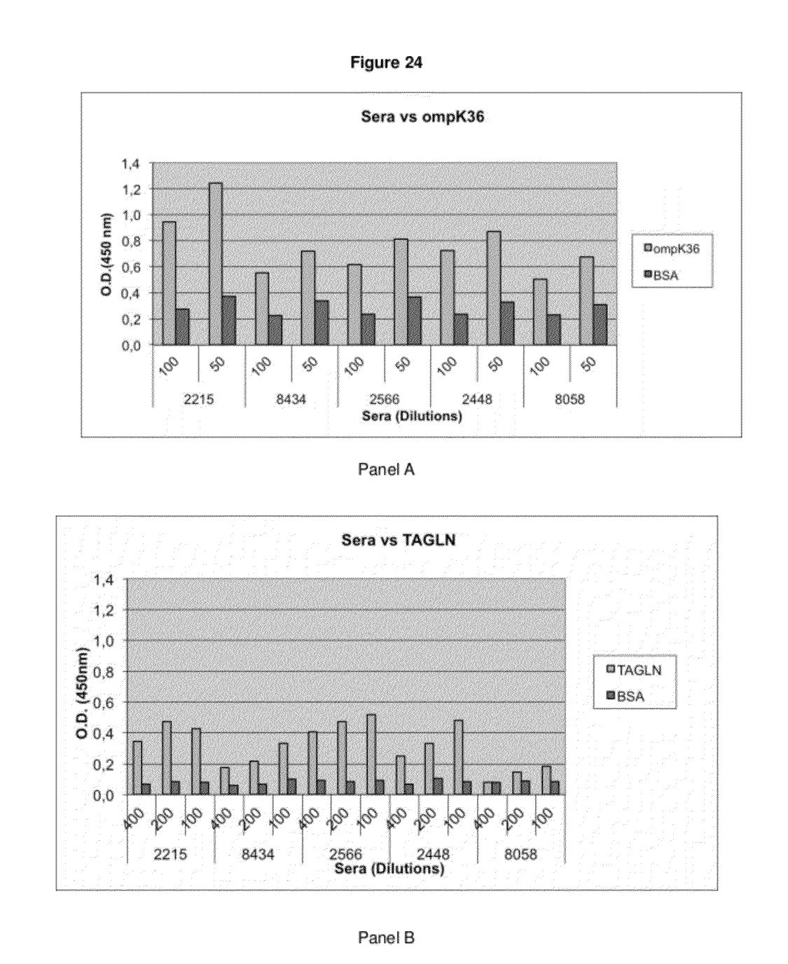

Five acute coronary syndrome patient sera were tested in ELISA for reactivity against purified OmpK36. Briefly, 300 ng of OmpK36 were coated in each well at 4° C. overnight. Then, the ELISA plate was blocked with PBS/BSA 1% for 1 h at 37° C. Sera were diluted 1:50 or 1:100 in PBS/BSA 1% and then incubated for 1 h at 37° C. After three washes with PBS/Tween 0.1%, a secondary antibody (anti-Human Fab HRP-conjugated) was incubated for 1 h at 37° C. After three washes with PBS/Tween 0.1%, the HRP signal was revealed by commercial substrate used as per manufacturer instructions (Pierce). Results are shown in FIG. 24, panel a). All tested sera showed a concentration dependent specific reactivity against OmpK36. An ELISA was carried out also against commercial TAGLN. The ELISA protocol was the same as for OmpK36. Sera were diluted 1:100, 1:200 or 1:400 in PBS/BSA 1%. As shown in FIG. 24, panel b), anti-TAGLN antibodies were detected in a dilution-dependent curve at least in three (8434, 2566 and 2448) out of five patients sera.

All references, including patents and published patent literature, cited or referred to herein are hereby incorporated by reference in their entirety.

We claim:

1. A human antibody which immunologically reacts with human transgelin or fragments thereof and with a protein with at least 50% similarity to OmpK36 (Outer membrane protein, Klebsiella, K36; GI: 295881594) or a fragment thereof.

2. The antibody according to claim 1 wherein said transgelin is transgelin 1.

3. The antibody according to claim 2 comprising a heavy and a light variable chain selected in the group consisting of:
a) a heavy chain variable region comprising SEQ ID NO: 2 (SEQ ID NO:286), and a light chain variable region comprising SEQ ID NO: 4 (SEQ ID NO:338);
b) a heavy chain variable region comprising SEQ ID NO: 6 (SEQ ID NO:408), and a light chain variable region comprising SEQ ID NO: 8;
c) a heavy chain variable region comprising SEQ ID NO: 10 (SEQ ID NO:428) and a light chain variable region comprising SEQ ID NO:12 (SEQ ID NO:438);
d) a heavy chain variable region comprising SEQ ID NO: 18 (SEQ ID NO:416) and a light chain variable region comprising SEQ ID NO:30 (SEQ ID NO:444);
e) a heavy chain variable region comprising SEQ ID NO: 18 (SEQ ID NO:416) and a light chain variable region comprising SEQ ID NO: 32 (SEQ ID NO:442);
f) a heavy chain variable region comprising SEQ ID NO: 20 and a light chain variable region comprising SEQ ID NO:32 (SEQ ID NO:442);
g) a heavy chain variable region comprising SEQ ID NO: 20 and a light chain variable region comprising SEQ ID NO: 34 (SEQ ID NO:450);
h) a heavy chain variable region comprising SEQ ID NO: 22 and a light chain variable region comprising SEQ ID NO: 32 (SEQ ID NO: 442);
i) a heavy chain variable region comprising SEQ ID NO: 24 and a light chain variable region comprising SEQ ID NO: 30 (SEQ ID NO: 444);
j) a heavy chain variable region comprising SEQ ID NO:26 (SEQ ID NO:398) and a light chain variable region comprising SEQ ID NO: 30 (SEQ ID NO: 444);
k) a heavy chain variable region comprising SEQ ID NO: 28 (SEQ ID NO:402) and a light chain variable region comprising SEQ ID NO: 12 (SEQ ID NO:438);
l) a heavy chain variable region comprising SEQ ID NO: 28 (SEQ ID NO:402) and a light chain variable region comprising SEQ ID NO: 30 (SEQ ID NO:444); and
m) a heavy chain variable region comprising SEQ ID NO: 28 (SEQ ID NO:402) and a light chain variable region comprising SEQ ID NO: 32 (SEQ ID NO:442).

4. The antibody according to claim 1 further recognizing an antigen in an atherosclerotic plaque sample.

5. The antibody according to any one of claims 1-4 for the preparation of an immunodiagnostic reagent for the detection of an atherogenic process, an atheromatous disease or ACS.

6. A method for detecting an antigen in an atherosclerotic plaque of an unknown sample comprising allowing said unknown biological sample to react with the antibody of claim 3 and detecting the antibody bound to the sample.

7. The method of claim 6 wherein detecting the antibody bound to the sample is indicative of the presence of an atheromatous disease, at risk of developing ACS, or the presence of an antigen associated with the atherosclerotic plaque

8. The method according to claim 7 wherein said atheromatous disease is selected from the group consisting of:
atherogenic ischemic or occlusive evolution in an arterial vessel;
Acute Coronary Syndrome comprising: unstable angina, ST Elevation Myocardial Infarction (STEMI), non STEMI myocardial infarction and related cardiovascular diseases;
intra-cerebral occlusive diseases;
peripheral artery occlusive diseases; and
non acute coronary diseases.

9. A method for the identification of an antigen associated with atherosclerotic plaque, its development or a diagnosis thereof, comprising contacting a sample from a patient with a monoclonal anti-transgelin 1 antibody further recognizing a protein with at least 50% similarity to OmpK36 (Outer membrane protein, Klebsiella, K36; GI: 295881594) or fragments thereof; and
detecting the antibody bound to the sample.

10. A method for detecting immunoglobulins against an antigen in an atherosclerotic plaque of an unknown biological sample comprising:
reacting said biological sample with human transgelin-1 or fragments thereof, optionally in competition with the antibody according to claim 1, and
detecting the immunoglobulin or antibody bound to the sample.

11. The method according to claim 10 further comprising allowing said unknown biological sample to react with a protein with at least 50% similarity to OmpK36 (Outer membrane protein, Klebsiella, K36; GI: 295881594) or a fragment thereof.

12. The method according to claim 10 wherein said method comprises a Western-blot or an ELISA.

13. The method according to claim 10 wherein detection of the immunoglobulin or antibody bound to the sample indicates an atherogenic process, an atheromatous disease or ACS.

14. A method for detecting an antigen in an atherosclerotic plaque of an unknown sample comprising allowing said unknown biological sample to react with an anti-transgelin 1 monoclonal antibody, said monoclonal antibody further characterized in that it cross reacts with a protein with at least 50% similarity to OmpK36 (Outer membrane protein, Klebsiella, K36; GI: 295881594) or a fragment thereof and detecting the antibody bound to the sample.

15. A method for detecting immunoglobulins against an antigen in an atherosclerotic plaque of an unknown biological sample comprising:
reacting said biological sample with human transgelin or a fragment thereof, optionally in competition with an anti-transgelin 1 monoclonal antibody wherein said anti-transgelin 1 monoclonal antibody further recognizes a protein with at least 50% similarity to OmpK36 (Outer membrane protein, Klebsiella, K36; GI: 295881594) or a fragment thereof, and
detecting the immunoglobulins bound to the sample.

16. The method according to claim 15 wherein said unknown biological sample or preparation thereof is further or in parallel reacted with a protein having at least 50% similarity to OmpK36 (Outer membrane protein, Klebsiella, K36; GI: 295881594) or fragments thereof.

17. The method according to claim 16 wherein the detection of the immunoglobulin bound to the sample is indicative of an atherogenic process, an atheromatous disease or ACS.

18. A method for selecting within a polypeptide library, a polypeptide with affinity for an antigen in an atherosclerotic plaque, comprising allowing said library, or a subset of it, to react with transgelin and a bacterial antigen with at least 50% similarity with OmpK36 (Outer membrane protein, Klebsiella, K36; GI: 295881594) or a fragment thereof.

19. The method of claim 18 wherein said transgelin is Transgelin 1 and said bacterial antigen is OmpK 36.
